(19) United States
(12) Patent Application Publication
(10) Pub. No.: US 2023/0117383 A1
(43) Pub. Date: Apr. 20, 2023

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Chi-Sik KIM, Gyeonggi-do (KR); Ji-Song JUN, Gyeonggi-do (KR); Soo-Yong LEE, Gyeonggi-do (KR); Seung-Hoon YOO, Gyeonggi-do (KR); Kyoung-Jin PARK, Gyeonggi-do (KR); Hyun-Woo KANG, Gyeonggi-do (KR); Hyun-Ju KANG, Gyeonggi-do (KR); So-Young JUNG, Gyeonggi-do (KR); Sang-Hee CHO, Gyeonggi-do (KR)

(21) Appl. No.: 17/734,347

(22) Filed: May 2, 2022

(30) Foreign Application Priority Data
May 26, 2021 (KR) 10-2021-0067841
Apr. 21, 2022 (KR) 10-2022-0049535

(51) Int. Cl. H01L 51/50; H01L 51/00

(52) U.S. Cl. CPC H01L 51/5024; H01L 51/0067; H01L 51/0073; H01L 51/0072; H01L 51/0054; H01L 51/0074; H01L 51/0071

(57) ABSTRACT

The present disclosure relates to a plurality of host materials, an organic electroluminescent compound, and an organic electroluminescent device comprising the same. By comprising the organic electroluminescent compound according to the present disclosure or by comprising a specific combination of compounds according to the present disclosure as a plurality of host materials, it is possible to produce an organic electroluminescent device having improved driving voltage, luminous efficiency, and/or lifetime properties compared to the conventional organic electroluminescent devices.

### TECHNICAL FIELD

The present disclosure relates to a plurality of host materials, an organic electroluminescent compound, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. However, in many applications such as TVs and lightings, the lifetime of OLEDs is insufficient and higher efficiency of OLEDs is still required. Typically, the higher the luminance of an OLED, the shorter the lifetime that the OLED has. Therefore, an OLED having high luminous efficiency and/or long lifetime characteristics is required for long time use and high resolution of a display.

In order to enhance luminous efficiency, driving voltage and/or lifetime, various materials or concepts for an organic layer of an OLED have been proposed. However, they were not satisfied in practical use.

Meanwhile, each of U.S. Pat. No. 9,397,307, Korean Patent Application Laying-Open No. 2018-0038834, and Korean Patent No. 1885898 discloses a plurality of host materials comprising a dibenzofuran or dibenzothiophene derivative and a biscarbazole derivative. However, the aforementioned references do not specifically disclose a specific combination of host materials claimed in the present disclosure, in particular a plurality of host materials comprising a second host material substituted with deuterium. In addition, it is continuously required to develop a light-emitting material having improved performances, for example, improved driving voltage, luminous efficiency, power efficiency and/or lifetime properties, as compared with the previously disclosed combination of specific compounds.

### DISCLOSURE OF INVENTION

### Technical Problem

The objective of the present disclosure is to provide a plurality of host materials capable of providing an organic electroluminescent device having improved driving voltage, luminous efficiency and/or lifetime properties. Another objective of the present disclosure is to provide an organic electroluminescent compound suitable for applying it to an organic electroluminescent device. Still another objective of the present disclosure is to provide an organic electroluminescent device having improved driving voltage, luminous efficiency and/or lifetime properties by comprising a specific compound, or by comprising a specific combination of compounds as host materials.

### Solution to Problem

As a result of intensive studies to solve the technical problems, the present inventors found that the above objective can be achieved by a plurality of host materials comprising a first host material(s) comprising the compound represented by the following formula 1, and a second host material(s) comprising the compound represented by the following formula 2. In addition, the present inventors found that the above objective can be achieved by an organic electroluminescent compound represented by the following formula 1-A or 1-B.

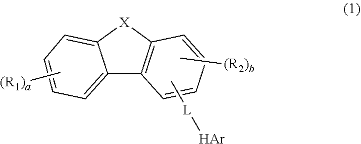

In formula 1,

X represents O, S or Se;

HAr represents a substituted or unsubstituted (3- to 30-membered)heteroaryl containing at least one nitrogen atom:

L represents a single bond, a substituted or unsubstituted (C6-C30)arylene, or a substituted or unsubstituted (3- to 30-membered)heteroarylene;

R₁ and R₂, each independently, represent hydrogen, deuterium, a substituted or unsubstituted (C6-C30)aryl, a substituted or unsubstituted (3- to 30-membered)heteroaryl, or —SiR₁₁R₁₂R₁₃;

R₁₁ to R₁₃, each independently, represent a substituted or unsubstituted (C1-C30)alkyl, a substituted or unsubstituted (C6-C30)aryl, or a substituted or unsubstituted (3- to 30-membered)heteroaryl; and

a represents an integer of 1 to 4, and b represents an integer of 1 to 3, in which if each of a and b is an integer of 2 or more, each of R₁ and each of R₂ may be the same or different.

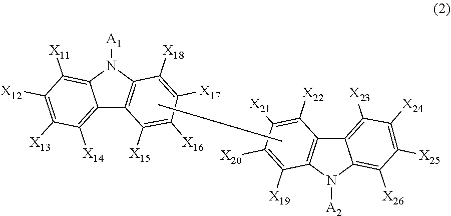

In formula 2,

A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;

X₁₁ to X₂₆, each independently, represent hydrogen, deuterium, a substituted or unsubstituted (C6-C30)aryl, or a substituted or unsubstituted (3- to 30-membered)heteroaryl; and

one of X₁₅ to X₁₈ and one of X₁₉ to X₂₂ are linked to each other to form a single bond, with the proviso that at least one of X₁₁, X₁₈, X₁₉ and X₂₆ is deuterium.

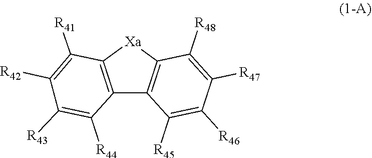

In formula 1-A,

X_{A} represents O or S; and

R₄₁ to R₄₈, each independently, represent hydrogen, deuterium, or a (C6-C18)aryl unsubstituted or substituted with at least one of deuterium, a (C1-C6)alkyl(s) and a (C6-C18)aryl(s), or are represented by the following formula 1-a, with the proviso that at least one of R₄₁ to R₄₈ is represented by the following formula 1-a:

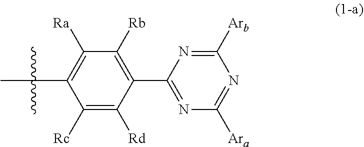

in formula 1-a,

Ra to Rd, each independently, represent hydrogen, deuterium, or a (C6-C18)aryl unsubstituted or substituted with at least one of deuterium, a (C1-C6)alkyl(s) and a (C6-C18)aryl(s); and

Arₐ and Ar_{b}, each independently, represent a phenyl unsubstituted or substituted with deuterium, a naphthyl unsubstituted or substituted with deuterium, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Arₐ and Ar_{b} comprises a substituted or unsubstituted carbazolyl.

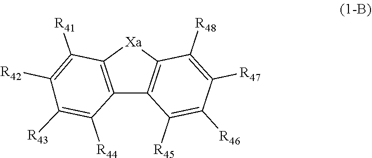

In formula 1-B,

Xₐ represent O or S;

R₄₁ to R₄₈, each independently, represent hydrogen, deuterium, or a (C6-C18)aryl unsubstituted or substituted with at least one of deuterium, a (C1-C6)alkyl(s) and a (C6-C18)aryl(s), or -L_{b}-HAr_{b}, with the proviso that at least one of R₄₁ to R₄₈ represents -L_{b}-HAr_{b};

L_{b} represents a naphthylene unsubstituted or substituted with deuterium; and

HAr_{b} is represented by the following formula 1-b:

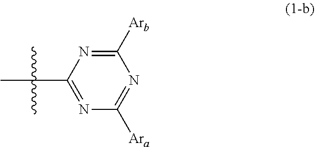

in formula 1-b,

Arₐ and Ar_{b}, each independently, represent a phenyl unsubstituted or substituted with deuterium, a naphthyl unsubstituted or substituted with deuterium, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Arₐ and Ar_{b} comprises a carbazolyl unsubstituted or substituted with at least one of deuterium and a (C6-C18)aryl(s), a dibenzofuranyl unsubstituted or substituted with deuterium, or a dibenzothiophenyl unsubstituted or substituted with deuterium.

### Advantageous Effects of Invention

The organic electroluminescent compound according to the present disclosure exhibits performances suitable for using it in an organic electroluminescent device. In addition, an organic electroluminescent device having low driving voltage, high luminous efficiency and/or excellent lifetime properties compared to conventional organic electroluminescent devices is provided by comprising a specific compound according to the present disclosure, or by comprising a specific combination of compounds according to the present disclosure as a plurality of host materials, and it is possible to produce a display system or a lighting system using the same.

### BRIEF DESCRIPTION OF THE FIGURES

FIG. 1 illustrates a graph showing the increase in bond dissociation energy related to deuteration.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, P, and Se, and preferably the group consisting of O, S, N, and Se. The above heterocycloalkyl may include tetrahydrofuran, pyrrolidine, thiolan, tetrahydropyran, etc. The term “(C6-C30)aryl” or “(C6-C30)arylene” is meant to be a monocyclic or fused ring radical derived from an aromatic hydrocarbon having 6 to 30 ring backbone carbon atoms. The above aryl or arylene may be partially saturated, and may comprise a spiro structure. The above aryl may include phenyl, biphenyl, terphenyl, naphthyl, binaphthyl, phenyinaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-8-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” or “(3- to 30-membered)heteroarylene” is meant to be an aryl or an arylene having 3 to 30 ring backbone atoms, and including at least one, preferably 1 to 4 heteroatoms selected from the group consisting of B, N, O, S, Si, P, and Se. The above heteroaryl or heteroarylene may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolephenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acrdinyl, 9-acrdinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted aryl, the substituted arylene, the substituted heteroaryl, the substituted heteroarylene, the substituted dibenzofuranyl, the substituted dibenzothiophenyl, the substituted carbazolyl, the substituted triazinyl, the substituted pyridyl, the substituted pyrimidinyl, the substituted quinazolinyl, the substituted benzoquinazolinyl, the substituted quinoxalinyl, the substituted benzoquinoxalinyl, the substituted quinolyl, the substituted benzoquinolyl, the substituted isoquinolyl, the substituted benzoisoquinolyl, the substituted triazolyl, the substituted pyrazolyl, the substituted naphthyridinyl, the substituted benzothienopyrimidinyl, the substituted pyridopyrazinyl, the substituted phenyl, the substituted biphenyl, the substituted terphenyl, the substituted naphthyl, the substituted fluorenyl, the substituted benzofluorenyl, the substituted triphenylenyl, the substituted fluoranthenyl, the substituted phenanthrenyl, the substituted dibenzofuranyl, the substituted carbazolyl, and the substituted dibenzothiophenyl, each independently, are at least one selected from the group consisting of deuterium; a halogen; a cyano; a carboxyl; a nitro; a hydroxyl; a (C1-C30)alkyl unsubstituted or substituted with deuterium; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium, a (C1-C30)alkyl(s), a (C6-C30)aryl(s), a (3- to 50-membered)heteroaryl(s), and a di(C6-C30)arylamino(s); a (C6-C30)aryl unsubstituted or substituted with at least one of deuterium, a cyano(s), a (C1-C30)alkyl(s), a (3- to 30-membered)heteroaryl(s), a mono- or di-(C6-C30)arylamino(s), and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (C1-C20)alkyl unsubstituted or substituted with at least one deuterium; a (C6-C25)aryl unsubstituted or substituted with at least one of deuterium, a (C6-C25)aryl(s), and a (5- to 25-membered)heteroaryl(s); and a (5- to 25-membered)heteroaryl unsubstituted or substituted with at least one of deuterium and a (C6-C25)aryl(s). According to another embodiment of the present disclosure, the substituent(s), each independently, are at least one selected from the group consisting of deuterium; a (C1-C20)alkyl unsubstituted or substituted with at least one deuterium; a (C6-C18)aryl unsubstituted or substituted with at least one of deuterium, a (C6-C18)aryl(s), and a (5- to 20-membered)heteroaryl(s); and a (5- to 20-membered)heteroaryl unsubstituted or substituted with at least one of deuterium and a (C6-C18)aryl(s). For example, the substituent(s) may be one selected from the group consisting of deuterium; a methyl; a phenyl unsubstituted or substituted with at least one of deuterium, a dibenzofuranyl(s), and a carbazolyl(s); a naphthyl unsubstituted or substituted with at least one deuterium; a naphthylphenyl; a phenylnaphthyl; a biphenyl unsubstituted or substituted with at least one deuterium; a terphenyl unsubstituted or substituted with at least one deuterium; a triphenylenyl unsubstituted or substituted with at least one deuterium; a fluorenyl unsubstituted or substituted with at least one of deuterium and a phenyl(s); a spirobifluorenyl unsubstituted or substituted with at least one deuterium; a dibenzofuranyl unsubstituted or substituted with at least one of deuterium and a phenyl(s); a dibenzothiophenyl unsubstituted or substituted with at least one of deuterium and a phenyl(s); a carbazolyl unsubstituted or substituted with at least one of deuterium and a phenyl(s); and a benzocarbazolyl.

In the present disclosure, heteroaryl, heteroarylene, and heterocycloalkyl may, each independently, contain at least one heteroatom selected from the group consisting of B, N, O, S, Si, R and Se. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

A plurality of host materials of the present disclosure comprise a first host material and a second host material, in which the first host material comprises at least one compound represented by formula 1, and the second host material comprises at least one compound represented by formula 2.

In formula 1, X represents O, S or Se.

In formula 1, HAr represents a substituted or unsubstituted (3- to 30-membered)heteroaryl containing a nitrogen atom(s). HAr contains preferably at least two nitrogen atoms, and more preferably at least three nitrogen atoms. According to one embodiment, HAr represents a (5- to 25-membered)heteroaryl containing a nitrogen atom(s) and unsubstituted or substituted with at least one of a (C6-C30)aryl(s) and a (3- to 30-membered)heteroaryl(s). According to another embodiment, HAr represents a (5- to 20-membered)heteroaryl containing a nitrogen atom(s) and unsubstituted or substituted with two (C6-C30)aryls, or a (C6-C30)aryl and a (3- to 30-membered)heteroaryl. Specifically, HAr may be a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzothienopyrimidinyl, a substituted or unsubstituted carbazolyl, or a substituted or unsubstituted pyridopyrazinyl. The substituent(s) of the substituted triazinyl, the substituted pyridyl, the substituted pyrimidinyl, the substituted quinazolinyl, the substituted benzoquinazolinyl, the substituted quinoxalinyl, the substituted benzoquinoxalinyl, the substituted quinolyl, the substituted benzoquinolyl, the substituted isoquinolyl, the substituted benzoisoquinolyl, the substituted triazolyl, the substituted pyrazolyl, the substituted naphthyridinyl, the substituted benzothienopyrimidinyl, the substituted carbazolyl, and the substituted pyridopyrazinyl, each independently, may be a (C6-C30)aryl unsubstituted or substituted with at least one of deuterium, a tri(C6-C30)arylsilyl(s), and a (3- to 30-membered)heteroaryl(s); or a (3- to 30-membered)aryl unsubstituted or substituted with a (C6-C30)aryl(s). For example, HAr may be a substituted triazinyl or a substituted pyrimidinyl, in which the substituent(s) of the substituted triazinyl and the substituted pyrimidinyl, each independently, may be at least one, preferably any two selected from the group consisting of a phenyl unsubstituted or substituted with at least one of deuterium, a dibenzofuranyl(s), a carbazolyl(s), and a triphenylsilyl(s); a naphthyl; a naphthylphenyl; a phenylnaphthyl; a biphenyl unsubstituted or substituted with at least one deuterium; a terphenyl; a triphenylenyl; a diphenylfluorenyl; a spirobifluorenyl; a dibenzofuranyl unsubstituted or substituted with at least one deuterium and a phenyl(s); a dibenzothiophenyl unsubstituted or substituted with at least one deuterium; a carbazolyl unsubstituted or substituted with at least one of deuterium and a phenyl(s); and a benzocarbazolyl, which may be further substituted with deuterium.

In formula 1, L represents a single bond, a substituted or unsubstituted (C6-C30)arylene, or a substituted or unsubstituted (3- to 30-membered)heteroarylene. According to one embodiment, L represents a single bond, or a substituted or unsubstituted (C6-C25)arylene. According to another embodiment, L represents a single bond, or a (C6-C18)arylene unsubstituted or substituted with deuterium. For example, L may be a single bond, a phenylene unsubstituted or substituted with deuterium, or a naphthylene unsubstituted or substituted with deuterium.

In formula 1, R₁ and R₂, each independently, represent hydrogen, deuterium, a substituted or unsubstituted (C6-C30)aryl, a substituted or unsubstituted (3- to 30-membered)heteroaryl, or —SiR₁₁R₁₂R₁₃. According to one embodiment, R₁ and R₂, each independently, represent hydrogen, deuterium, a substituted or unsubstituted (C6-C25)aryl, or a substituted or unsubstituted (5- to 25-membered)heteroaryl. According to another embodiment, R₁ and R₂, each independently, represent hydrogen, deuterium, a (C6-C25)aryl unsubstituted or substituted with a (C6-C18)aryl(s), or a (5- to 25-membered)heteroaryl unsubstituted or substituted with a (C6-C18)aryl(s). For example, R₁ and R₂, each independently, may be hydrogen, deuterium, a phenyl, a biphenyl, a fluorenyl substituted with a phenyl(s), a triphenylenyl, a spirobifluorenyl, a dibenzothiophenyl, a dibenzofuranyl, a dibenzoselenophenyl, a carbazolyl substituted with a phenyl(s), or a benzothienocarbazolyl, etc.

R₁₁ to R₁₃, each independently, represent a substituted or unsubstituted (C1-C30)alkyl, a substituted or unsubstituted (C6-C30)aryl, or a substituted or unsubstituted (3- to 30-membered)heteroaryl.

In formula 1, a represents an integer of 1 to 4, and b represents an integer of 1 to 3, in which if each of a and b is an integer of 2 or more, each of R₁ and each of R₂ may be the same or different.

The formula 1 may be represented by the following formula 1-1.

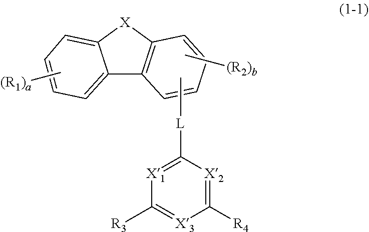

In formula 1-1, X, R₁, R₂, L, a, and b are as defined in formula 1.

In formula 1-1, X′₁ to X′₃, each independently, represent CR′ or N, in which R′ represents hydrogen or deuterium, and at least two of X₁ to X′₃ represent N. Specifically, any two of X′₁ to X′₃ may be N and the other may be CR′; or all of X′₁ to X′₃ may be N.

In formula 1-1, R₃ and R₄, each independently, represent a substituted or unsubstituted (C6-C30)aryl, or a substituted or unsubstituted (3- to 30-membered)heteroaryl. R₃ and R₄ are the same as or different from each other. According to one embodiment, R₃ and R₄, each independently, represent a substituted or unsubstituted (C6-C25)aryl, or a substituted or unsubstituted (5- to 25-membered)heteroaryl. According to another embodiment, Ra and R₄, each independently, represent a (C6-C25)aryl unsubstituted or substituted with at least one of deuterium, a tri(C6-C30)arylsilyl(s) and a (5- to 25-membered)heteroaryl(s); or a (5- to 25-membered)heteroaryl unsubstituted or substituted with a (C6-C18)aryl(s). For example, R₃ and R₄, each independently, may be a phenyl unsubstituted or substituted with at least one of deuterium, a dibenzofuranyl(s), a carbazolyl(s), and a triphenylsilyl(s); a biphenyl unsubstituted or substituted with at least one deuterium; a naphthyl; a naphthylphenyl; a phenylnaphthyl; a terphenyl; a triphenylenyl; a diphenylfluorenyl; a spirobifluorenyl; a dibenzofuranyl unsubstituted or substituted with a phenyl(s); a dibenzothiophenyl unsubstituted or substituted with a phenyl(s); a carbazolyl unsubstituted or substituted with a phenyl(s); or a benzocarbazolyl, etc., which may be further substituted with deuterium.

The formula 1-1 may be represented by any one of the following formulas 1-1-1 to 1-1-4.

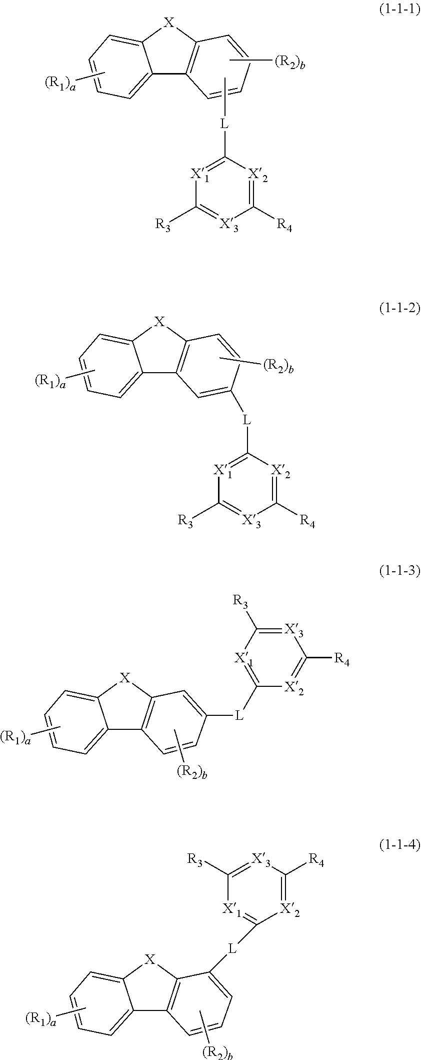

In formulas 1-1-1 to 1-1-4, X, X′₁ to X′₃, R₁ to R₄, L, a, and b are as defined in formula 1-1.

In formula 2, A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment, A₁ and A₂, each independently, represent a substituted or unsubstituted (C6-C25)aryl, a substituted or unsubstituted dibenzofuranyl, a substituted or unsubstituted dibenzothiophenyl, or a substituted or unsubstituted carbazolyl. Specifically, A₁ and A₂, each independently, may be a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl. The substituent(s) of the substituted aryl, the substituted phenyl, the substituted biphenyl, the substituted terphenyl, the substituted naphthyl, the substituted fluorenyl, the substituted benzofluorenyl, the substituted triphenylenyl, the substituted fluoranthenyl, the substituted phenanthrenyl, the substituted dibenzofuranyl, the substituted carbazolyl, and the substituted dibenzothiophenyl, each independently, may be at least one of deuterium, a (C6-C30)aryl(s), and a (3- to 30-membered)heteroaryl(s), preferably at least one of deuterium, a (C6-C18)aryl(s), and a (5- to 20-membered)heteroaryl(s). For example, A₁ and A₂, each independently, may be a phenyl unsubstituted or substituted with at least one of deuterium, a naphthyl(s), a triphenylenyl(s), a dibenzofuranyl(s), and a dibenzothiophenyl(s); a naphthyl unsubstituted or substituted with at least one of deuterium and a phenyl(s); a biphenyl unsubstituted or substituted with at least one deuterium; a terphenyl unsubstituted or substituted with at least one deuterium; a triphenylenyl unsubstituted or substituted with at least one deuterium; a dibenzofuranyl unsubstituted or substituted with at least one of deuterium and a phenyl(s); a dibenzothiophenyl unsubstituted or substituted with at least one of deuterium and a phenyl(s); or a carbazolyl unsubstituted or substituted with at least one of deuterium, a phenyl(s), and a naphthyl(s).

In formula 2, X₁₁ to X₂₆, each independently, represent hydrogen, deuterium, a substituted or unsubstituted (C6-C30)aryl, or a substituted or unsubstituted (3- to 30-membered)heteroaryl. Any one of X₁₅ to X₁₈ and any one of X₁₉ to X₂₂ are linked to each other to form a single bond. At least one, preferably two, more preferably three, and even more preferably four, of X₁₁, X₁₈, X₁₉ and X₂₆ are deuterium.

According to one embodiment, in the compound represented by formula 2, the deuterium substitution rate is 90% or more, preferably 94% or more, and more preferably 97% or more, of the total number of hydrogen. When the compound represented by formula 2 is substituted in the aforementioned deuterium substitution rate, the bond dissociation energy related to deuteration may increase to enhance the stability of a compound. An organic electroluminescent device comprising said compound may exhibit an improved lifetime property.

The formula 2 may be represented by any one of the following formulas 2-1 to 2-8.

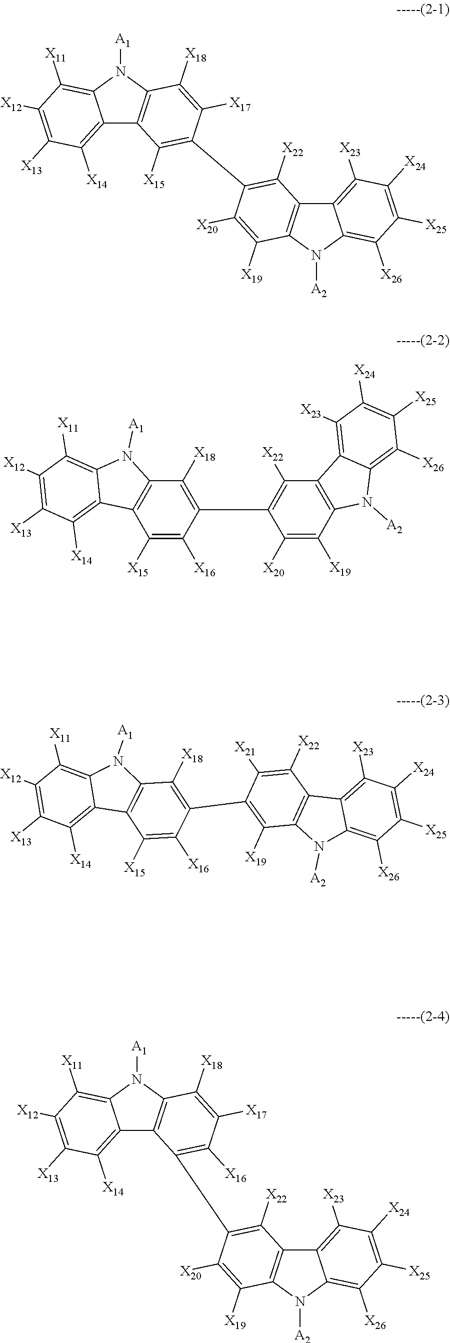

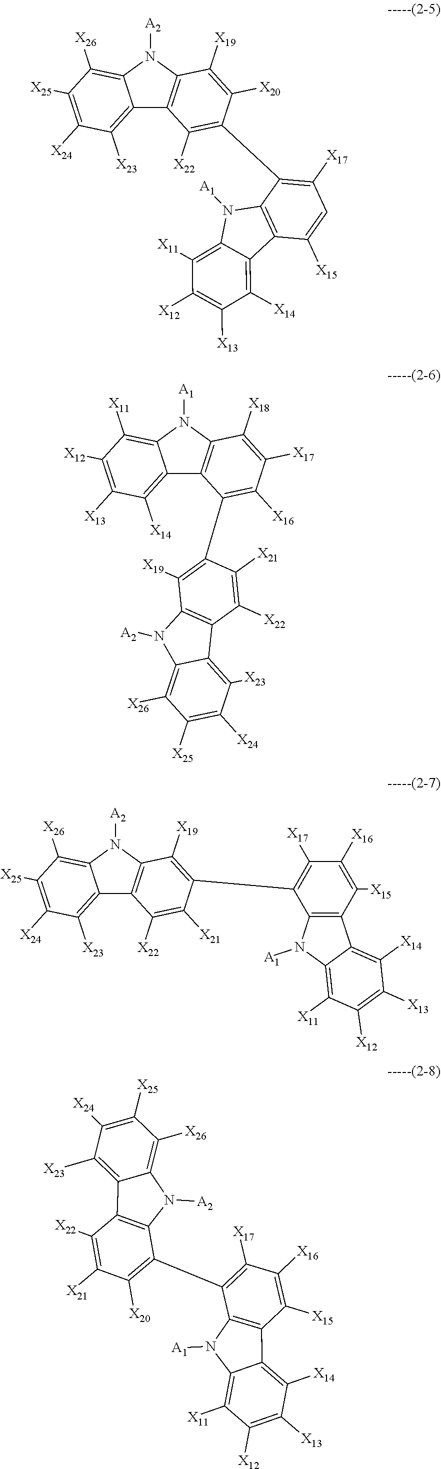

In formulas 2-1 to 2-8, A₁, A₂ and X₁₁ to X₂₆ are as defined in formula 2.

The compound represented by formula 1 may be at least one selected from the group consisting of the following compounds, but is not limited thereto.

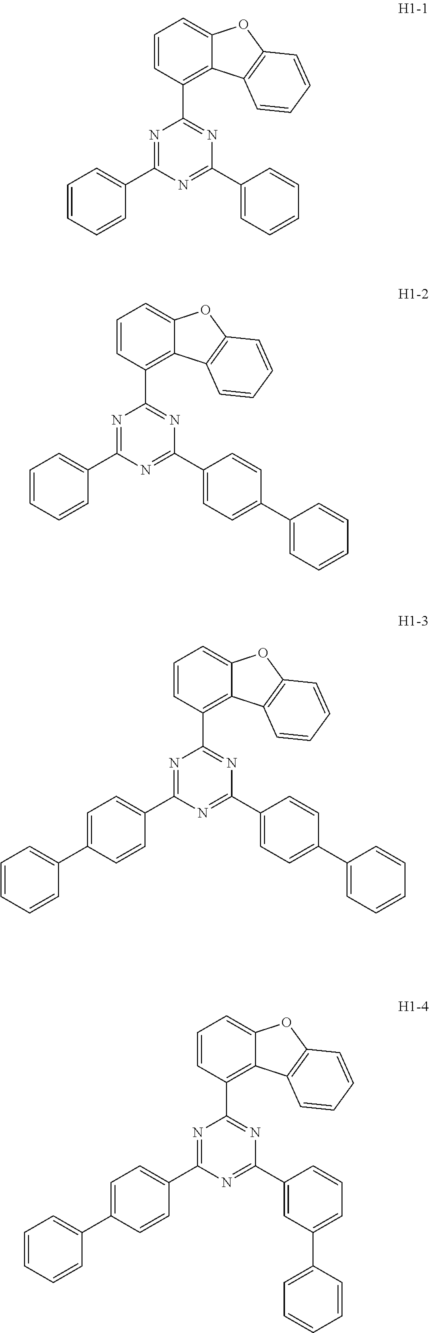 [SMILES: c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1]

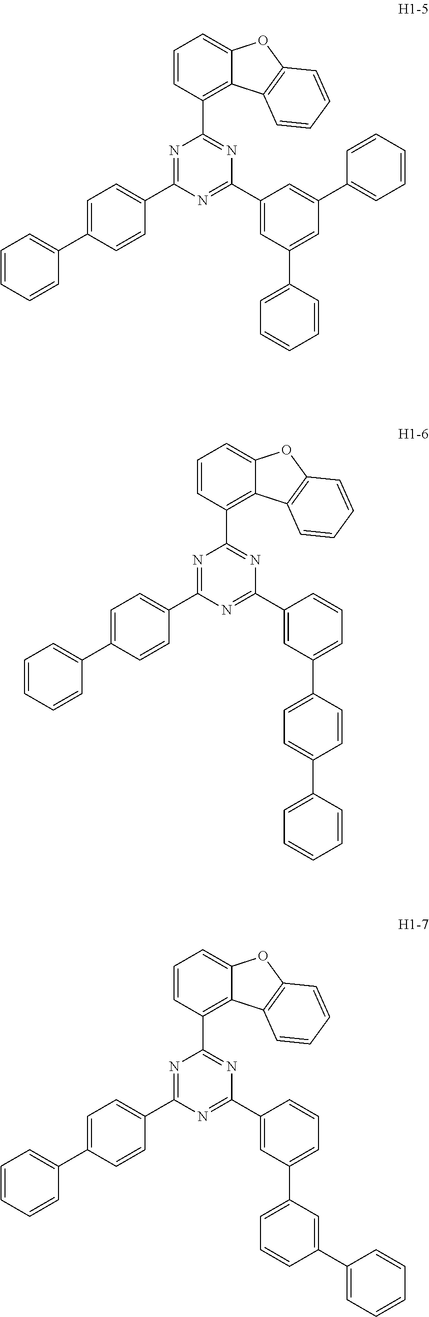 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1]

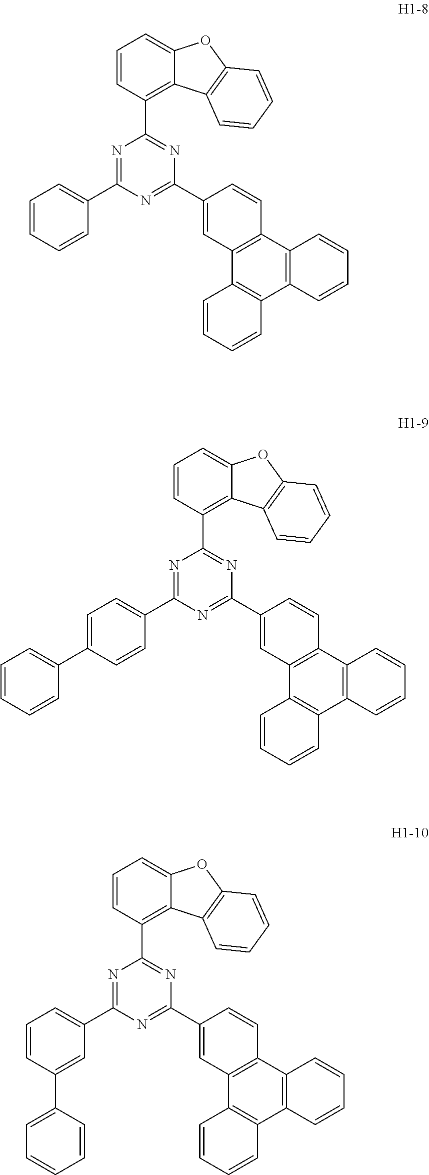 [SMILES: c1ccc(-c2nc(-c3ccc4c5ccccc5c5ccccc5c4c3)nc(-c3cccc4oc5ccccc5c34)n2)cc1 ; c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1]

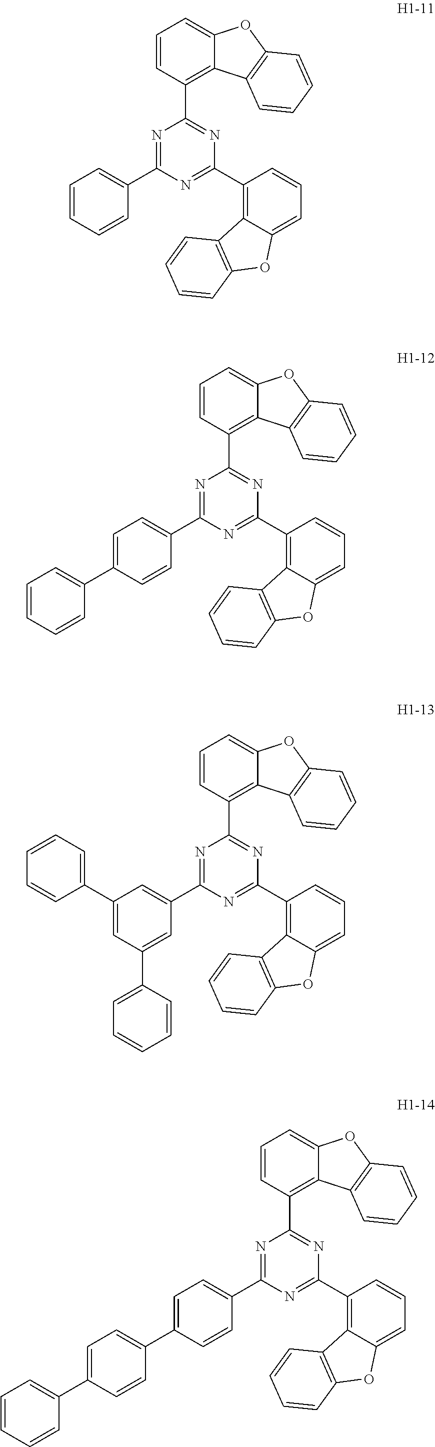 [SMILES: c1ccc(-c2nc(-c3cccc4oc5ccccc5c34)nc(-c3cccc4oc5ccccc5c34)n2)cc1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1 ; c1ccc(-c2ccc(-c3ccc(-c4nc(-c5cccc6oc7ccccc7c56)nc(-c5cccc6oc7ccccc7c56)n4)cc3)cc2)cc1]

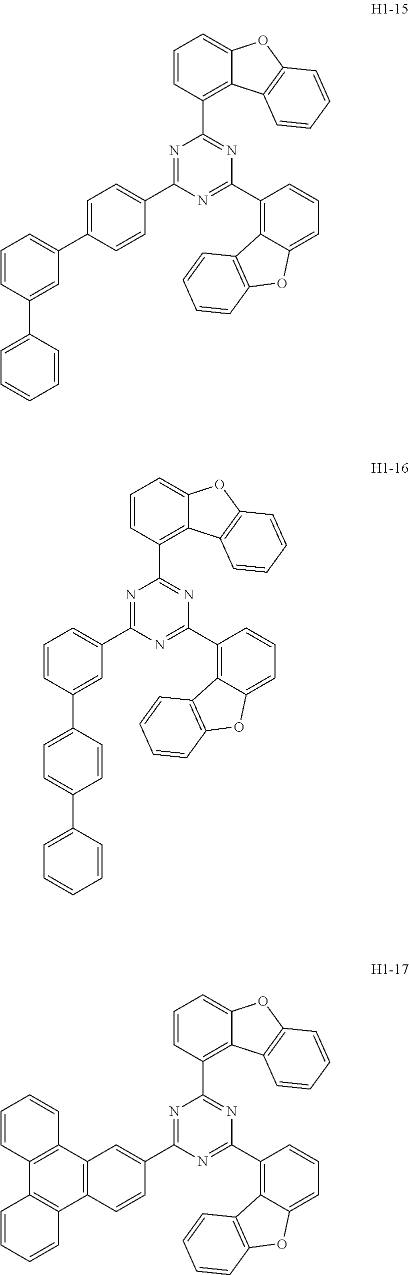 [SMILES: c1ccc(-c2cccc(-c3ccc(-c4nc(-c5cccc6oc7ccccc7c56)nc(-c5cccc6oc7ccccc7c56)n4)cc3)c2)cc1 ; c1ccc(-c2ccc(-c3cccc(-c4nc(-c5cccc6oc7ccccc7c56)nc(-c5cccc6oc7ccccc7c56)n4)c3)cc2)cc1 ; c1ccc2c(c1)oc1cccc(-c3nc(-c4ccc5c6ccccc6c6ccccc6c5c4)nc(-c4cccc5oc6ccccc6c45)n3)c12]

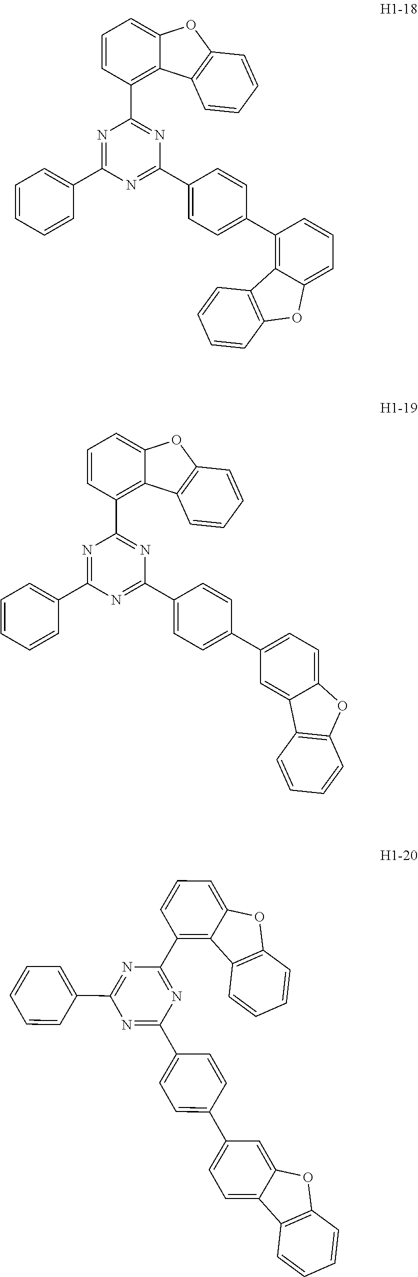 [SMILES: c1ccc(-c2nc(-c3ccc(-c4cccc5oc6ccccc6c45)cc3)nc(-c3cccc4oc5ccccc5c34)n2)cc1 ; c1ccc(-c2nc(-c3ccc(-c4ccc5oc6ccccc6c5c4)cc3)nc(-c3cccc4oc5ccccc5c34)n2)cc1 ; c1ccc(-c2nc(-c3ccc(-c4ccc5c(c4)oc4ccccc45)cc3)nc(-c3cccc4oc5ccccc5c34)n2)cc1]

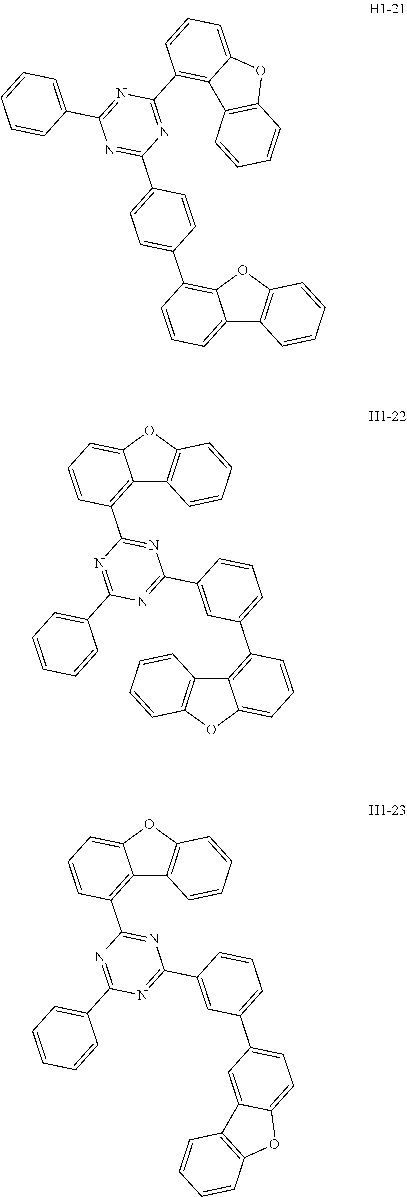 [SMILES: c1ccc(-c2nc(-c3ccc(-c4cccc5c4oc4ccccc45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3cccc(-c4ccc5oc6ccccc6c5c4)c3)nc(-c3cccc4oc5ccccc5c34)n2)cc1]

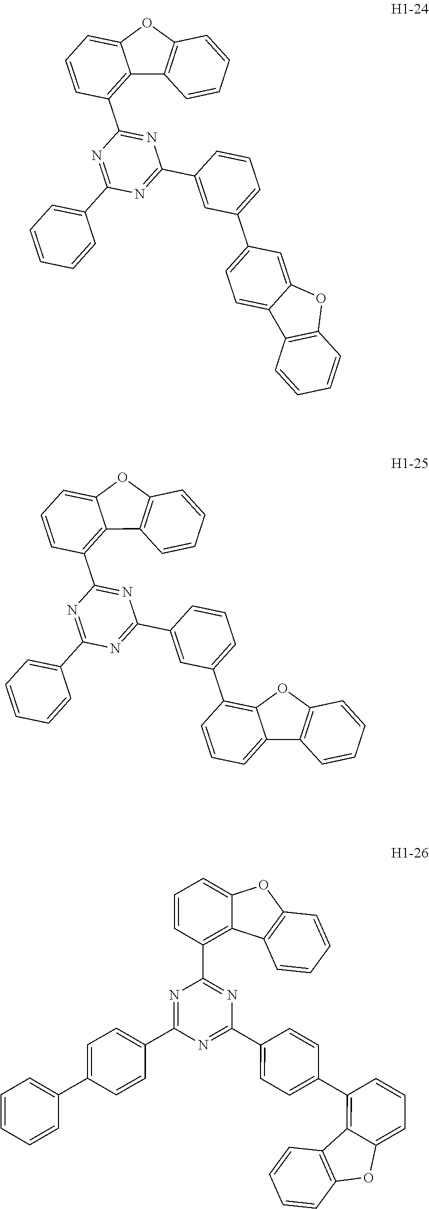 [SMILES: c1ccc(-c2nc(-c3cccc(-c4ccc5c(c4)oc4ccccc45)c3)nc(-c3cccc4oc5ccccc5c34)n2)cc1 ; c1ccc(-c2nc(-c3cccc(-c4cccc5c4oc4ccccc45)c3)nc(-c3cccc4oc5ccccc5c34)n2)cc1 ; c1ccc(-c2ccc(-c3nc(-c4ccc(-c5cccc6oc7ccccc7c56)cc4)nc(-c4cccc5oc6ccccc6c45)n3)cc2)cc1]

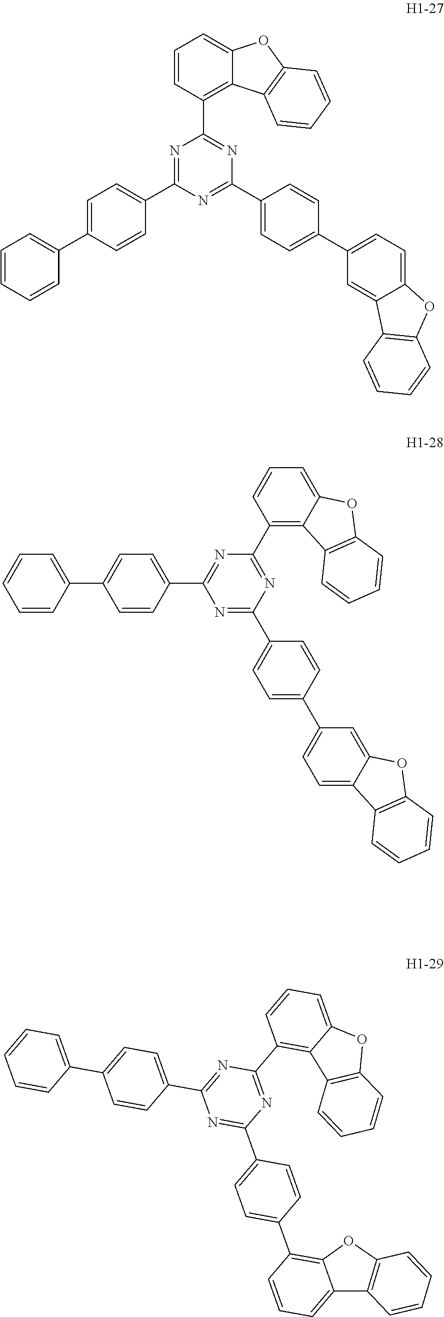 [SMILES: c1ccc(-c2ccc(-c3nc(-c4ccc(-c5ccc6oc7ccccc7c6c5)cc4)nc(-c4cccc5oc6ccccc6c45)n3)cc2)cc1 ; c1ccc(-c2ccc(-c3nc(-c4ccc(-c5ccc6c(c5)oc5ccccc56)cc4)nc(-c4cccc5oc6ccccc6c45)n3)cc2)cc1 ; c1ccc(-c2ccc(-c3nc(-c4ccc(-c5cccc6c5oc5ccccc56)cc4)nc(-c4cccc5oc6ccccc6c45)n3)cc2)cc1]

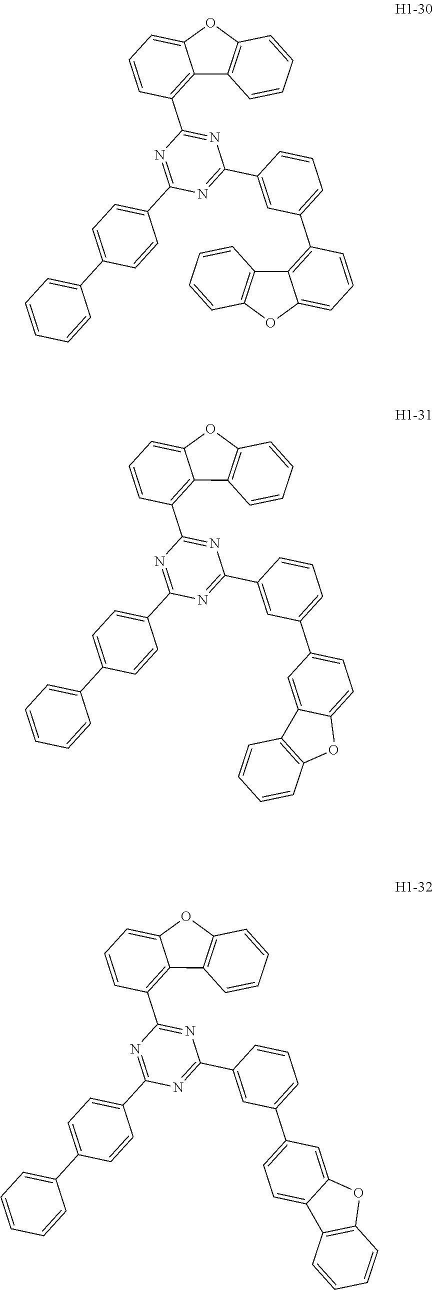 [SMILES: c1ccc(-c2ccc(-c3nc(-c4cccc(-c5cccc6oc7ccccc7c56)c4)nc(-c4cccc5oc6ccccc6c45)n3)cc2)cc1 ; c1ccc(-c2ccc(-c3nc(-c4cccc(-c5ccc6oc7ccccc7c6c5)c4)nc(-c4cccc5oc6ccccc6c45)n3)cc2)cc1 ; c1ccc(-c2ccc(-c3nc(-c4cccc(-c5ccc6c(c5)oc5ccccc56)c4)nc(-c4cccc5oc6ccccc6c45)n3)cc2)cc1]

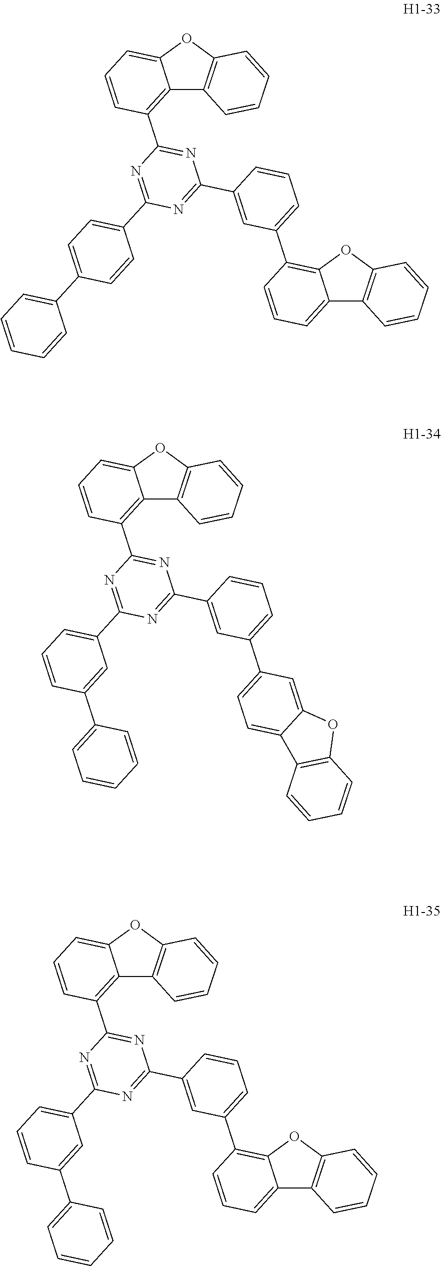 [SMILES: c1ccc(-c2ccc(-c3nc(-c4cccc(-c5cccc6c5oc5ccccc56)c4)nc(-c4cccc5oc6ccccc6c45)n3)cc2)cc1 ; c1ccc(-c2cccc(-c3nc(-c4cccc(-c5ccc6c(c5)oc5ccccc56)c4)nc(-c4cccc5oc6ccccc6c45)n3)c2)cc1 ; c1ccc(-c2cccc(-c3nc(-c4cccc(-c5cccc6c5oc5ccccc56)c4)nc(-c4cccc5oc6ccccc6c45)n3)c2)cc1]

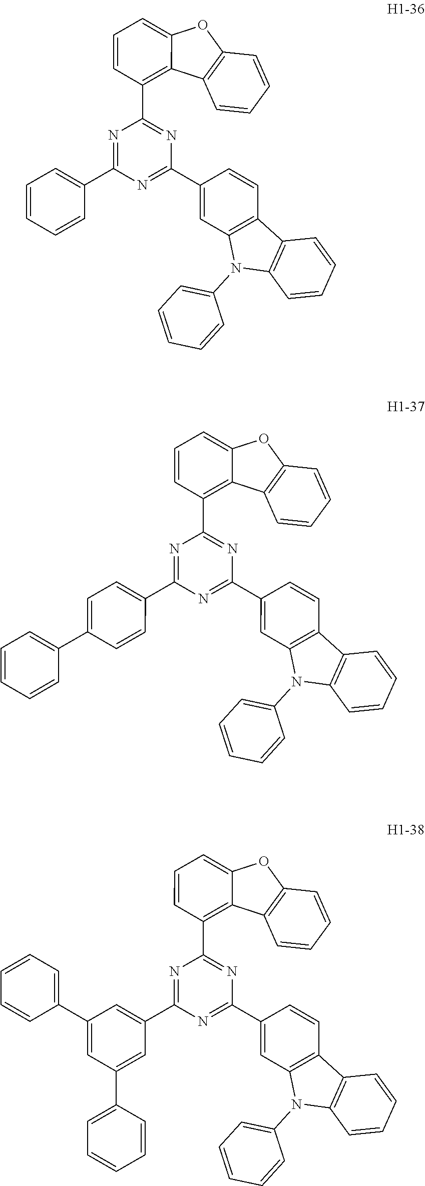 [SMILES: c1ccc(-c2nc(-c3ccc4c5ccccc5n(-c5ccccc5)c4c3)nc(-c3cccc4oc5ccccc5c34)n2)cc1 ; c1ccc(-c2ccc(-c3nc(-c4ccc5c6ccccc6n(-c6ccccc6)c5c4)nc(-c4cccc5oc6ccccc6c45)n3)cc2)cc1 ; c1ccc(-c2cc(-c3ccccc3)cc(-c3nc(-c4ccc5c6ccccc6n(-c6ccccc6)c5c4)nc(-c4cccc5oc6ccccc6c45)n3)c2)cc1]

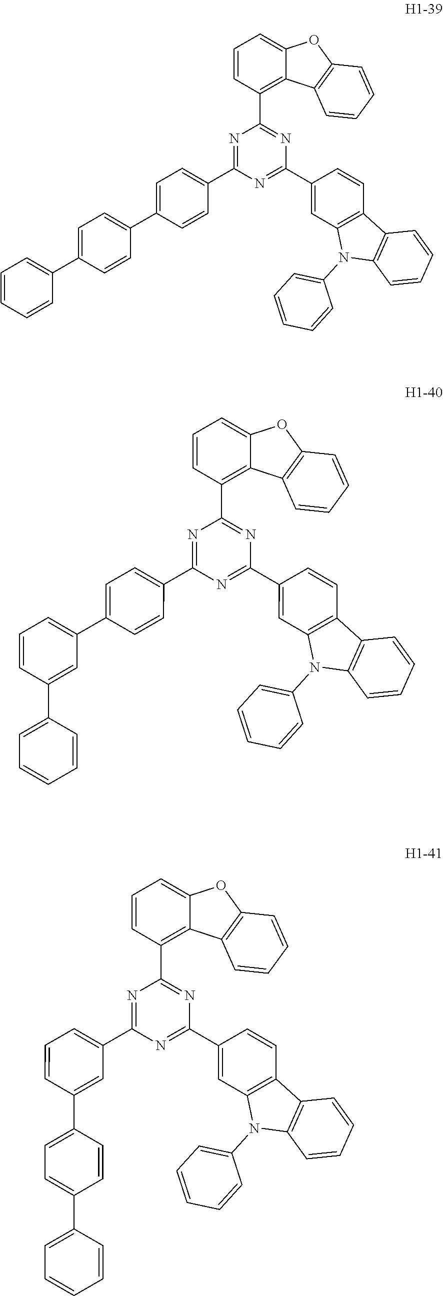 [SMILES: c1ccc(-c2ccc(-c3ccc(-c4nc(-c5ccc6c7ccccc7n(-c7ccccc7)c6c5)nc(-c5cccc6oc7ccccc7c56)n4)cc3)cc2)cc1 ; c1ccc(-c2cccc(-c3ccc(-c4nc(-c5ccc6c7ccccc7n(-c7ccccc7)c6c5)nc(-c5cccc6oc7ccccc7c56)n4)cc3)c2)cc1 ; c1ccc(-c2ccc(-c3cccc(-c4nc(-c5ccc6c7ccccc7n(-c7ccccc7)c6c5)nc(-c5cccc6oc7ccccc7c56)n4)c3)cc2)cc1]

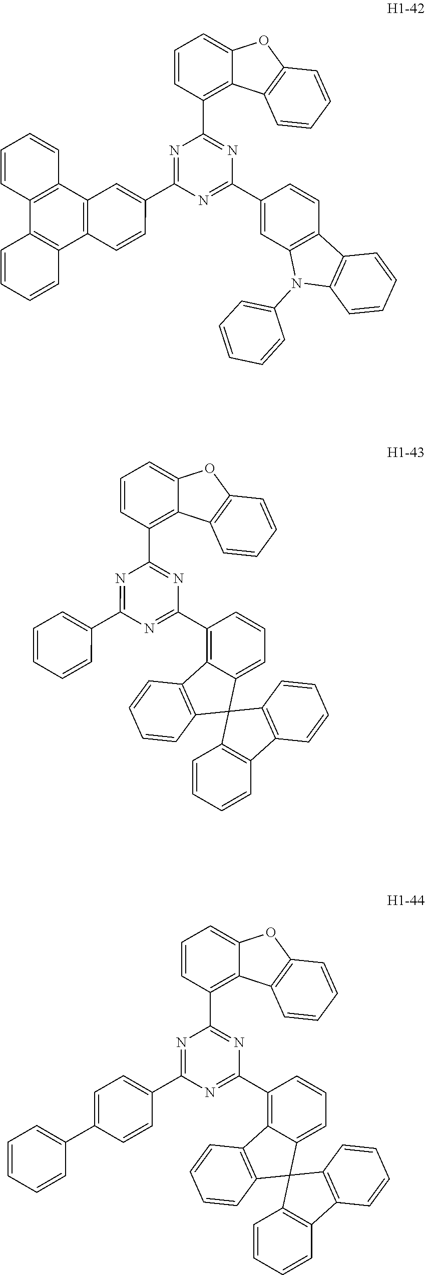 [SMILES: c1ccc(-n2c3ccccc3c3ccc(-c4nc(-c5ccc6c7ccccc7c7ccccc7c6c5)nc(-c5cccc6oc7ccccc7c56)n4)cc32)cc1 ; c1ccc(-c2nc(-c3cccc4c3-c3ccccc3C43c4ccccc4-c4ccccc43)nc(-c3cccc4oc5ccccc5c34)n2)cc1 ; c1ccc(-c2ccc(-c3nc(-c4cccc5c4-c4ccccc4C54c5ccccc5-c5ccccc54)nc(-c4cccc5oc6ccccc6c45)n3)cc2)cc1]

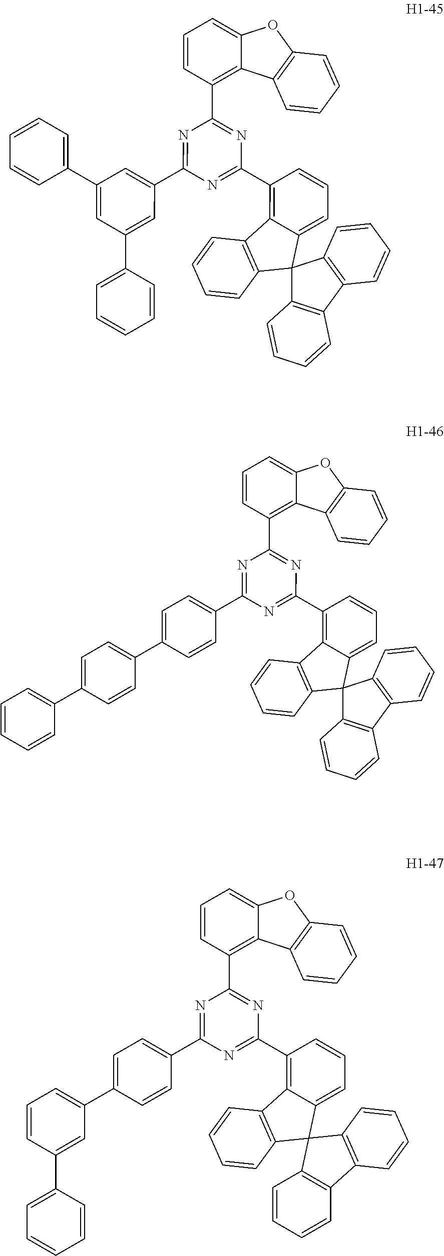 [SMILES: c1ccc(-c2cc(-c3ccccc3)cc(-c3nc(-c4cccc5c4-c4ccccc4C54c5ccccc5-c5ccccc54)nc(-c4cccc5oc6ccccc6c45)n3)c2)cc1 ; c1ccc(-c2ccc(-c3ccc(-c4nc(-c5cccc6c5-c5ccccc5C65c6ccccc6-c6ccccc65)nc(-c5cccc6oc7ccccc7c56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6oc7ccccc7c56)n4)cc3)c2)cc1]

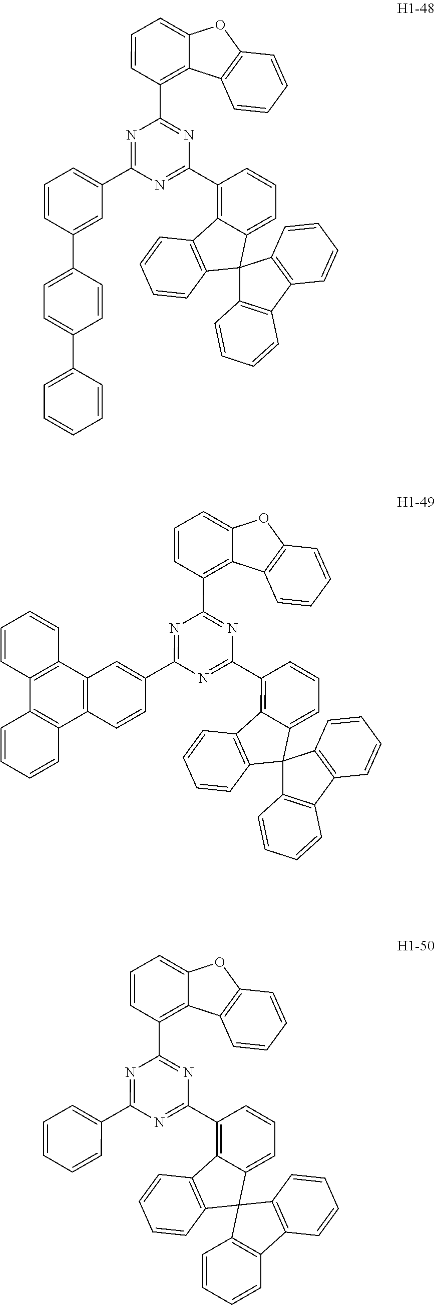 [SMILES: c1ccc(-c2ccc(-c3cccc(-c4nc(-c5cccc6c5-c5ccccc5C65c6ccccc6-c6ccccc65)nc(-c5cccc6oc7ccccc7c56)n4)c3)cc2)cc1 ; c1ccc2c(c1)-c1ccccc1C21c2ccccc2-c2c(-c3nc(-c4ccc5c6ccccc6c6ccccc6c5c4)nc(-c4cccc5oc6ccccc6c45)n3)cccc21 ; c1ccc(-c2nc(-c3cccc4c3-c3ccccc3C43c4ccccc4-c4ccccc43)nc(-c3cccc4oc5ccccc5c34)n2)cc1]

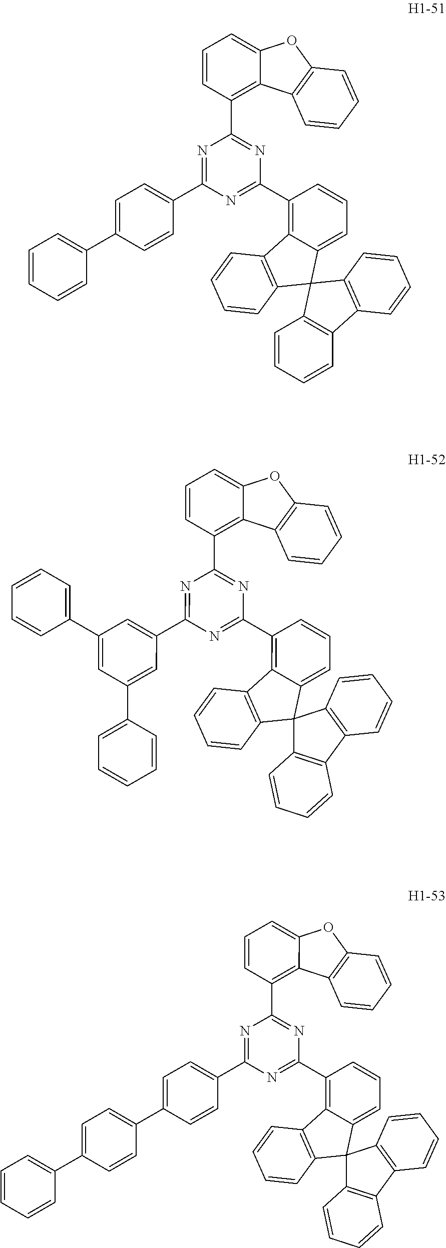 [SMILES: c1ccc(-c2ccc(-c3nc(-c4cccc5c4-c4ccccc4C54c5ccccc5-c5ccccc54)nc(-c4cccc5oc6ccccc6c45)n3)cc2)cc1 ; c1ccc(-c2cc(-c3ccccc3)cc(-c3nc(-c4cccc5c4-c4ccccc4C54c5ccccc5-c5ccccc54)nc(-c4cccc5oc6ccccc6c45)n3)c2)cc1 ; c1ccc(-c2ccc(-c3ccc(-c4nc(-c5cccc6c5-c5ccccc5C65c6ccccc6-c6ccccc65)nc(-c5cccc6oc7ccccc7c56)n4)cc3)cc2)cc1]

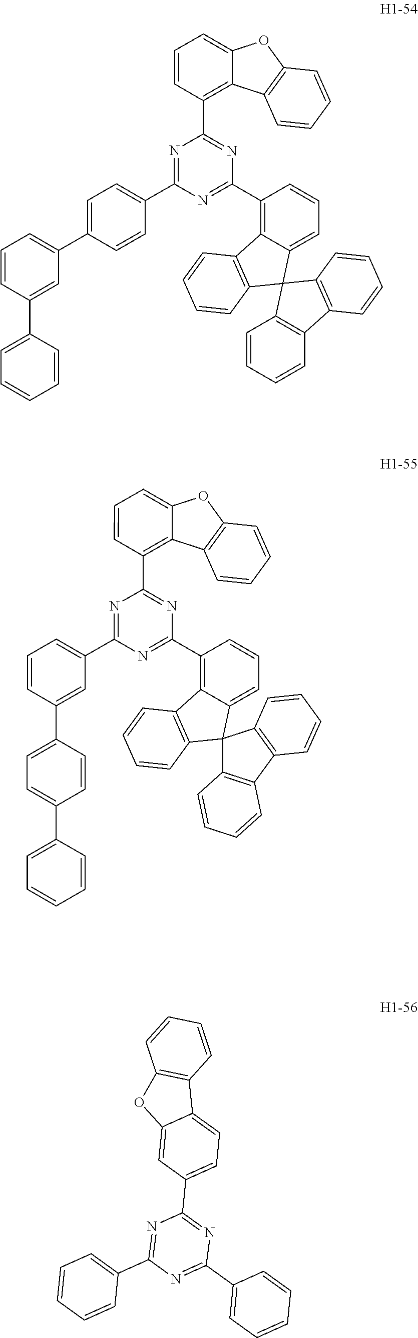 [SMILES: c1ccc(-c2cccc(-c3ccc(-c4nc(-c5cccc6c5-c5ccccc5C65c6ccccc6-c6ccccc65)nc(-c5cccc6oc7ccccc7c56)n4)cc3)c2)cc1 ; c1ccc(-c2ccc(-c3cccc(-c4nc(-c5cccc6c5-c5ccccc5C65c6ccccc6-c6ccccc65)nc(-c5cccc6oc7ccccc7c56)n4)c3)cc2)cc1 ; c1ccc(-c2nc(-c3ccccc3)nc(-c3ccc4c(c3)oc3ccccc34)n2)cc1]

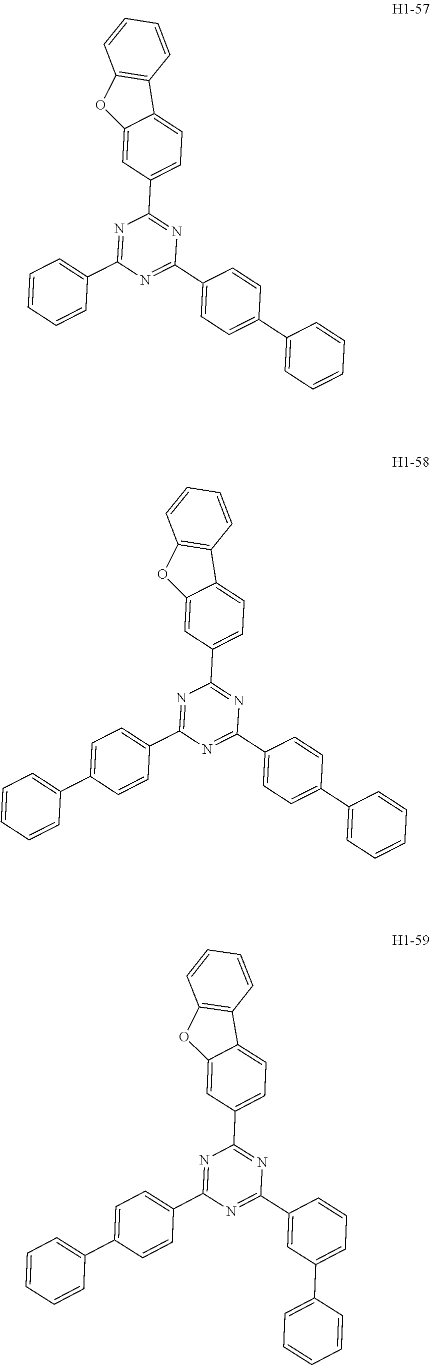 [SMILES: c1ccc(-c2ccc(-c3nc(-c4ccccc4)nc(-c4ccc5c(c4)oc4ccccc45)n3)cc2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4cccc(-c5ccccc5)c4)nc(-c4ccc5c(c4)oc4ccccc45)n3)cc2)cc1]

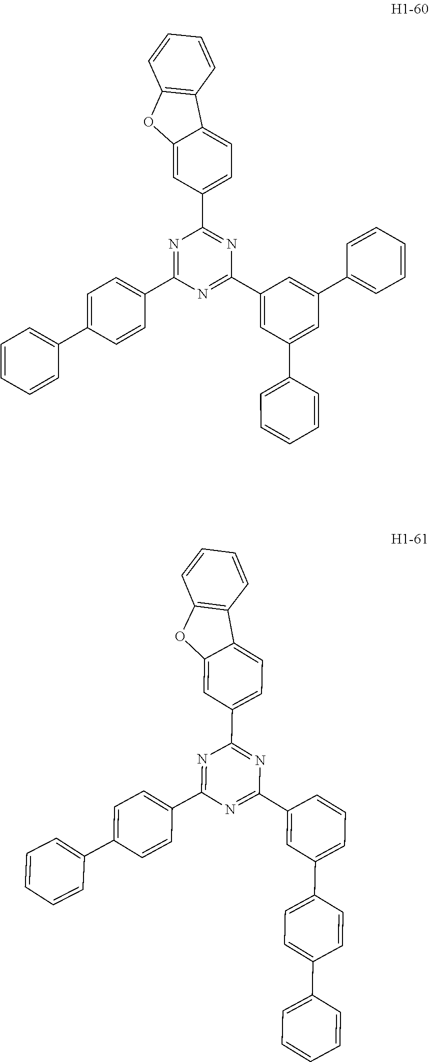 [SMILES: c1ccc(-c2ccc(-c3nc(-c4cc(-c5ccccc5)cc(-c5ccccc5)c4)nc(-c4ccc5c(c4)oc4ccccc45)n3)cc2)cc1 ; c1ccc(-c2ccc(-c3cccc(-c4nc(-c5ccc(-c6ccccc6)cc5)nc(-c5ccc6c(c5)oc5ccccc56)n4)c3)cc2)cc1]

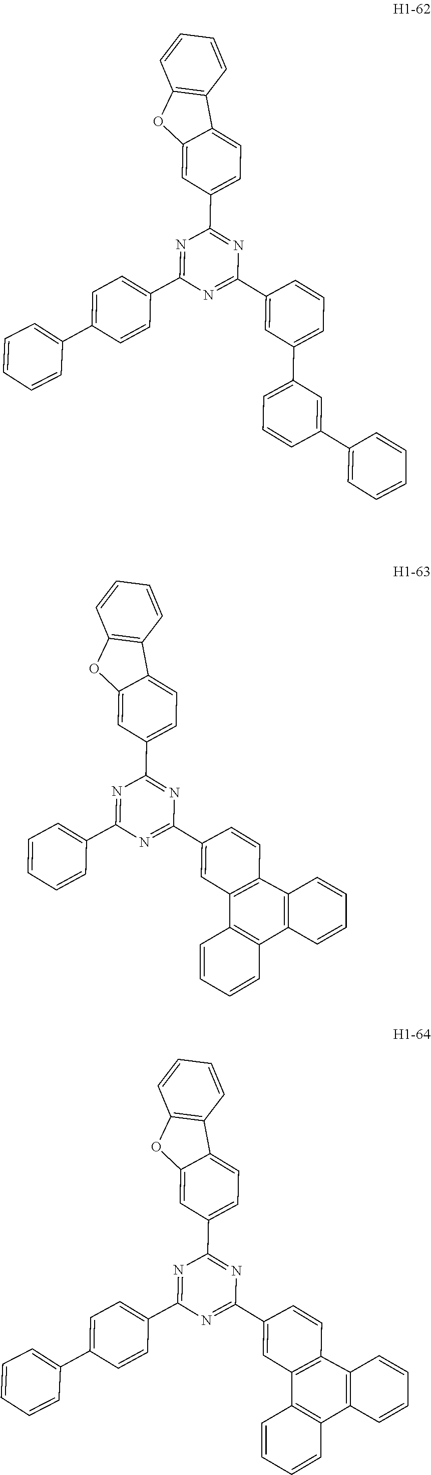 [SMILES: c1ccc(-c2ccc(-c3nc(-c4cccc(-c5cccc(-c6ccccc6)c5)c4)nc(-c4ccc5c(c4)oc4ccccc45)n3)cc2)cc1 ; c1ccc(-c2nc(-c3ccc4c(c3)oc3ccccc34)nc(-c3ccc4c5ccccc5c5ccccc5c4c3)n2)cc1 ; c1ccc(-c2ccc(-c3nc(-c4ccc5c(c4)oc4ccccc45)nc(-c4ccc5c6ccccc6c6ccccc6c5c4)n3)cc2)cc1]

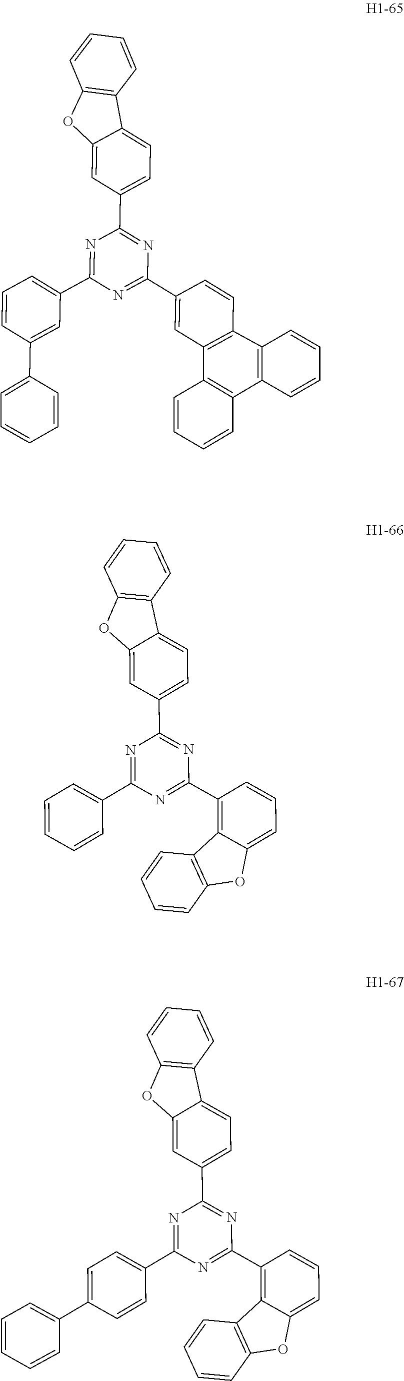 [SMILES: c1ccc(-c2cccc(-c3nc(-c4ccc5c(c4)oc4ccccc45)nc(-c4ccc5c6ccccc6c6ccccc6c5c4)n3)c2)cc1 ; c1ccc(-c2nc(-c3ccc4c(c3)oc3ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)cc2)cc1]

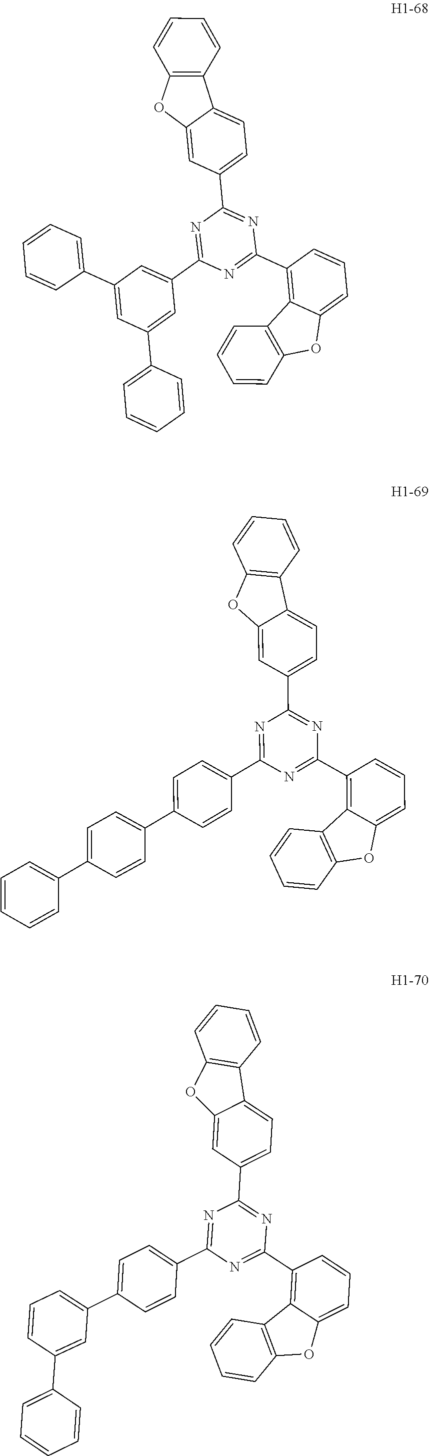 [SMILES: c1ccc(-c2cc(-c3ccccc3)cc(-c3nc(-c4ccc5c(c4)oc4ccccc45)nc(-c4cccc5oc6ccccc6c45)n3)c2)cc1 ; c1ccc(-c2ccc(-c3ccc(-c4nc(-c5ccc6c(c5)oc5ccccc56)nc(-c5cccc6oc7ccccc7c56)n4)cc3)cc2)cc1 ; c1ccc(-c2cccc(-c3ccc(-c4nc(-c5ccc6c(c5)oc5ccccc56)nc(-c5cccc6oc7ccccc7c56)n4)cc3)c2)cc1]

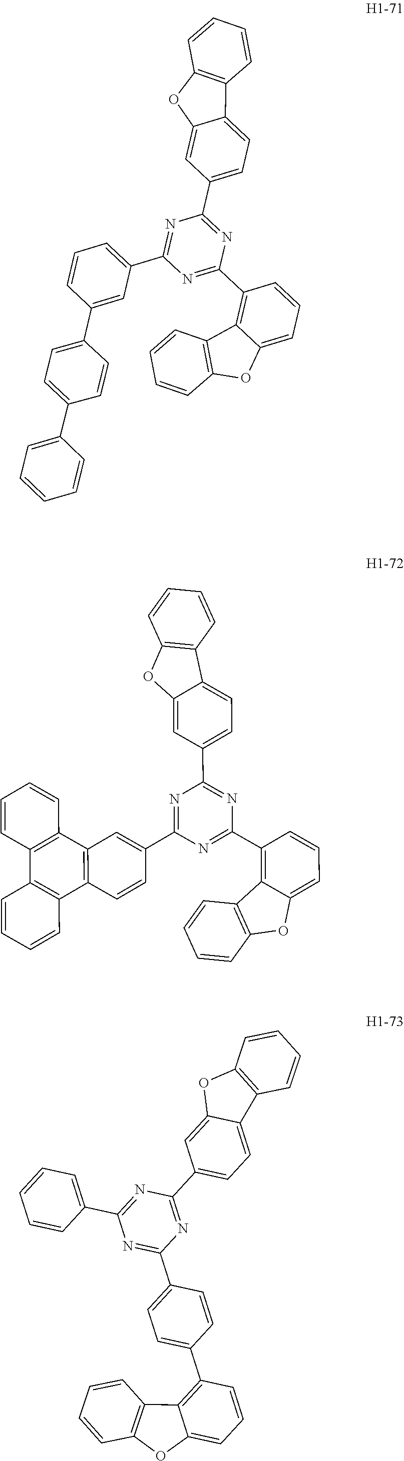 [SMILES: c1ccc(-c2ccc(-c3cccc(-c4nc(-c5ccc6c(c5)oc5ccccc56)nc(-c5cccc6oc7ccccc7c56)n4)c3)cc2)cc1 ; c1ccc2c(c1)oc1cc(-c3nc(-c4ccc5c6ccccc6c6ccccc6c5c4)nc(-c4cccc5oc6ccccc6c45)n3)ccc12 ; c1ccc(-c2nc(-c3ccc(-c4cccc5oc6ccccc6c45)cc3)nc(-c3ccc4c(c3)oc3ccccc34)n2)cc1]

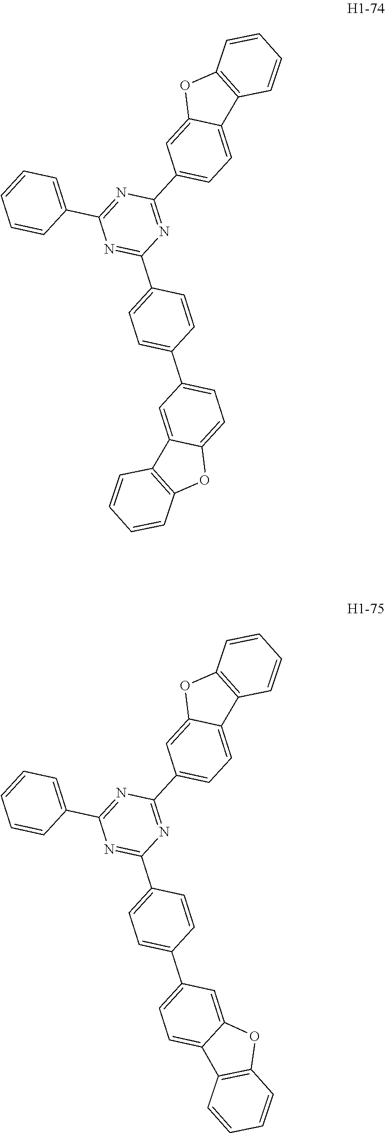 [SMILES: c1ccc(-c2nc(-c3ccc(-c4ccc5oc6ccccc6c5c4)cc3)nc(-c3ccc4c(c3)oc3ccccc34)n2)cc1 ; c1ccc(-c2nc(-c3ccc(-c4ccc5c(c4)oc4ccccc45)cc3)nc(-c3ccc4c(c3)oc3ccccc34)n2)cc1]

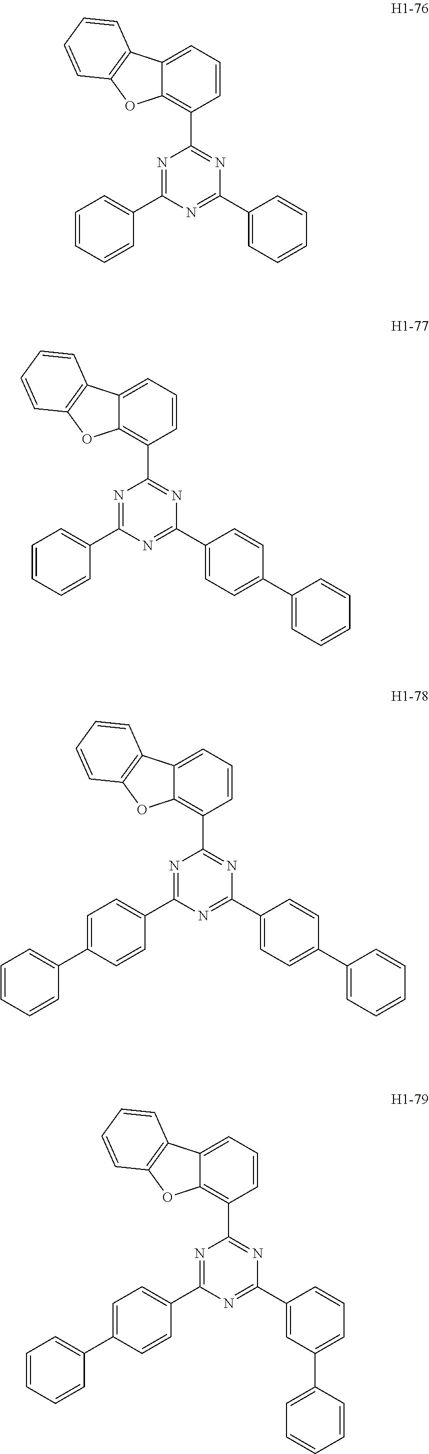 [SMILES: c1ccc(-c2nc(-c3ccccc3)nc(-c3cccc4c3oc3ccccc34)n2)cc1 ; c1ccc(-c2ccc(-c3nc(-c4ccccc4)nc(-c4cccc5c4oc4ccccc45)n3)cc2)cc1 ; c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4cccc(-c5ccccc5)c4)nc(-c4cccc5c4oc4ccccc45)n3)cc2)cc1]

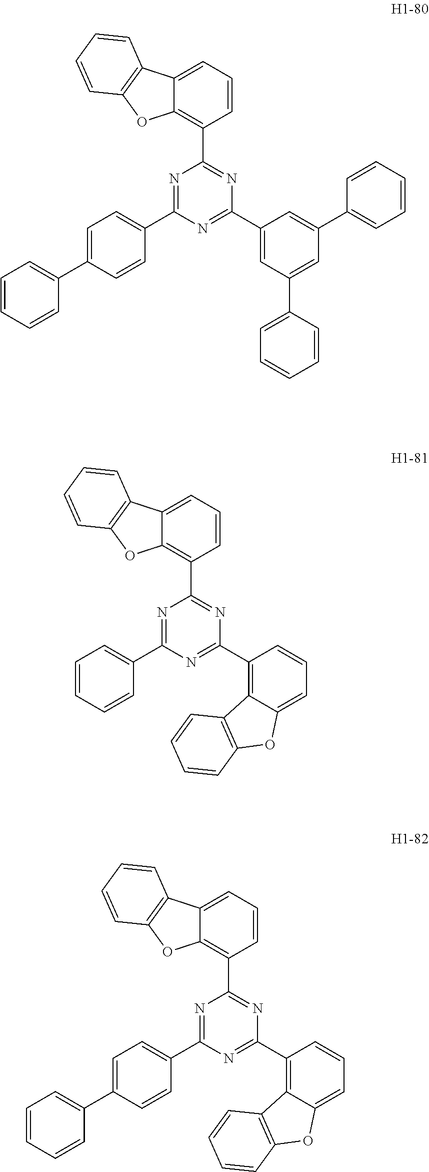 [SMILES: c1ccc(-c2ccc(-c3nc(-c4cc(-c5ccccc5)cc(-c5ccccc5)c4)nc(-c4cccc5c4oc4ccccc45)n3)cc2)cc1 ; c1ccc(-c2nc(-c3cccc4c3oc3ccccc34)nc(-c3cccc4oc5ccccc5c34)n2)cc1 ; c1ccc(-c2ccc(-c3nc(-c4cccc5c4oc4ccccc45)nc(-c4cccc5oc6ccccc6c45)n3)cc2)cc1]

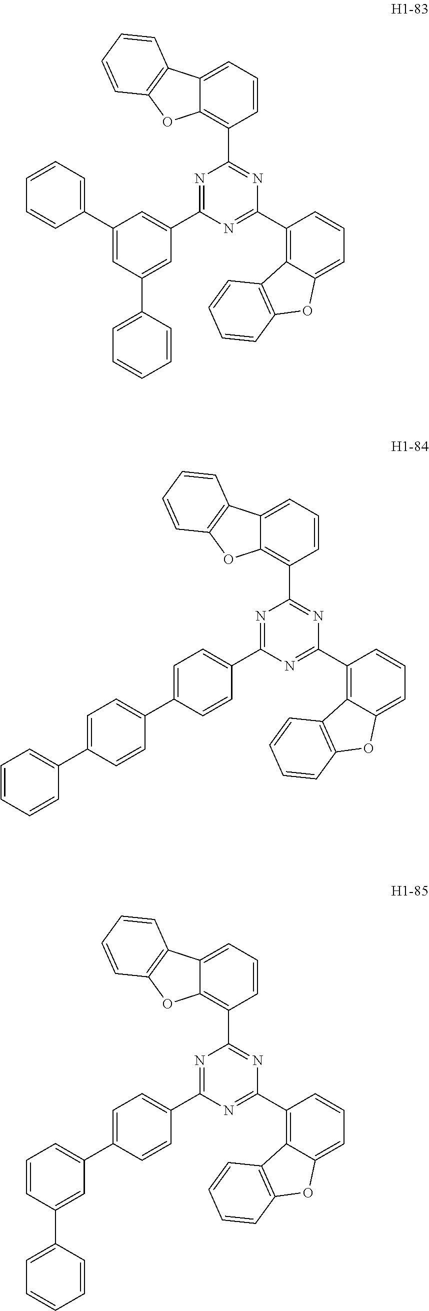 [SMILES: c1ccc(-c2cc(-c3ccccc3)cc(-c3nc(-c4cccc5c4oc4ccccc45)nc(-c4cccc5oc6ccccc6c45)n3)c2)cc1 ; c1ccc(-c2ccc(-c3ccc(-c4nc(-c5cccc6c5oc5ccccc56)nc(-c5cccc6oc7ccccc7c56)n4)cc3)cc2)cc1 ; c1ccc(-c2cccc(-c3ccc(-c4nc(-c5cccc6c5oc5ccccc56)nc(-c5cccc6oc7ccccc7c56)n4)cc3)c2)cc1]

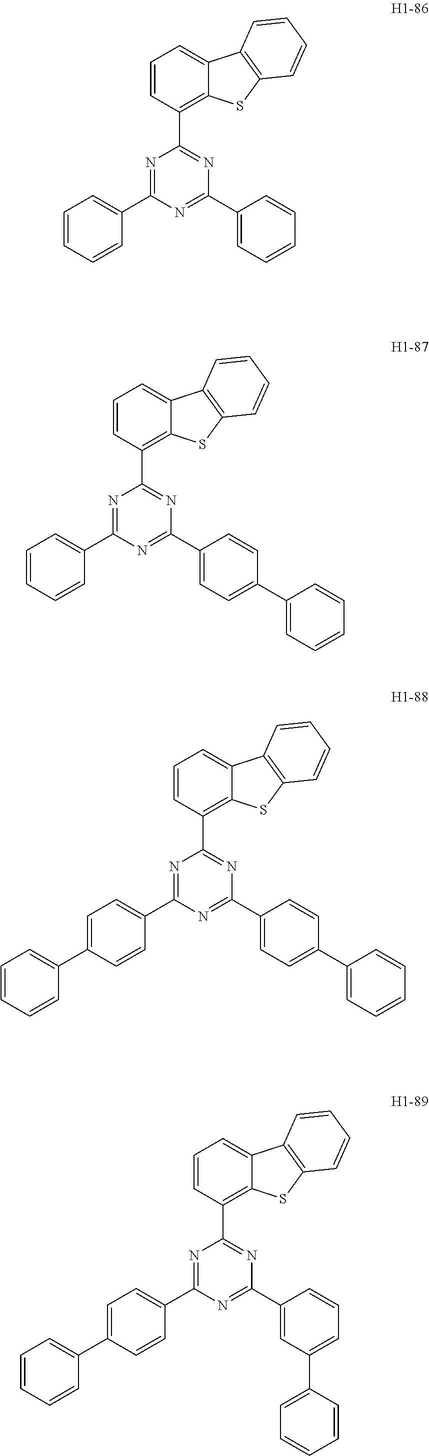 [SMILES: c1ccc(-c2nc(-c3ccccc3)nc(-c3cccc4c3sc3ccccc34)n2)cc1 ; c1ccc(-c2ccc(-c3nc(-c4ccccc4)nc(-c4cccc5c4sc4ccccc45)n3)cc2)cc1 ; c1ccc(-c2ccc(-c3nc(-c4ccc(-c5ccccc5)cc4)nc(-c4cccc5c4sc4ccccc45)n3)cc2)cc1 ; c1ccc(-c2ccc(-c3nc(-c4cccc(-c5ccccc5)c4)nc(-c4cccc5c4sc4ccccc45)n3)cc2)cc1]

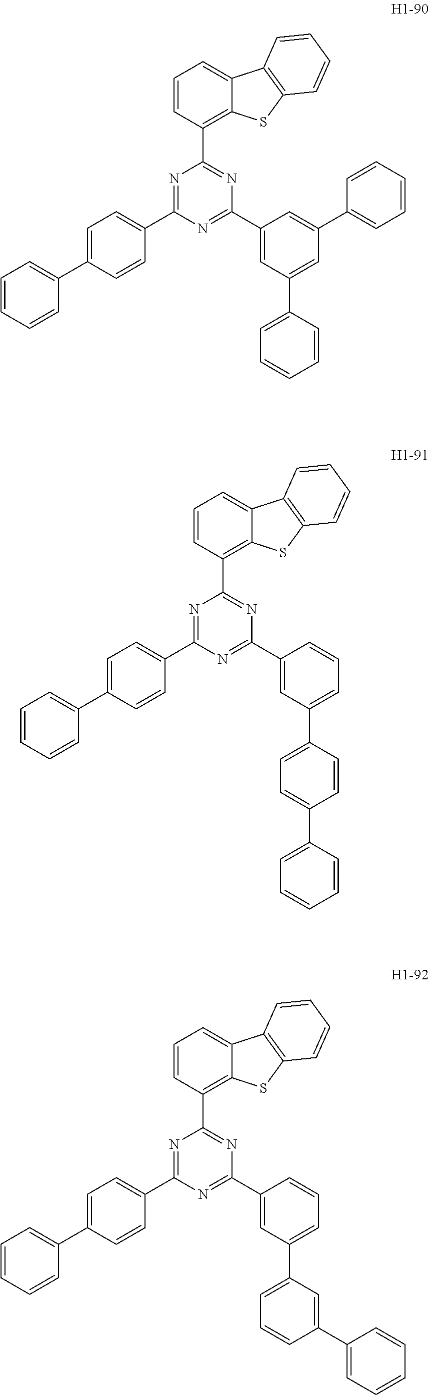 [SMILES: c1ccc(-c2ccc(-c3nc(-c4cc(-c5ccccc5)cc(-c5ccccc5)c4)nc(-c4cccc5c4sc4ccccc45)n3)cc2)cc1 ; c1ccc(-c2ccc(-c3cccc(-c4nc(-c5ccc(-c6ccccc6)cc5)nc(-c5cccc6c5sc5ccccc56)n4)c3)cc2)cc1 ; c1ccc(-c2ccc(-c3nc(-c4cccc(-c5cccc(-c6ccccc6)c5)c4)nc(-c4cccc5c4sc4ccccc45)n3)cc2)cc1]

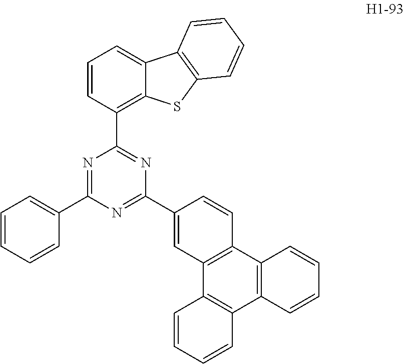 [SMILES: c1ccc(-c2nc(-c3ccc4c5ccccc5c5ccccc5c4c3)nc(-c3cccc4c3sc3ccccc34)n2)cc1]

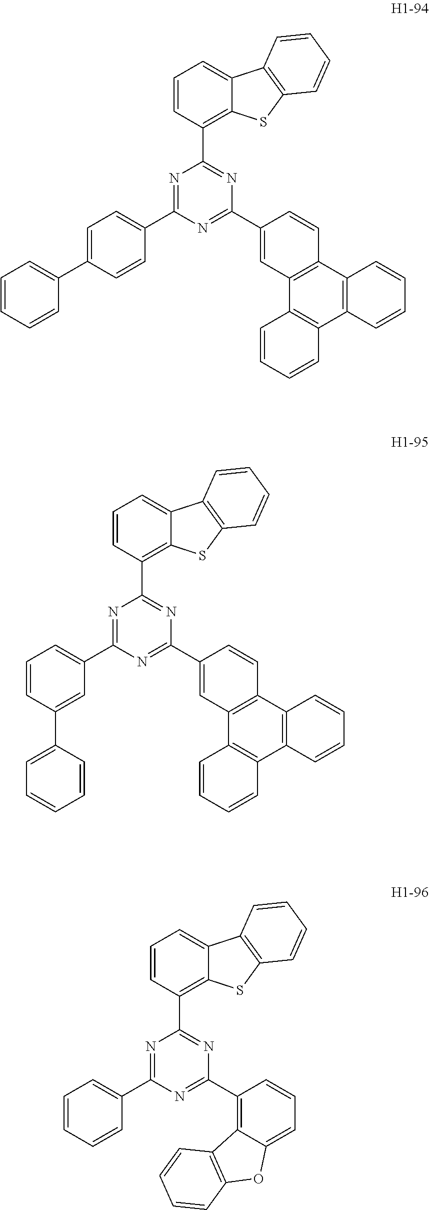 [SMILES: c1ccc(-c2ccc(-c3nc(-c4ccc5c6ccccc6c6ccccc6c5c4)nc(-c4cccc5c4sc4ccccc45)n3)cc2)cc1 ; c1ccc(-c2cccc(-c3nc(-c4ccc5c6ccccc6c6ccccc6c5c4)nc(-c4cccc5c4sc4ccccc45)n3)c2)cc1 ; c1ccc(-c2nc(-c3cccc4c3sc3ccccc34)nc(-c3cccc4oc5ccccc5c34)n2)cc1]

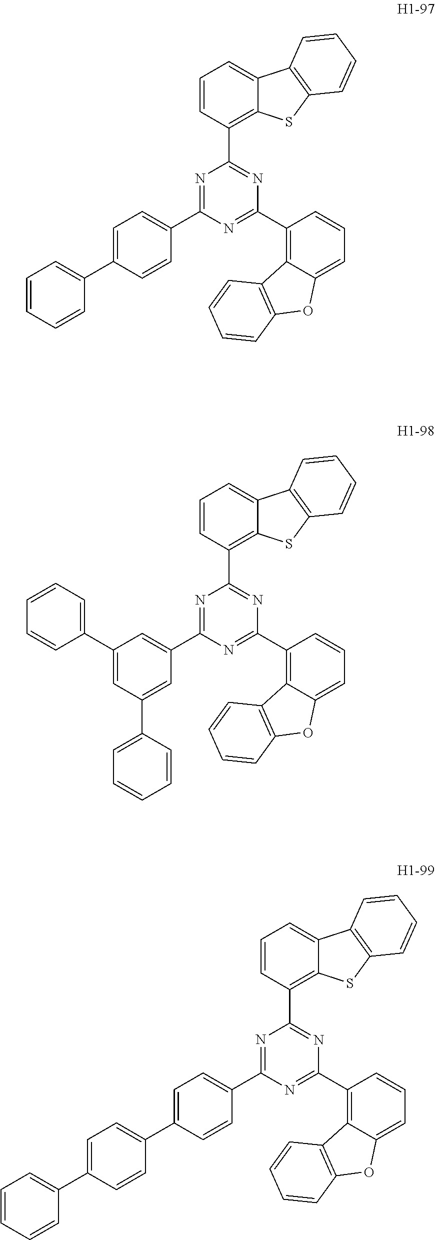 [SMILES: c1ccc(-c2ccc(-c3nc(-c4cccc5c4sc4ccccc45)nc(-c4cccc5oc6ccccc6c45)n3)cc2)cc1 ; c1ccc(-c2cc(-c3ccccc3)cc(-c3nc(-c4cccc5c4sc4ccccc45)nc(-c4cccc5oc6ccccc6c45)n3)c2)cc1 ; c1ccc(-c2ccc(-c3ccc(-c4nc(-c5cccc6c5sc5ccccc56)nc(-c5cccc6oc7ccccc7c56)n4)cc3)cc2)cc1]

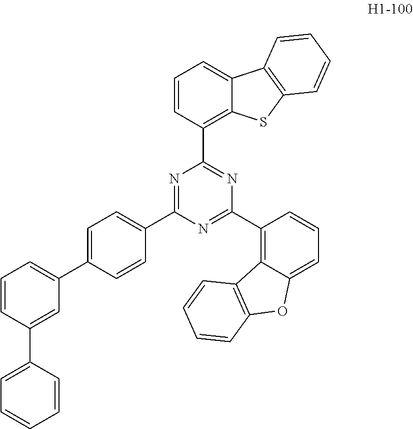 [SMILES: c1ccc(-c2cccc(-c3ccc(-c4nc(-c5cccc6c5sc5ccccc56)nc(-c5cccc6oc7ccccc7c56)n4)cc3)c2)cc1]

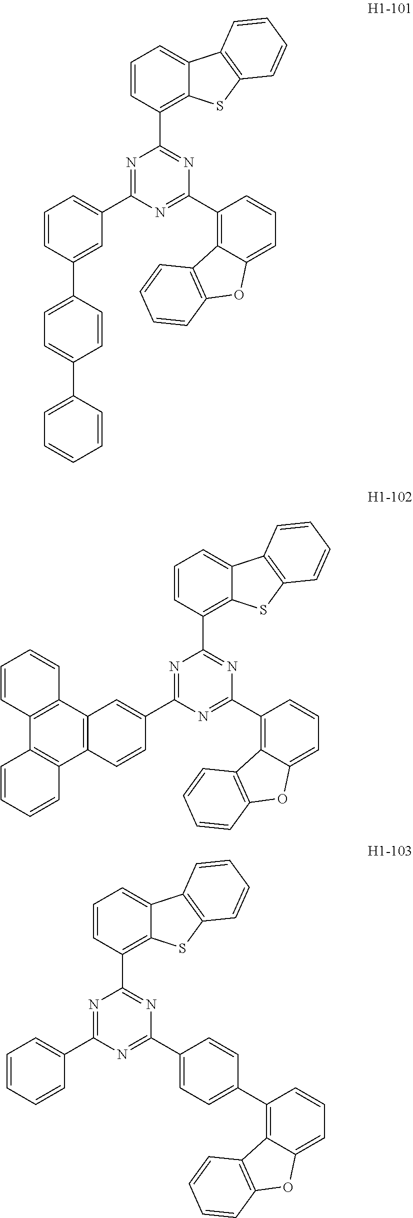

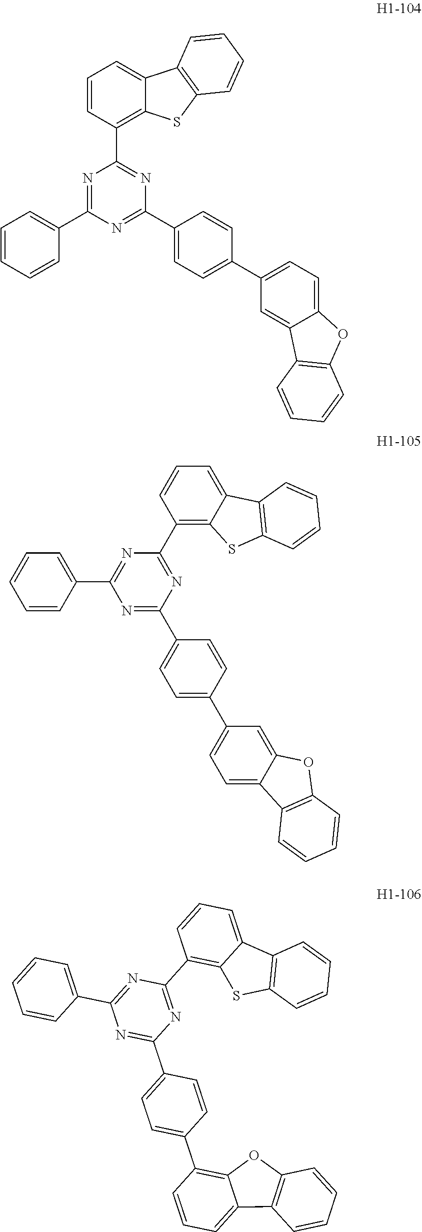 [SMILES: c1ccc(-c2nc(-c3ccc(-c4ccc5oc6ccccc6c5c4)cc3)nc(-c3cccc4c3sc3ccccc34)n2)cc1 ; c1ccc(-c2nc(-c3ccc(-c4ccc5c(c4)oc4ccccc45)cc3)nc(-c3cccc4c3sc3ccccc34)n2)cc1 ; c1ccc(-c2nc(-c3ccc(-c4cccc5c4oc4ccccc45)cc3)nc(-c3cccc4c3sc3ccccc34)n2)cc1]

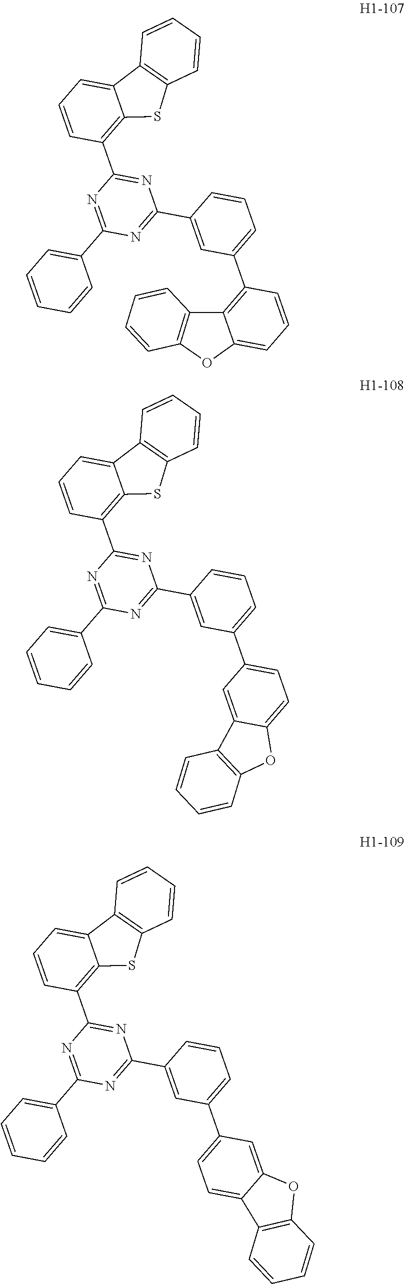 [SMILES: c1ccc(-c2nc(-c3cccc(-c4cccc5oc6ccccc6c45)c3)nc(-c3cccc4c3sc3ccccc34)n2)cc1 ; c1ccc(-c2nc(-c3cccc(-c4ccc5oc6ccccc6c5c4)c3)nc(-c3cccc4c3sc3ccccc34)n2)cc1 ; c1ccc(-c2nc(-c3cccc(-c4ccc5c(c4)oc4ccccc45)c3)nc(-c3cccc4c3sc3ccccc34)n2)cc1]

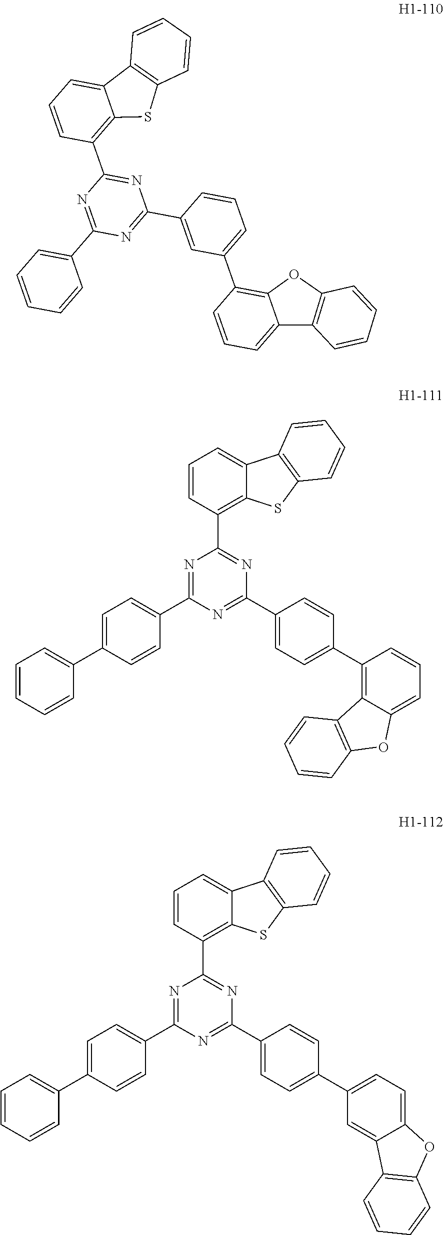 [SMILES: c1ccc(-c2nc(-c3cccc(-c4cccc5c4oc4ccccc45)c3)nc(-c3cccc4c3sc3ccccc34)n2)cc1 ; c1ccc(-c2ccc(-c3nc(-c4ccc(-c5cccc6oc7ccccc7c56)cc4)nc(-c4cccc5c4sc4ccccc45)n3)cc2)cc1 ; c1ccc(-c2ccc(-c3nc(-c4ccc(-c5ccc6oc7ccccc7c6c5)cc4)nc(-c4cccc5c4sc4ccccc45)n3)cc2)cc1]

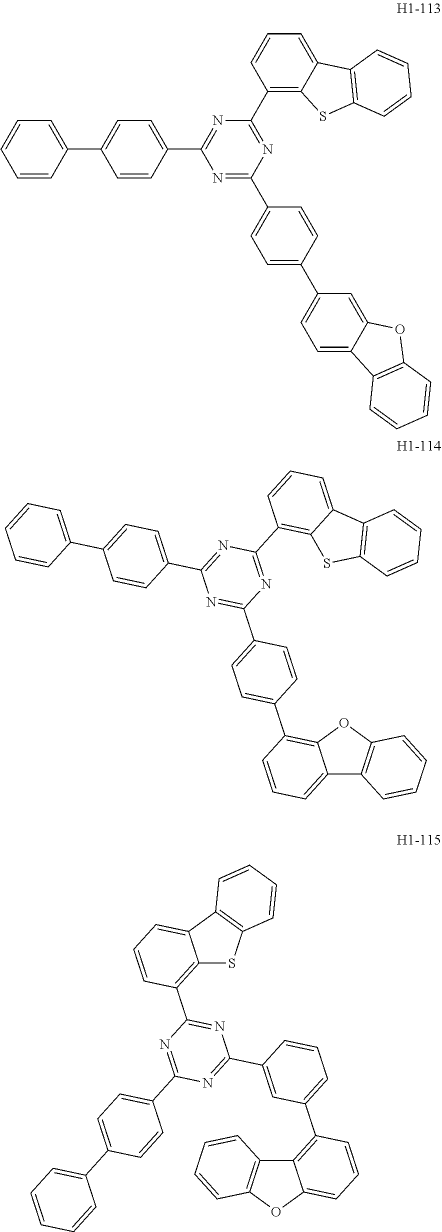 [SMILES: c1ccc(-c2ccc(-c3nc(-c4ccc(-c5ccc6c(c5)oc5ccccc56)cc4)nc(-c4cccc5c4sc4ccccc45)n3)cc2)cc1 ; c1ccc(-c2ccc(-c3nc(-c4ccc(-c5cccc6c5oc5ccccc56)cc4)nc(-c4cccc5c4sc4ccccc45)n3)cc2)cc1 ; c1ccc(-c2ccc(-c3nc(-c4cccc(-c5cccc6oc7ccccc7c56)c4)nc(-c4cccc5c4sc4ccccc45)n3)cc2)cc1]

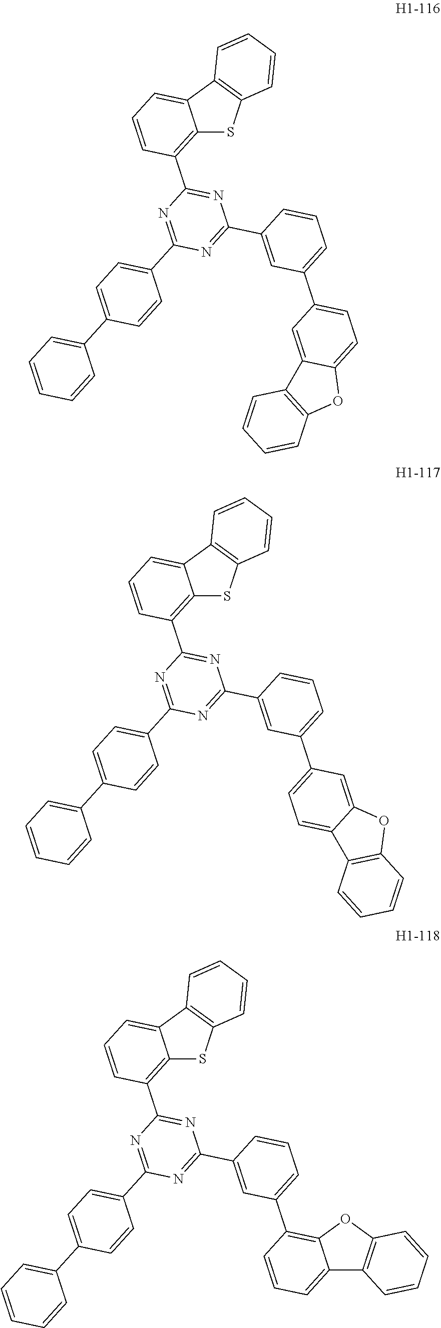 [SMILES: c1ccc(-c2ccc(-c3nc(-c4cccc(-c5ccc6oc7ccccc7c6c5)c4)nc(-c4cccc5c4sc4ccccc45)n3)cc2)cc1 ; c1ccc(-c2ccc(-c3nc(-c4cccc(-c5ccc6c(c5)oc5ccccc56)c4)nc(-c4cccc5c4sc4ccccc45)n3)cc2)cc1 ; c1ccc(-c2ccc(-c3nc(-c4cccc(-c5cccc6c5oc5ccccc56)c4)nc(-c4cccc5c4sc4ccccc45)n3)cc2)cc1]

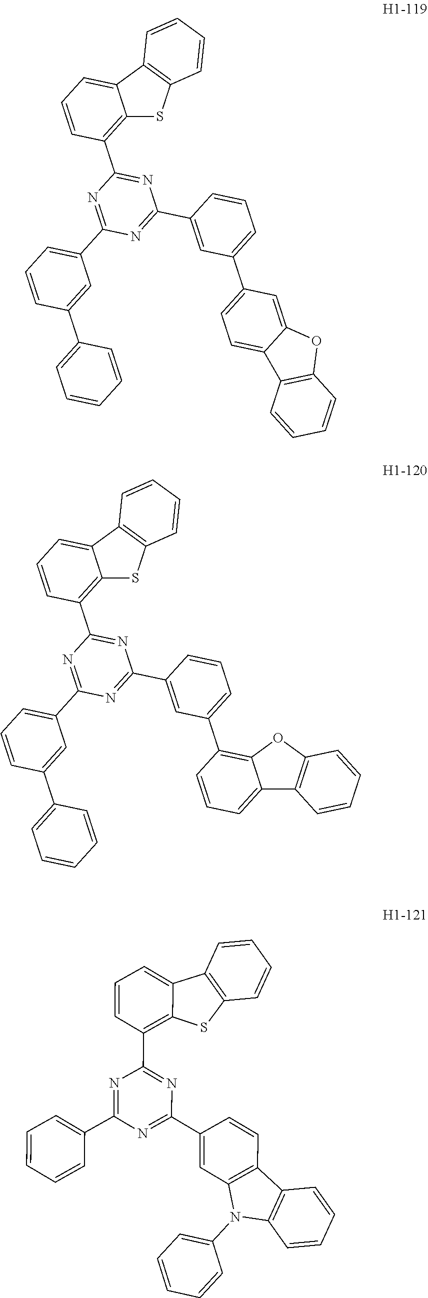 [SMILES: c1ccc(-c2cccc(-c3nc(-c4cccc(-c5ccc6c(c5)oc5ccccc56)c4)nc(-c4cccc5c4sc4ccccc45)n3)c2)cc1 ; c1ccc(-c2cccc(-c3nc(-c4cccc(-c5cccc6c5oc5ccccc56)c4)nc(-c4cccc5c4sc4ccccc45)n3)c2)cc1 ; c1ccc(-c2nc(-c3ccc4c5ccccc5n(-c5ccccc5)c4c3)nc(-c3cccc4c3sc3ccccc34)n2)cc1]

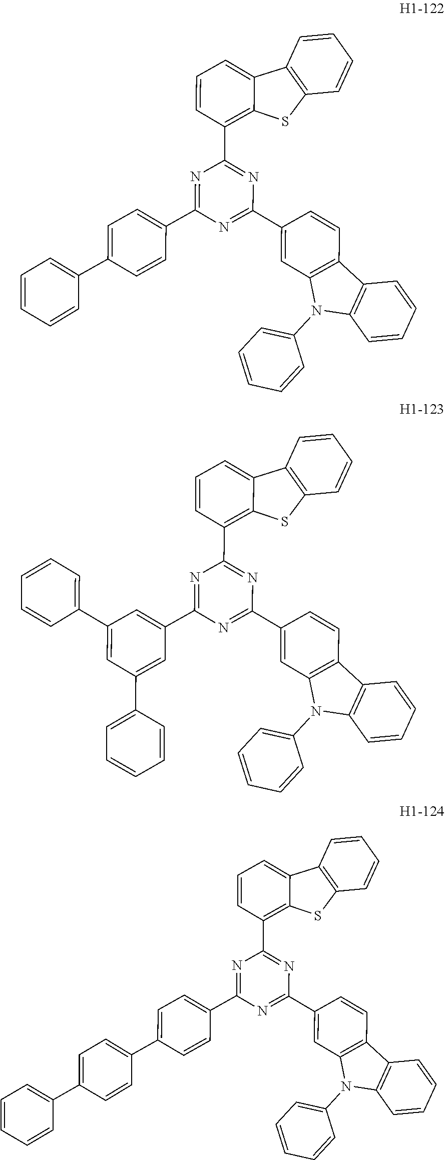 [SMILES: c1ccc(-c2ccc(-c3nc(-c4ccc5c6ccccc6n(-c6ccccc6)c5c4)nc(-c4cccc5c4sc4ccccc45)n3)cc2)cc1 ; c1ccc(-c2cc(-c3ccccc3)cc(-c3nc(-c4ccc5c6ccccc6n(-c6ccccc6)c5c4)nc(-c4cccc5c4sc4ccccc45)n3)c2)cc1 ; c1ccc(-c2ccc(-c3ccc(-c4nc(-c5ccc6c7ccccc7n(-c7ccccc7)c6c5)nc(-c5cccc6c5sc5ccccc56)n4)cc3)cc2)cc1]

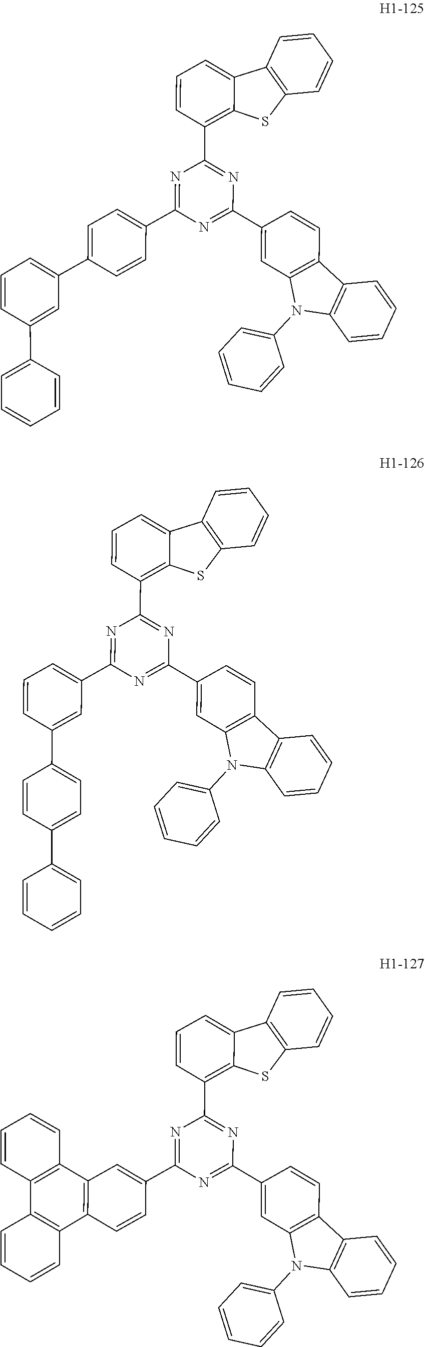 [SMILES: c1ccc(-c2cccc(-c3ccc(-c4nc(-c5ccc6c7ccccc7n(-c7ccccc7)c6c5)nc(-c5cccc6c5sc5ccccc56)n4)cc3)c2)cc1 ; c1ccc(-c2ccc(-c3cccc(-c4nc(-c5ccc6c7ccccc7n(-c7ccccc7)c6c5)nc(-c5cccc6c5sc5ccccc56)n4)c3)cc2)cc1 ; c1ccc(-n2c3ccccc3c3ccc(-c4nc(-c5ccc6c7ccccc7c7ccccc7c6c5)nc(-c5cccc6c5sc5ccccc56)n4)cc32)cc1]

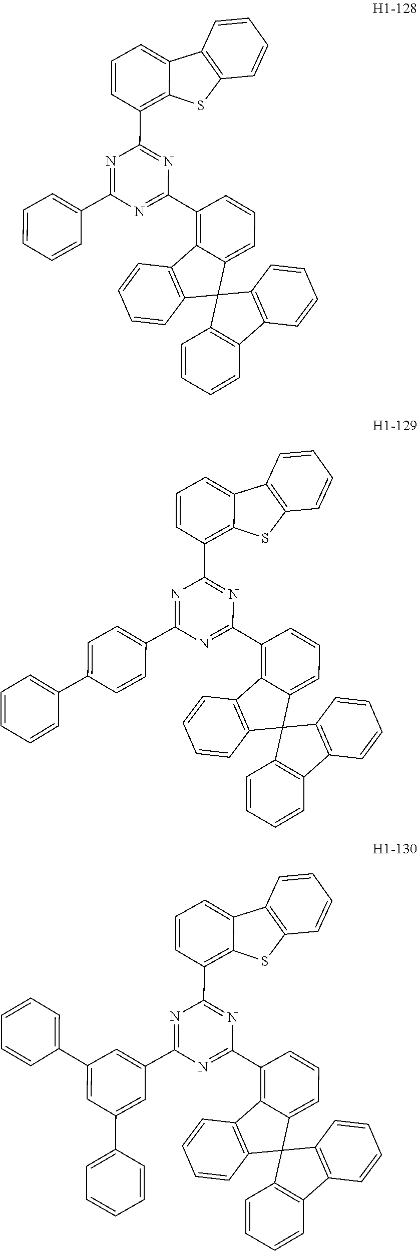 [SMILES: c1ccc(-c2nc(-c3cccc4c3-c3ccccc3C43c4ccccc4-c4ccccc43)nc(-c3cccc4c3sc3ccccc34)n2)cc1 ; c1ccc(-c2ccc(-c3nc(-c4cccc5c4-c4ccccc4C54c5ccccc5-c5ccccc54)nc(-c4cccc5c4sc4ccccc45)n3)cc2)cc1 ; c1ccc(-c2cc(-c3ccccc3)cc(-c3nc(-c4cccc5c4-c4ccccc4C54c5ccccc5-c5ccccc54)nc(-c4cccc5c4sc4ccccc45)n3)c2)cc1]

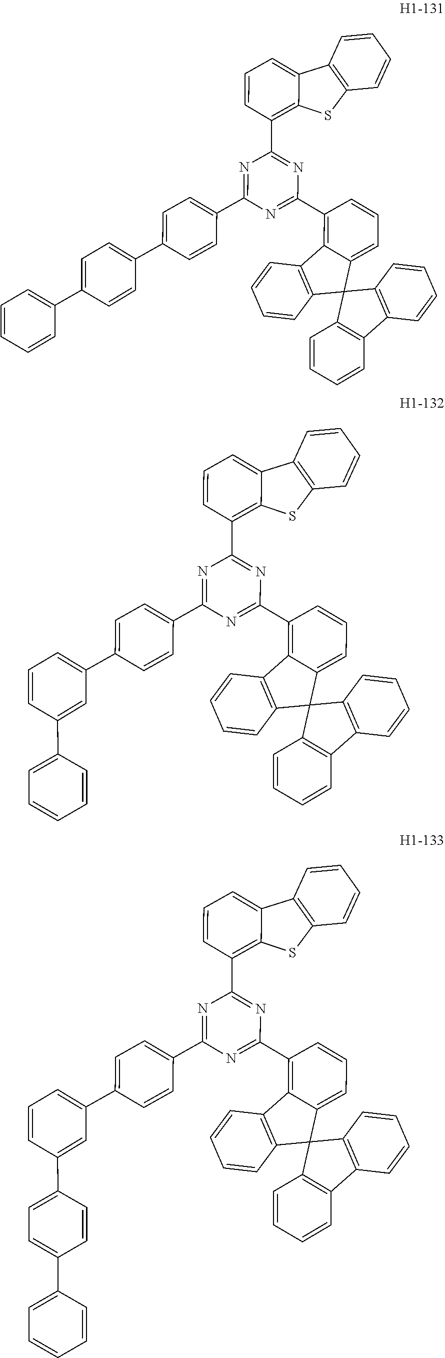

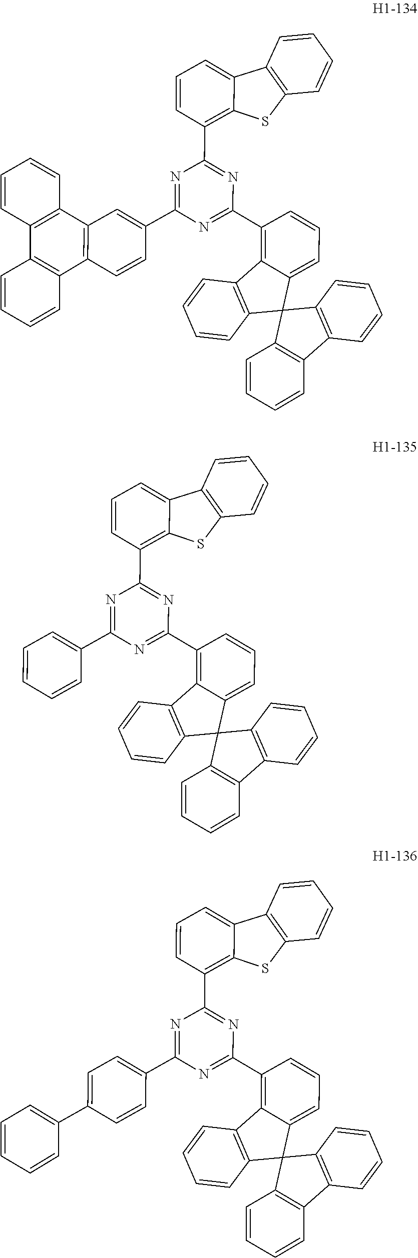 [SMILES: c1ccc2c(c1)-c1ccccc1C21c2ccccc2-c2c(-c3nc(-c4ccc5c6ccccc6c6ccccc6c5c4)nc(-c4cccc5c4sc4ccccc45)n3)cccc21 ; c1ccc(-c2nc(-c3cccc4c3-c3ccccc3C43c4ccccc4-c4ccccc43)nc(-c3cccc4c3sc3ccccc34)n2)cc1 ; c1ccc(-c2ccc(-c3nc(-c4cccc5c4-c4ccccc4C54c5ccccc5-c5ccccc54)nc(-c4cccc5c4sc4ccccc45)n3)cc2)cc1]

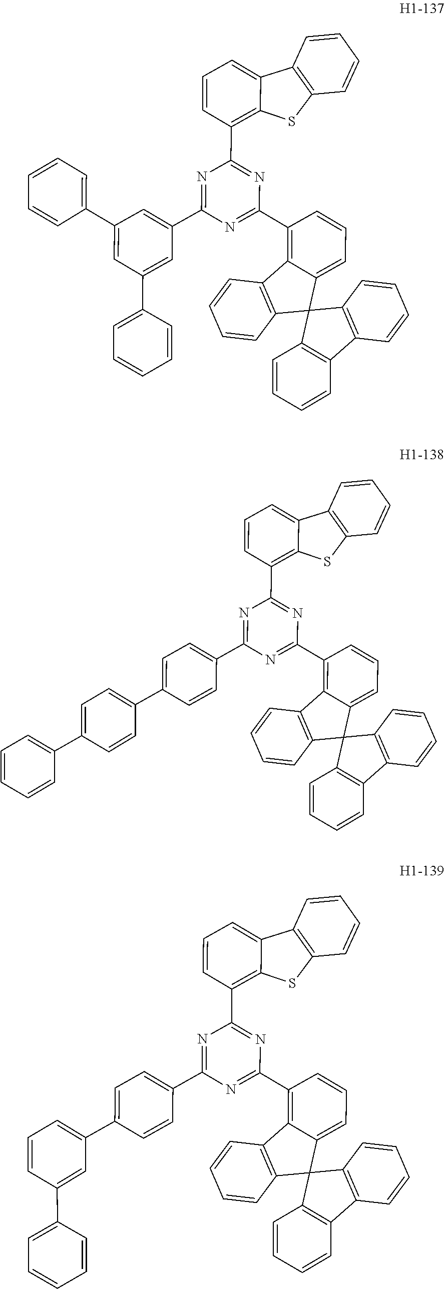 [SMILES: c1ccc(-c2cc(-c3ccccc3)cc(-c3nc(-c4cccc5c4-c4ccccc4C54c5ccccc5-c5ccccc54)nc(-c4cccc5c4sc4ccccc45)n3)c2)cc1 ; c1ccc(-c2ccc(-c3ccc(-c4nc(-c5cccc6c5-c5ccccc5C65c6ccccc6-c6ccccc65)nc(-c5cccc6c5sc5ccccc56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6c5sc5ccccc56)n4)cc3)c2)cc1]

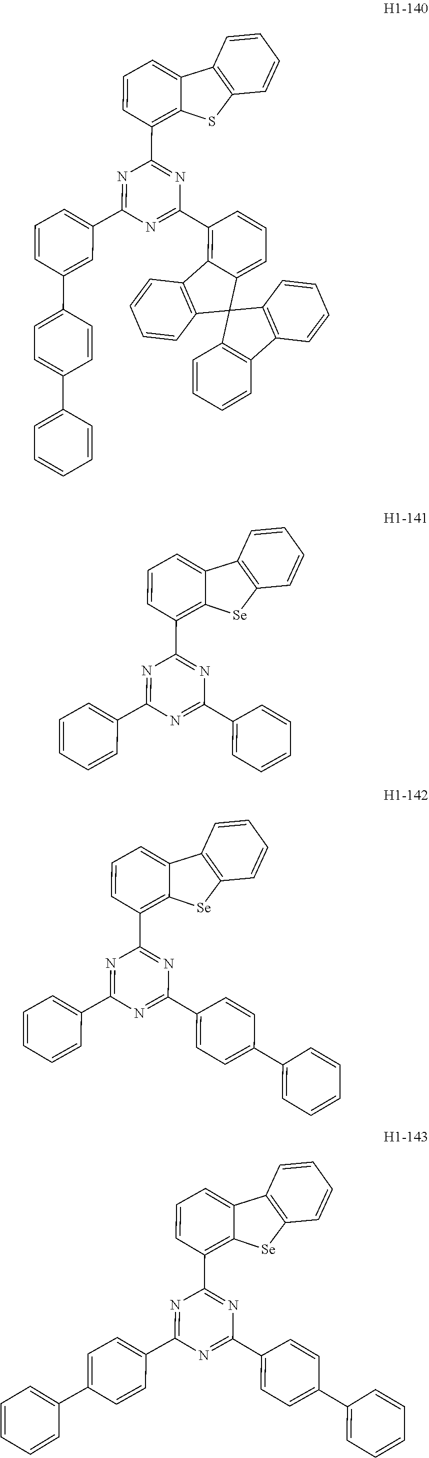

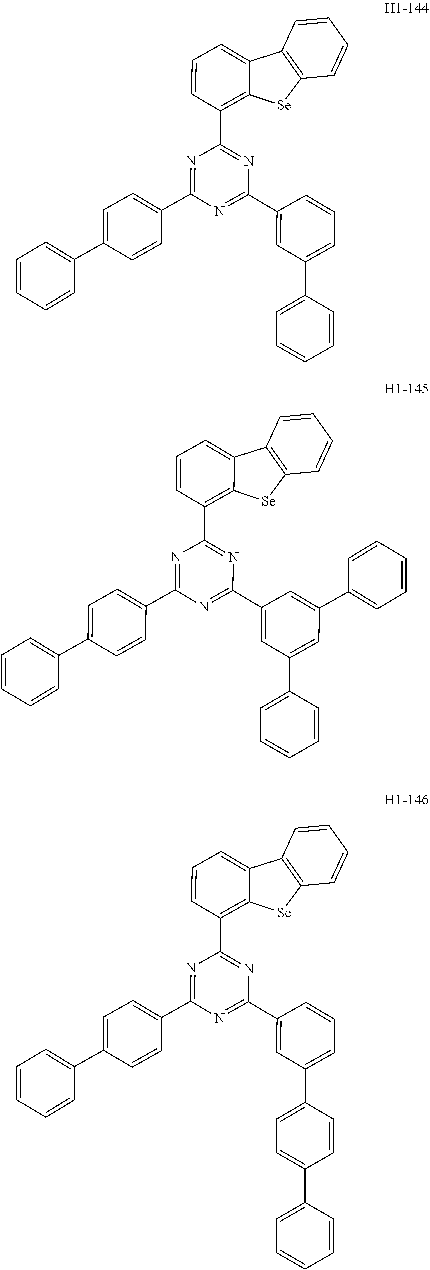 [SMILES: c1ccc(-c2ccc(-c3nc(-c4cccc(-c5ccccc5)c4)nc(-c4cccc5c4[se]c4ccccc45)n3)cc2)cc1 ; c1ccc(-c2ccc(-c3nc(-c4cc(-c5ccccc5)cc(-c5ccccc5)c4)nc(-c4cccc5c4[se]c4ccccc45)n3)cc2)cc1 ; c1ccc(-c2ccc(-c3cccc(-c4nc(-c5ccc(-c6ccccc6)cc5)nc(-c5cccc6c5[se]c5ccccc56)n4)c3)cc2)cc1]

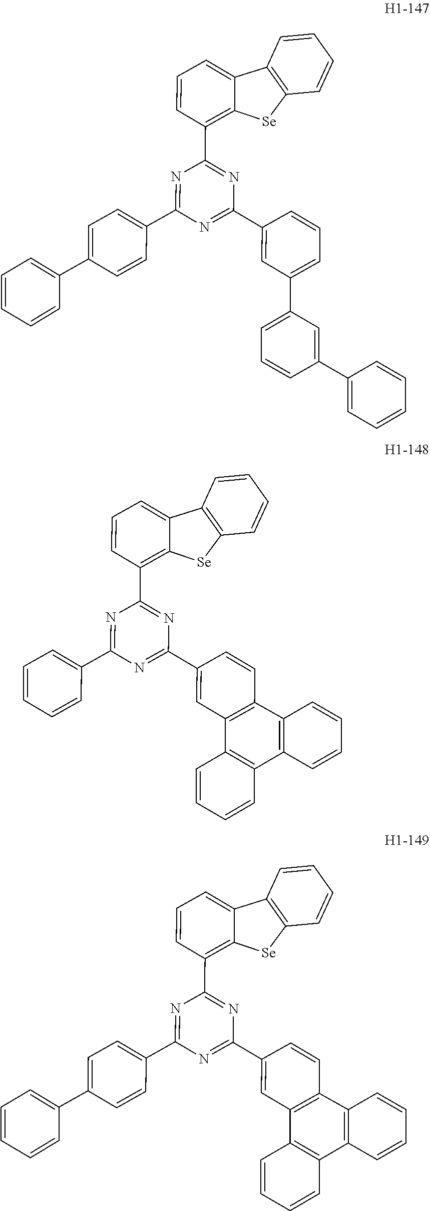 [SMILES: c1ccc(-c2ccc(-c3nc(-c4cccc(-c5cccc(-c6ccccc6)c5)c4)nc(-c4cccc5c4[se]c4ccccc45)n3)cc2)cc1 ; c1ccc(-c2nc(-c3ccc4c5ccccc5c5ccccc5c4c3)nc(-c3cccc4c3[se]c3ccccc34)n2)cc1 ; c1ccc(-c2ccc(-c3nc(-c4ccc5c6ccccc6c6ccccc6c5c4)nc(-c4cccc5c4[se]c4ccccc45)n3)cc2)cc1]

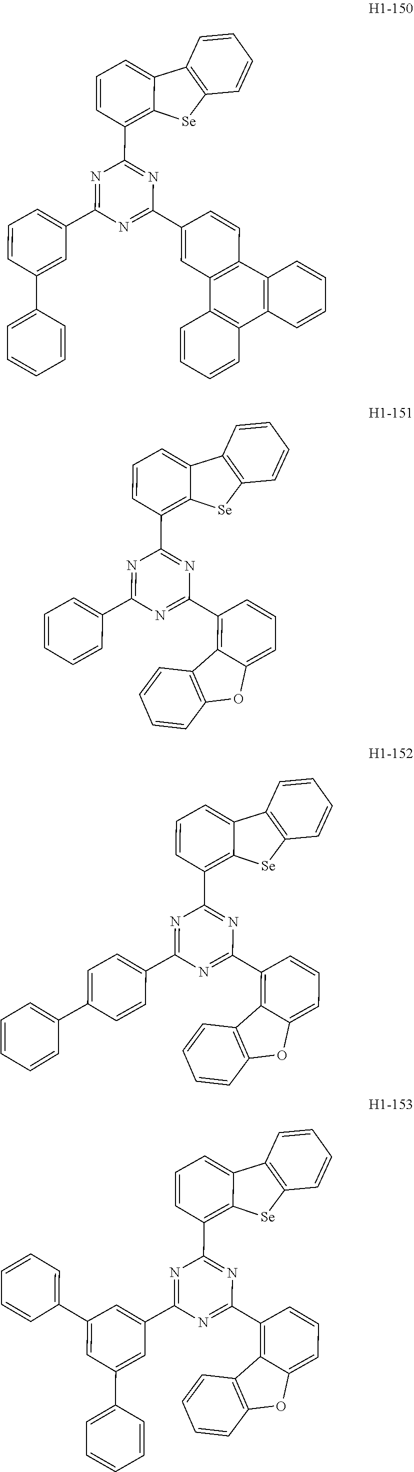 [SMILES: c1ccc(-c2cccc(-c3nc(-c4ccc5c6ccccc6c6ccccc6c5c4)nc(-c4cccc5c4[se]c4ccccc45)n3)c2)cc1 ; c1ccc(-c2nc(-c3cccc4c3[se]c3ccccc34)nc(-c3cccc4oc5ccccc5c34)n2)cc1 ; c1ccc(-c2ccc(-c3nc(-c4cccc5c4[se]c4ccccc45)nc(-c4cccc5oc6ccccc6c45)n3)cc2)cc1 ; c1ccc(-c2cc(-c3ccccc3)cc(-c3nc(-c4cccc5c4[se]c4ccccc45)nc(-c4cccc5oc6ccccc6c45)n3)c2)cc1]

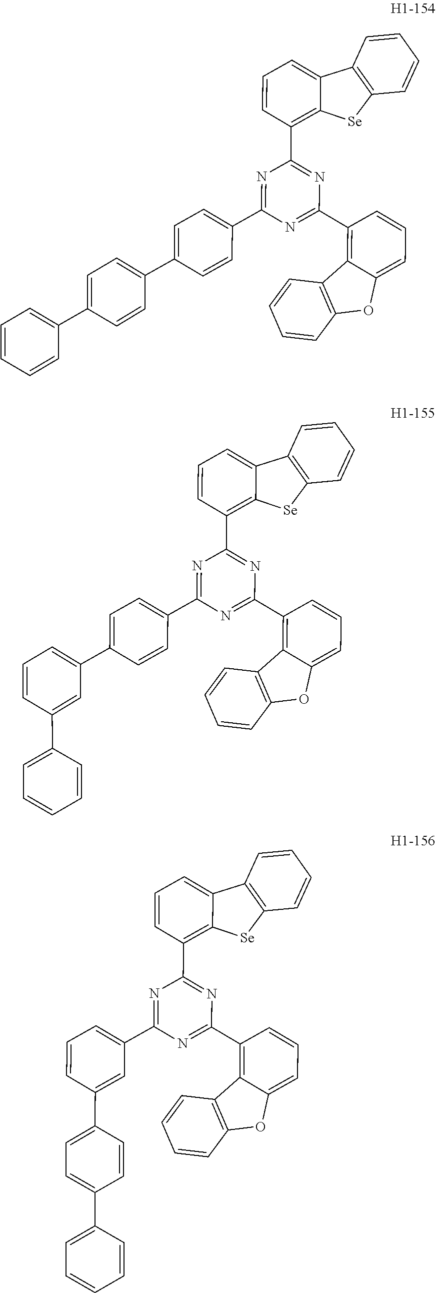 [SMILES: c1ccc(-c2ccc(-c3ccc(-c4nc(-c5cccc6c5[se]c5ccccc56)nc(-c5cccc6oc7ccccc7c56)n4)cc3)cc2)cc1 ; c1ccc(-c2cccc(-c3ccc(-c4nc(-c5cccc6c5[se]c5ccccc56)nc(-c5cccc6oc7ccccc7c56)n4)cc3)c2)cc1 ; c1ccc(-c2ccc(-c3cccc(-c4nc(-c5cccc6c5[se]c5ccccc56)nc(-c5cccc6oc7ccccc7c56)n4)c3)cc2)cc1]

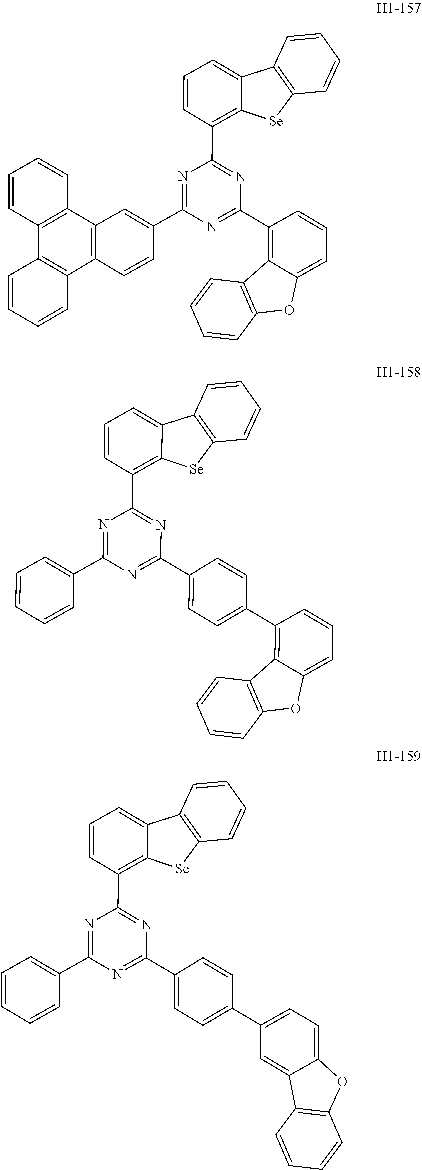

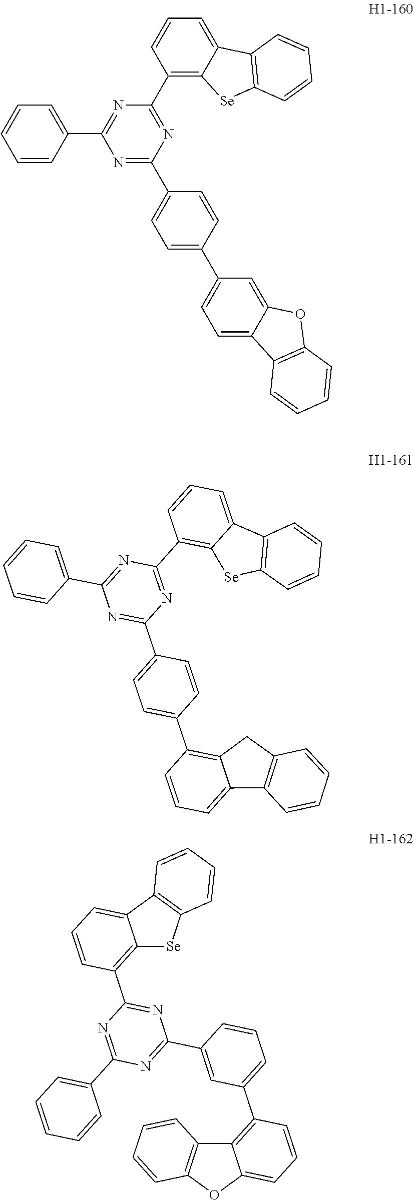

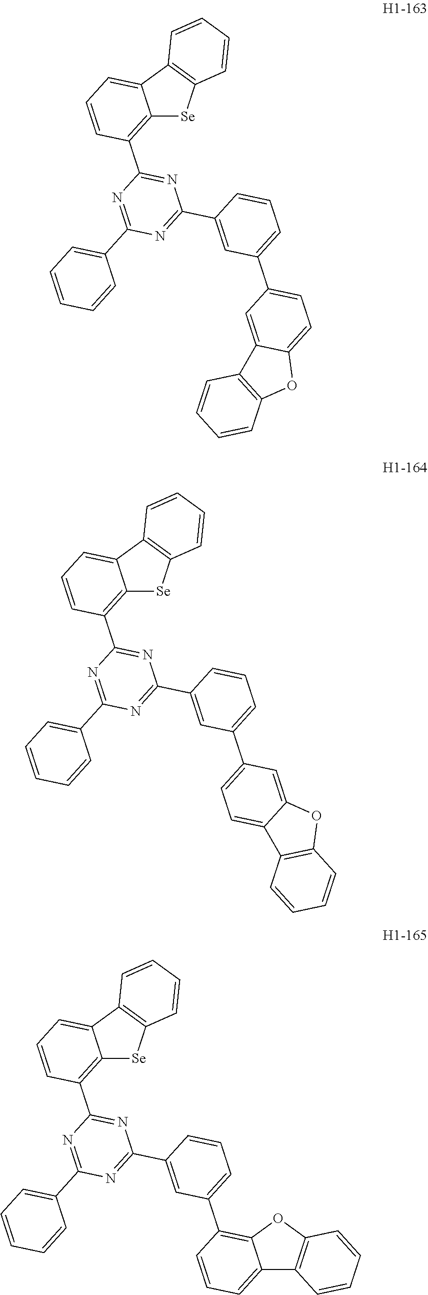 [SMILES: c1ccc(-c2nc(-c3cccc(-c4ccc5oc6ccccc6c5c4)c3)nc(-c3cccc4c3[se]c3ccccc34)n2)cc1 ; c1ccc(-c2nc(-c3cccc(-c4ccc5c(c4)oc4ccccc45)c3)nc(-c3cccc4c3[se]c3ccccc34)n2)cc1 ; c1ccc(-c2nc(-c3cccc(-c4cccc5c4oc4ccccc45)c3)nc(-c3cccc4c3[se]c3ccccc34)n2)cc1]

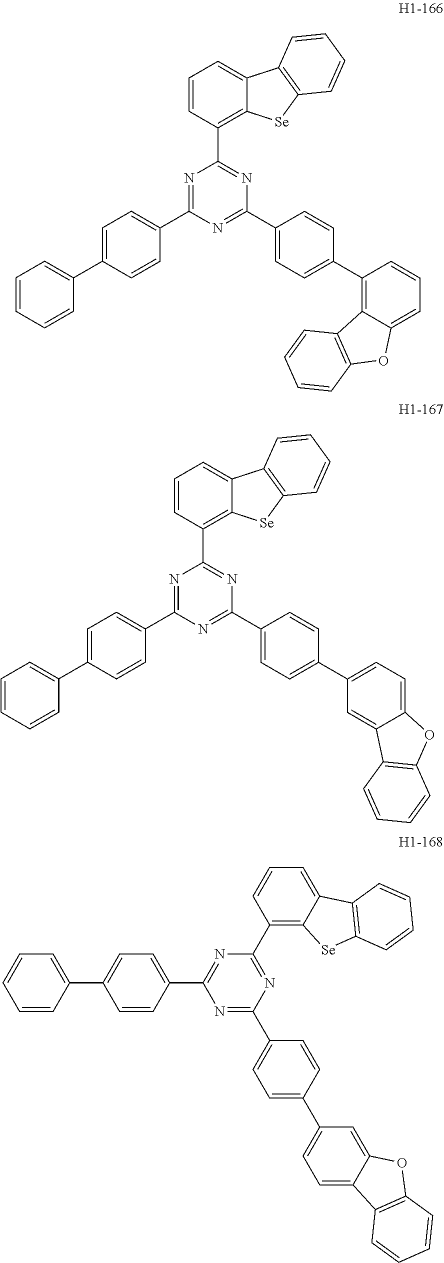 [SMILES: c1ccc(-c2ccc(-c3nc(-c4ccc(-c5cccc6oc7ccccc7c56)cc4)nc(-c4cccc5c4[se]c4ccccc45)n3)cc2)cc1 ; c1ccc(-c2ccc(-c3nc(-c4ccc(-c5ccc6oc7ccccc7c6c5)cc4)nc(-c4cccc5c4[se]c4ccccc45)n3)cc2)cc1 ; c1ccc(-c2ccc(-c3nc(-c4ccc(-c5ccc6c(c5)oc5ccccc56)cc4)nc(-c4cccc5c4[se]c4ccccc45)n3)cc2)cc1]

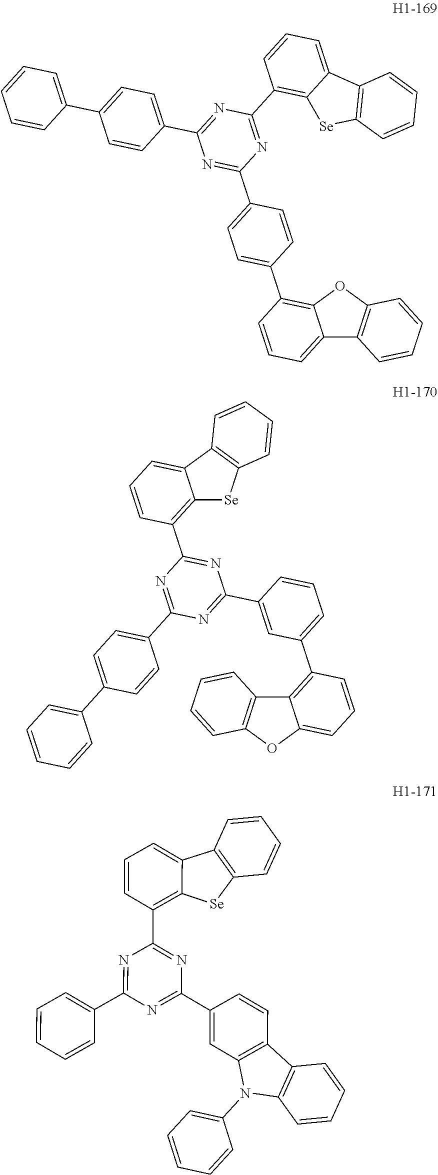 [SMILES: c1ccc(-c2ccc(-c3nc(-c4ccc(-c5cccc6c5oc5ccccc56)cc4)nc(-c4cccc5c4[se]c4ccccc45)n3)cc2)cc1 ; c1ccc(-c2ccc(-c3nc(-c4cccc(-c5cccc6oc7ccccc7c56)c4)nc(-c4cccc5c4[se]c4ccccc45)n3)cc2)cc1 ; c1ccc(-c2nc(-c3ccc4c5ccccc5n(-c5ccccc5)c4c3)nc(-c3cccc4c3[se]c3ccccc34)n2)cc1]

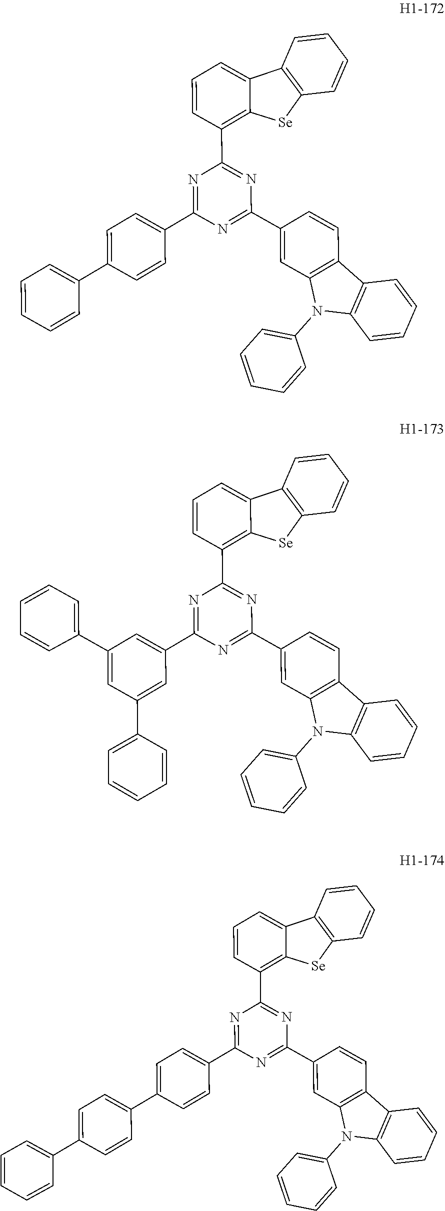 [SMILES: c1ccc(-c2ccc(-c3nc(-c4ccc5c6ccccc6n(-c6ccccc6)c5c4)nc(-c4cccc5c4[se]c4ccccc45)n3)cc2)cc1 ; c1ccc(-c2cc(-c3ccccc3)cc(-c3nc(-c4ccc5c6ccccc6n(-c6ccccc6)c5c4)nc(-c4cccc5c4[se]c4ccccc45)n3)c2)cc1 ; c1ccc(-c2ccc(-c3ccc(-c4nc(-c5ccc6c7ccccc7n(-c7ccccc7)c6c5)nc(-c5cccc6c5[se]c5ccccc56)n4)cc3)cc2)cc1]

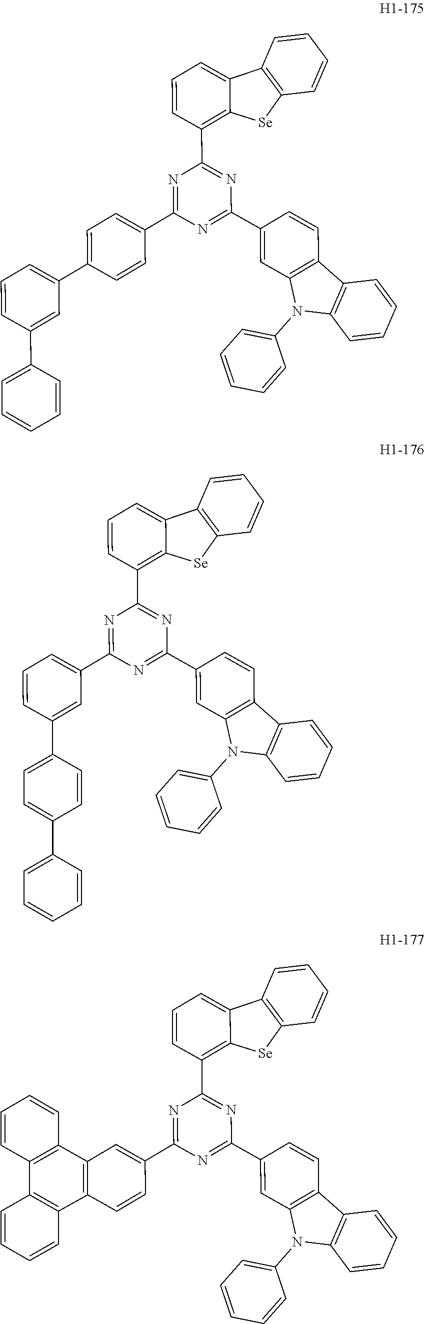 [SMILES: c1ccc(-c2cccc(-c3ccc(-c4nc(-c5ccc6c7ccccc7n(-c7ccccc7)c6c5)nc(-c5cccc6c5[se]c5ccccc56)n4)cc3)c2)cc1 ; c1ccc(-c2ccc(-c3cccc(-c4nc(-c5ccc6c7ccccc7n(-c7ccccc7)c6c5)nc(-c5cccc6c5[se]c5ccccc56)n4)c3)cc2)cc1 ; c1ccc(-n2c3ccccc3c3ccc(-c4nc(-c5ccc6c7ccccc7c7ccccc7c6c5)nc(-c5cccc6c5[se]c5ccccc56)n4)cc32)cc1]

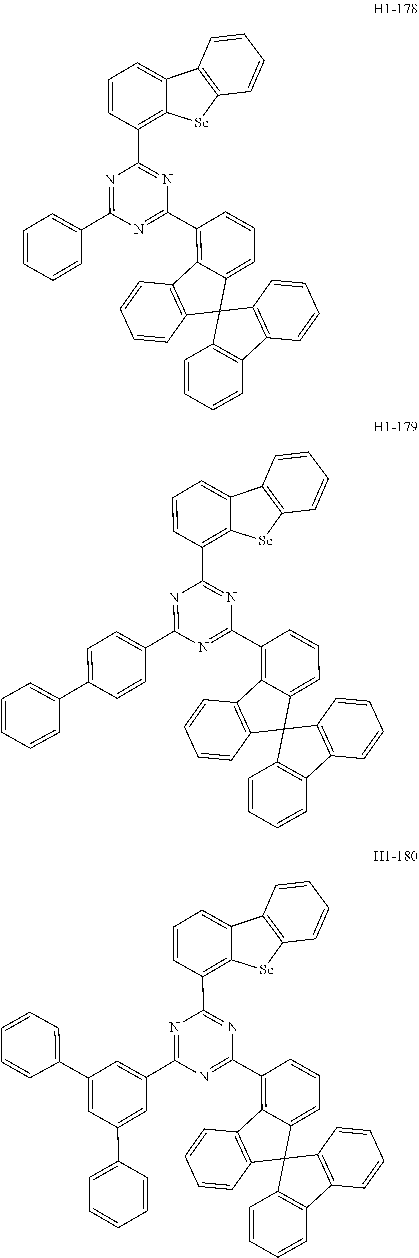 [SMILES: c1ccc(-c2nc(-c3cccc4c3-c3ccccc3C43c4ccccc4-c4ccccc43)nc(-c3cccc4c3[se]c3ccccc34)n2)cc1 ; c1ccc(-c2ccc(-c3nc(-c4cccc5c4-c4ccccc4C54c5ccccc5-c5ccccc54)nc(-c4cccc5c4[se]c4ccccc45)n3)cc2)cc1 ; c1ccc(-c2cc(-c3ccccc3)cc(-c3nc(-c4cccc5c4-c4ccccc4C54c5ccccc5-c5ccccc54)nc(-c4cccc5c4[se]c4ccccc45)n3)c2)cc1]

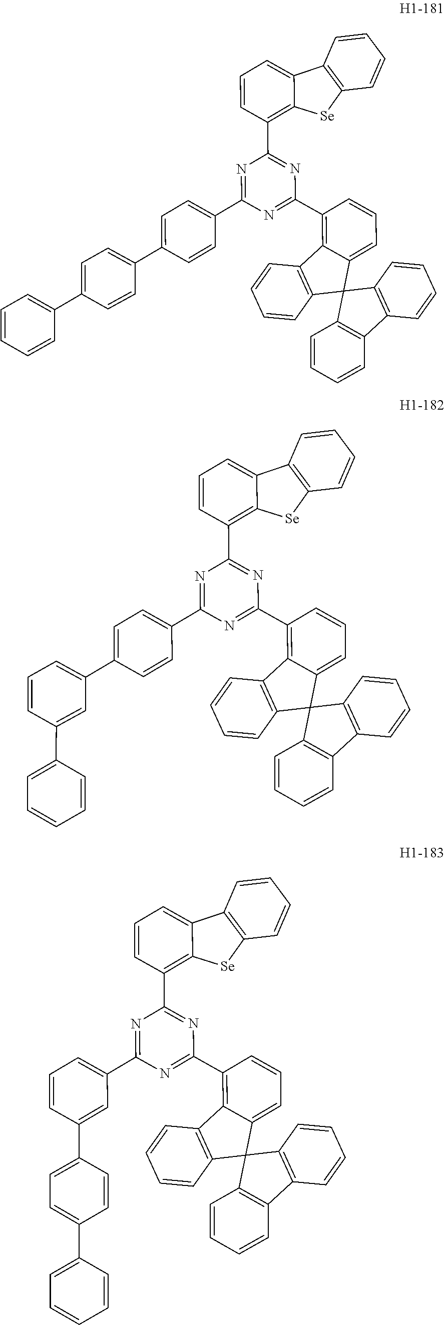 [SMILES: c1ccc(-c2ccc(-c3ccc(-c4nc(-c5cccc6c5-c5ccccc5C65c6ccccc6-c6ccccc65)nc(-c5cccc6c5[se]c5ccccc56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6c5[se]c5ccccc56)n4)cc3)c2)cc1 ; c1ccc(-c2ccc(-c3cccc(-c4nc(-c5cccc6c5-c5ccccc5C65c6ccccc6-c6ccccc65)nc(-c5cccc6c5[se]c5ccccc56)n4)c3)cc2)cc1]

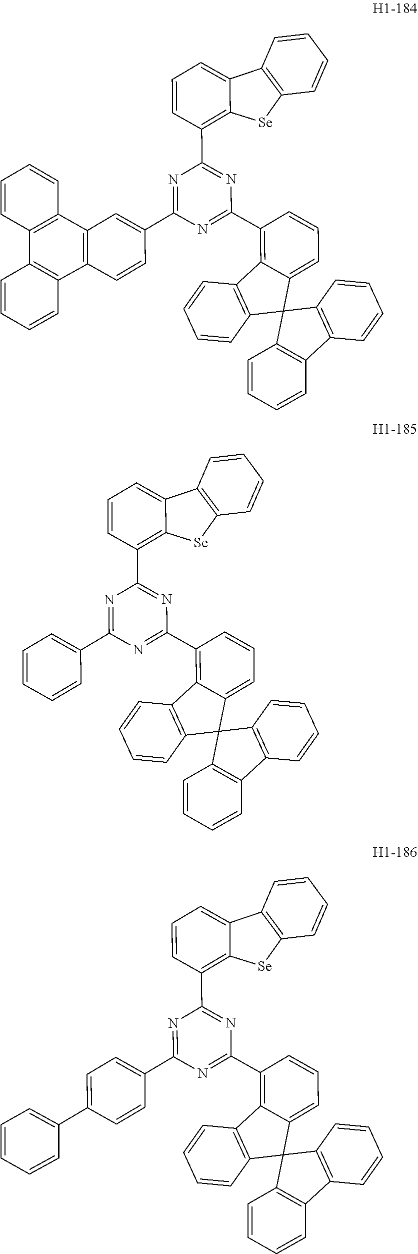 [SMILES: c1ccc2c(c1)-c1ccccc1C21c2ccccc2-c2c(-c3nc(-c4ccc5c6ccccc6c6ccccc6c5c4)nc(-c4cccc5c4[se]c4ccccc45)n3)cccc21 ; c1ccc(-c2nc(-c3cccc4c3-c3ccccc3C43c4ccccc4-c4ccccc43)nc(-c3cccc4c3[se]c3ccccc34)n2)cc1 ; c1ccc(-c2ccc(-c3nc(-c4cccc5c4-c4ccccc4C54c5ccccc5-c5ccccc54)nc(-c4cccc5c4[se]c4ccccc45)n3)cc2)cc1]

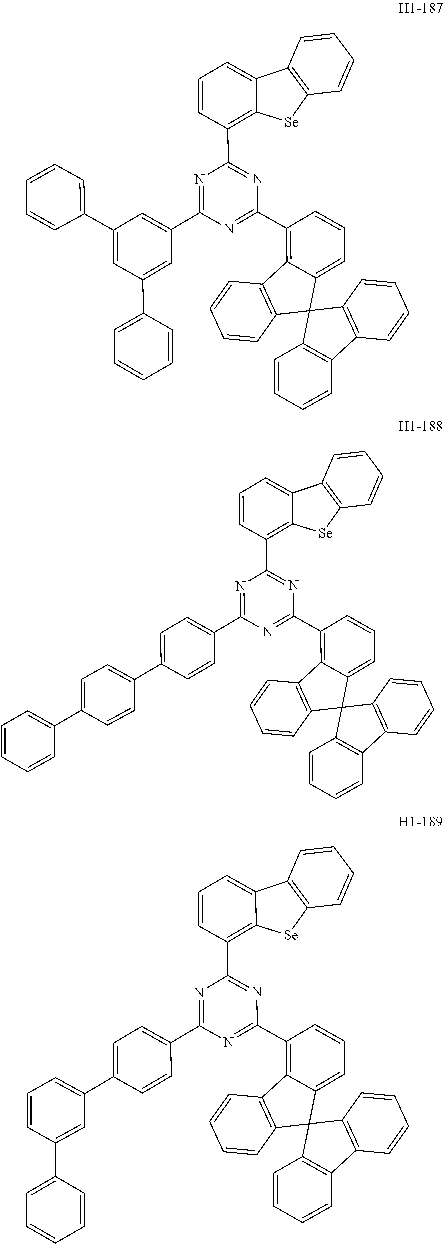 [SMILES: c1ccc(-c2cc(-c3ccccc3)cc(-c3nc(-c4cccc5c4-c4ccccc4C54c5ccccc5-c5ccccc54)nc(-c4cccc5c4[se]c4ccccc45)n3)c2)cc1 ; c1ccc(-c2ccc(-c3ccc(-c4nc(-c5cccc6c5-c5ccccc5C65c6ccccc6-c6ccccc65)nc(-c5cccc6c5[se]c5ccccc56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6c5[se]c5ccccc56)n4)cc3)c2)cc1]

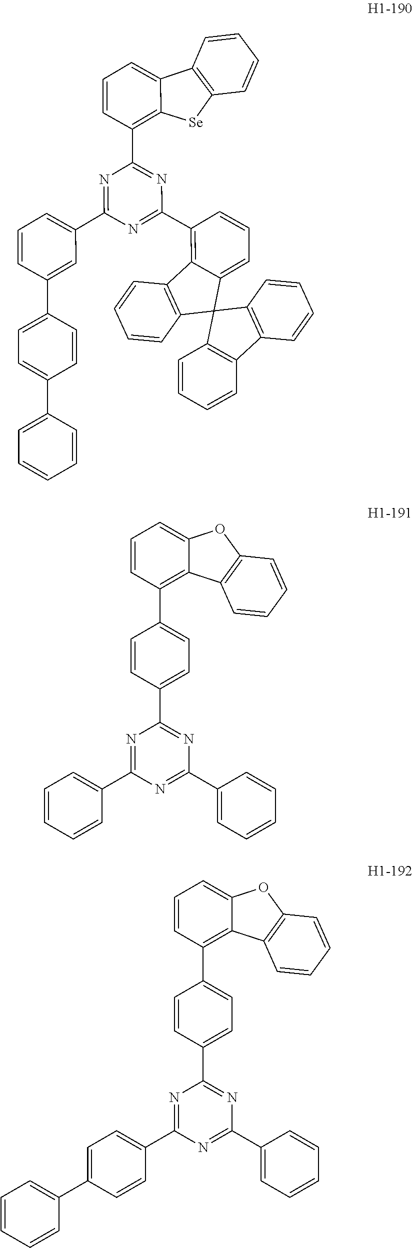 [SMILES: c1ccc(-c2ccc(-c3cccc(-c4nc(-c5cccc6c5-c5ccccc5C65c6ccccc6-c6ccccc65)nc(-c5cccc6c5[se]c5ccccc56)n4)c3)cc2)cc1 ; c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1]

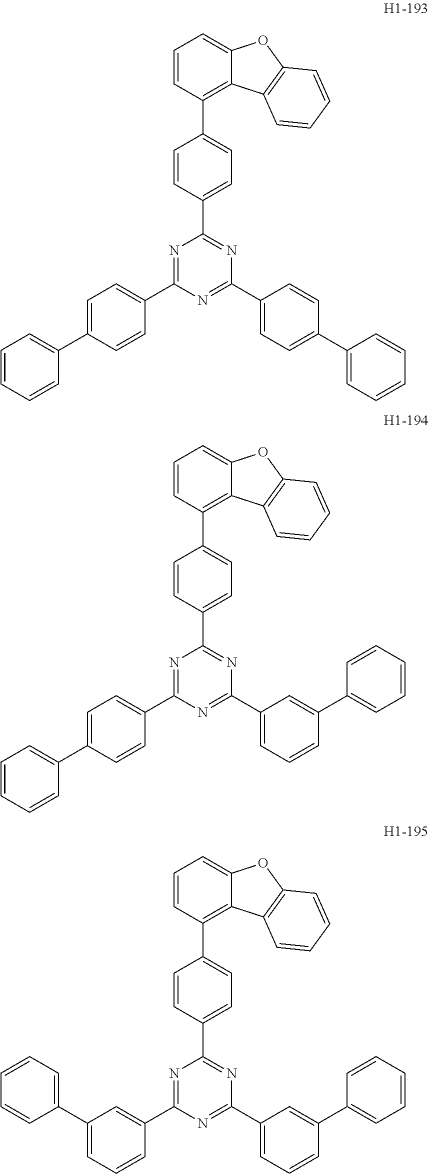 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1]

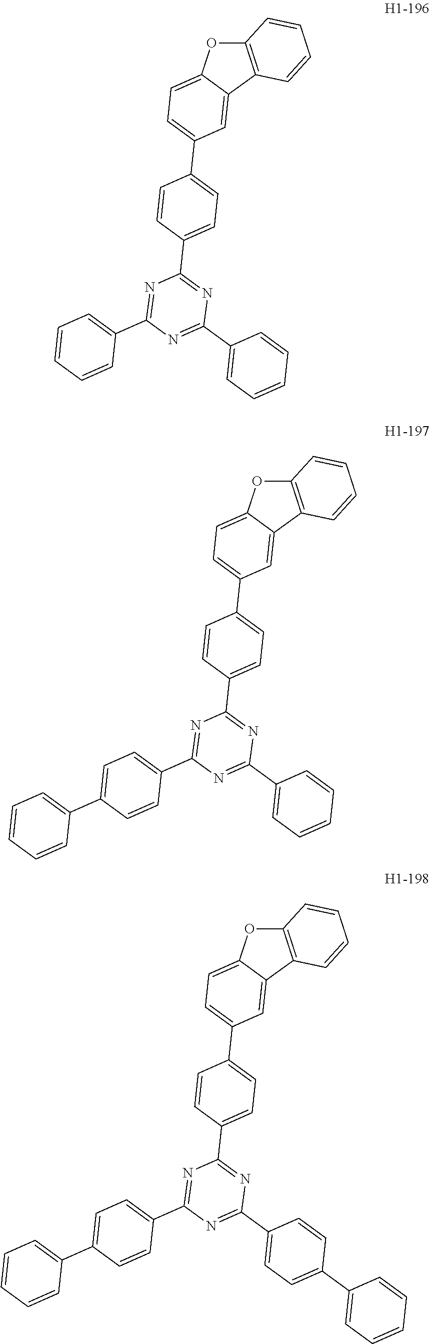 [SMILES: c1ccc(-c2nc(-c3ccccc3)nc(-c3ccc(-c4ccc5oc6ccccc6c5c4)cc3)n2)cc1 ; c1ccc(-c2ccc(-c3nc(-c4ccccc4)nc(-c4ccc(-c5ccc6oc7ccccc7c6c5)cc4)n3)cc2)cc1 ; c1ccc(-c2ccc(-c3nc(-c4ccc(-c5ccccc5)cc4)nc(-c4ccc(-c5ccc6oc7ccccc7c6c5)cc4)n3)cc2)cc1]

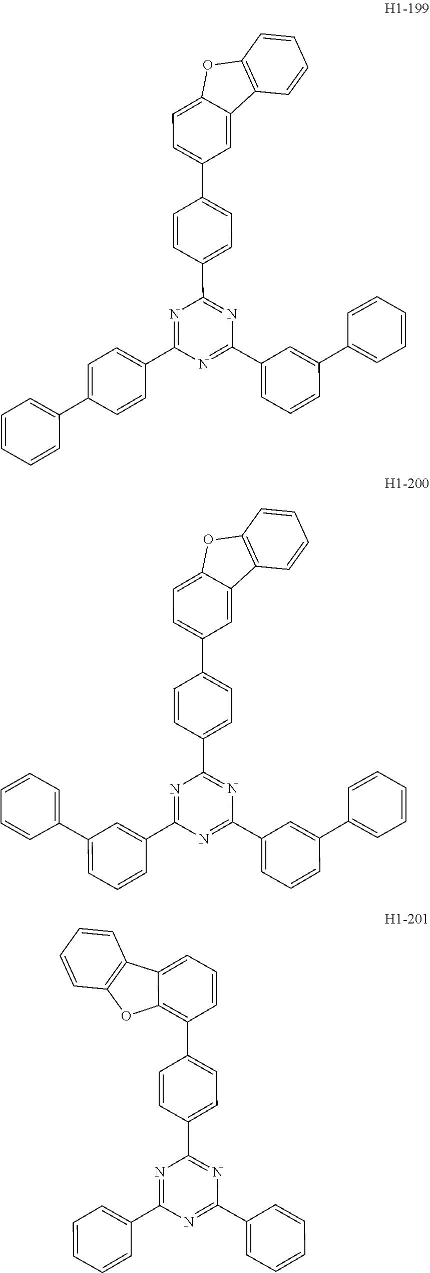

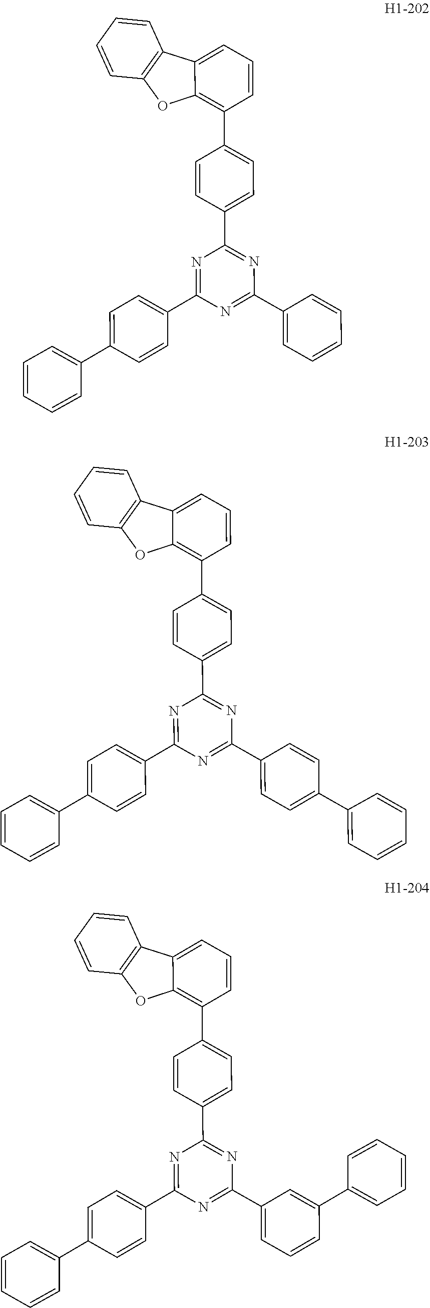 [SMILES: c1ccc(-c2ccc(-c3nc(-c4ccccc4)nc(-c4ccc(-c5cccc6c5oc5ccccc56)cc4)n3)cc2)cc1 ; c1ccc(-c2ccc(-c3nc(-c4ccc(-c5ccccc5)cc4)nc(-c4ccc(-c5cccc6c5oc5ccccc56)cc4)n3)cc2)cc1 ; c1ccc(-c2ccc(-c3nc(-c4ccc(-c5cccc6c5oc5ccccc56)cc4)nc(-c4cccc(-c5ccccc5)c4)n3)cc2)cc1]

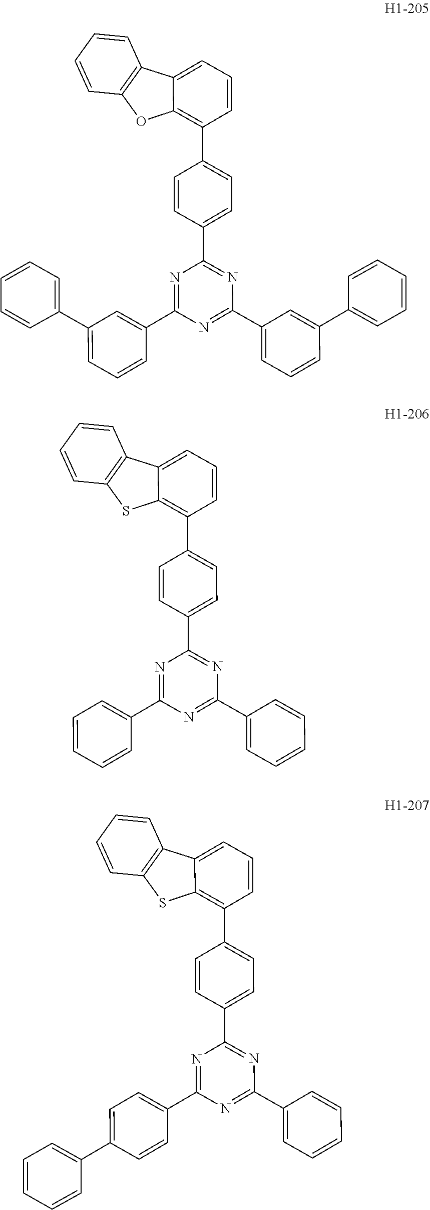 [SMILES: c1ccc(-c2cccc(-c3nc(-c4ccc(-c5cccc6c5oc5ccccc56)cc4)nc(-c4cccc(-c5ccccc5)c4)n3)c2)cc1 ; c1ccc(-c2nc(-c3ccccc3)nc(-c3ccc(-c4cccc5c4sc4ccccc45)cc3)n2)cc1 ; c1ccc(-c2ccc(-c3nc(-c4ccccc4)nc(-c4ccc(-c5cccc6c5sc5ccccc56)cc4)n3)cc2)cc1]

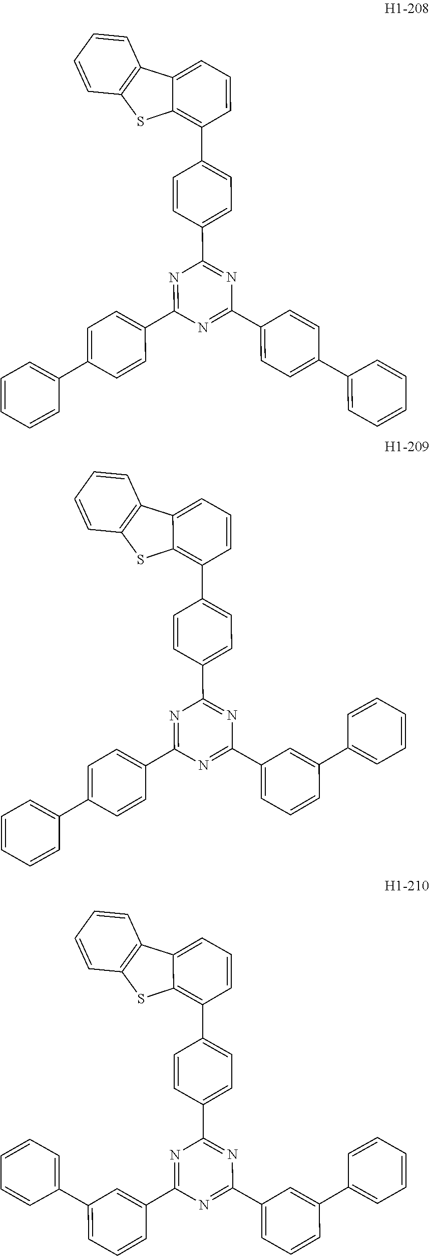 [SMILES: c1ccc(-c2ccc(-c3nc(-c4ccc(-c5ccccc5)cc4)nc(-c4ccc(-c5cccc6c5sc5ccccc56)cc4)n3)cc2)cc1 ; c1ccc(-c2ccc(-c3nc(-c4ccc(-c5cccc6c5sc5ccccc56)cc4)nc(-c4cccc(-c5ccccc5)c4)n3)cc2)cc1 ; c1ccc(-c2cccc(-c3nc(-c4ccc(-c5cccc6c5sc5ccccc56)cc4)nc(-c4cccc(-c5ccccc5)c4)n3)c2)cc1]

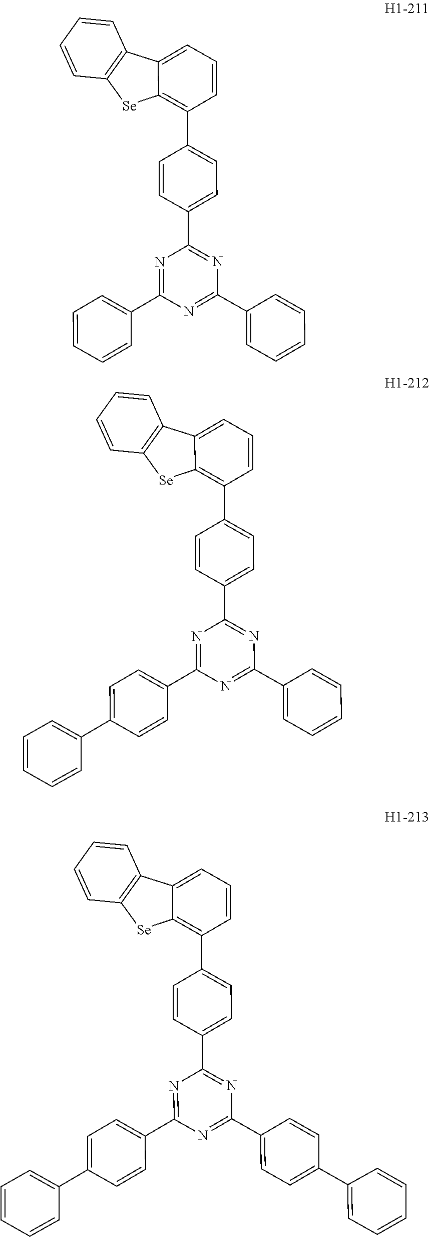

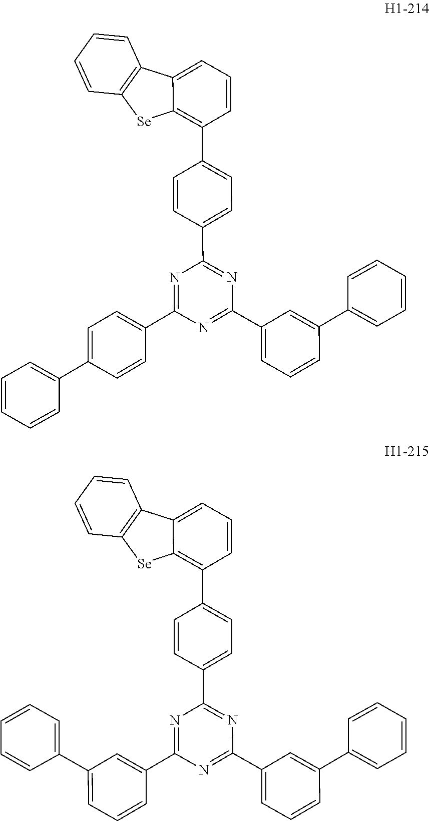 [SMILES: c1ccc(-c2ccc(-c3nc(-c4ccc(-c5cccc6c5[se]c5ccccc56)cc4)nc(-c4cccc(-c5ccccc5)c4)n3)cc2)cc1 ; c1ccc(-c2cccc(-c3nc(-c4ccc(-c5cccc6c5[se]c5ccccc56)cc4)nc(-c4cccc(-c5ccccc5)c4)n3)c2)cc1]

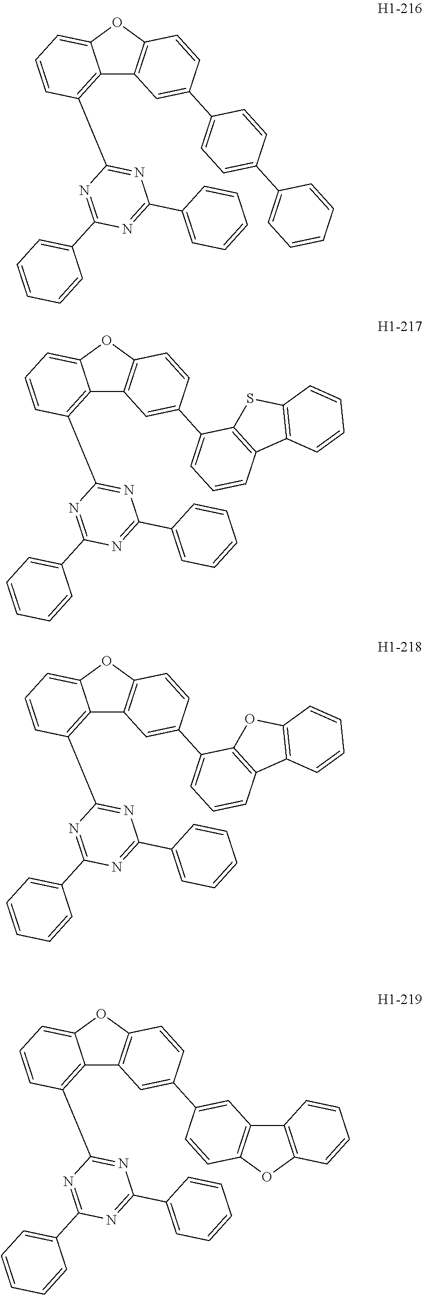 [SMILES: c1ccc(-c2ccc(-c3ccc4oc5cccc(-c6nc(-c7ccccc7)nc(-c7ccccc7)n6)c5c4c3)cc2)cc1 ; c1ccc(-c2nc(-c3ccccc3)nc(-c3cccc4oc5ccc(-c6cccc7c6sc6ccccc67)cc5c34)n2)cc1 ; c1ccc(-c2nc(-c3ccccc3)nc(-c3cccc4oc5ccc(-c6cccc7c6oc6ccccc67)cc5c34)n2)cc1 ; c1ccc(-c2nc(-c3ccccc3)nc(-c3cccc4oc5ccc(-c6ccc7oc8ccccc8c7c6)cc5c34)n2)cc1]

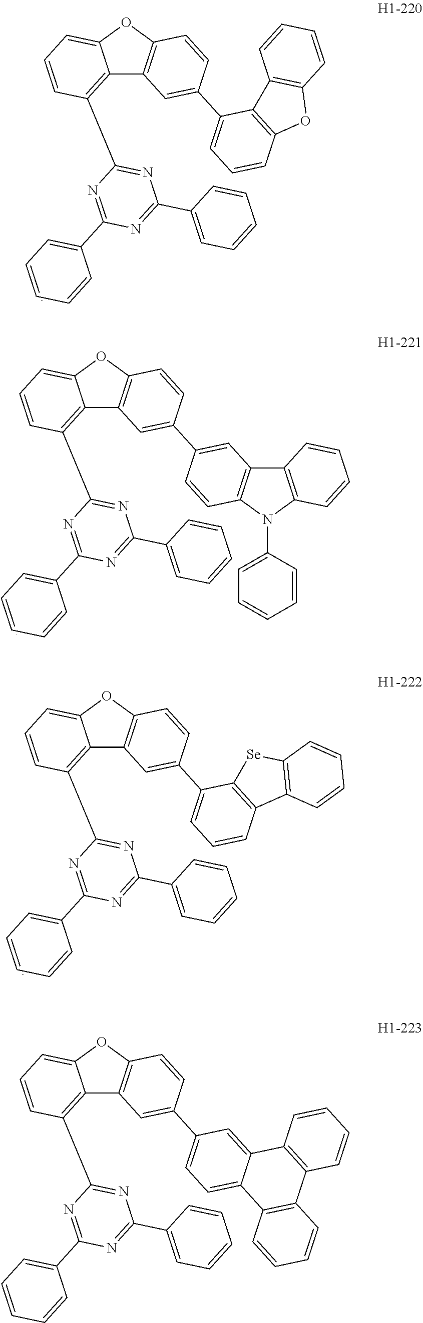

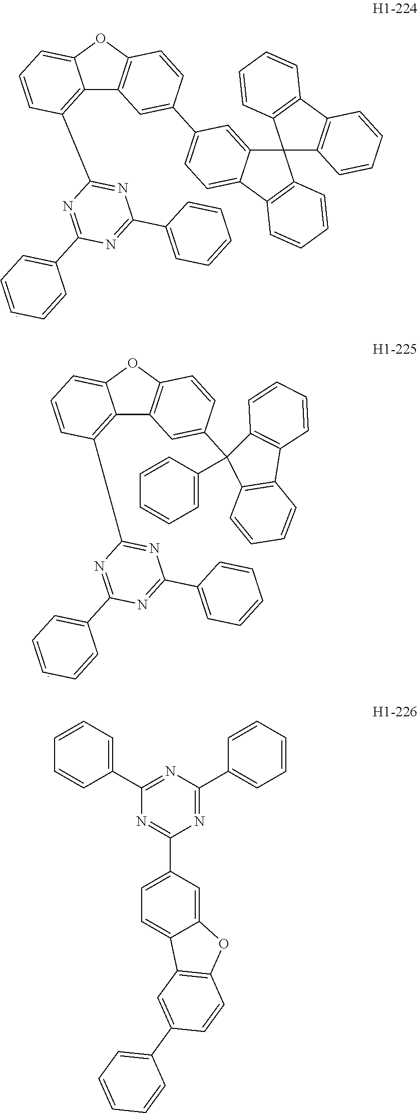

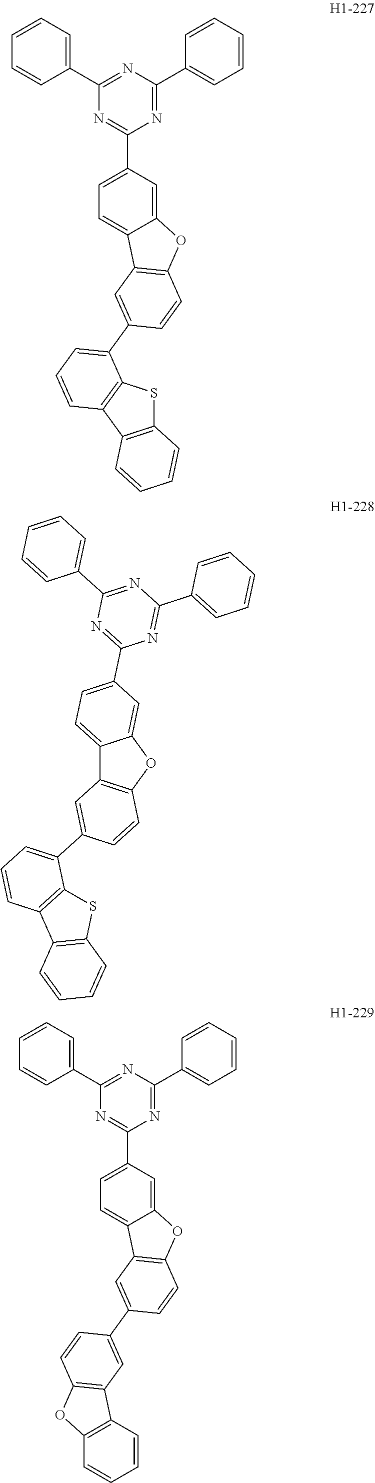

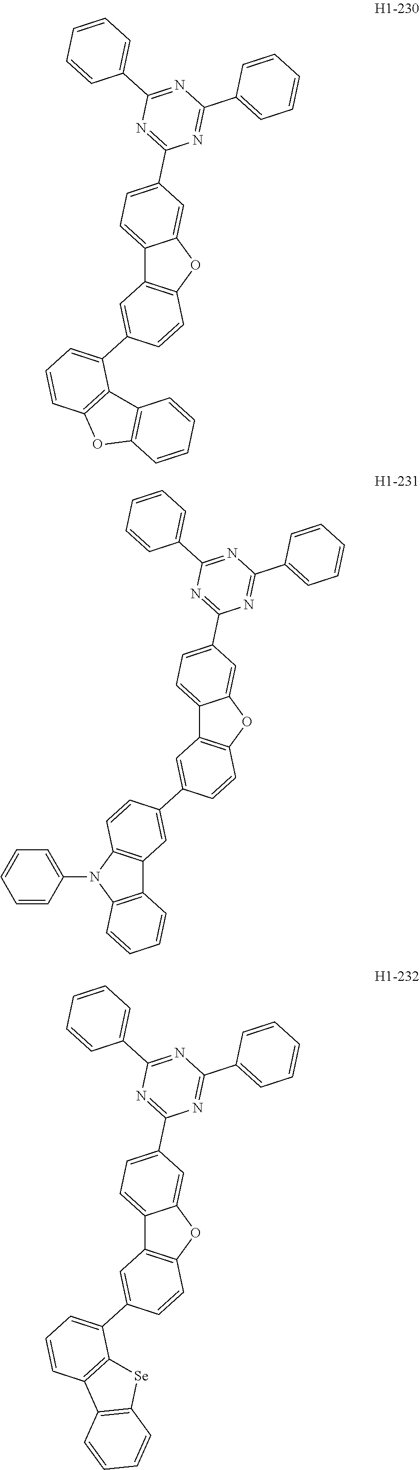

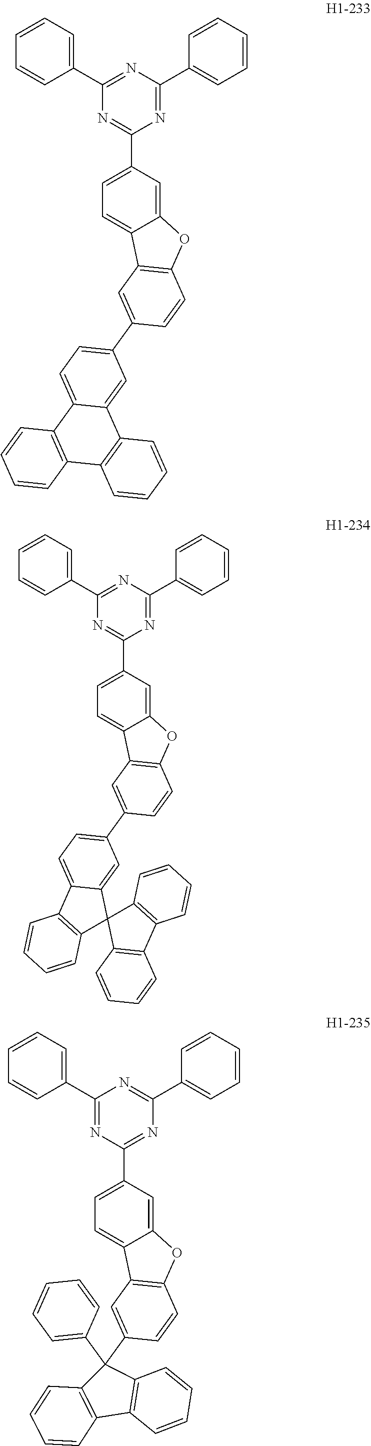

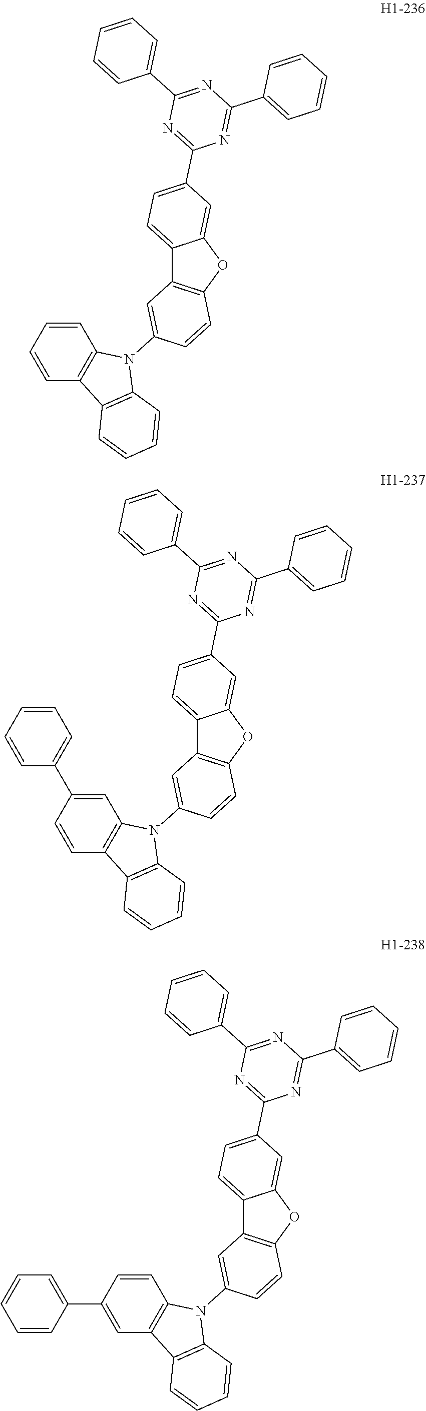

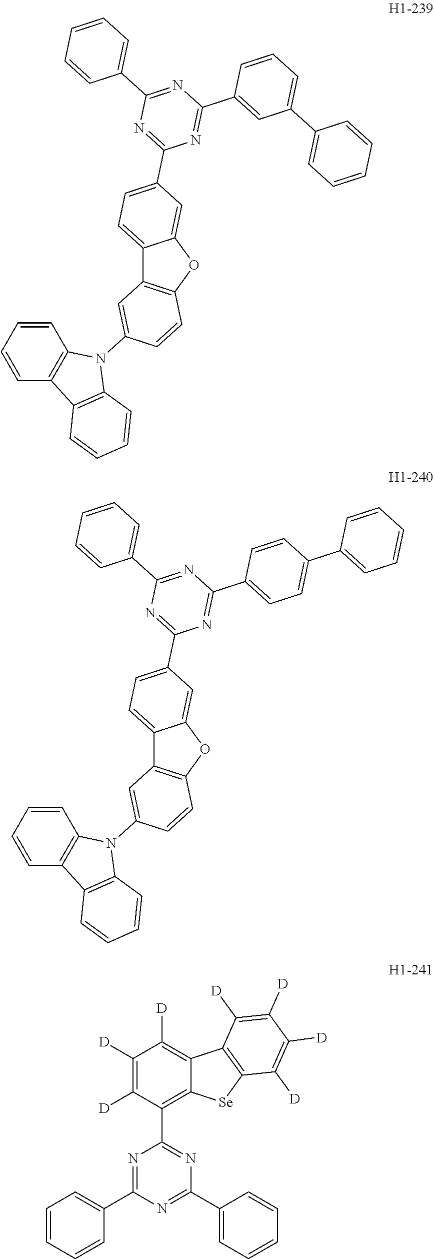 [SMILES: c1ccc(-c2cccc(-c3nc(-c4ccccc4)nc(-c4ccc5c(c4)oc4ccc(-n6c7ccccc7c7ccccc76)cc45)n3)c2)cc1 ; c1ccc(-c2ccc(-c3nc(-c4ccccc4)nc(-c4ccc5c(c4)oc4ccc(-n6c7ccccc7c7ccccc76)cc45)n3)cc2)cc1 ; [2H]c1c([2H])c([2H])c2c([se]c3c(-c4nc(-c5ccccc5)nc(-c5ccccc5)n4)c([2H])c([2H])c([2H])c32)c1[2H]]

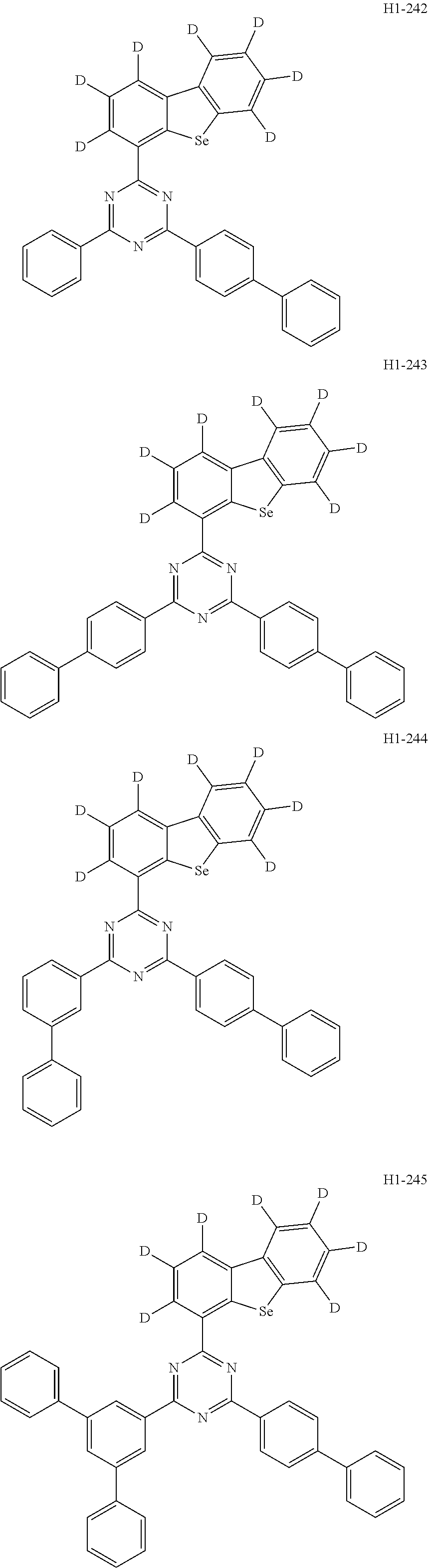 [SMILES: [2H]c1c([2H])c([2H])c2c([se]c3c(-c4nc(-c5ccccc5)nc(-c5ccc(-c6ccccc6)cc5)n4)c([2H])c([2H])c([2H])c32)c1[2H] ; [2H]c1c([2H])c([2H])c2c([se]c3c(-c4nc(-c5ccc(-c6ccccc6)cc5)nc(-c5ccc(-c6ccccc6)cc5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6ccccc6)c5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cc(-c6ccccc6)cc(-c6ccccc6)c5)n4)c([2H])c([2H])c([2H])c32)c1[2H]]

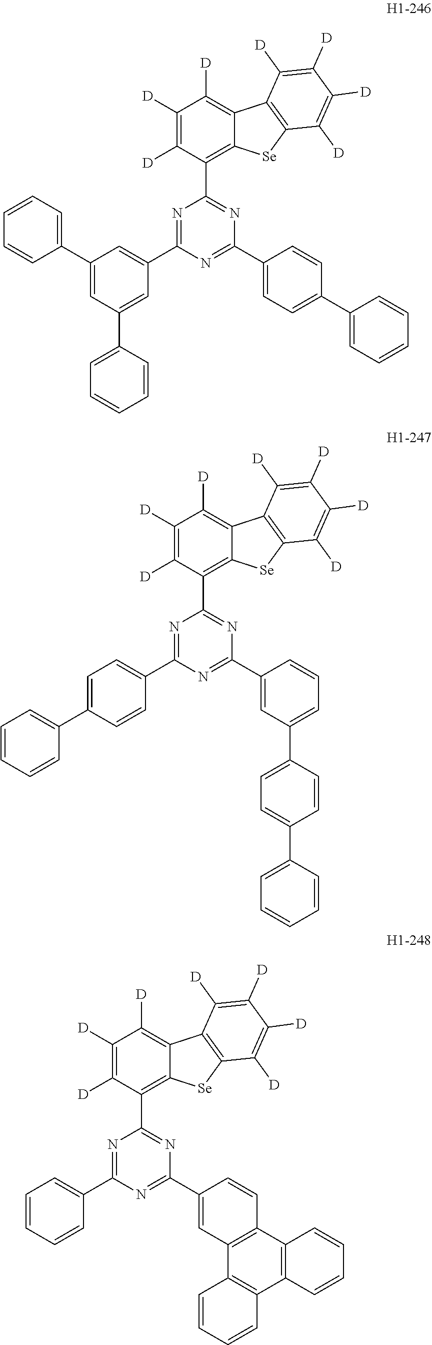 [SMILES: [2H]c1c([2H])c([2H])c2c([se]c3c(-c4nc(-c5ccc(-c6ccccc6)cc5)nc(-c5cc(-c6ccccc6)cc(-c6ccccc6)c5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6ccc(-c7ccccc7)cc6)c5)n4)c([2H])c([2H])c([2H])c32)c1[2H] ; [2H]c1c([2H])c([2H])c2c([se]c3c(-c4nc(-c5ccccc5)nc(-c5ccc6c7ccccc7c7ccccc7c6c5)n4)c([2H])c([2H])c([2H])c32)c1[2H]]

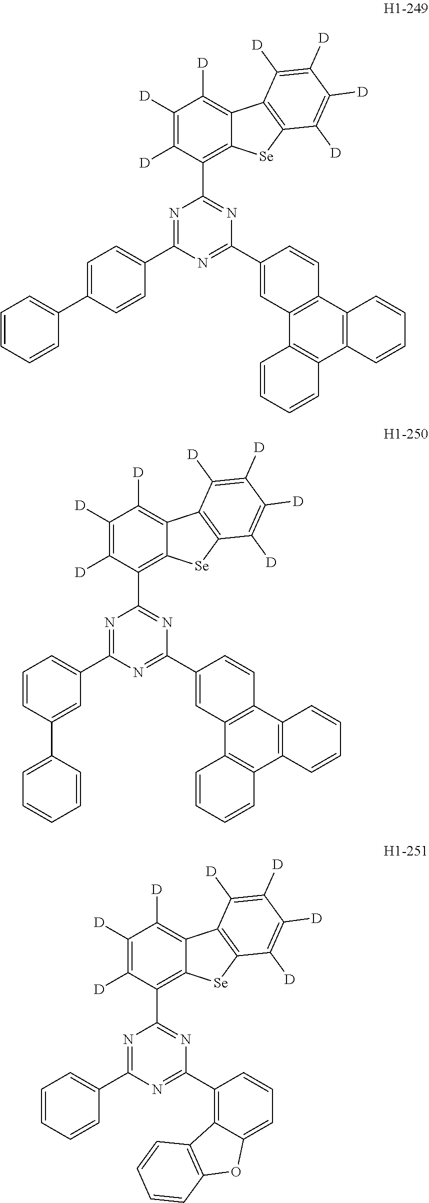

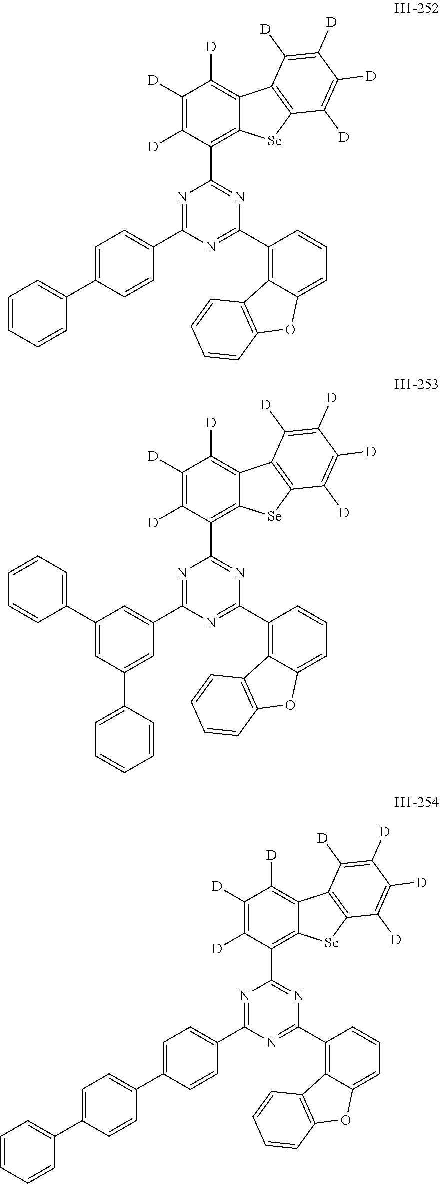 [SMILES: [2H]c1c([2H])c([2H])c2c([se]c3c(-c4nc(-c5ccc(-c6ccccc6)cc5)nc(-c5cccc6oc7ccccc7c56)n4)c([2H])c([2H])c([2H])c32)c1[2H] ; [2H]c1c([2H])c([2H])c2c([se]c3c(-c4nc(-c5cc(-c6ccccc6)cc(-c6ccccc6)c5)nc(-c5cccc6oc7ccccc7c56)n4)c([2H])c([2H])c([2H])c32)c1[2H] ; [2H]c1c([2H])c([2H])c2c([se]c3c(-c4nc(-c5ccc(-c6ccc(-c7ccccc7)cc6)cc5)nc(-c5cccc6oc7ccccc7c56)n4)c([2H])c([2H])c([2H])c32)c1[2H]]

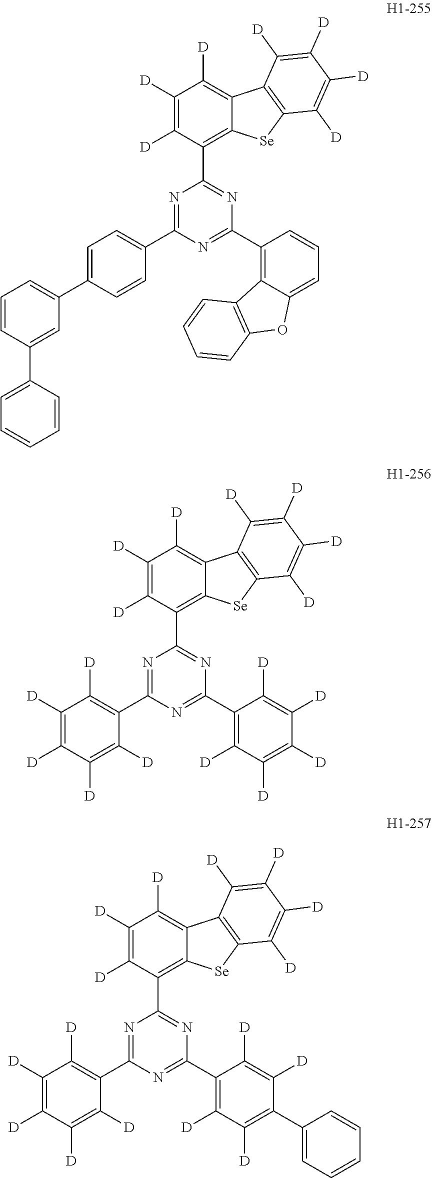 [SMILES: [2H]c1c([2H])c([2H])c2c([se]c3c(-c4nc(-c5ccc(-c6cccc(-c7ccccc7)c6)cc5)nc(-c5cccc6oc7ccccc7c56)n4)c([2H])c([2H])c([2H])c32)c1[2H] ; [2H]c1c([2H])c([2H])c(-c2nc(-c3c([2H])c([2H])c([2H])c([2H])c3[2H])nc(-c3c([2H])c([2H])c([2H])c4c3[se]c3c([2H])c([2H])c([2H])c([2H])c34)n2)c([2H])c1[2H] ; [2H]c1c([2H])c([2H])c(-c2nc(-c3c([2H])c([2H])c(-c4ccccc4)c([2H])c3[2H])nc(-c3c([2H])c([2H])c([2H])c4c3[se]c3c([2H])c([2H])c([2H])c([2H])c34)n2)c([2H])c1[2H]]

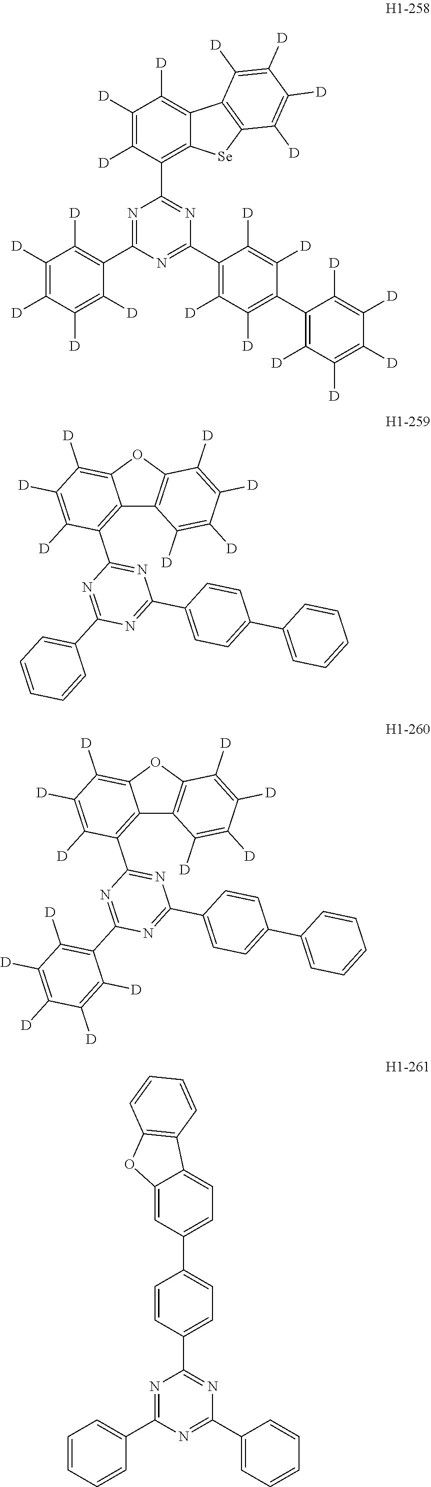 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4c3[se]c3c([2H])c([2H])c([2H])c([2H])c34)n2)c([2H])c1[2H] ; [2H]c1c([2H])c([2H])c2c(oc3c([2H])c([2H])c([2H])c(-c4nc(-c5ccccc5)nc(-c5ccc(-c6ccccc6)cc5)n4)c32)c1[2H] ; [2H]c1c([2H])c([2H])c(-c2nc(-c3ccc(-c4ccccc4)cc3)nc(-c3c([2H])c([2H])c([2H])c4oc5c([2H])c([2H])c([2H])c([2H])c5c34)n2)c([2H])c1[2H] ; c1ccc(-c2nc(-c3ccccc3)nc(-c3ccc(-c4ccc5c(c4)oc4ccccc45)cc3)n2)cc1]

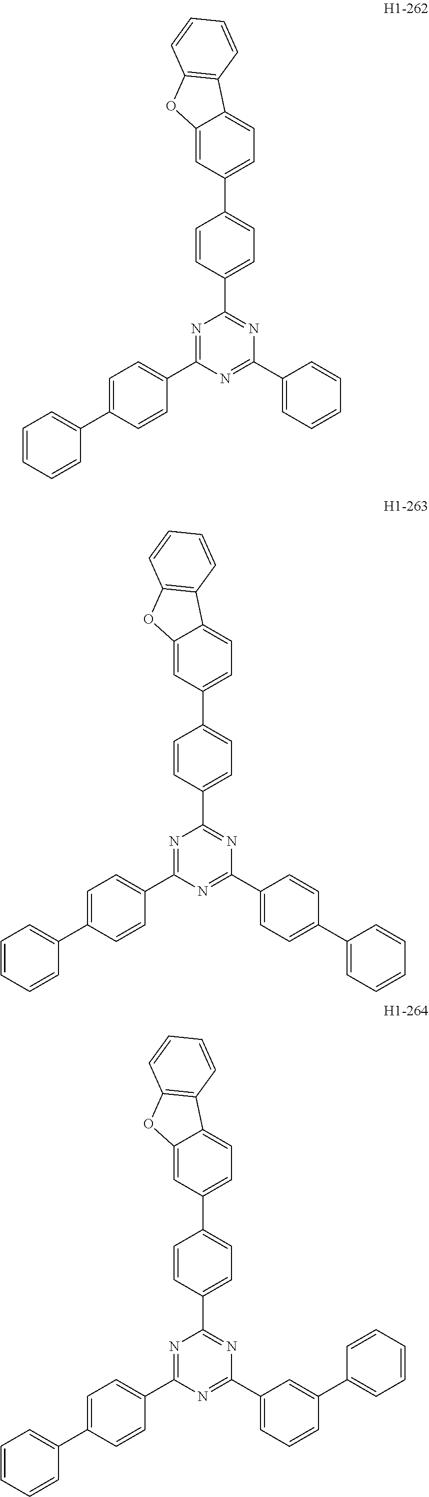 [SMILES: c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3nc(-c4ccc(-c5ccccc5)cc4)nc(-c4ccc(-c5ccc6c(c5)oc5ccccc56)cc4)n3)cc2)cc1 ; c1ccc(-c2ccc(-c3nc(-c4ccc(-c5ccc6c(c5)oc5ccccc56)cc4)nc(-c4cccc(-c5ccccc5)c4)n3)cc2)cc1]

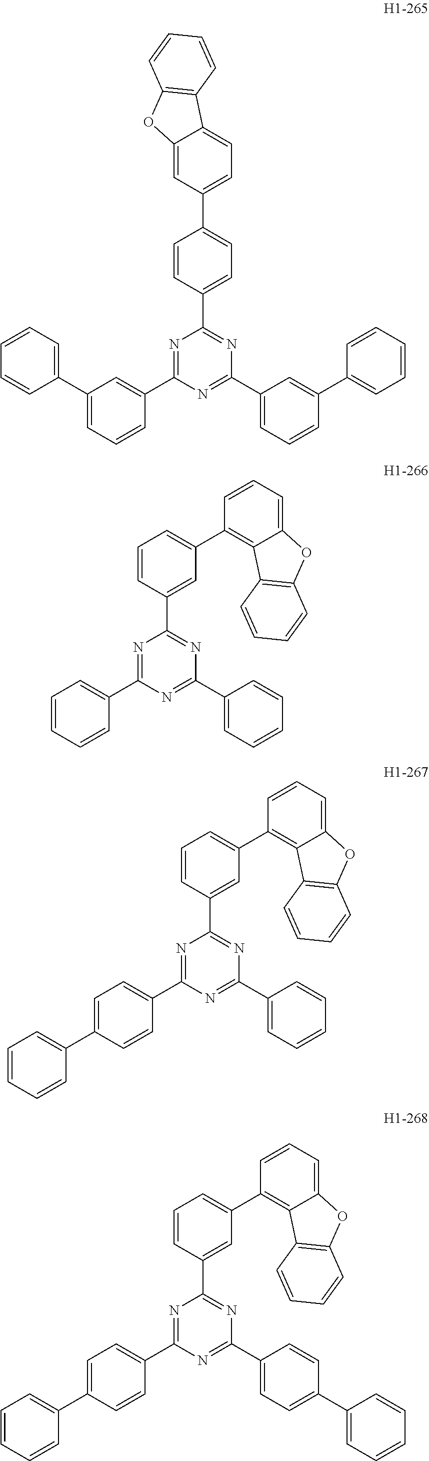 [SMILES: c1ccc(-c2cccc(-c3nc(-c4ccc(-c5ccc6c(c5)oc5ccccc56)cc4)nc(-c4cccc(-c5ccccc5)c4)n3)c2)cc1 ; c1ccc(-c2nc(-c3ccccc3)nc(-c3cccc(-c4cccc5oc6ccccc6c45)c3)n2)cc1 ; c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1]

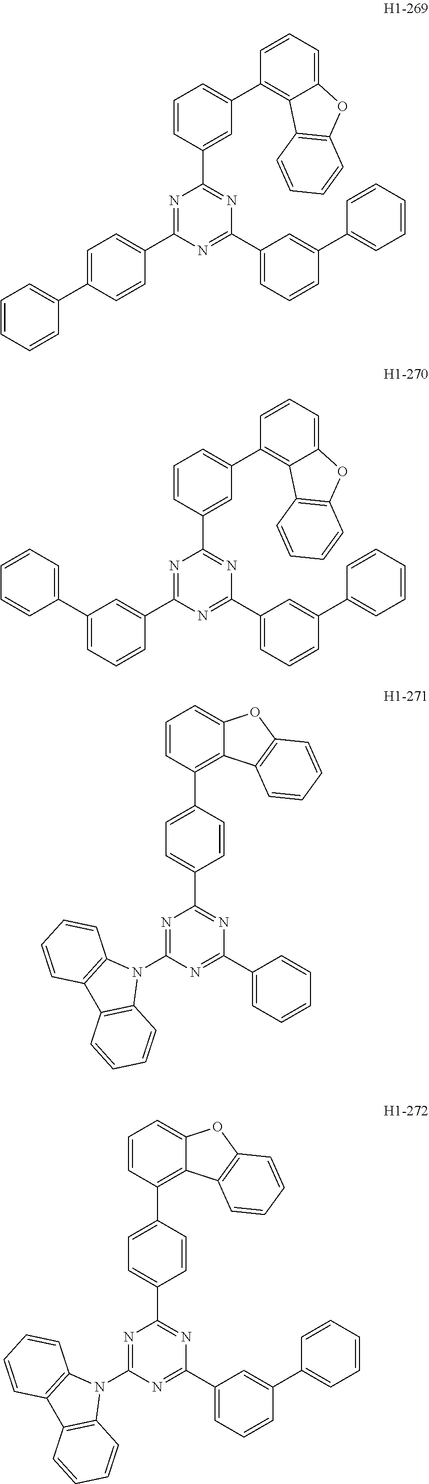 [SMILES: c1ccc(-c2ccc(-c3nc(-c4cccc(-c5ccccc5)c4)nc(-c4cccc(-c5cccc6oc7ccccc7c56)c4)n3)cc2)cc1 ; c1ccc(-c2cccc(-c3nc(-c4cccc(-c5ccccc5)c4)nc(-c4cccc(-c5cccc6oc7ccccc7c56)c4)n3)c2)cc1 ; c1ccc(-c2nc(-c3ccc(-c4cccc5oc6ccccc6c45)cc3)nc(-n3c4ccccc4c4ccccc43)n2)cc1 ; c1ccc(-c2cccc(-c3nc(-c4ccc(-c5cccc6oc7ccccc7c56)cc4)nc(-n4c5ccccc5c5ccccc54)n3)c2)cc1]

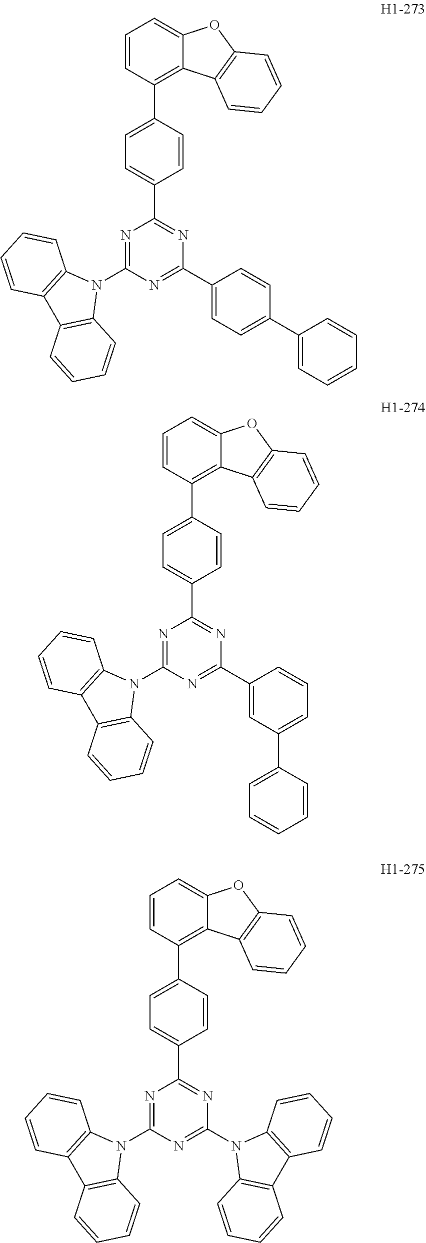 [SMILES: c1ccc(-c2ccc(-c3nc(-c4ccc(-c5cccc6oc7ccccc7c56)cc4)nc(-n4c5ccccc5c5ccccc54)n3)cc2)cc1 ; c1ccc(-c2cccc(-c3nc(-c4ccc(-c5cccc6oc7ccccc7c56)cc4)nc(-n4c5ccccc5c5ccccc54)n3)c2)cc1 ; c1ccc2c(c1)oc1cccc(-c3ccc(-c4nc(-n5c6ccccc6c6ccccc65)nc(-n5c6ccccc6c6ccccc65)n4)cc3)c12]

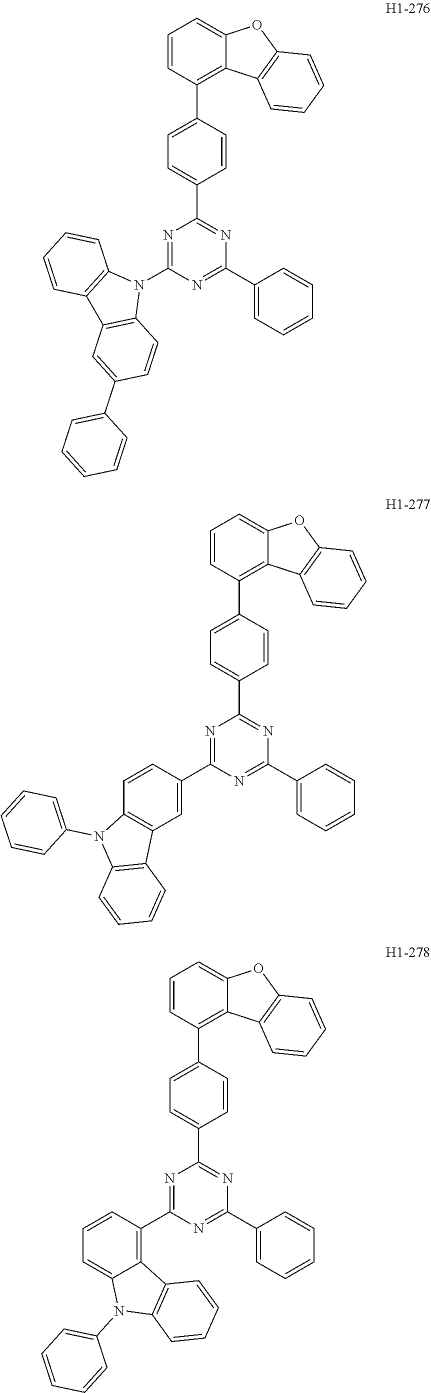 [SMILES: c1ccc(-c2ccc3c(c2)c2ccccc2n3-c2nc(-c3ccccc3)nc(-c3ccc(-c4cccc5oc6ccccc6c45)cc3)n2)cc1 ; c1ccc(-c2nc(-c3ccc(-c4cccc5oc6ccccc6c45)cc3)nc(-c3ccc4c(c3)c3ccccc3n4-c3ccccc3)n2)cc1 ; c1ccc(-c2nc(-c3ccc(-c4cccc5oc6ccccc6c45)cc3)nc(-c3cccc4c3c3ccccc3n4-c3ccccc3)n2)cc1]

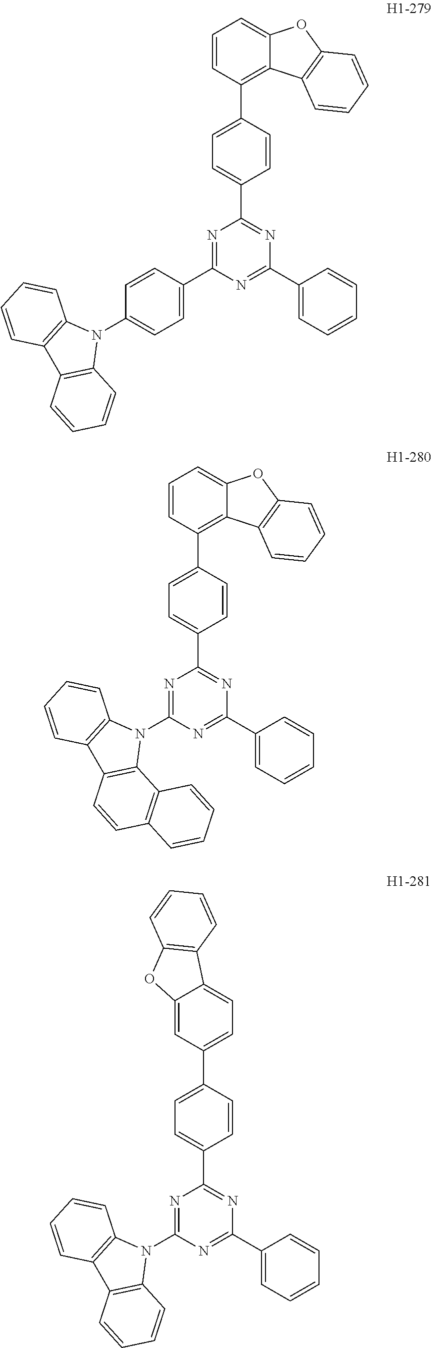

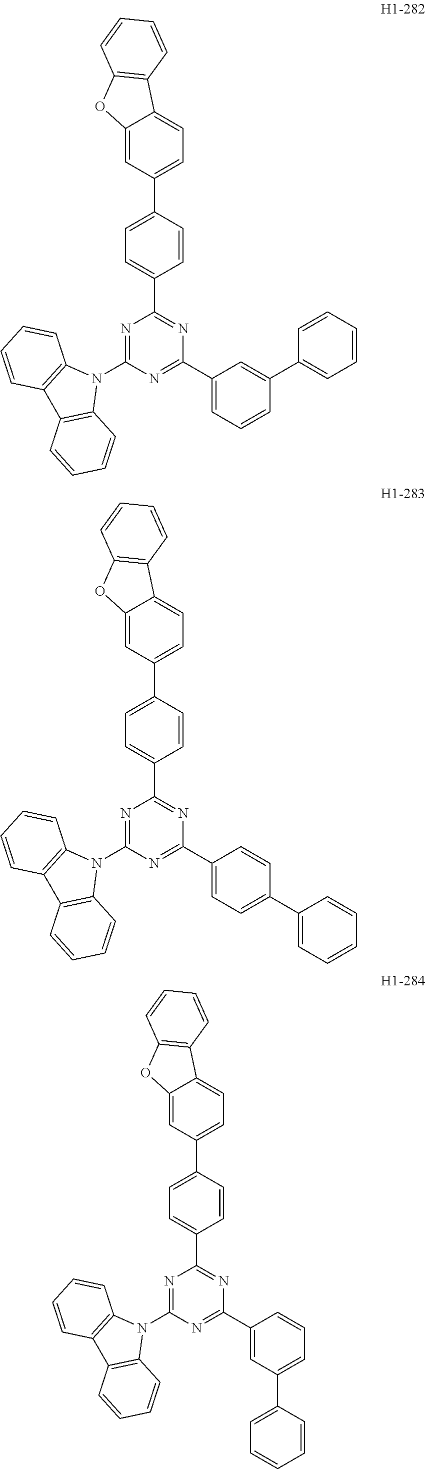

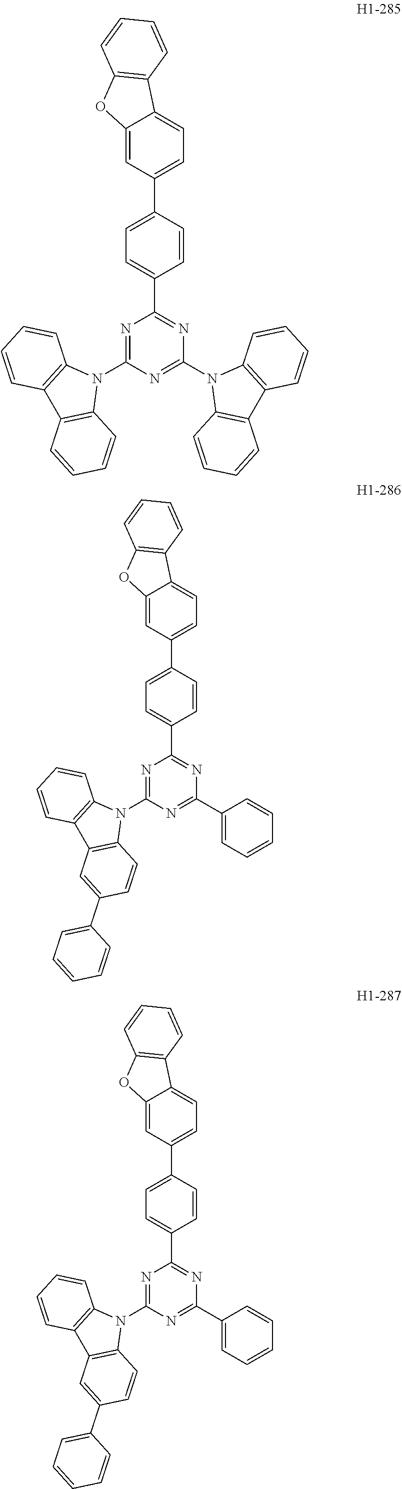 [SMILES: c1ccc2c(c1)oc1cc(-c3ccc(-c4nc(-n5c6ccccc6c6ccccc65)nc(-n5c6ccccc6c6ccccc65)n4)cc3)ccc12 ; c1ccc(-c2ccc3c(c2)c2ccccc2n3-c2nc(-c3ccccc3)nc(-c3ccc(-c4ccc5c(c4)oc4ccccc45)cc3)n2)cc1 ; c1ccc(-c2ccc3c(c2)c2ccccc2n3-c2nc(-c3ccccc3)nc(-c3ccc(-c4ccc5c(c4)oc4ccccc45)cc3)n2)cc1]

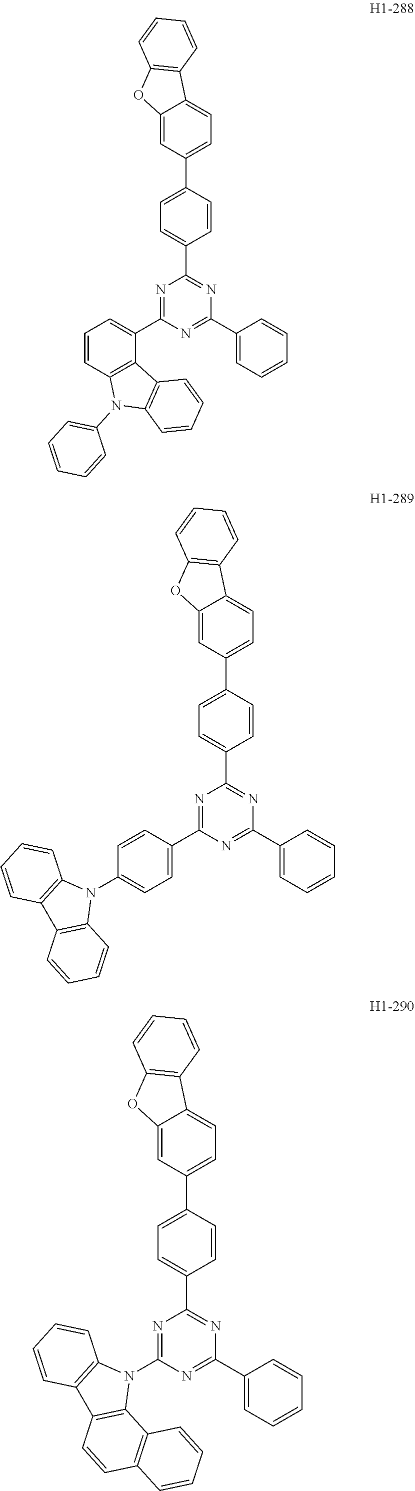 [SMILES: c1ccc(-c2nc(-c3ccc(-c4ccc5c(c4)oc4ccccc45)cc3)nc(-c3cccc4c3c3ccccc3n4-c3ccccc3)n2)cc1 ; c1ccc(-c2nc(-c3ccc(-c4ccc5c(c4)oc4ccccc45)cc3)nc(-c3ccc(-n4c5ccccc5c5ccccc54)cc3)n2)cc1 ; c1ccc(-c2nc(-c3ccc(-c4ccc5c(c4)oc4ccccc45)cc3)nc(-n3c4ccccc4c4ccc5ccccc5c43)n2)cc1]

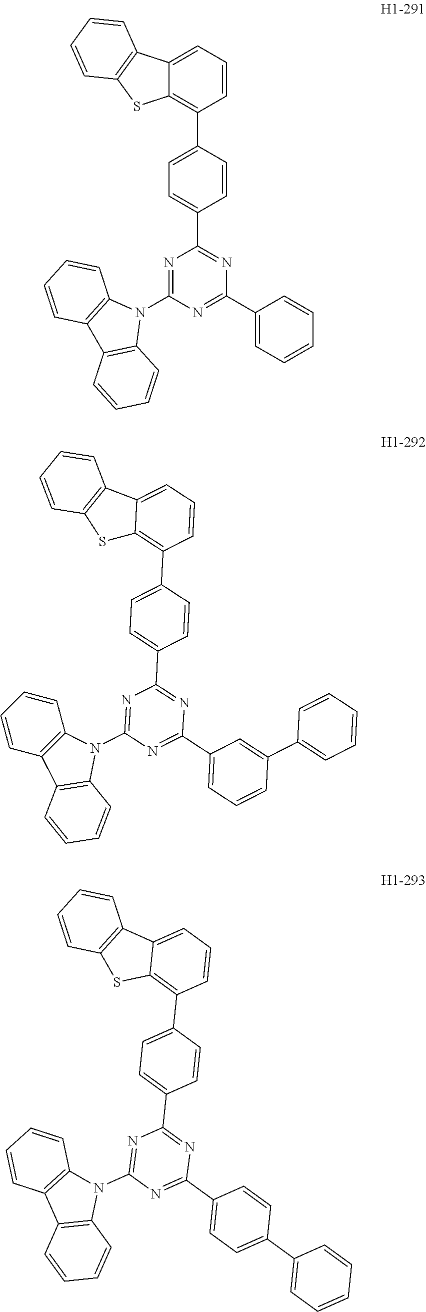 [SMILES: c1ccc(-c2nc(-c3ccc(-c4cccc5c4sc4ccccc45)cc3)nc(-n3c4ccccc4c4ccccc43)n2)cc1 ; c1ccc(-c2cccc(-c3nc(-c4ccc(-c5cccc6c5sc5ccccc56)cc4)nc(-n4c5ccccc5c5ccccc54)n3)c2)cc1 ; c1ccc(-c2ccc(-c3nc(-c4ccc(-c5cccc6c5sc5ccccc56)cc4)nc(-n4c5ccccc5c5ccccc54)n3)cc2)cc1]

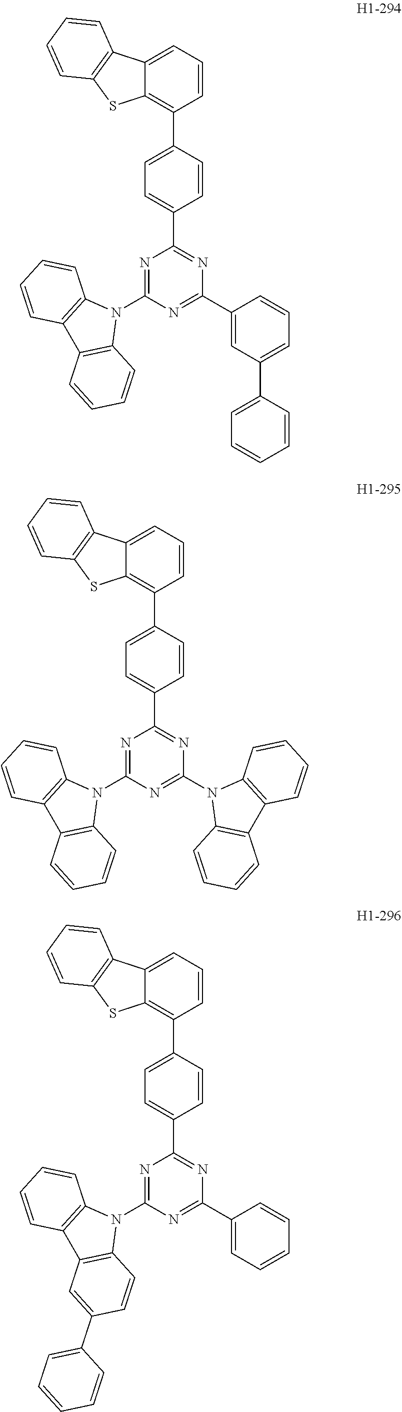

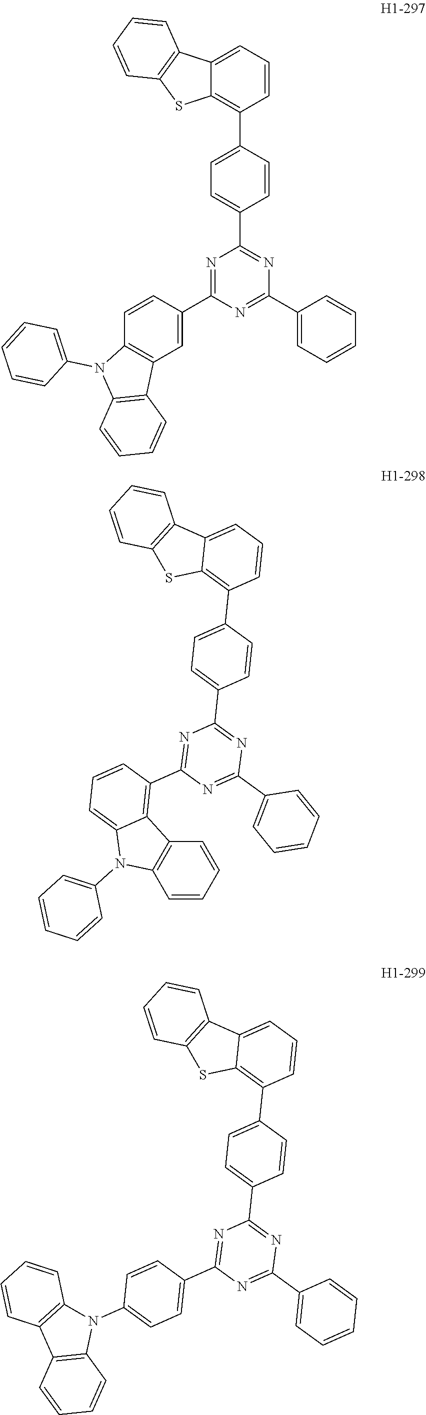

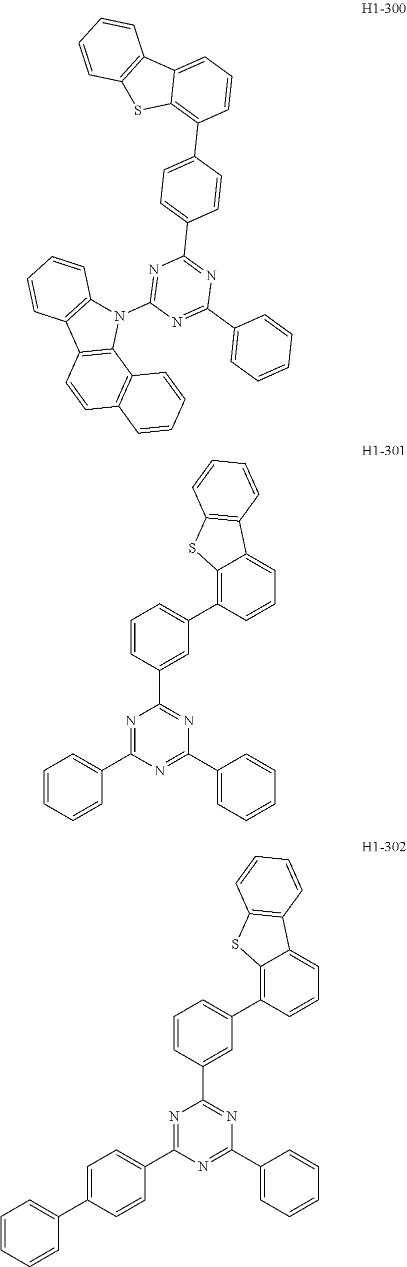

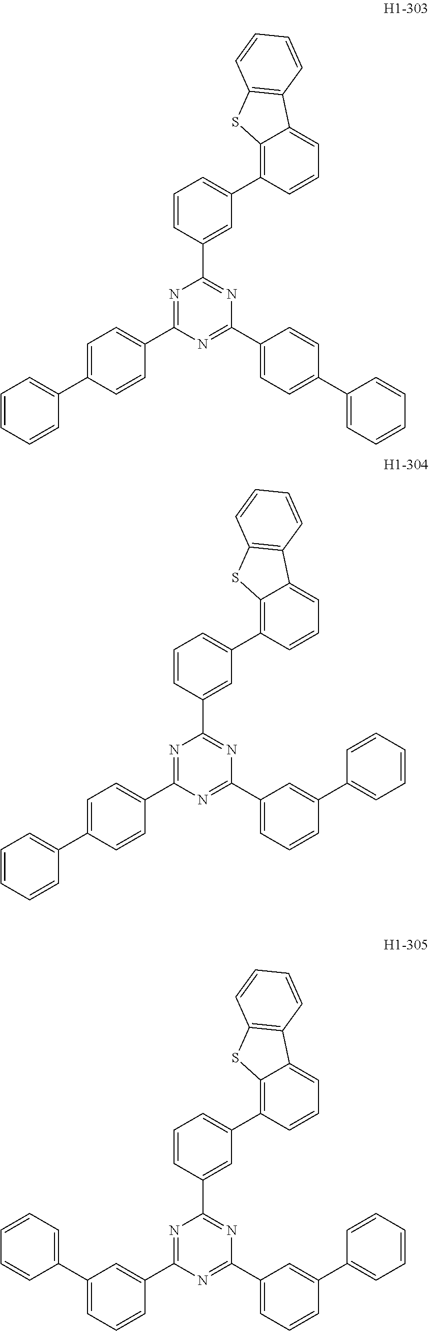 [SMILES: c1ccc(-c2ccc(-c3nc(-c4ccc(-c5ccccc5)cc4)nc(-c4cccc(-c5cccc6c5sc5ccccc56)c4)n3)cc2)cc1 ; c1ccc(-c2ccc(-c3nc(-c4cccc(-c5ccccc5)c4)nc(-c4cccc(-c5cccc6c5sc5ccccc56)c4)n3)cc2)cc1 ; c1ccc(-c2cccc(-c3nc(-c4cccc(-c5ccccc5)c4)nc(-c4cccc(-c5cccc6c5sc5ccccc56)c4)n3)c2)cc1]

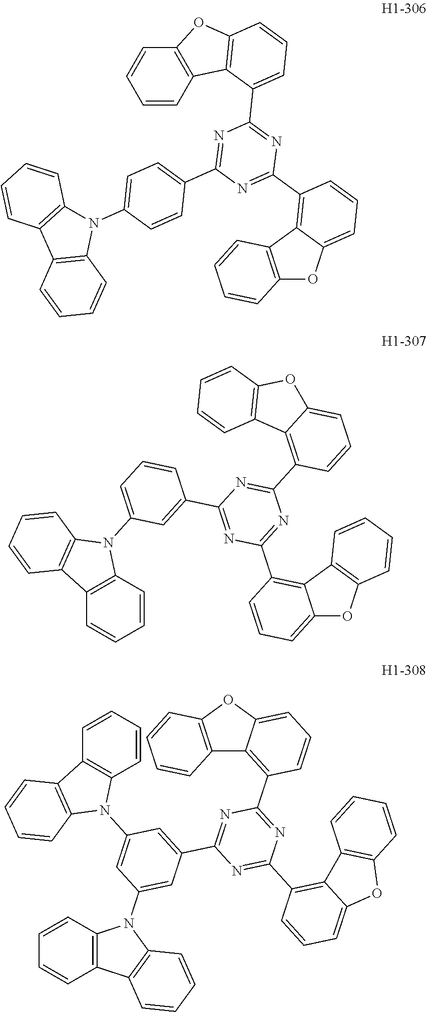 [SMILES: c1ccc2c(c1)oc1cccc(-c3nc(-c4ccc(-n5c6ccccc6c6ccccc65)cc4)nc(-c4cccc5oc6ccccc6c45)n3)c12 ; c1cc(-c2nc(-c3cccc4oc5ccccc5c34)nc(-c3cccc4oc5ccccc5c34)n2)cc(-n2c3ccccc3c3ccccc32)c1 ; c1ccc2c(c1)oc1cccc(-c3nc(-c4cc(-n5c6ccccc6c6ccccc65)cc(-n5c6ccccc6c6ccccc65)c4)nc(-c4cccc5oc6ccccc6c45)n3)c12]

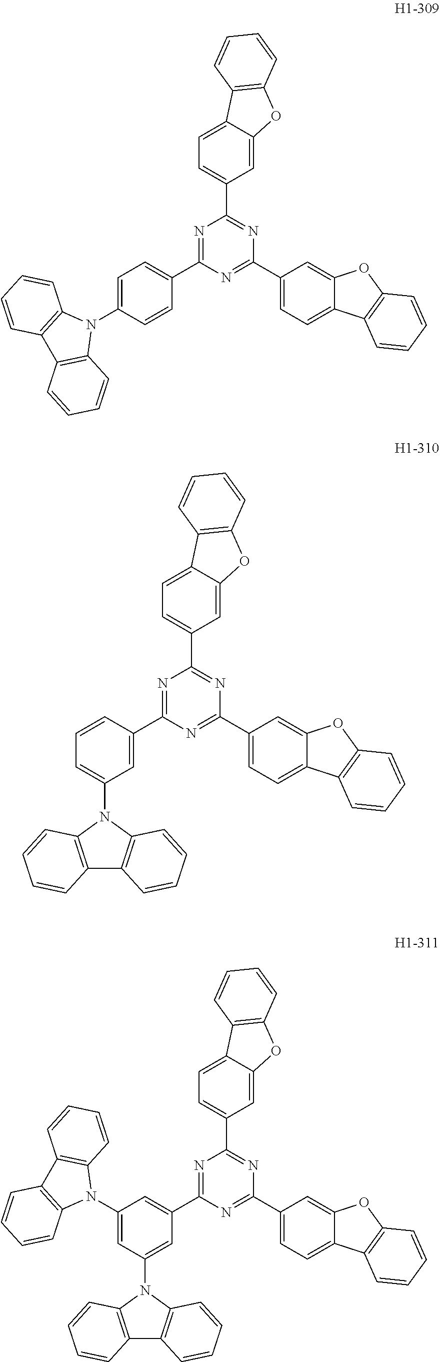 [SMILES: c1ccc2c(c1)oc1cc(-c3nc(-c4ccc(-n5c6ccccc6c6ccccc65)cc4)nc(-c4ccc5c(c4)oc4ccccc45)n3)ccc12 ; c1cc(-c2nc(-c3ccc4c(c3)oc3ccccc34)nc(-c3ccc4c(c3)oc3ccccc34)n2)cc(-n2c3ccccc3c3ccccc32)c1 ; c1ccc2c(c1)oc1cc(-c3nc(-c4cc(-n5c6ccccc6c6ccccc65)cc(-n5c6ccccc6c6ccccc65)c4)nc(-c4ccc5c(c4)oc4ccccc45)n3)ccc12]

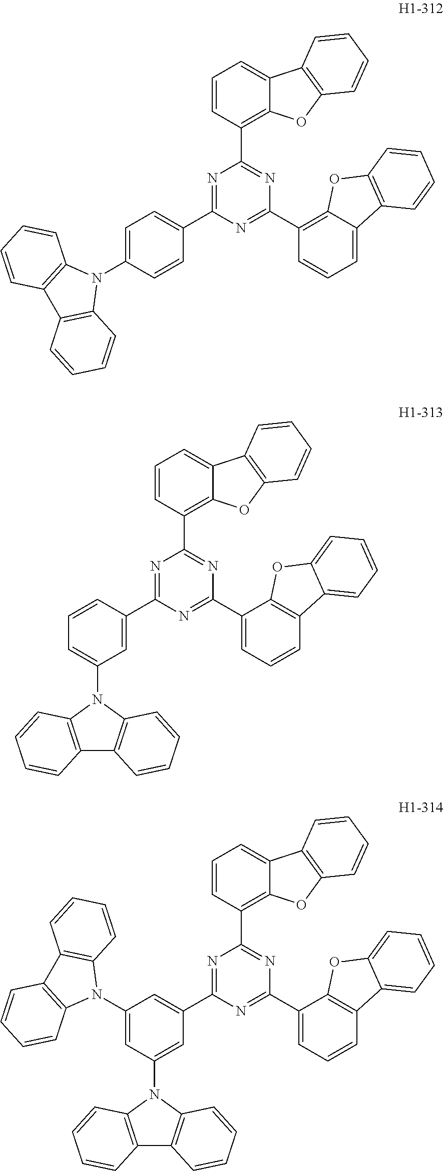 [SMILES: c1ccc2c(c1)oc1c(-c3nc(-c4ccc(-n5c6ccccc6c6ccccc65)cc4)nc(-c4cccc5c4oc4ccccc45)n3)cccc12 ; c1cc(-c2nc(-c3cccc4c3oc3ccccc34)nc(-c3cccc4c3oc3ccccc34)n2)cc(-n2c3ccccc3c3ccccc32)c1 ; c1ccc2c(c1)oc1c(-c3nc(-c4cc(-n5c6ccccc6c6ccccc65)cc(-n5c6ccccc6c6ccccc65)c4)nc(-c4cccc5c4oc4ccccc45)n3)cccc12]

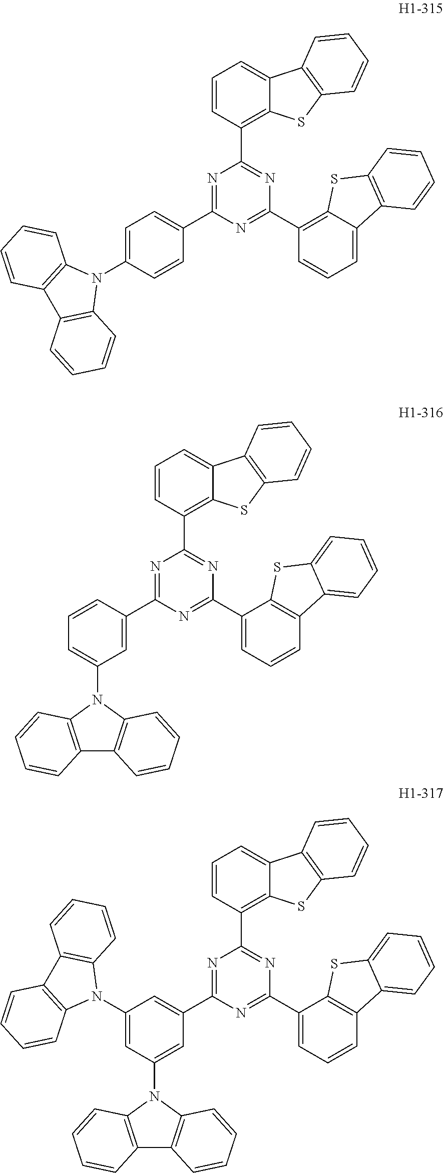 [SMILES: c1ccc2c(c1)sc1c(-c3nc(-c4ccc(-n5c6ccccc6c6ccccc65)cc4)nc(-c4cccc5c4sc4ccccc45)n3)cccc12 ; c1cc(-c2nc(-c3cccc4c3sc3ccccc34)nc(-c3cccc4c3sc3ccccc34)n2)cc(-n2c3ccccc3c3ccccc32)c1 ; c1ccc2c(c1)sc1c(-c3nc(-c4cc(-n5c6ccccc6c6ccccc65)cc(-n5c6ccccc6c6ccccc65)c4)nc(-c4cccc5c4sc4ccccc45)n3)cccc12]

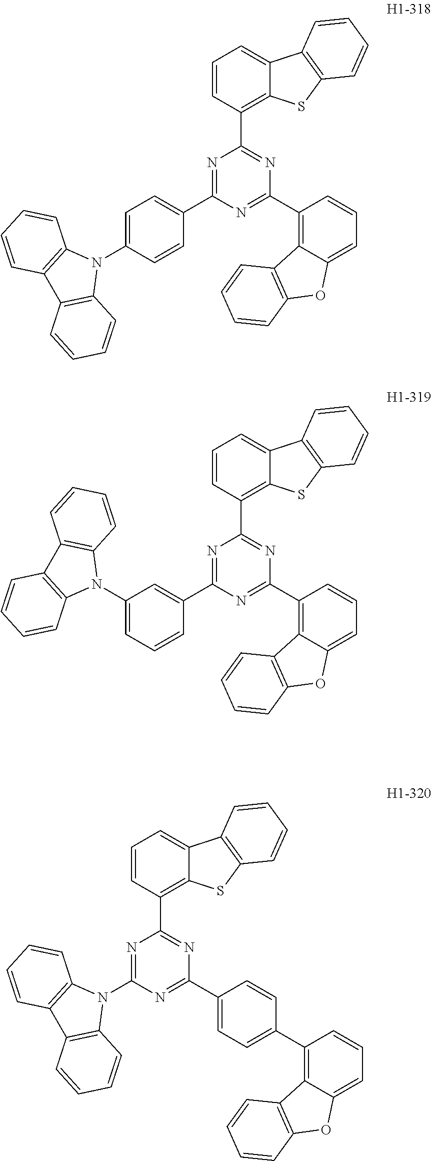 [SMILES: c1ccc2c(c1)oc1cccc(-c3nc(-c4ccc(-n5c6ccccc6c6ccccc65)cc4)nc(-c4cccc5c4sc4ccccc45)n3)c12 ; c1cc(-c2nc(-c3cccc4c3sc3ccccc34)nc(-c3cccc4oc5ccccc5c34)n2)cc(-n2c3ccccc3c3ccccc32)c1 ; c1ccc2c(c1)oc1cccc(-c3ccc(-c4nc(-c5cccc6c5sc5ccccc56)nc(-n5c6ccccc6c6ccccc65)n4)cc3)c12]

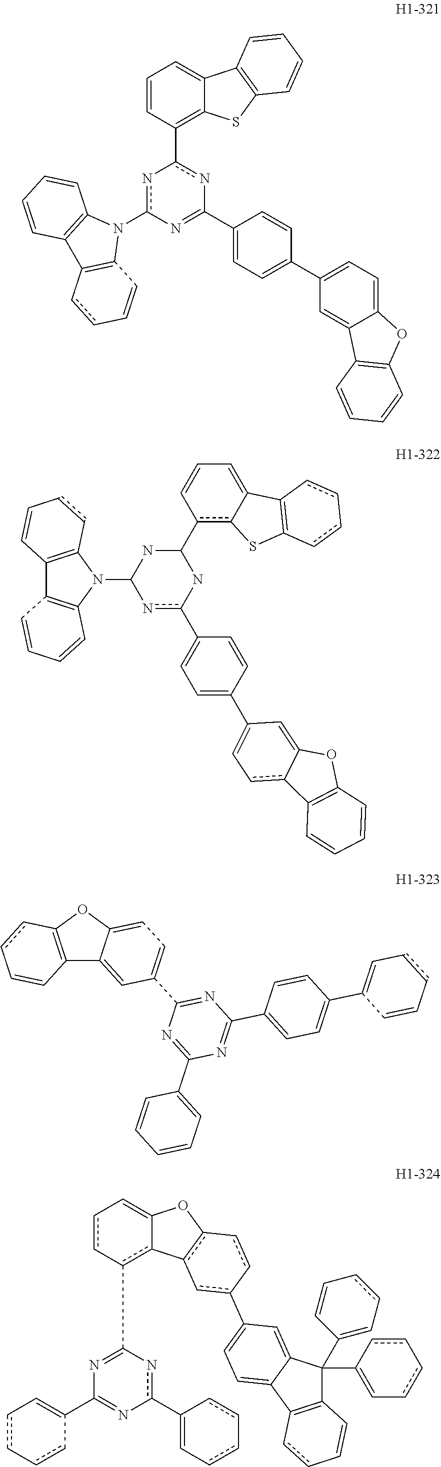

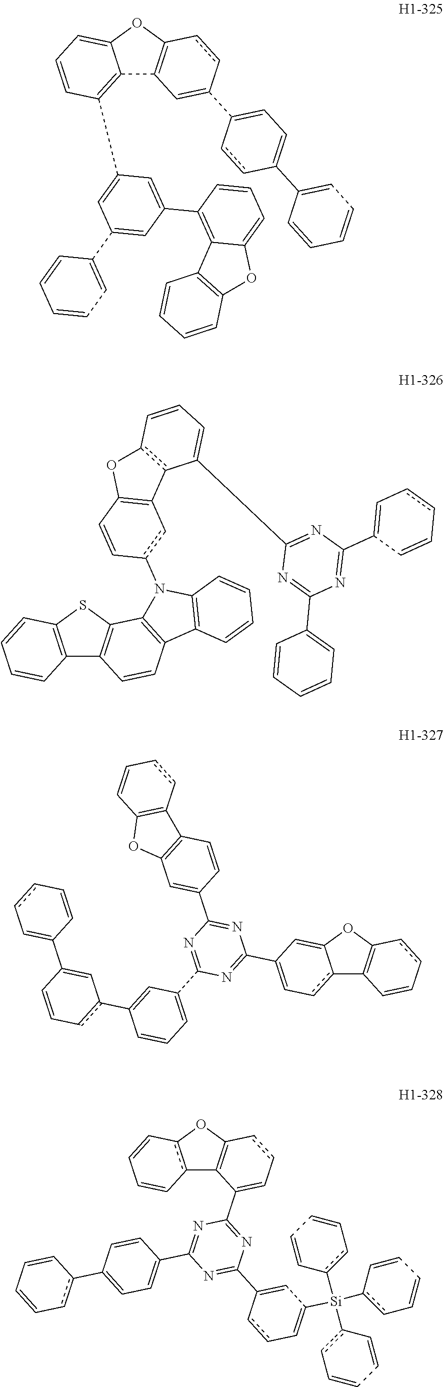

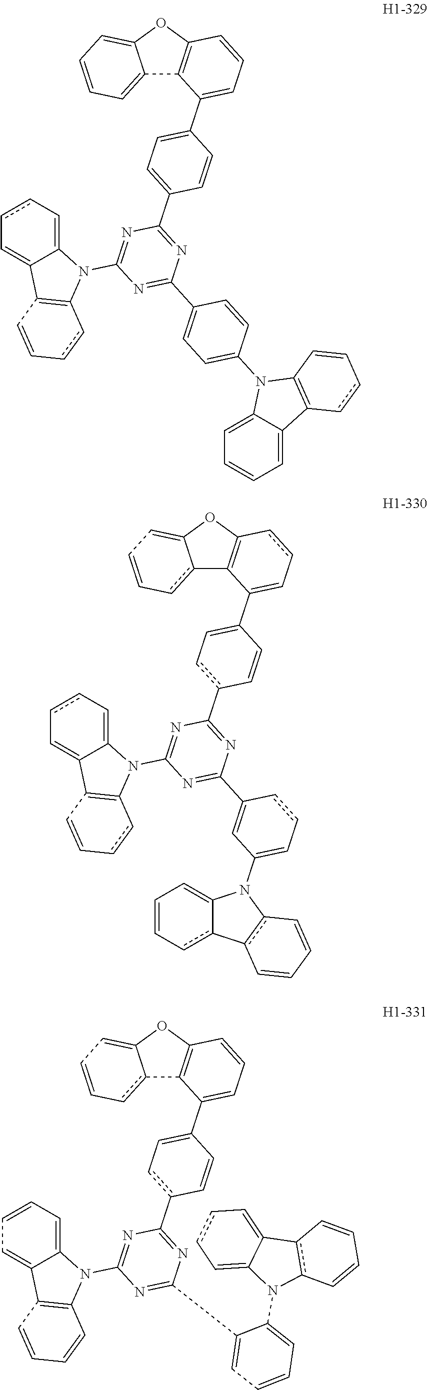

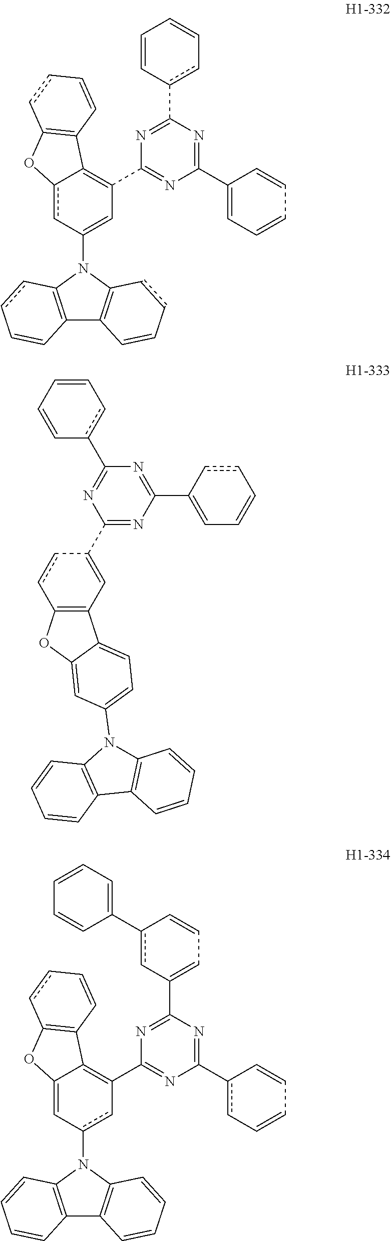

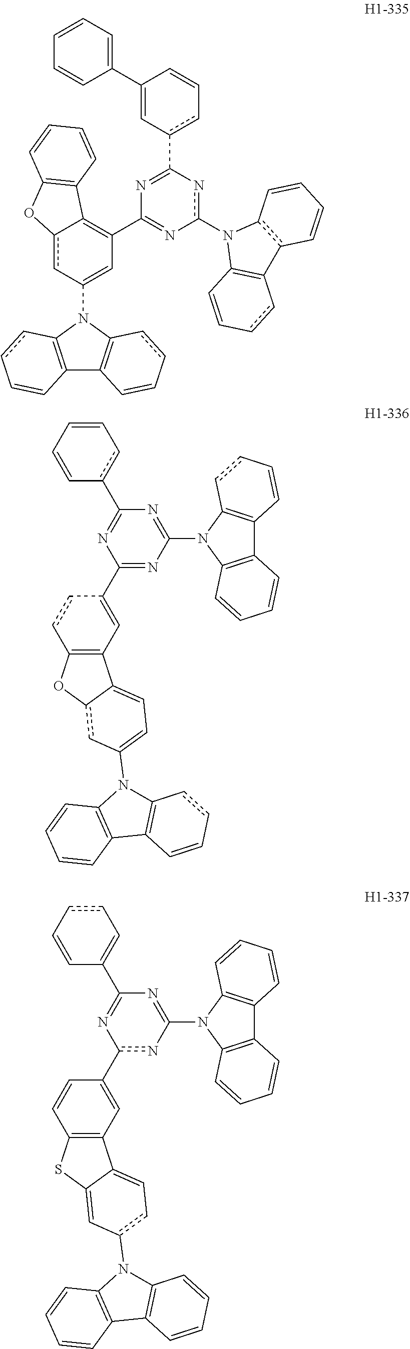

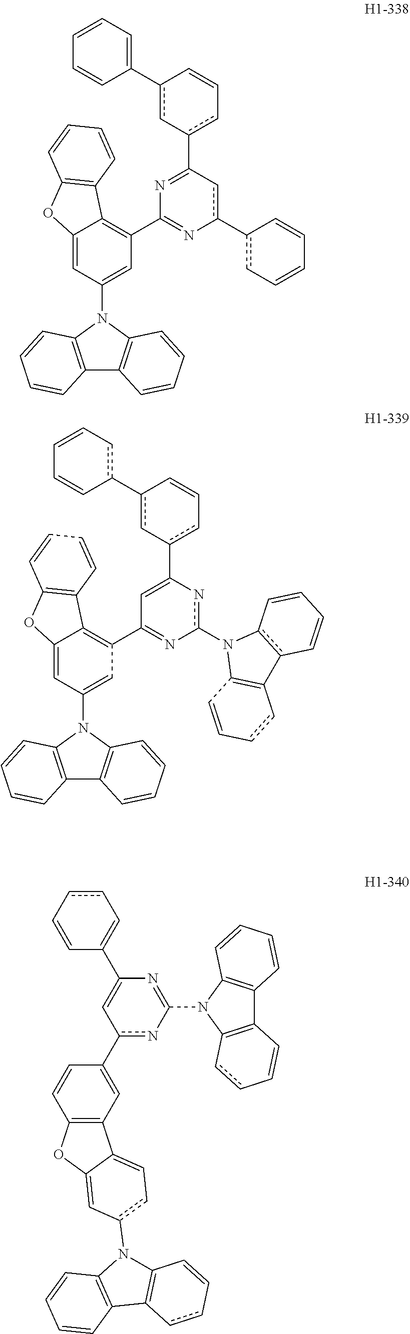

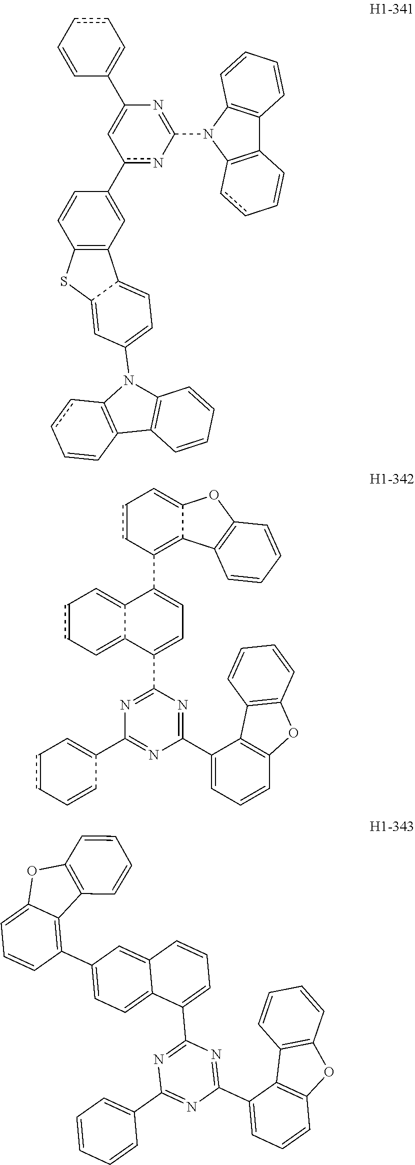

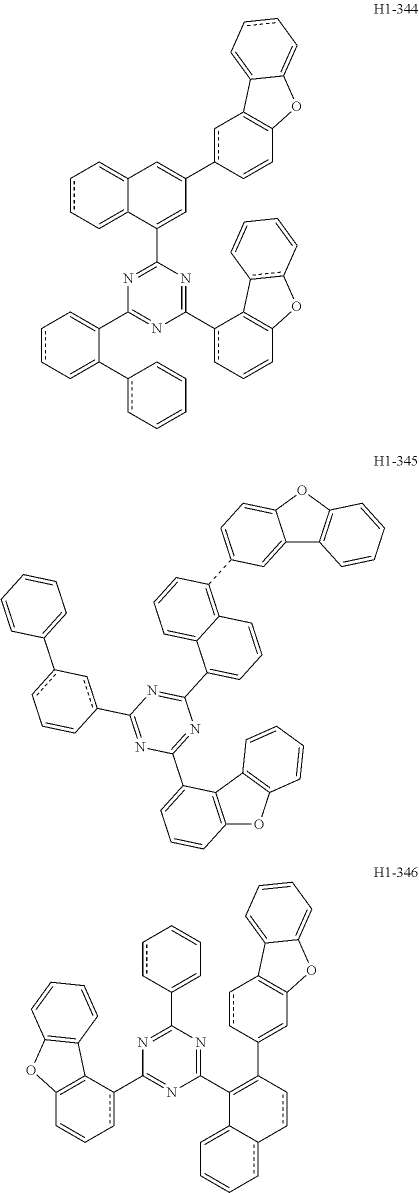

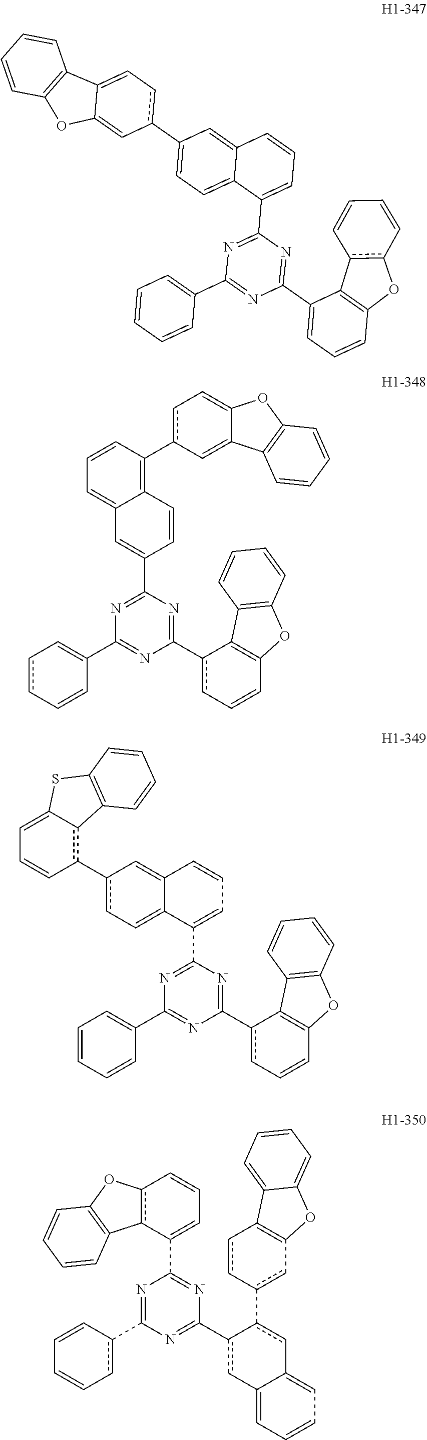

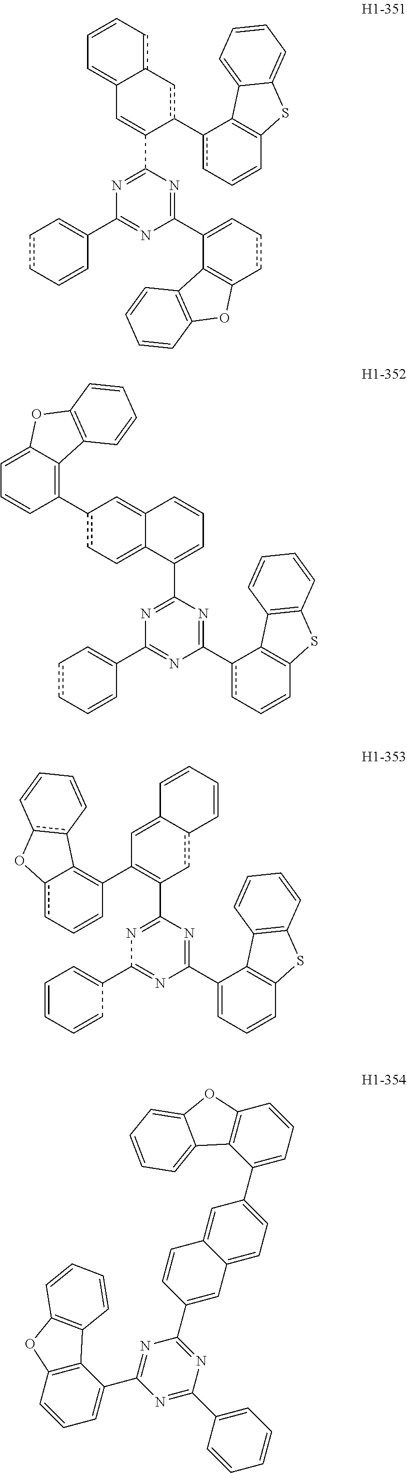

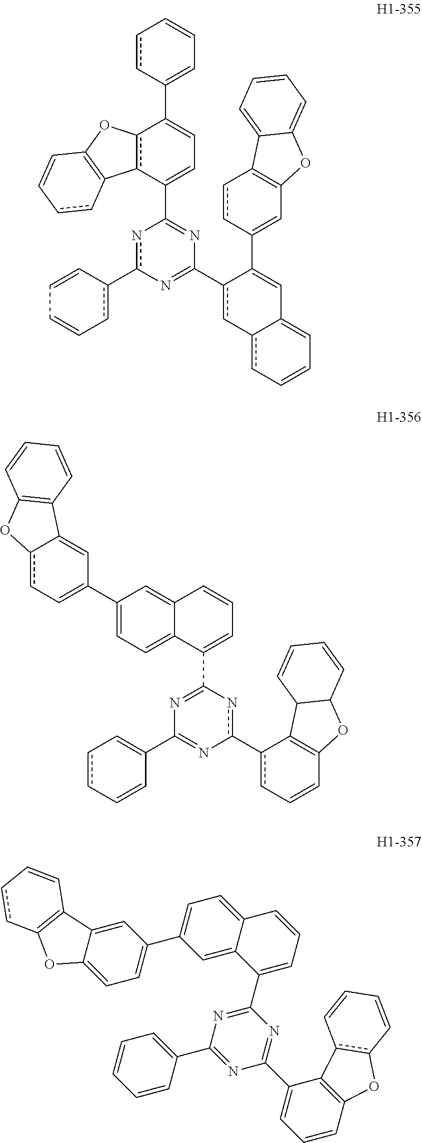

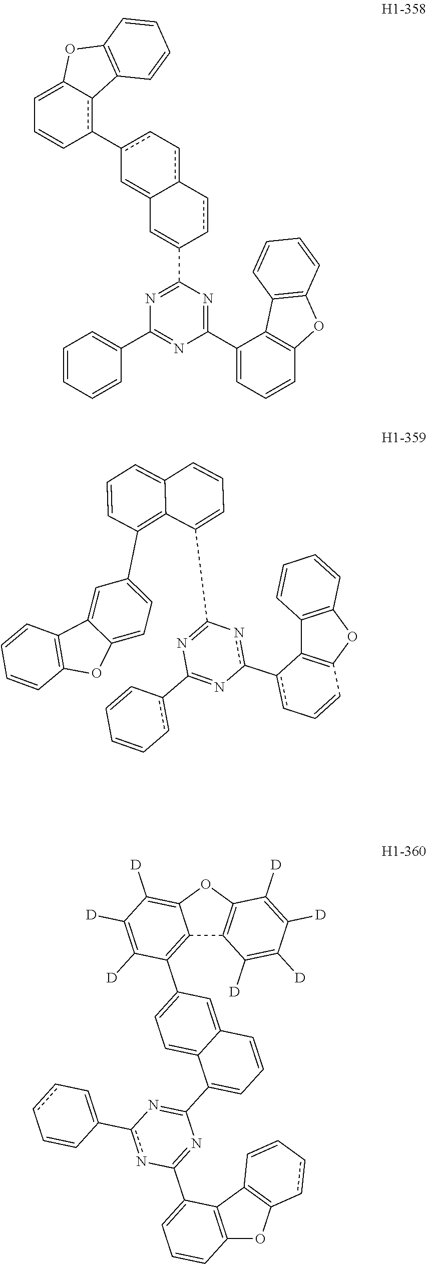

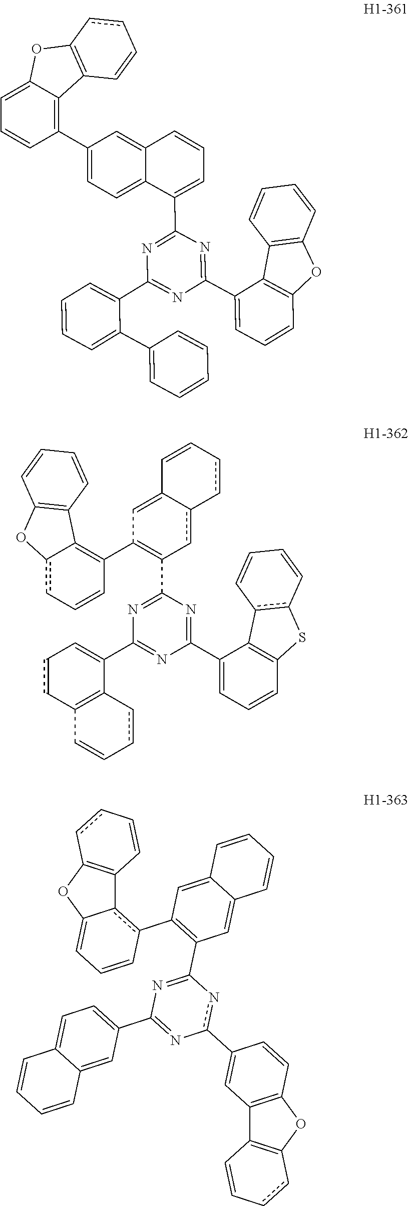

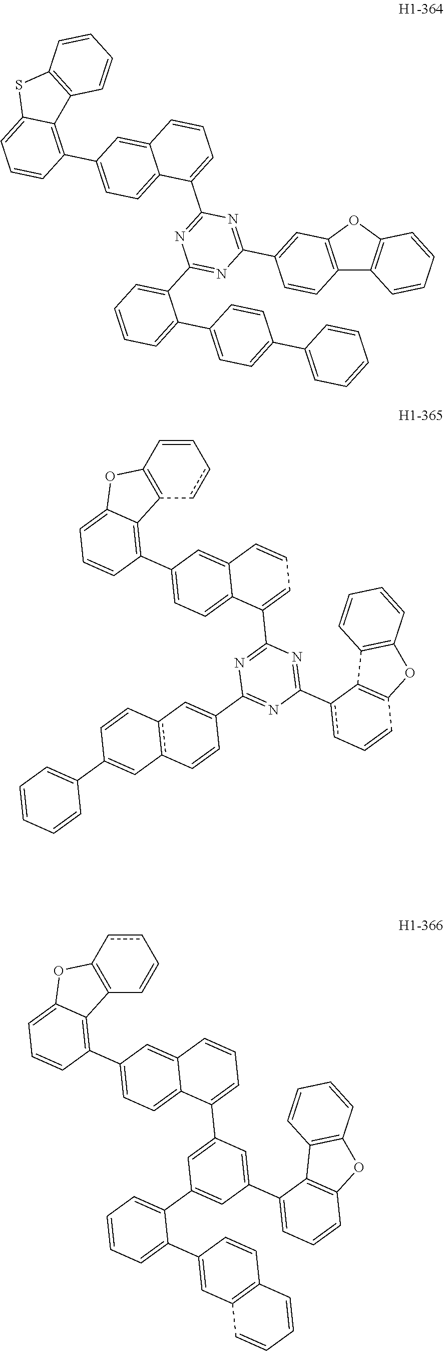

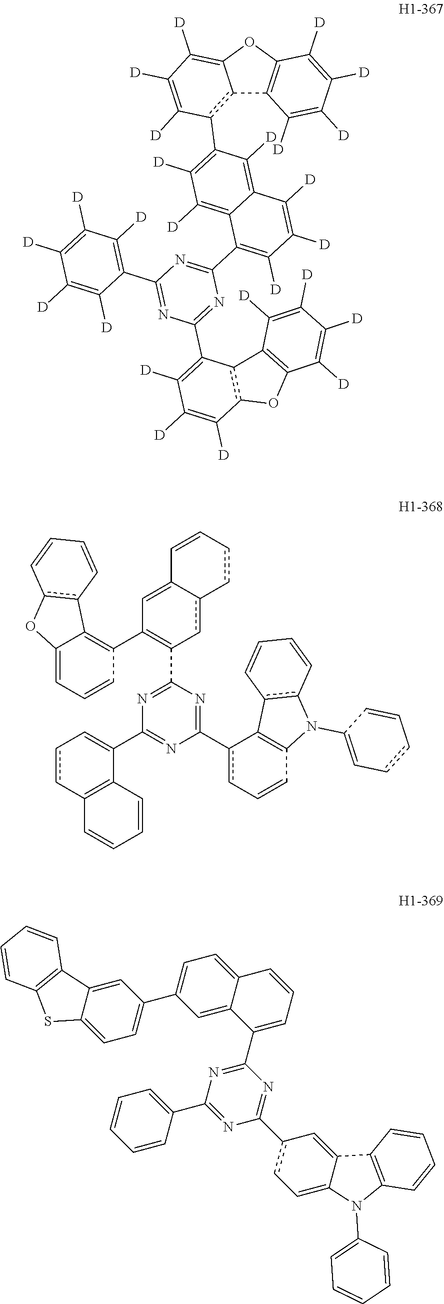

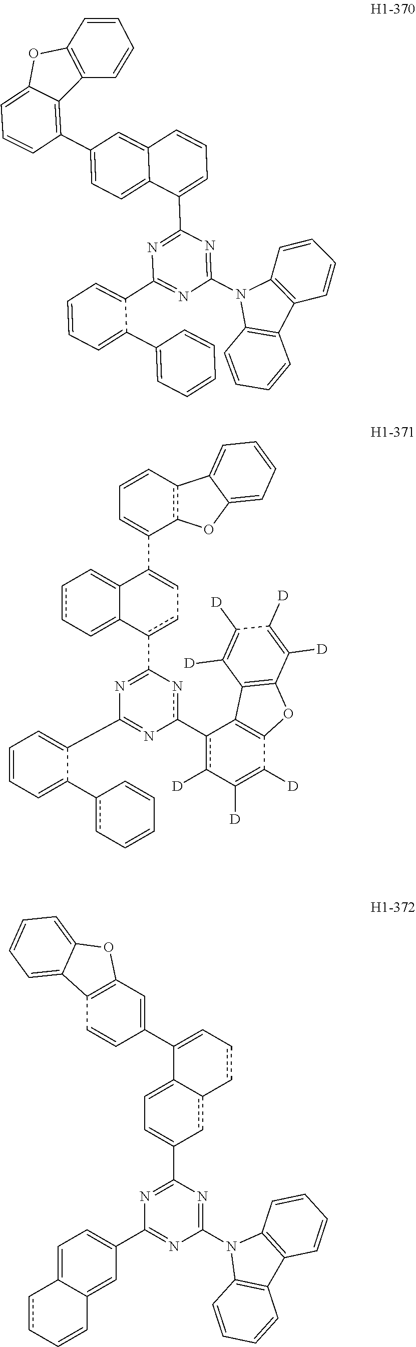

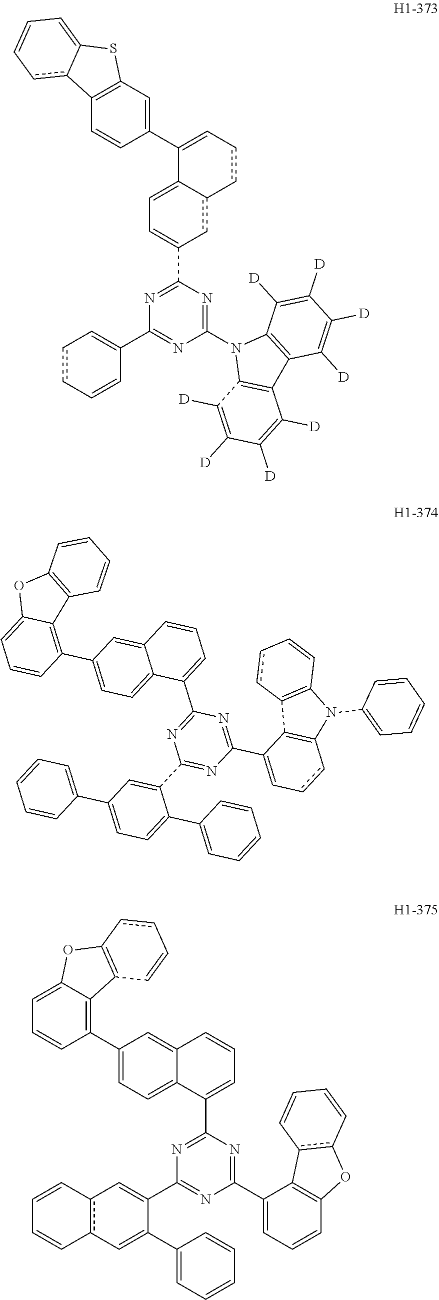

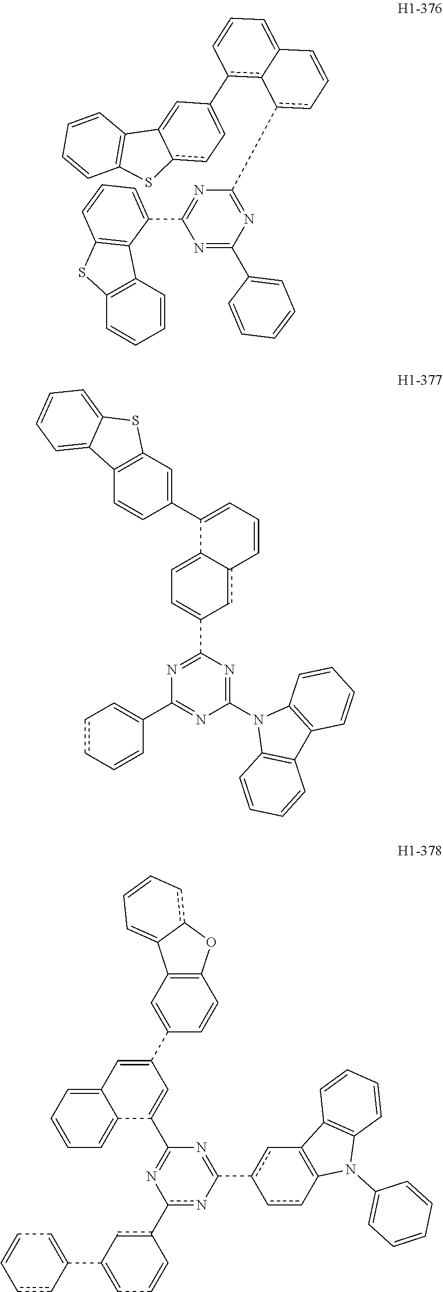

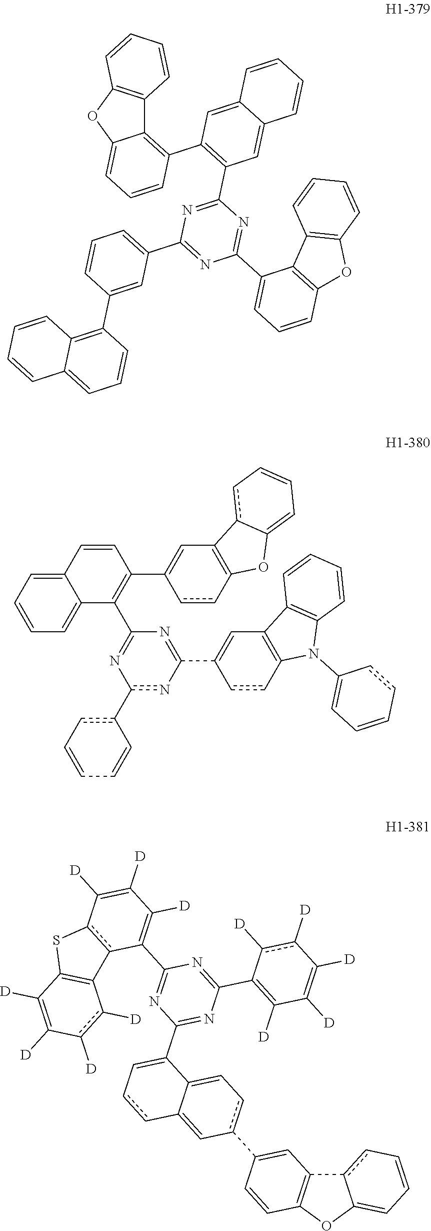

The compound represented by formula 2 may be at least one selected from the group consisting of the following compounds, but is not limited thereto.

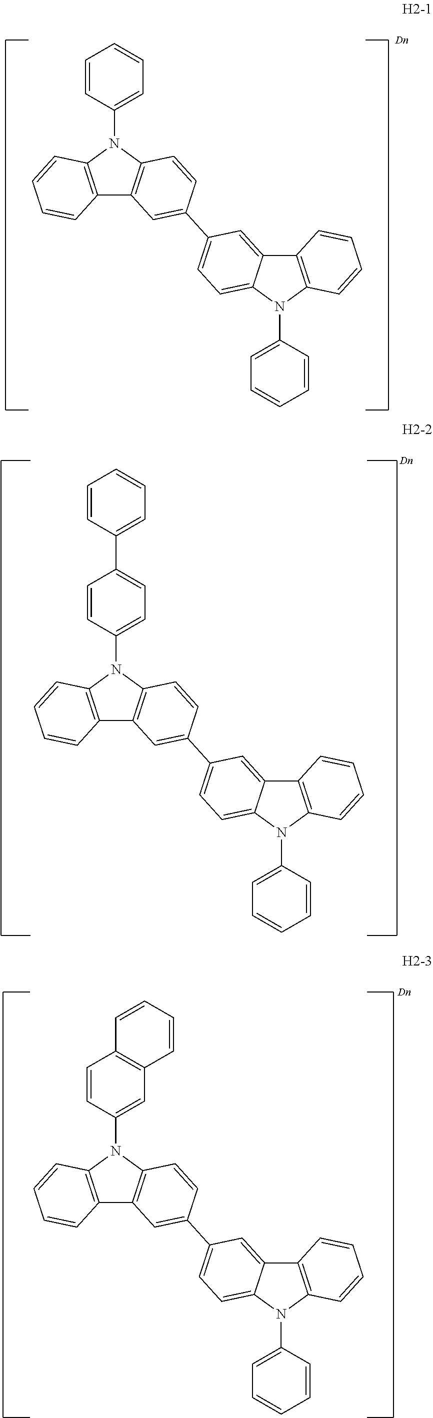

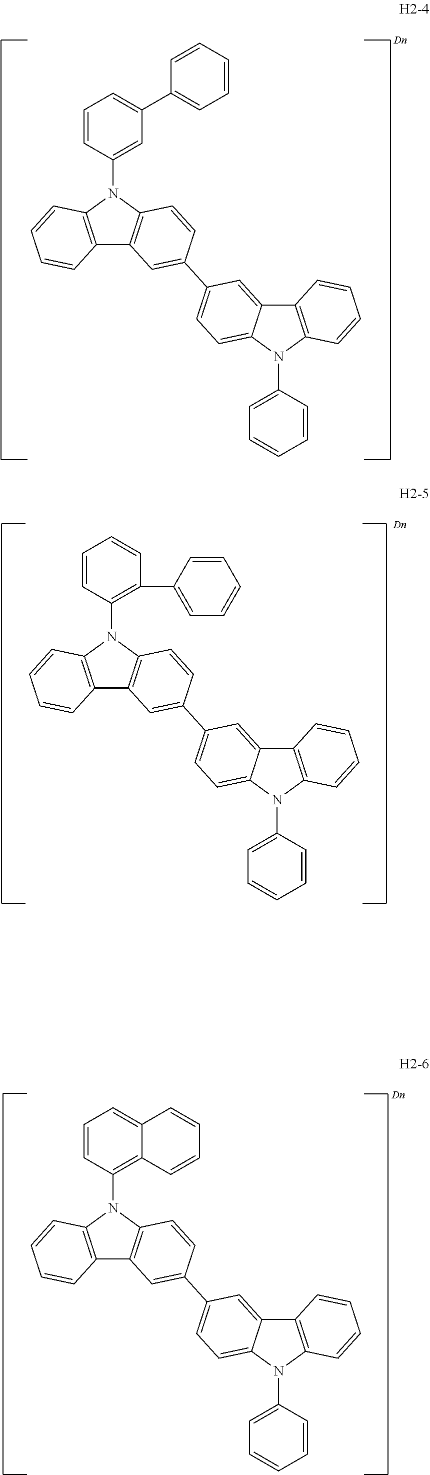

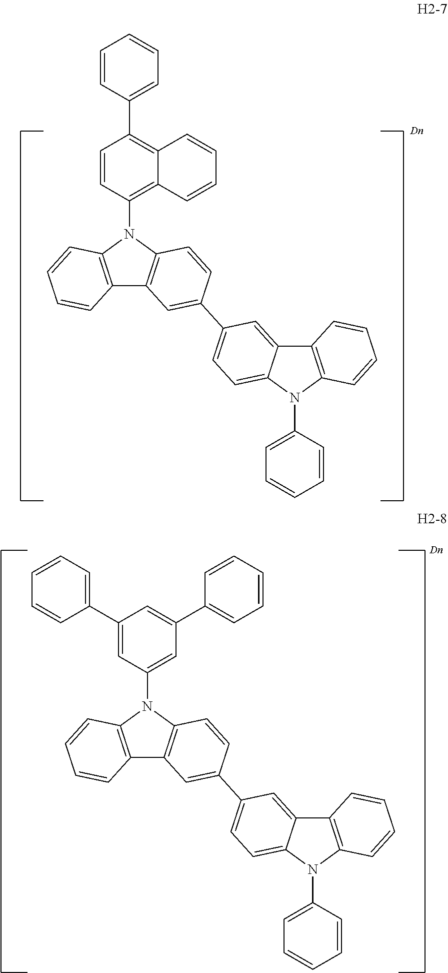

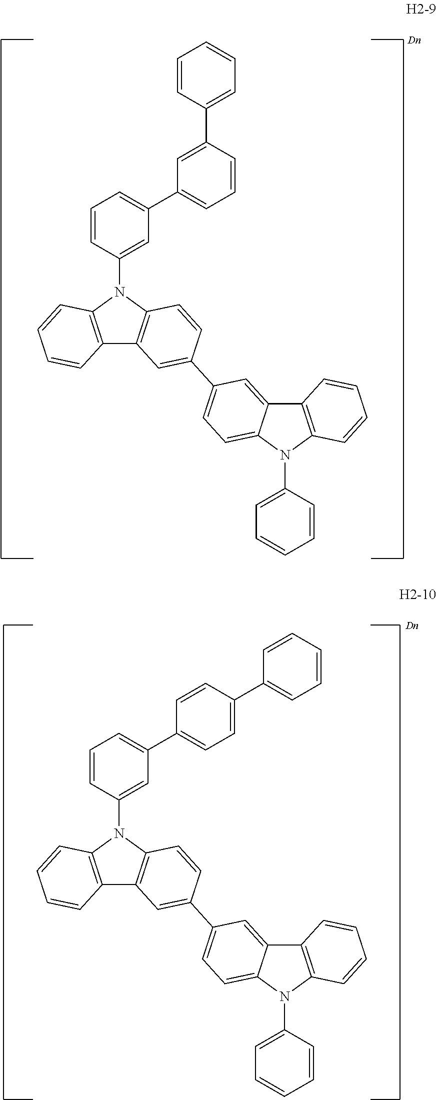

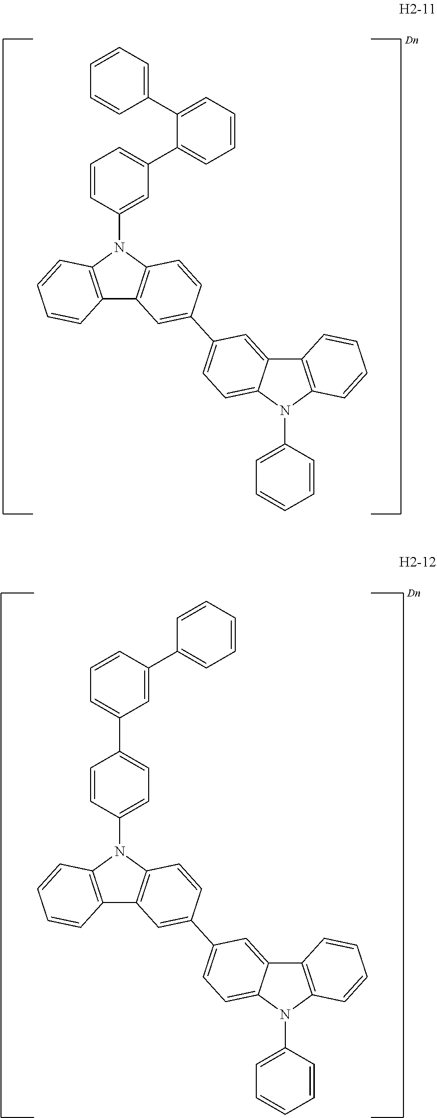

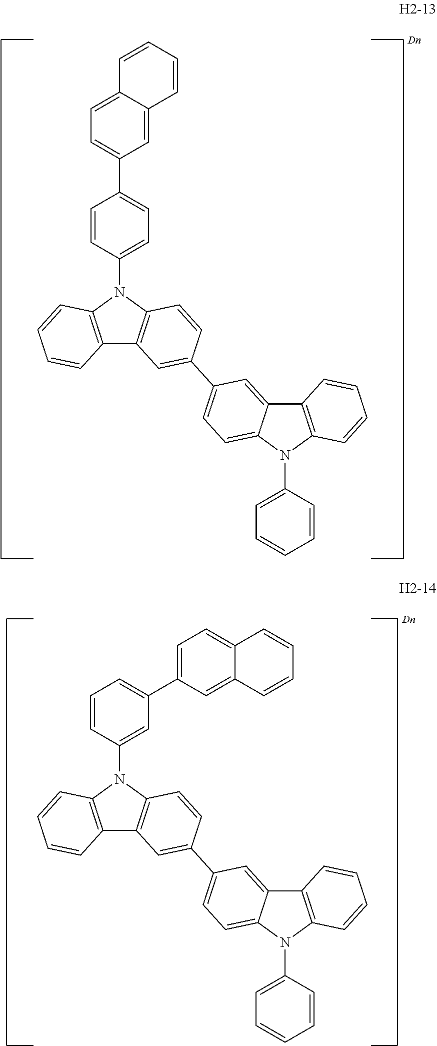

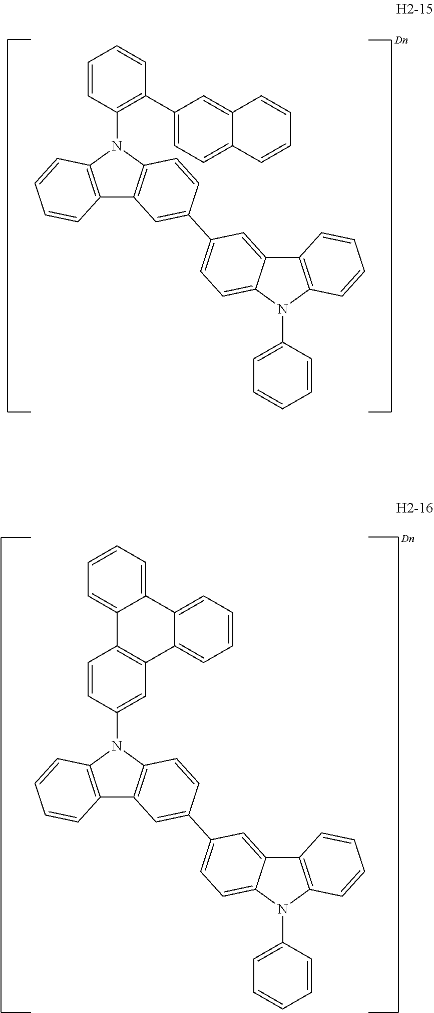

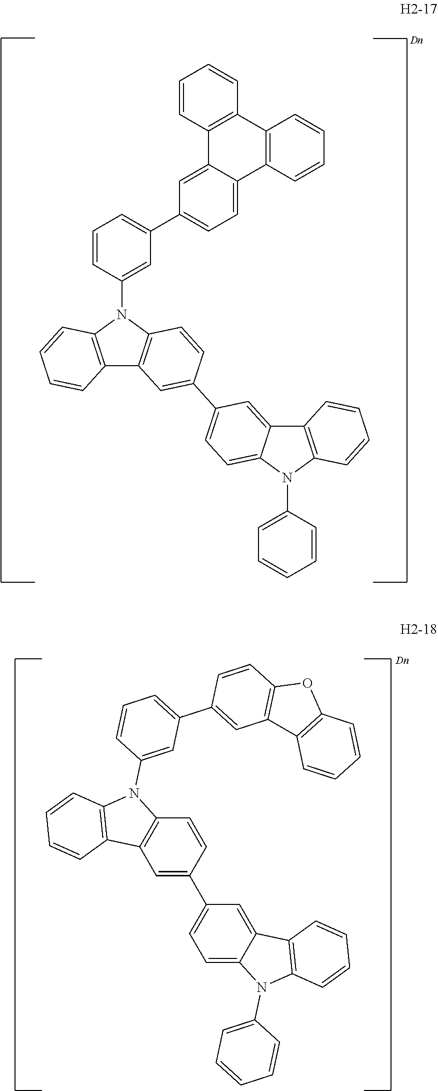

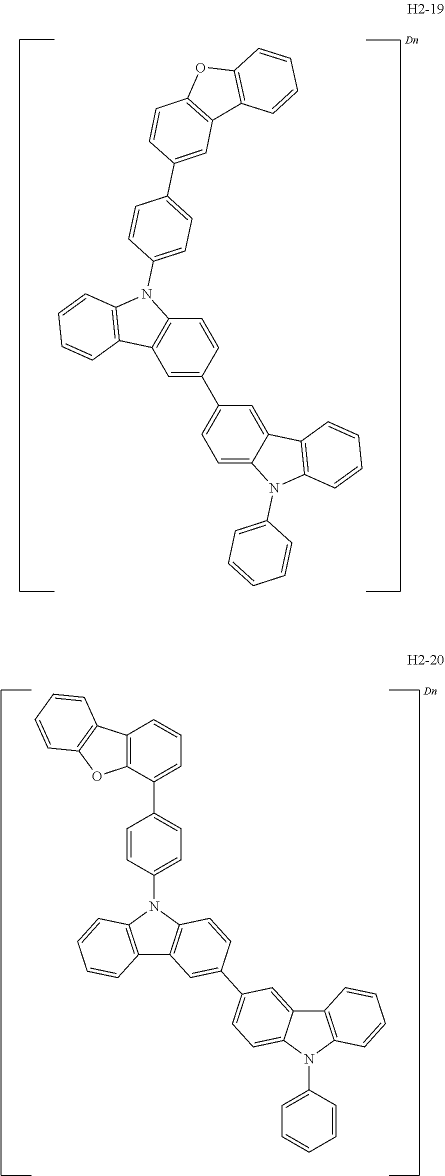

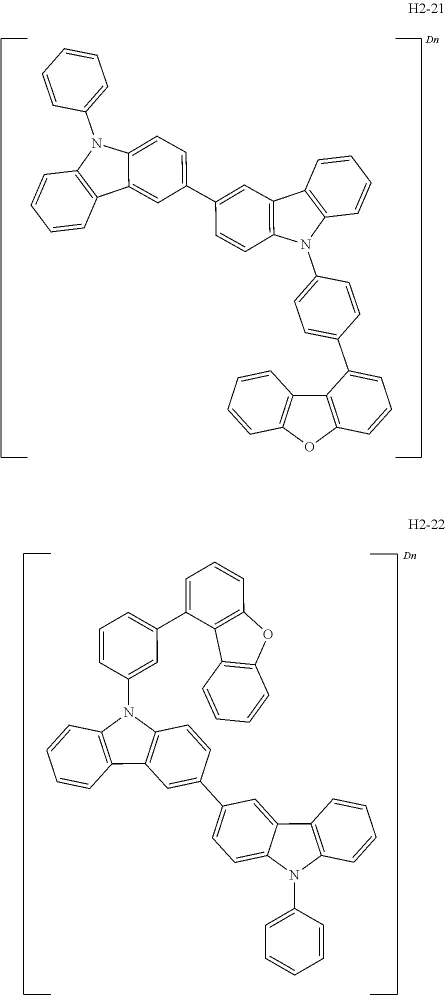

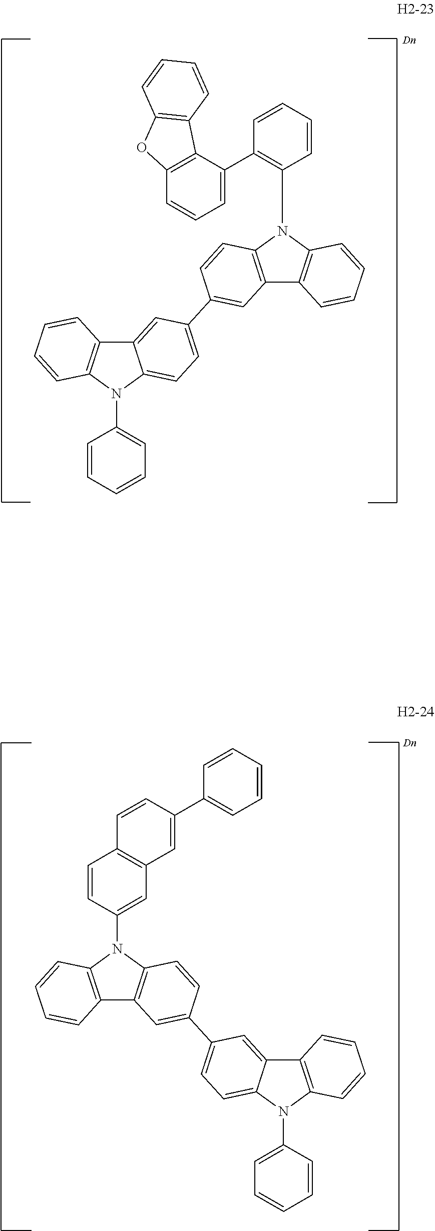

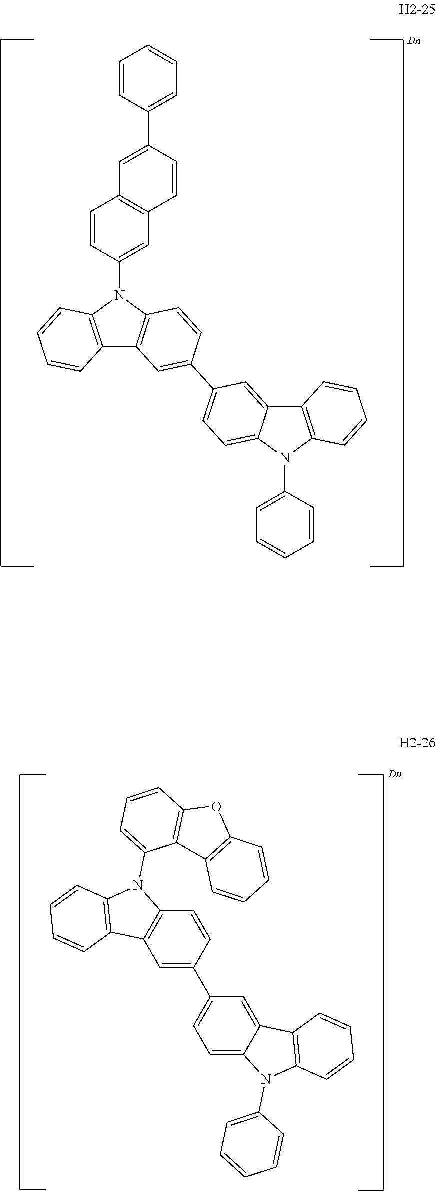

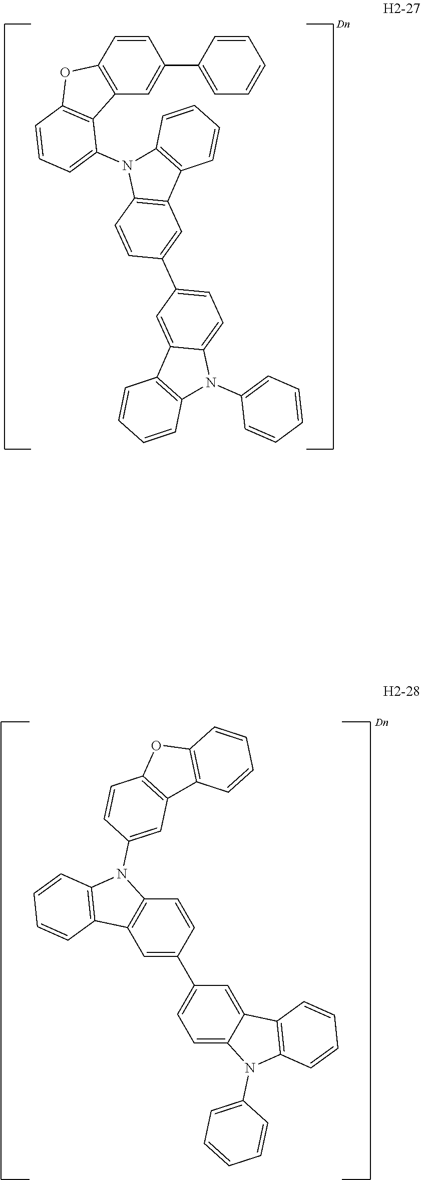

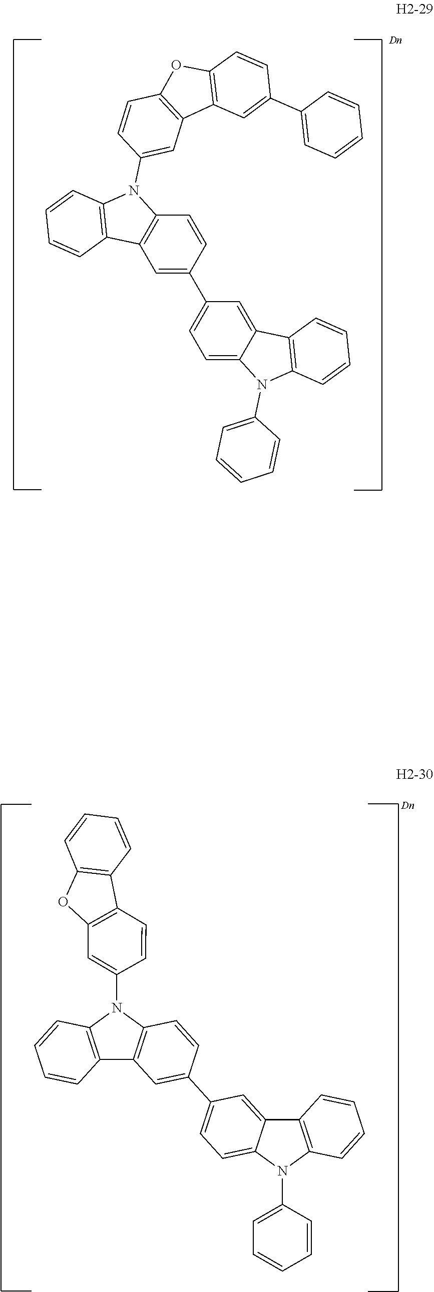

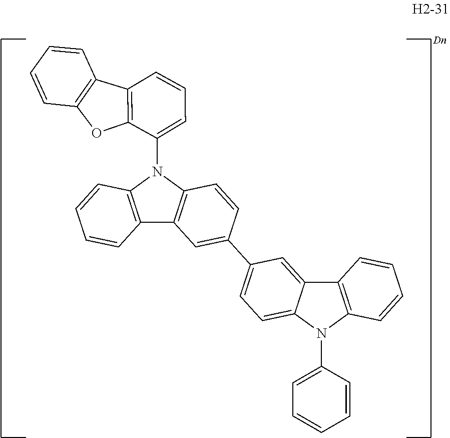

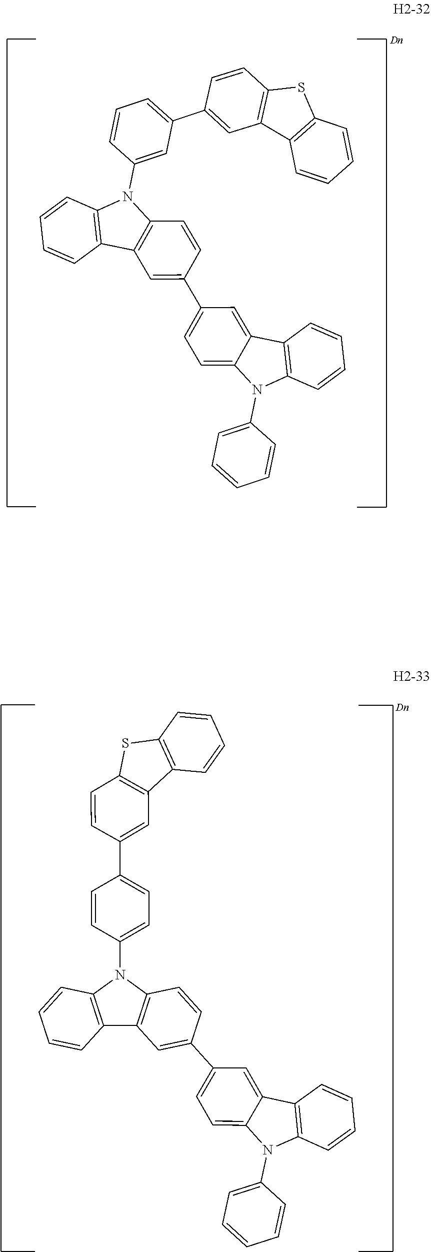

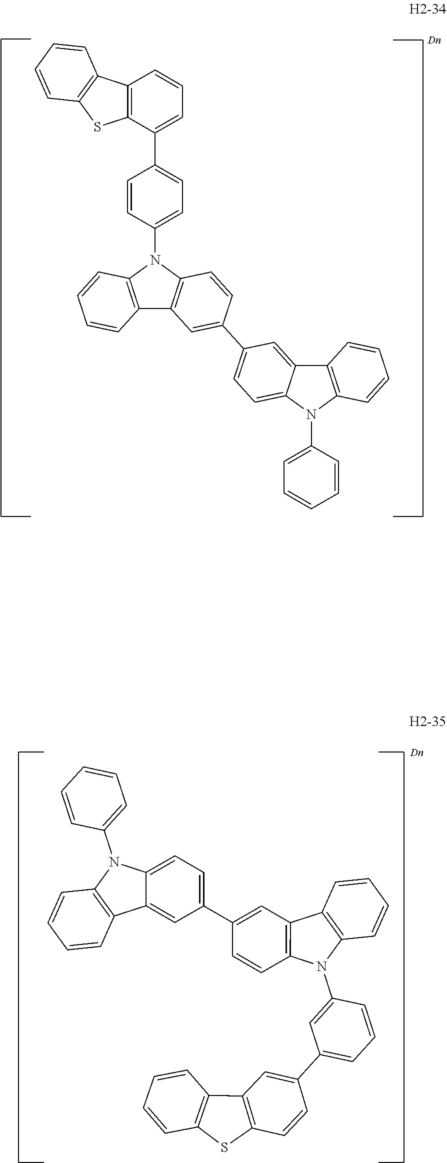

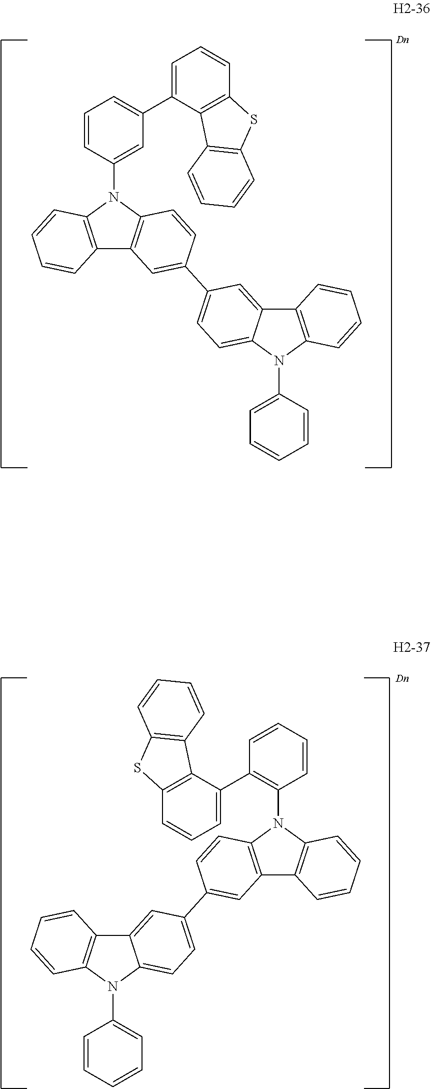

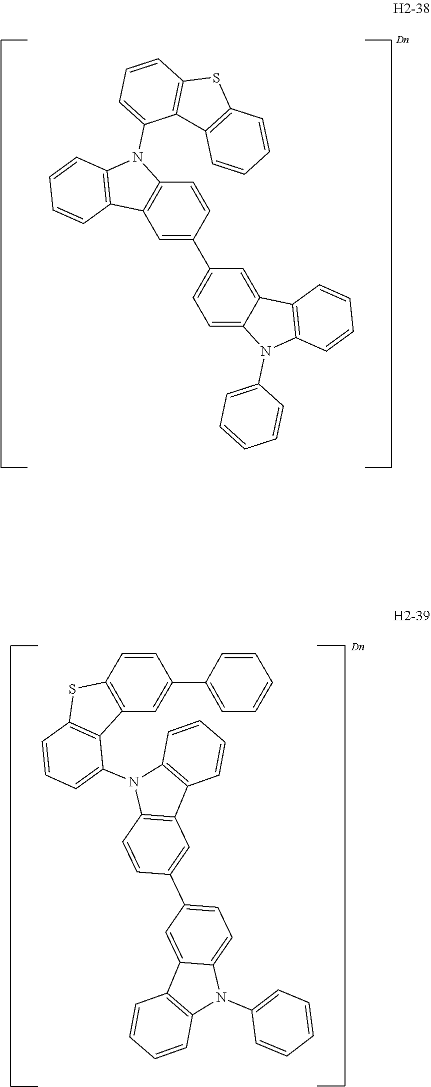

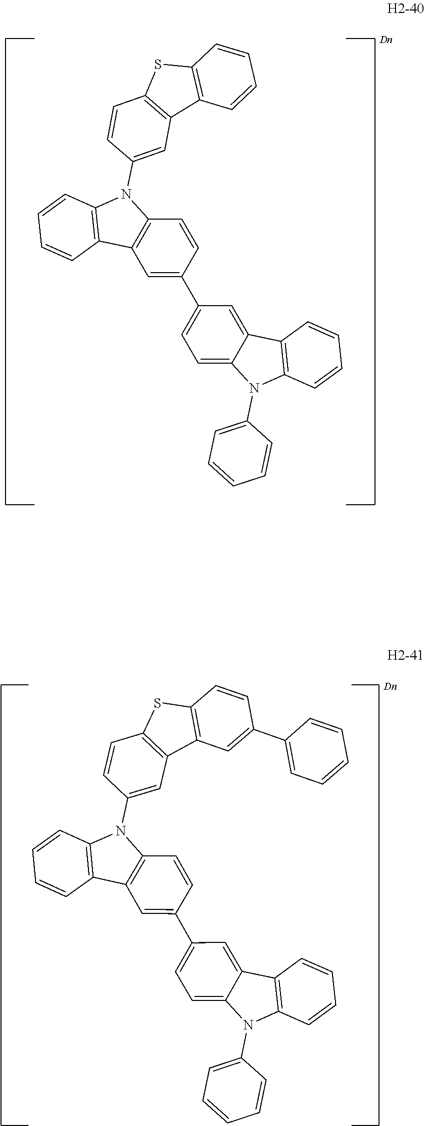

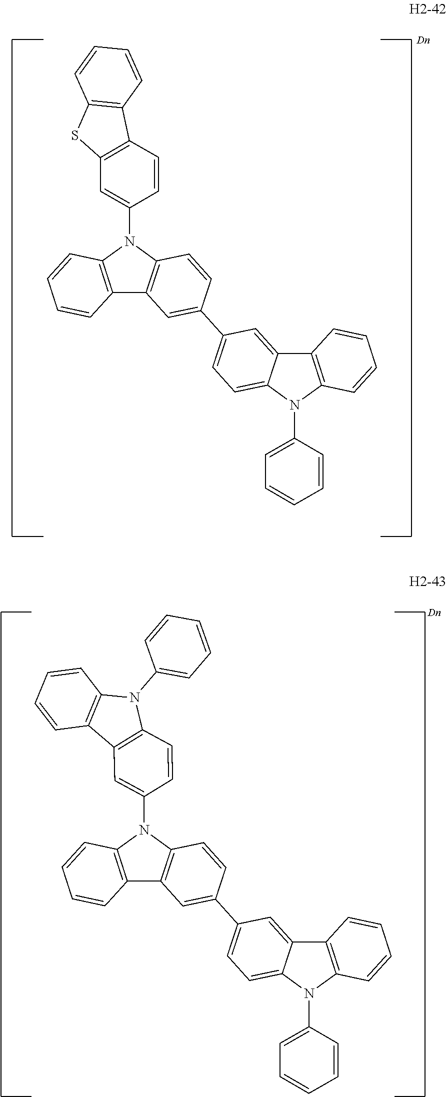

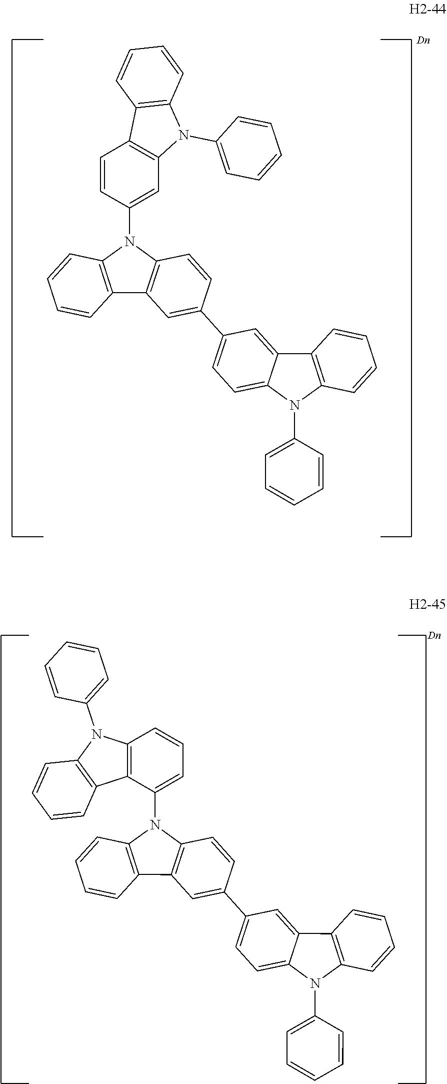

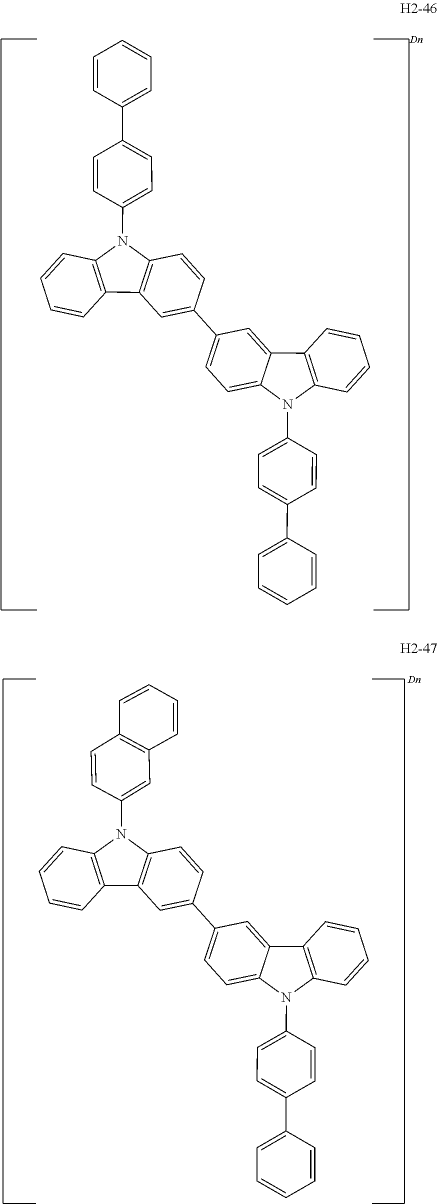

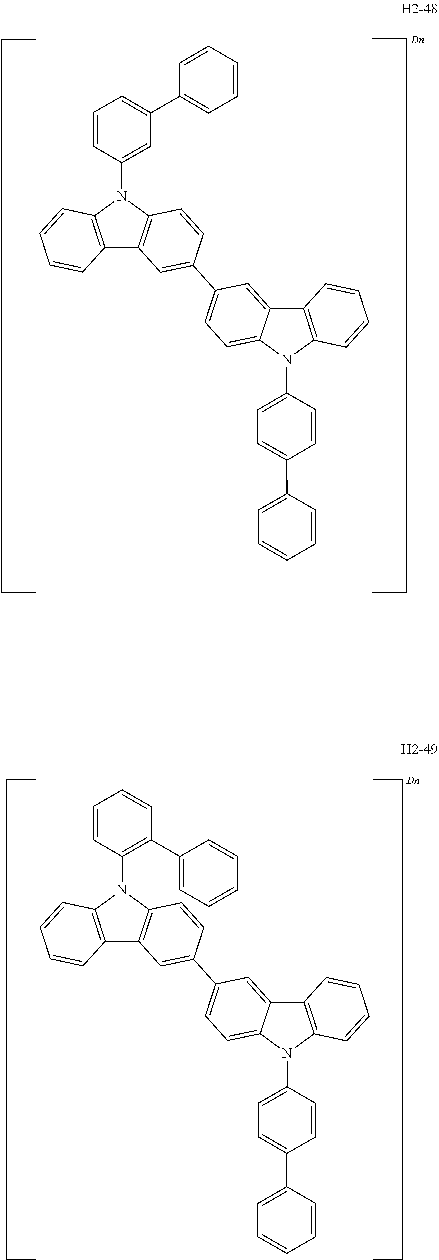

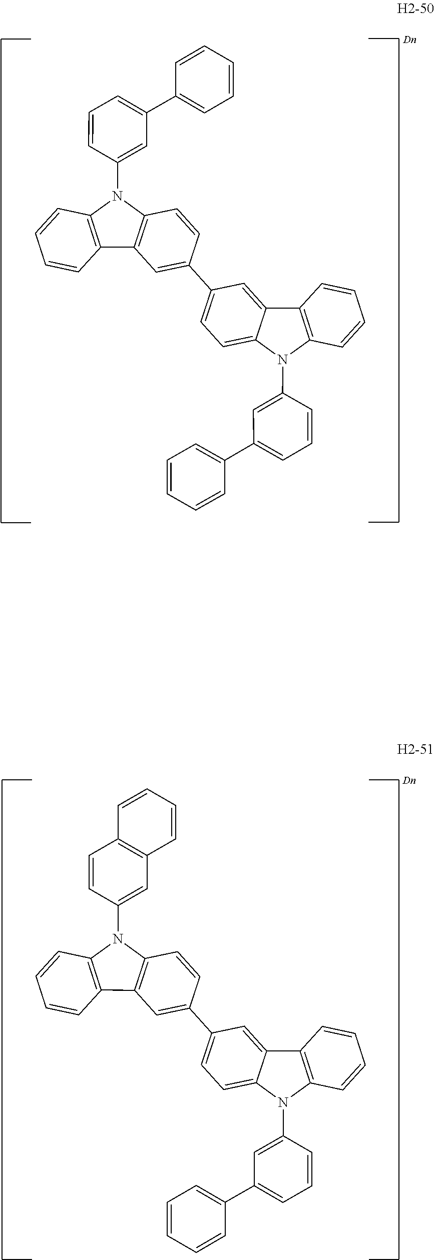

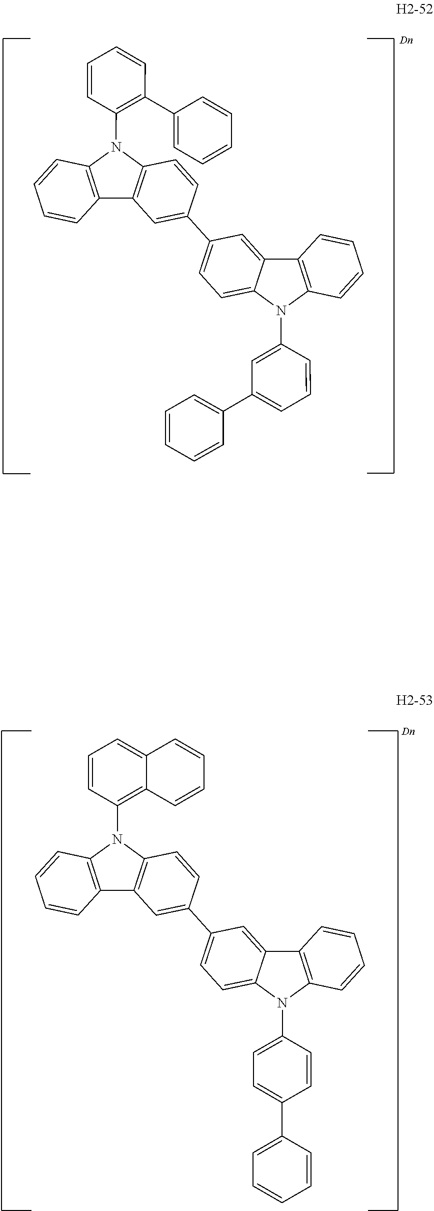

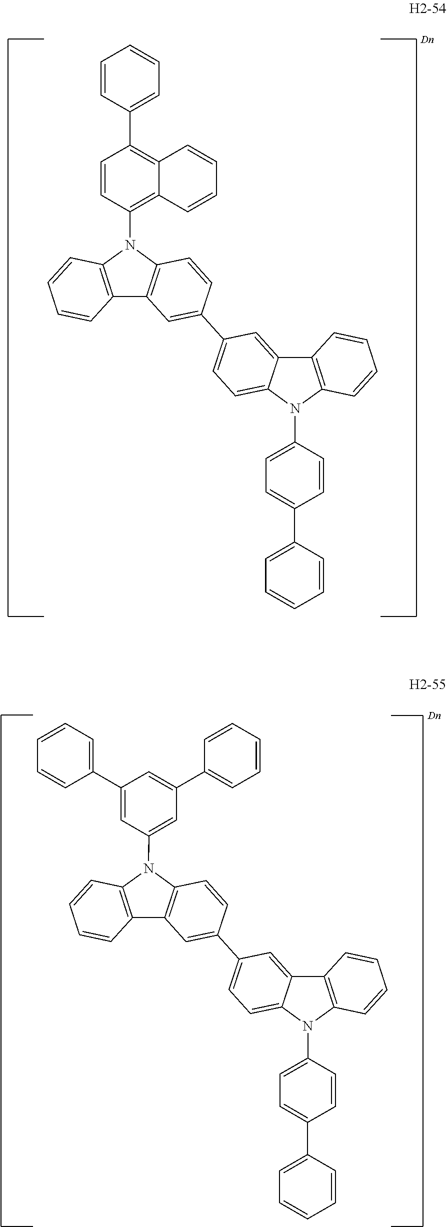

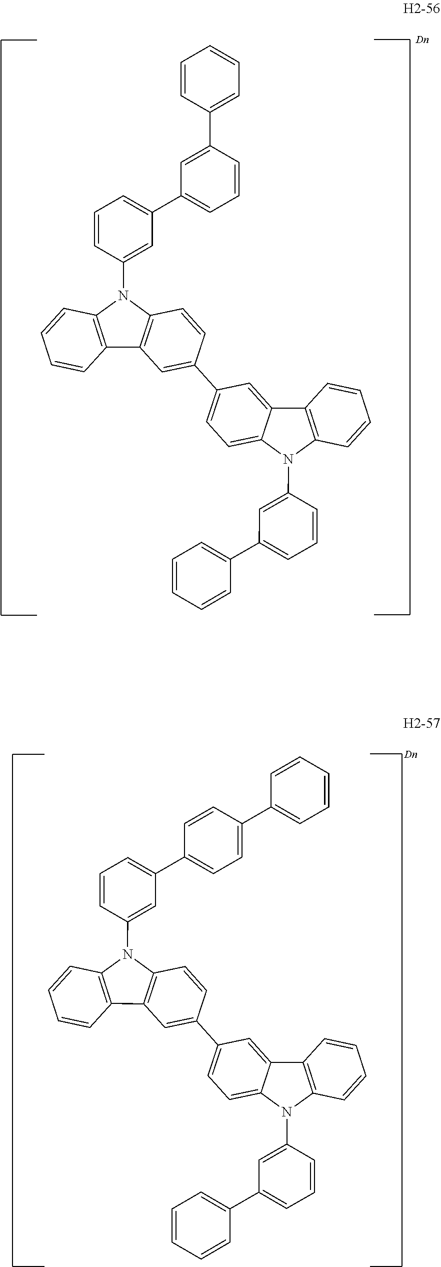

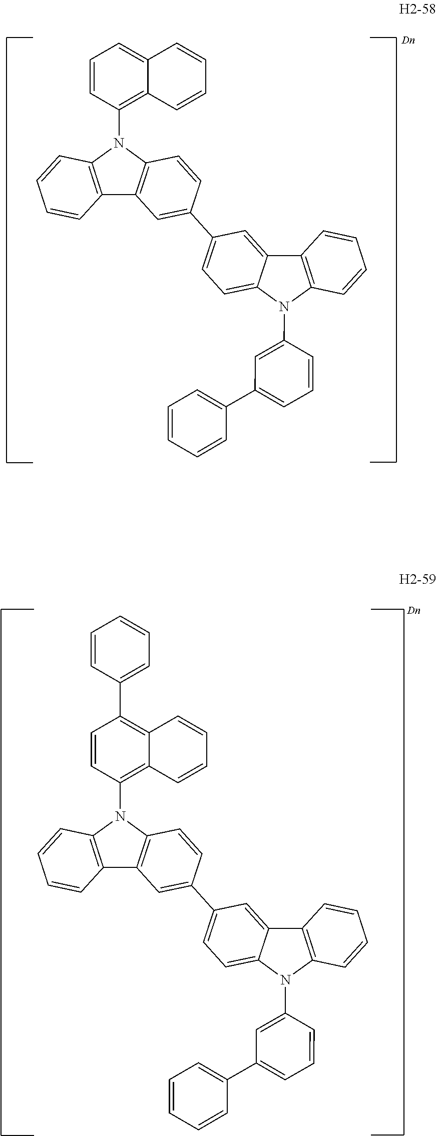

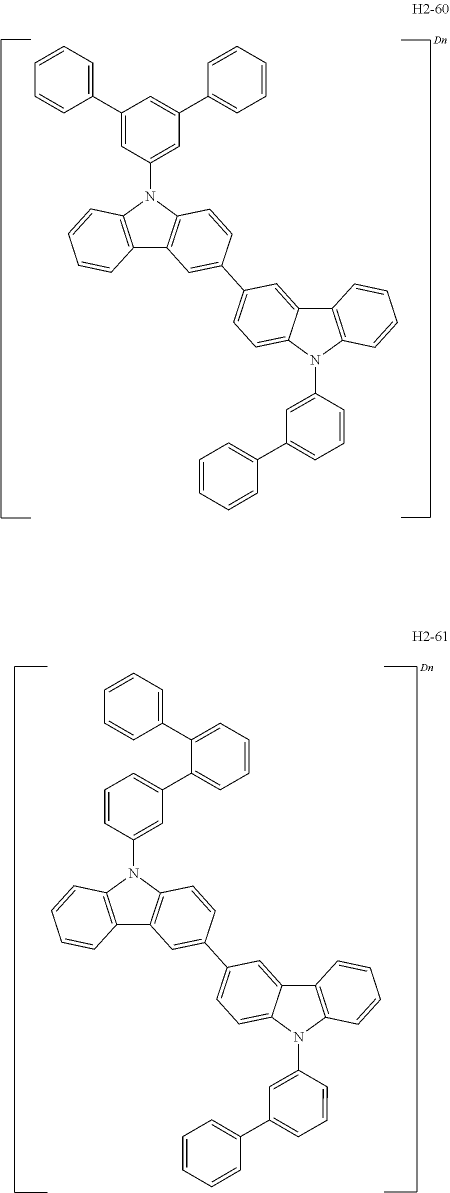

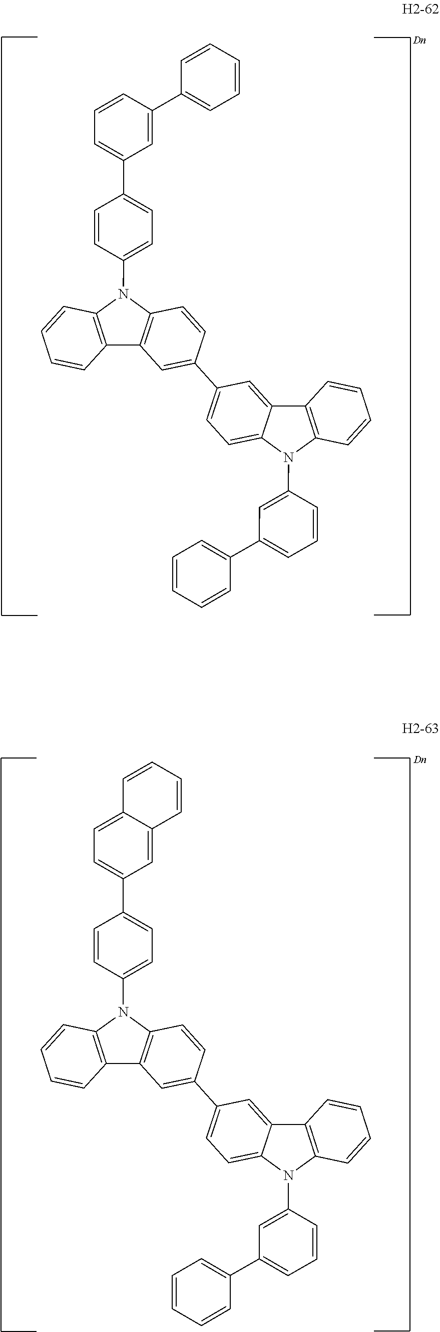

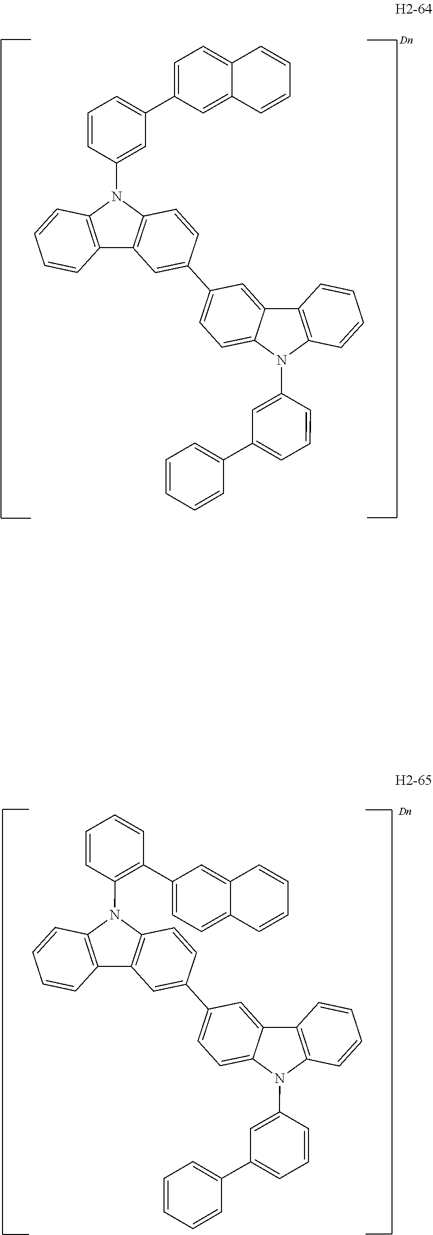

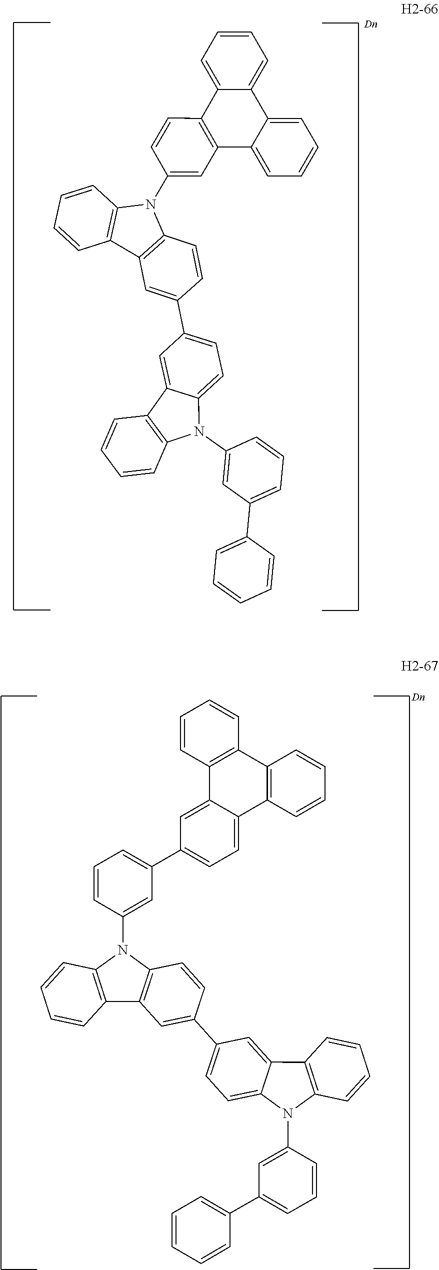

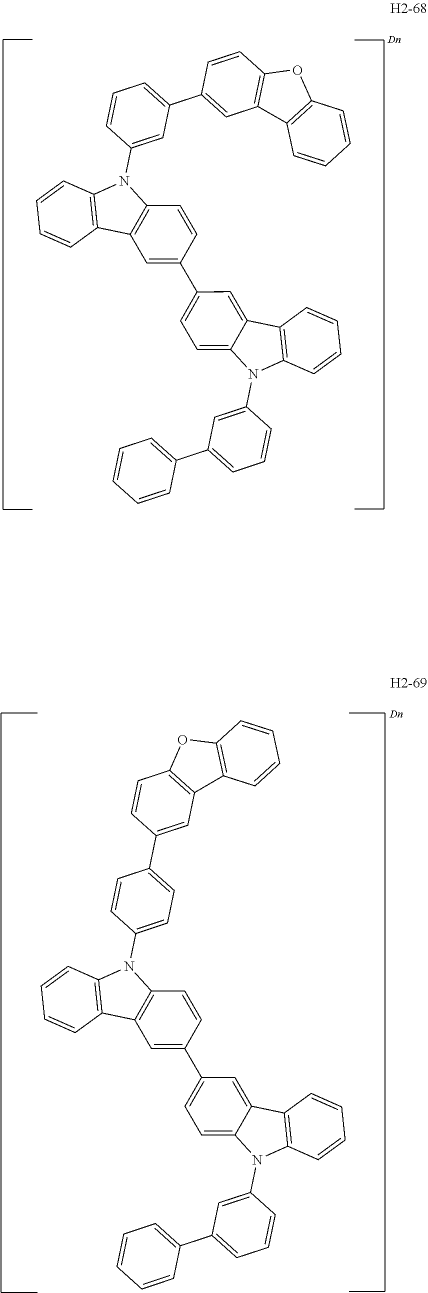

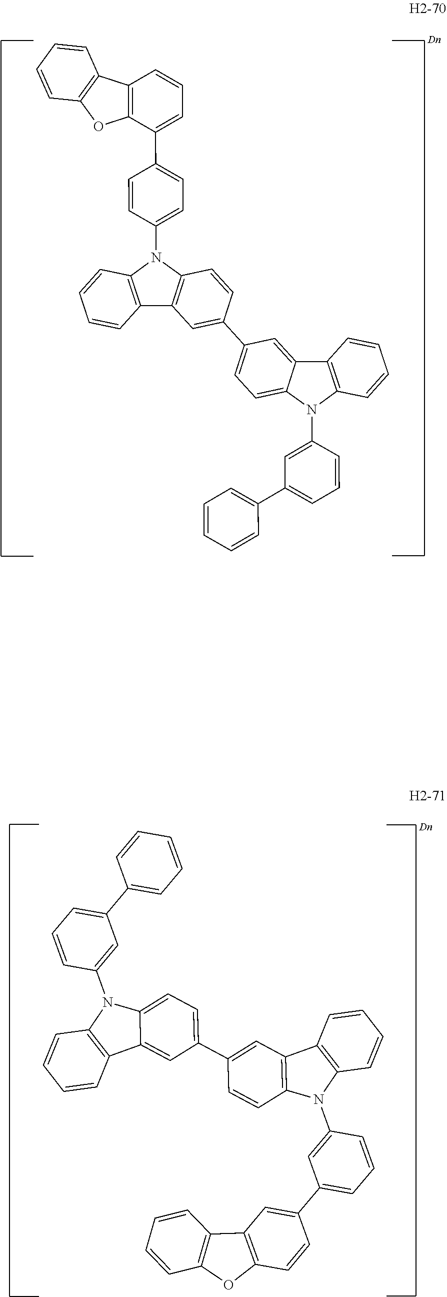

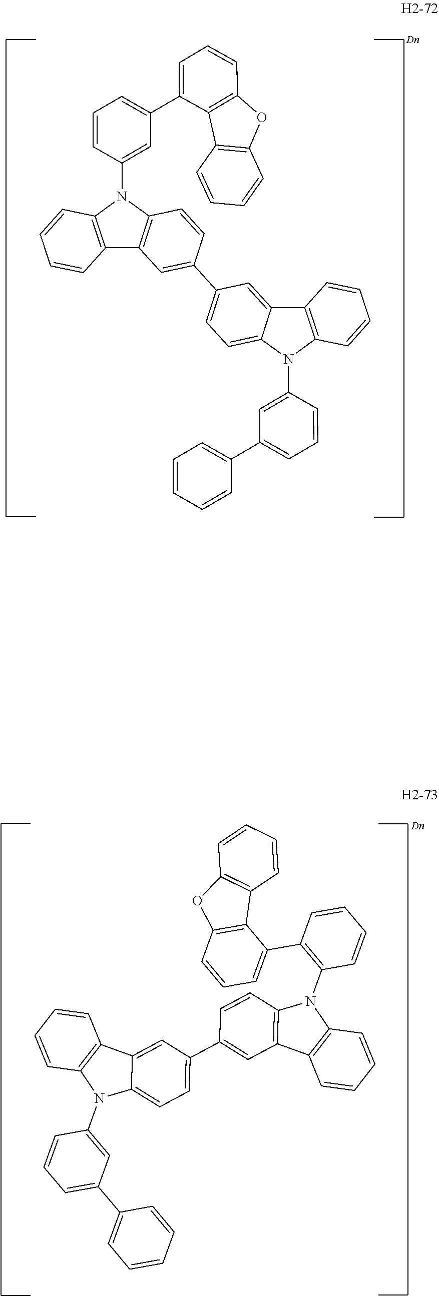

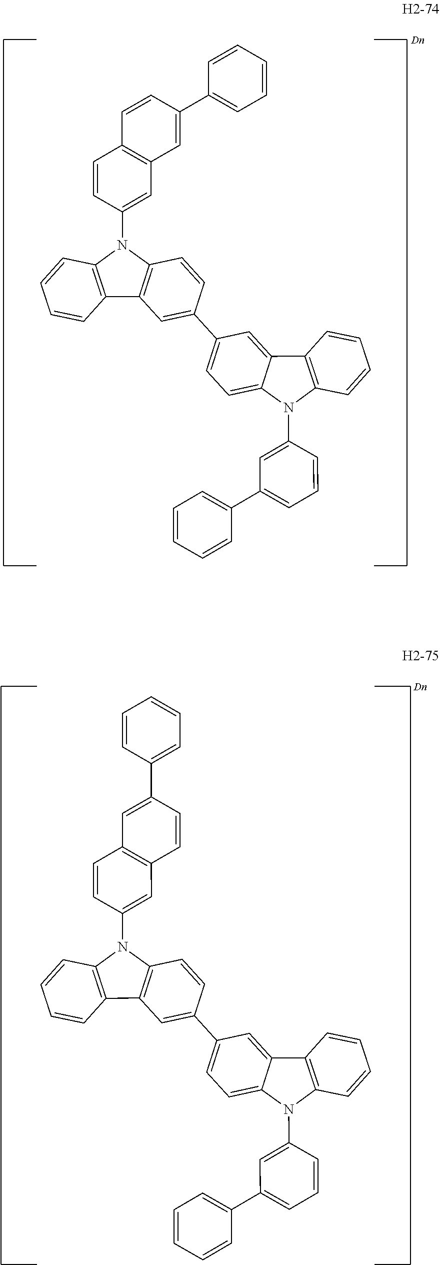

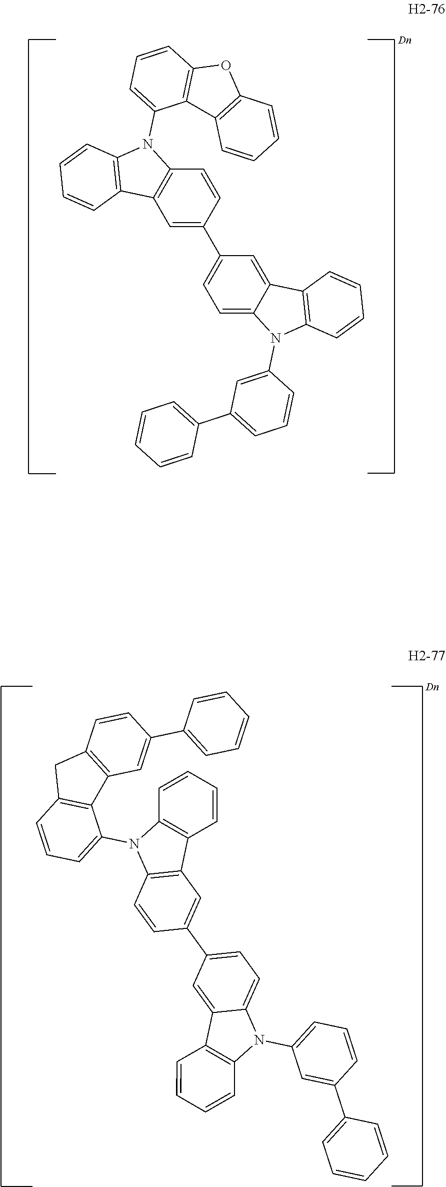

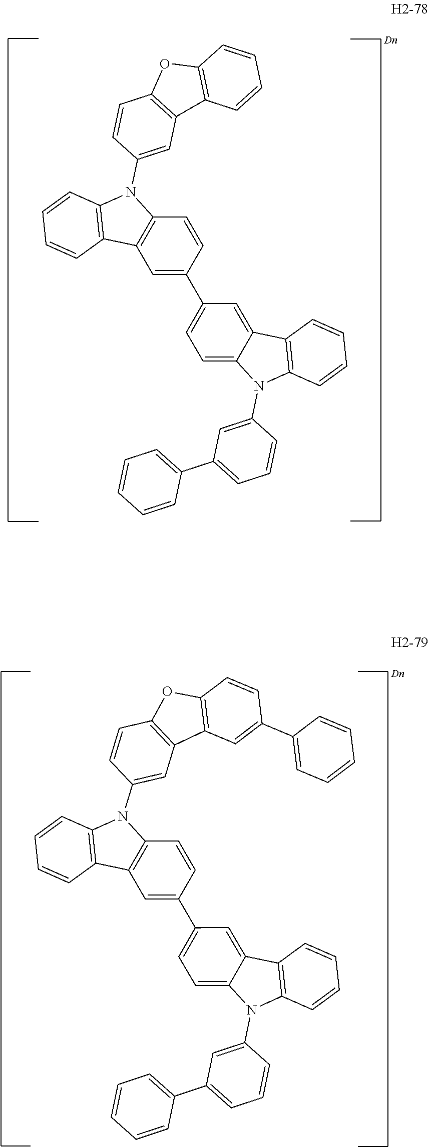

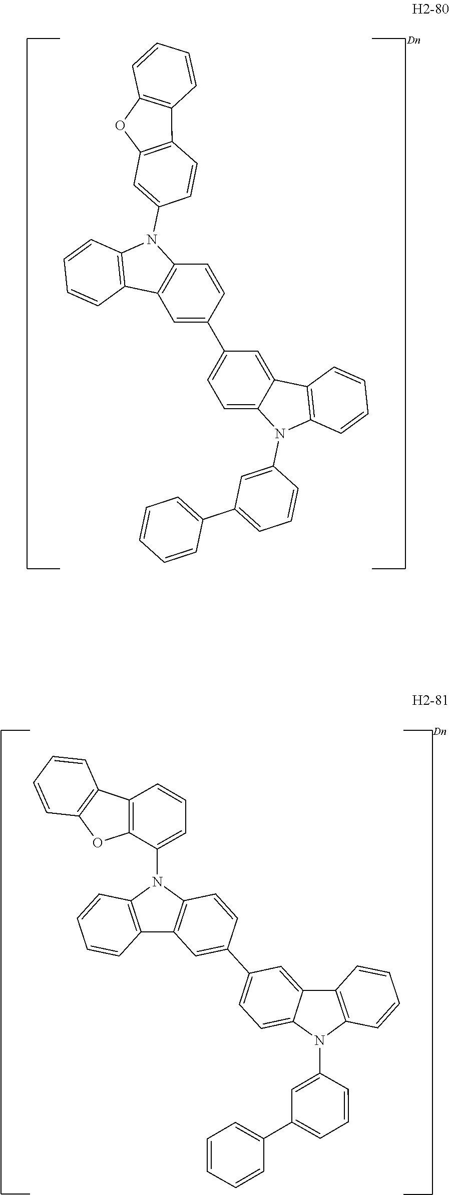

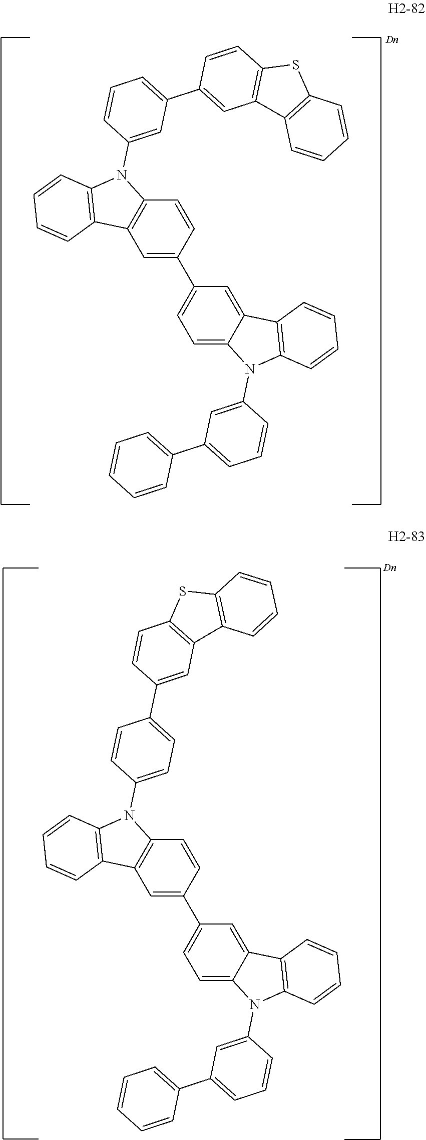

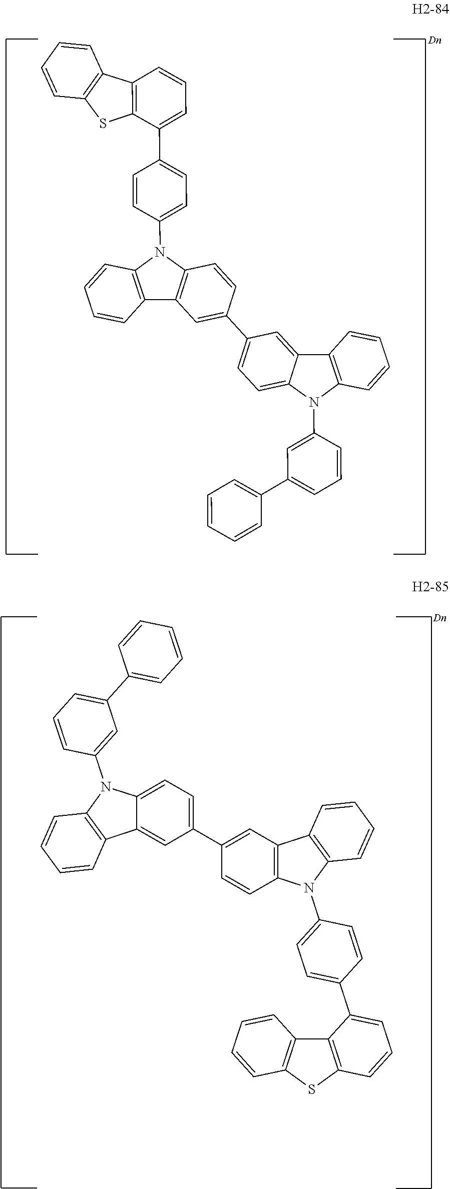

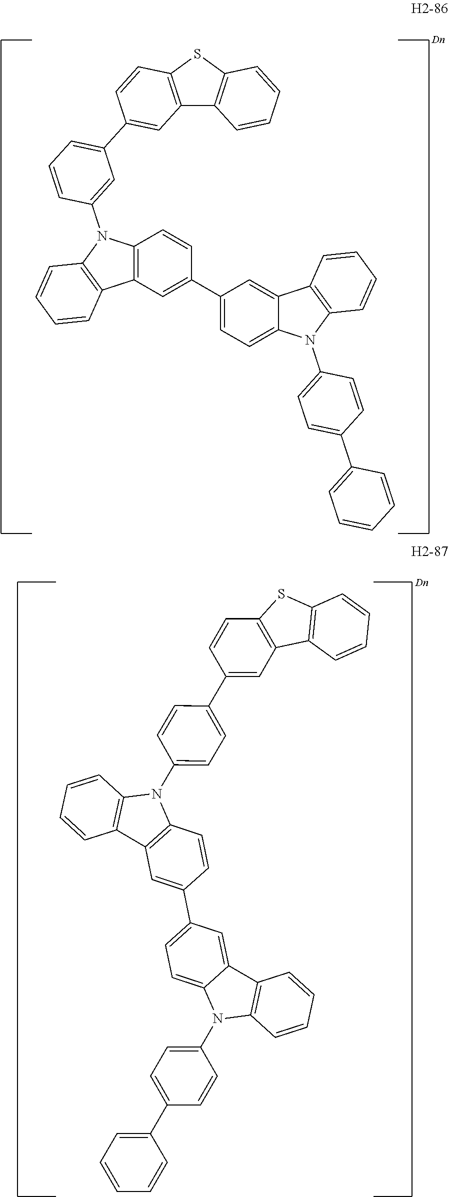

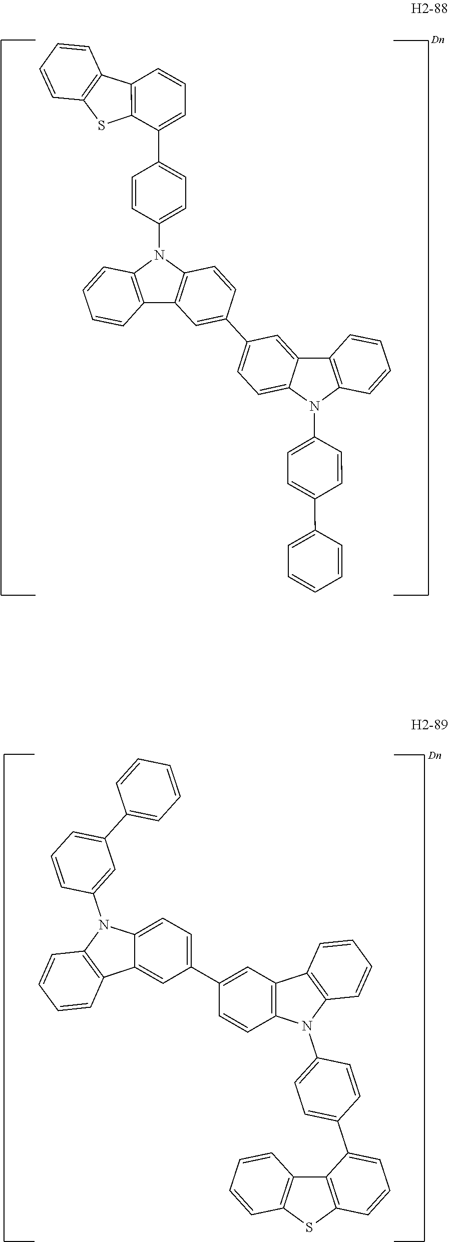

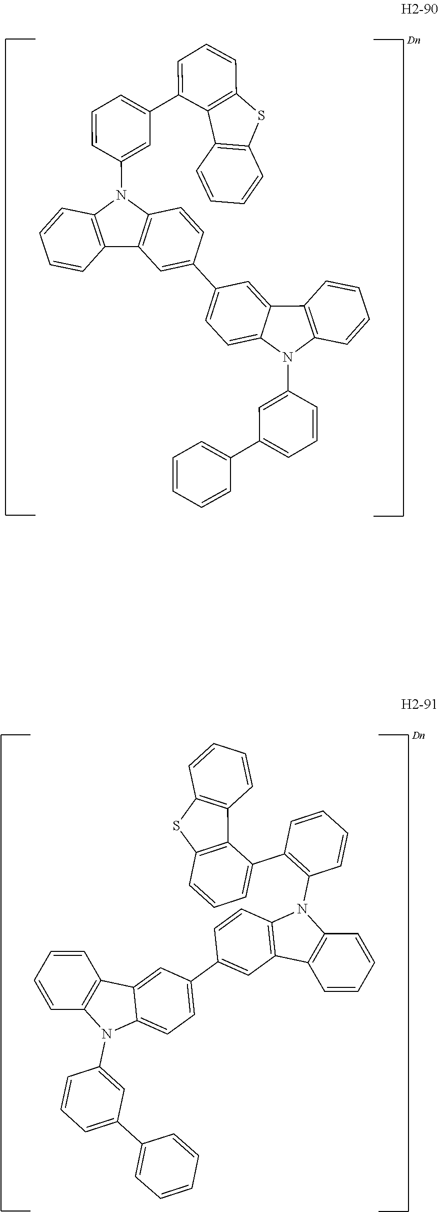

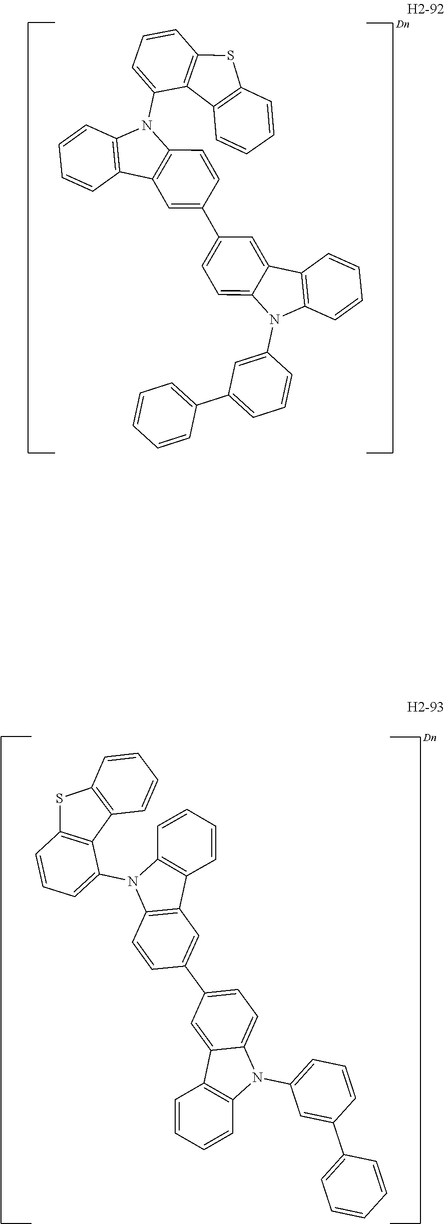

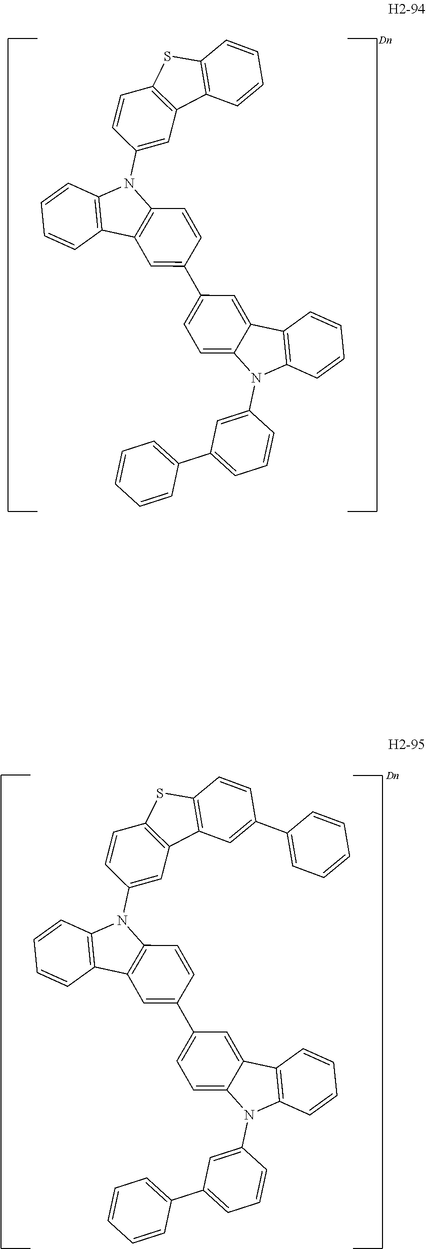

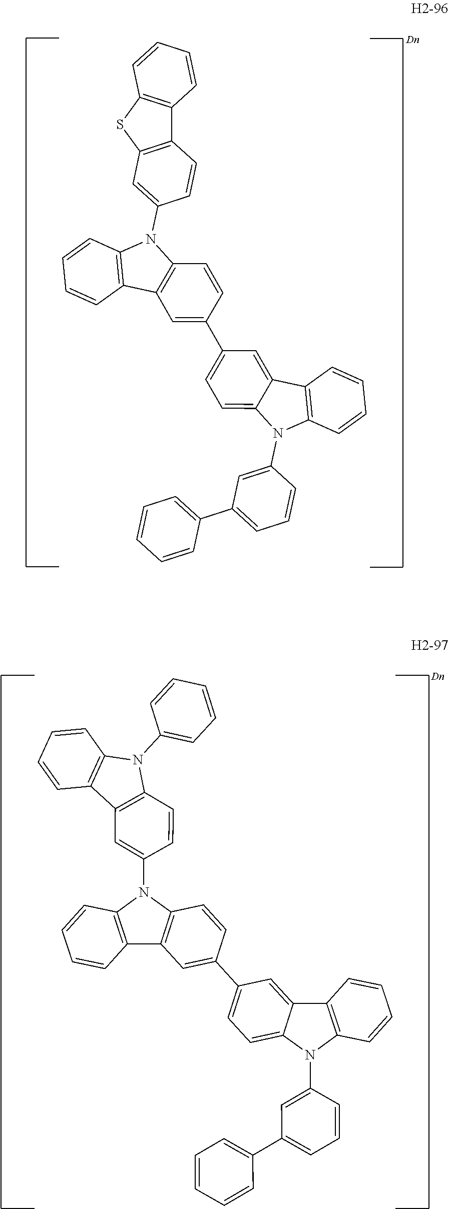

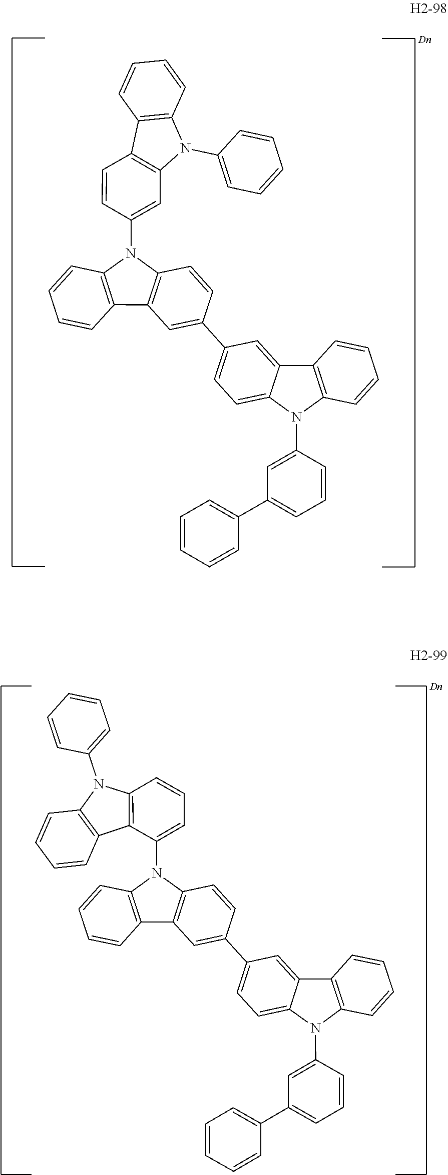

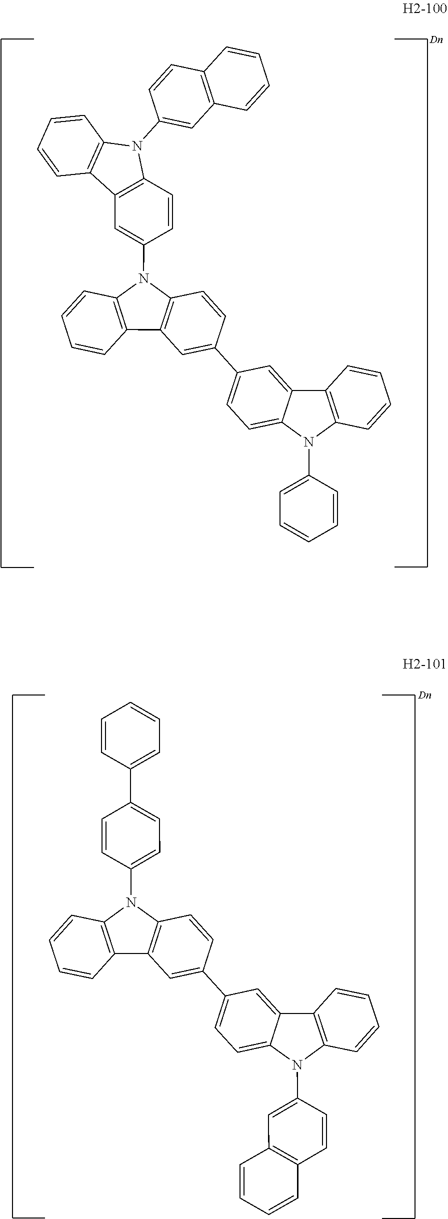

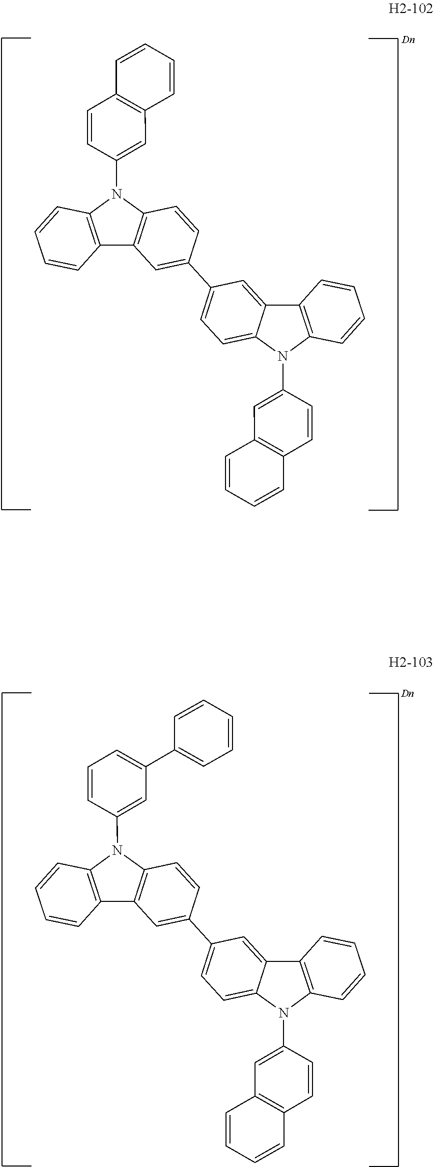

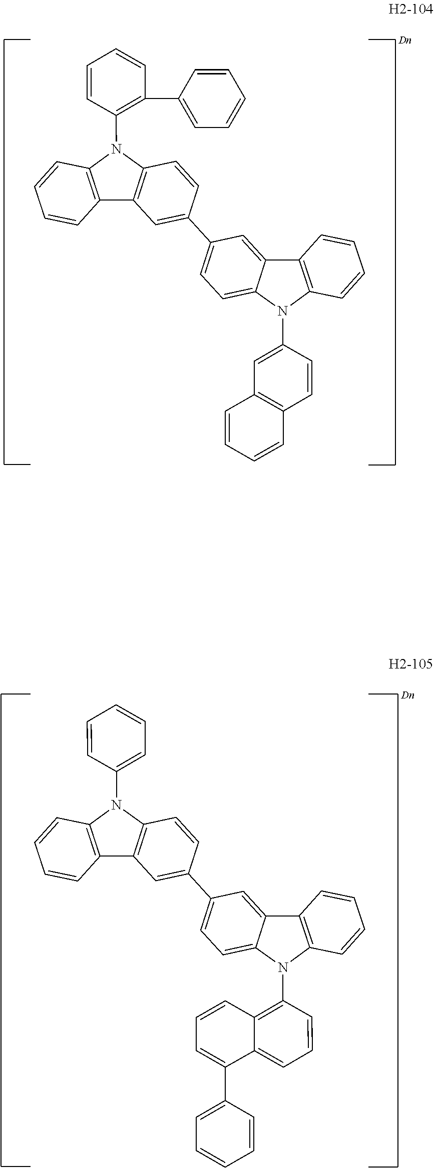

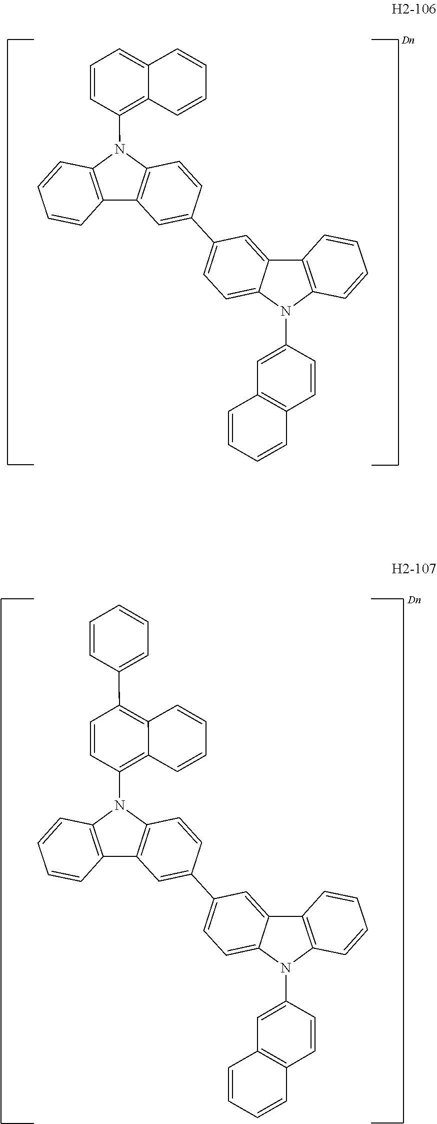

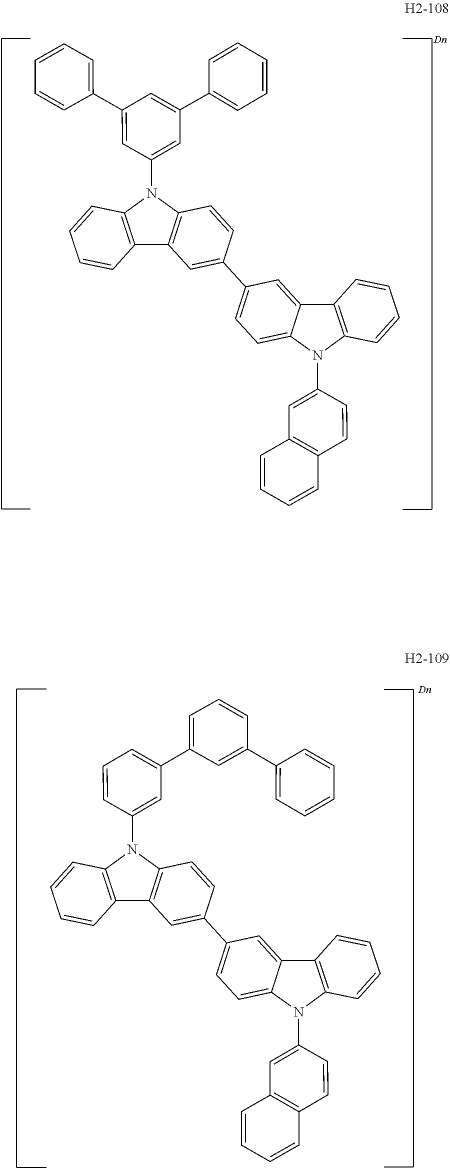

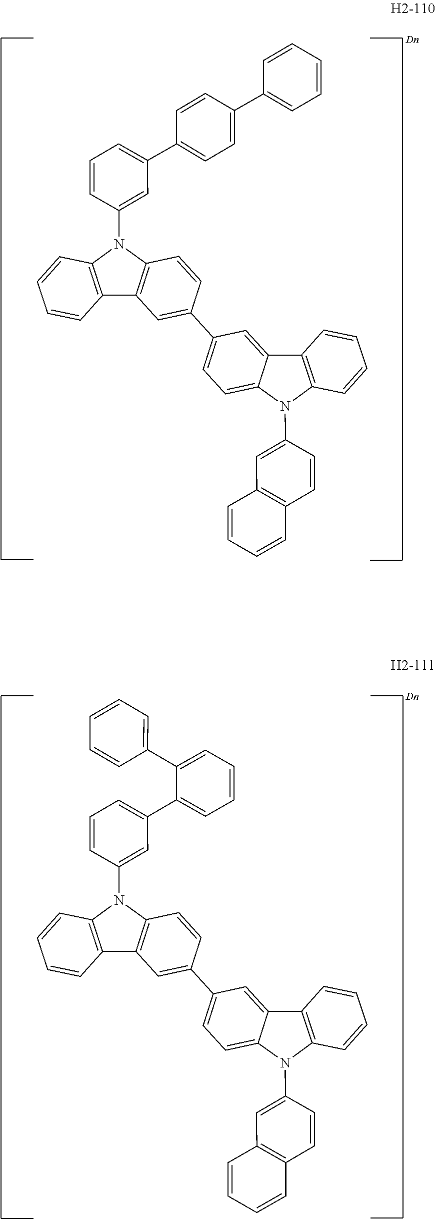

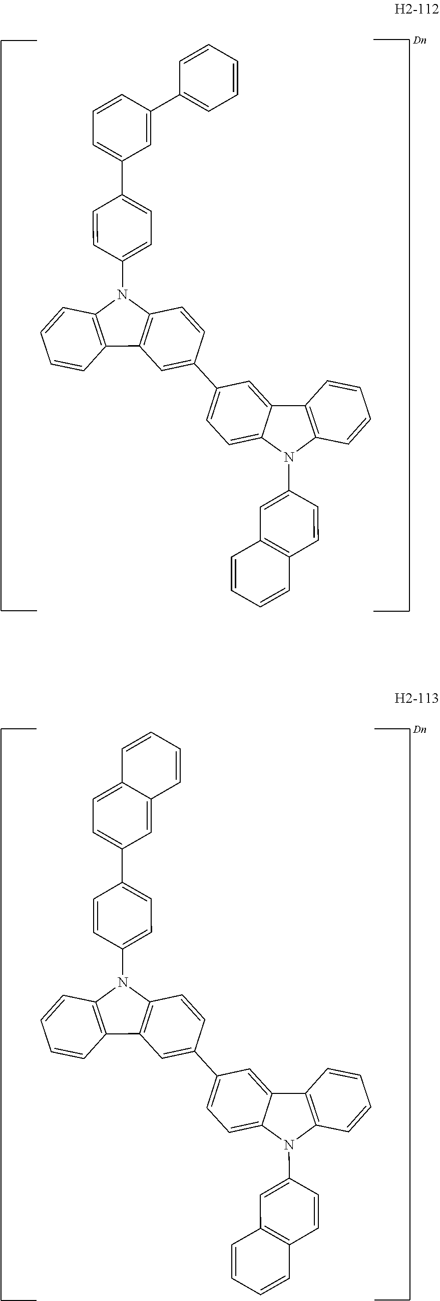

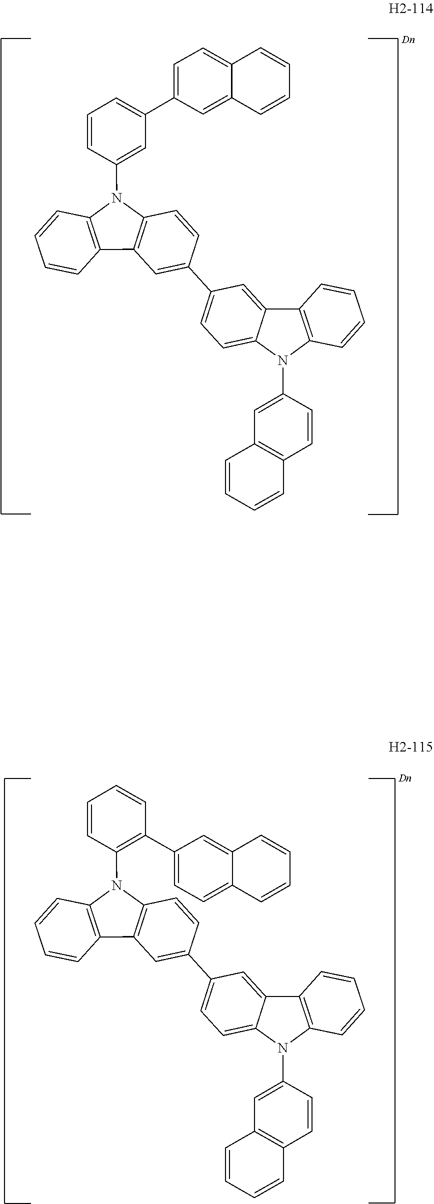

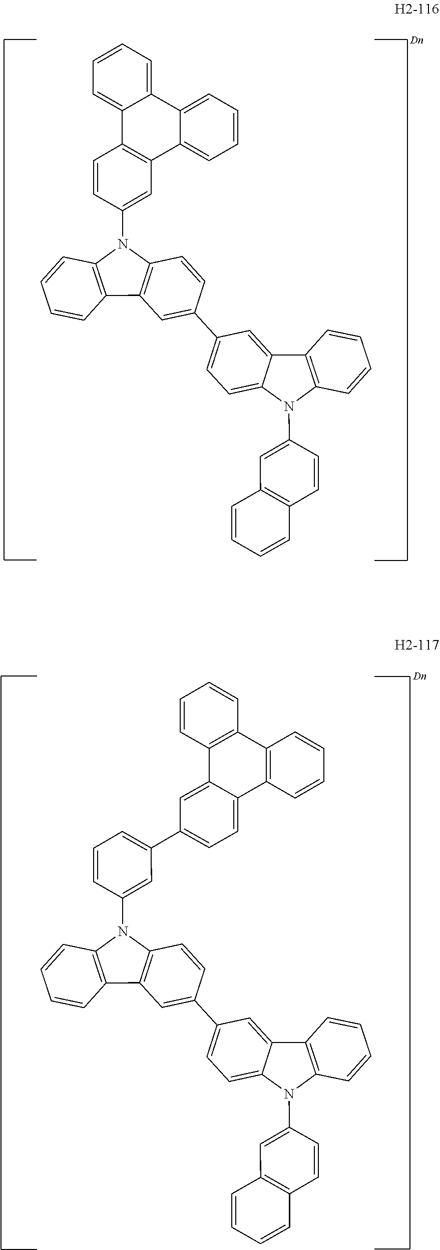

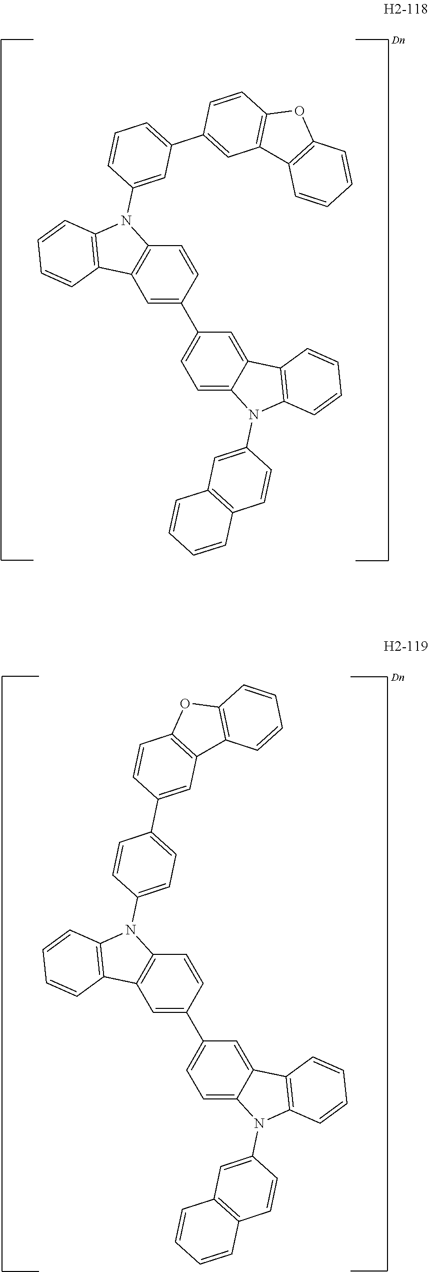

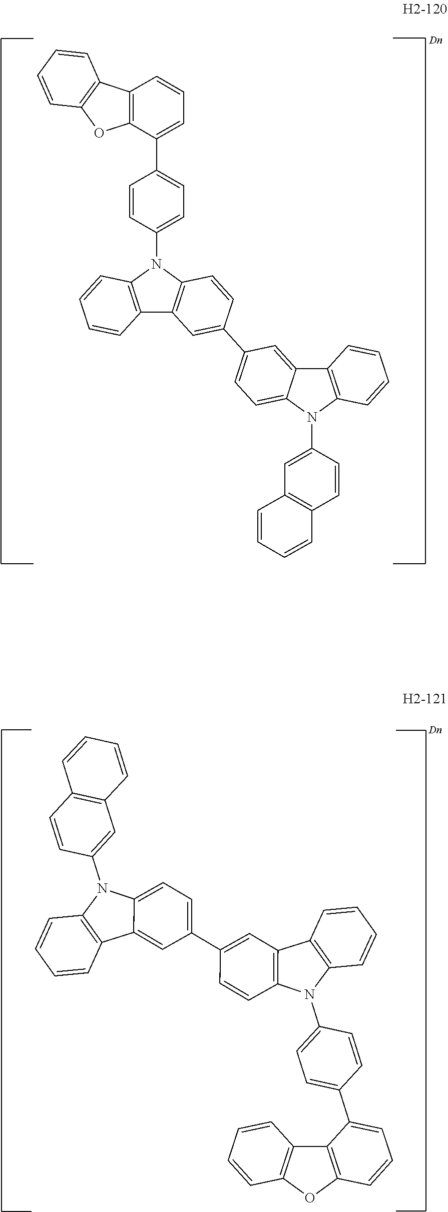

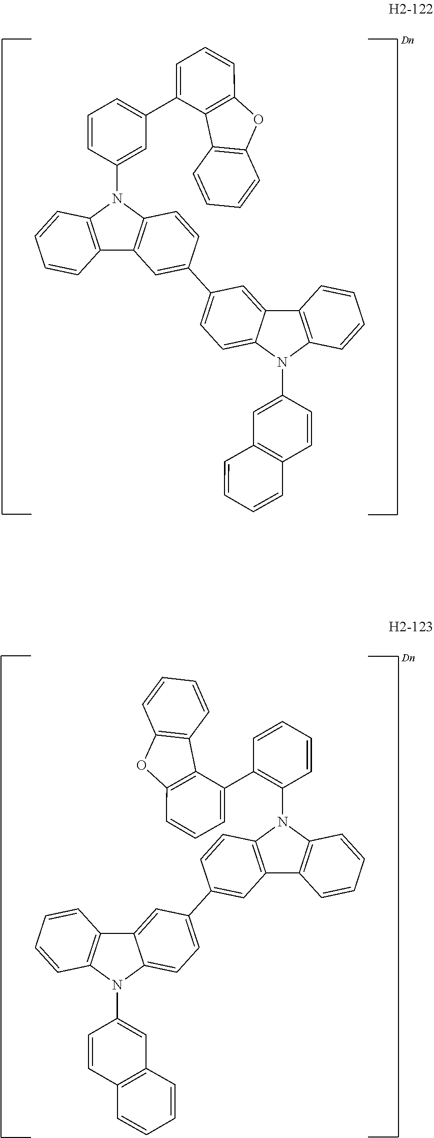

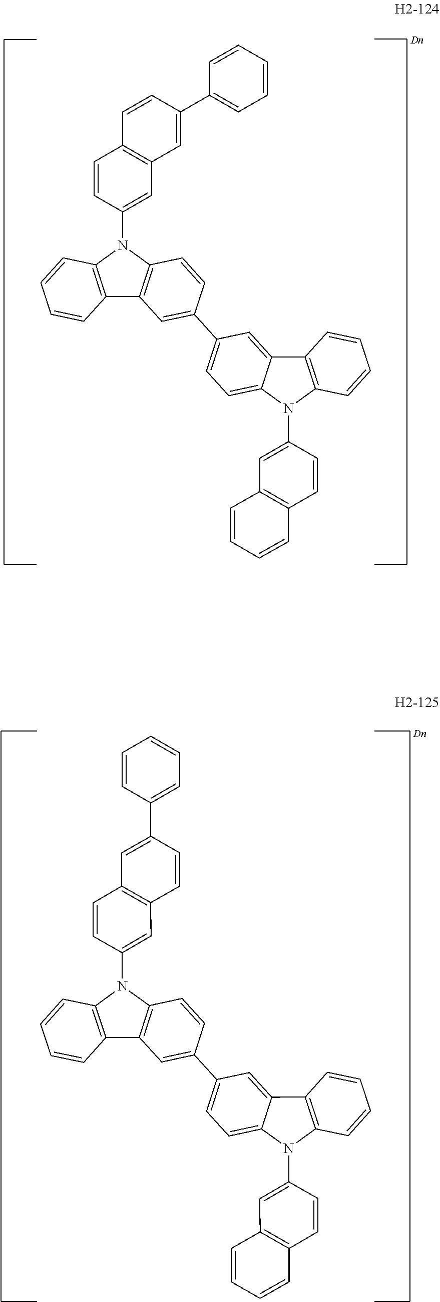

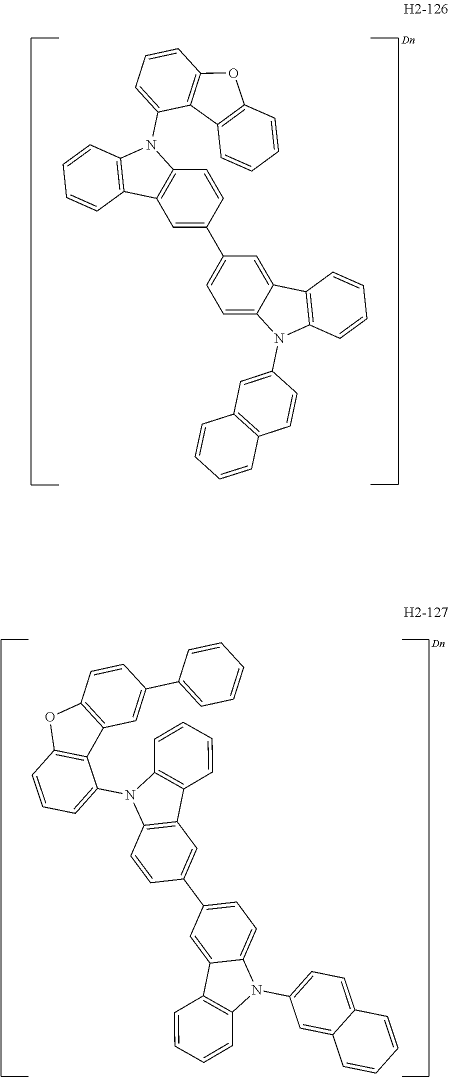

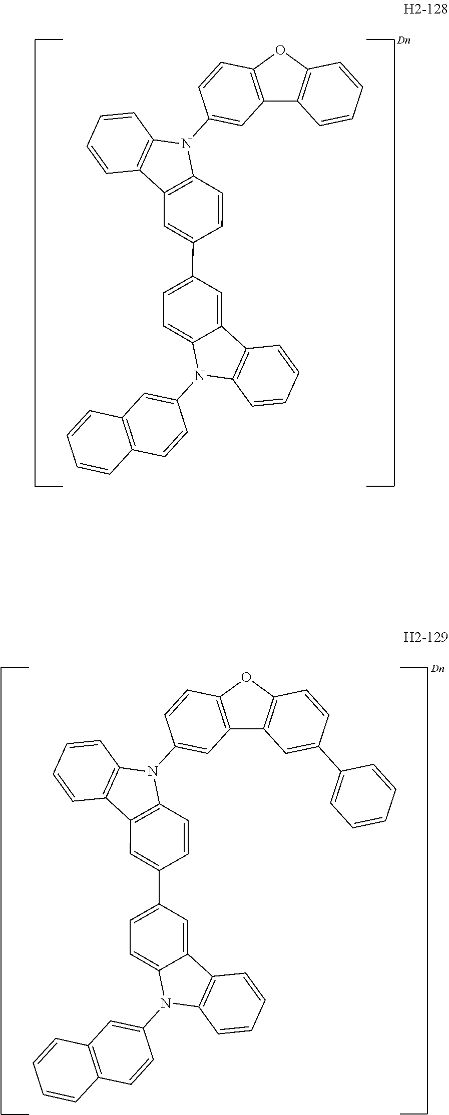

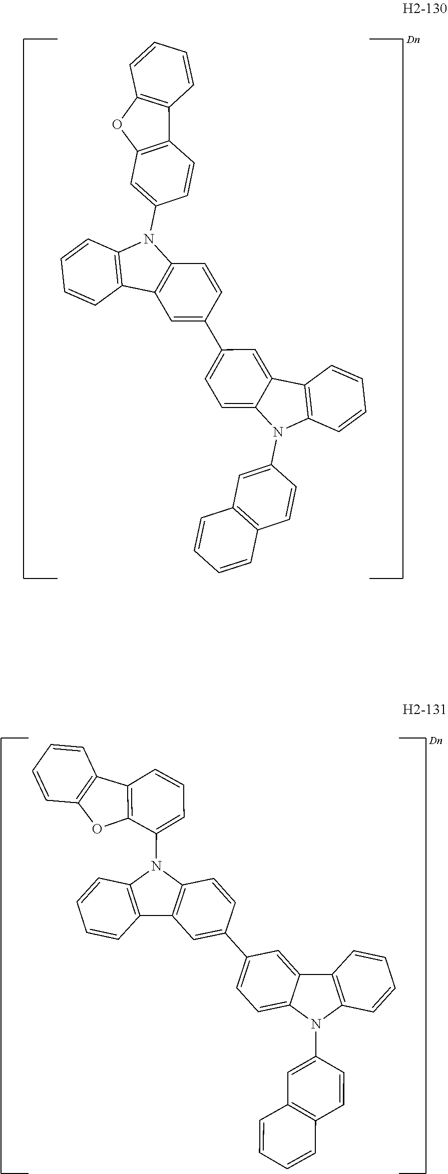

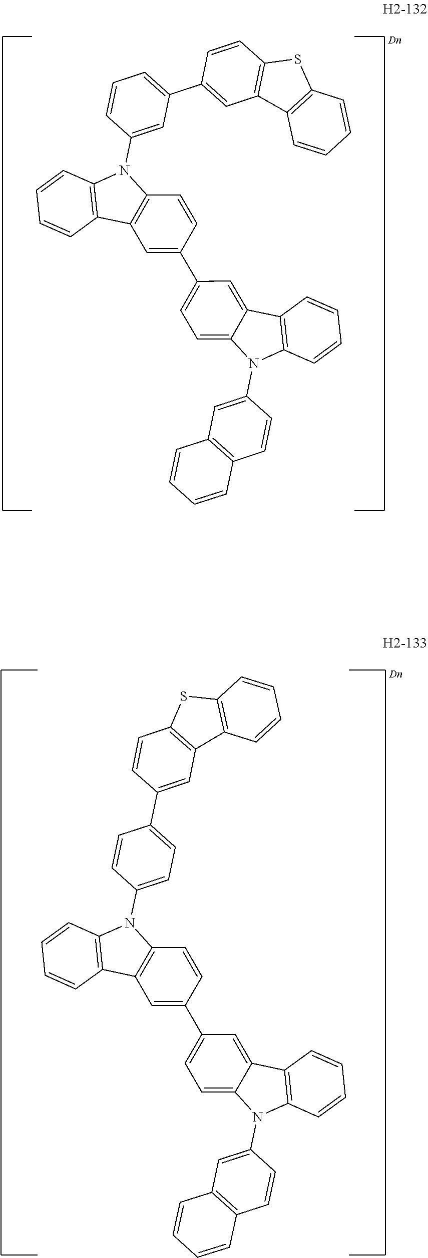

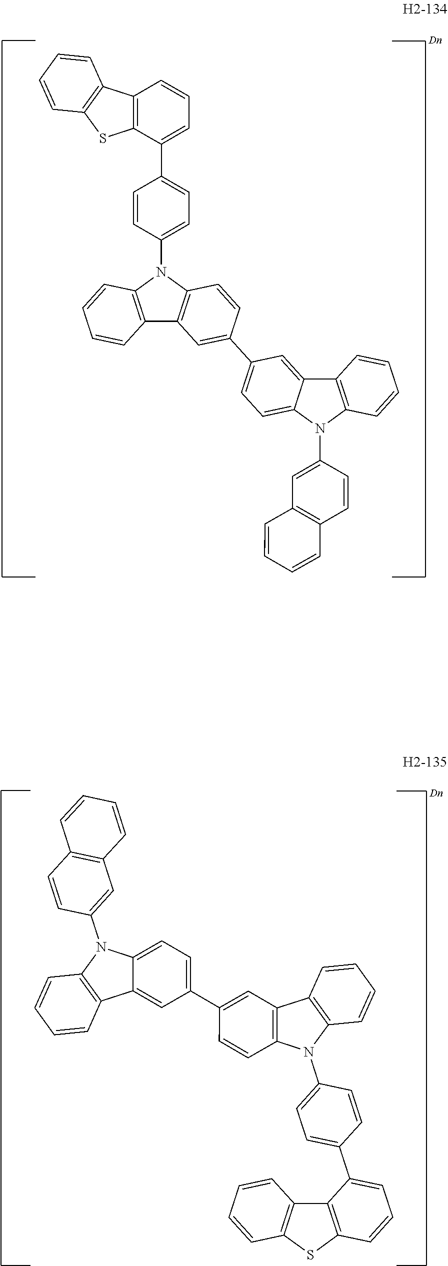

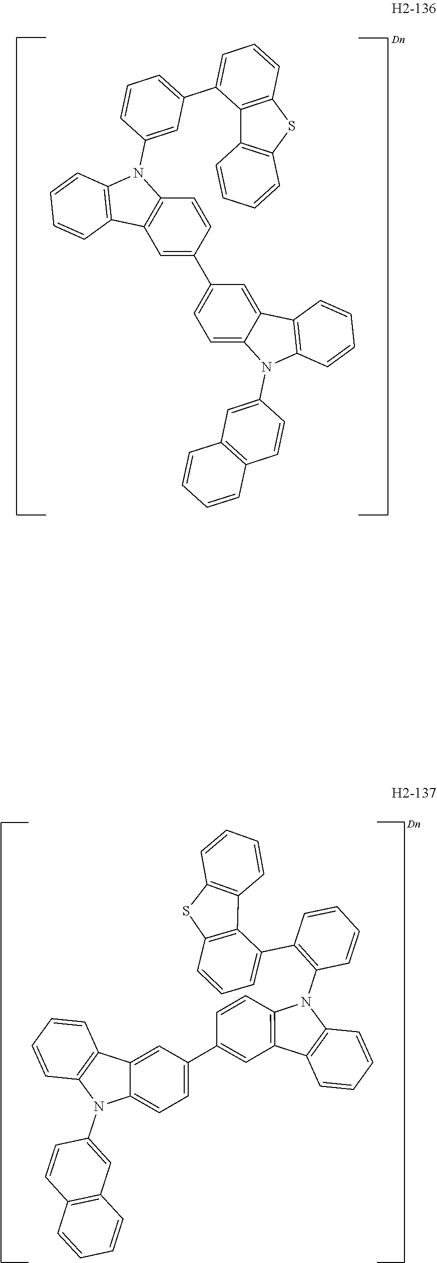

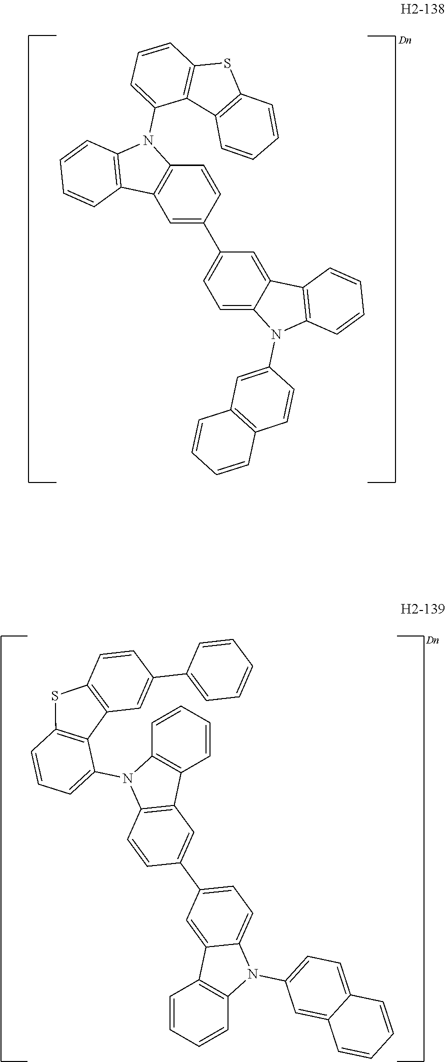

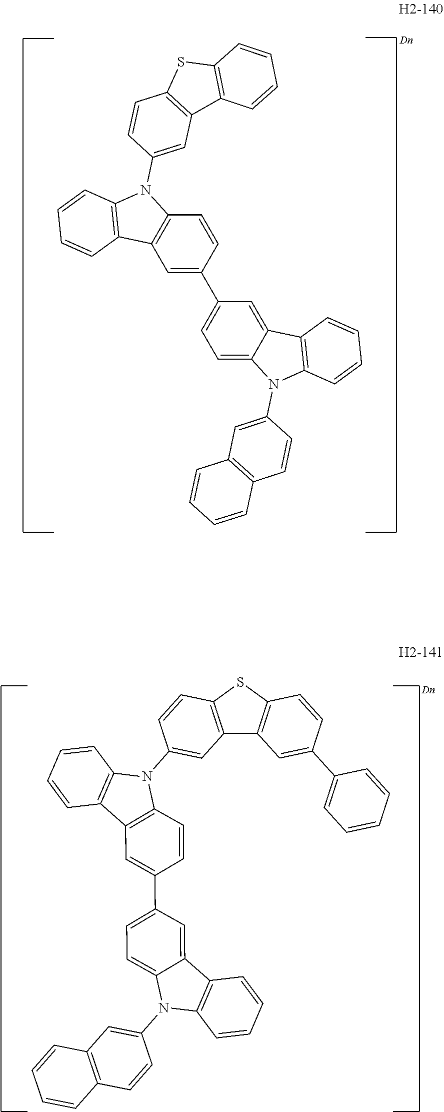

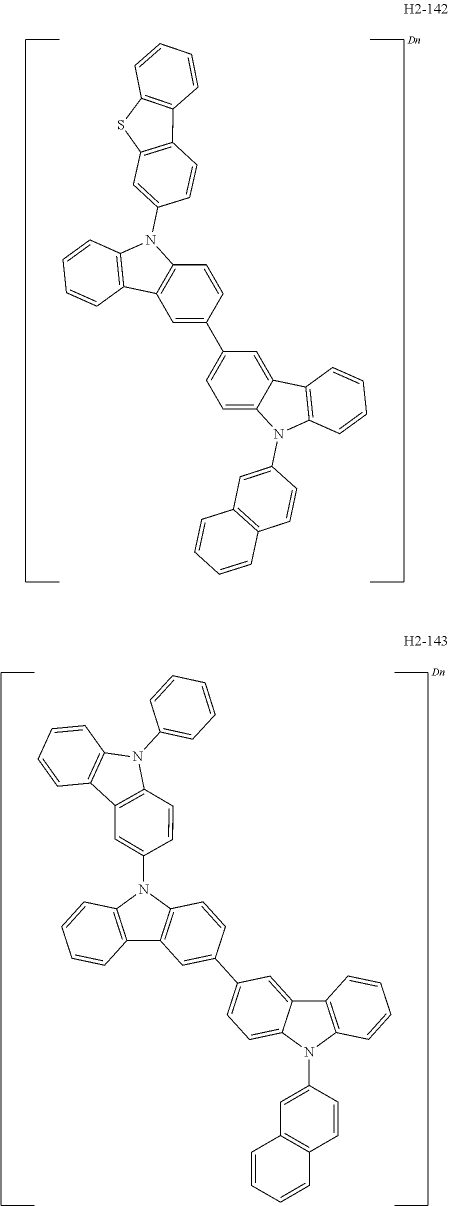

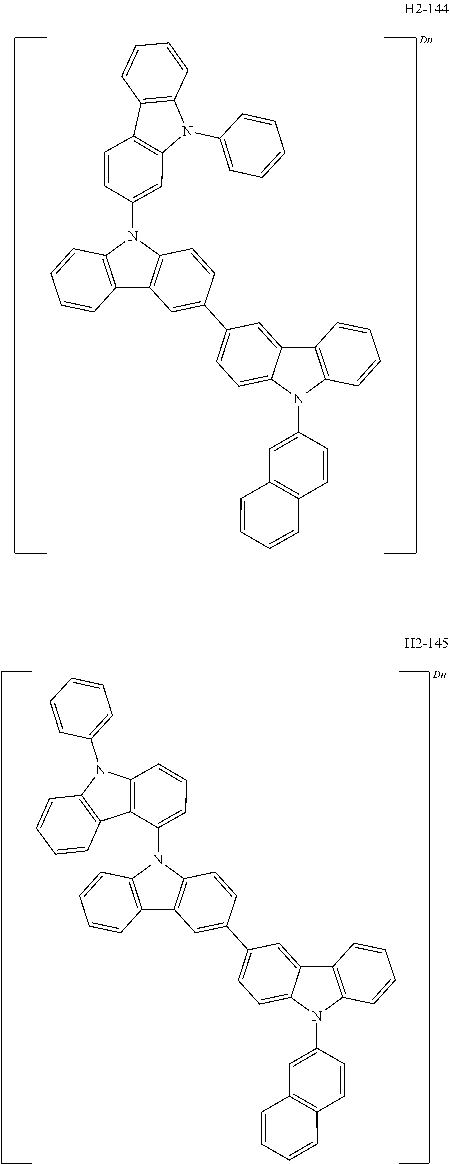

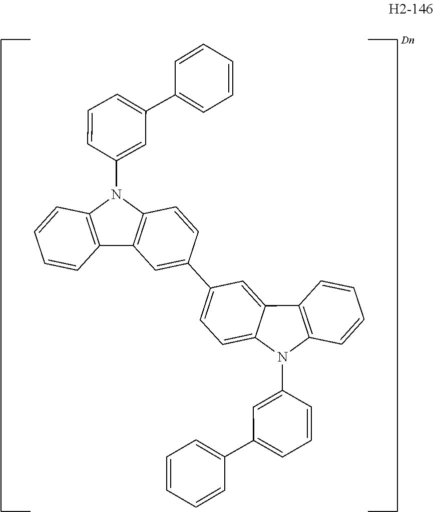

In the compounds above, Dₙ represents that n number of hydrogens are replaced with deuterium; and n is an integer of 1 to 50. According to one embodiment, n is an integer of 4 or more, preferably an integer of 6 or more, more preferably an integer of 8 or more, and even more preferably an integer of 10 or more. When being deuterated to the number of the lower limit or more, the bond dissociation energy related to deuteration may increase to enhance the stability of a compound. When the compound is used in an organic electroluminescent device, the device may exhibit an improved lifetime property.

The combination of at least one of compounds H1-1 to H1-381 and at least one of compounds H2-1 to H2-146 may be used in an organic electroluminescent device.

In addition, the present disclosure provides an organic electroluminescent compound represented by formula 1-A or 1-B, an organic electroluminescent material comprising the compound, and an organic electroluminescent device comprising the compound or the material. The material may consist of the organic electroluminescent compound of the present disclosure alone, or may further comprise conventional materials contained in an organic electroluminescent material. According to one embodiment, the organic electroluminescent compound represented by formula 1-A or 1-B may be comprised in a light-emitting layer as a host material, and may also be comprised in at least one of a hole injection layer, a hole transport layer, a hole auxiliary layer, and a light-emitting auxiliary layer.

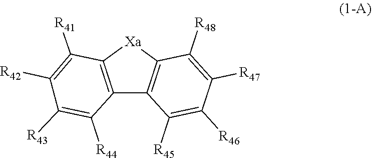

In formula 1-A,

Xₐ represents O or S; and

R₄₁ to R₄₈, each independently, represent hydrogen, deuterium, or a (C6-C18)aryl unsubstituted or substituted with at least one of deuterium, a (C1-C6)alkyl(s) and a (C6-C18)aryl(s), or are represented by the following formula 1-a, with the proviso that at least one of R₄₁ to R₄₈ is represented by the following formula 1-a:

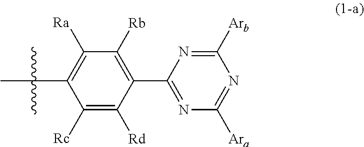

in formula 1-a,

Ra to Rd, each independently, represent hydrogen, deuterium, or a (C6-C18)aryl unsubstituted or substituted with at least one of deuterium, a (C1-C6)alkyl(s) and a (C6-C18)aryl(s); and

Arₐ and Ar_{b}, each independently, represent a phenyl unsubstituted or substituted with deuterium, a substituted or unsubstituted naphthyl, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Ar. and Ar_{b} comprises a substituted or unsubstituted carbazolyl.

For example, in formula 1-a, Arₐ and Ar_{b}, each independently, may be a phenyl unsubstituted or substituted with at least one of deuterium, a dibenzofuranyl(s) and a carbazolyl(s); a biphenyl unsubstituted or substituted with at least one deuterium; a terphenyl unsubstituted or substituted with at least one deuterium; a dibenzofuranyl unsubstituted or substituted with at least one deuterium; a dibenzothiophenyl unsubstituted or substituted with at least one deuterium; or a carbazolyl unsubstituted or substituted with at least one of deuterium and a phenyl(s), etc.

According to one embodiment, in formula 1-a, at least one of Ar. and Ar_{b} may be represented by the following formula 1-bi or 1-b2.

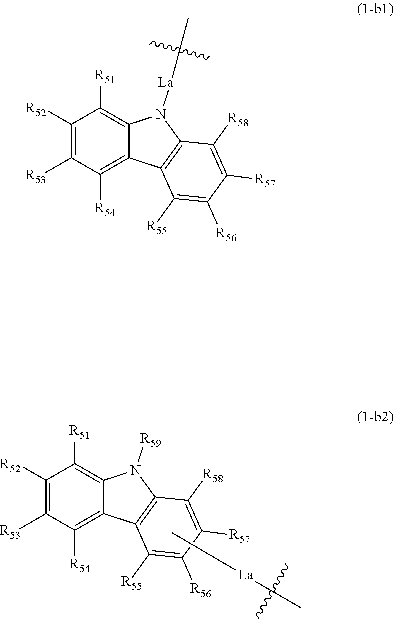

In formulas 1-b1 and 1-b2,

La represents a single bond, a phenylene unsubstituted or substituted with deuterium, a naphthylene unsubstituted or substituted with deuterium, a biphenylene unsubstituted or substituted with deuterium, or a terphenylene unsubstituted or substituted with deuterium; and

R₅₁ to R₅₉, each independently, represent hydrogen, deuterium, a phenyl unsubstituted or substituted with deuterium, a naphthyl unsubstituted or substituted with deuterium, a biphenyl unsubstituted or substituted with deuterium, a terphenyl unsubstituted or substituted with deuterium, or a combination thereof.

The compound represented by formula 1-A may be at least one selected from the group consisting of compounds H1-271 to H1-279, H1-281 to H1-289, H1-291 to H1-299, H1-320 to H1-322, and H1-329 to H1-331, but is not limited thereto.

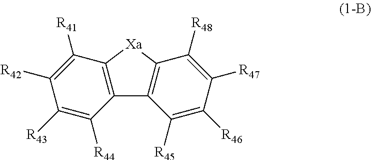

In formula 1-B,

Xu represent O or S;

R₄₁ to R₄₈, each independently, represent hydrogen; deuterium; a (C6-C18)aryl unsubstituted or substituted with at least one of deuterium, a (C1-C6)alkyl(s) and a (C6-C18)aryl(s); or -L_{b}-HAr_{b}, with the proviso that at least one of R₄₁ to R₄₈ represents -L_{b}-HAr_{b};

L_{b} represents a naphthylene unsubstituted or substituted with deuterium; and

HAr_{b} is represented by the following formula 1-b:

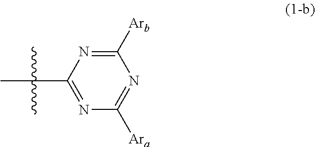

in formula 1-b,

Arₐ and Ar_{b}, each independently, represent a phenyl unsubstituted or substituted with deuterium, a naphthyl unsubstituted or substituted with deuterium, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Arₐ and Ar_{b} comprises a carbazolyl unsubstituted or substituted with at least one of deuterium and a (C6-C18)aryl(s), a dibenzofuranyl unsubstituted or substituted with deuterium, or a dibenzothiophenyl unsubstituted or substituted with deuterium.

According to one embodiment, R₄₁ to R₄₈, each independently, represent hydrogen, deuterium, or -L_{b}-HAr_{b}. According to another embodiment, any one of R₄₁ to R₄₈ represents -L_{b}-HAr_{b}.

According to one embodiment, in formula 1-b, Arₐ and Ar_{b}, each independently, may be a phenyl, a naphthyl, a naphthylphenyl, a phenylnaphthyl, a biphenyl, a terphenyl, a dibenzofuranyl unsubstituted or substituted with a phenyl(s), a dibenzothiophenyl unsubstituted or substituted with a phenyl(s), or a carbazolyl unsubstituted or substituted with a phenyl(s), which may be further substituted with at least one deuterium. According to another embodiment, at least one of Arₐ and Ar_{b} comprises a carbazolyl unsubstituted or substituted with at least one of deuterium and a phenyl(s), a dibenzofuranyl unsubstituted or substituted with deuterium, or a dibenzothiophenyl unsubstituted or substituted with deuterium.

The compound represented by formula 1-B may be at least one selected from the group consisting of compounds H1-342 to H1-381, but is not limited thereto.

The compounds represented by formulas 1, 1-A, and 1-B according to the present disclosure may be produced by synthetic methods known to one skilled in the art, and for example, by referring to the following reaction scheme 1, but is not limited thereto.

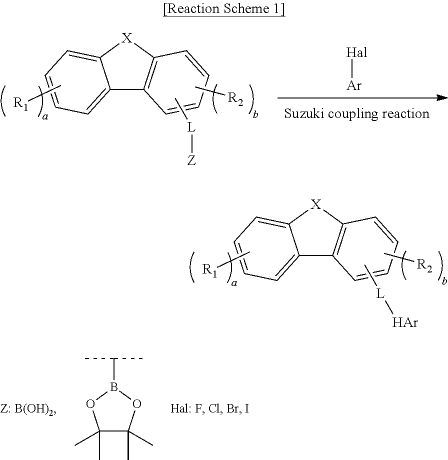

In reaction scheme 1, X, HAr, L, R₁, R₂, a, and b are as defined in formula 1.

The compound represented by formula 2 according to the present disclosure may be produced by a synthetic method known to one skilled in the art, and for example, by referring to the following reaction scheme 2, but is not limited thereto.

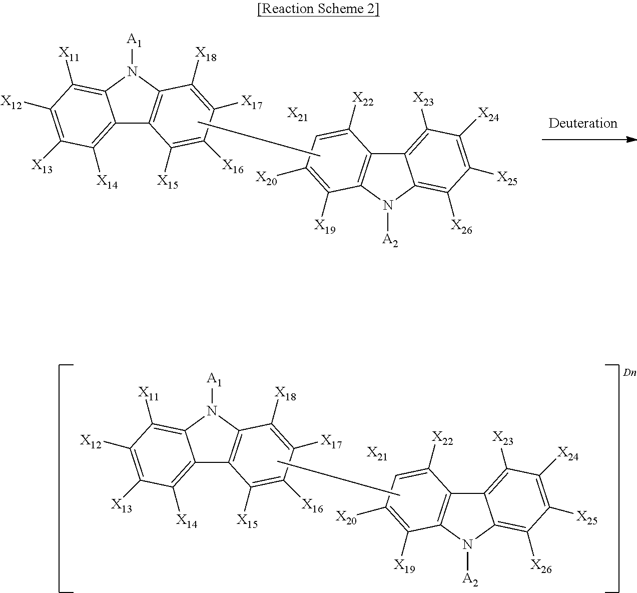

In reaction scheme 2, A₁, A₂, X₁₁ to X₂₆, and n are as defined in formula 2, and Dn represents that n number of hydrogens are replaced with deuterium.

Although illustrative synthesis examples of the compounds represented by formulas 1, 1-A, 1-B, and 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formulas 1, 1-A, 1-B and 2 above, but are not specified in the specific synthesis examples, are bonded.

In addition, the deuterated compounds of formula 1, 1-A, 1-B, and 2 may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride, a trifluoromethanesulfonic acid, or a trifluoromethanesulfonic acid-D. In addition, the degree of deuteration can be controlled by changing the reaction conditions such as the reaction temperature. For example, the number of deuterium in formulas 1, 1-A, 1-B, and 2 can be controlled by adjusting the reaction temperature and time, the equivalent of the acid, etc.

The present disclosure provides an organic electroluminescent device comprising an anode, a cathode, and at least one light-emitting layer between the anode and cathode in which the at least one light-emitting layer comprises a plurality of host materials according to the present disclosure. The first host material and the second host material according to the present disclosure may be comprised in one light-emitting layer, or may be respectively comprised in different light-emitting layers. The ratio of the compound represented by formula 1 and the compound represented by formula 2 in the plurality of host materials is about 1:99 to about 99:1, preferably about 10:90 to about 90:10, more preferably about 30:70 to about 70:30. In addition, the compound represented by formula 1 and the compound represented by formula 2 in a desired ratio may be combined by mixing them in a shaker, by dissolving them in a glass tube by heat, or by dissolving them in a solvent, etc.

According to one embodiment, the doping concentration of the dopant compound with respect to the host compound in the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated indium complex compounds.

The dopant comprised in the organic electroluminescent device of the present disclosure may be a compound represented by the following formula 101, but is not limited thereto.

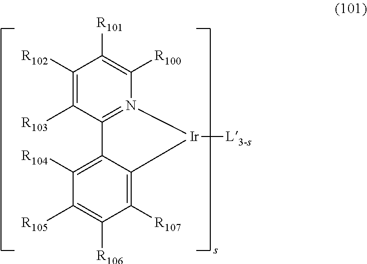

In formula 101,

L′ is selected from the following structures 1 to 3:

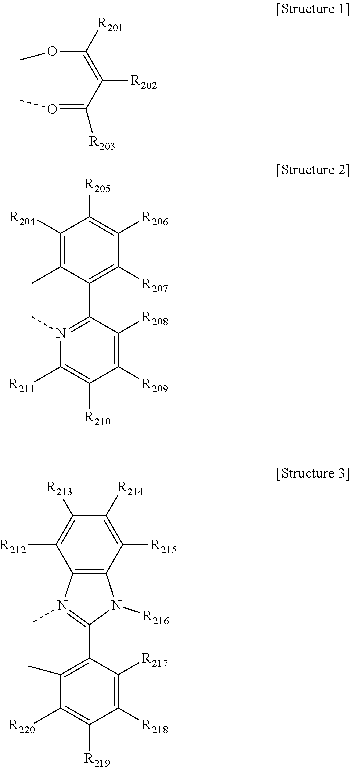

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and

s represents an integer of 1 to 3.

The specific examples of the dopant compound are as follows, but are not limited thereto.

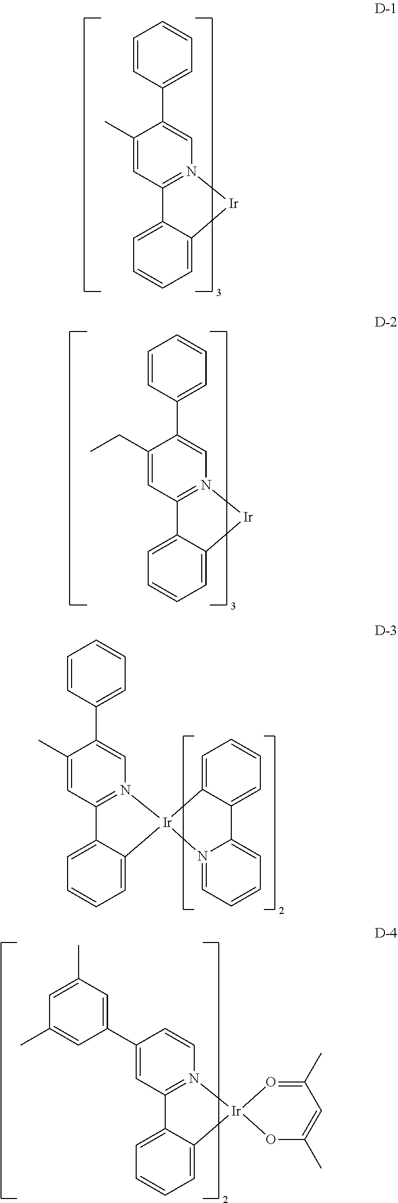

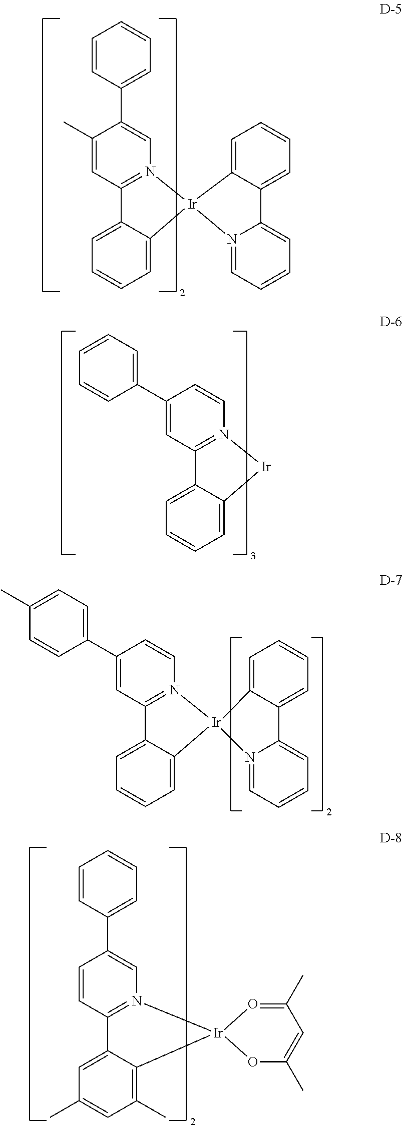

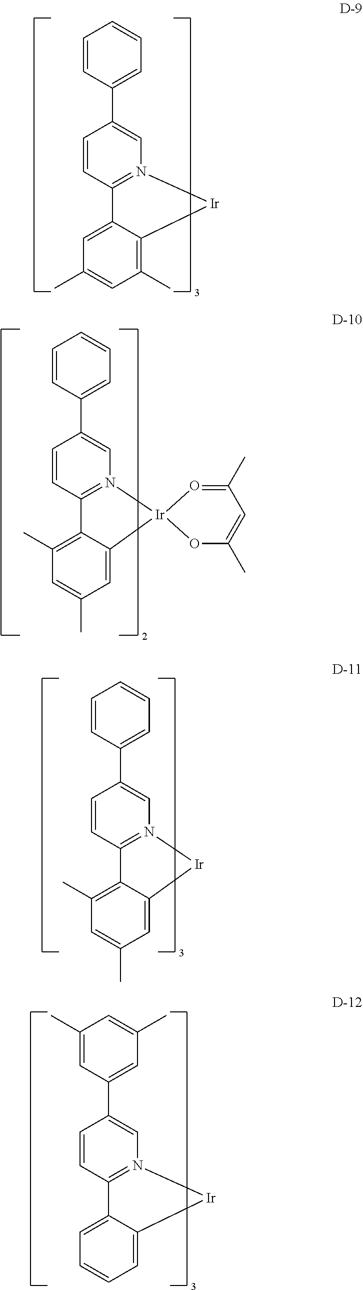

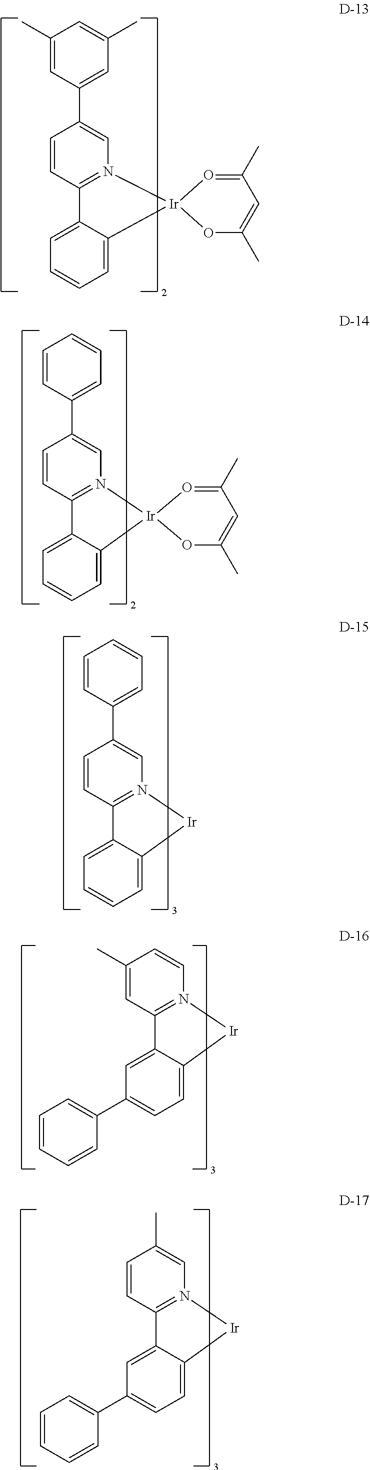

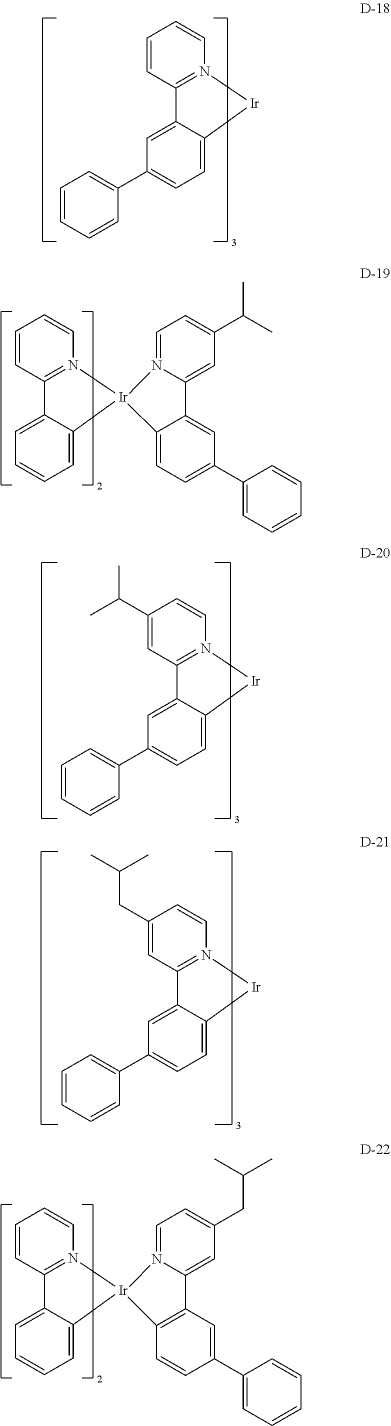

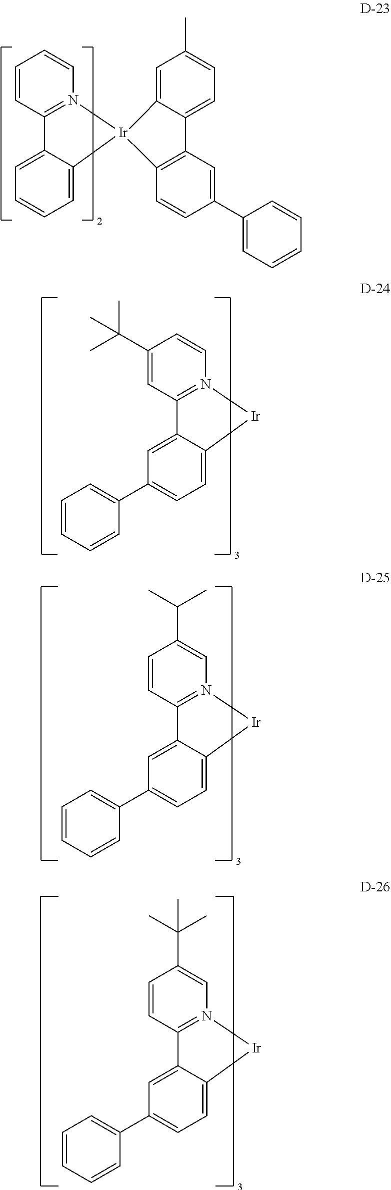

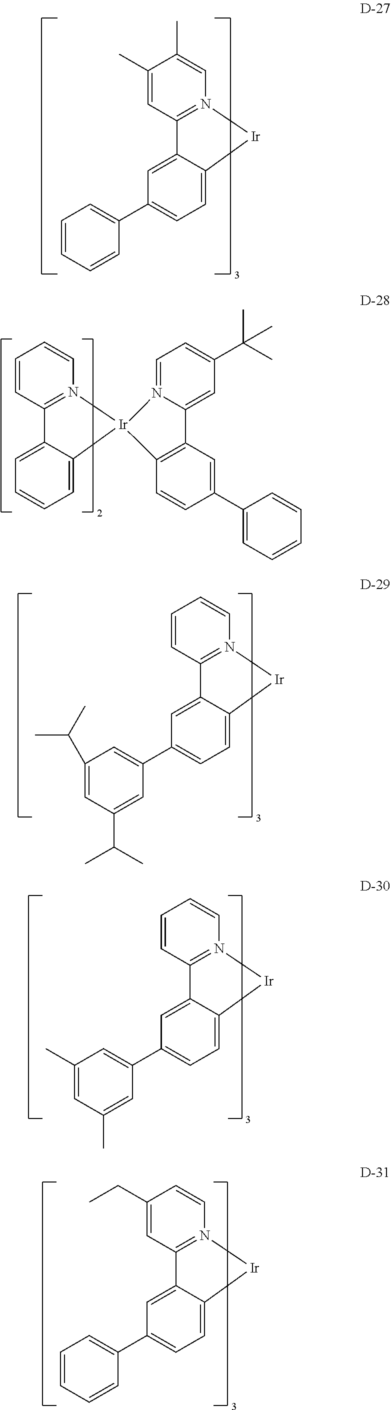

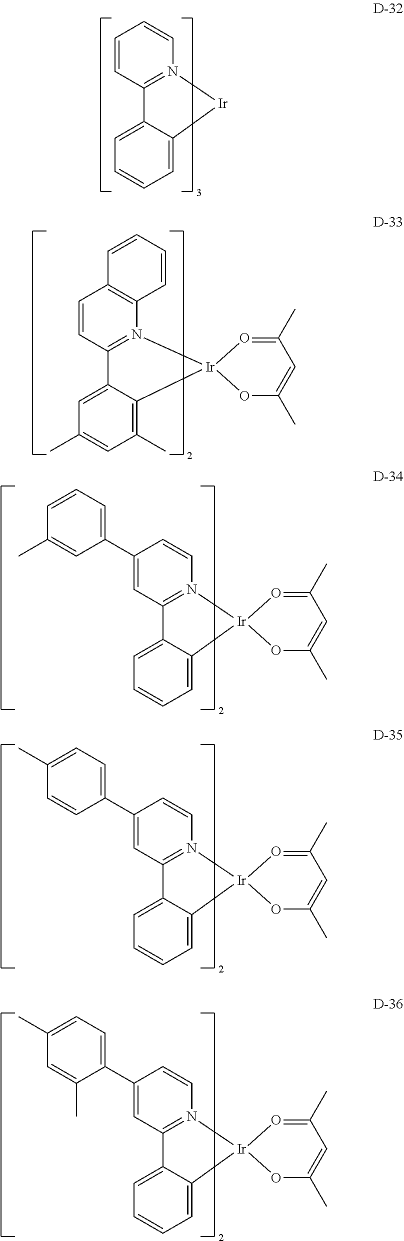

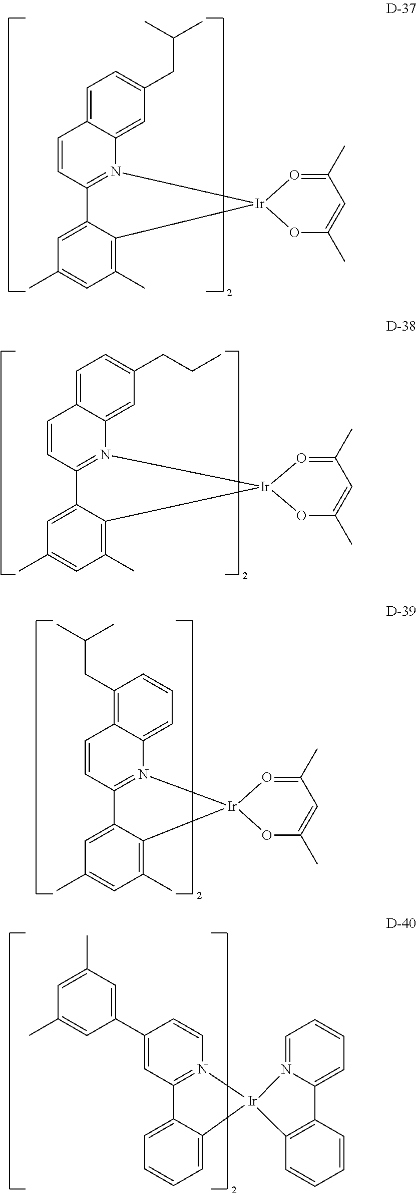

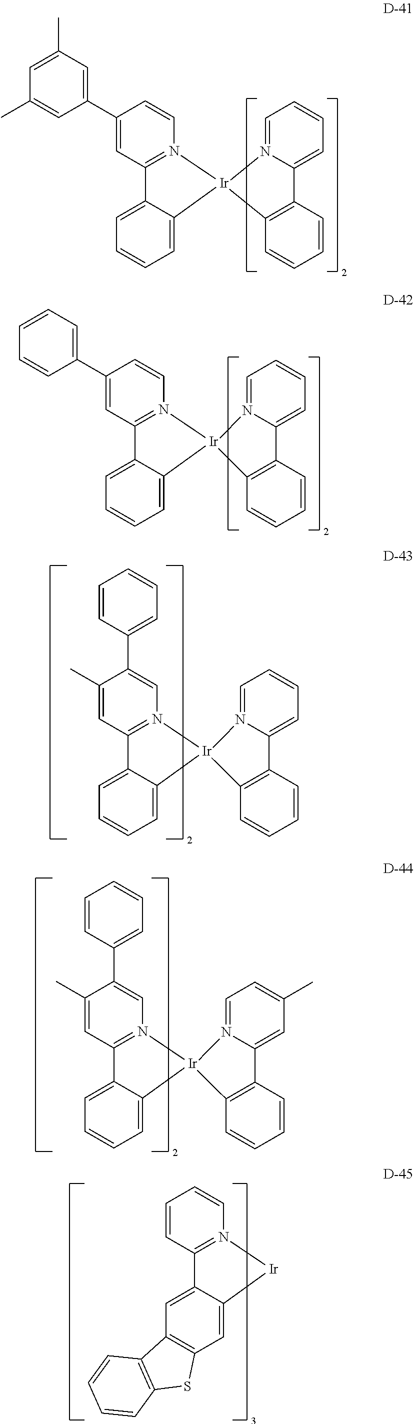

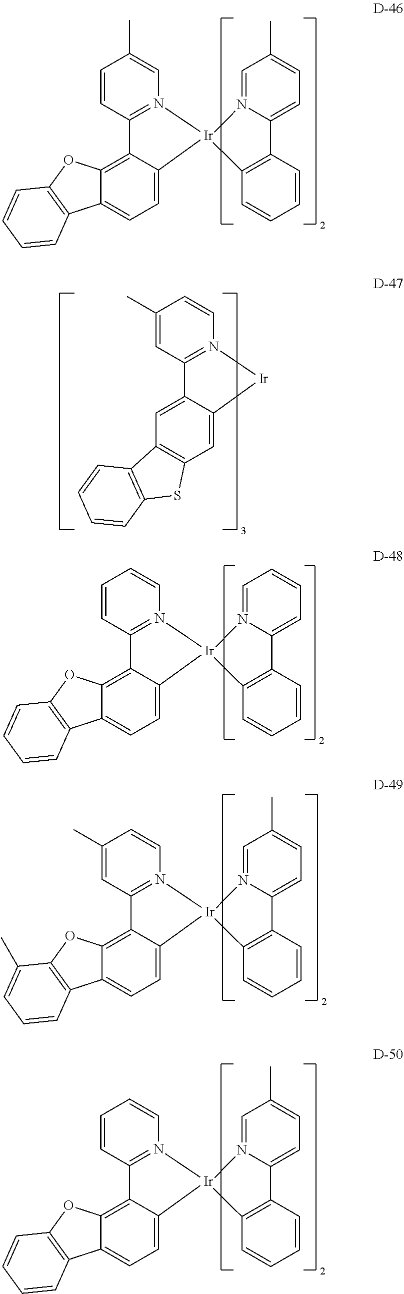

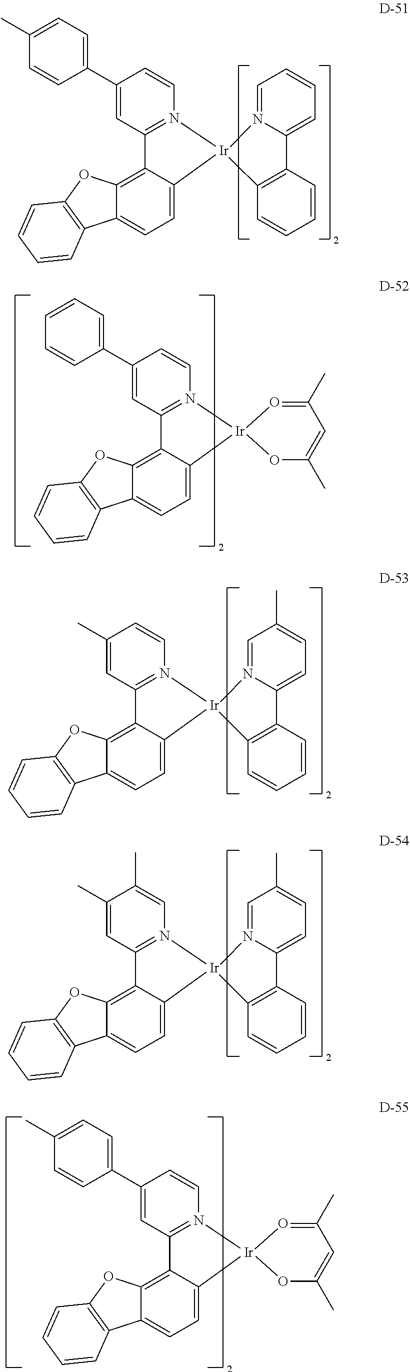

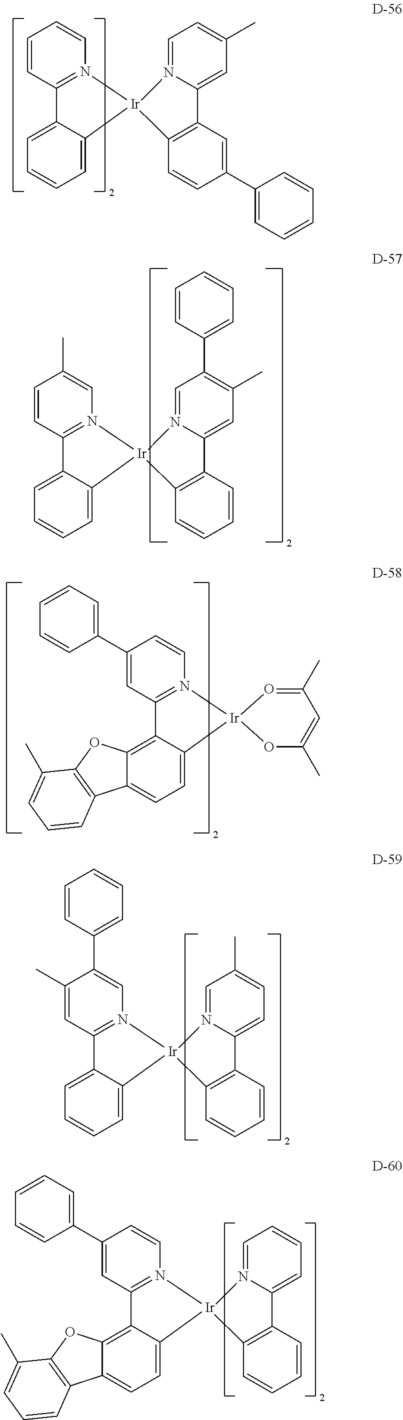

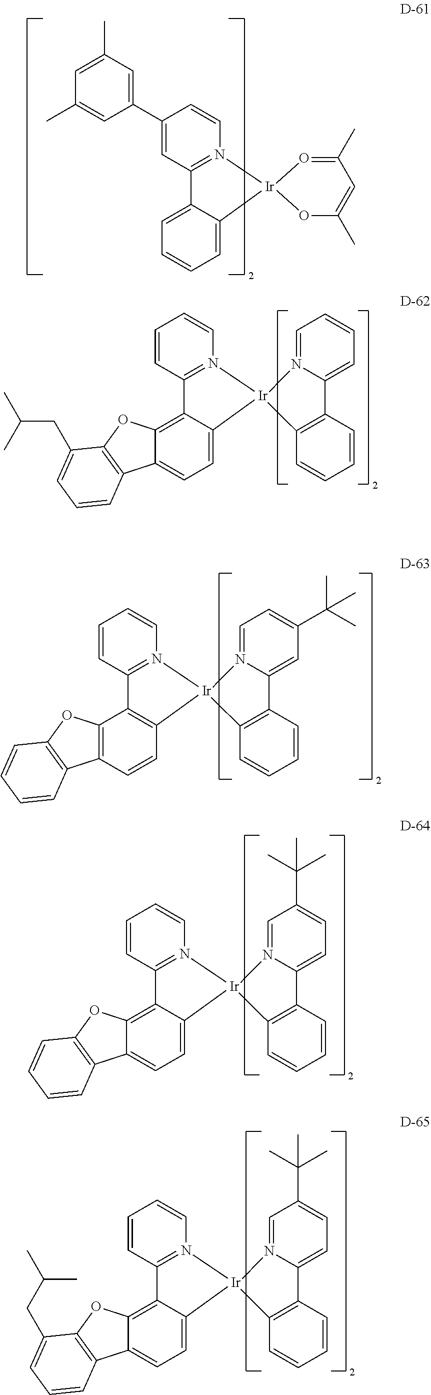

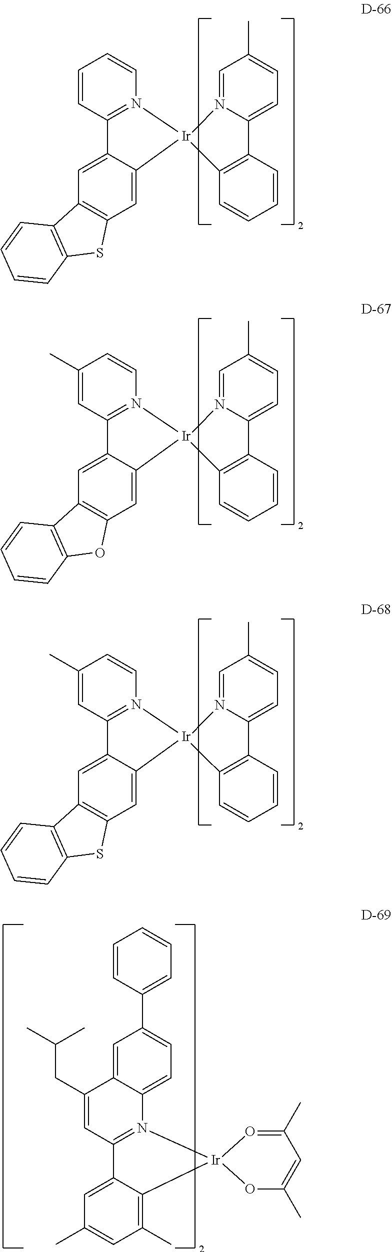

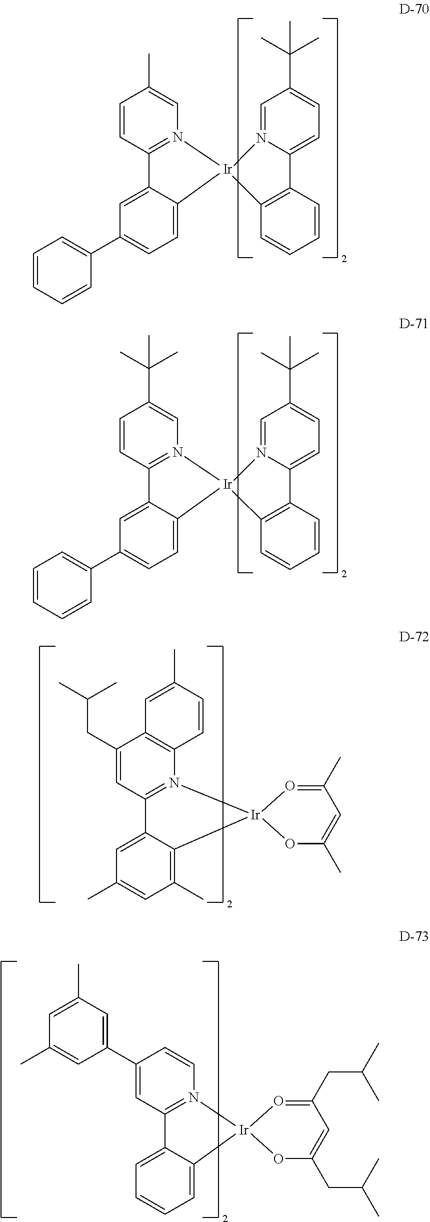

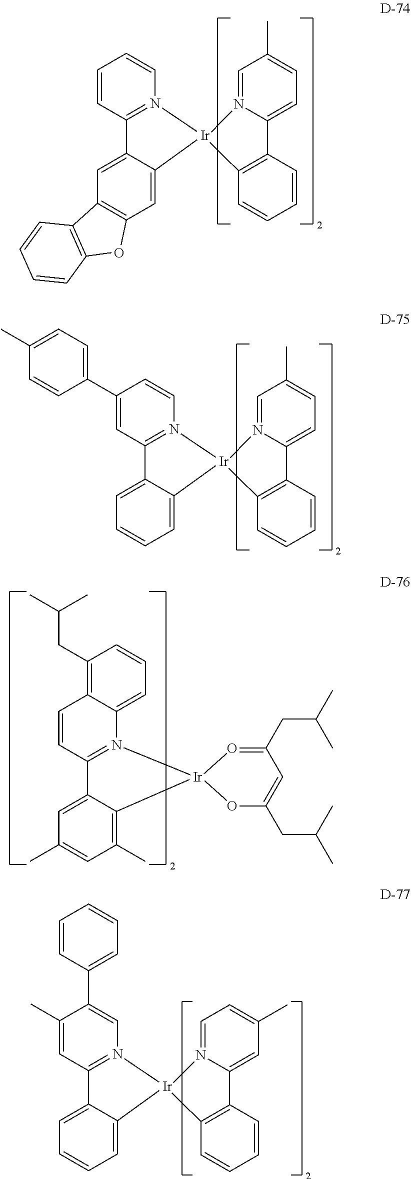

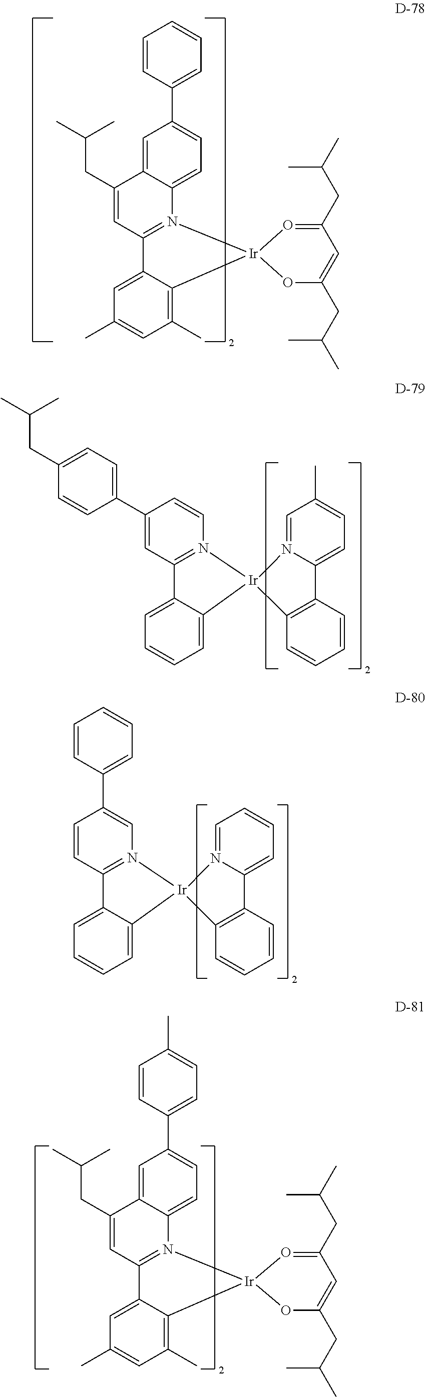

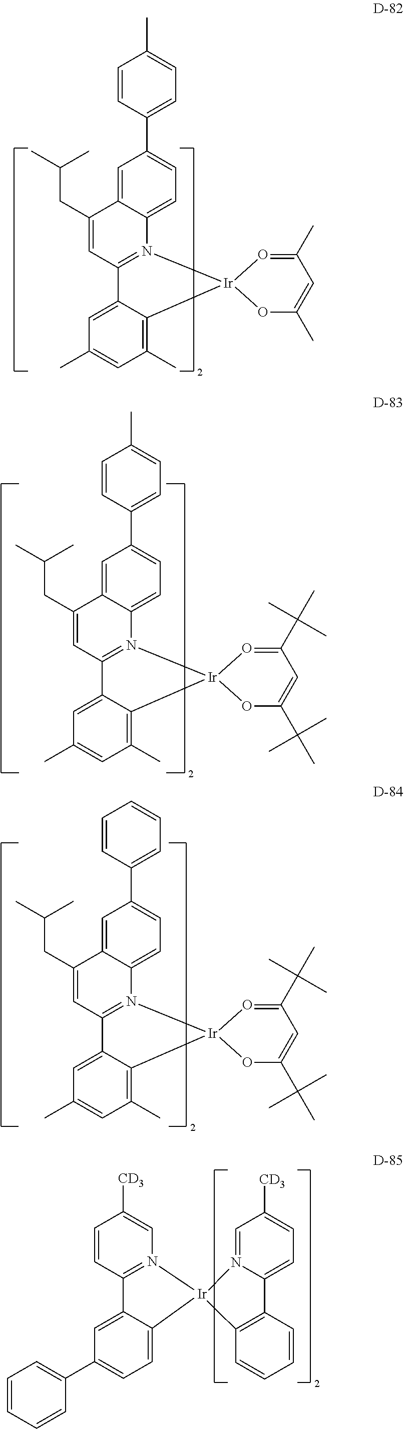

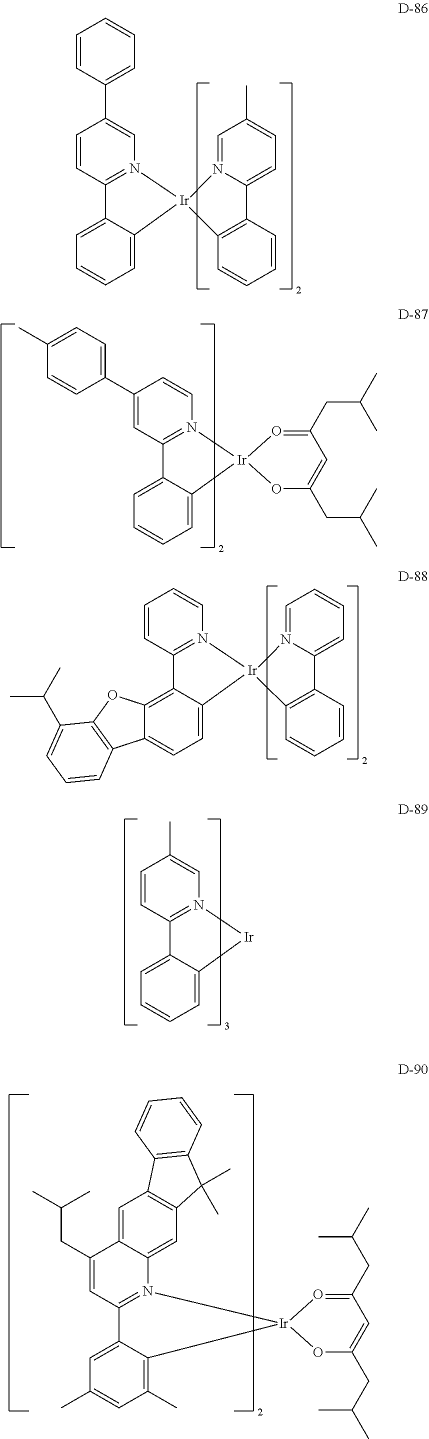

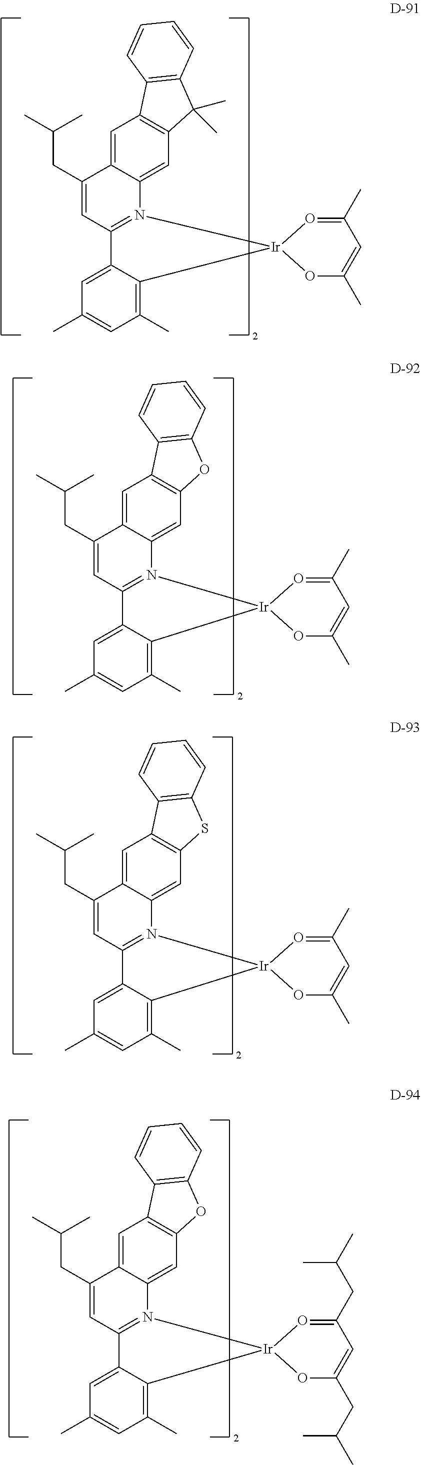

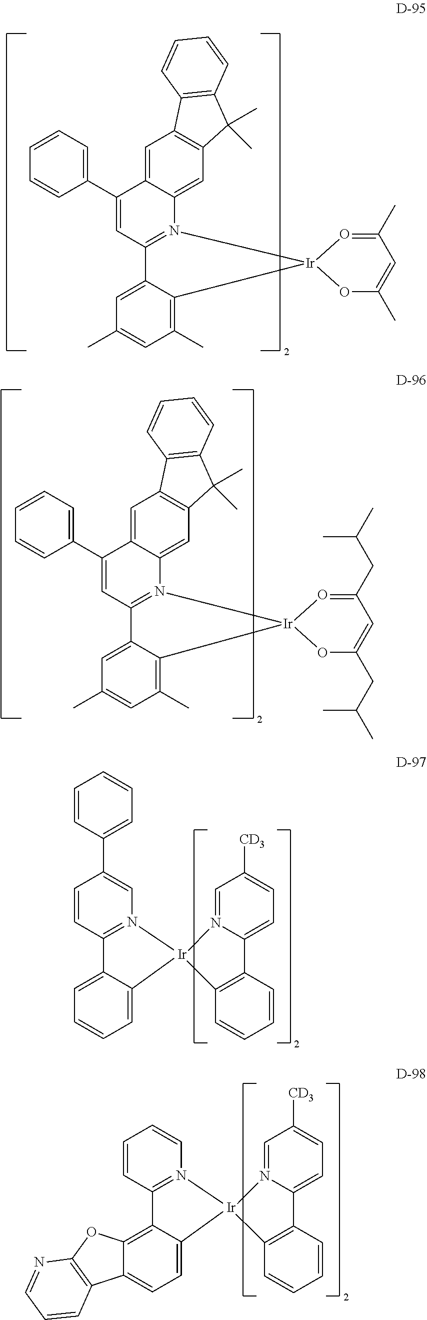

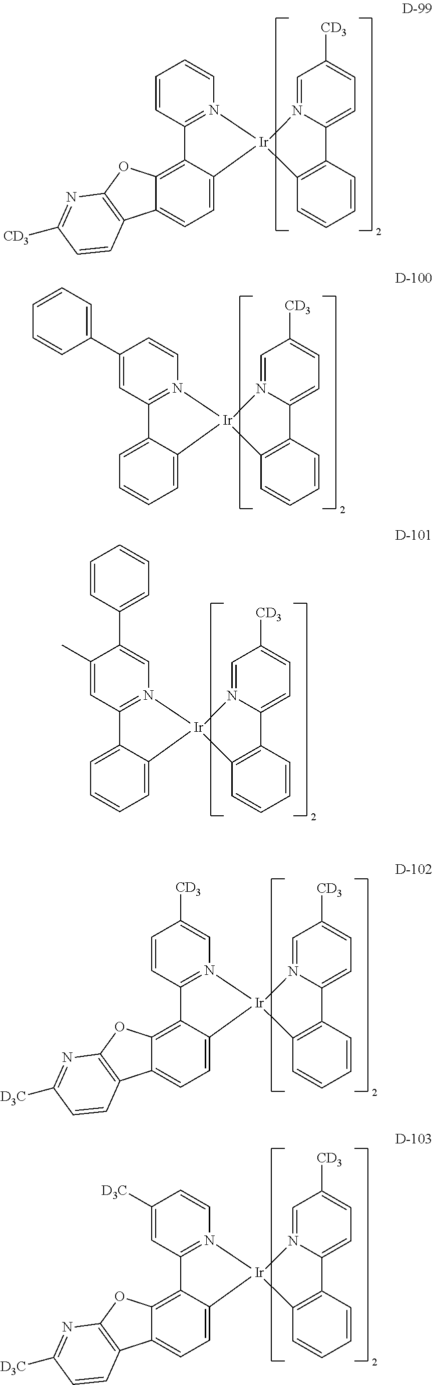

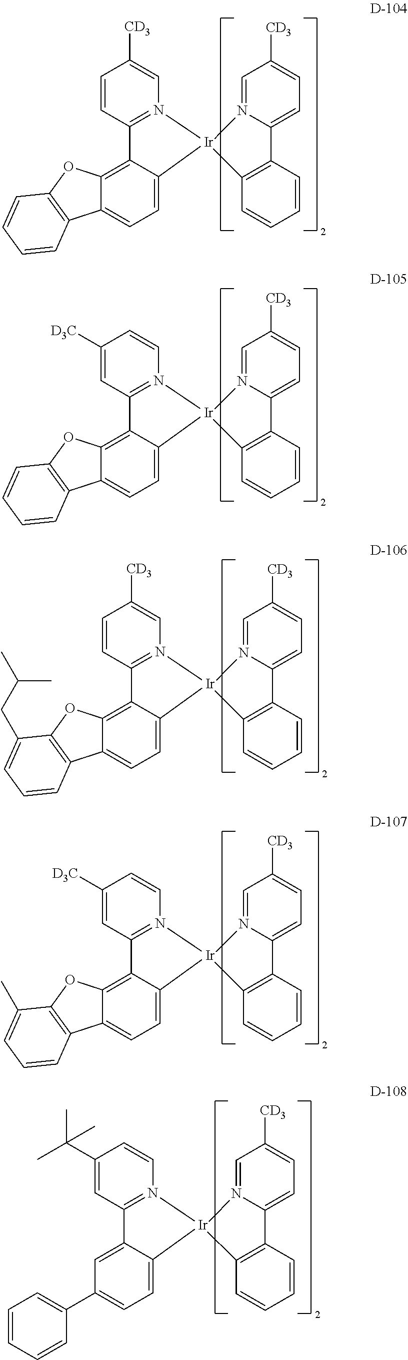

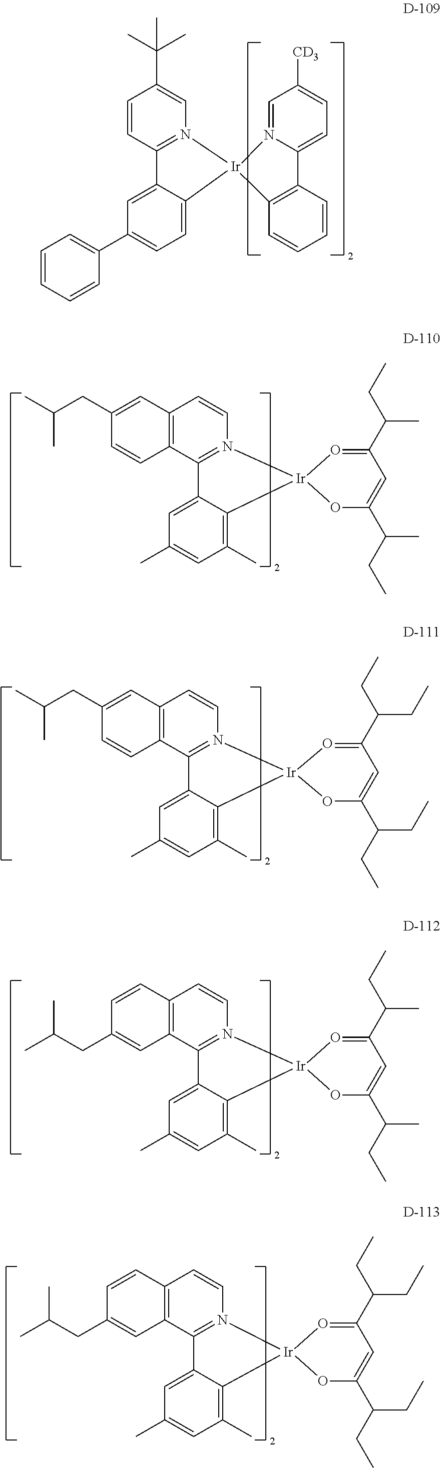

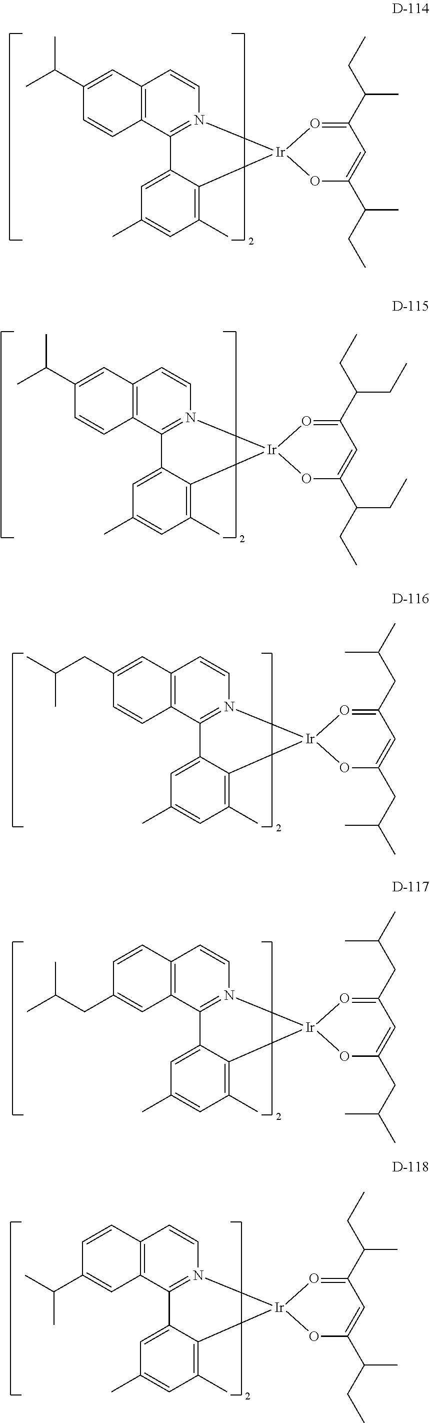

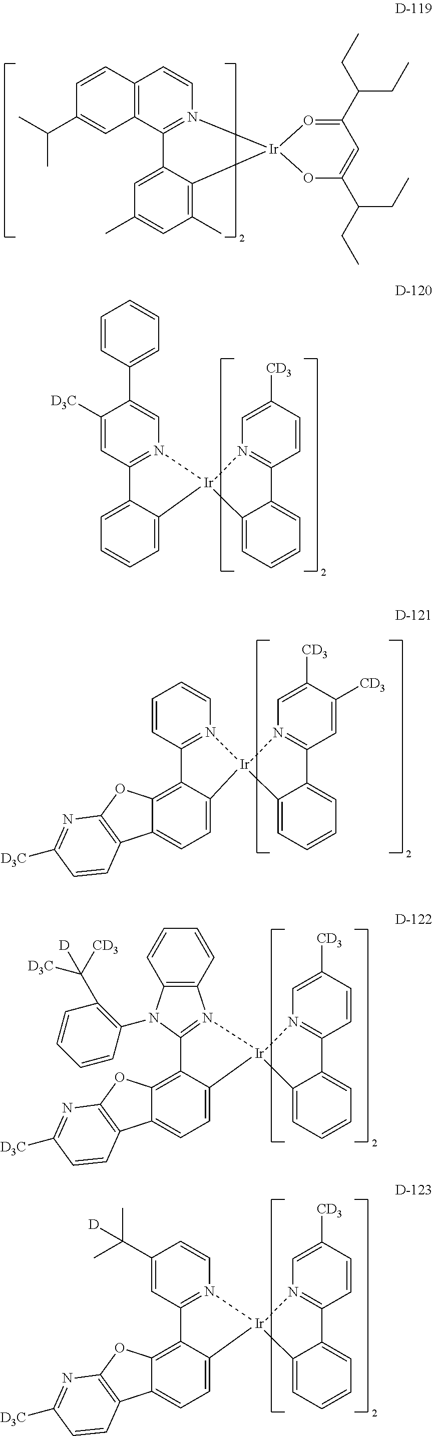

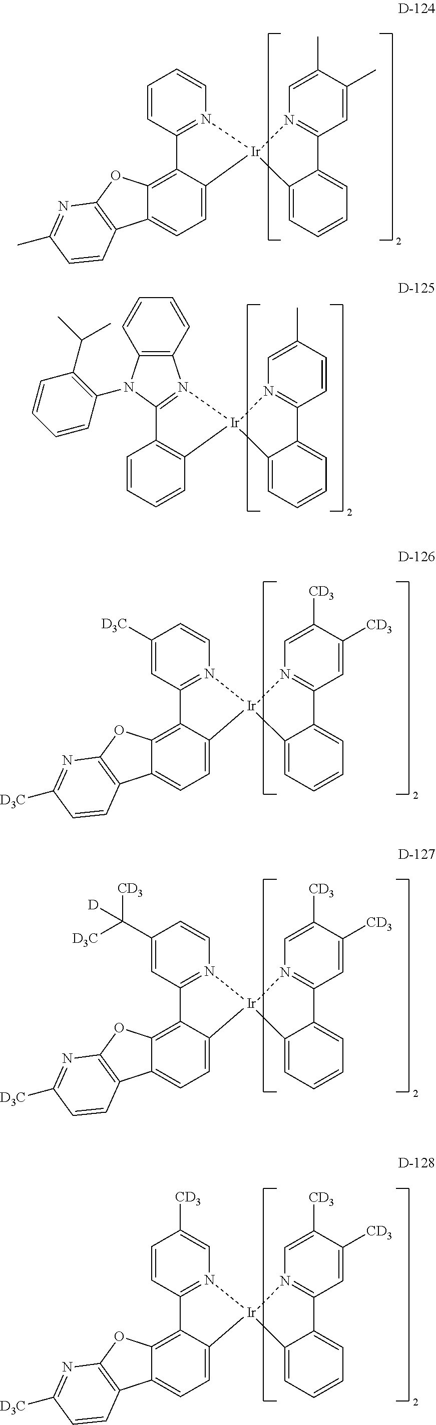

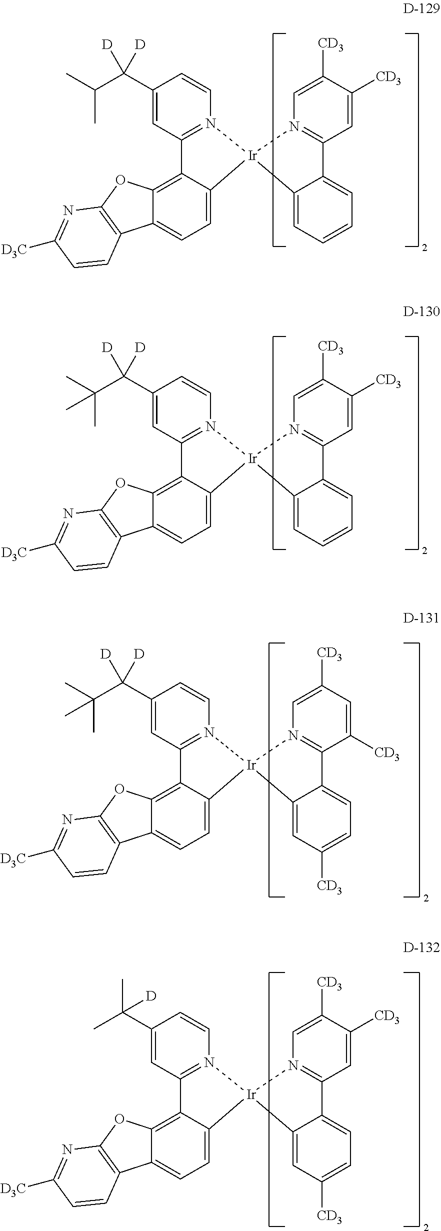

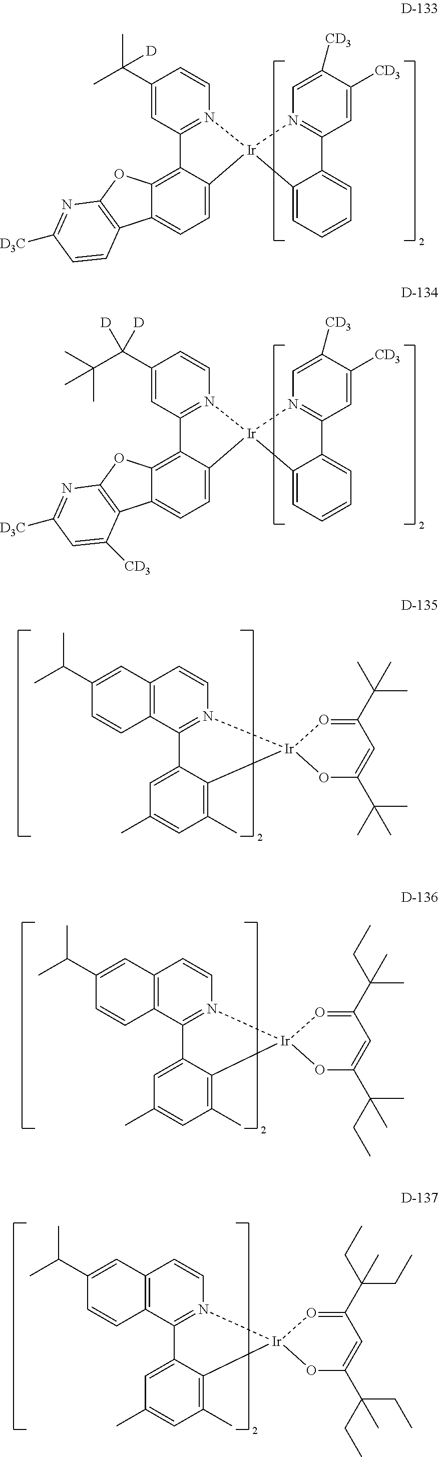

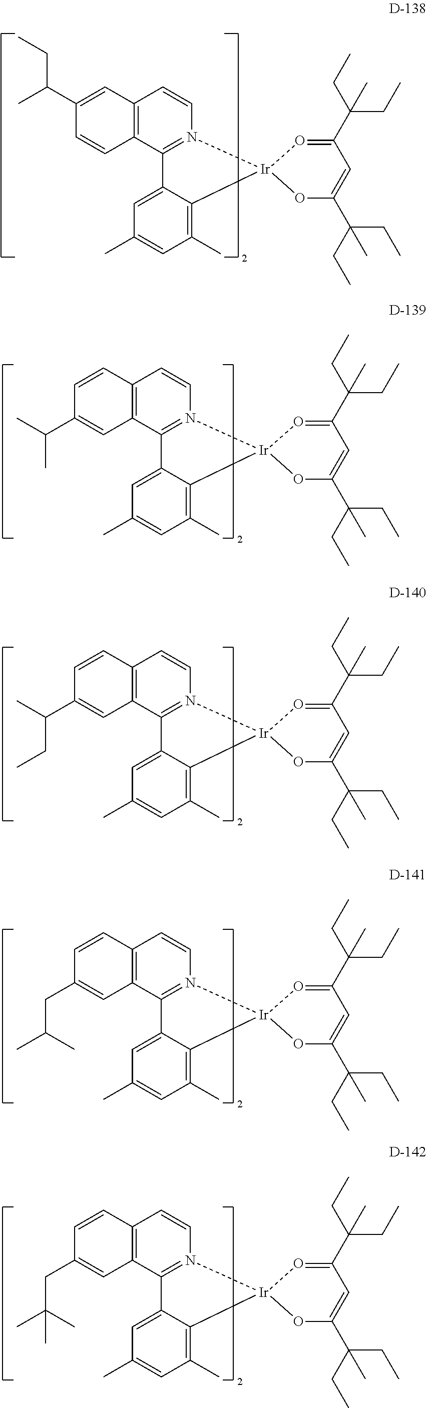

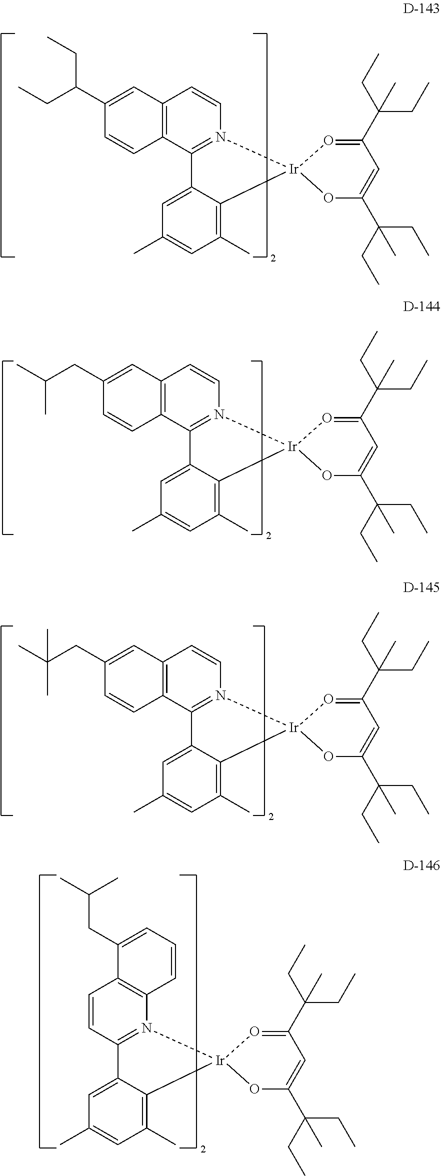

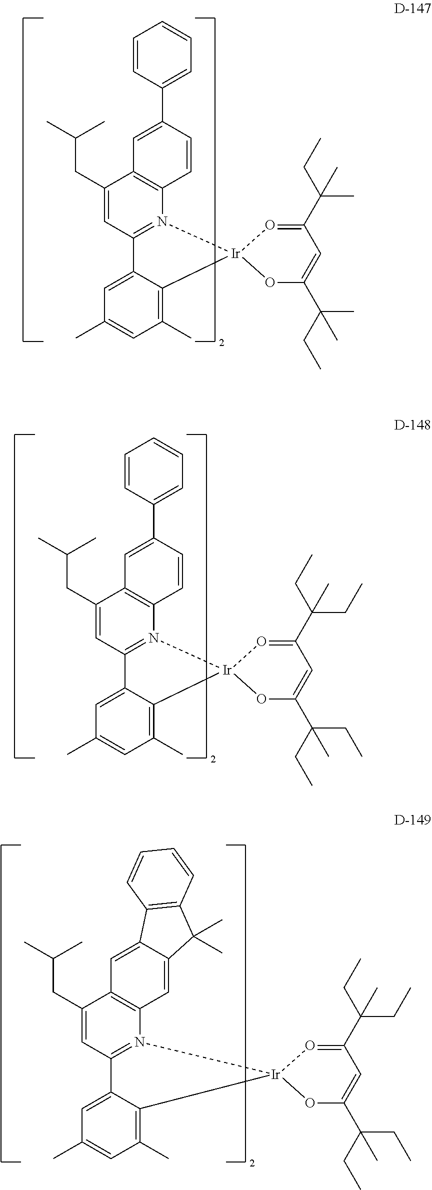

An organic electroluminescent device according to the present disclosure has an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

Further, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ (1≤X≤2), AlOₓ (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the first and second host compounds of the present disclosure are used to form a film, a co-evaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the properties of an organic electroluminescent device (OLED) comprising a plurality of host materials according to the present disclosure will be explained in detail with reference to the representative compounds of the present disclosure. However, the present disclosure is not limited by the following examples.

### Example 1: Preparation of Compound H1-1

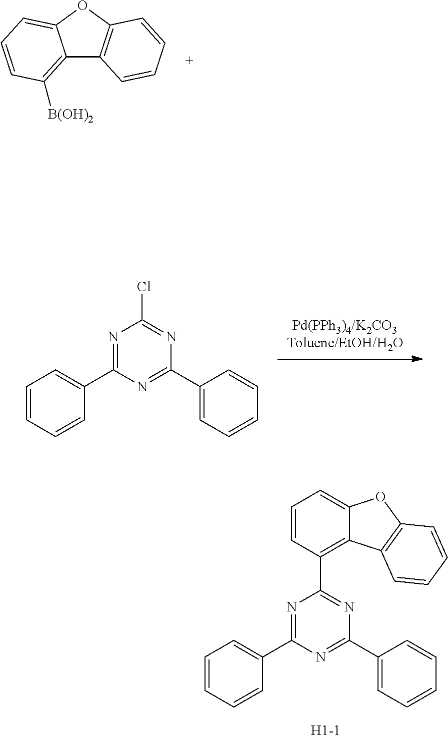 [SMILES: OB(O)c1cccc2oc3ccccc3c12 ; Clc1nc(-c2ccccc2)nc(-c2ccccc2)n1 ; c1ccc(-c2nc(-c3ccccc3)nc(-c3cccc4oc5ccccc5c34)n2)cc1]

10.5 g of 2-chloro-4,6-diphenyl-1,3,5-triazine (39.3 mmol), 10 g of dibenzo[b,d]furan-1-yl boronic acid (47.1 mmol), 2.27 g of Pd(PPh₃)₄ (1.96 mmol), 10.8 g of K₂CO₃ (78.6 mmol), 200 mL of toluene, 20 mL of EtOH, and 40 mL of H₂O were added to a flask, and stirred at 160° C. After completion of the reaction, MeOH and water were added to the mixture. The mixture was stirred, and the solvent was removed by filtration under reduced pressure. The residue was separated by column chromatography. Then, MeOH was added, and the resulting solid was filtered under reduced pressure to obtain 12.0 g of compound H1-1 (yield: 76.9%).

| | MW | M.P. |
|---|---|---|
| H1-1 | 399.45 | 220° C. |

### Example 2: Preparation of Compound H1-2

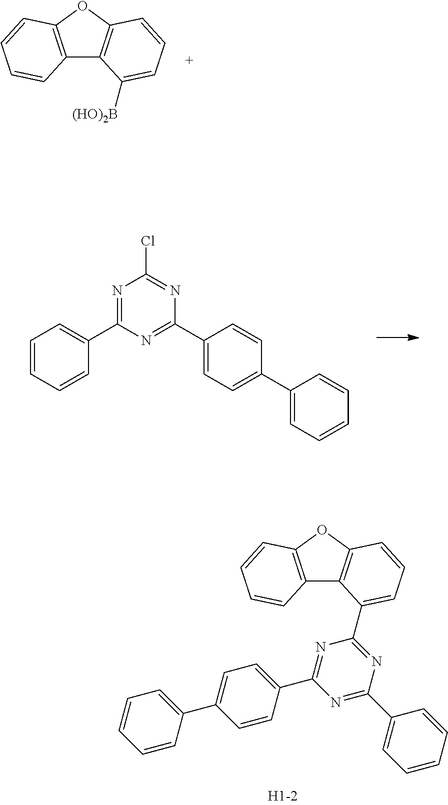 [SMILES: OB(O)c1cccc2oc3ccccc3c12 ; Clc1nc(-c2ccccc2)nc(-c2ccc(-c3ccccc3)cc2)n1 ; c1ccc(-c2ccc(-c3nc(-c4ccccc4)nc(-c4cccc5oc6ccccc6c45)n3)cc2)cc1]

In a flask, dibenzo[b,d]furan-1-yl boronic acid (2.8 g, 13.45 mmol), 2-([1,1′-biphenyl]-4-yl)-4-chloro-6-phenyl-1,3,5-triazine (4.6 g, 13.45 mmol), tetrakis(trphenylphosphine)palladium(0) (Pd(PPha)₄)(0.77 g, 0.67 mmol), and 2M potassium carbonate (4.6 g, 33.6 mmol) were dissolved in 70 mL of toluene and 17 mL of ethanol, and refluxed at 120° C. for 5 hours. After completion of the reaction, an organic layer was extracted with ethyl acetate, and the residual moisture was removed with magnesium sulfate. Then, the organic layer was dried and separated by column chromatography to obtain compound H1-2 (4.8 g, yield: 75%).

| | MW | M.P. |
|---|---|---|
| H1-2 | 475.54 | 205° C. |

### Example 3: Preparation of compound H1-142

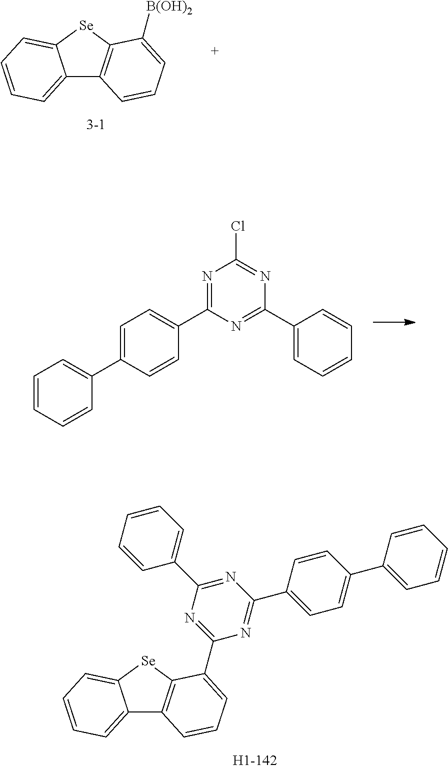 [SMILES: OB(O)c1cccc2c1[se]c1ccccc12 ; Clc1nc(-c2ccccc2)nc(-c2ccc(-c3ccccc3)cc2)n1 ; c1ccc(-c2ccc(-c3nc(-c4ccccc4)nc(-c4cccc5c4[se]c4ccccc45)n3)cc2)cc1]

Compound 3-1 (5 g, 18.1 mmol), 2-[1,1′-biphenyl]-4-yl-4-chloro-6-phenyl-1,3,5-triazine (6.2 g, 18.1 mmol), tetrakis(trphenylphosphine)palladium(0) (1.05 g, 0.909 mmol), potassium carbonate (6.2 g, 45.4 mmol), 90 mL of toluene, 23 mL of ethanol, and 23 mL of distilled water were added to a reaction vessel, and stirred under reflux for 5 hours. After completion of the reaction, the mixture was washed with distilled water, and an organic layer was extracted with ethyl acetate. The residual moisture was removed with magnesium sulfate. Then, the residue was dried and separated by column chromatography to obtain compound H1-142 (8 g. yield: 83%).

| | MW | M.P. |
|---|---|---|
| H1-142 | 538.5 | 290° C. |

### Example 4: Preparation of Compound H1-242

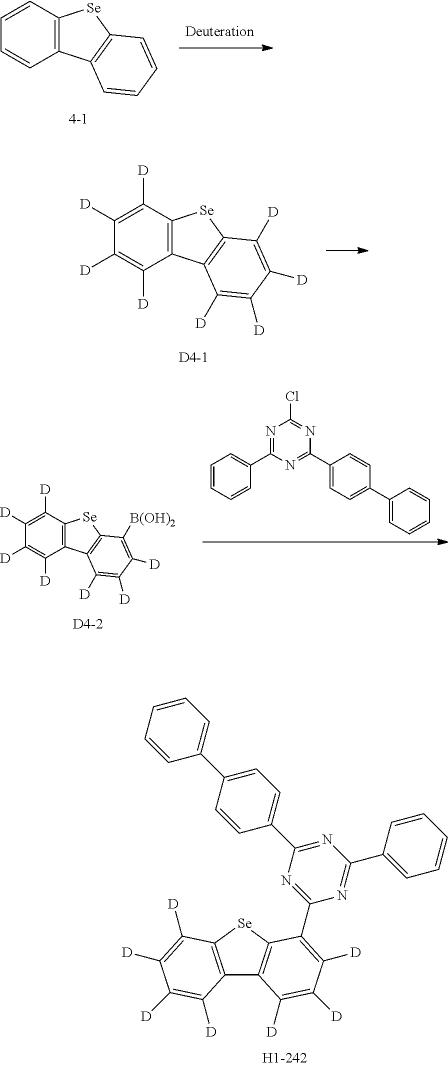

Synthesis of Compound D4-1

Compound 4-1 (8.5 g, 36.77 mmol), 85 mL of benzene-D6, and trifluoromethanesulfonic acid (8.5 mL, 96.28 mmol) were added to a reaction vessel, and stirred at 45° C. for 3 hours. The mixture was cooled to room temperature, and 8.5 mL of D₂O was added. The mixture was stirred for 10 minutes and neutralized with an aqueous K₃PO₄ solution. An organic layer was extracted with ethyl acetate, and the residual moisture was removed with magnesium sulfate. The residue was distilled under reduced pressure and separated by column chromatography to obtain compound D4-1 (8.2 g, yield: 93%).

Synthesis of Compound D4-2

Compound D4-1 (7.2 g, 30.1 mmol) and 150 mL of tetrahydrofuran were added to a reaction vessel under nitrogen atmosphere, and cooled to −78° C. Then, n-butyllithium (14 mL, 36.1 mmol) was slowly added dropwise. Trimethyl borate (8.1 mL, 72.2 mmol) was added dropwise. The mixture was allowed to react at room temperature for 12 hours. After completion of the reaction, the reaction was terminated with water, an organic layer was extracted with ethyl acetate, and the residual moisture was removed with magnesium sulfate. The residue was dried and separated by column chromatography to obtain compound D4-2 (1.7 g, yield: 20%).

Synthesis of Compound H1-242

Compound D4-2 (1.7 g, 6.03 mmol), 2-[1,1′-biphenyl]-4-yl-4-chloro-6-phenyl-1,3,5-triazine (2.1 g, 6.33 mmol), tetrakis(trphenylphosphine)palladium(0) (0.35 g, 0.30 mmol), potassium carbonate (2.1 g, 15.1 mmol), 30 mL of toluene, 8 mL of ethanol, and 8 mL of distilled water were added to a reaction vessel, and stirred under reflux for 4 hours. After completion of the reaction, the mixture was washed with distilled water, and an organic layer was extracted with ethyl acetate. The residual moisture in the organic layer was removed with magnesium sulfate. The residue was dried and separated by column chromatography to obtain compound H1-242 (2.1 g, yield: 65%).

| | MW | M.P. |
|---|---|---|
| H1-242 | 545.5 | 290° C. |

### Example 5: Preparation of Compound H1-271

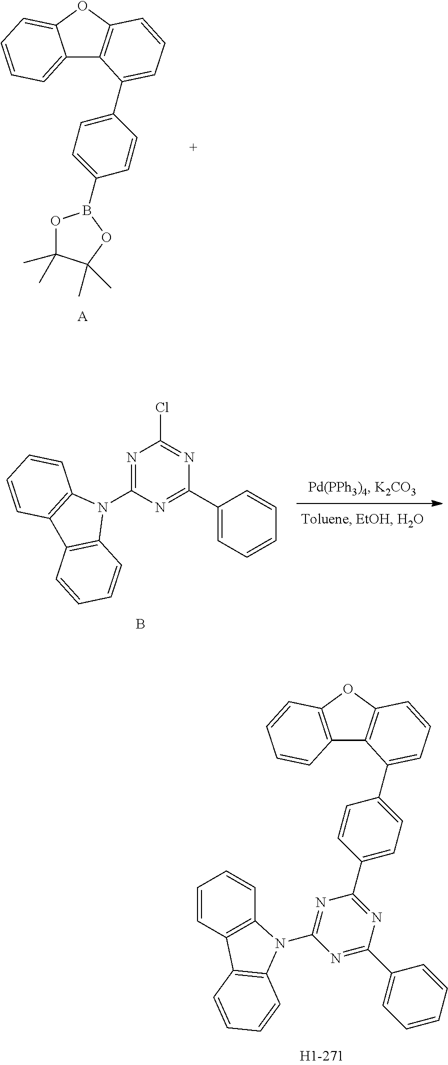

In a flask, compound A (6.0 g, 16.20 mmol), compound B (6.4 g, 17.83 mmol), Pd(PPh₃)₄ (0.9 g, 0.81 mmol), and K₂CO₃ (4.5 g, 32.4 mmol) were dissolved in 81 mL of toluene, 20 mL of EtOH, and 20 mL of H₂O, and stirred under reflux for 4 hours. The mixture was cooled to room temperature. H₂O was added to the reactant containing the produced solid. The mixture was stirred for 30 minutes, filtered, and separated by column chromatography to obtain compound H1-271 (7.0 g, yield: 77%).

| | MW | M.P. |
|---|---|---|
| H1-271 | 564.65 | 253° C. |

### Example 6: Preparation of Compound H1-192

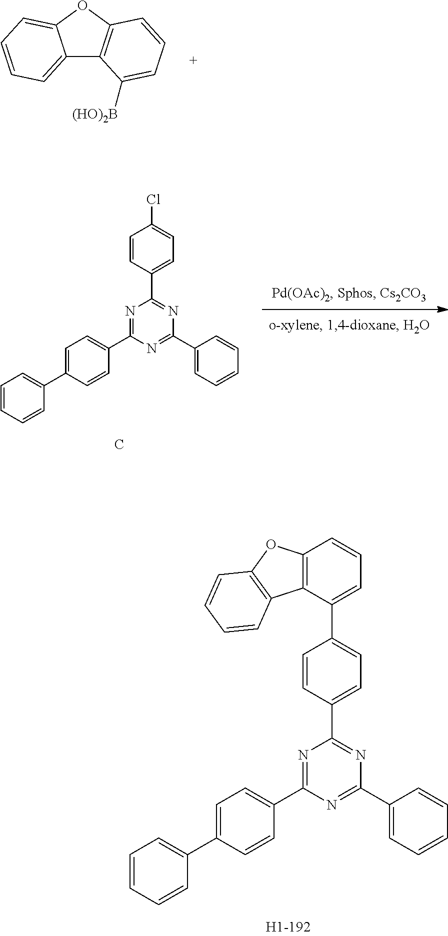

In a flask, dibenzo[b,d]furan-1-yl boronic acid (5 g, 18.36 mmol), compound C (10.0 g, 23.81 mmol), Pd(OAc)₂ (535 mg, 2.38 mmol), Sphos (1.9 g, 4.76 mmol), and Cs₂CO₃ (23 g, 71.4 mmol) were dissolved in 120 mL of o-xylene, 30 mL of 1,4-dioxane, and 30 mL of H₂O, and stirred under reflux for 3 hours. The mixture was cooled to room temperature. H₂O was added to the reactant containing the produced solid. The mixture was stirred for 30 minutes, filtered, and separated by column chromatography to obtain compound H1-192 (7.1 g, yield: 54%).

| | MW | M.P. |
|---|---|---|
| H1-192 | 551.65 | 188° C. |

### Example 7: Preparation of Compound H1-281

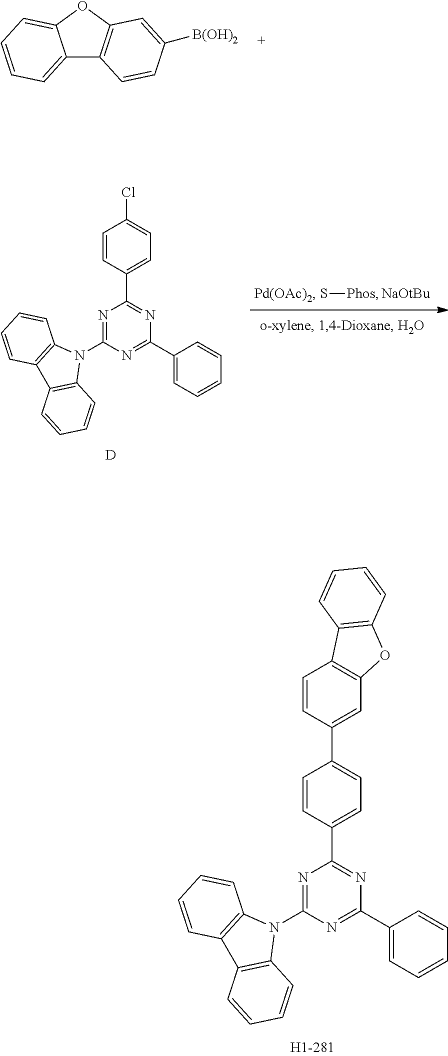

In a flask, dibenzo[b,d]furan-3-yl boronic acid (11.1 g, 25.72 mmol), compound D (6.0 g, 28.3 mmol), Pd(OAc)₂ (0.57 g, 2.57 mmol), S-Phos (0.52 g, 5.45 mmol), and NaOtBu (7.4 g, 77.16 mmol) were dissolved in 130 mL of o-xylene, 32 mL of 1,4-Dioxane, and 32 mL of H₂O, and stirred under reflux for 4 hours. The mixture was cooled to room temperature. H₂O was added to the reactant containing the produced solid. The mixture was stirred for 30 minutes, filtered, and separated by column chromatography to obtain compound H1-281 (7.8 g, yield: 53%).

| | MW | M.P. |
|---|---|---|
| H1-281 | 564.65 | 308° C. |

### Example 8: Preparation of Compound H1-275

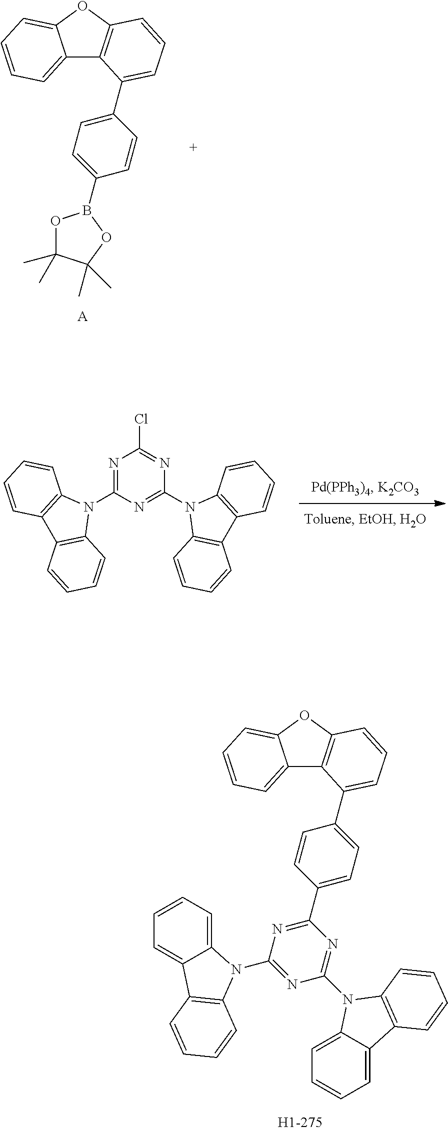

In a flask, compound A (3.8 g, 9.45 mmol), 9,9′-(6-chloro-1,3,5-triazine-2,4-diyl)bis(9H-carbazole) (3.8 g, 8.59 mmol), Pd(PPh₃)₄ (0.5 g, 0.42 mmol), and K₂CO₃ (2.9 g, 21.47 mmol) were dissolved in 40 mL of toluene, 20 mL of EtOH, and 20 mL of H₂O, and stirred under reflux for 4 hours. The mixture was cooled to room temperature. H₂O was added to the reactant containing the produced solid. The mixture was stirred for 30 minutes, filtered, and separated by column chromatography to obtain compound H1-275 (4.2 g, yield: 75%).

| | MW | M.P. |
|---|---|---|
| H1275 | 653.75 | 311° C. |

### Example 9: Preparation of Compound H1-277

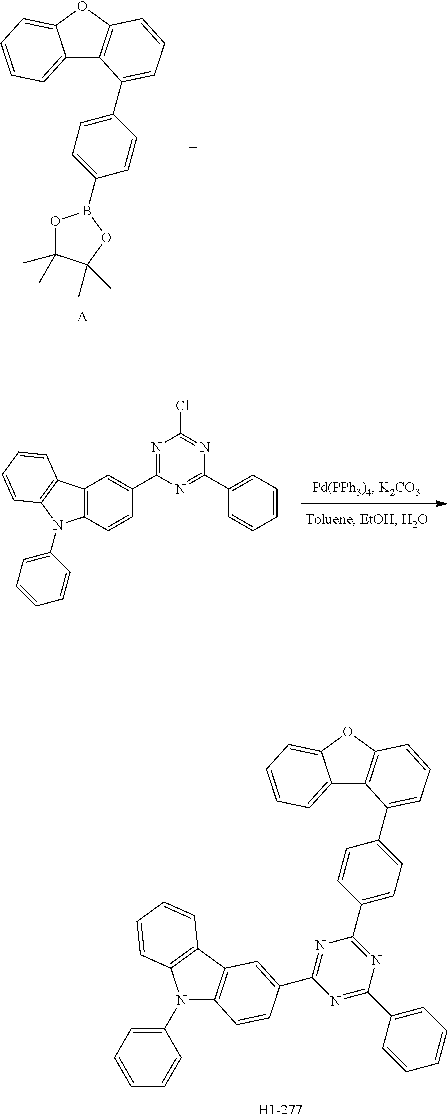

In a flask, compound A (3.7 g, 8.59 mmol), 3-(4-chloro-6-phenyl-1,3,5-triazin-2-yl)-9-phenyl-9H-carbazole (3.5 g, 9.45 mmol), Pd(PPh₃)₄ (0.5 g, 0.42 mmol), and K₂CO₃ (2.9 g, 11.06 mmol) were dissolved in 40 mL of toluene, 20 mL of EtOH, and 20 mL of H₂O, and stirred under reflux for 4 hours. The mixture was cooled to room temperature. H₂O was added to the reactant containing the produced solid. The mixture was stirred for 30 minutes, filtered, and separated by column chromatography to obtain compound H1-277 (2.8 g, yield: 51%).

| | MW | M.P. |
|---|---|---|
| H1-277 | 640.75 | 280° C. |

### Example 10: Preparation of Compound H1-262

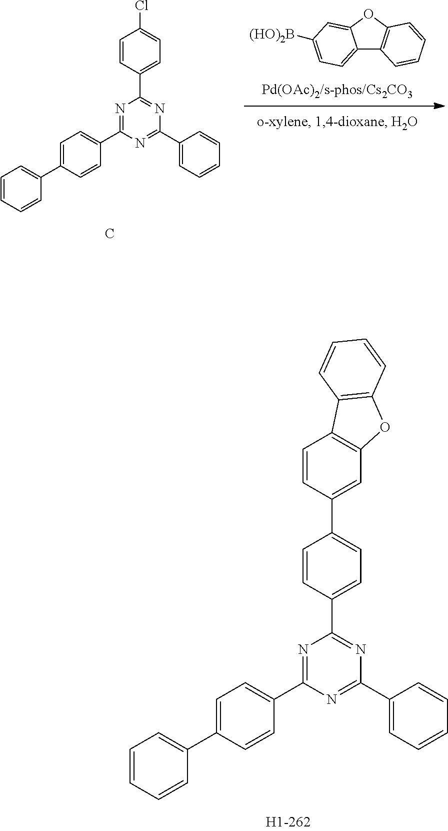

In a flask, compound C (13.2 g, 31.44 mmol), dibenzo[b,d]furan-3-yl boronic acid (8.0 g, 37.73 mmol), Pd(OAc)₂ (0.7 g, 3.14 mmol), s-Phos (2.6 g, 6.28 mmol), and Cs₂CO₃ (30.7 g, 94.32 mmol) were dissolved in 157 mL of o-xylene, 40 mL of 1,4-dioxane, and 40 mL of H₂O, and stirred under reflux for 4 hours. The mixture was cooled to room temperature. H₂O was added to the reactant containing the produced solid. The mixture was stirred for 30 minutes, filtered, and separated by column chromatography to obtain compound H1-262 (6.7 g, yield: 39%).

| | MW | M.P. |
|---|---|---|
| H1-262 | 551.64 | 255° C. |

### Example 11: Preparation of Compound H2-50-D25

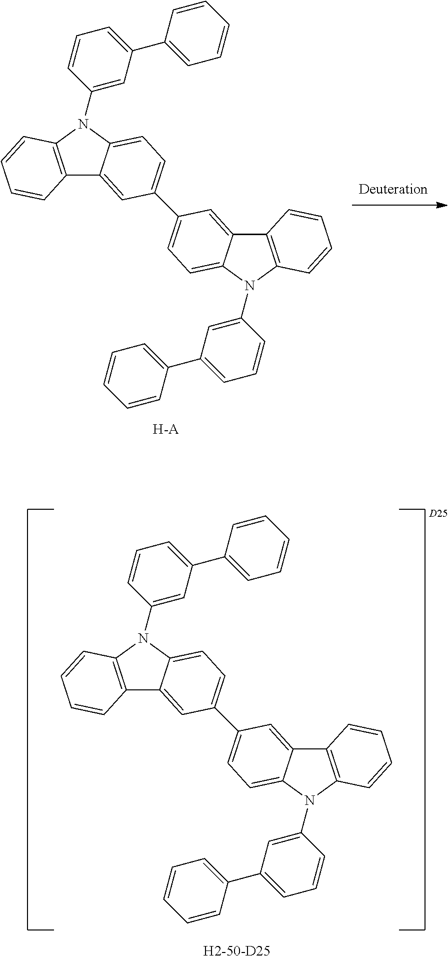

In a flask, compound H-A (15.0 g, 42.9 mmol) was dissolved in 900 mL of benzene-D6 by heat. Triflic acid (25.4 mL, 169.5 mmol) was added at 60° C. After 3 hours, the mixture was cooled to room temperature, and 30 mL of D₂O was added. The mixture was stirred for 10 minutes, and neutralized with an aqueous K₃PO₄ solution. An organic layer was extracted with ethyl acetate, and the residual moisture was removed with magnesium sulfate. The residue was distilled under reduced pressure and separated by column chromatography to obtain compound H2-50-025 (12 g, yield: 77.0%).

| | MW | M.P. |
|---|---|---|
| H2-50-D25 | 661 | 152° C. |

### Example 12: Preparation of Compound H2-2-D22

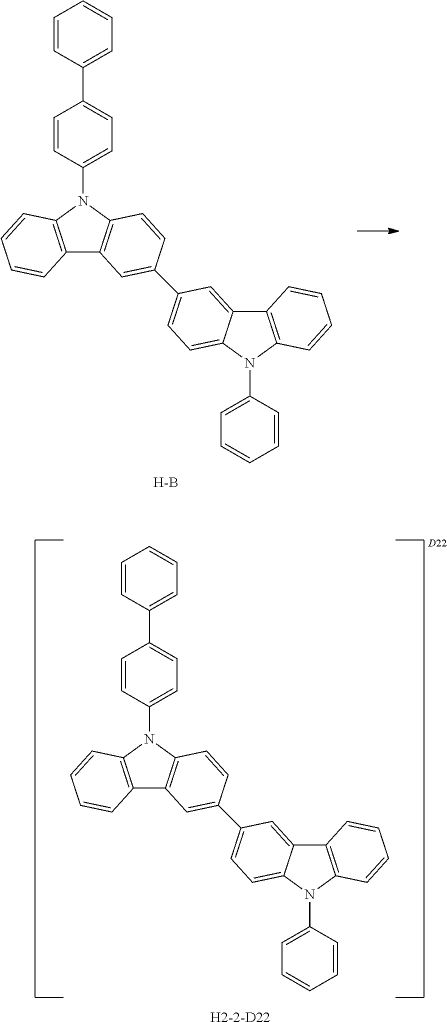

In a flask, compound H-B (25.0 g, 44.62 mmol) was dissolved in 600 mL of benzene-D6 by heat. Triflic acid (75 mL, 834.6 mmol) was added at 40° C. After 3 hours and 30 minutes, the mixture was cooled to room temperature, and 25 mL of D₂O was added. The mixture was stirred for 10 minutes, and neutralized with an aqueous K₃PO₄ solution. An organic layer was extracted with ethyl acetate, distilled under reduced pressure, separated by column chromatography, and recrystallized with toluene to obtain compound H2-2-D22 (17.8 g, yield: 68.56%).

| | MW | M.P. |
|---|---|---|
| H2-2-D22 | 582.23 | 262° C. |

### Example 13: Preparation of Compound H1-356

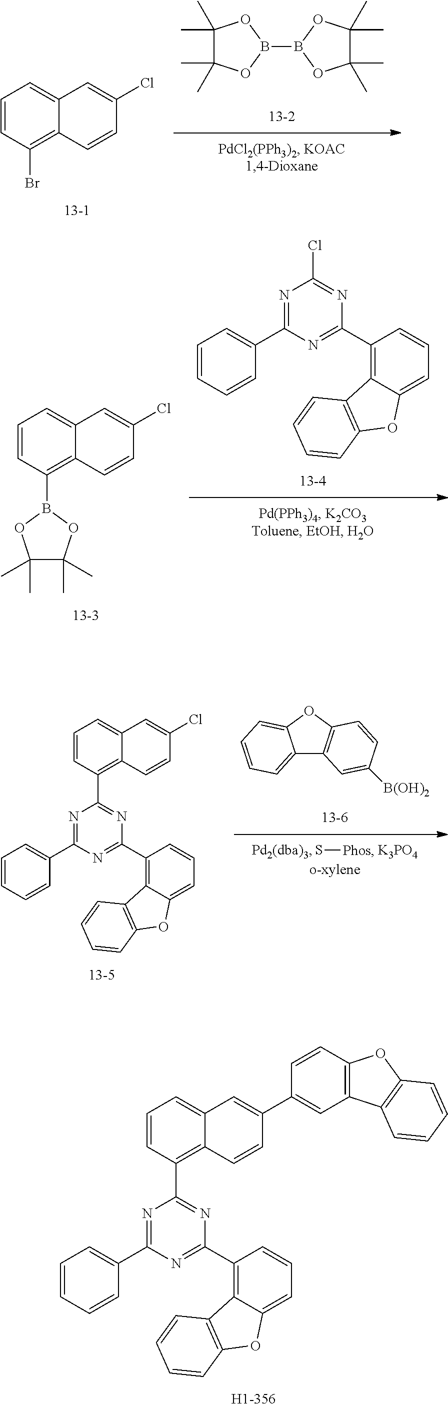

Synthesis of Compound 13-3

Compound 13-1 (20.0 g, 82.81 mmol), compound 13-2 (25.2 g, 99.37 mmol), PdCl₂(PPh₃)₂ (1.2 g, 1.66 mmol), and KOAc (16.3 g, 165.62 mmol) were dissolved in 415 mL of 1,4-dioxane, and stirred under reflux for 3 hours. The mixture was cooled to room temperature, layer-separated (MC/H₂O), celite filtered, and silica filtered to obtain compound 13-3 (24.3 g, yield: 101.7%) in an oil phase.

Synthesis of Compound 13-5

Compound 13-3 (24.3 g, 84.20 mmol), compound 13-4 (33.1 g, 92.62 mmol), Pd(PPha)₄ (4.9 g, 4.21 mmol), and K₂CO₃ (23.3 g, 168.4 mmol) were dissolved in 420 mL of toluene, 105 mL of EtOH, and 105 mL of H₂O, and stirred under reflux for 3 hours. The mixture was cooled to room temperature. H₂O was added to the reactant containing the produced solid. The mixture was stirred for 30 minutes, filtered, and separated by column chromatography to obtain compound 13-5 (12.1 g, yield: 29.7%).

Synthesis of Compound H1-356

Compound 13-5 (5.0 g, 10.33 mmol), compound 13-6 (3.9 g, 18.59 mmol), Pd₂(dba)₃ (0.5 g, 0.52 mmol), S-Phos (0.4 g, 1.03 mmol), and K₃PO₄ (5.5 g, 25.82 mmol) were dissolved in 52 mL of o-xylene, and stirred under reflux for 2 hours. The mixture was cooled to room temperature. H₂O was added to the reactant containing the produced solid, and stirred for 30 minutes. Then, the solid was filtered. The filtrate was separated by column chromatography, and recrystallized to obtain compound H1-356 (4.4 g, yield: 69.1%).

| | MW | M.P. |
|---|---|---|
| H1-356 | 615.69 | 235° C. |

### Example 14: Preparation of Compound H1-343

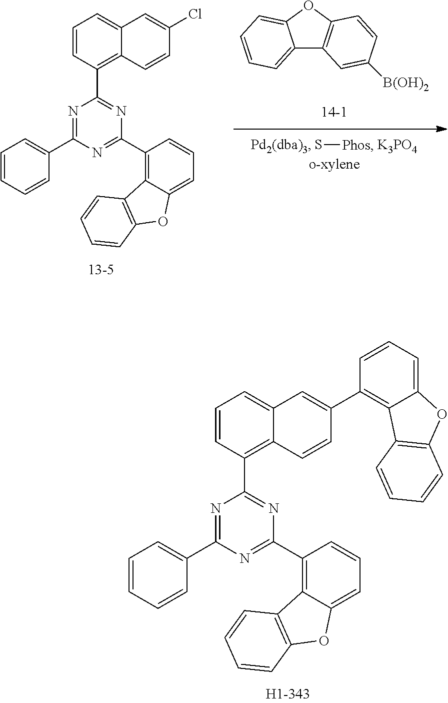

Compound 13-5 (5.0 g, 10.33 mmol), compound 14-1 (4.4 g, 20.66 mmol), Pd₂(dba)₃ (0.5 g, 0.52 mmol), S-Phos (0.4 g, 1.03 mmol), and K₃PO₄ (5.5 g, 25.82 mmol) were dissolved in 52 mL of o-xylene, and stirred under reflux for 2 hours. The mixture was cooled to room temperature. H₂O was added to the reactant containing the produced solid, and the mixture was stirred for 30 minutes. Then, the solid was filtered. The filtrate was separated by column chromatography, and recrystallized to obtain compound H1-343 (2.4 g, yield: 37.7%).

| | MW | M.P. |
|---|---|---|
| H1-343 | 615.69 | 238° C. |

### Device Examples 1 to 3: Producing an OLED Comprising a Plurality of Host Materials According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: the first and second host materials shown in Table 1 below were introduced into two cells of the vacuum vapor deposition apparatus as hosts, and compound D-130 was introduced into another cell as a dopant. The two host materials were evaporated at a different rate of 2:1 and the dopant material was simultaneously evaporated at a different rate, and the dopant was deposited in a doping amount of 10 wt % based on the total amount of the host and the dopant to form a light-emitting layer having a thickness of 40 nm on the second hole transport layer. Compound ET-1 and compound EI-1 were evaporated in a weight ratio of 40:60 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Comparative Compound as a Host

An OLED was produced in the same manner as in Device Example 1, except that compound H1-1 (the first host) and compound H-A (the second host) were used as hosts of the light-emitting layer.

The driving voltage, luminous efficiency, and light-emitting color at a luminance of 1,000 nit, and the time taken for luminance to decrease from 100% to 95% at a luminance of 20,000 nit (lifetime; T95) of the OLEDs produced in Device Examples 1 to 3 and Comparative Example 1 are provided in Table 1 below.

TABLE 1

| | | | Driving | Luminous | Light- | |
|---|---|---|---|---|---|---|
| | First | Second | Voltage | Efficiency | Emitting | Lifetime |
| | Host | Host | [V] | [cd/A] | Color | (T95)[hr] |
| Device | H1-1 | H2-50-D25 | 2.8 | 105.6 | Green | 154 |
| Example 1 | | | | | | |
| Device | H1-2 | H2-50-D25 | 2.7 | 103.5 | Green | 193 |
| Example 2 | | | | | | |
| Device | H1-142 | H2-50-D25 | 2.7 | 104.3 | Green | 158 |
| Example 3 | | | | | | |
| Comparative | H1-1 | H-A | 2.9 | 105.8 | Green | 129 |
| Example 1 | | | | | | |

### Device Examples 4 to 17: Producing an OLED Comprising a Plurality of Host Materials According to the Present Disclosure

An OLED was produced in the same manner as in Device Example 1, except that compound HT-3 were used instead of compound HT-2 as the second hole transport layer, and the first host compounds and the second host compounds shown in Tables 2 and 3 below as hosts of the light-emitting layer.

### Comparative Examples 2 to 15: Producing an OLED Comprising a Comparative Compound as a Host

OLEDs of Comparative Examples 2 to 15 were produced in the same manner as in Device Examples 4 to 17, respectively, except that compound H-B was used instead of compound H2-2-D22 as the second host of the light-emitting layer.

The light-emitting color, and the time taken for luminance to decrease from 100% to 80% at a luminance of 60,000 nit (lifetime; T80) of the OLEDs produced in Device Examples 4 to 15 and Comparative Examples 2 to 13 are provided in Table 2 below.

TABLE 2

| | | | Light- | |
|---|---|---|---|---|
| | First | Second | Emitting | Lifetime |
| | Host | Host | Color | (T80) [hr] |
| Device | H1-271 | H2-2-D22 | Green | 1184 |
| Example 4 | | | | |
| Comparative | H1-271 | H-B | Green | 1054 |
| Example 2 | | | | |
| Device | H1-325 | H2-2-D22 | Green | 135 |
| Example 5 | | | | |
| Comparative | H1-325 | H-B | Green | 122 |
| Example 3 | | | | |
| Device | H1-326 | H2-2-D22 | Green | 263 |
| Example 6 | | | | |
| Comparative | H1-326 | H-B | Green | 212 |
| Example 4 | | | | |
| Device | H1-221 | H2-2-D22 | Green | 195 |
| Example 7 | | | | |
| Comparative | H1-221 | H-B | Green | 160 |
| Example 5 | | | | |
| Device | H1-57 | H2-2-D22 | Green | 113 |
| Example 8 | | | | |
| Comparative | H1-57 | H-B | Green | 97 |
| Example 6 | | | | |
| Device | H1-327 | H2-2-D22 | Green | 130 |
| Example 9 | | | | |
| Comparative | H1-327 | H-B | Green | 104 |
| Example 7 | | | | |
| Device | H1-324 | H2-2-D22 | Green | 62 |
| Example 10 | | | | |
| Comparative | H1-324 | H-B | Green | 56 |
| Example 8 | | | | |
| Device | H1-323 | H2-2-D22 | Green | 43 |
| Example 11 | | | | |
| Comparative | H1-323 | H-B | Green | 38 |
| Example 9 | | | | |
| Device | H1-328 | H2-2-D22 | Green | 95 |
| Example 12 | | | | |
| Comparative | H1-328 | H-B | Green | 85 |
| Example 10 | | | | |
| Device | H1-192 | H2-2-D22 | Green | 91 |
| Example 13 | | | | |
| Comparative | H1-192 | H-B | Green | 77 |
| Example 11 | | | | |
| Device | H1-277 | H2-2-D22 | Green | 112 |
| Example 14 | | | | |
| Comparative | H1-277 | H-B | Green | 97 |
| Example 12 | | | | |
| Device | H1-275 | H2-2-D22 | Green | 143 |
| Example 15 | | | | |
| Comparative | H1-275 | H-B | Green | 125 |
| Example 13 | | | | |

The light-emitting color, and the time taken for luminance to decrease from 100% to 80% at a luminance of 20,000 nit (lifetime; T80) of the OLEDs produced in Device Examples 16 and 17 and Comparative Examples 14 and 15 are provided in Table 3 below.

TABLE 3

| | | | Light- | |
|---|---|---|---|---|
| | First | Second | Emitting | Lifetime |
| | Host | Host | Color | (T80) [hr] |
| Device | H1-262 | H2-2-D22 | Green | 525 |
| Example 16 | | | | |
| Comparative | H1-262 | H-B | Green | 432 |
| Example 14 | | | | |
| Device | H1-281 | H2-2-D22 | Green | 894 |
| Example 17 | | | | |
| Comparative | H1-281 | H-B | Green | 790 |
| Example 15 | | | | |

From Tables 1 to 3 above, it can be seen that the OLEDs comprising a specific combination of compounds according to the present disclosure as host materials exhibit significantly improved lifetime properties, while having driving voltage and/or luminous efficiency in equivalent level, compared to the OLEDs comprising the conventional compounds.

In general, the green light-emitting organic electroluminescent device has shorter lifetime compared to the red light-emitting organic electroluminescent device. In order to improve the lifetime property of the green light-emitting organic electroluminescent device, the present disclosure used the compound having a deuterated moiety. Without limiting to only theory itself, if an organic electroluminescent compound is substituted with deuterium, it may lower the zero point vibration energy of the compound and increase the bond dissociation energy (BDE) in the compound to increase the stability of the compound. It is understood that the application of the deuterated moiety to a H-host may increase the stability of the H-host and reduce the deterioration of the host, thereby improving the lifetime properties. FIG. 1 illustrates a graph showing the increase in bond dissociation energy related to deuteration.

### Device Examples 18 to 20: Producing an OLED Comprising a Compound According to the Present Disclosure as a Single Host

An OLED was produced in the same manner as in Device Example 4, except that only the host shown in Tables 4 and 5 below was used as the host of the light-emitting layer.

### Comparative Examples 16 and 17: Producing an OLED Comprising a Comparative Compound as a Single Host

An OLED was produced in the same manner as in Device Example 18, except that only the host shown in Tables 4 and 5 below was used as the host of the light-emitting layer.

The light-emitting color, and the time taken for luminance to decrease from 100% to 80% at a luminance of 20,000 nit (lifetime; T80) of the OLEDs produced in Device Examples 18 and 19 and Comparative Example 16 are provided in Table 4 below.

TABLE 4

| | | Light- | |
|---|---|---|---|
| | | Emitting | Lifetime |
| | Host | Color | (T80) [hr] |
| Device | H1-271 | Green | 170 |
| Example 18 | | | |
| Device | H1-275 | Green | 131 |
| Example 19 | | | |
| Comparative | H1-192 | Green | 112 |
| Example 16 | | | |

The light-emitting color, and the time taken for luminance to decrease from 100% to 80% at a luminance of 20,000 nit (lifetime: T80) of the OLEDs produced in Device Example 20 and Comparative Example 17 are provided in Table 5 below.

TABLE 5

| | | Light- | |
|---|---|---|---|
| | | Emitting | Lifetime |
| | Host | Color | (T80) [hr] |
| Device | H1-281 | Green | 46 |
| Example 20 | | | |
| Comparative | H1-262 | Green | 15 |
| Example 17 | | | |

From Tables 4 and 5 above, it can be seen that the organic electroluminescent compounds according to the present disclosure exhibit excellent light-emitting properties compared to the conventional materials. In addition, the OLEDs using the compounds according to the present disclosure as host materials exhibit excellent light-emitting properties as well as improved lifetime properties.

### Device Examples 21 and 22: Producing an OLED Comprising a Compound According to the Present Disclosure as a Single Host

OLEDs were produced in the same manner as in Device Example 1, except that the second hole transport layer, the light-emitting layer, and the electron transport layer were formed as follows: Compound HT-4 was introduced into another cell of the vacuum vapor deposition apparatus and was evaporated by applying an electric current to the cell, thereby forming a second hole transport layer having a thickness of 60 nm on the first hole transport layer. The host compound shown in Table 6 below was introduced into a cell of the vacuum vapor deposition apparatus as a host, and compound D-39 was introduced into another cell as a dopant. The host material and the dopant material were evaporated at different rates, and the dopant was deposited in a doping amount of 3 wt % based on the total amount of the host and the dopant to form a light-emitting layer having a thickness of 40 nm on the second hole transport layer. Compound ET-1 and compound EI-1 as electron transport materials were evaporated in a weight ratio of 50:50 to form an electron transport layer having a thickness of 35 nm on the light-emitting layer.

### Comparative Example 18: Producing an OLED Comprising a Comparative Compound as a Single Host

An OLED was produced in the same manner as in Device Example 21, except that only the host compound shown in Table 6 below was used as a single host of the light-emitting layer.

The driving voltage, luminous efficiency, and light-emitting color at a luminance of 1,000 nit, and the time taken for luminance to decrease from 100% to 90% at a luminance of 10,000 nit (lifetime; T90) of the OLEDs produced in Device Examples 21 and 22 and Comparative Example 18 are provided in Table 6 below.

TABLE 6

| | | Driving | Luminous | Light- | |
|---|---|---|---|---|---|
| | | Voltage | Efficiency | Emitting | Lifetime |
| | Host | [V] | [cd/A] | Color | T90 [hr] |
| Device Example | H1-343 | 3.8 | 31.7 | Red | 23 |
| 21 | | | | | |
| Device Example | H1-356 | 3.2 | 29.3 | Red | 27 |
| 22 | | | | | |
| Comparative | H1-B | 3.3 | 28.5 | Red | 17 |
| Example 18 | | | | | |

### Device Examples 23 and 24: Producing an OLED Co-Deposited with the First Host Compound and the Second Host Compound According to the Present Disclosure

OLEDs were produced in the same manner as in Device Example 21, except that the light-emitting layer was formed as follows: The first host compound shown in Table 7 below and compound H2-A (the second host) were introduced into two cells of the vacuum vapor deposition apparatus as hosts, and compound D-39 was introduced into another cell as a dopant. The two host materials were evaporated at a rate of 1:1, and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer.

### Comparative Examples 19 and 20: Producing an OLED Comprising a Comparative Compound as the First Host

OLEDs were produced in the same manner as in Device Example 23, except that the compound shown in Table 7 below was used as the first host of the light-emitting layer.

The driving voltage, luminous efficiency, and light-emitting color at a luminance of 1,000 nit, and the time taken for luminance to decrease from 100% to 80% at a luminance of 10,000 nit (lifetime; T80) of the OLEDs produced in Device Examples 23 and 24 and Comparative Examples 19 and 20 are provided in Table 7 below.

TABLE 7

| | | Driving | Luminous | Light- | |
|---|---|---|---|---|---|
| | First | Voltage | Efficiency | Emitting | Lifetime |
| | Host | [V] | [cd/A] | Color | T90 [hr] |
| Device Example | H1-343 | 3.1 | 37.8 | Red | 938 |
| 23 | | | | | |
| Device Example | H1-356 | 3.0 | 36.7 | Red | 932 |
| 24 | | | | | |
| Comparative | H1-A | 3.2 | 37.6 | Red | 834 |
| Example 19 | | | | | |
| Comparative | H1-B | 3.0 | 36.4 | Red | 633 |
| Example 20 | | | | | |

From Tables 6 and 7 above, it can be confirmed that the organic electroluminescent compounds according to the present disclosure exhibit excellent light-emitting properties compared to the conventional materials. In addition, it can be seen that the OLEDs comprising the compound according to the present disclosure as a single host or as one of a plurality of hosts exhibit significantly improved lifetime properties, while having driving voltage and/or luminous efficiency in equivalent level, compared to the OLEDs comprising the conventional compounds.

The compounds used in the Device Examples and the Comparative Example are shown in Table 8 below.

TABLE 8

| | |
|---|---|
| Hole Injection Layer/ Hole Transport Layer | 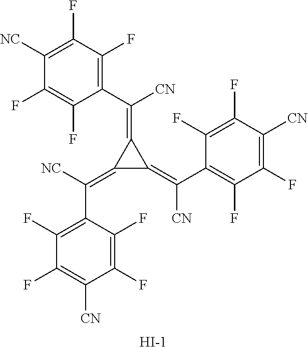 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 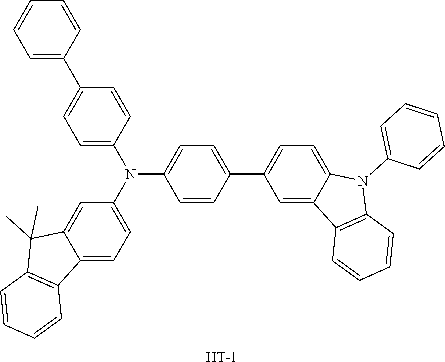 |
| | 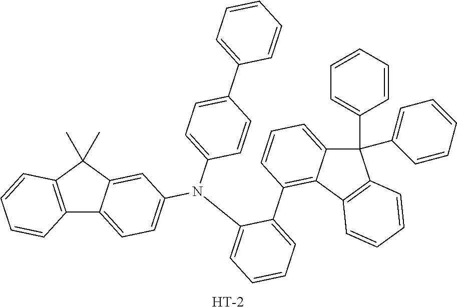 |
| | 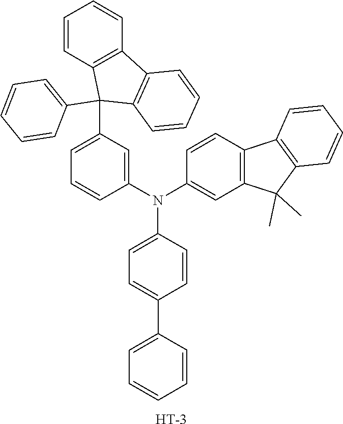 |
| | 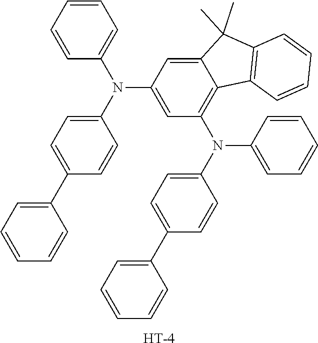 |
| Light- Emitting Layer | 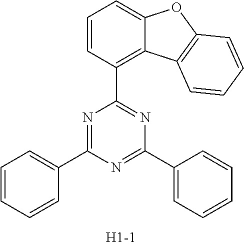 [SMILES: c1ccc(-c2nc(-c3ccccc3)nc(-c3cccc4oc5ccccc5c34)n2)cc1] |
| | 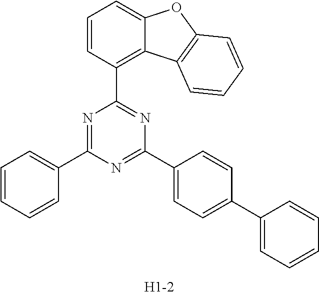 [SMILES: c1ccc(-c2ccc(-c3nc(-c4ccccc4)nc(-c4cccc5oc6ccccc6c45)n3)cc2)cc1] |
| | 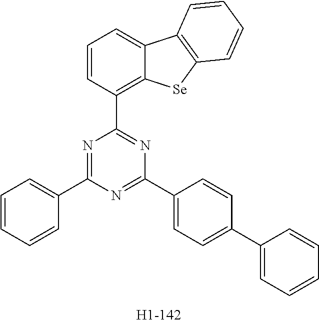 [SMILES: c1ccc(-c2ccc(-c3nc(-c4ccccc4)nc(-c4cccc5c4[se]c4ccccc45)n3)cc2)cc1] |
| | 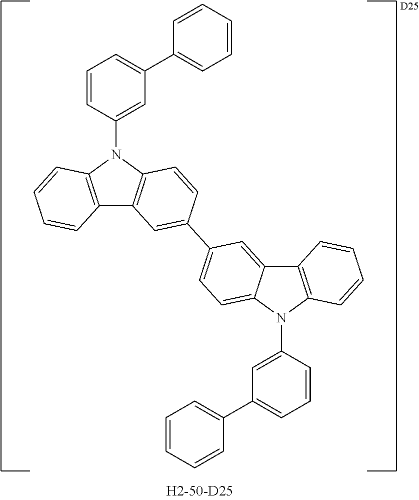 |
| | 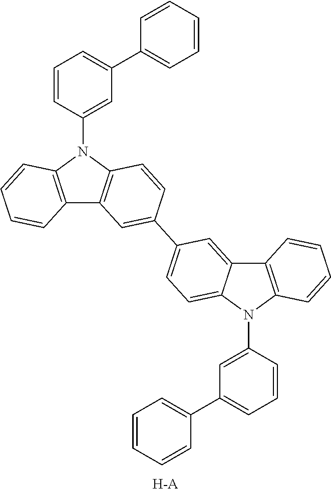 |
| | 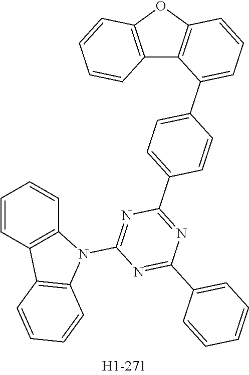 [SMILES: c1ccc(-c2nc(-c3ccc(-c4cccc5oc6ccccc6c45)cc3)nc(-n3c4ccccc4c4ccccc43)n2)cc1] |
| | 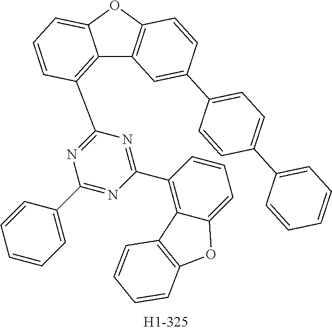 [SMILES: c1ccc(-c2ccc(-c3ccc4oc5cccc(-c6nc(-c7ccccc7)nc(-c7cccc8oc9ccccc9c78)n6)c5c4c3)cc2)cc1] |
| | 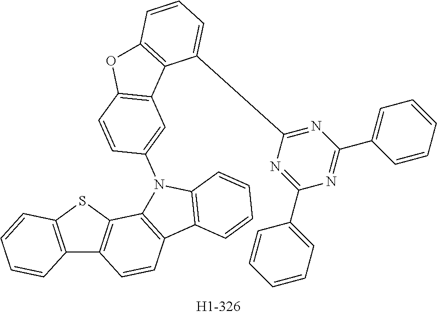 [SMILES: c1ccc(-c2nc(-c3ccccc3)nc(-c3cccc4oc5ccc(-n6c7ccccc7c7ccc8c9ccccc9sc8c76)cc5c34)n2)cc1] |
| | 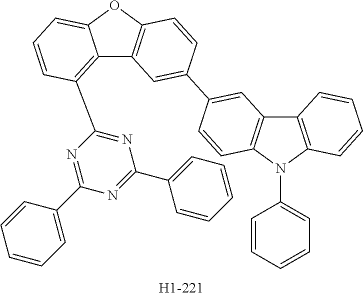 [SMILES: c1ccc(-c2nc(-c3ccccc3)nc(-c3cccc4oc5ccc(-c6ccc7c(c6)c6ccccc6n7-c6ccccc6)cc5c34)n2)cc1] |
| | 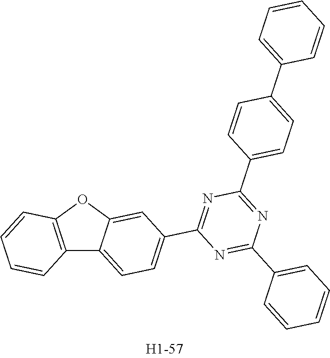 [SMILES: c1ccc(-c2ccc(-c3nc(-c4ccccc4)nc(-c4ccc5c(c4)oc4ccccc45)n3)cc2)cc1] |
| | 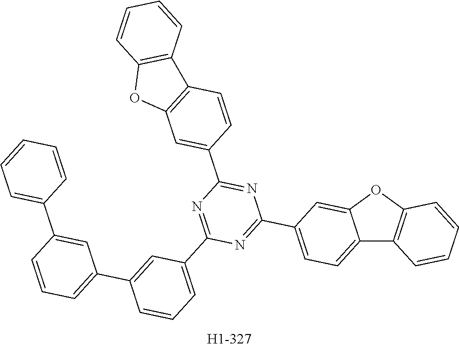 [SMILES: c1ccc(-c2cccc(-c3cccc(-c4nc(-c5ccc6c(c5)oc5ccccc56)nc(-c5ccc6c(c5)oc5ccccc56)n4)c3)c2)cc1] |
| | 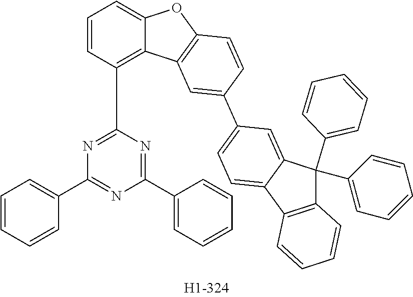 [SMILES: c1ccc(-c2nc(-c3ccccc3)nc(-c3cccc4oc5ccc(-c6ccc7c(c6)C(c6ccccc6)(c6ccccc6)c6ccccc6-7)cc5c34)n2)cc1] |
| | 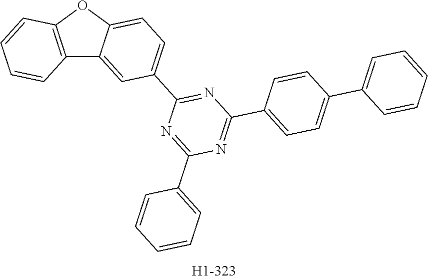 [SMILES: c1ccc(-c2ccc(-c3nc(-c4ccccc4)nc(-c4ccc5oc6ccccc6c5c4)n3)cc2)cc1] |
| | 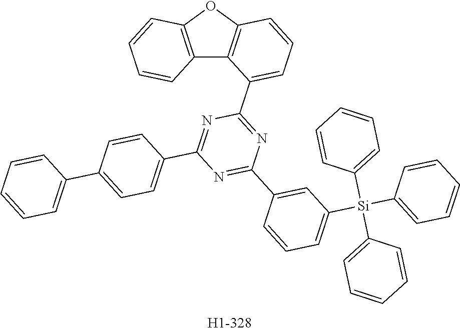 [SMILES: c1ccc(-c2ccc(-c3nc(-c4cccc([Si](c5ccccc5)(c5ccccc5)c5ccccc5)c4)nc(-c4cccc5oc6ccccc6c45)n3)cc2)cc1] |
| | 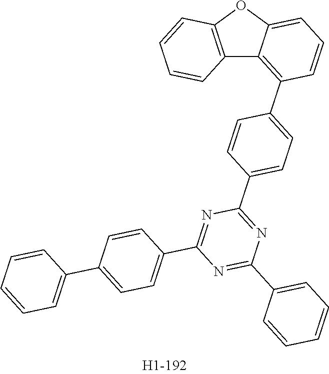 [SMILES: c1ccc(-c2ccc(-c3nc(-c4ccccc4)nc(-c4ccc(-c5cccc6oc7ccccc7c56)cc4)n3)cc2)cc1] |
| | 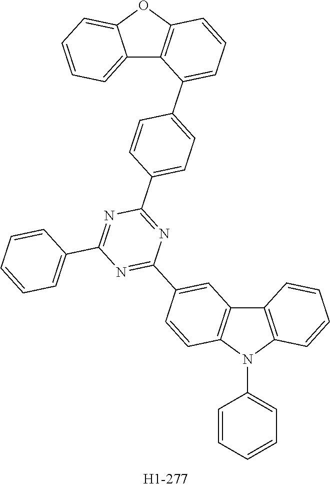 [SMILES: c1ccc(-c2nc(-c3ccc(-c4cccc5oc6ccccc6c45)cc3)nc(-c3ccc4c(c3)c3ccccc3n4-c3ccccc3)n2)cc1] |
| | 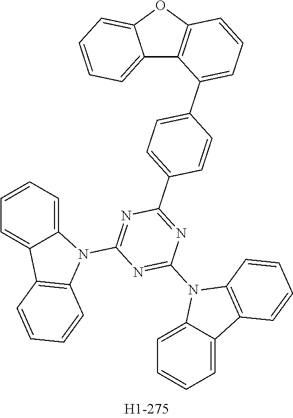 [SMILES: c1ccc2c(c1)oc1cccc(-c3ccc(-c4nc(-n5c6ccccc6c6ccccc65)nc(-n5c6ccccc6c6ccccc65)n4)cc3)c12] |
| | 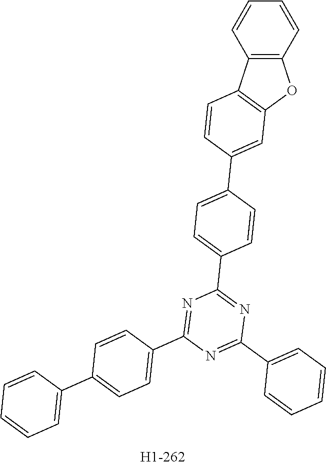 [SMILES: c1ccc(-c2ccc(-c3nc(-c4ccccc4)nc(-c4ccc(-c5ccc6c(c5)oc5ccccc56)cc4)n3)cc2)cc1] |
| | 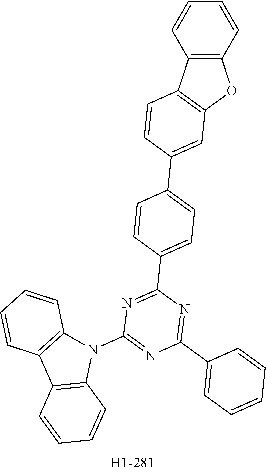 [SMILES: c1ccc(-c2nc(-c3ccc(-c4ccc5c(c4)oc4ccccc45)cc3)nc(-n3c4ccccc4c4ccccc43)n2)cc1] |
| | 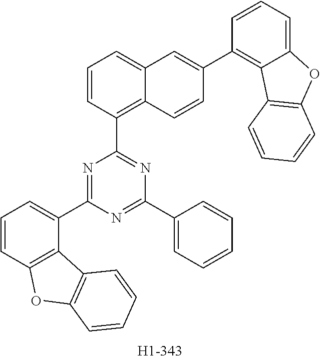 [SMILES: c1ccc(-c2nc(-c3cccc4cc(-c5cccc6oc7ccccc7c56)ccc34)nc(-c3cccc4oc5ccccc5c34)n2)cc1] |
| | 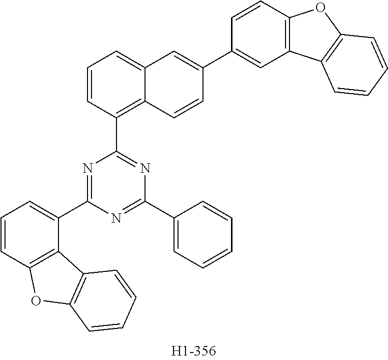 [SMILES: c1ccc(-c2nc(-c3cccc4cc(-c5ccc6oc7ccccc7c6c5)ccc34)nc(-c3cccc4oc5ccccc5c34)n2)cc1] |
| | 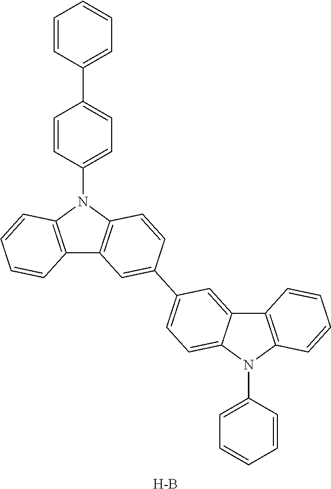 [SMILES: c1ccc(-c2ccc(-n3c4ccccc4c4cc(-c5ccc6c(c5)c5ccccc5n6-c5ccccc5)ccc43)cc2)cc1 ; "H-B"] |
| | 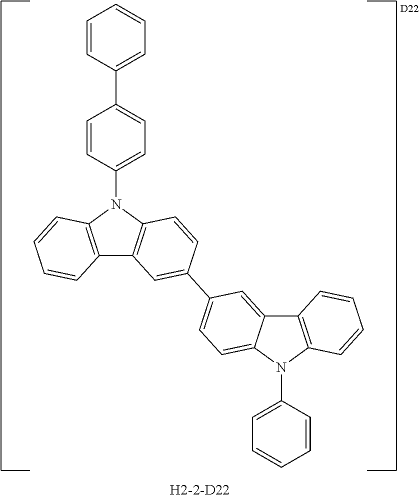 |
| | 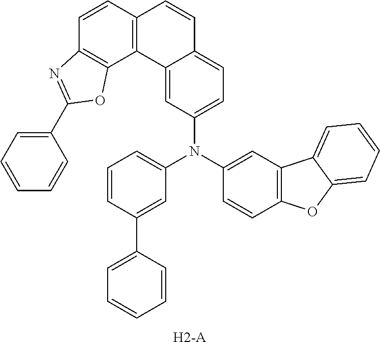 |
| | 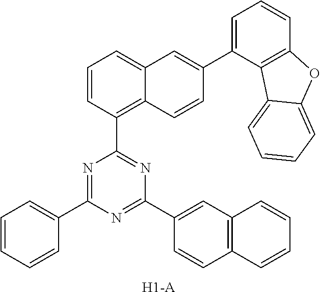 |
| | 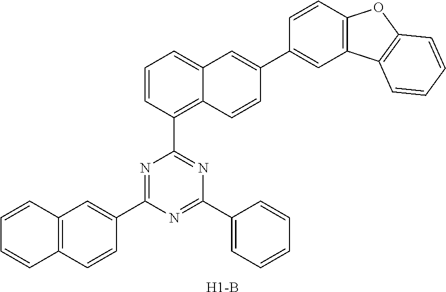 |
| | 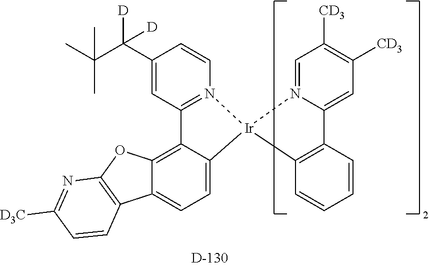 |
| | 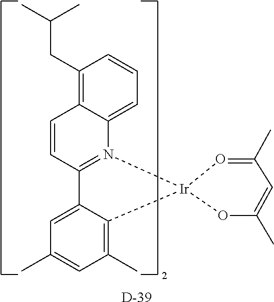 |
| Electron Transport Layer/ Electron Injection Layer | 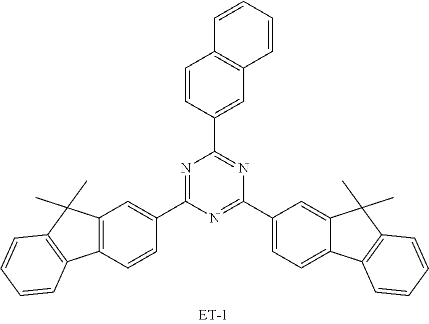 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 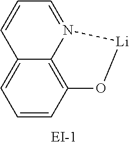 |

1. A plurality of host materials comprising a first host material(s) comprising the compound represented by the following formula 1, and a second host material(s) comprising the compound represented by the following formula 2: 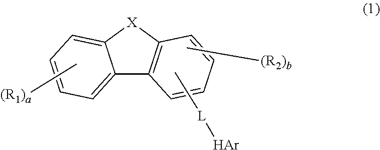
in formula 1,
X represents O, S or Se;
HAr represents a substituted or unsubstituted (3- to 30-membered)heteroaryl containing at least one nitrogen atom;
L represents a single bond, a substituted or unsubstituted (C6-C30)arylene, or a substituted or unsubstituted (3- to 30-membered)heteroarylene;
R₁ and R₂, each independently, represent hydrogen, deuterium, a substituted or unsubstituted (C6-C30)aryl, a substituted or unsubstituted (3- to 30-membered)heteroaryl, or —SiR₁₁R₁₂R₁₃;
R₁₁ to R₁₃, each independently, represent a substituted or unsubstituted (C1-C30)alkyl, a substituted or unsubstituted (C6-C30)aryl, or a substituted or unsubstituted (3- to 30-membered)heteroaryl; and
a represents an integer of 1 to 4, and b represents an integer of 1 to 3, in which if each of a and b is an integer of 2 or more, each of R₁ and each of R₂ may be the same or different; 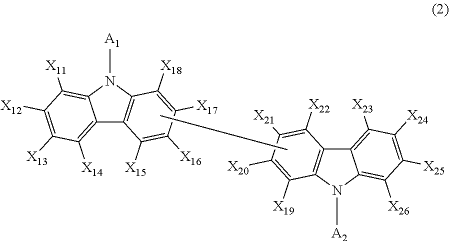
in formula 2,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;
X₁₁ to X₂₆, each independently, represent hydrogen, deuterium, a substituted or unsubstituted (C6-C30)aryl, or a substituted or unsubstituted (3- to 30-membered)heteroaryl; and
one of X₁₅ to X₁₈ and one of X₁₉ to X₂₂ are linked to each other to form a single bond,
with the proviso that at least one of X₁₁, X₁₈, X₁₉ and X₂₆ is deuterium.

2. The plurality of host materials according to claim 1, wherein HAr is a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzothienopyrimidinyl, a substituted or unsubstituted carbazolyl, or a substituted or unsubstituted pyridopyrazinyl.

3. The plurality of host materials according to claim 1, wherein the formula 1 is represented by the following formula 1-1: 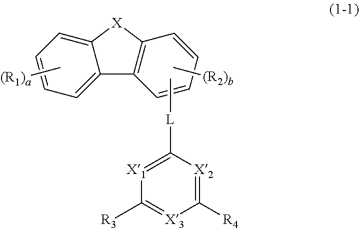
in formula 1-1,
X, R₁, R₂, L, a, and b are as defined in claim 1,
X′₁ to X′₃, each independently, represent CR′ or N, in which R′ represents hydrogen or deuterium, and at least two of X′₁ to X′₃ represent N; and
R₃ and R₄, each independently, represent a substituted or unsubstituted (C6-C30)aryl, or a substituted or unsubstituted (3- to 30-membered)heteroaryl.

4. The plurality of host materials according to claim 3, wherein the formula 1-1 is represented by any one of the following formulas 1-1-1 to 1-1-4: 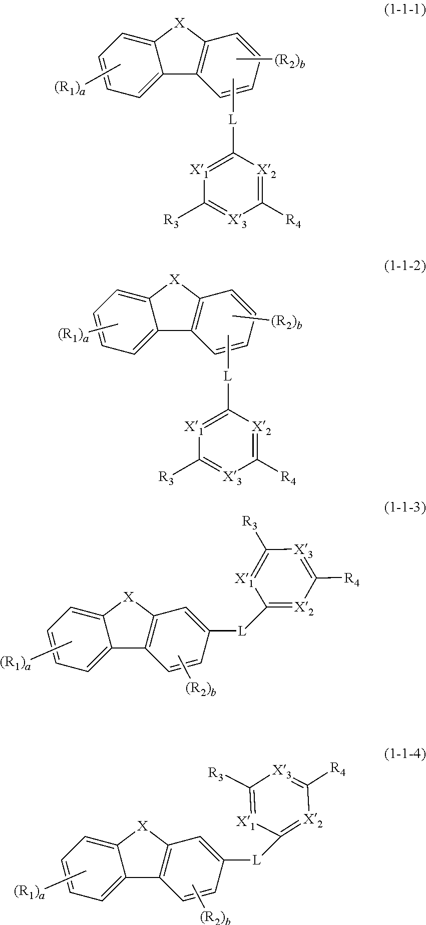
in formulas 1-1-1 to 1-1-4,
X, X′₁ to X′₃, R₁ to R₄, L, a, and b are as defined in claim 3.

5. The plurality of host materials according to claim 1, wherein the formula 2 is represented by any one of the following formulas 2-1 to 2-8: 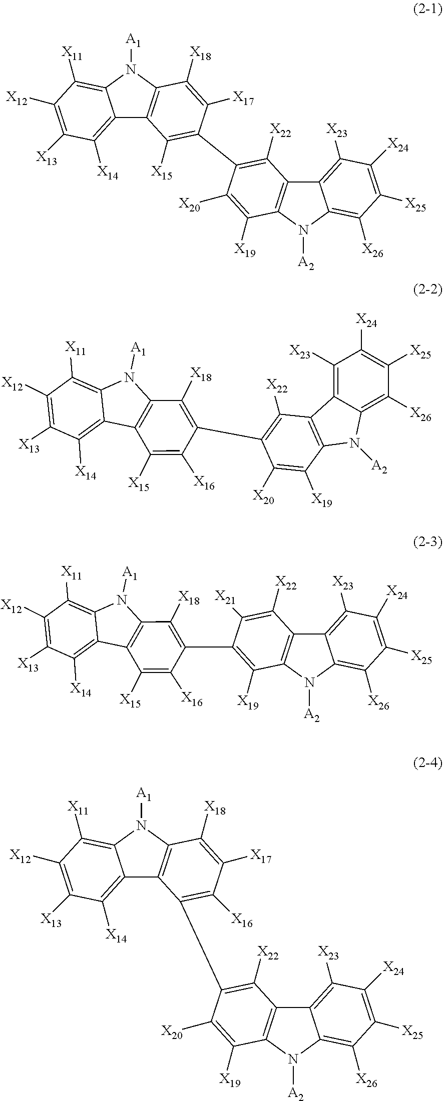 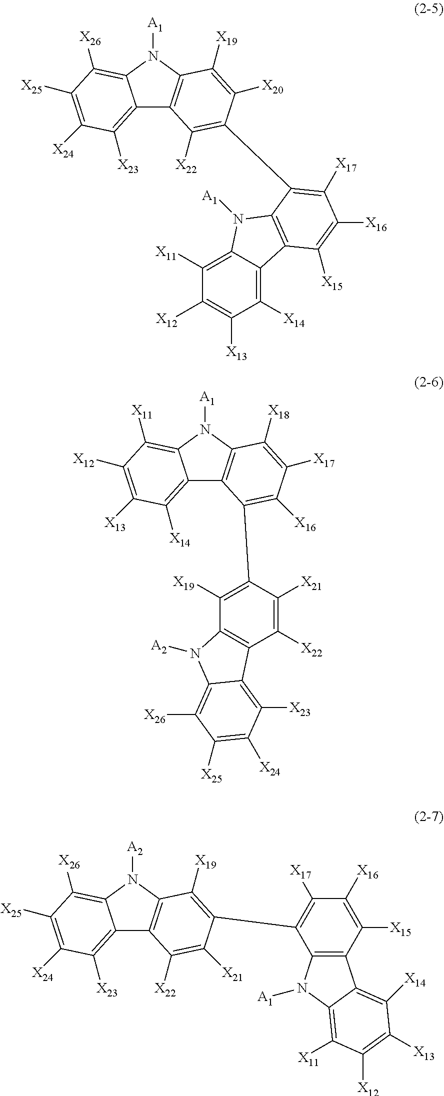 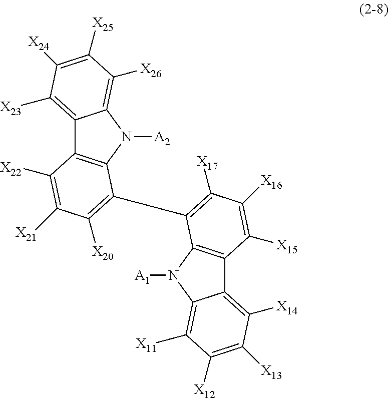
in formulas 2-1 to 2-8,
A₁, A₂, and X₁₁ to X₂₆ are as defined in claim 1.

6. The plurality of host materials according to claim 1, wherein A₁ and A₂ in formula 2, each independently, are a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl.

7. The plurality of host materials according to claim 1, wherein the compound represented by formula 1 is at least one selected from the group consisting of the following compounds:

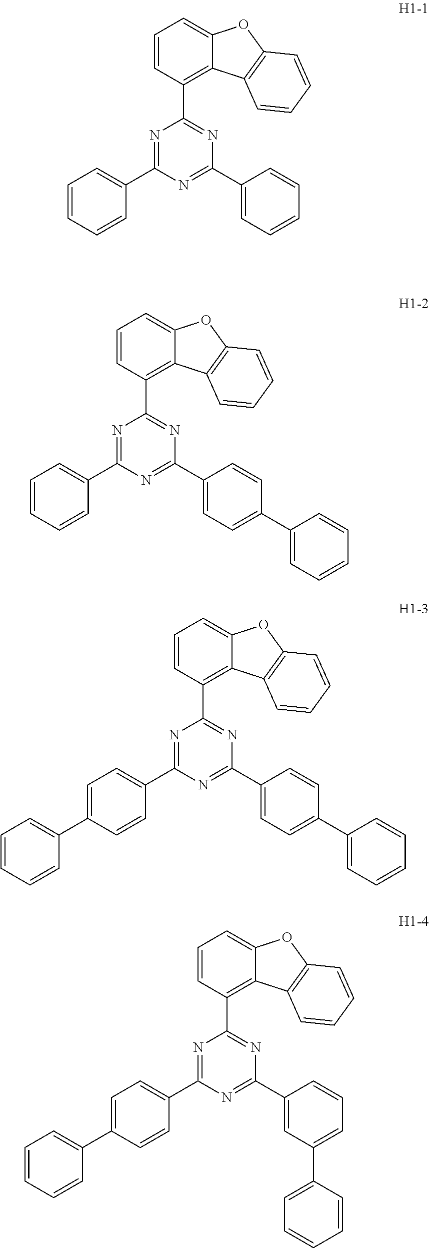 [SMILES: c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1]

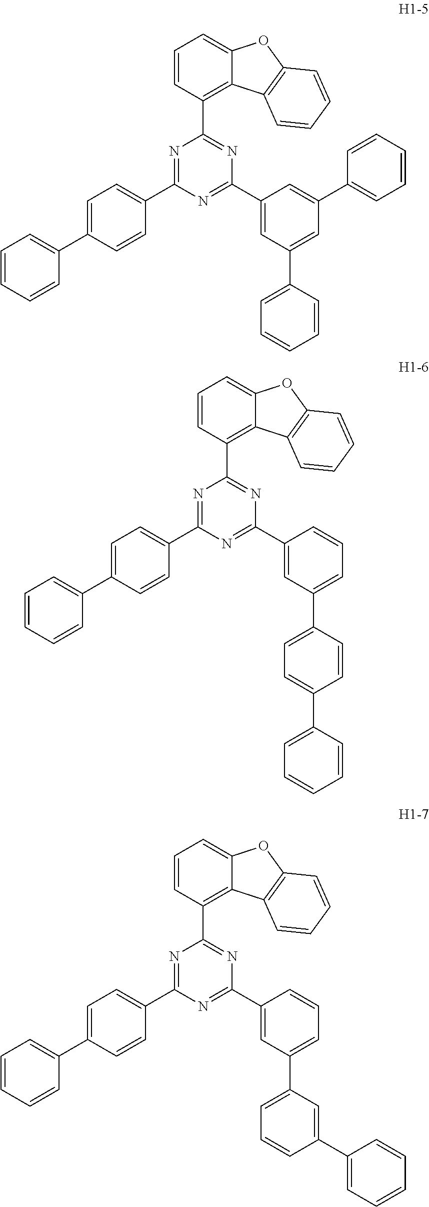

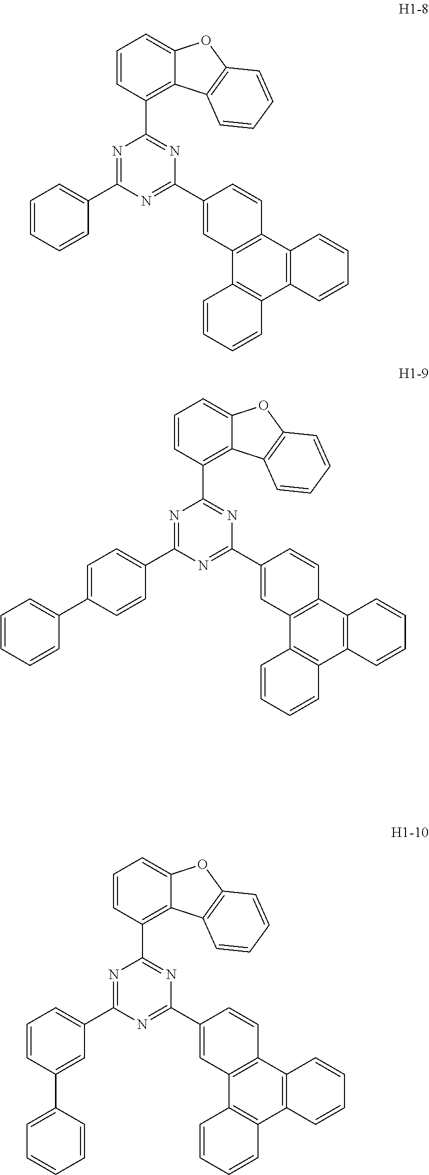 [SMILES: c1ccc(-c2nc(-c3ccc4c5ccccc5c5ccccc5c4c3)nc(-c3cccc4oc5ccccc5c34)n2)cc1 ; c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1]

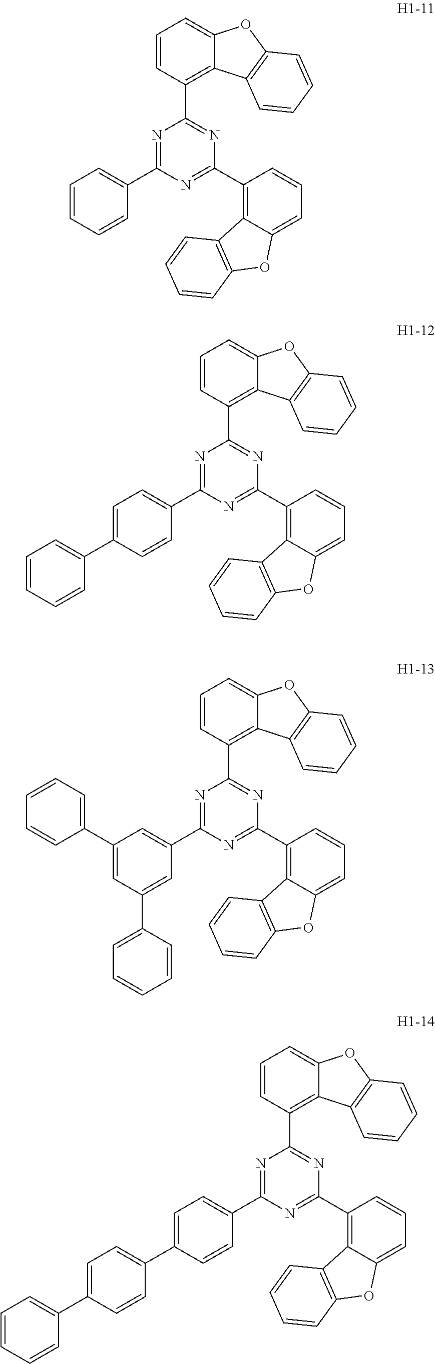

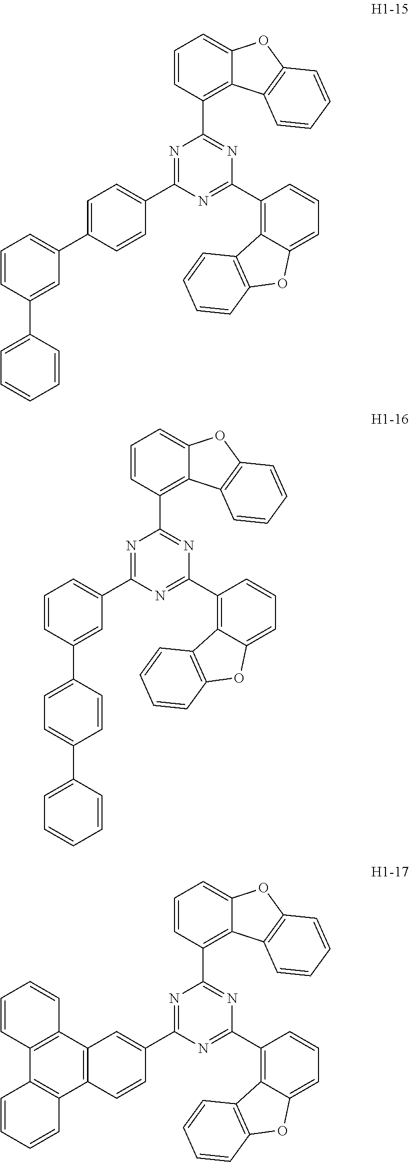 [SMILES: c1ccc(-c2cccc(-c3ccc(-c4nc(-c5cccc6oc7ccccc7c56)nc(-c5cccc6oc7ccccc7c56)n4)cc3)c2)cc1 ; c1ccc(-c2ccc(-c3cccc(-c4nc(-c5cccc6oc7ccccc7c56)nc(-c5cccc6oc7ccccc7c56)n4)c3)cc2)cc1 ; c1ccc2c(c1)oc1cccc(-c3nc(-c4ccc5c6ccccc6c6ccccc6c5c4)nc(-c4cccc5oc6ccccc6c45)n3)c12]

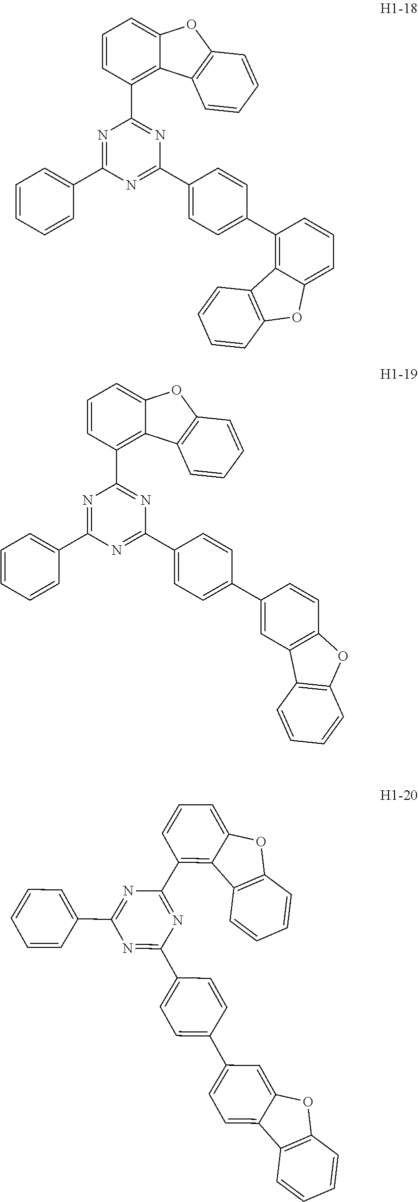 [SMILES: c1ccc(-c2nc(-c3ccc(-c4cccc5oc6ccccc6c45)cc3)nc(-c3cccc4oc5ccccc5c34)n2)cc1 ; c1ccc(-c2nc(-c3ccc(-c4ccc5oc6ccccc6c5c4)cc3)nc(-c3cccc4oc5ccccc5c34)n2)cc1 ; c1ccc(-c2nc(-c3ccc(-c4ccc5c(c4)oc4ccccc45)cc3)nc(-c3cccc4oc5ccccc5c34)n2)cc1]

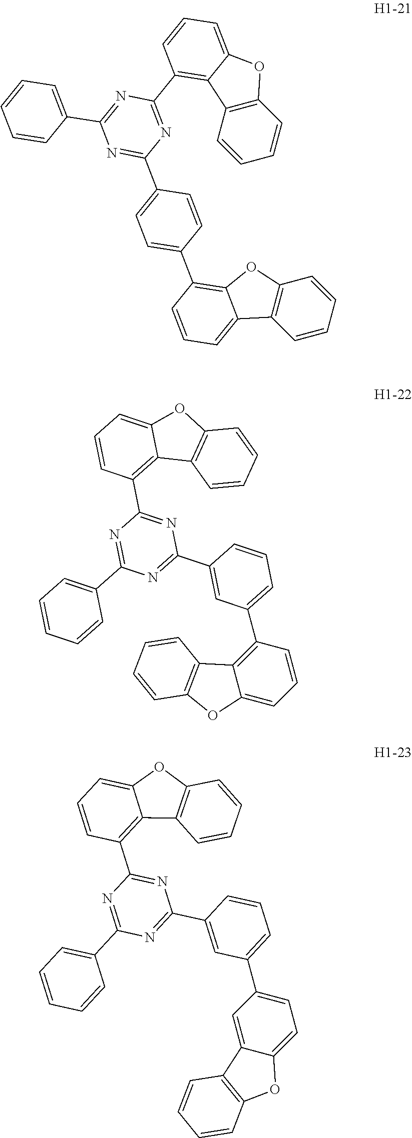 [SMILES: c1ccc(-c2nc(-c3ccc(-c4cccc5c4oc4ccccc45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3cccc(-c4ccc5oc6ccccc6c5c4)c3)nc(-c3cccc4oc5ccccc5c34)n2)cc1]

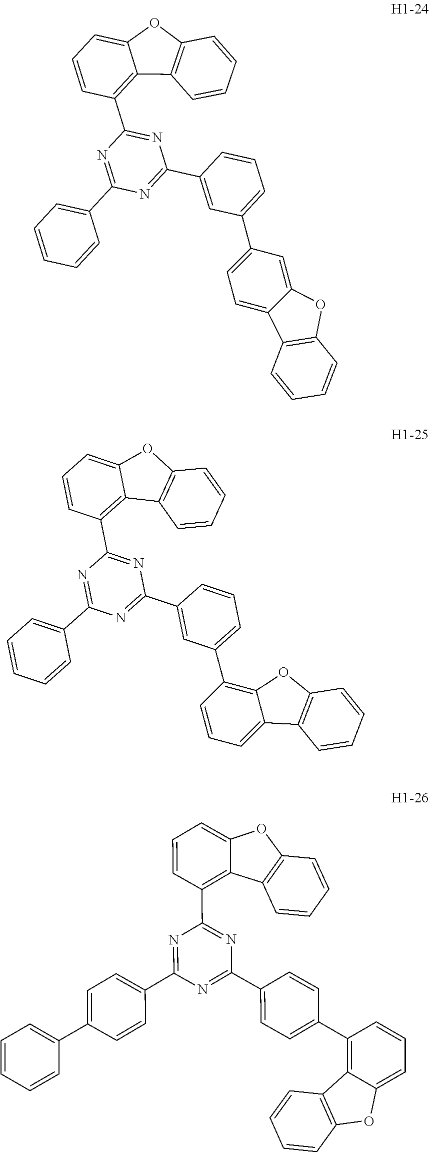 [SMILES: c1ccc(-c2nc(-c3cccc(-c4ccc5c(c4)oc4ccccc45)c3)nc(-c3cccc4oc5ccccc5c34)n2)cc1 ; c1ccc(-c2nc(-c3cccc(-c4cccc5c4oc4ccccc45)c3)nc(-c3cccc4oc5ccccc5c34)n2)cc1 ; c1ccc(-c2ccc(-c3nc(-c4ccc(-c5cccc6oc7ccccc7c56)cc4)nc(-c4cccc5oc6ccccc6c45)n3)cc2)cc1]

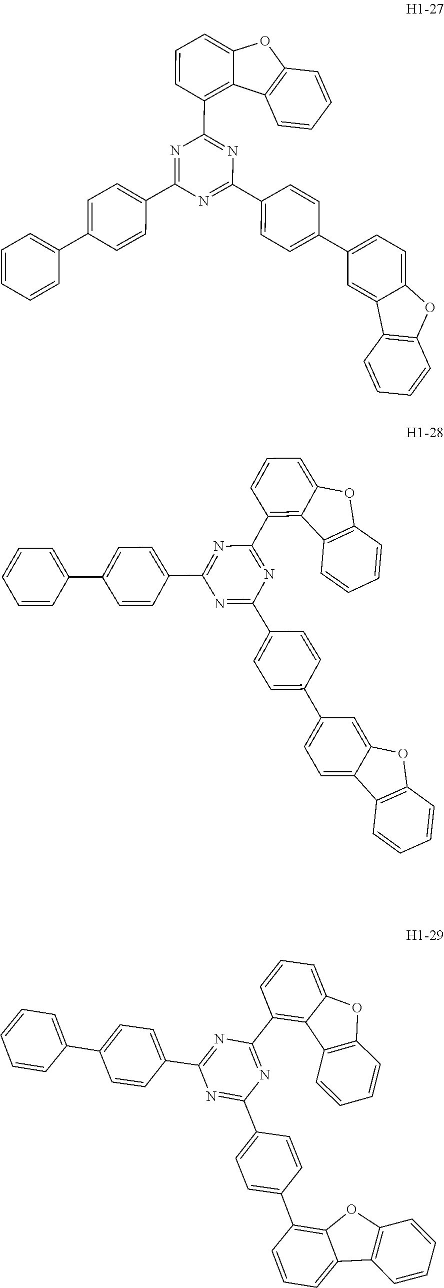 [SMILES: c1ccc(-c2ccc(-c3nc(-c4ccc(-c5ccc6oc7ccccc7c6c5)cc4)nc(-c4cccc5oc6ccccc6c45)n3)cc2)cc1 ; c1ccc(-c2ccc(-c3nc(-c4ccc(-c5ccc6c(c5)oc5ccccc56)cc4)nc(-c4cccc5oc6ccccc6c45)n3)cc2)cc1 ; c1ccc(-c2ccc(-c3nc(-c4ccc(-c5cccc6c5oc5ccccc56)cc4)nc(-c4cccc5oc6ccccc6c45)n3)cc2)cc1]

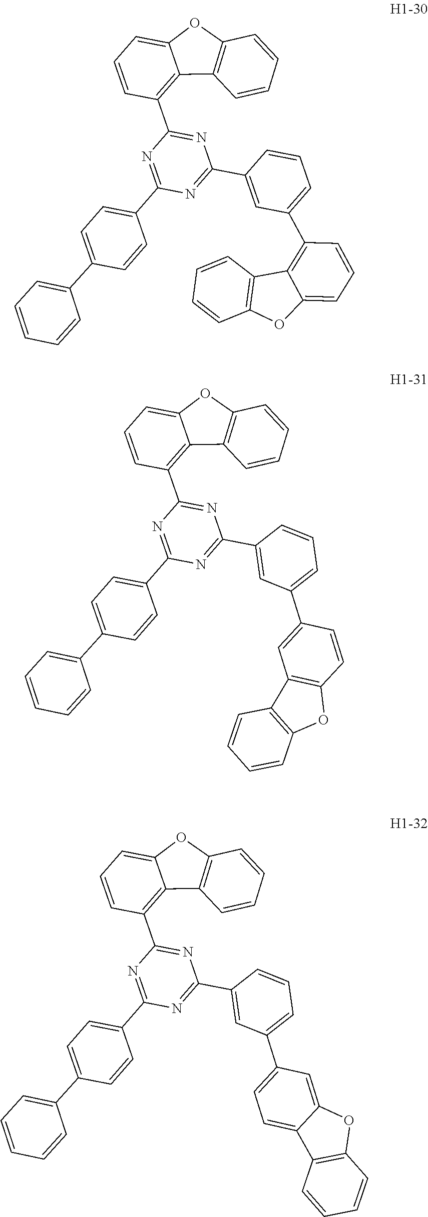 [SMILES: c1ccc(-c2ccc(-c3nc(-c4cccc(-c5cccc6oc7ccccc7c56)c4)nc(-c4cccc5oc6ccccc6c45)n3)cc2)cc1 ; c1ccc(-c2ccc(-c3nc(-c4cccc(-c5ccc6oc7ccccc7c6c5)c4)nc(-c4cccc5oc6ccccc6c45)n3)cc2)cc1 ; c1ccc(-c2ccc(-c3nc(-c4cccc(-c5ccc6c(c5)oc5ccccc56)c4)nc(-c4cccc5oc6ccccc6c45)n3)cc2)cc1]

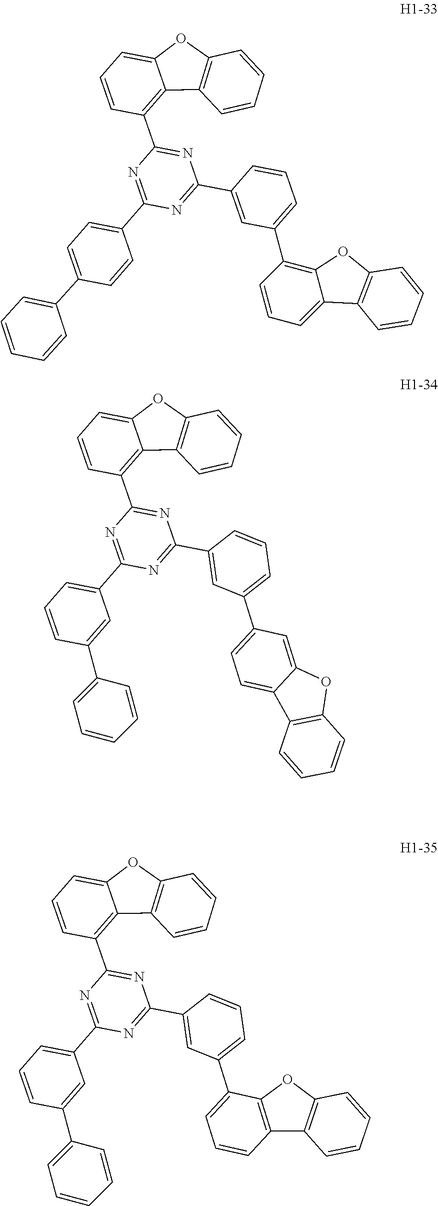 [SMILES: c1ccc(-c2ccc(-c3nc(-c4cccc(-c5cccc6c5oc5ccccc56)c4)nc(-c4cccc5oc6ccccc6c45)n3)cc2)cc1 ; c1ccc(-c2cccc(-c3nc(-c4cccc(-c5ccc6c(c5)oc5ccccc56)c4)nc(-c4cccc5oc6ccccc6c45)n3)c2)cc1 ; c1ccc(-c2cccc(-c3nc(-c4cccc(-c5cccc6c5oc5ccccc56)c4)nc(-c4cccc5oc6ccccc6c45)n3)c2)cc1]

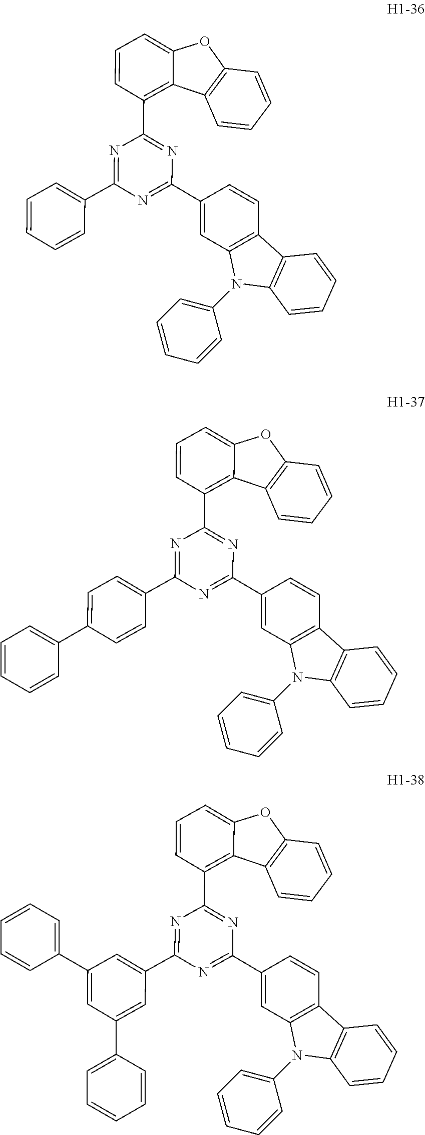 [SMILES: c1ccc(-c2nc(-c3ccc4c5ccccc5n(-c5ccccc5)c4c3)nc(-c3cccc4oc5ccccc5c34)n2)cc1 ; c1ccc(-c2ccc(-c3nc(-c4ccc5c6ccccc6n(-c6ccccc6)c5c4)nc(-c4cccc5oc6ccccc6c45)n3)cc2)cc1 ; c1ccc(-c2cc(-c3ccccc3)cc(-c3nc(-c4ccc5c6ccccc6n(-c6ccccc6)c5c4)nc(-c4cccc5oc6ccccc6c45)n3)c2)cc1]

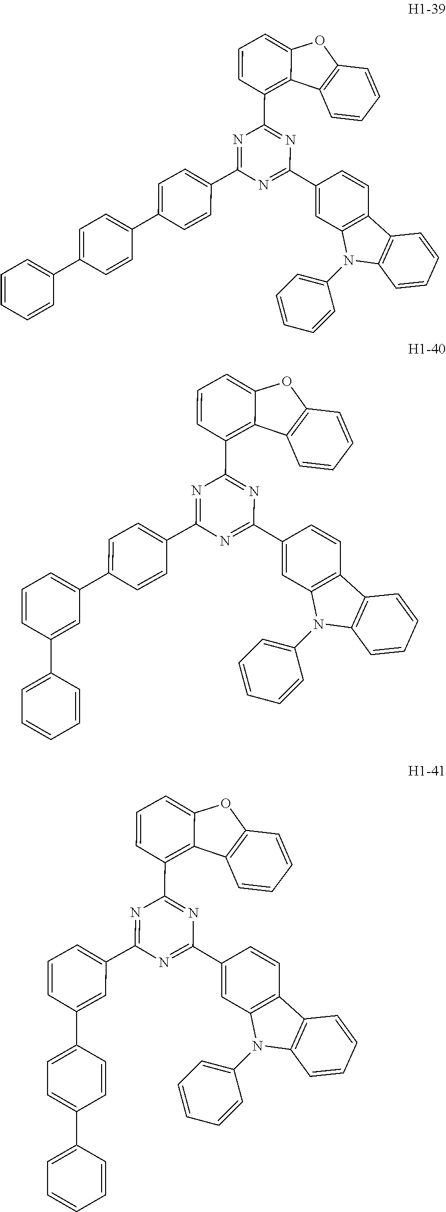 [SMILES: c1ccc(-c2ccc(-c3ccc(-c4nc(-c5ccc6c7ccccc7n(-c7ccccc7)c6c5)nc(-c5cccc6oc7ccccc7c56)n4)cc3)cc2)cc1 ; c1ccc(-c2cccc(-c3ccc(-c4nc(-c5ccc6c7ccccc7n(-c7ccccc7)c6c5)nc(-c5cccc6oc7ccccc7c56)n4)cc3)c2)cc1 ; c1ccc(-c2ccc(-c3cccc(-c4nc(-c5ccc6c7ccccc7n(-c7ccccc7)c6c5)nc(-c5cccc6oc7ccccc7c56)n4)c3)cc2)cc1]

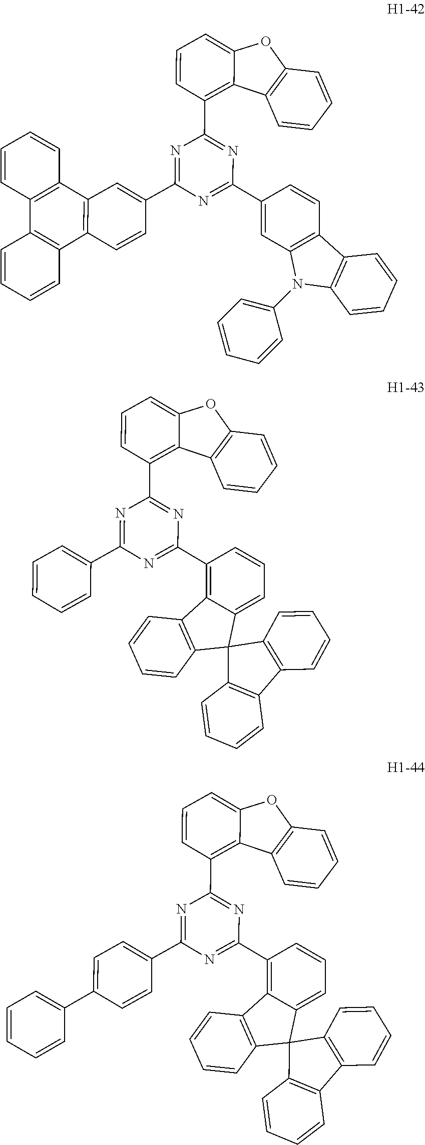 [SMILES: c1ccc(-n2c3ccccc3c3ccc(-c4nc(-c5ccc6c7ccccc7c7ccccc7c6c5)nc(-c5cccc6oc7ccccc7c56)n4)cc32)cc1 ; c1ccc(-c2nc(-c3cccc4c3-c3ccccc3C43c4ccccc4-c4ccccc43)nc(-c3cccc4oc5ccccc5c34)n2)cc1 ; c1ccc(-c2ccc(-c3nc(-c4cccc5c4-c4ccccc4C54c5ccccc5-c5ccccc54)nc(-c4cccc5oc6ccccc6c45)n3)cc2)cc1]

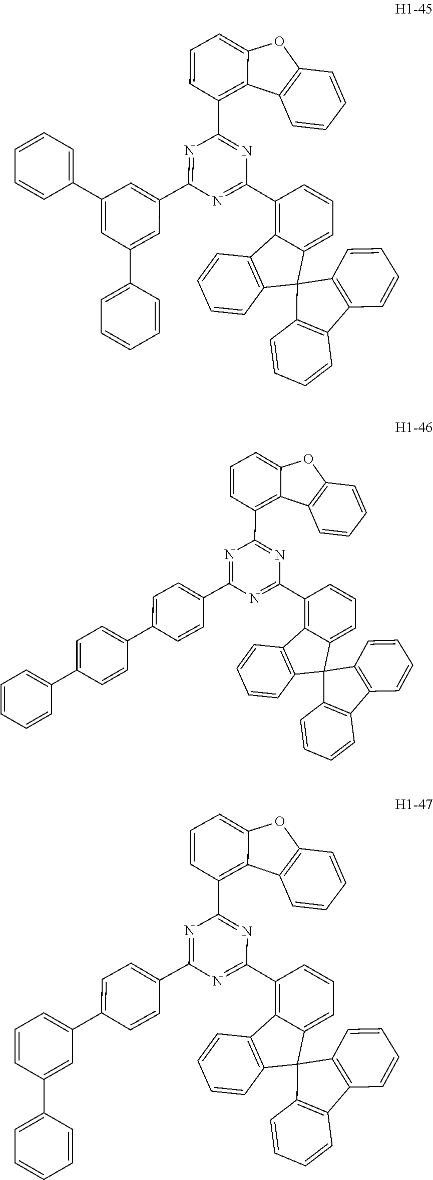 [SMILES: c1ccc(-c2cc(-c3ccccc3)cc(-c3nc(-c4cccc5c4-c4ccccc4C54c5ccccc5-c5ccccc54)nc(-c4cccc5oc6ccccc6c45)n3)c2)cc1 ; c1ccc(-c2ccc(-c3ccc(-c4nc(-c5cccc6c5-c5ccccc5C65c6ccccc6-c6ccccc65)nc(-c5cccc6oc7ccccc7c56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6oc7ccccc7c56)n4)cc3)c2)cc1]

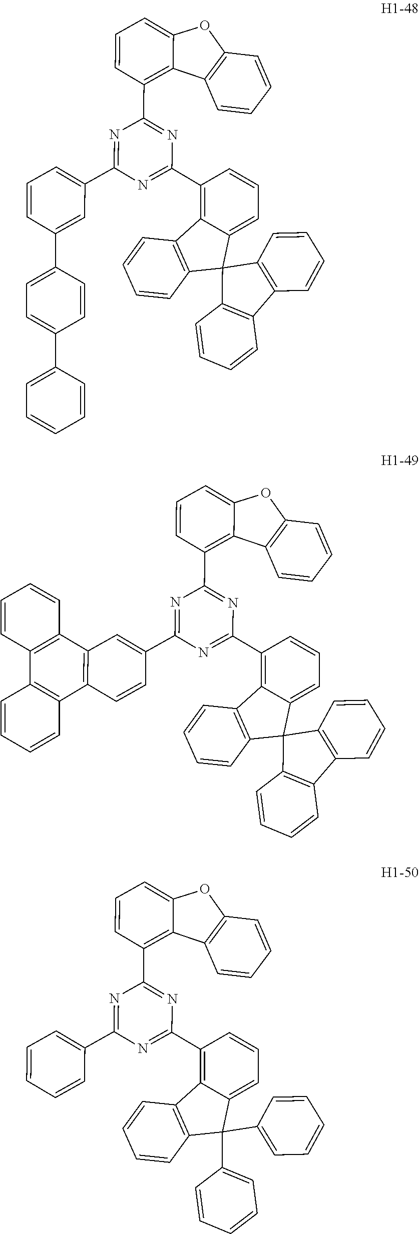 [SMILES: c1ccc(-c2ccc(-c3cccc(-c4nc(-c5cccc6c5-c5ccccc5C65c6ccccc6-c6ccccc65)nc(-c5cccc6oc7ccccc7c56)n4)c3)cc2)cc1 ; c1ccc2c(c1)-c1ccccc1C21c2ccccc2-c2c(-c3nc(-c4ccc5c6ccccc6c6ccccc6c5c4)nc(-c4cccc5oc6ccccc6c45)n3)cccc21 ; c1ccc(-c2nc(-c3cccc4c3-c3ccccc3C4(c3ccccc3)c3ccccc3)nc(-c3cccc4oc5ccccc5c34)n2)cc1]

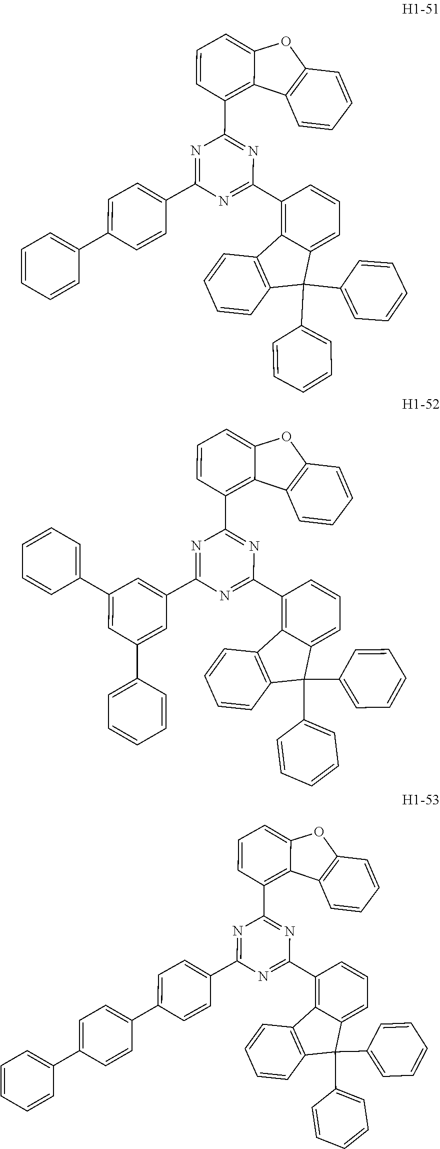

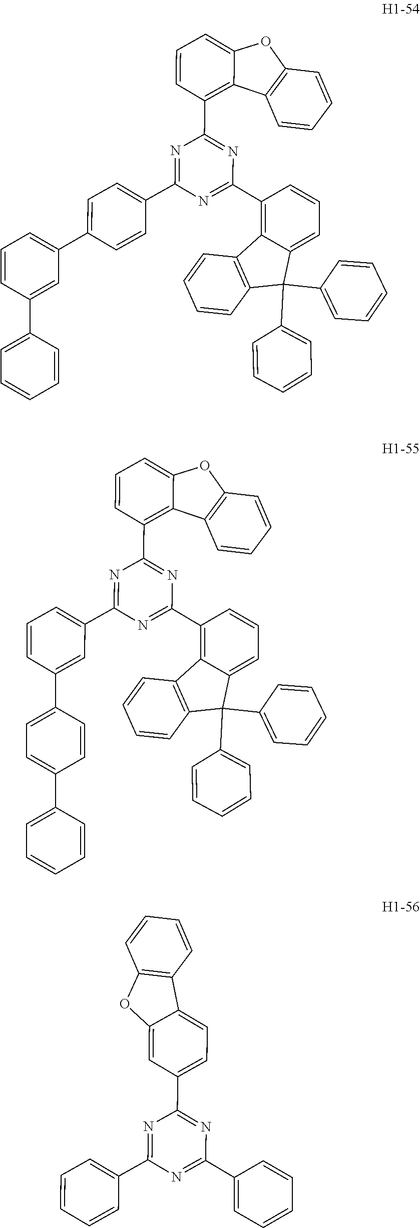 [SMILES: c1ccc(-c2cccc(-c3ccc(-c4nc(-c5cccc6c5-c5ccccc5C6(c5ccccc5)c5ccccc5)nc(-c5cccc6oc7ccccc7c56)n4)cc3)c2)cc1 ; c1ccc(-c2ccc(-c3cccc(-c4nc(-c5cccc6c5-c5ccccc5C6(c5ccccc5)c5ccccc5)nc(-c5cccc6oc7ccccc7c56)n4)c3)cc2)cc1 ; c1ccc(-c2nc(-c3ccccc3)nc(-c3ccc4c(c3)oc3ccccc34)n2)cc1]

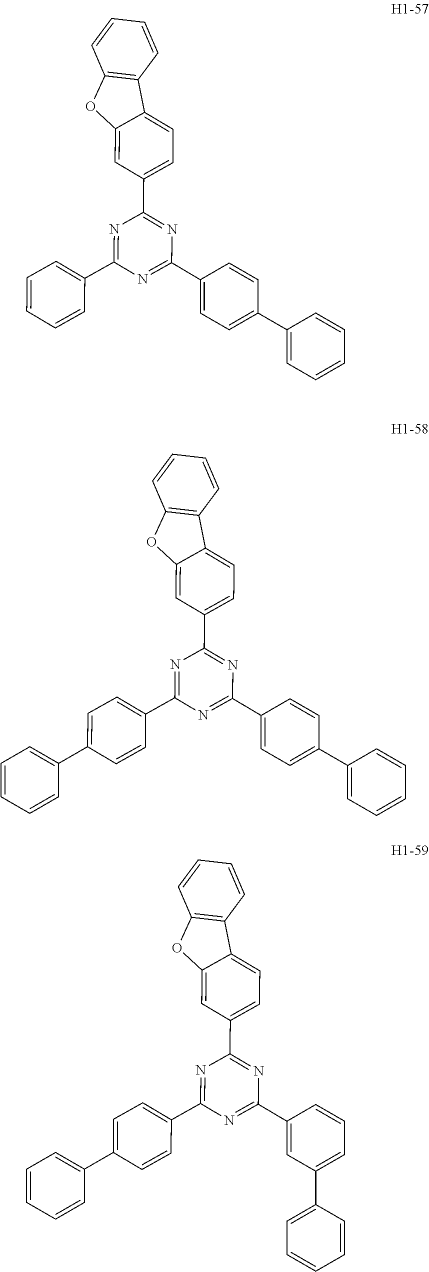 [SMILES: c1ccc(-c2ccc(-c3nc(-c4ccccc4)nc(-c4ccc5c(c4)oc4ccccc45)n3)cc2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4cccc(-c5ccccc5)c4)nc(-c4ccc5c(c4)oc4ccccc45)n3)cc2)cc1]

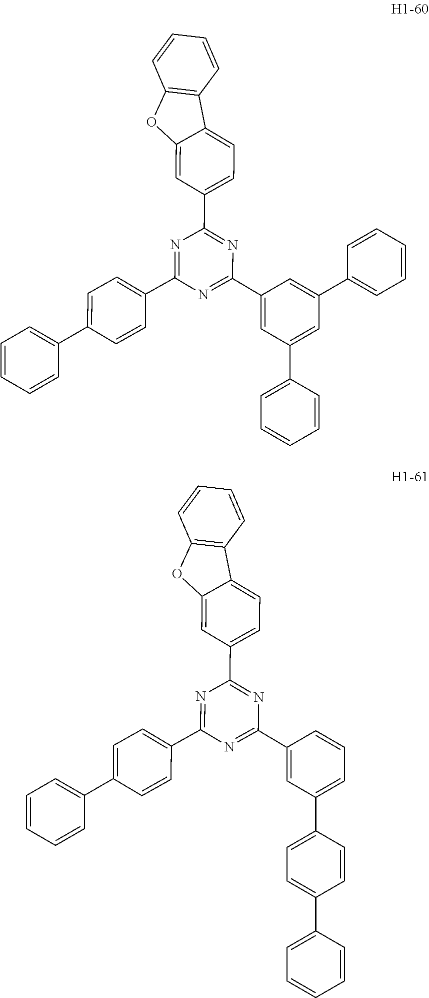 [SMILES: c1ccc(-c2ccc(-c3nc(-c4cc(-c5ccccc5)cc(-c5ccccc5)c4)nc(-c4ccc5c(c4)oc4ccccc45)n3)cc2)cc1 ; c1ccc(-c2ccc(-c3cccc(-c4nc(-c5ccc(-c6ccccc6)cc5)nc(-c5ccc6c(c5)oc5ccccc56)n4)c3)cc2)cc1]

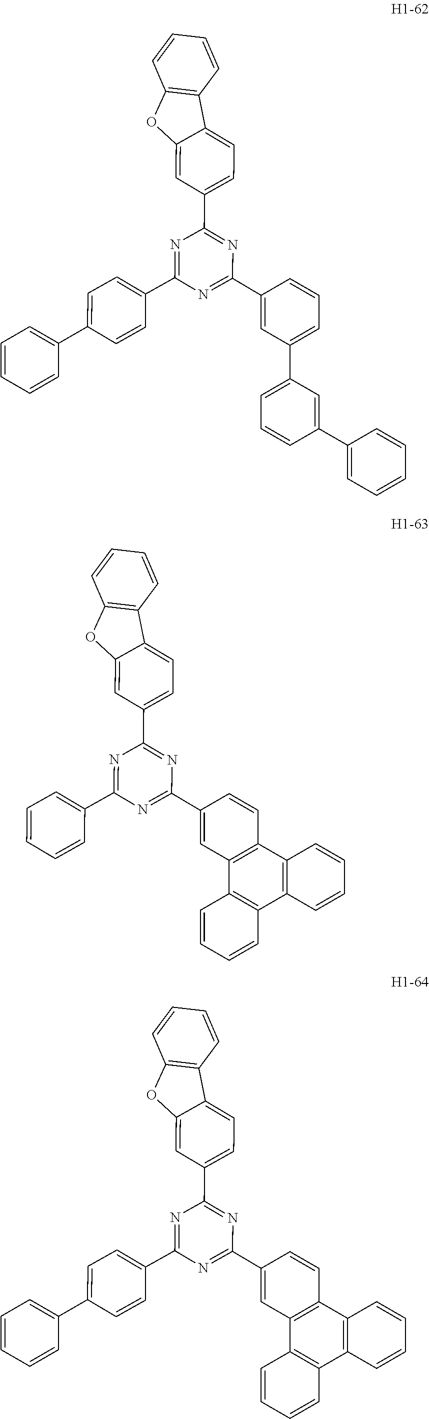 [SMILES: c1ccc(-c2ccc(-c3nc(-c4cccc(-c5cccc(-c6ccccc6)c5)c4)nc(-c4ccc5c(c4)oc4ccccc45)n3)cc2)cc1 ; c1ccc(-c2nc(-c3ccc4c(c3)oc3ccccc34)nc(-c3ccc4c5ccccc5c5ccccc5c4c3)n2)cc1 ; c1ccc(-c2ccc(-c3nc(-c4ccc5c(c4)oc4ccccc45)nc(-c4ccc5c6ccccc6c6ccccc6c5c4)n3)cc2)cc1]

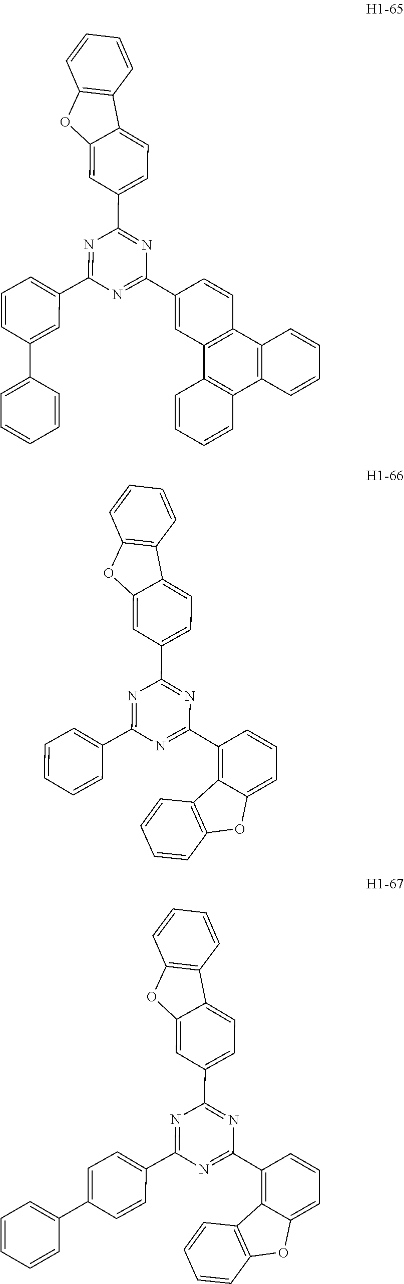 [SMILES: c1ccc(-c2cccc(-c3nc(-c4ccc5c(c4)oc4ccccc45)nc(-c4ccc5c6ccccc6c6ccccc6c5c4)n3)c2)cc1 ; c1ccc(-c2nc(-c3ccc4c(c3)oc3ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)cc2)cc1]

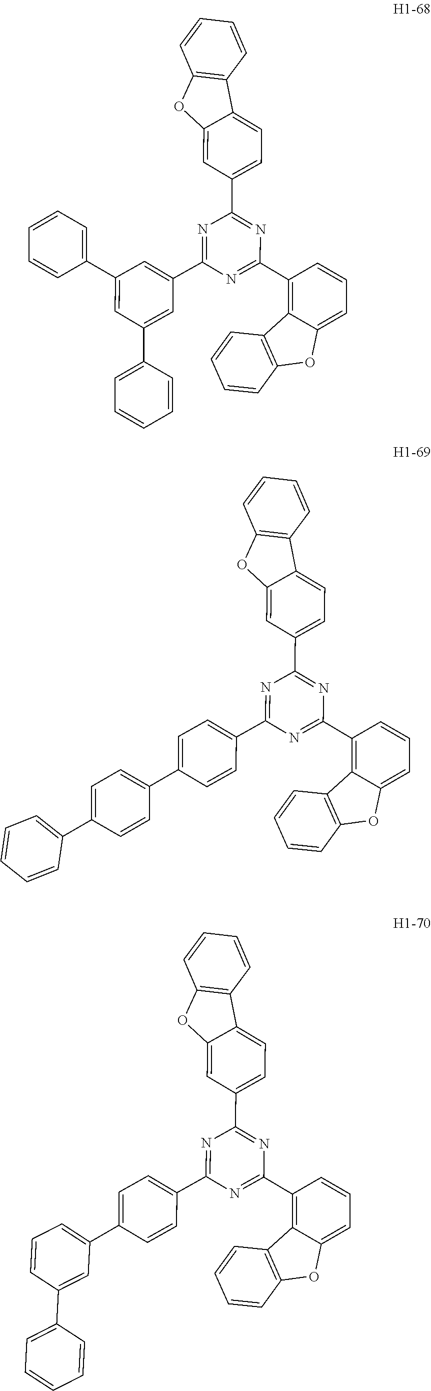 [SMILES: c1ccc(-c2cc(-c3ccccc3)cc(-c3nc(-c4ccc5c(c4)oc4ccccc45)nc(-c4cccc5oc6ccccc6c45)n3)c2)cc1 ; c1ccc(-c2ccc(-c3ccc(-c4nc(-c5ccc6c(c5)oc5ccccc56)nc(-c5cccc6oc7ccccc7c56)n4)cc3)cc2)cc1 ; c1ccc(-c2cccc(-c3ccc(-c4nc(-c5ccc6c(c5)oc5ccccc56)nc(-c5cccc6oc7ccccc7c56)n4)cc3)c2)cc1]

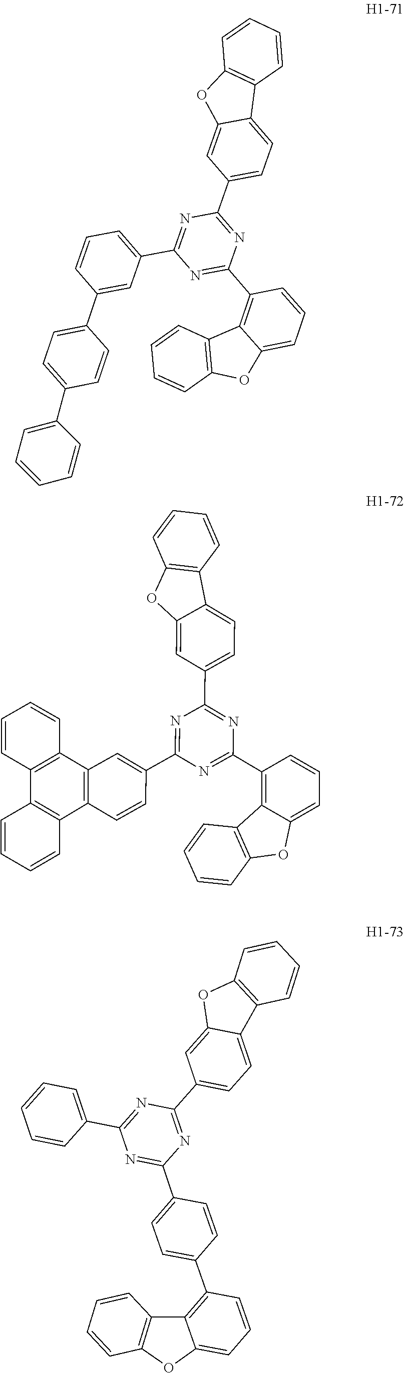 [SMILES: c1ccc(-c2ccc(-c3cccc(-c4nc(-c5ccc6c(c5)oc5ccccc56)nc(-c5cccc6oc7ccccc7c56)n4)c3)cc2)cc1 ; c1ccc2c(c1)oc1cc(-c3nc(-c4ccc5c6ccccc6c6ccccc6c5c4)nc(-c4cccc5oc6ccccc6c45)n3)ccc12 ; c1ccc(-c2nc(-c3ccc(-c4cccc5oc6ccccc6c45)cc3)nc(-c3ccc4c(c3)oc3ccccc34)n2)cc1]

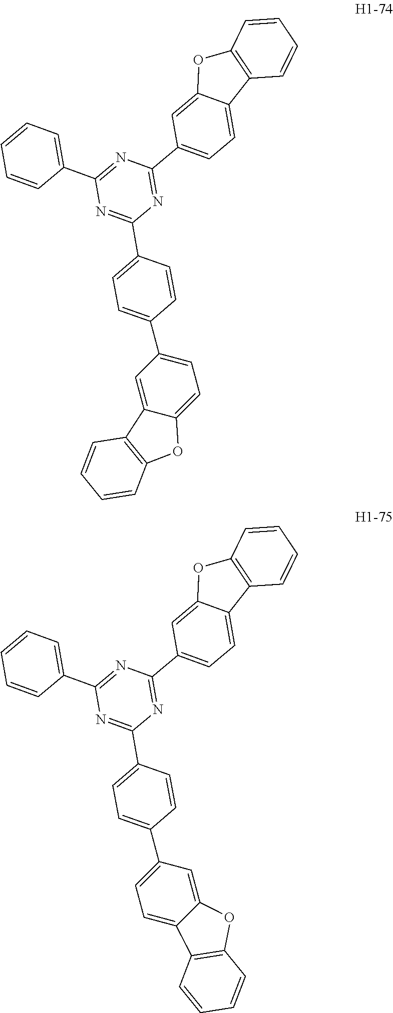 [SMILES: c1ccc(-c2nc(-c3ccc(-c4ccc5oc6ccccc6c5c4)cc3)nc(-c3ccc4c(c3)oc3ccccc34)n2)cc1 ; c1ccc(-c2nc(-c3ccc(-c4ccc5c(c4)oc4ccccc45)cc3)nc(-c3ccc4c(c3)oc3ccccc34)n2)cc1]

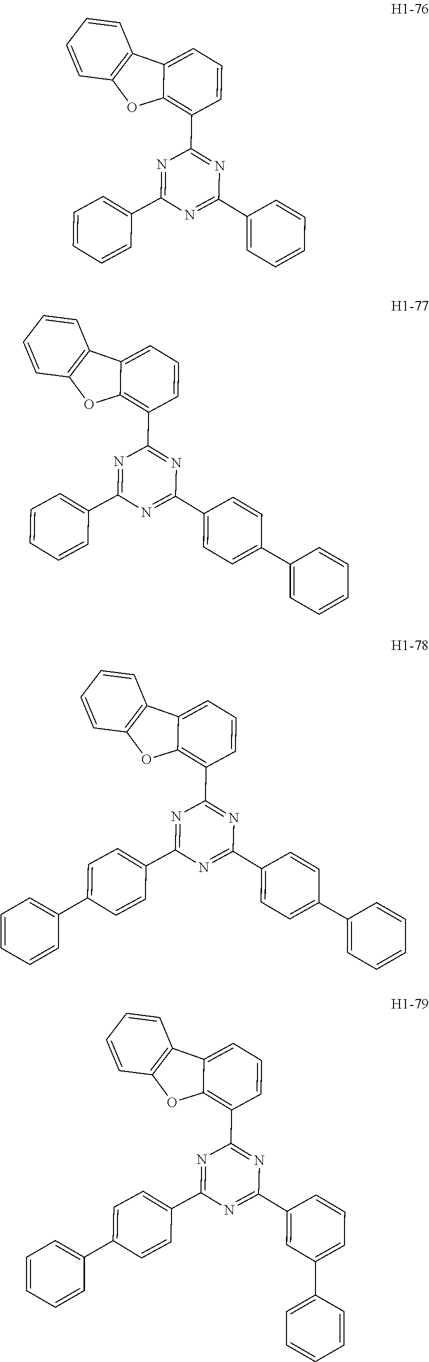 [SMILES: c1ccc(-c2nc(-c3ccccc3)nc(-c3cccc4c3oc3ccccc34)n2)cc1 ; c1ccc(-c2ccc(-c3nc(-c4ccccc4)nc(-c4cccc5c4oc4ccccc45)n3)cc2)cc1 ; c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4cccc(-c5ccccc5)c4)nc(-c4cccc5c4oc4ccccc45)n3)cc2)cc1]

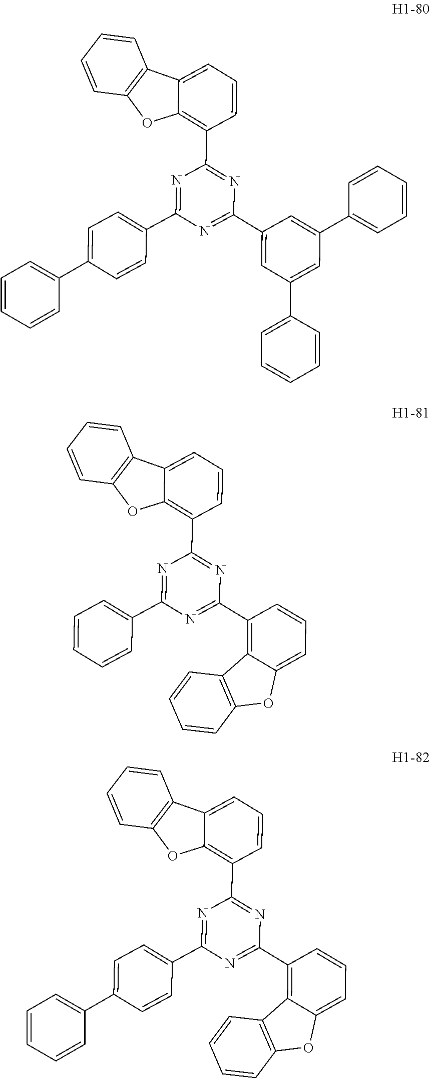 [SMILES: c1ccc(-c2ccc(-c3nc(-c4cc(-c5ccccc5)cc(-c5ccccc5)c4)nc(-c4cccc5c4oc4ccccc45)n3)cc2)cc1 ; c1ccc(-c2nc(-c3cccc4c3oc3ccccc34)nc(-c3cccc4oc5ccccc5c34)n2)cc1 ; c1ccc(-c2ccc(-c3nc(-c4cccc5c4oc4ccccc45)nc(-c4cccc5oc6ccccc6c45)n3)cc2)cc1]

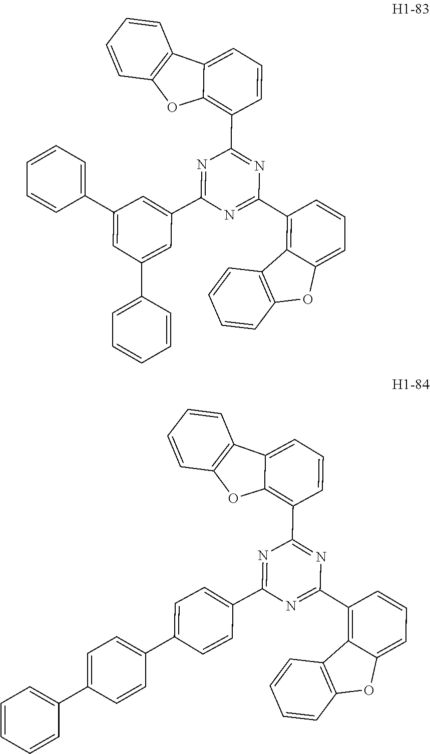 [SMILES: c1ccc(-c2cc(-c3ccccc3)cc(-c3nc(-c4cccc5c4oc4ccccc45)nc(-c4cccc5oc6ccccc6c45)n3)c2)cc1 ; c1ccc(-c2ccc(-c3ccc(-c4nc(-c5cccc6c5oc5ccccc56)nc(-c5cccc6oc7ccccc7c56)n4)cc3)cc2)cc1]

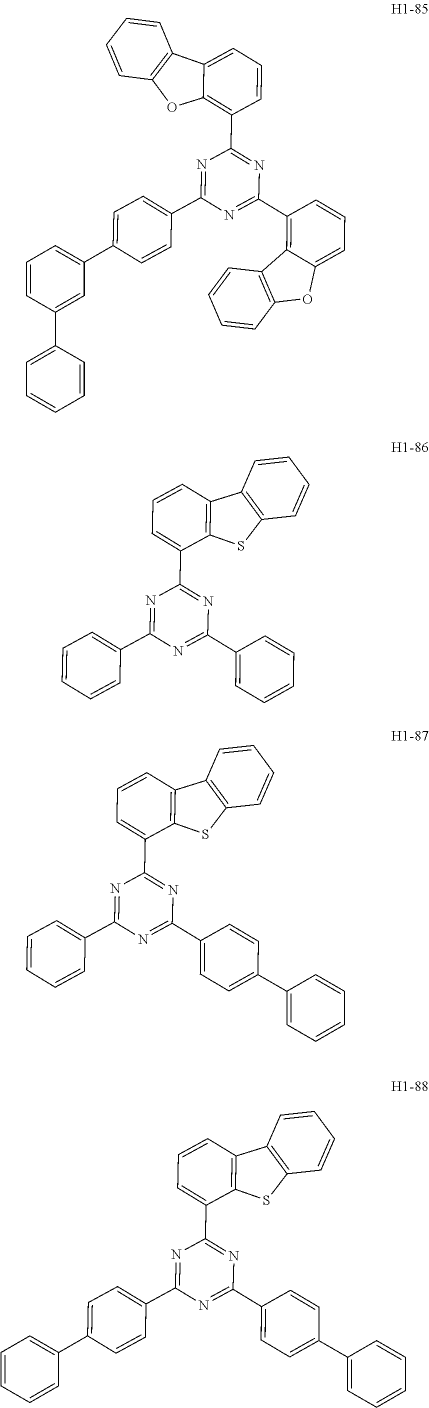

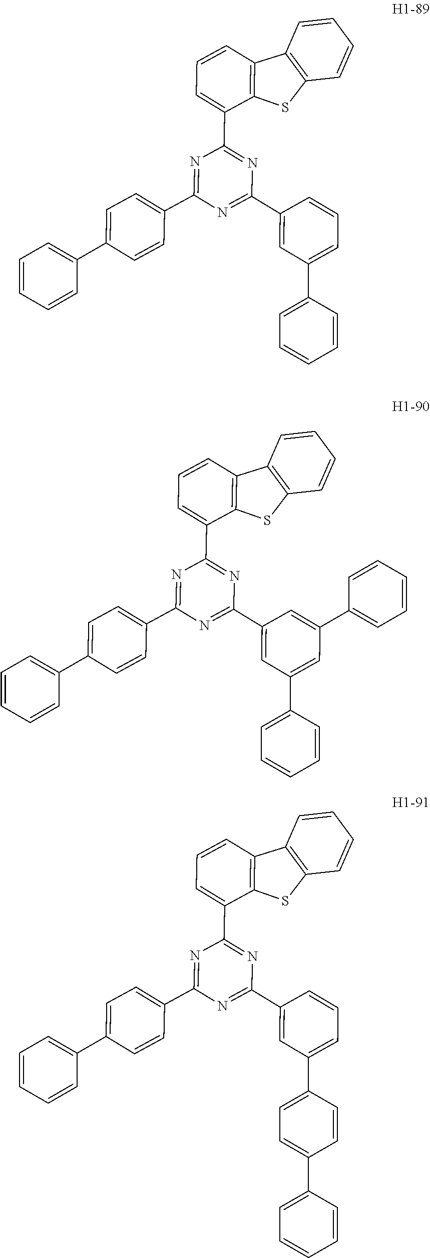

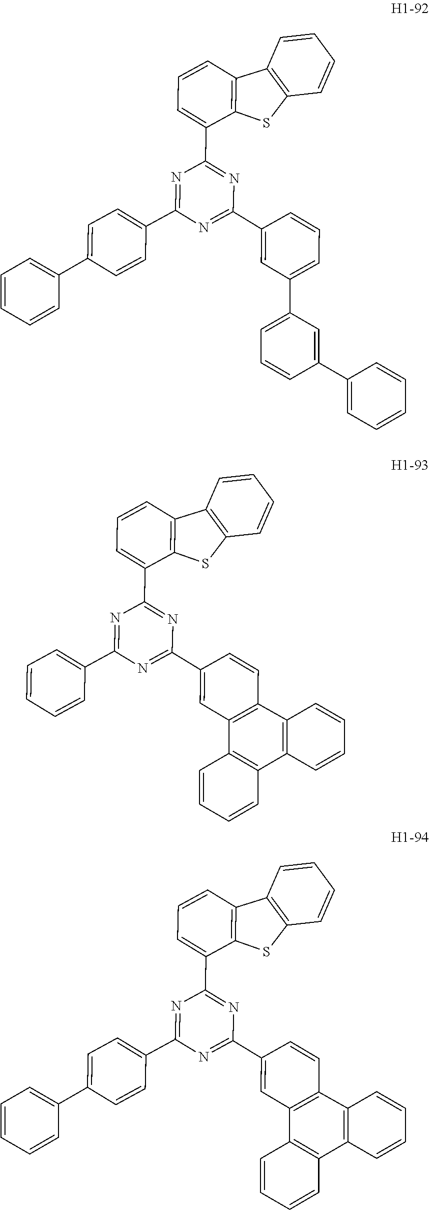 [SMILES: c1ccc(-c2ccc(-c3nc(-c4cccc(-c5cccc(-c6ccccc6)c5)c4)nc(-c4cccc5c4sc4ccccc45)n3)cc2)cc1 ; c1ccc(-c2nc(-c3ccc4c5ccccc5c5ccccc5c4c3)nc(-c3cccc4c3sc3ccccc34)n2)cc1 ; c1ccc(-c2ccc(-c3nc(-c4ccc5c6ccccc6c6ccccc6c5c4)nc(-c4cccc5c4sc4ccccc45)n3)cc2)cc1]

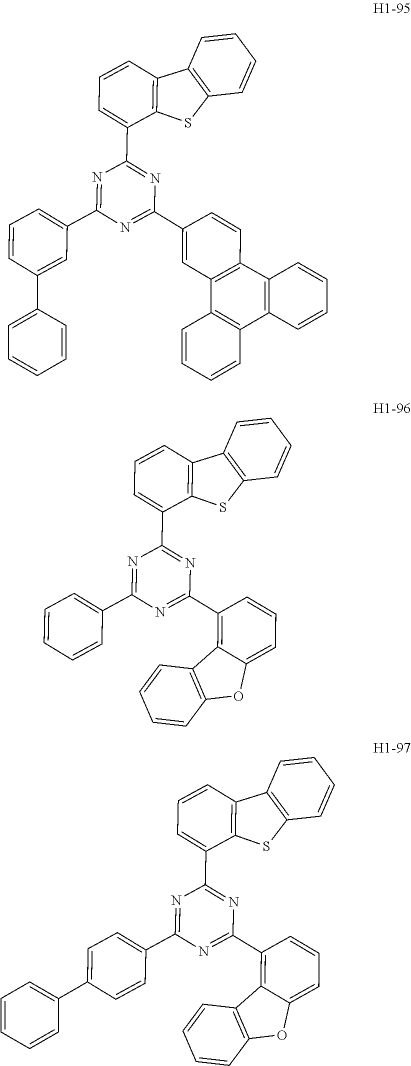 [SMILES: c1ccc(-c2cccc(-c3nc(-c4ccc5c6ccccc6c6ccccc6c5c4)nc(-c4cccc5c4sc4ccccc45)n3)c2)cc1 ; c1ccc(-c2nc(-c3cccc4c3sc3ccccc34)nc(-c3cccc4oc5ccccc5c34)n2)cc1 ; c1ccc(-c2ccc(-c3nc(-c4cccc5c4sc4ccccc45)nc(-c4cccc5oc6ccccc6c45)n3)cc2)cc1]

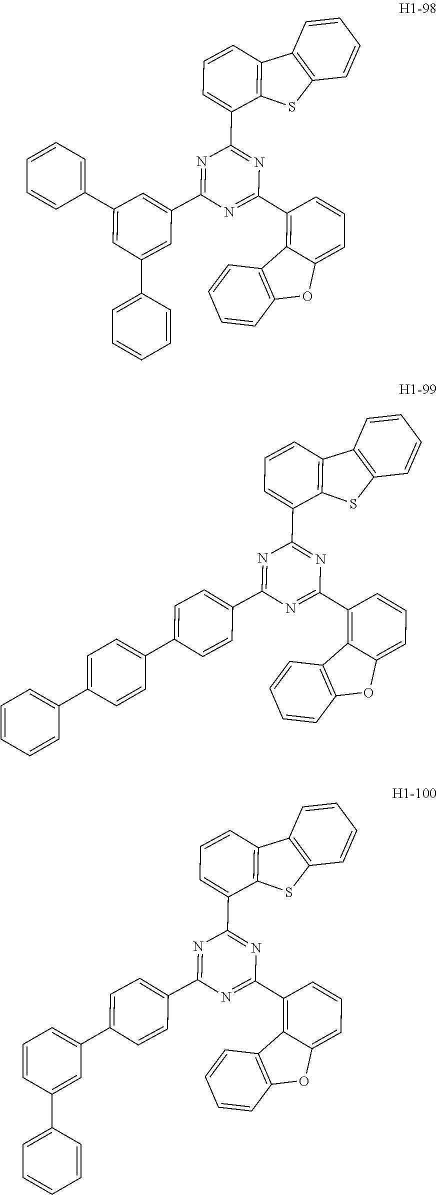 [SMILES: c1ccc(-c2cc(-c3ccccc3)cc(-c3nc(-c4cccc5c4sc4ccccc45)nc(-c4cccc5oc6ccccc6c45)n3)c2)cc1 ; c1ccc(-c2ccc(-c3ccc(-c4nc(-c5cccc6c5sc5ccccc56)nc(-c5cccc6oc7ccccc7c56)n4)cc3)cc2)cc1 ; c1ccc(-c2cccc(-c3ccc(-c4nc(-c5cccc6c5sc5ccccc56)nc(-c5cccc6oc7ccccc7c56)n4)cc3)c2)cc1]

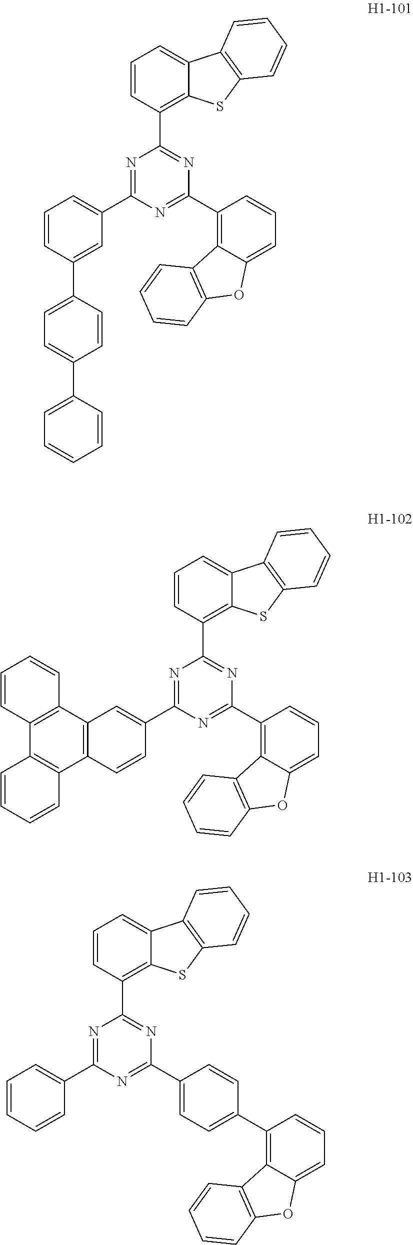 [SMILES: c1ccc(-c2ccc(-c3cccc(-c4nc(-c5cccc6c5sc5ccccc56)nc(-c5cccc6oc7ccccc7c56)n4)c3)cc2)cc1 ; c1ccc2c(c1)oc1cccc(-c3nc(-c4ccc5c6ccccc6c6ccccc6c5c4)nc(-c4cccc5c4sc4ccccc45)n3)c12 ; c1ccc(-c2nc(-c3ccc(-c4cccc5oc6ccccc6c45)cc3)nc(-c3cccc4c3sc3ccccc34)n2)cc1]

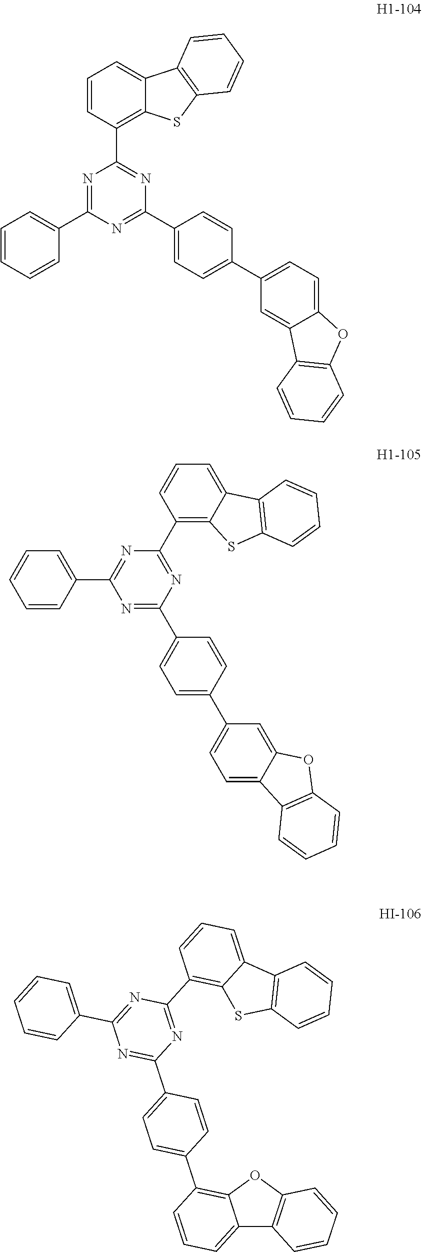 [SMILES: c1ccc(-c2nc(-c3ccc(-c4ccc5oc6ccccc6c5c4)cc3)nc(-c3cccc4c3sc3ccccc34)n2)cc1 ; c1ccc(-c2nc(-c3ccc(-c4ccc5c(c4)oc4ccccc45)cc3)nc(-c3cccc4c3sc3ccccc34)n2)cc1 ; c1ccc(-c2nc(-c3ccc(-c4cccc5c4oc4ccccc45)cc3)nc(-c3cccc4c3sc3ccccc34)n2)cc1]

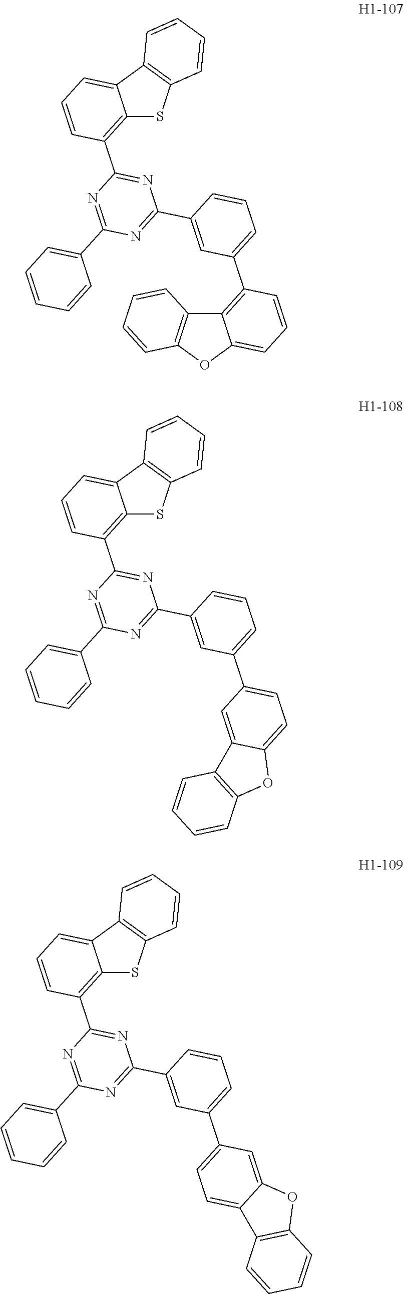 [SMILES: c1ccc(-c2nc(-c3cccc(-c4cccc5oc6ccccc6c45)c3)nc(-c3cccc4c3sc3ccccc34)n2)cc1 ; c1ccc(-c2nc(-c3cccc(-c4ccc5oc6ccccc6c5c4)c3)nc(-c3cccc4c3sc3ccccc34)n2)cc1 ; c1ccc(-c2nc(-c3cccc(-c4ccc5c(c4)oc4ccccc45)c3)nc(-c3cccc4c3sc3ccccc34)n2)cc1]

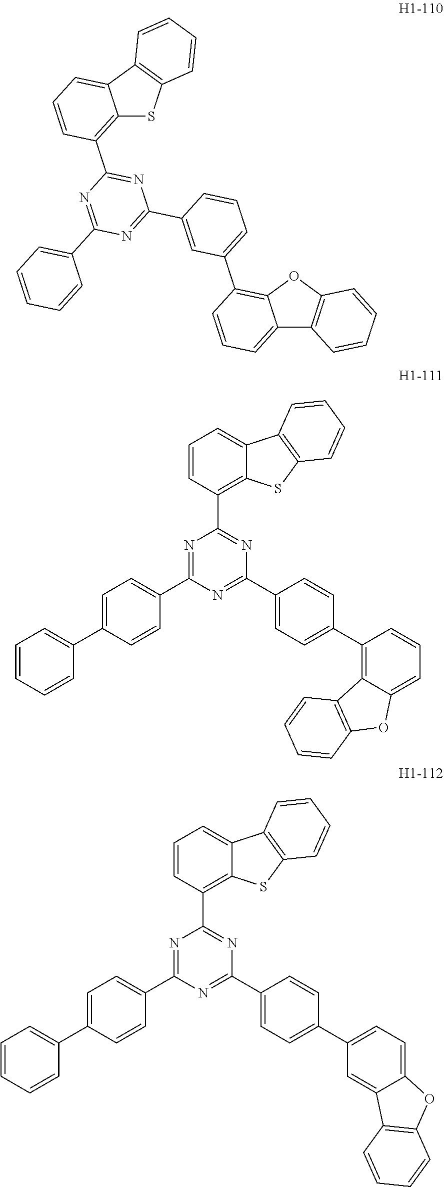

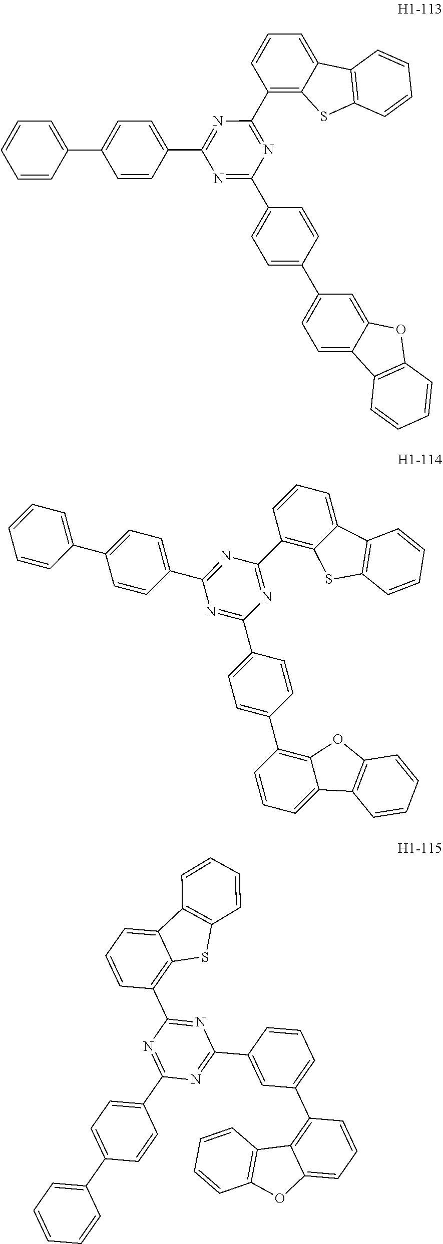 [SMILES: c1ccc(-c2ccc(-c3nc(-c4ccc(-c5ccc6c(c5)oc5ccccc56)cc4)nc(-c4cccc5c4sc4ccccc45)n3)cc2)cc1 ; c1ccc(-c2ccc(-c3nc(-c4ccc(-c5cccc6c5oc5ccccc56)cc4)nc(-c4cccc5c4sc4ccccc45)n3)cc2)cc1 ; c1ccc(-c2ccc(-c3nc(-c4cccc(-c5cccc6oc7ccccc7c56)c4)nc(-c4cccc5c4sc4ccccc45)n3)cc2)cc1]

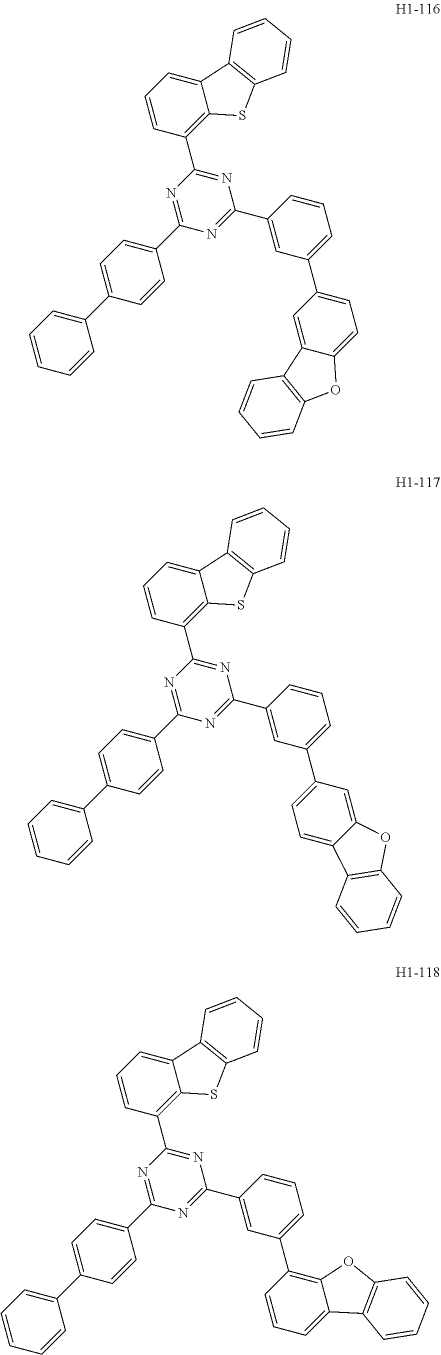 [SMILES: c1ccc(-c2ccc(-c3nc(-c4cccc(-c5ccc6oc7ccccc7c6c5)c4)nc(-c4cccc5c4sc4ccccc45)n3)cc2)cc1 ; c1ccc(-c2ccc(-c3nc(-c4cccc(-c5ccc6c(c5)oc5ccccc56)c4)nc(-c4cccc5c4sc4ccccc45)n3)cc2)cc1 ; c1ccc(-c2ccc(-c3nc(-c4cccc(-c5cccc6c5oc5ccccc56)c4)nc(-c4cccc5c4sc4ccccc45)n3)cc2)cc1]

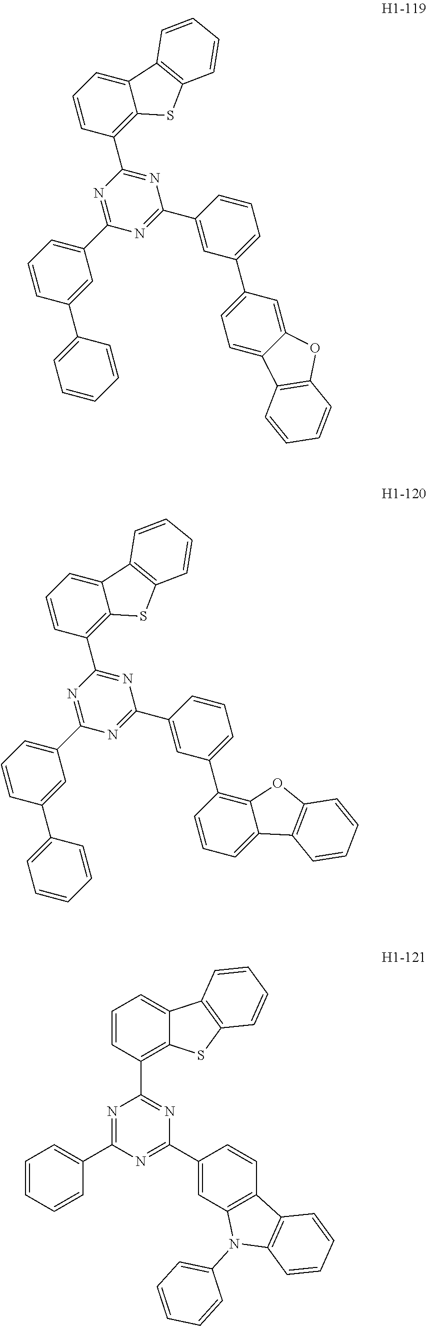 [SMILES: c1ccc(-c2cccc(-c3nc(-c4cccc(-c5ccc6c(c5)oc5ccccc56)c4)nc(-c4cccc5c4sc4ccccc45)n3)c2)cc1 ; c1ccc(-c2cccc(-c3nc(-c4cccc(-c5cccc6c5oc5ccccc56)c4)nc(-c4cccc5c4sc4ccccc45)n3)c2)cc1 ; c1ccc(-c2nc(-c3ccc4c5ccccc5n(-c5ccccc5)c4c3)nc(-c3cccc4c3sc3ccccc34)n2)cc1]

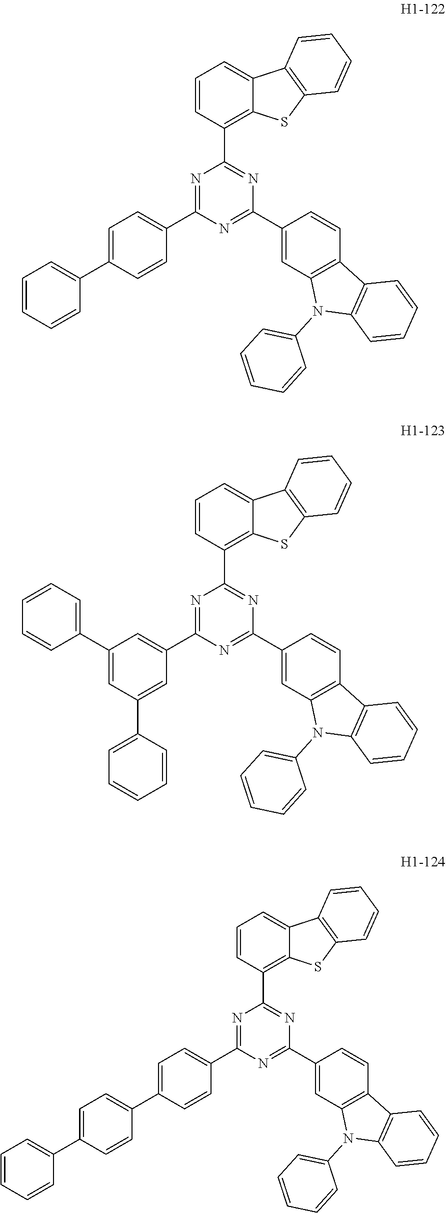 [SMILES: c1ccc(-c2ccc(-c3nc(-c4ccc5c6ccccc6n(-c6ccccc6)c5c4)nc(-c4cccc5c4sc4ccccc45)n3)cc2)cc1 ; c1ccc(-c2cc(-c3ccccc3)cc(-c3nc(-c4ccc5c6ccccc6n(-c6ccccc6)c5c4)nc(-c4cccc5c4sc4ccccc45)n3)c2)cc1 ; c1ccc(-c2ccc(-c3ccc(-c4nc(-c5ccc6c7ccccc7n(-c7ccccc7)c6c5)nc(-c5cccc6c5sc5ccccc56)n4)cc3)cc2)cc1]

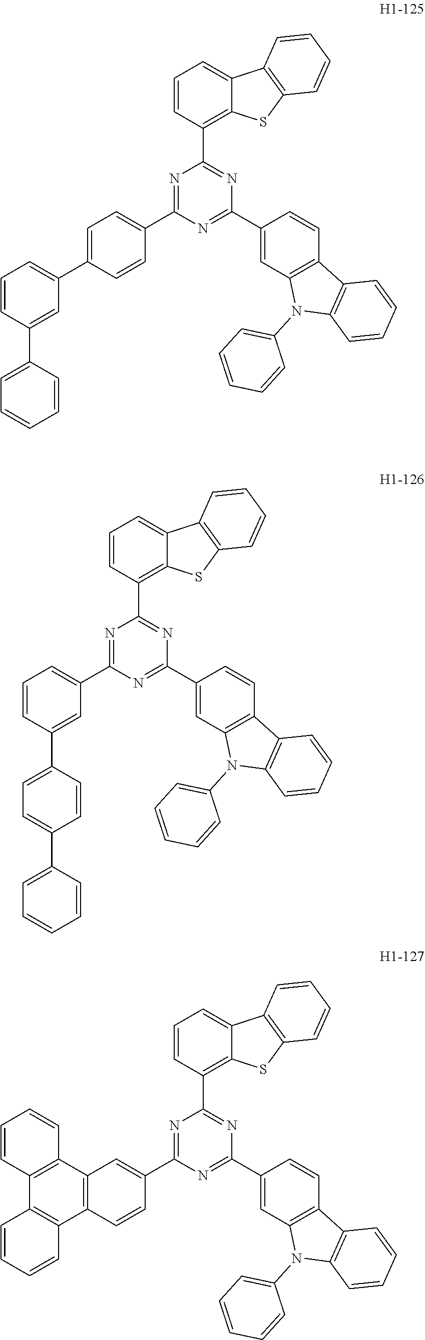 [SMILES: c1ccc(-c2cccc(-c3ccc(-c4nc(-c5ccc6c7ccccc7n(-c7ccccc7)c6c5)nc(-c5cccc6c5sc5ccccc56)n4)cc3)c2)cc1 ; c1ccc(-c2ccc(-c3cccc(-c4nc(-c5ccc6c7ccccc7n(-c7ccccc7)c6c5)nc(-c5cccc6c5sc5ccccc56)n4)c3)cc2)cc1 ; c1ccc(-n2c3ccccc3c3ccc(-c4nc(-c5ccc6c7ccccc7c7ccccc7c6c5)nc(-c5cccc6c5sc5ccccc56)n4)cc32)cc1]

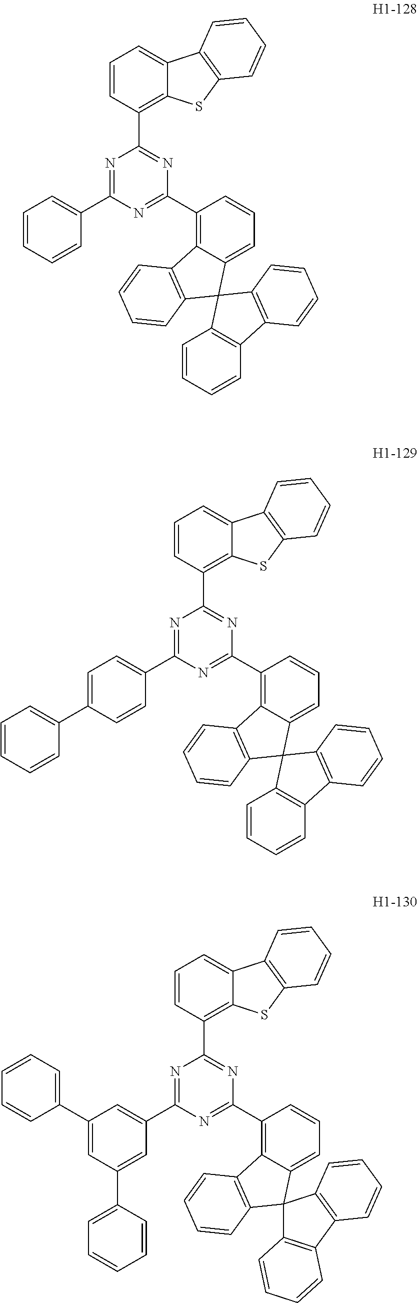 [SMILES: c1ccc(-c2nc(-c3cccc4c3-c3ccccc3C43c4ccccc4-c4ccccc43)nc(-c3cccc4c3sc3ccccc34)n2)cc1 ; c1ccc(-c2ccc(-c3nc(-c4cccc5c4-c4ccccc4C54c5ccccc5-c5ccccc54)nc(-c4cccc5c4sc4ccccc45)n3)cc2)cc1 ; c1ccc(-c2cc(-c3ccccc3)cc(-c3nc(-c4cccc5c4-c4ccccc4C54c5ccccc5-c5ccccc54)nc(-c4cccc5c4sc4ccccc45)n3)c2)cc1]

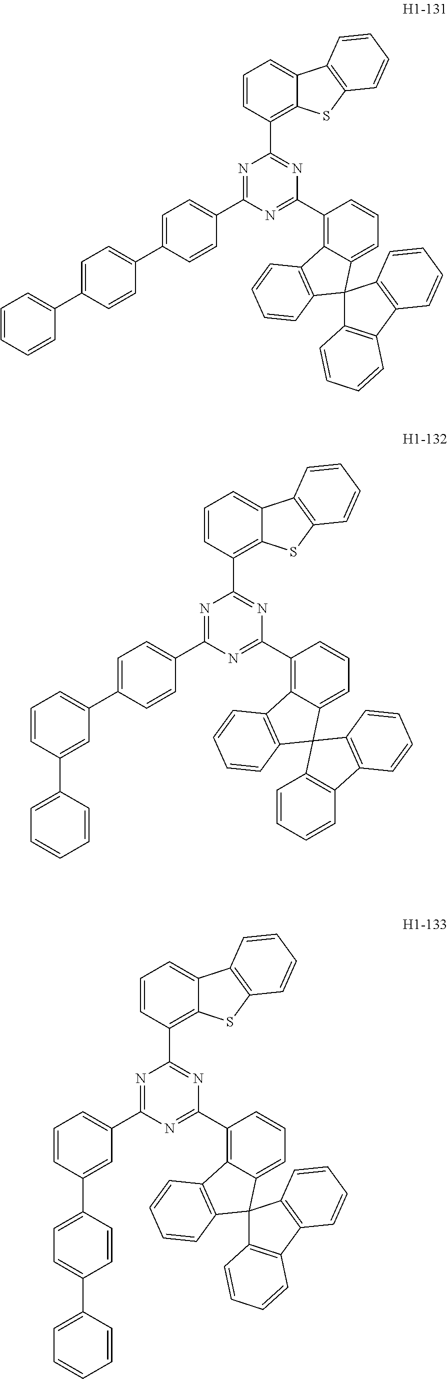 [SMILES: c1ccc(-c2ccc(-c3ccc(-c4nc(-c5cccc6c5-c5ccccc5C65c6ccccc6-c6ccccc65)nc(-c5cccc6c5sc5ccccc56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6c5sc5ccccc56)n4)cc3)c2)cc1 ; c1ccc(-c2ccc(-c3cccc(-c4nc(-c5cccc6c5-c5ccccc5C65c6ccccc6-c6ccccc65)nc(-c5cccc6c5sc5ccccc56)n4)c3)cc2)cc1]

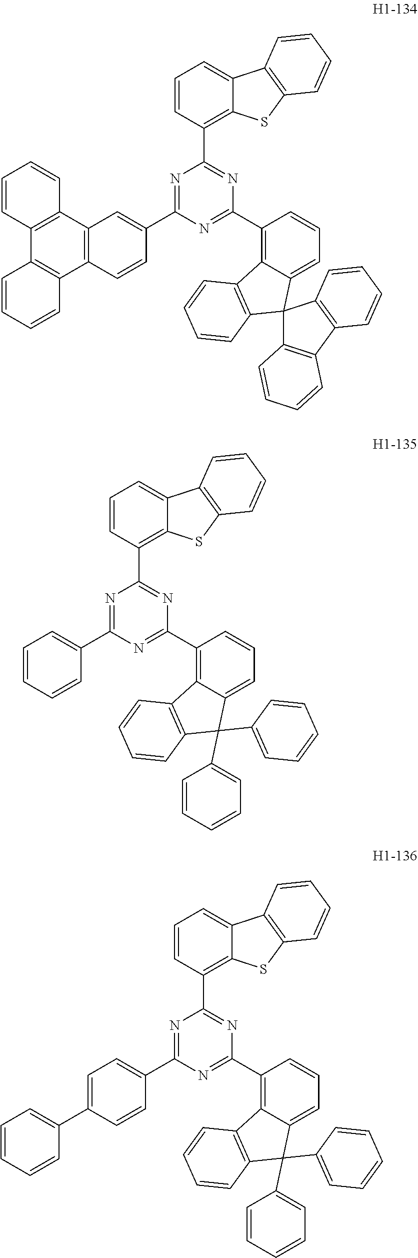 [SMILES: c1ccc2c(c1)-c1ccccc1C21c2ccccc2-c2c(-c3nc(-c4ccc5c6ccccc6c6ccccc6c5c4)nc(-c4cccc5c4sc4ccccc45)n3)cccc21 ; c1ccc(-c2nc(-c3cccc4c3-c3ccccc3C4(c3ccccc3)c3ccccc3)nc(-c3cccc4c3sc3ccccc34)n2)cc1 ; c1ccc(-c2ccc(-c3nc(-c4cccc5c4-c4ccccc4C5(c4ccccc4)c4ccccc4)nc(-c4cccc5c4sc4ccccc45)n3)cc2)cc1]

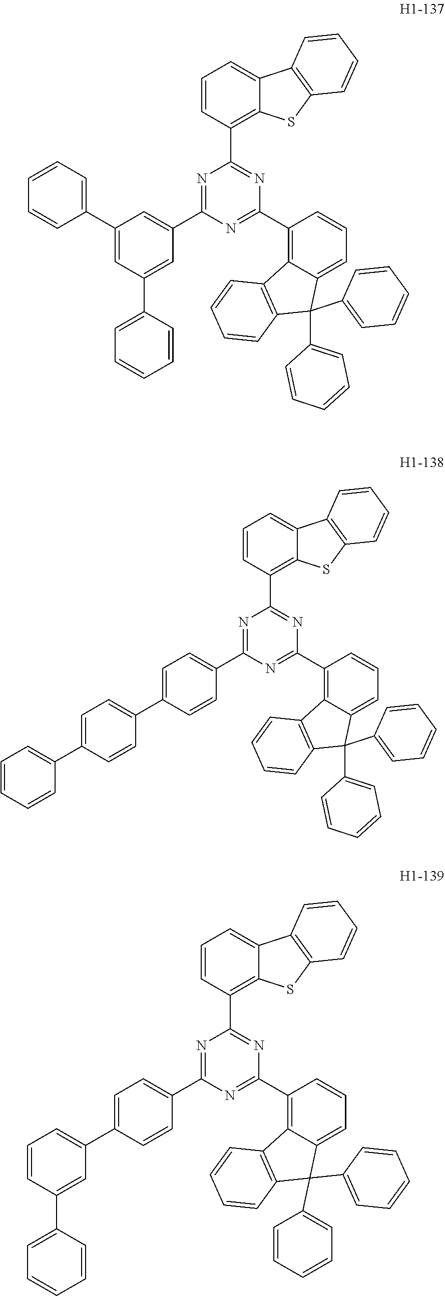 [SMILES: c1ccc(-c2cc(-c3ccccc3)cc(-c3nc(-c4cccc5c4-c4ccccc4C5(c4ccccc4)c4ccccc4)nc(-c4cccc5c4sc4ccccc45)n3)c2)cc1 ; c1ccc(-c2ccc(-c3ccc(-c4nc(-c5cccc6c5-c5ccccc5C6(c5ccccc5)c5ccccc5)nc(-c5cccc6c5sc5ccccc56)n4)cc3)cc2)cc1 ; c1ccc(-c2cccc(-c3ccc(-c4nc(-c5cccc6c5-c5ccccc5C6(c5ccccc5)c5ccccc5)nc(-c5cccc6c5sc5ccccc56)n4)cc3)c2)cc1]

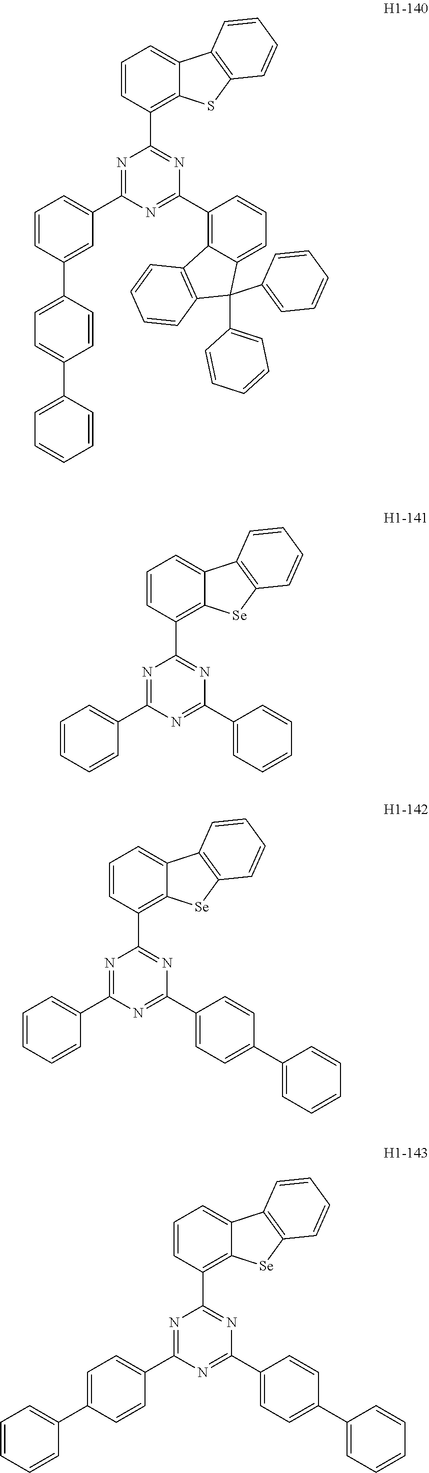 [SMILES: c1ccc(-c2ccc(-c3cccc(-c4nc(-c5cccc6c5-c5ccccc5C6(c5ccccc5)c5ccccc5)nc(-c5cccc6c5sc5ccccc56)n4)c3)cc2)cc1 ; c1ccc(-c2nc(-c3ccccc3)nc(-c3cccc4c3[se]c3ccccc34)n2)cc1 ; c1ccc(-c2ccc(-c3nc(-c4ccccc4)nc(-c4cccc5c4[se]c4ccccc45)n3)cc2)cc1 ; c1ccc(-c2ccc(-c3nc(-c4ccc(-c5ccccc5)cc4)nc(-c4cccc5c4[se]c4ccccc45)n3)cc2)cc1]

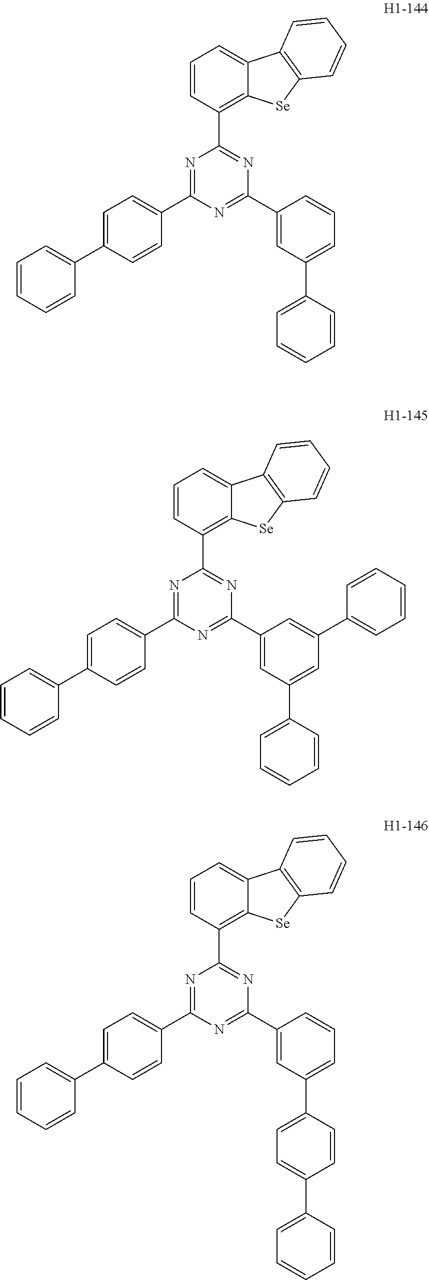 [SMILES: c1ccc(-c2ccc(-c3nc(-c4cccc(-c5ccccc5)c4)nc(-c4cccc5c4[se]c4ccccc45)n3)cc2)cc1 ; c1ccc(-c2ccc(-c3nc(-c4cc(-c5ccccc5)cc(-c5ccccc5)c4)nc(-c4cccc5c4[se]c4ccccc45)n3)cc2)cc1 ; c1ccc(-c2ccc(-c3cccc(-c4nc(-c5ccc(-c6ccccc6)cc5)nc(-c5cccc6c5[se]c5ccccc56)n4)c3)cc2)cc1]

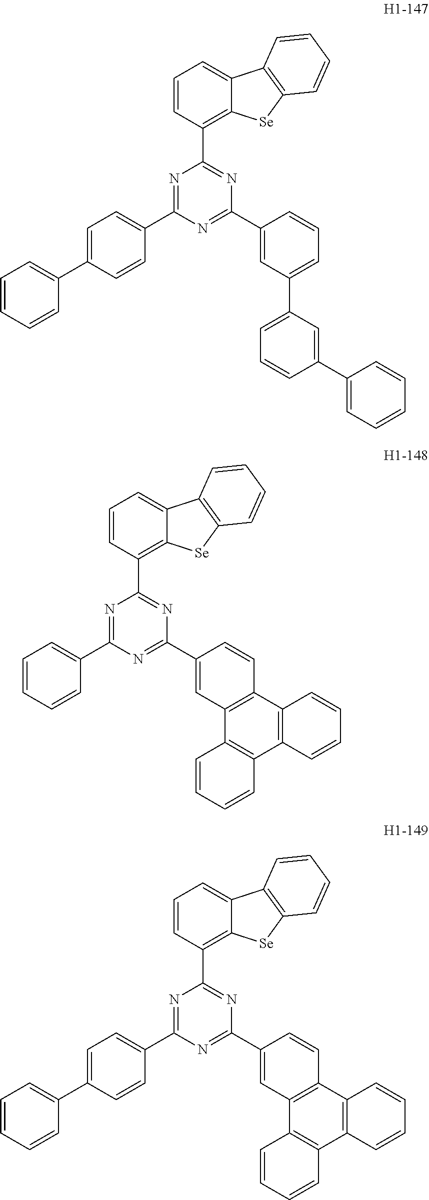 [SMILES: c1ccc(-c2ccc(-c3nc(-c4cccc(-c5cccc(-c6ccccc6)c5)c4)nc(-c4cccc5c4[se]c4ccccc45)n3)cc2)cc1 ; c1ccc(-c2nc(-c3ccc4c5ccccc5c5ccccc5c4c3)nc(-c3cccc4c3[se]c3ccccc34)n2)cc1 ; c1ccc(-c2ccc(-c3nc(-c4ccc5c6ccccc6c6ccccc6c5c4)nc(-c4cccc5c4[se]c4ccccc45)n3)cc2)cc1]

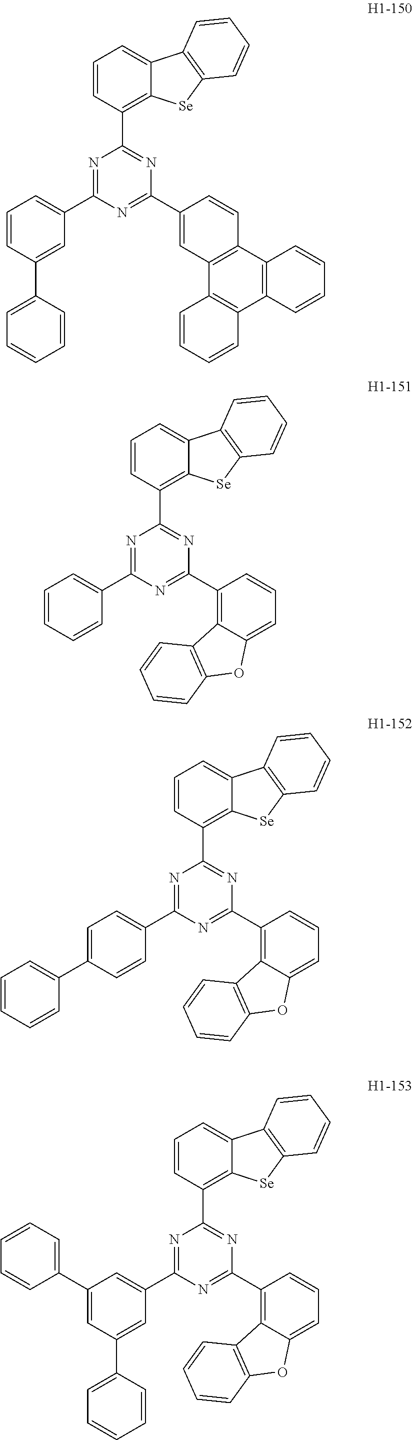

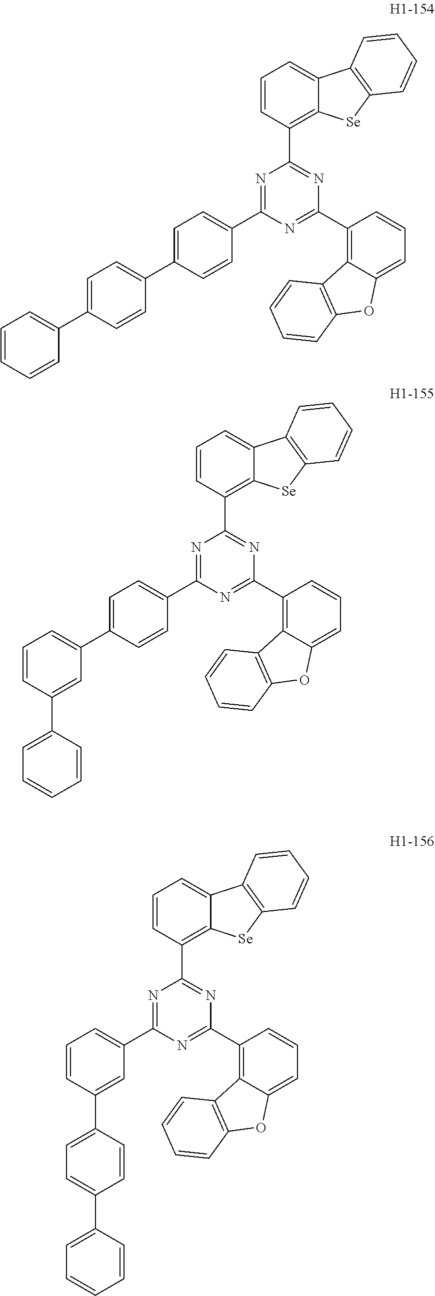 [SMILES: c1ccc(-c2ccc(-c3ccc(-c4nc(-c5cccc6c5[se]c5ccccc56)nc(-c5cccc6oc7ccccc7c56)n4)cc3)cc2)cc1 ; c1ccc(-c2cccc(-c3ccc(-c4nc(-c5cccc6c5[se]c5ccccc56)nc(-c5cccc6oc7ccccc7c56)n4)cc3)c2)cc1 ; c1ccc(-c2ccc(-c3cccc(-c4nc(-c5cccc6c5[se]c5ccccc56)nc(-c5cccc6oc7ccccc7c56)n4)c3)cc2)cc1]

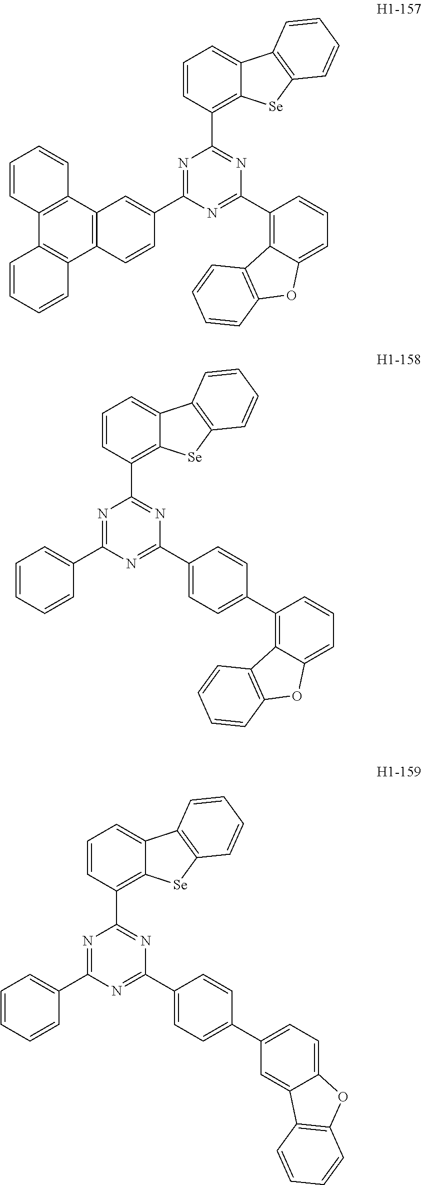

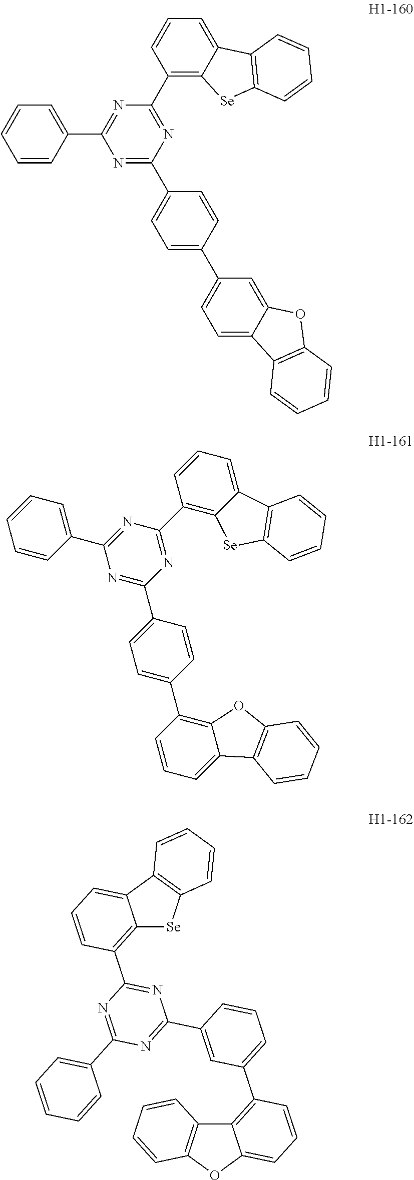 [SMILES: c1ccc(-c2nc(-c3ccc(-c4ccc5c(c4)oc4ccccc45)cc3)nc(-c3cccc4c3[se]c3ccccc34)n2)cc1 ; c1ccc(-c2nc(-c3ccc(-c4cccc5c4oc4ccccc45)cc3)nc(-c3cccc4c3[se]c3ccccc34)n2)cc1 ; c1ccc(-c2nc(-c3cccc(-c4cccc5oc6ccccc6c45)c3)nc(-c3cccc4c3[se]c3ccccc34)n2)cc1]

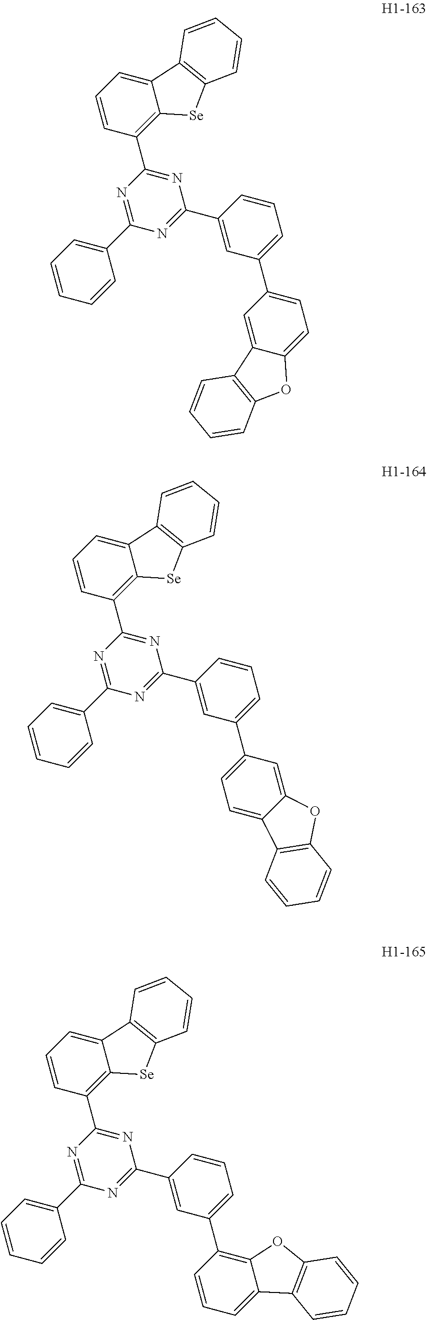 [SMILES: c1ccc(-c2nc(-c3cccc(-c4ccc5oc6ccccc6c5c4)c3)nc(-c3cccc4c3[se]c3ccccc34)n2)cc1 ; c1ccc(-c2nc(-c3cccc(-c4ccc5c(c4)oc4ccccc45)c3)nc(-c3cccc4c3[se]c3ccccc34)n2)cc1 ; c1ccc(-c2nc(-c3cccc(-c4cccc5c4oc4ccccc45)c3)nc(-c3cccc4c3[se]c3ccccc34)n2)cc1]

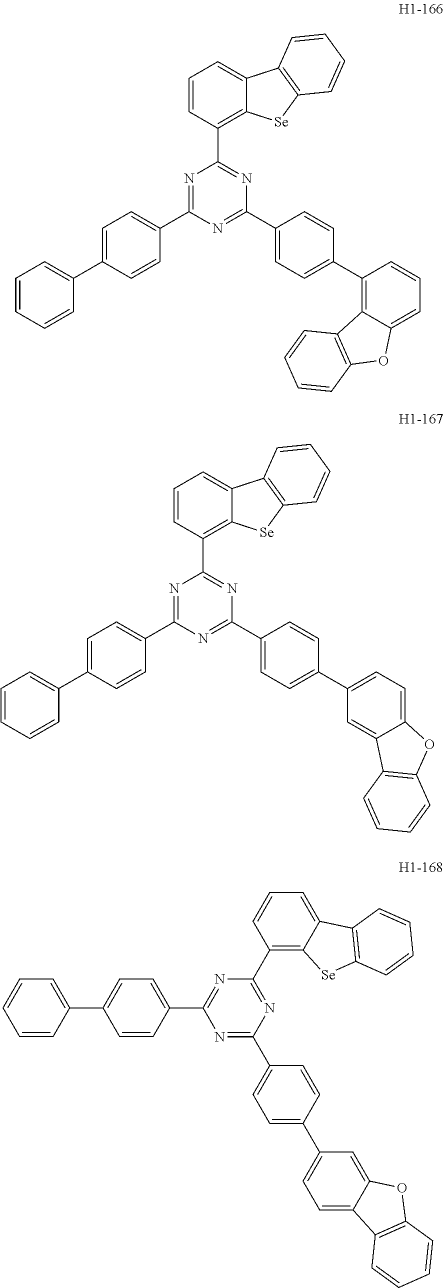 [SMILES: c1ccc(-c2ccc(-c3nc(-c4ccc(-c5cccc6oc7ccccc7c56)cc4)nc(-c4cccc5c4[se]c4ccccc45)n3)cc2)cc1 ; c1ccc(-c2ccc(-c3nc(-c4ccc(-c5ccc6oc7ccccc7c6c5)cc4)nc(-c4cccc5c4[se]c4ccccc45)n3)cc2)cc1 ; c1ccc(-c2ccc(-c3nc(-c4ccc(-c5ccc6c(c5)oc5ccccc56)cc4)nc(-c4cccc5c4[se]c4ccccc45)n3)cc2)cc1]

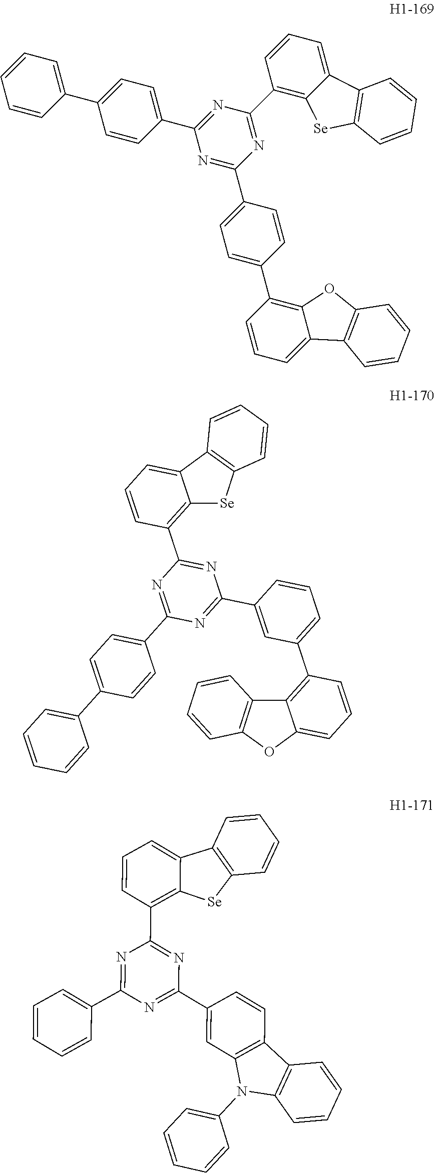 [SMILES: c1ccc(-c2ccc(-c3nc(-c4ccc(-c5cccc6c5oc5ccccc56)cc4)nc(-c4cccc5c4[se]c4ccccc45)n3)cc2)cc1 ; c1ccc(-c2ccc(-c3nc(-c4cccc(-c5cccc6oc7ccccc7c56)c4)nc(-c4cccc5c4[se]c4ccccc45)n3)cc2)cc1 ; c1ccc(-c2nc(-c3ccc4c5ccccc5n(-c5ccccc5)c4c3)nc(-c3cccc4c3[se]c3ccccc34)n2)cc1]

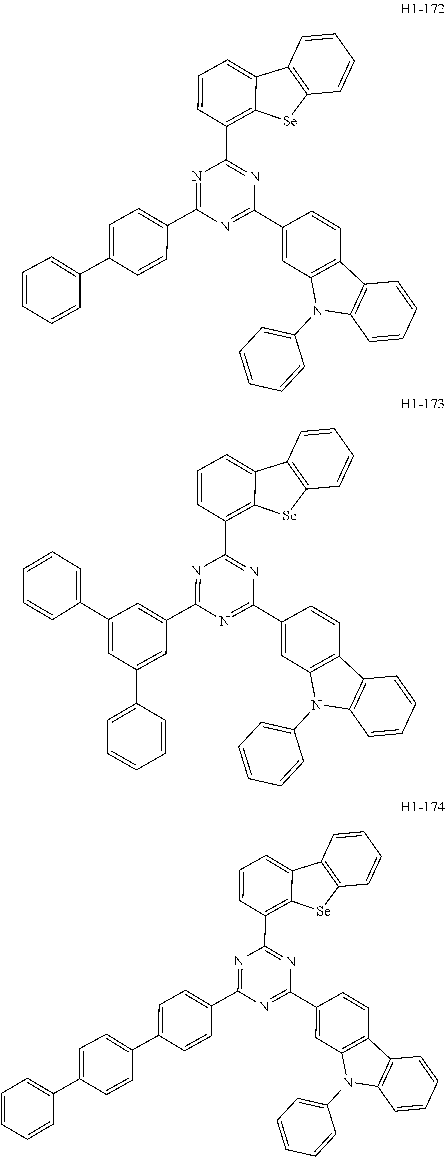

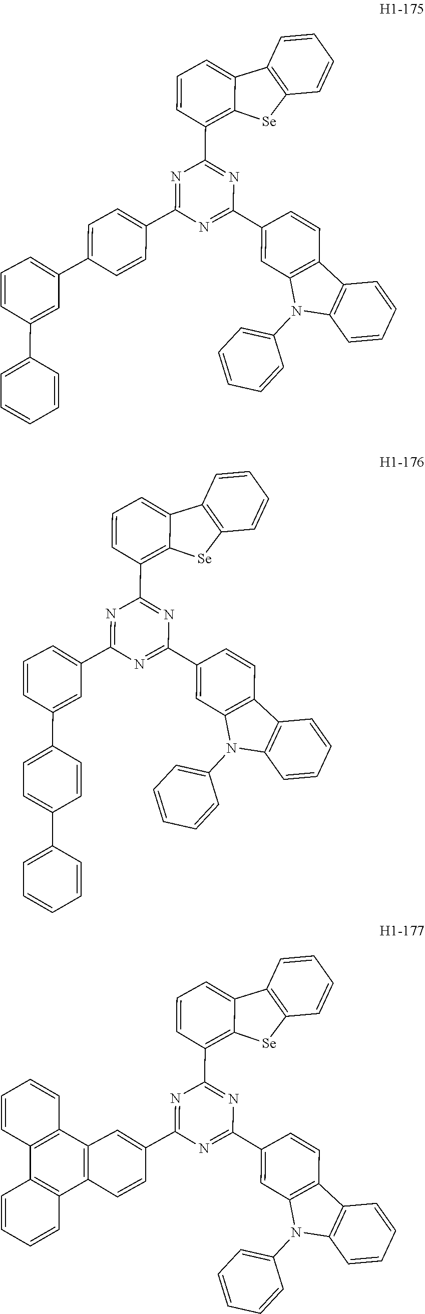 [SMILES: c1ccc(-c2cccc(-c3ccc(-c4nc(-c5ccc6c7ccccc7n(-c7ccccc7)c6c5)nc(-c5cccc6c5[se]c5ccccc56)n4)cc3)c2)cc1 ; c1ccc(-c2ccc(-c3cccc(-c4nc(-c5ccc6c7ccccc7n(-c7ccccc7)c6c5)nc(-c5cccc6c5[se]c5ccccc56)n4)c3)cc2)cc1 ; c1ccc(-n2c3ccccc3c3ccc(-c4nc(-c5ccc6c7ccccc7c7ccccc7c6c5)nc(-c5cccc6c5[se]c5ccccc56)n4)cc32)cc1]

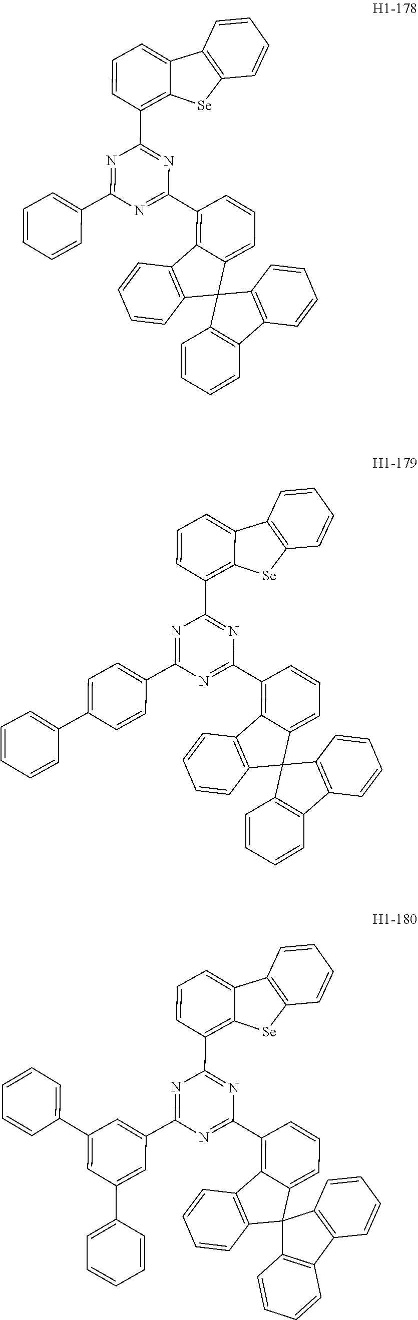 [SMILES: c1ccc(-c2nc(-c3cccc4c3-c3ccccc3C43c4ccccc4-c4ccccc43)nc(-c3cccc4c3[se]c3ccccc34)n2)cc1 ; c1ccc(-c2ccc(-c3nc(-c4cccc5c4-c4ccccc4C54c5ccccc5-c5ccccc54)nc(-c4cccc5c4[se]c4ccccc45)n3)cc2)cc1 ; c1ccc(-c2cc(-c3ccccc3)cc(-c3nc(-c4cccc5c4-c4ccccc4C54c5ccccc5-c5ccccc54)nc(-c4cccc5c4[se]c4ccccc45)n3)c2)cc1]

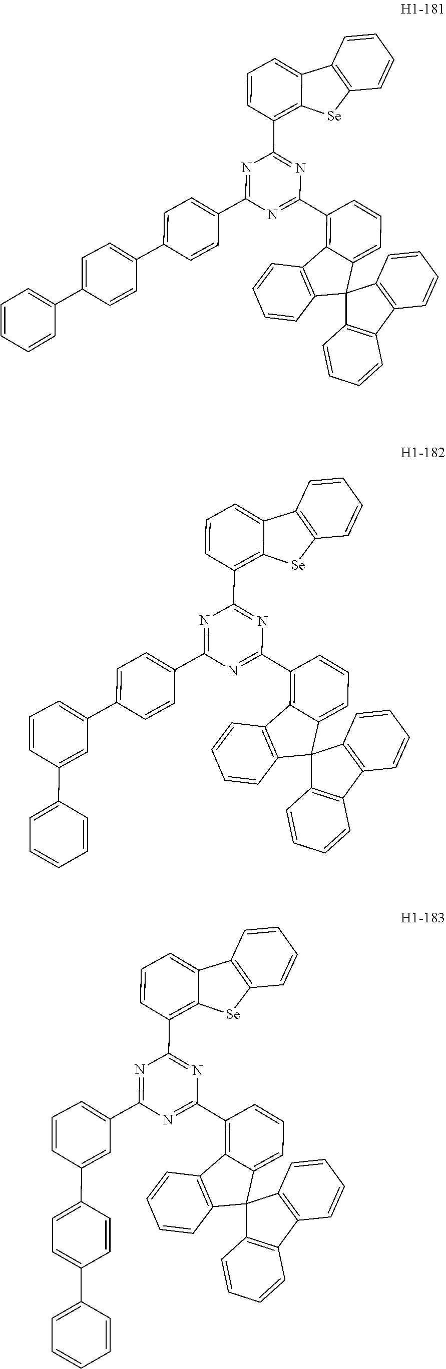 [SMILES: c1ccc(-c2ccc(-c3ccc(-c4nc(-c5cccc6c5-c5ccccc5C65c6ccccc6-c6ccccc65)nc(-c5cccc6c5[se]c5ccccc56)n4)cc3)cc2)cc1 ; c1ccc(-c2cccc(-c3ccc(-c4nc(-c5cccc6c5-c5ccccc5C65c6ccccc6-c6ccccc65)nc(-c5cccc6c5[se]c5ccccc56)n4)cc3)c2)cc1 ; c1ccc(-c2ccc(-c3cccc(-c4nc(-c5cccc6c5-c5ccccc5C65c6ccccc6-c6ccccc65)nc(-c5cccc6c5[se]c5ccccc56)n4)c3)cc2)cc1]

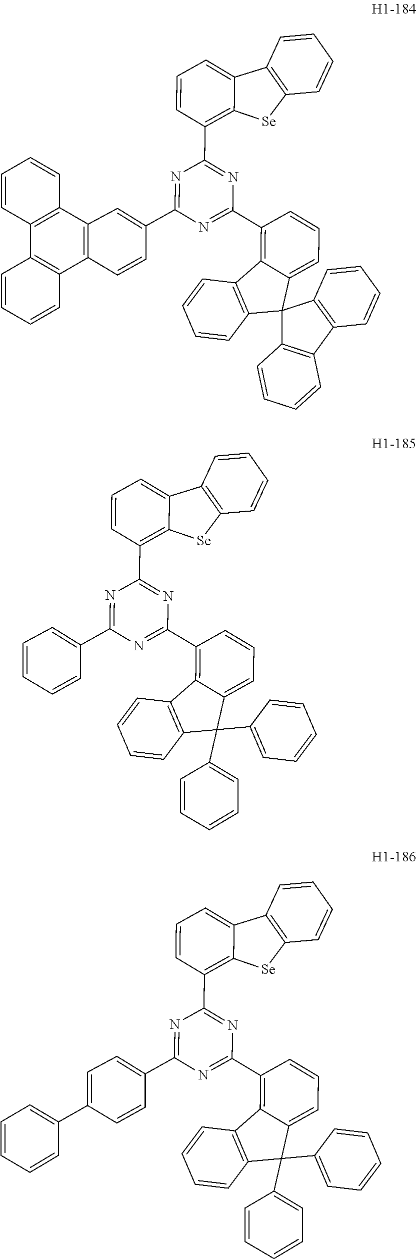 [SMILES: c1ccc2c(c1)-c1ccccc1C21c2ccccc2-c2c(-c3nc(-c4ccc5c6ccccc6c6ccccc6c5c4)nc(-c4cccc5c4[se]c4ccccc45)n3)cccc21 ; c1ccc(-c2nc(-c3cccc4c3-c3ccccc3C4(c3ccccc3)c3ccccc3)nc(-c3cccc4c3[se]c3ccccc34)n2)cc1 ; c1ccc(-c2ccc(-c3nc(-c4cccc5c4-c4ccccc4C5(c4ccccc4)c4ccccc4)nc(-c4cccc5c4[se]c4ccccc45)n3)cc2)cc1]

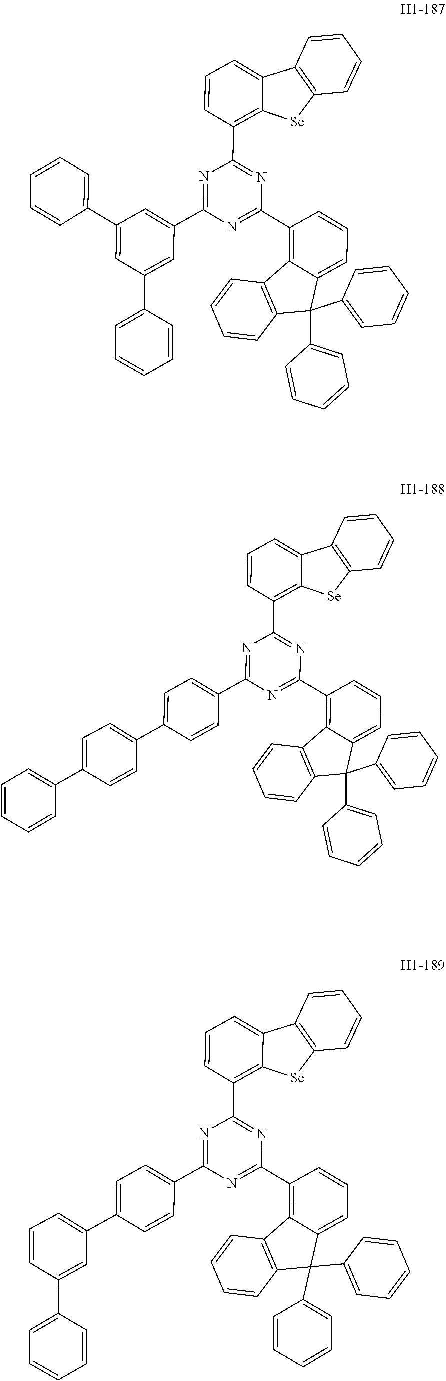 [SMILES: c1ccc(-c2cc(-c3ccccc3)cc(-c3nc(-c4cccc5c4-c4ccccc4C5(c4ccccc4)c4ccccc4)nc(-c4cccc5c4[se]c4ccccc45)n3)c2)cc1 ; c1ccc(-c2ccc(-c3ccc(-c4nc(-c5cccc6c5-c5ccccc5C6(c5ccccc5)c5ccccc5)nc(-c5cccc6c5[se]c5ccccc56)n4)cc3)cc2)cc1 ; c1ccc(-c2cccc(-c3ccc(-c4nc(-c5cccc6c5-c5ccccc5C6(c5ccccc5)c5ccccc5)nc(-c5cccc6c5[se]c5ccccc56)n4)cc3)c2)cc1]

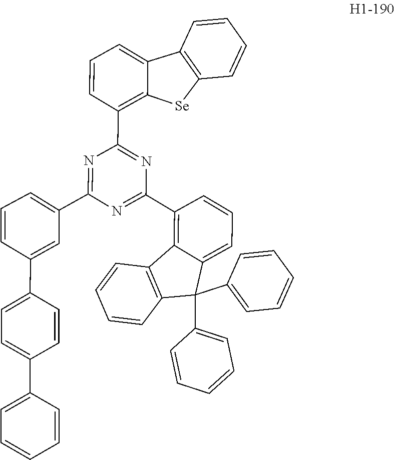 [SMILES: c1ccc(-c2ccc(-c3cccc(-c4nc(-c5cccc6c5-c5ccccc5C6(c5ccccc5)c5ccccc5)nc(-c5cccc6c5[se]c5ccccc56)n4)c3)cc2)cc1]

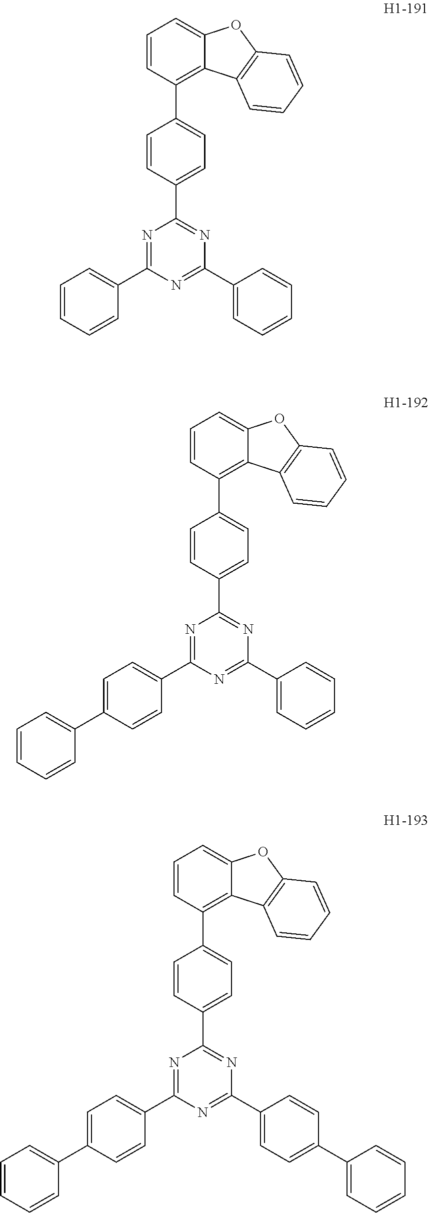 [SMILES: c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

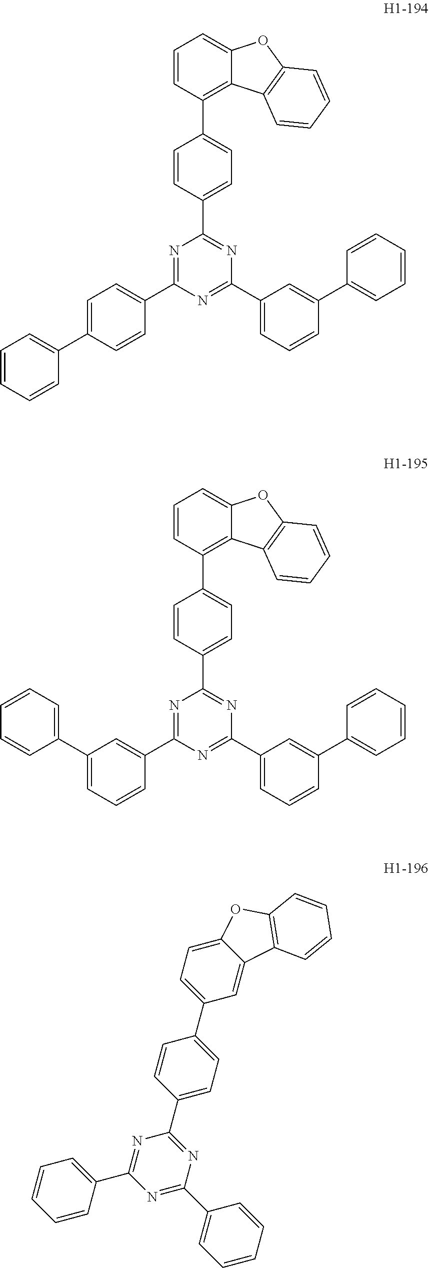 [SMILES: c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1 ; c1ccc(-c2nc(-c3ccccc3)nc(-c3ccc(-c4ccc5oc6ccccc6c5c4)cc3)n2)cc1]

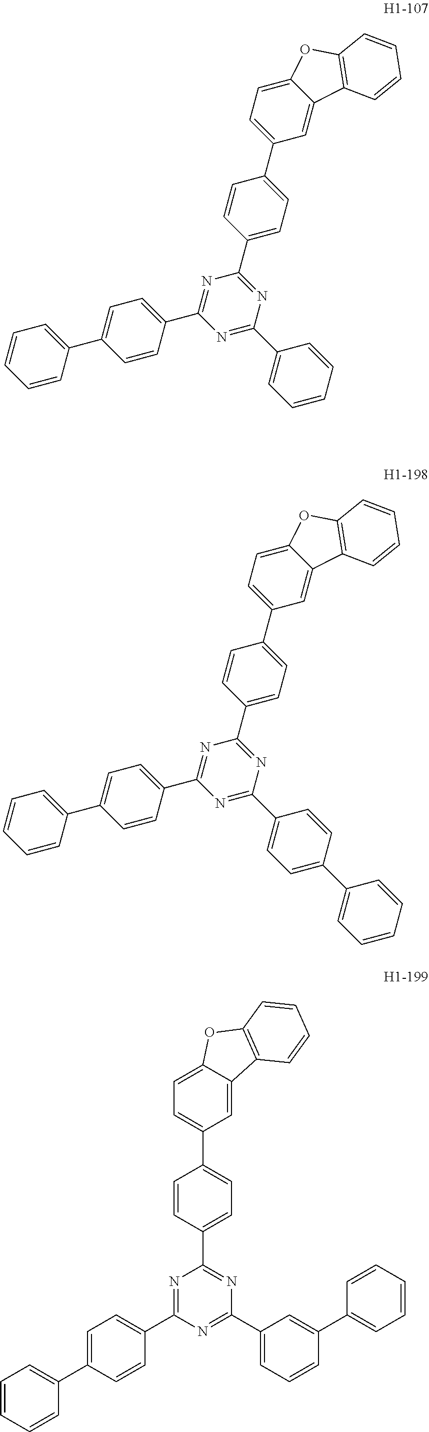 [SMILES: c1ccc(-c2ccc(-c3nc(-c4ccccc4)nc(-c4ccc(-c5ccc6oc7ccccc7c6c5)cc4)n3)cc2)cc1 ; c1ccc(-c2ccc(-c3nc(-c4ccc(-c5ccccc5)cc4)nc(-c4ccc(-c5ccc6oc7ccccc7c6c5)cc4)n3)cc2)cc1 ; c1ccc(-c2ccc(-c3nc(-c4ccc(-c5ccc6oc7ccccc7c6c5)cc4)nc(-c4cccc(-c5ccccc5)c4)n3)cc2)cc1]

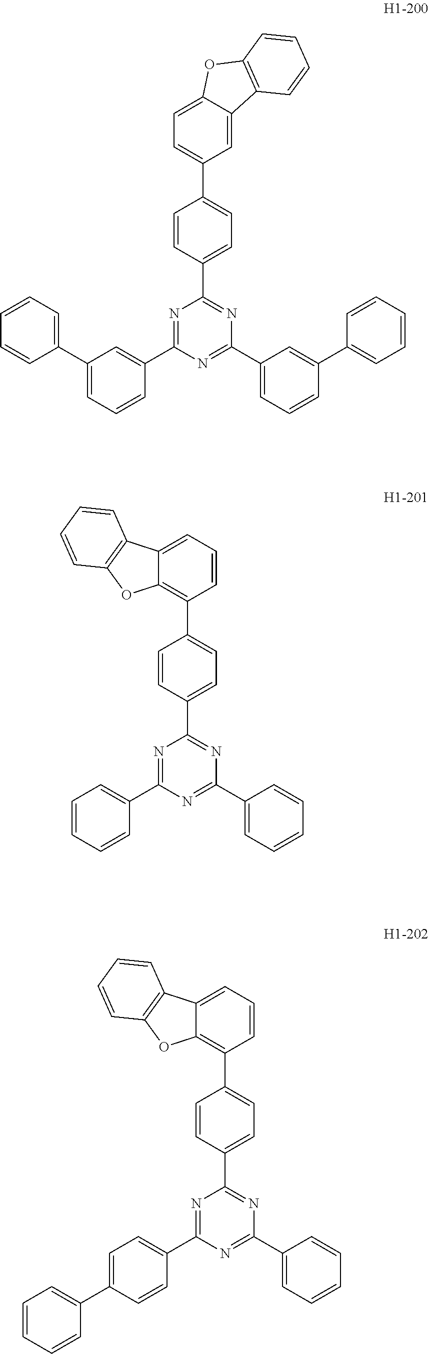 [SMILES: c1ccc(-c2cccc(-c3nc(-c4ccc(-c5ccc6oc7ccccc7c6c5)cc4)nc(-c4cccc(-c5ccccc5)c4)n3)c2)cc1 ; c1ccc(-c2nc(-c3ccccc3)nc(-c3ccc(-c4cccc5c4oc4ccccc45)cc3)n2)cc1 ; c1ccc(-c2ccc(-c3nc(-c4ccccc4)nc(-c4ccc(-c5cccc6c5oc5ccccc56)cc4)n3)cc2)cc1]

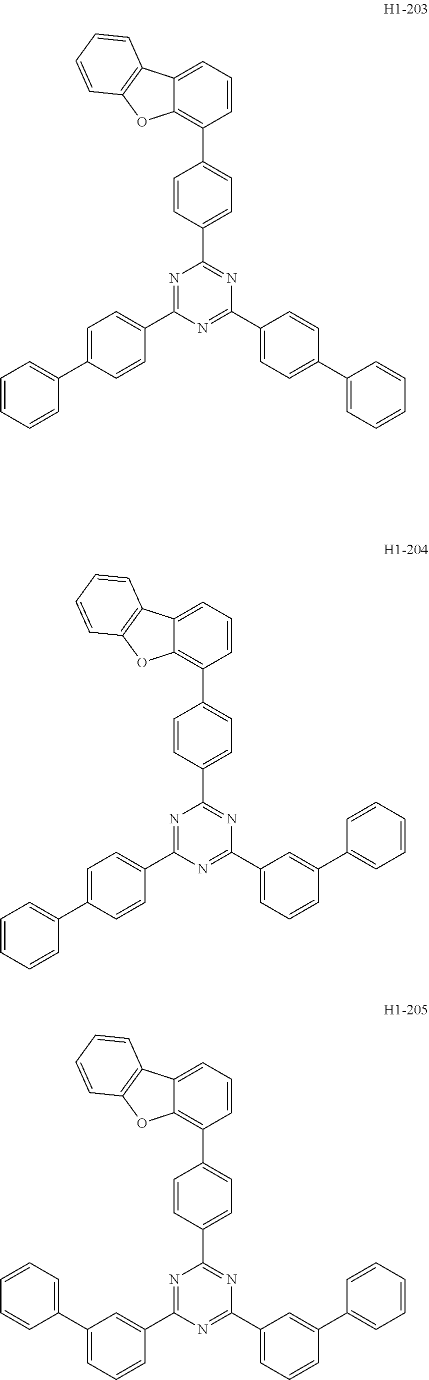 [SMILES: c1ccc(-c2ccc(-c3nc(-c4ccc(-c5ccccc5)cc4)nc(-c4ccc(-c5cccc6c5oc5ccccc56)cc4)n3)cc2)cc1 ; c1ccc(-c2ccc(-c3nc(-c4ccc(-c5cccc6c5oc5ccccc56)cc4)nc(-c4cccc(-c5ccccc5)c4)n3)cc2)cc1 ; c1ccc(-c2cccc(-c3nc(-c4ccc(-c5cccc6c5oc5ccccc56)cc4)nc(-c4cccc(-c5ccccc5)c4)n3)c2)cc1]

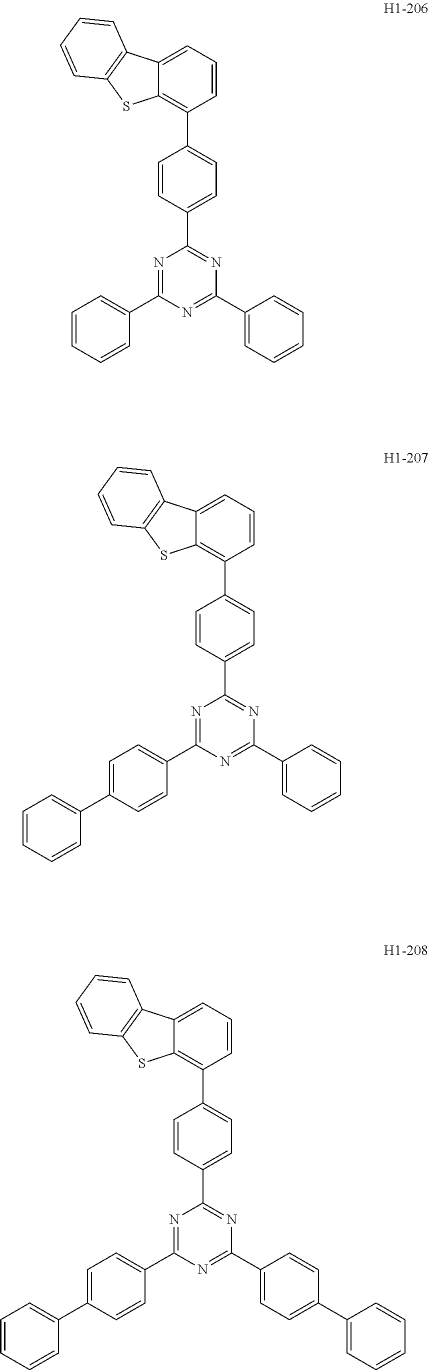 [SMILES: c1ccc(-c2nc(-c3ccccc3)nc(-c3ccc(-c4cccc5c4sc4ccccc45)cc3)n2)cc1 ; c1ccc(-c2ccc(-c3nc(-c4ccccc4)nc(-c4ccc(-c5cccc6c5sc5ccccc56)cc4)n3)cc2)cc1 ; c1ccc(-c2ccc(-c3nc(-c4ccc(-c5ccccc5)cc4)nc(-c4ccc(-c5cccc6c5sc5ccccc56)cc4)n3)cc2)cc1]

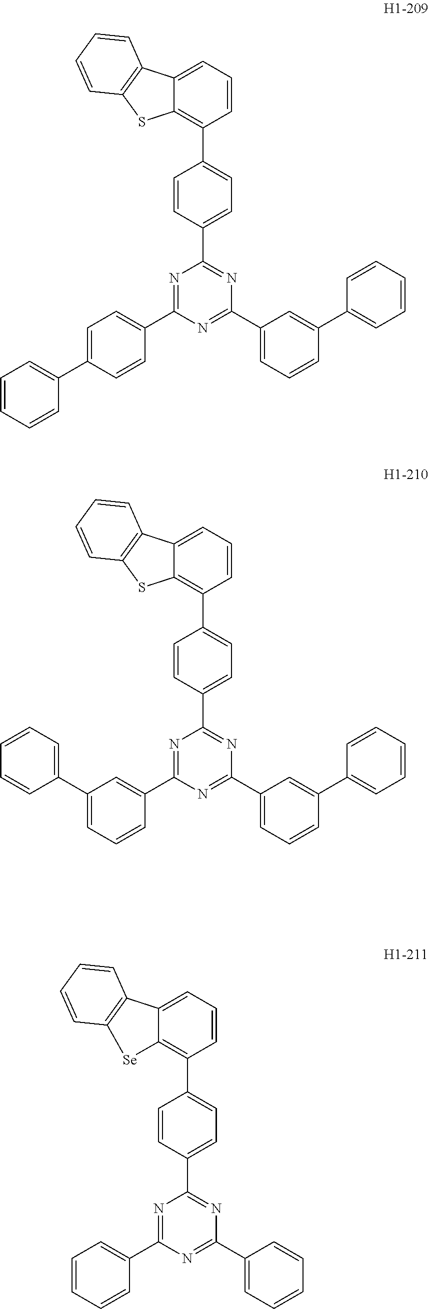 [SMILES: c1ccc(-c2ccc(-c3nc(-c4ccc(-c5cccc6c5sc5ccccc56)cc4)nc(-c4cccc(-c5ccccc5)c4)n3)cc2)cc1 ; c1ccc(-c2cccc(-c3nc(-c4ccc(-c5cccc6c5sc5ccccc56)cc4)nc(-c4cccc(-c5ccccc5)c4)n3)c2)cc1 ; c1ccc(-c2nc(-c3ccccc3)nc(-c3ccc(-c4cccc5c4[se]c4ccccc45)cc3)n2)cc1]

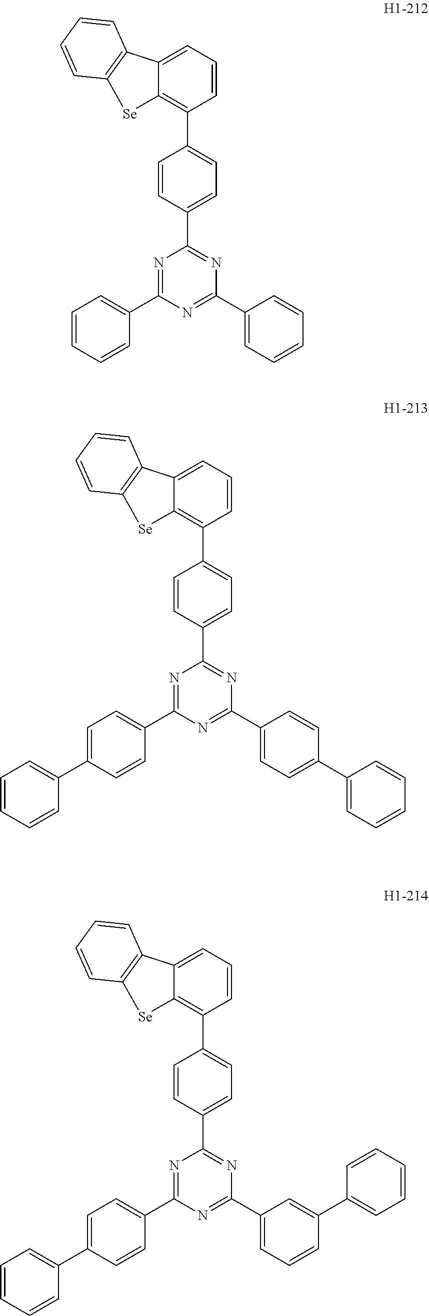 [SMILES: c1ccc(-c2nc(-c3ccccc3)nc(-c3ccc(-c4cccc5c4[se]c4ccccc45)cc3)n2)cc1 ; c1ccc(-c2ccc(-c3nc(-c4ccc(-c5ccccc5)cc4)nc(-c4ccc(-c5cccc6c5[se]c5ccccc56)cc4)n3)cc2)cc1 ; c1ccc(-c2ccc(-c3nc(-c4ccc(-c5cccc6c5[se]c5ccccc56)cc4)nc(-c4cccc(-c5ccccc5)c4)n3)cc2)cc1]

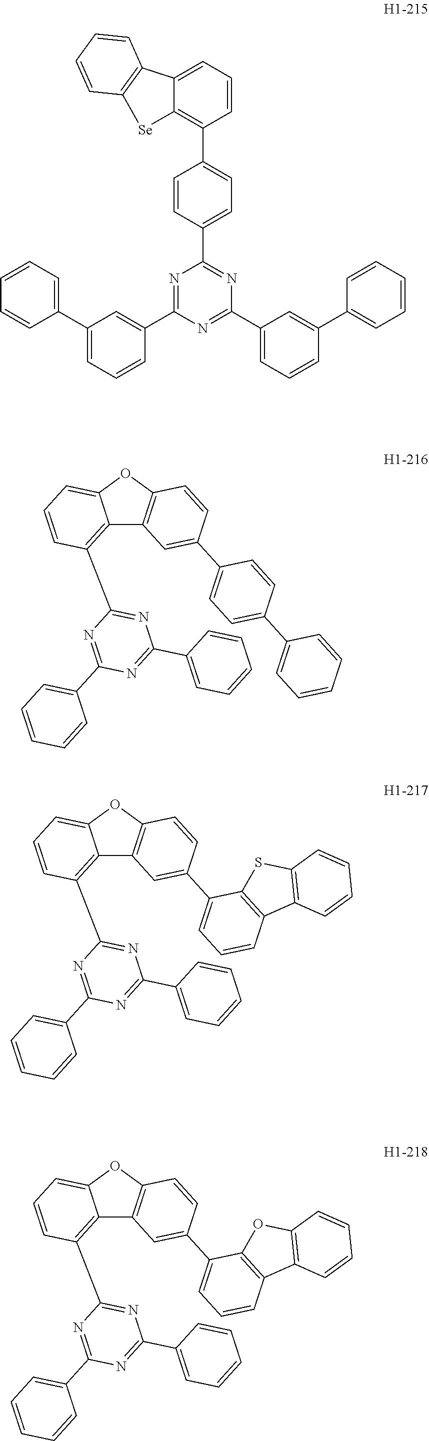 [SMILES: c1ccc(-c2cccc(-c3nc(-c4ccc(-c5cccc6c5[se]c5ccccc56)cc4)nc(-c4cccc(-c5ccccc5)c4)n3)c2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2nc(-c3ccccc3)nc(-c3cccc4oc5ccc(-c6cccc7c6sc6ccccc67)cc5c34)n2)cc1 ; c1ccc(-c2nc(-c3ccccc3)nc(-c3cccc4oc5ccc(-c6cccc7c6oc6ccccc67)cc5c34)n2)cc1]

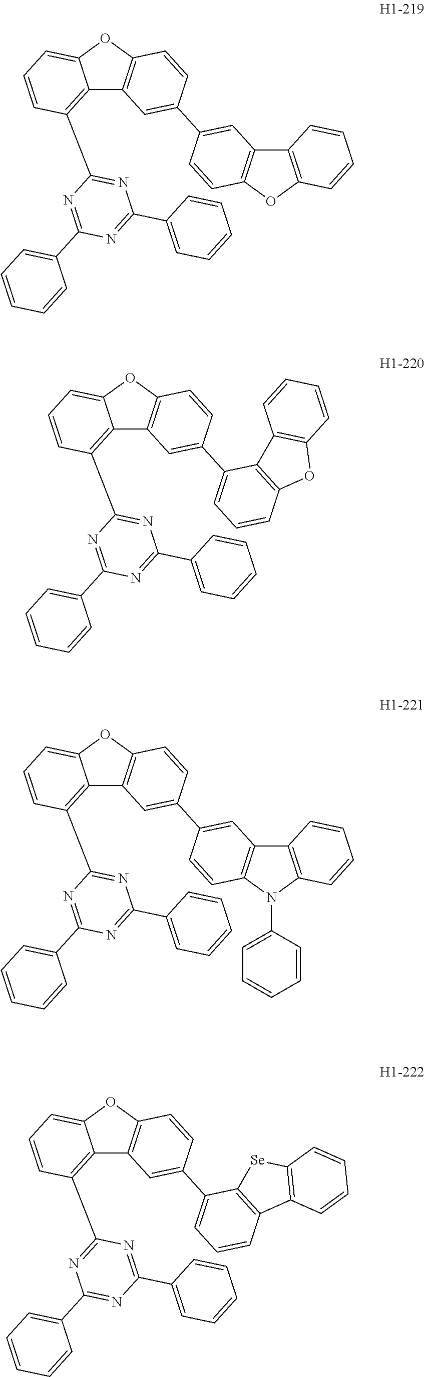 [SMILES: c1ccc(-c2nc(-c3ccccc3)nc(-c3cccc4oc5ccc(-c6ccc7oc8ccccc8c7c6)cc5c34)n2)cc1 ; c1ccc(-c2nc(-c3ccccc3)nc(-c3cccc4oc5ccc(-c6cccc7oc8ccccc8c67)cc5c34)n2)cc1 ; c1ccc(-c2nc(-c3ccccc3)nc(-c3cccc4oc5ccc(-c6ccc7c(c6)c6ccccc6n7-c6ccccc6)cc5c34)n2)cc1 ; c1ccc(-c2nc(-c3ccccc3)nc(-c3cccc4oc5ccc(-c6cccc7c6[se]c6ccccc67)cc5c34)n2)cc1]

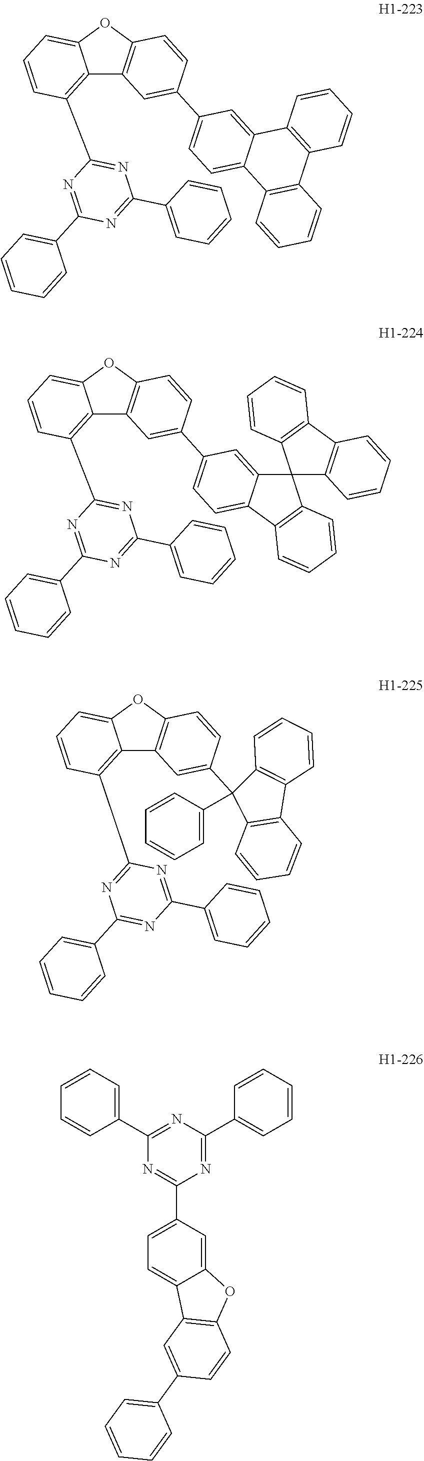 [SMILES: c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1 ; c1ccc(-c2nc(-c3ccccc3)nc(-c3cccc4oc5ccc(C6(c7ccccc7)c7ccccc7-c7ccccc76)cc5c34)n2)cc1 ; c1ccc(-c2ccc3oc4cc(-c5nc(-c6ccccc6)nc(-c6ccccc6)n5)ccc4c3c2)cc1]

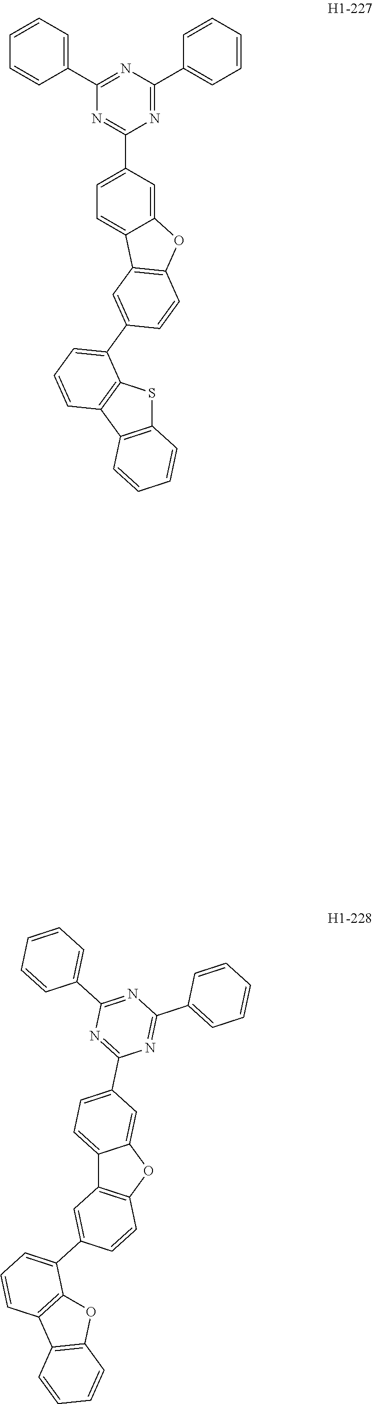 [SMILES: c1ccc(-c2nc(-c3ccccc3)nc(-c3ccc4c(c3)oc3ccc(-c5cccc6c5sc5ccccc56)cc34)n2)cc1 ; c1ccc(-c2nc(-c3ccccc3)nc(-c3ccc4c(c3)oc3ccc(-c5cccc6c5oc5ccccc56)cc34)n2)cc1]

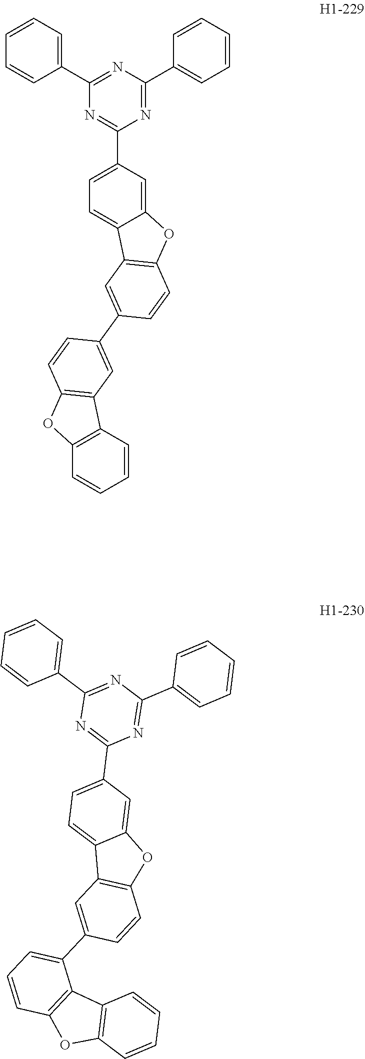 [SMILES: c1ccc(-c2nc(-c3ccccc3)nc(-c3ccc4c(c3)oc3ccc(-c5ccc6oc7ccccc7c6c5)cc34)n2)cc1 ; c1ccc(-c2nc(-c3ccccc3)nc(-c3ccc4c(c3)oc3ccc(-c5cccc6oc7ccccc7c56)cc34)n2)cc1]

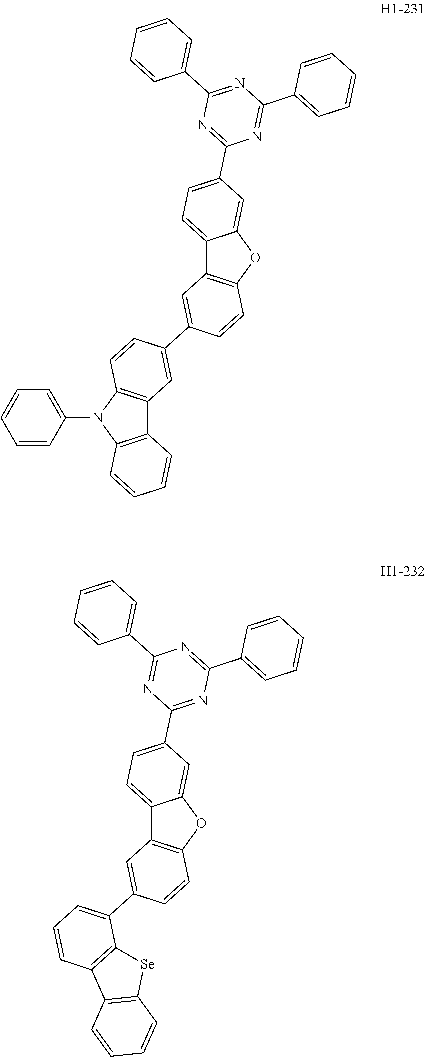 [SMILES: c1ccc(-c2nc(-c3ccccc3)nc(-c3ccc4c(c3)oc3ccc(-c5ccc6c(c5)c5ccccc5n6-c5ccccc5)cc34)n2)cc1 ; c1ccc(-c2nc(-c3ccccc3)nc(-c3ccc4c(c3)oc3ccc(-c5cccc6c5[se]c5ccccc56)cc34)n2)cc1]

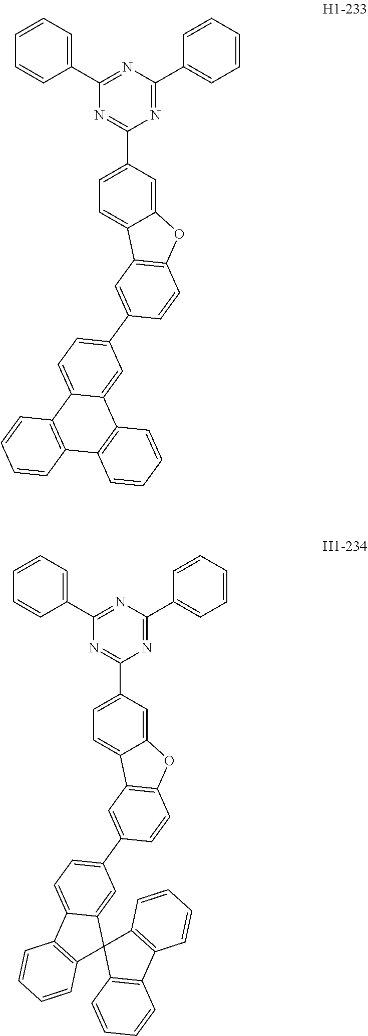 [SMILES: c1ccc(-c2nc(-c3ccccc3)nc(-c3ccc4c(c3)oc3ccc(-c5ccc6c7ccccc7c7ccccc7c6c5)cc34)n2)cc1 ; c1ccc(-c2nc(-c3ccccc3)nc(-c3ccc4c(c3)oc3ccc(-c5ccc6c(c5)C5(c7ccccc7-c7ccccc75)c5ccccc5-6)cc34)n2)cc1]

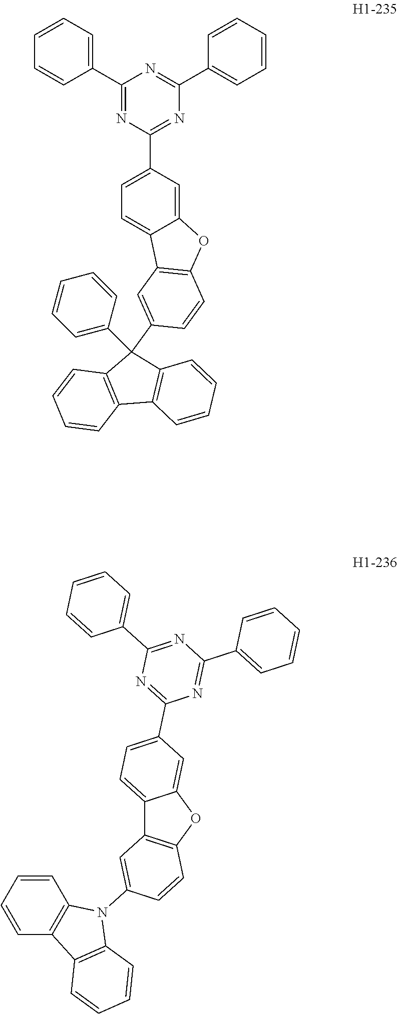 [SMILES: c1ccc(-c2nc(-c3ccccc3)nc(-c3ccc4c(c3)oc3ccc(C5(c6ccccc6)c6ccccc6-c6ccccc65)cc34)n2)cc1 ; c1ccc(-c2nc(-c3ccccc3)nc(-c3ccc4c(c3)oc3ccc(-n5c6ccccc6c6ccccc65)cc34)n2)cc1]

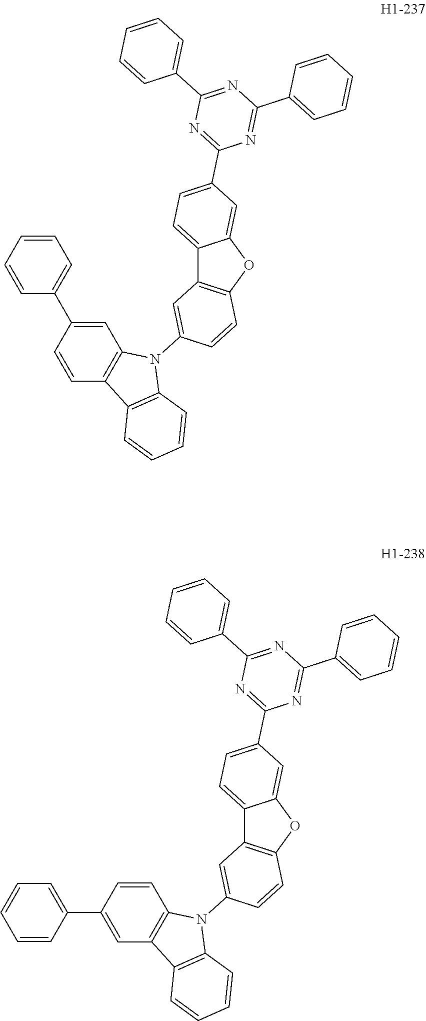 [SMILES: c1ccc(-c2ccc3c4ccccc4n(-c4ccc5oc6cc(-c7nc(-c8ccccc8)nc(-c8ccccc8)n7)ccc6c5c4)c3c2)cc1 ; c1ccc(-c2ccc3c(c2)c2ccccc2n3-c2ccc3oc4cc(-c5nc(-c6ccccc6)nc(-c6ccccc6)n5)ccc4c3c2)cc1]

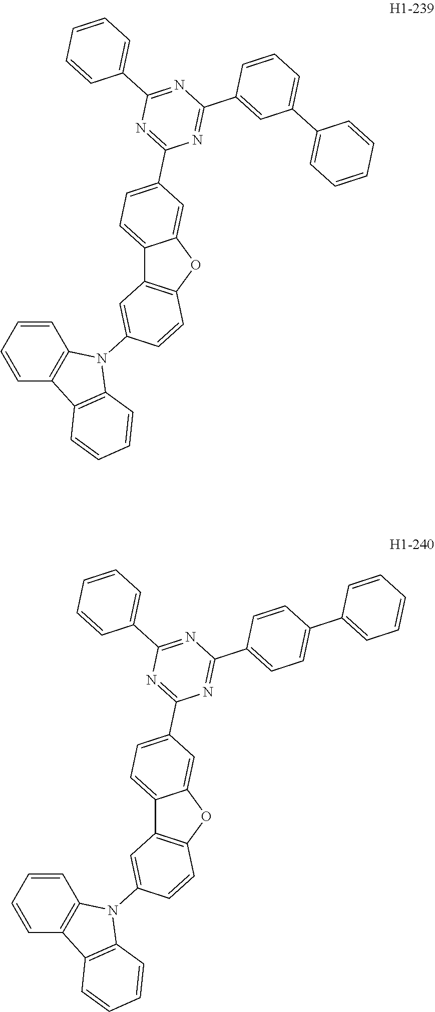 [SMILES: c1ccc(-c2cccc(-c3nc(-c4ccccc4)nc(-c4ccc5c(c4)oc4ccc(-n6c7ccccc7c7ccccc76)cc45)n3)c2)cc1 ; c1ccc(-c2ccc(-c3nc(-c4ccccc4)nc(-c4ccc5c(c4)oc4ccc(-n6c7ccccc7c7ccccc76)cc45)n3)cc2)cc1]

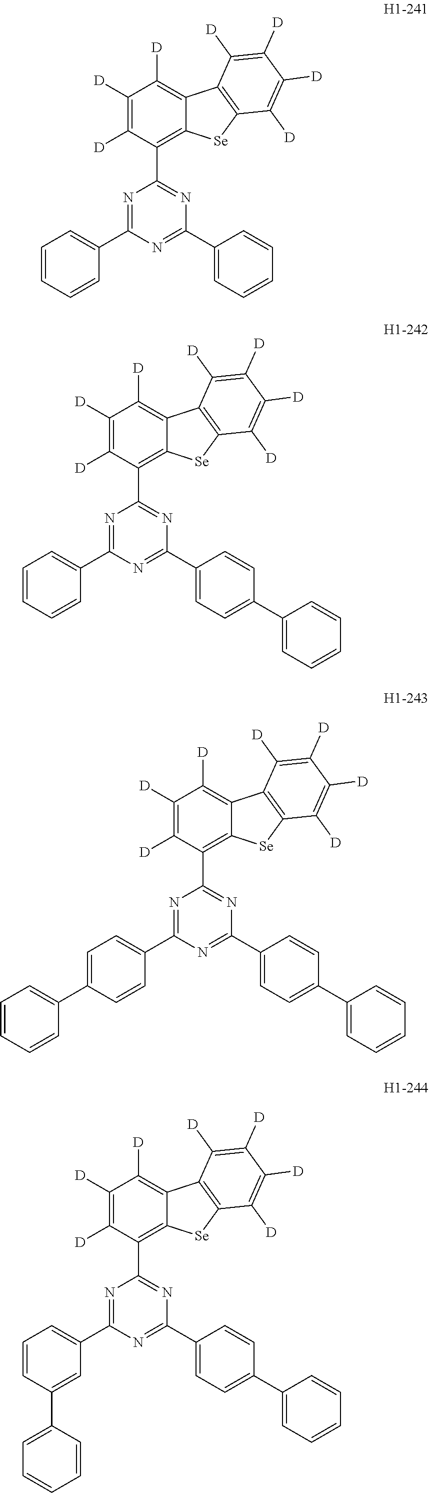 [SMILES: [2H]c1c([2H])c([2H])c2c([se]c3c(-c4nc(-c5ccccc5)nc(-c5ccccc5)n4)c([2H])c([2H])c([2H])c32)c1[2H] ; [2H]c1c([2H])c([2H])c2c([se]c3c(-c4nc(-c5ccccc5)nc(-c5ccc(-c6ccccc6)cc5)n4)c([2H])c([2H])c([2H])c32)c1[2H] ; [2H]c1c([2H])c([2H])c2c([se]c3c(-c4nc(-c5ccc(-c6ccccc6)cc5)nc(-c5ccc(-c6ccccc6)cc5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6ccccc6)c5)n4)c([2H])c([2H])c([2H])c32)c1[2H]]

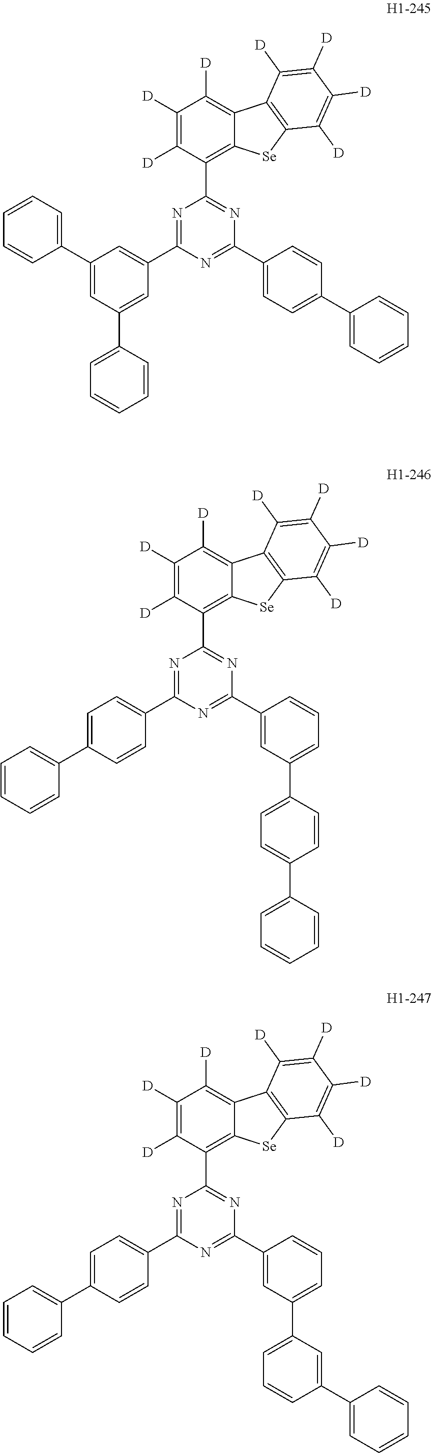 [SMILES: [2H]c1c([2H])c([2H])c2c([se]c3c(-c4nc(-c5ccc(-c6ccccc6)cc5)nc(-c5cc(-c6ccccc6)cc(-c6ccccc6)c5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6ccc(-c7ccccc7)cc6)c5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6cccc(-c7ccccc7)c6)c5)n4)c([2H])c([2H])c([2H])c32)c1[2H]]

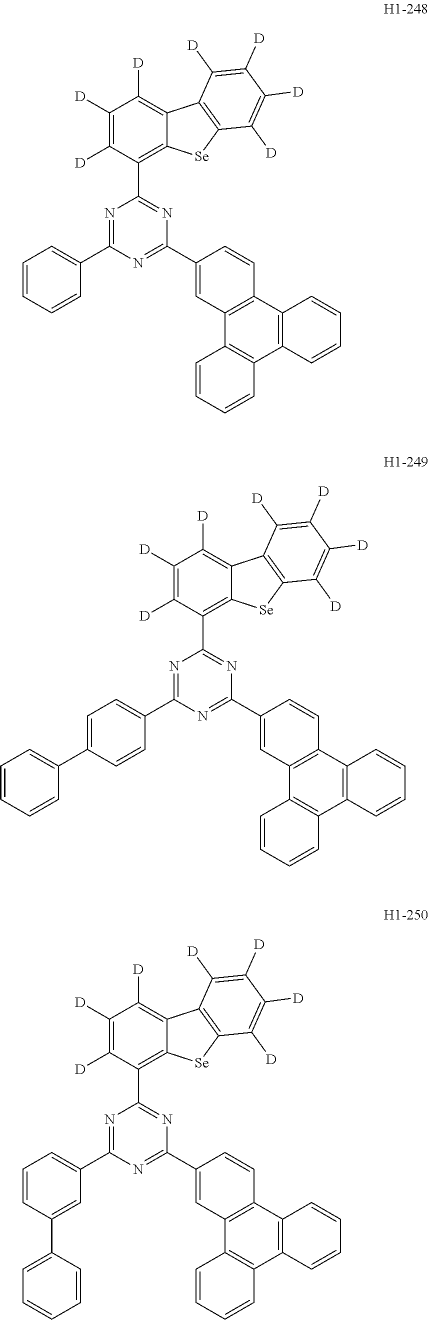 [SMILES: [2H]c1c([2H])c([2H])c2c([se]c3c(-c4nc(-c5ccccc5)nc(-c5ccc6c7ccccc7c7ccccc7c6c5)n4)c([2H])c([2H])c([2H])c32)c1[2H] ; [2H]c1c([2H])c([2H])c2c([se]c3c(-c4nc(-c5ccc(-c6ccccc6)cc5)nc(-c5ccc6c7ccccc7c7ccccc7c6c5)n4)c([2H])c([2H])c([2H])c32)c1[2H] ; [2H]c1c([2H])c([2H])c2c([se]c3c(-c4nc(-c5cccc(-c6ccccc6)c5)nc(-c5ccc6c7ccccc7c7ccccc7c6c5)n4)c([2H])c([2H])c([2H])c32)c1[2H]]

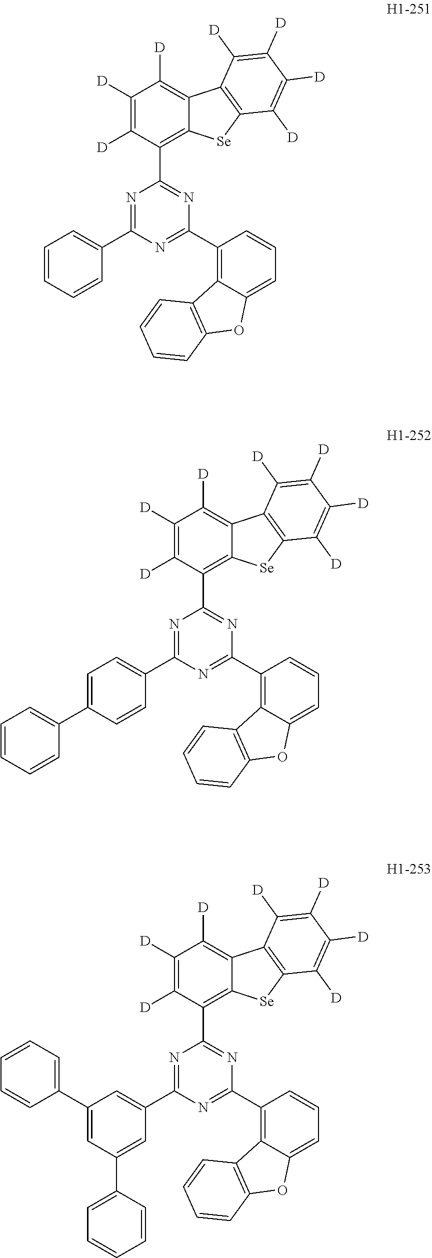 [SMILES: [2H]c1c([2H])c([2H])c2c([se]c3c(-c4nc(-c5ccccc5)nc(-c5cccc6oc7ccccc7c56)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc6oc7ccccc7c56)n4)c([2H])c([2H])c([2H])c32)c1[2H] ; [2H]c1c([2H])c([2H])c2c([se]c3c(-c4nc(-c5cc(-c6ccccc6)cc(-c6ccccc6)c5)nc(-c5cccc6oc7ccccc7c56)n4)c([2H])c([2H])c([2H])c32)c1[2H]]

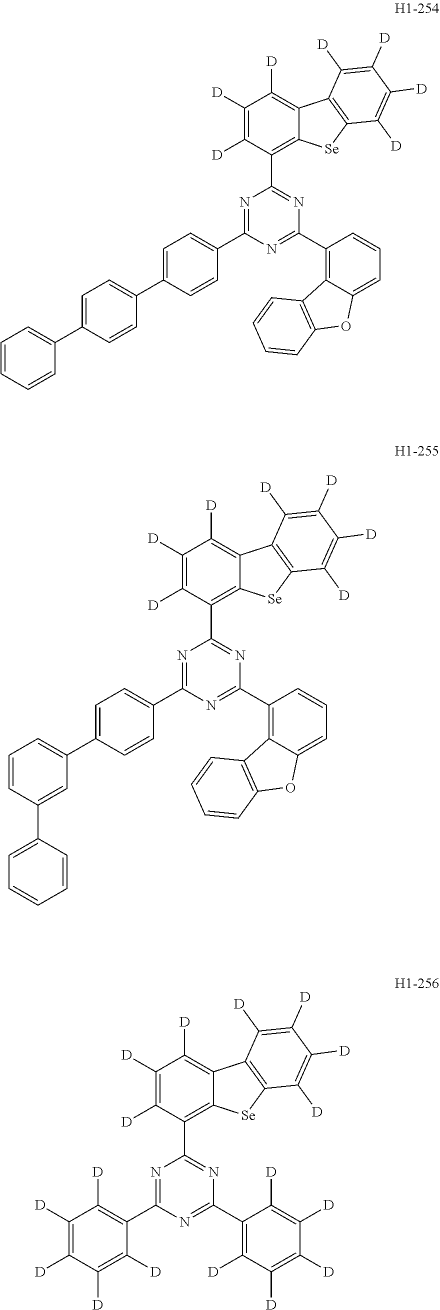 [SMILES: [2H]c1c([2H])c([2H])c2c([se]c3c(-c4nc(-c5ccc(-c6ccc(-c7ccccc7)cc6)cc5)nc(-c5cccc6oc7ccccc7c56)n4)c([2H])c([2H])c([2H])c32)c1[2H] ; [2H]c1c([2H])c([2H])c2c([se]c3c(-c4nc(-c5ccc(-c6cccc(-c7ccccc7)c6)cc5)nc(-c5cccc6oc7ccccc7c56)n4)c([2H])c([2H])c([2H])c32)c1[2H] ; [2H]c1c([2H])c([2H])c(-c2nc(-c3c([2H])c([2H])c([2H])c([2H])c3[2H])nc(-c3c([2H])c([2H])c([2H])c4c3[se]c3c([2H])c([2H])c([2H])c([2H])c34)n2)c([2H])c1[2H]]

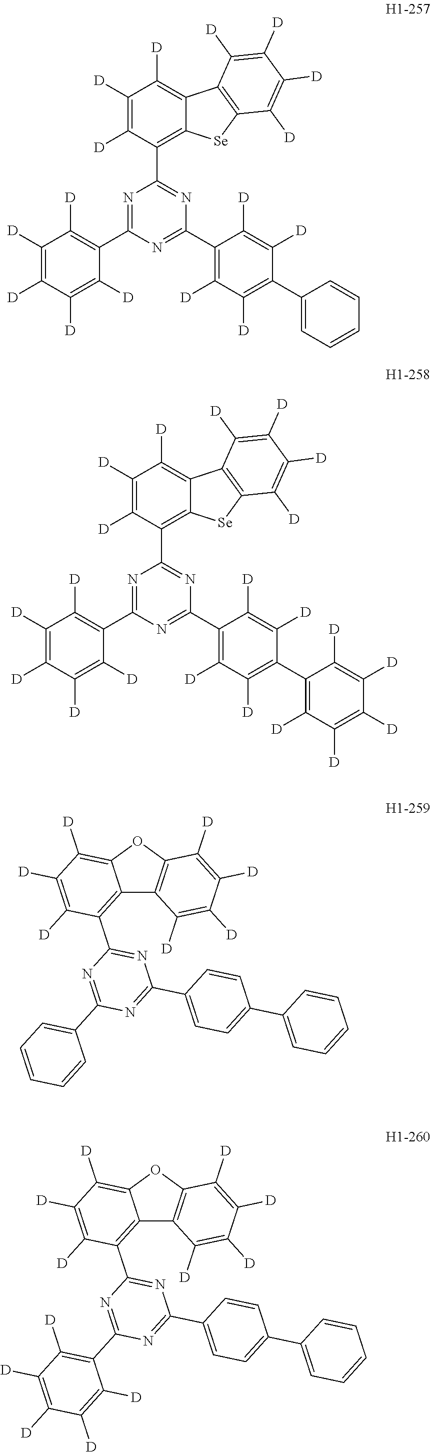

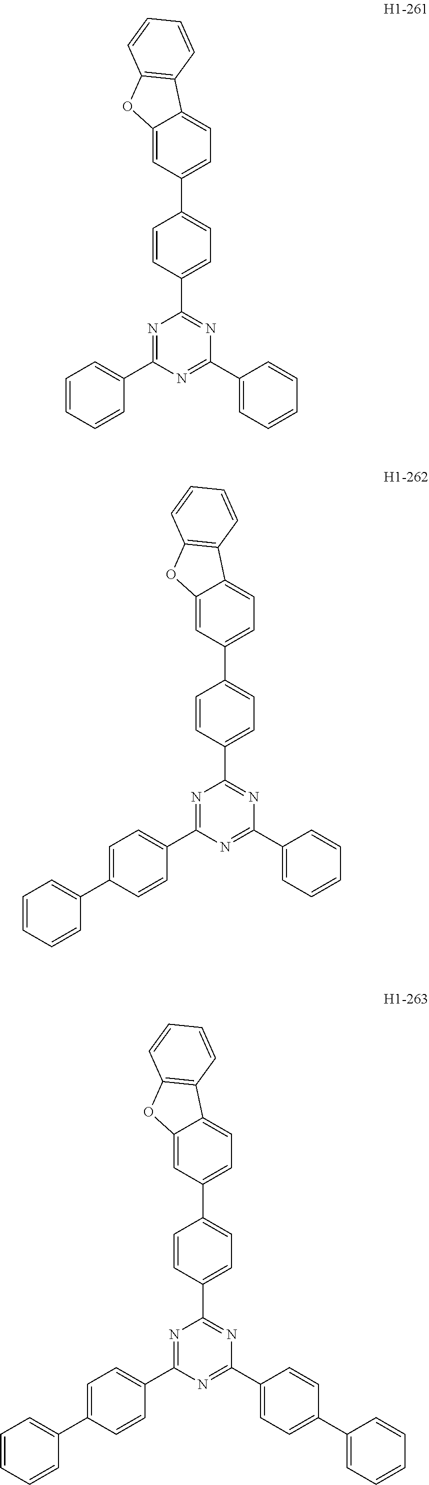 [SMILES: c1ccc(-c2nc(-c3ccccc3)nc(-c3ccc(-c4ccc5c(c4)oc4ccccc45)cc3)n2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3nc(-c4ccc(-c5ccccc5)cc4)nc(-c4ccc(-c5ccc6c(c5)oc5ccccc56)cc4)n3)cc2)cc1]

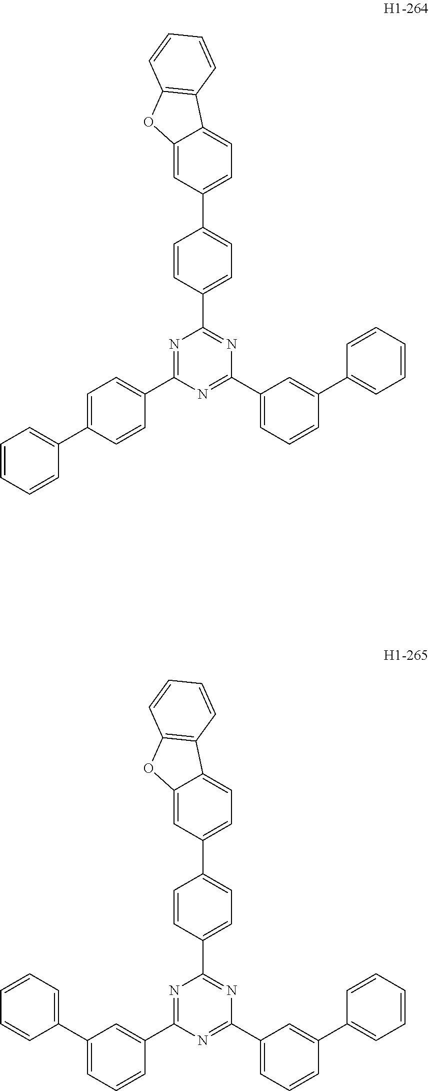 [SMILES: c1ccc(-c2ccc(-c3nc(-c4ccc(-c5ccc6c(c5)oc5ccccc56)cc4)nc(-c4cccc(-c5ccccc5)c4)n3)cc2)cc1 ; c1ccc(-c2cccc(-c3nc(-c4ccc(-c5ccc6c(c5)oc5ccccc56)cc4)nc(-c4cccc(-c5ccccc5)c4)n3)c2)cc1]

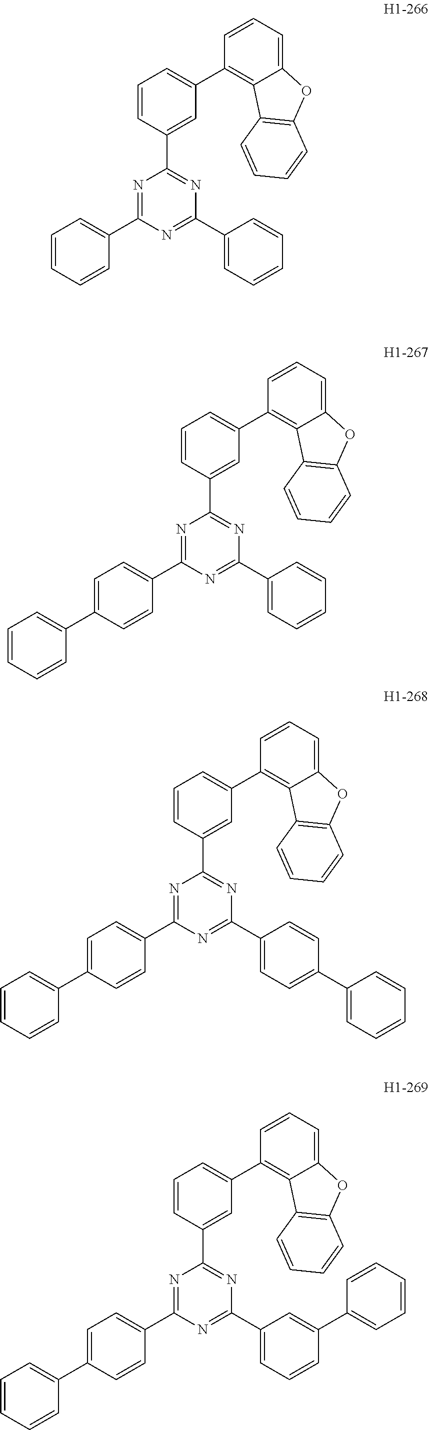 [SMILES: c1ccc(-c2nc(-c3ccccc3)nc(-c3cccc(-c4cccc5oc6ccccc6c45)c3)n2)cc1 ; c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4cccc(-c5ccccc5)c4)nc(-c4cccc(-c5cccc6oc7ccccc7c56)c4)n3)cc2)cc1]

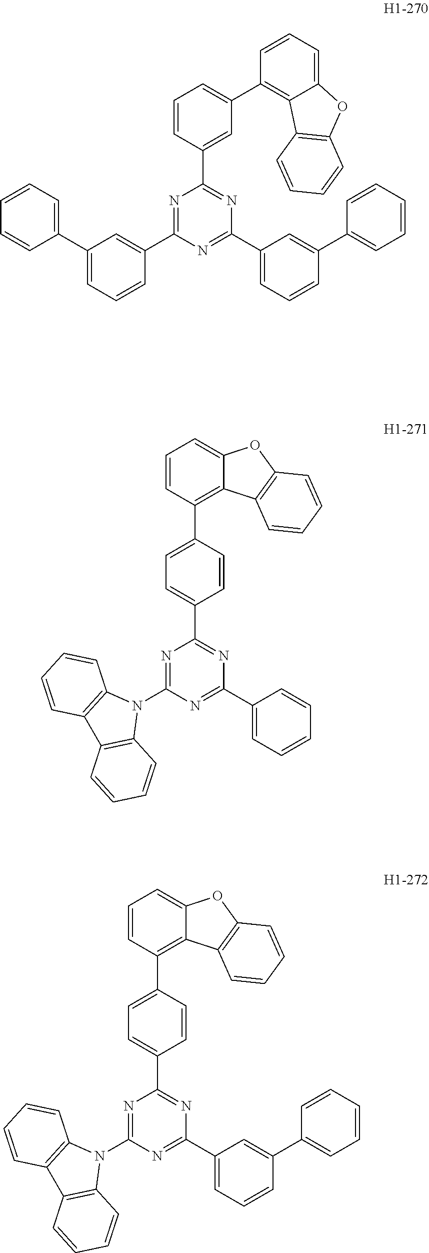 [SMILES: c1ccc(-c2cccc(-c3nc(-c4cccc(-c5ccccc5)c4)nc(-c4cccc(-c5cccc6oc7ccccc7c56)c4)n3)c2)cc1 ; c1ccc(-c2nc(-c3ccc(-c4cccc5oc6ccccc6c45)cc3)nc(-n3c4ccccc4c4ccccc43)n2)cc1 ; c1ccc(-c2cccc(-c3nc(-c4ccc(-c5cccc6oc7ccccc7c56)cc4)nc(-n4c5ccccc5c5ccccc54)n3)c2)cc1]

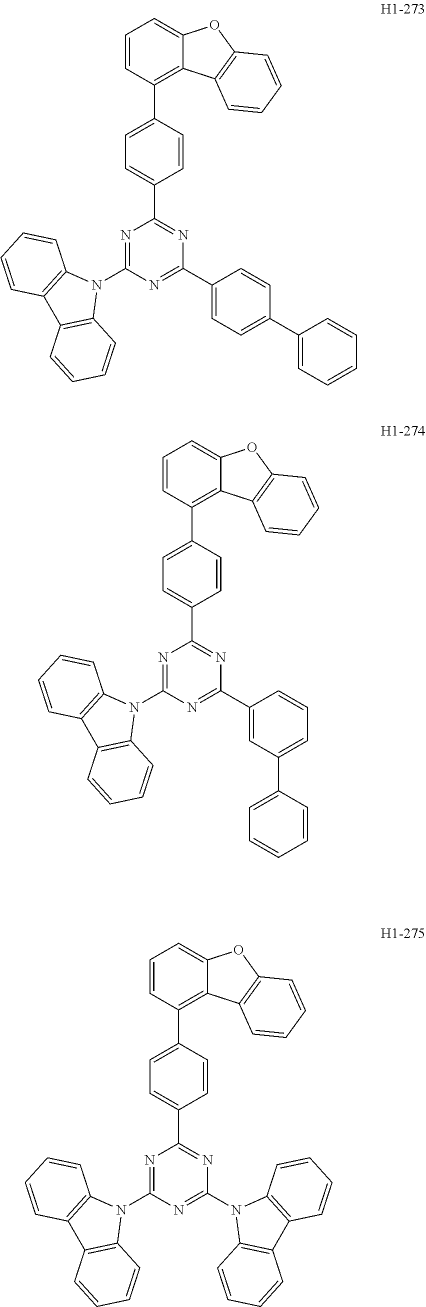 [SMILES: c1ccc(-c2ccc(-c3nc(-c4ccc(-c5cccc6oc7ccccc7c56)cc4)nc(-n4c5ccccc5c5ccccc54)n3)cc2)cc1 ; c1ccc(-c2cccc(-c3nc(-c4ccc(-c5cccc6oc7ccccc7c56)cc4)nc(-n4c5ccccc5c5ccccc54)n3)c2)cc1 ; c1ccc2c(c1)oc1cccc(-c3ccc(-c4nc(-n5c6ccccc6c6ccccc65)nc(-n5c6ccccc6c6ccccc65)n4)cc3)c12]

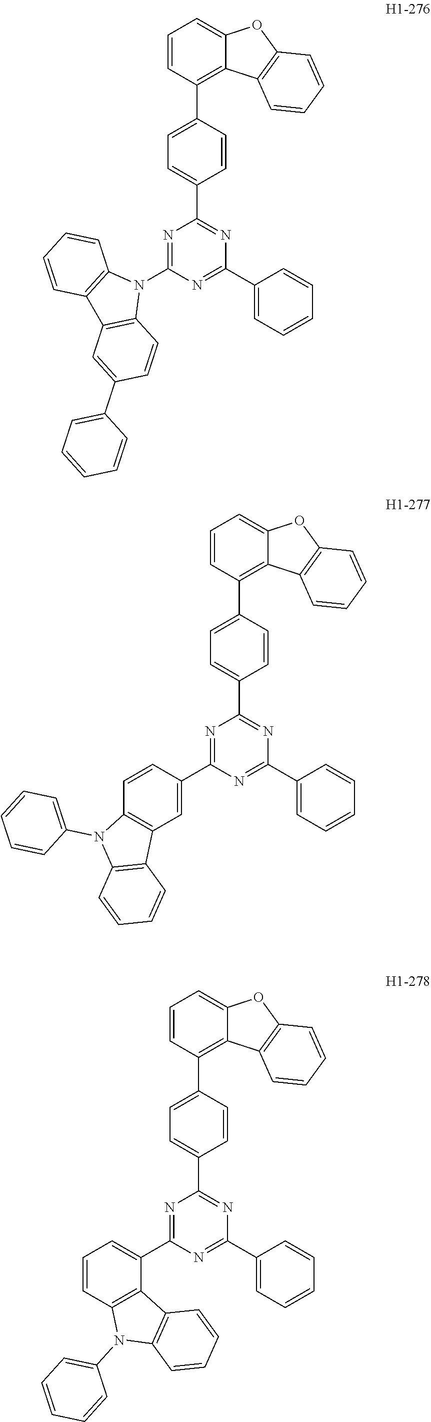 [SMILES: c1ccc(-c2ccc3c(c2)c2ccccc2n3-c2nc(-c3ccccc3)nc(-c3ccc(-c4cccc5oc6ccccc6c45)cc3)n2)cc1 ; c1ccc(-c2nc(-c3ccc(-c4cccc5oc6ccccc6c45)cc3)nc(-c3ccc4c(c3)c3ccccc3n4-c3ccccc3)n2)cc1 ; c1ccc(-c2nc(-c3ccc(-c4cccc5oc6ccccc6c45)cc3)nc(-c3cccc4c3c3ccccc3n4-c3ccccc3)n2)cc1]

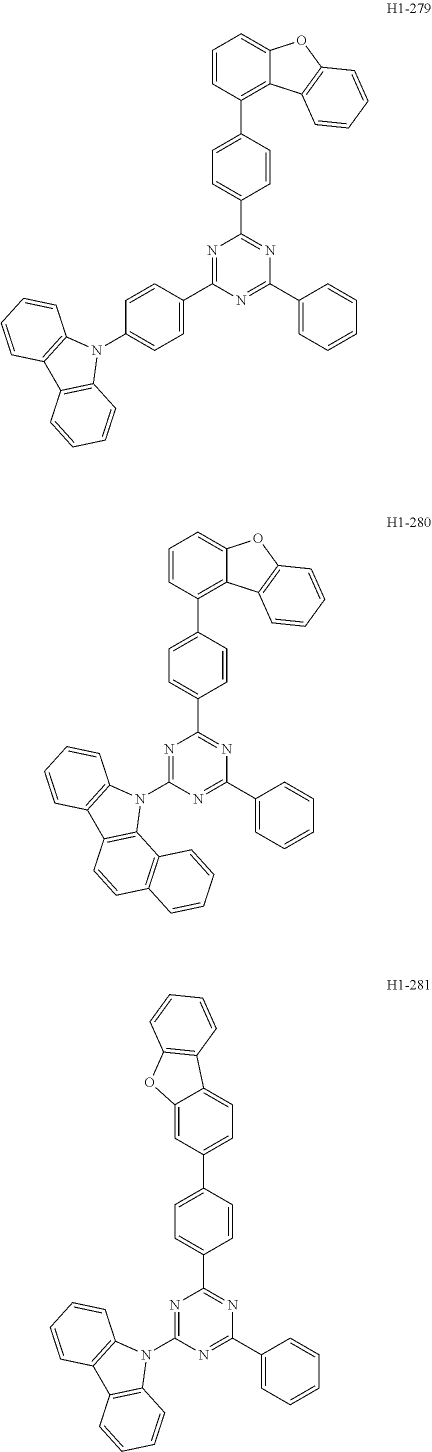 [SMILES: c1ccc(-c2nc(-c3ccc(-c4cccc5oc6ccccc6c45)cc3)nc(-c3ccc(-n4c5ccccc5c5ccccc54)cc3)n2)cc1 ; c1ccc(-c2nc(-c3ccc(-c4cccc5oc6ccccc6c45)cc3)nc(-n3c4ccccc4c4ccc5ccccc5c43)n2)cc1 ; c1ccc(-c2nc(-c3ccc(-c4ccc5c(c4)oc4ccccc45)cc3)nc(-n3c4ccccc4c4ccccc43)n2)cc1]

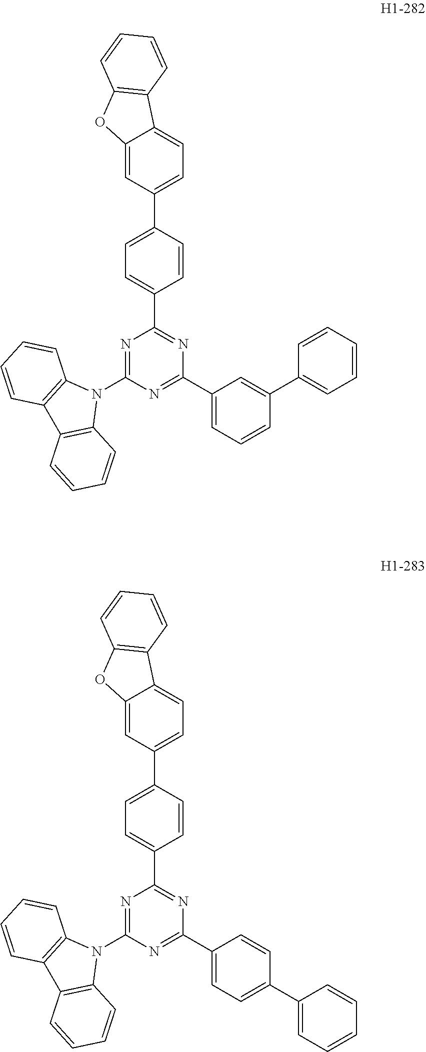 [SMILES: c1ccc(-c2cccc(-c3nc(-c4ccc(-c5ccc6c(c5)oc5ccccc56)cc4)nc(-n4c5ccccc5c5ccccc54)n3)c2)cc1 ; c1ccc(-c2ccc(-c3nc(-c4ccc(-c5ccc6c(c5)oc5ccccc56)cc4)nc(-n4c5ccccc5c5ccccc54)n3)cc2)cc1]

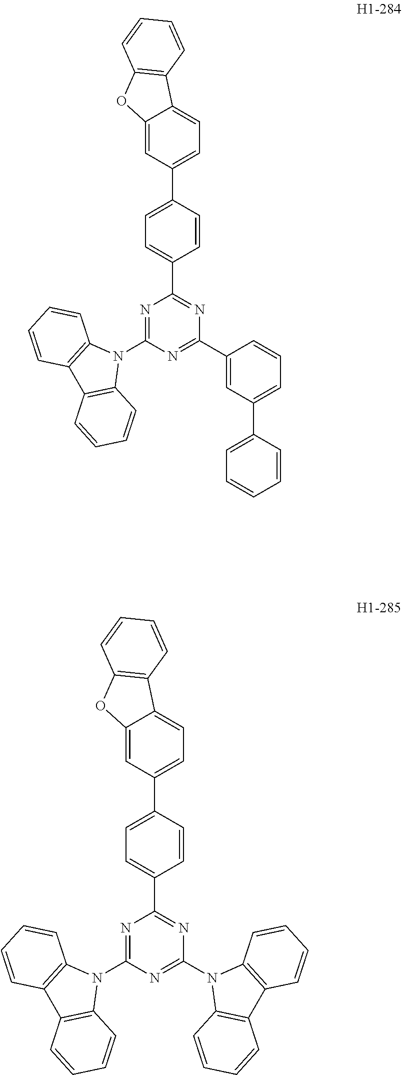 [SMILES: c1ccc(-c2cccc(-c3nc(-c4ccc(-c5ccc6c(c5)oc5ccccc56)cc4)nc(-n4c5ccccc5c5ccccc54)n3)c2)cc1 ; c1ccc2c(c1)oc1cc(-c3ccc(-c4nc(-n5c6ccccc6c6ccccc65)nc(-n5c6ccccc6c6ccccc65)n4)cc3)ccc12]

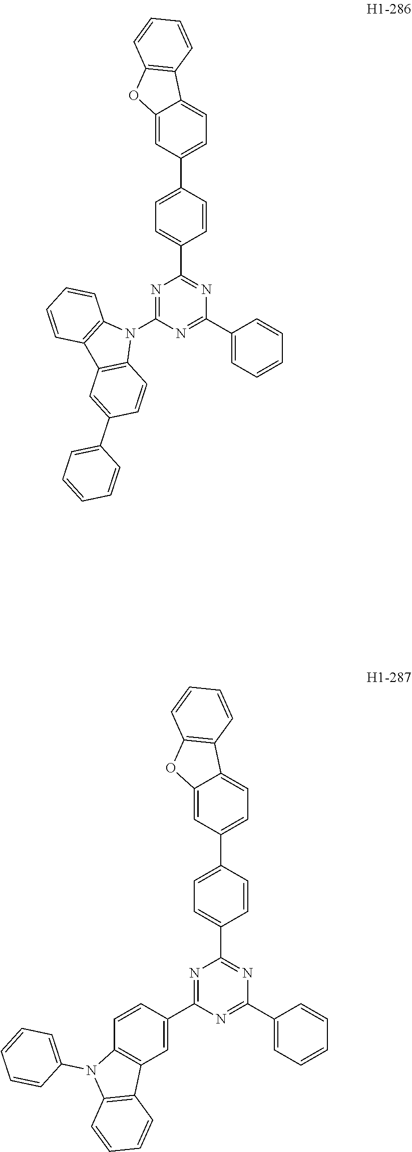 [SMILES: c1ccc(-c2ccc3c(c2)c2ccccc2n3-c2nc(-c3ccccc3)nc(-c3ccc(-c4ccc5c(c4)oc4ccccc45)cc3)n2)cc1 ; c1ccc(-c2nc(-c3ccc(-c4ccc5c(c4)oc4ccccc45)cc3)nc(-c3ccc4c(c3)c3ccccc3n4-c3ccccc3)n2)cc1]

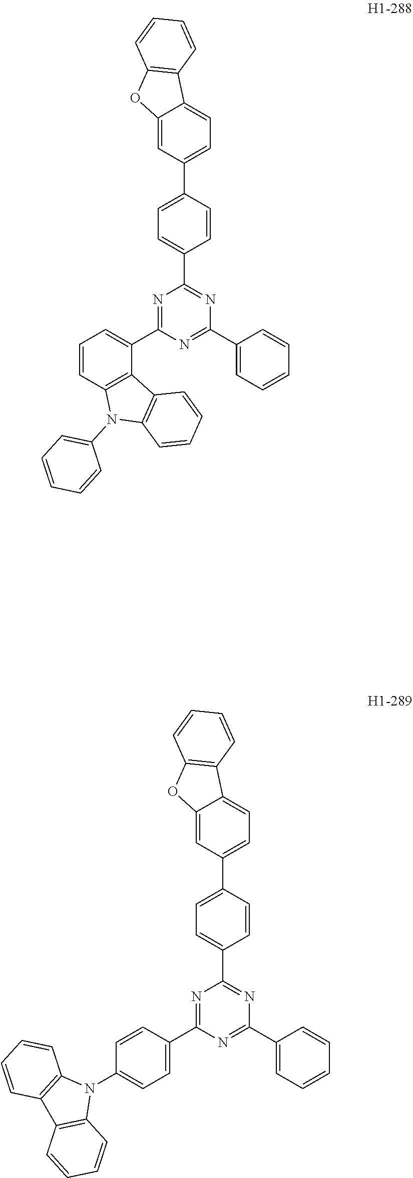 [SMILES: c1ccc(-c2nc(-c3ccc(-c4ccc5c(c4)oc4ccccc45)cc3)nc(-c3cccc4c3c3ccccc3n4-c3ccccc3)n2)cc1 ; c1ccc(-c2nc(-c3ccc(-c4ccc5c(c4)oc4ccccc45)cc3)nc(-c3ccc(-n4c5ccccc5c5ccccc54)cc3)n2)cc1]

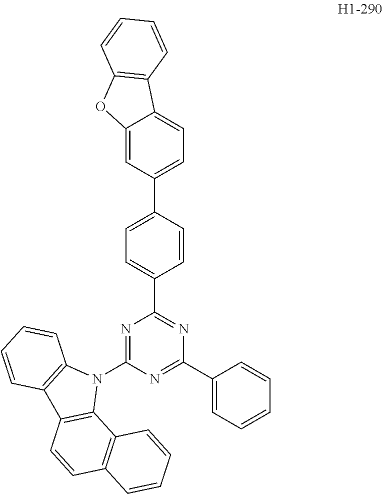 [SMILES: c1ccc(-c2nc(-c3ccc(-c4ccc5c(c4)oc4ccccc45)cc3)nc(-n3c4ccccc4c4ccc5ccccc5c43)n2)cc1]

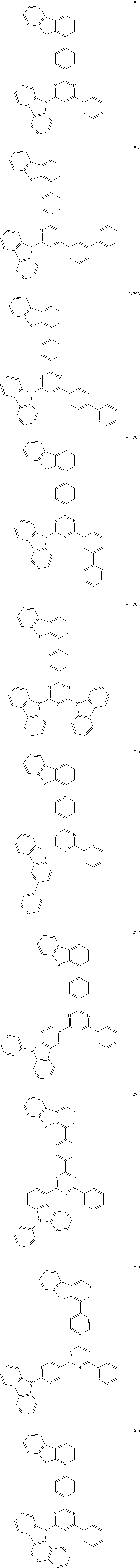

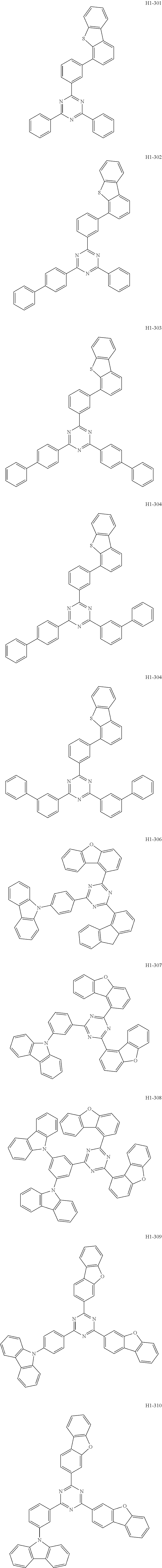

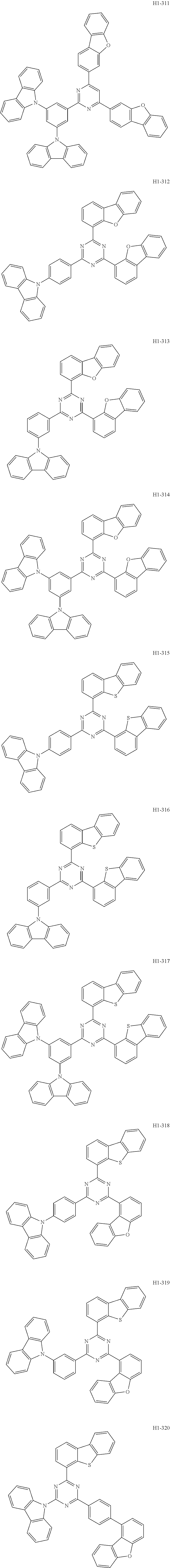

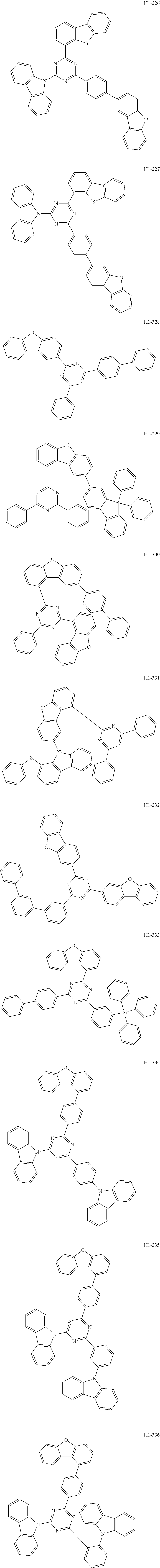

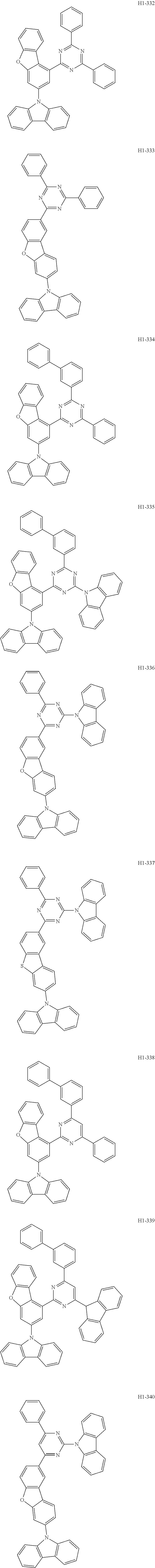

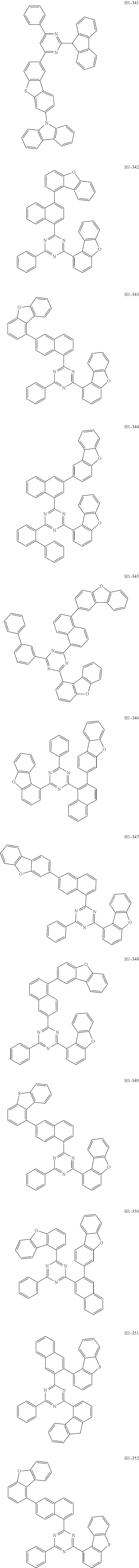

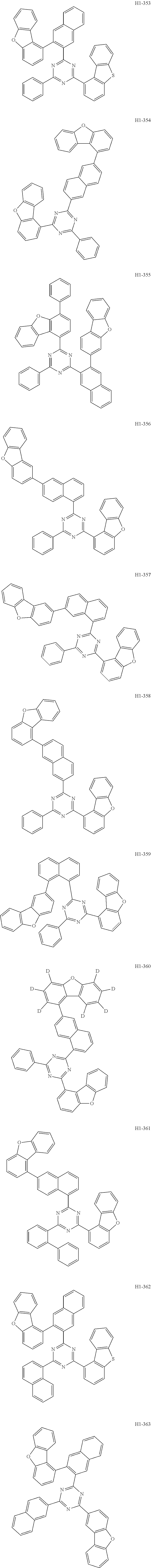

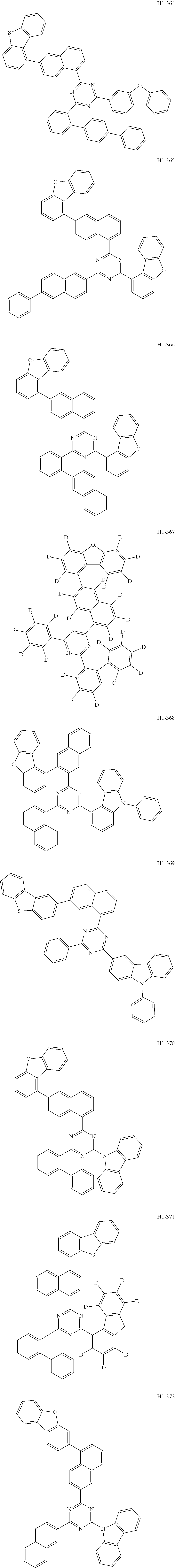

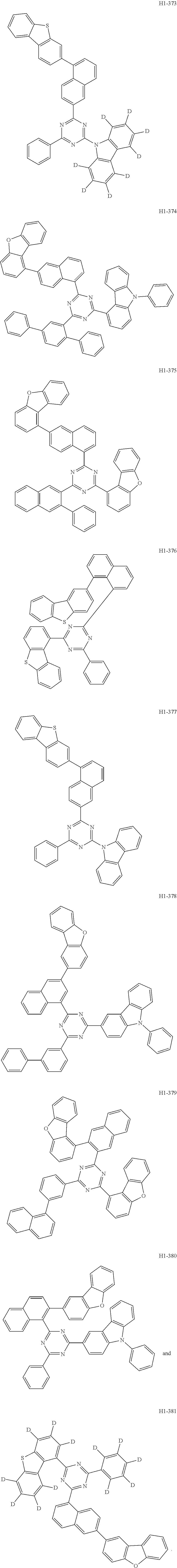

8. The plurality of host materials according to claim 1, wherein the compound represented by formula 2 is at least one selected from the group consisting of the following compounds: 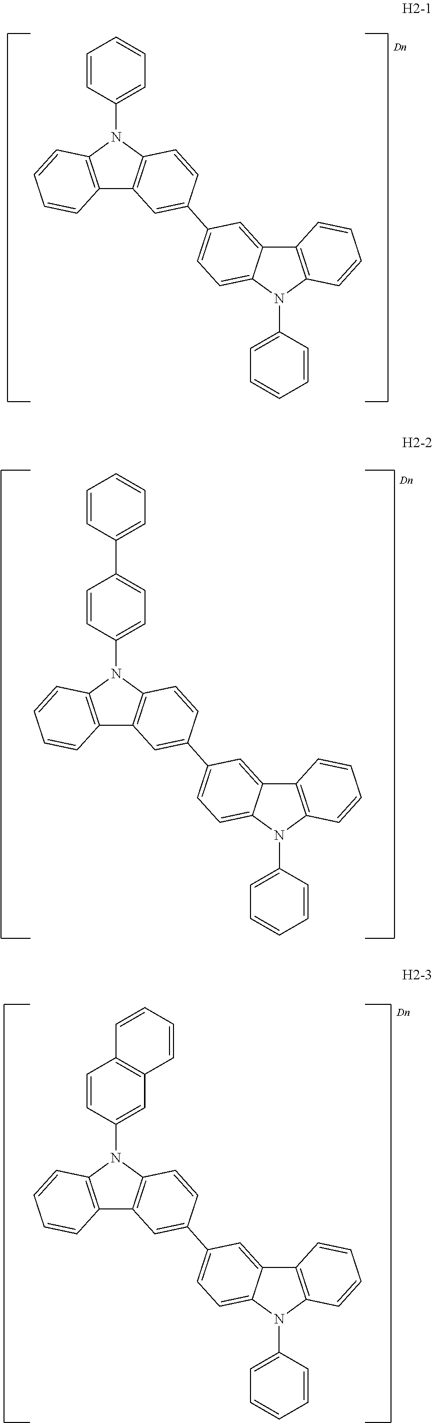 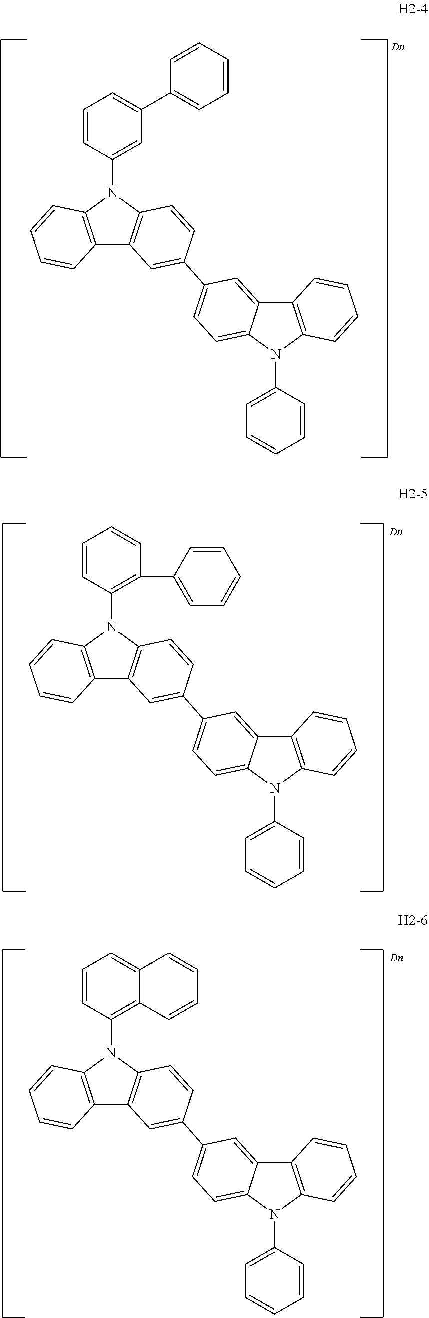 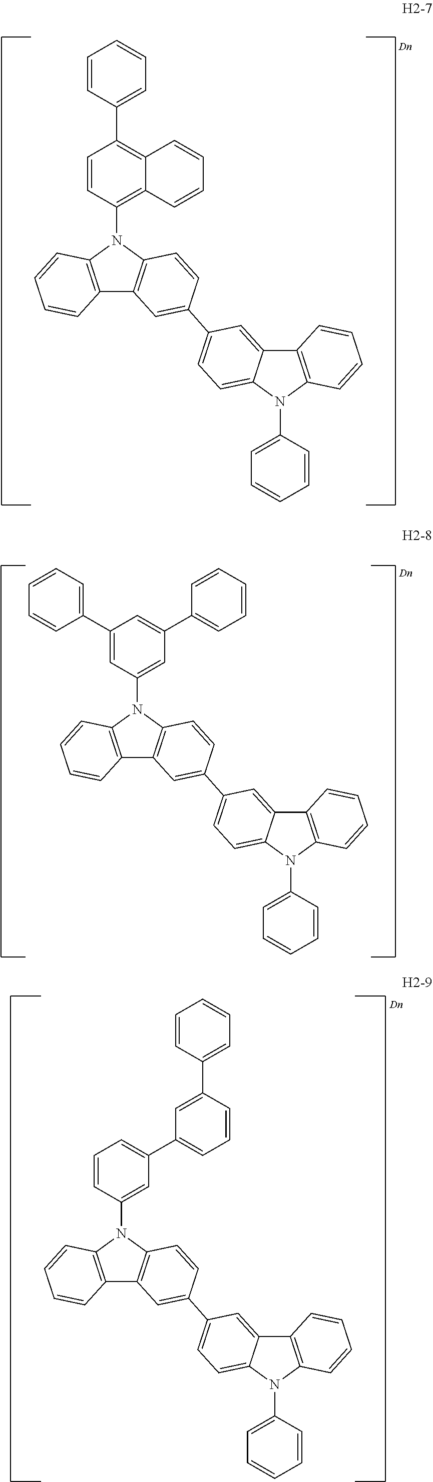 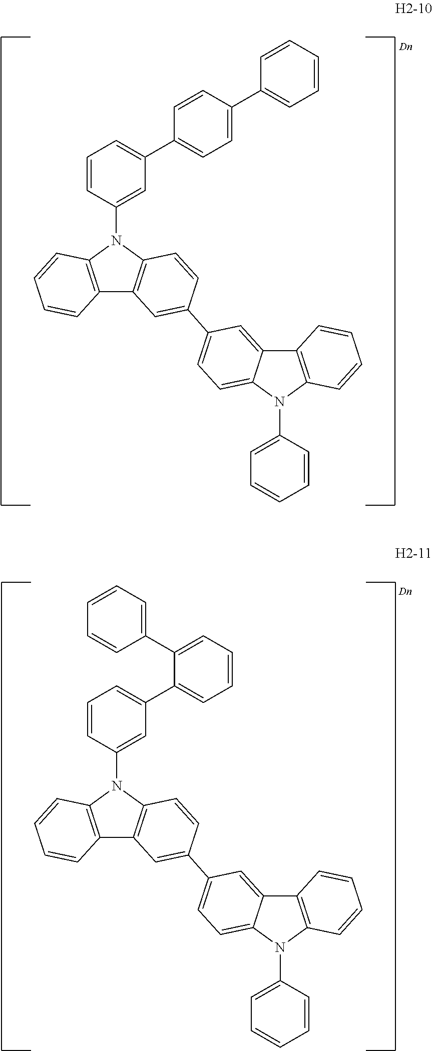 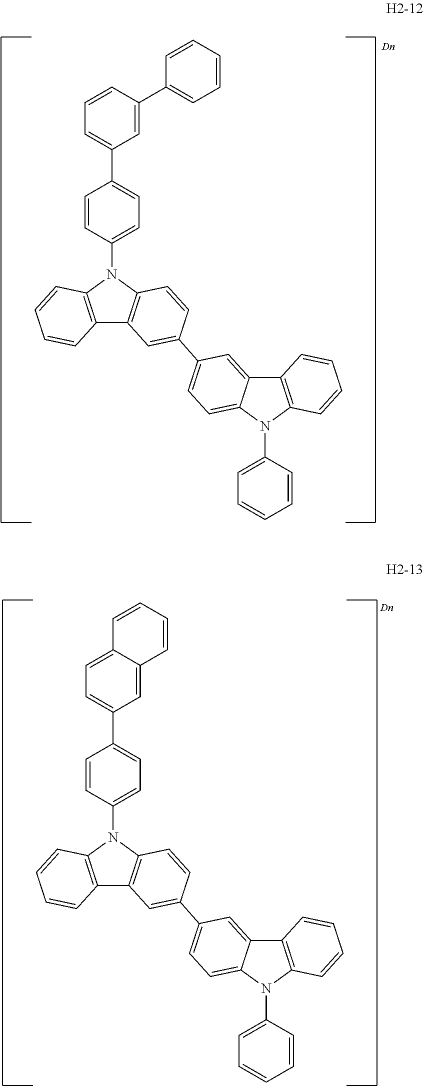 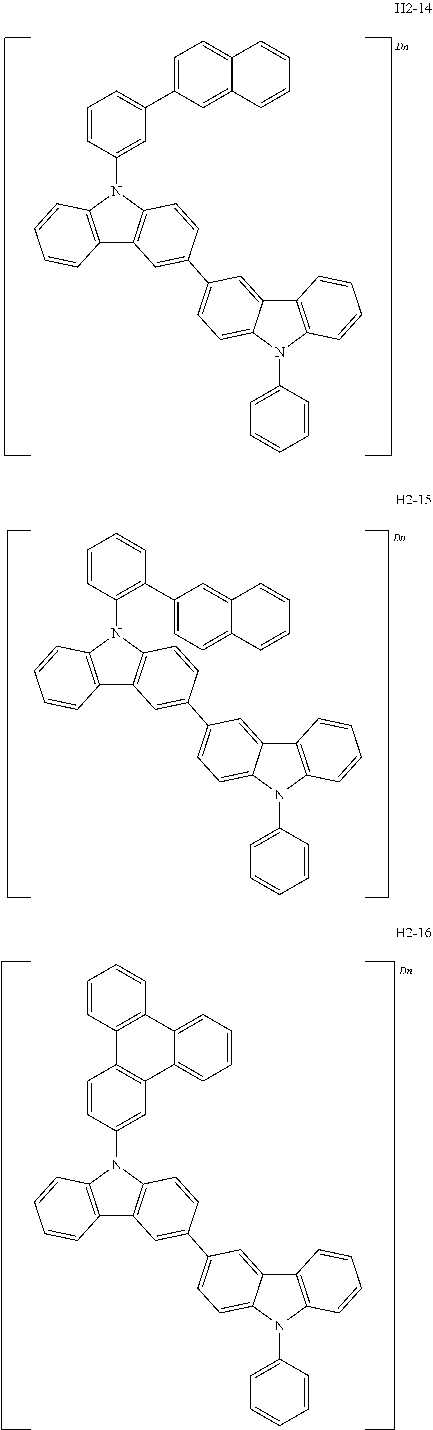 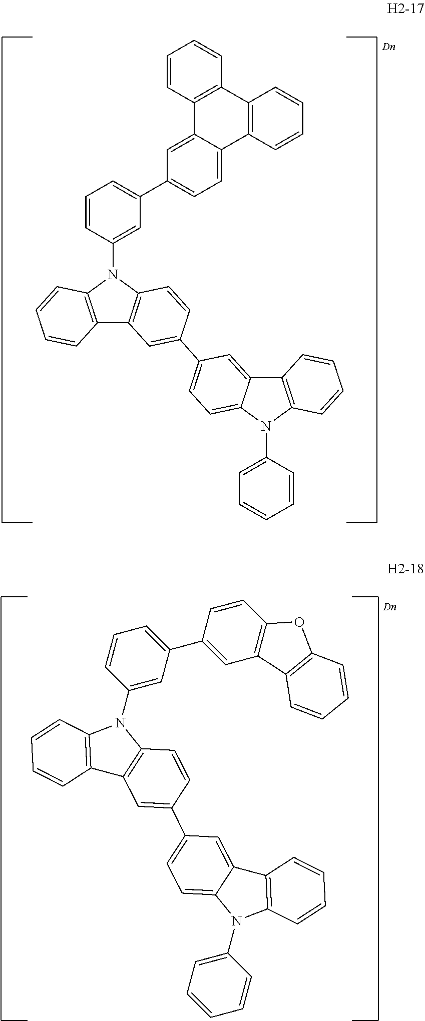 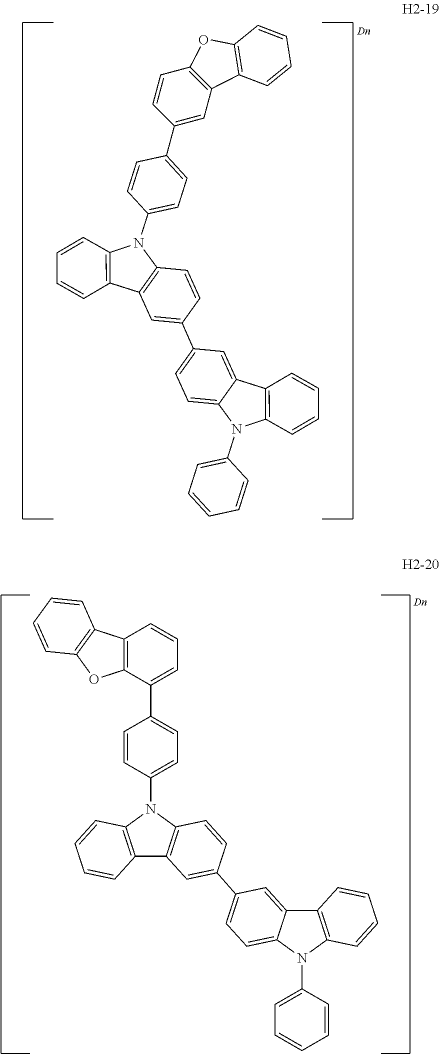 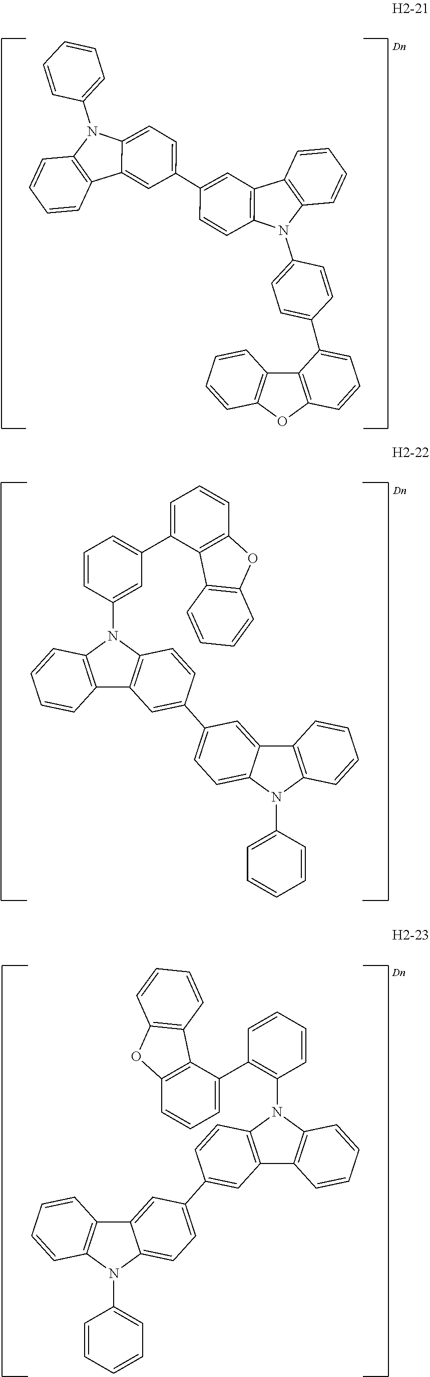 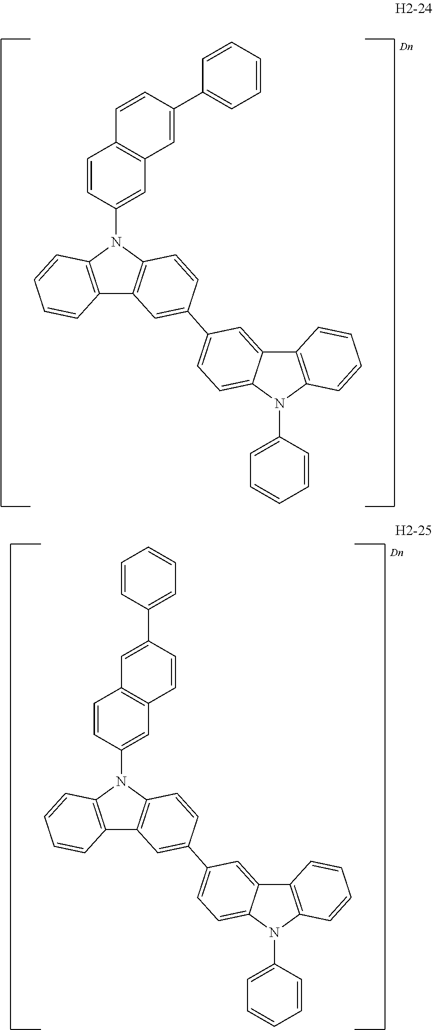 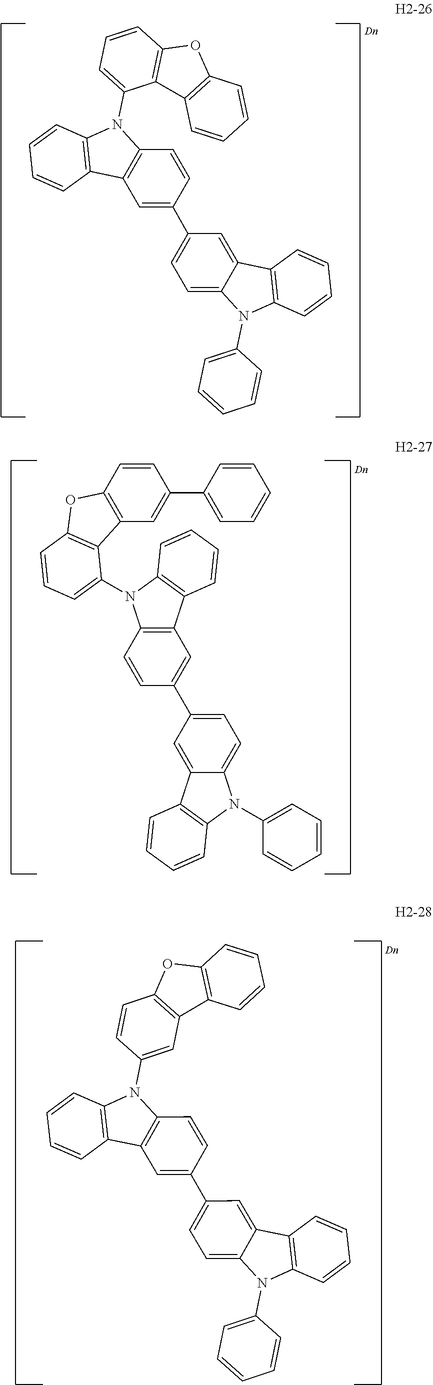 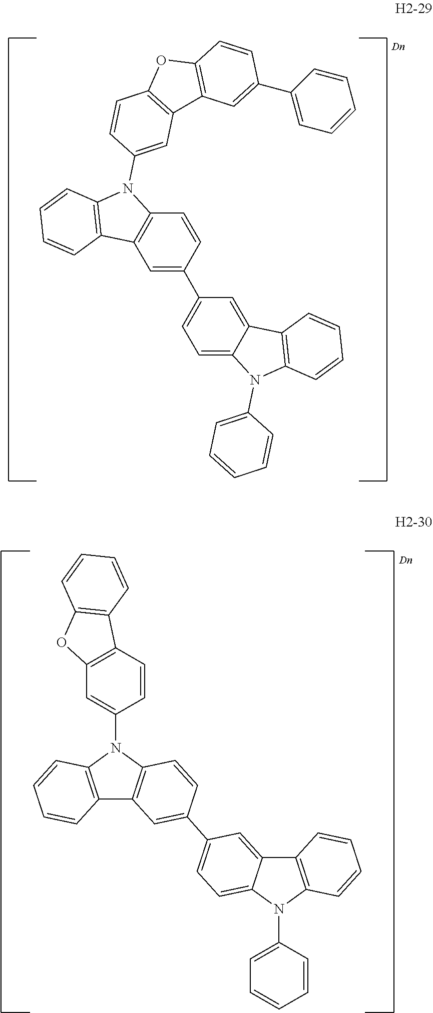 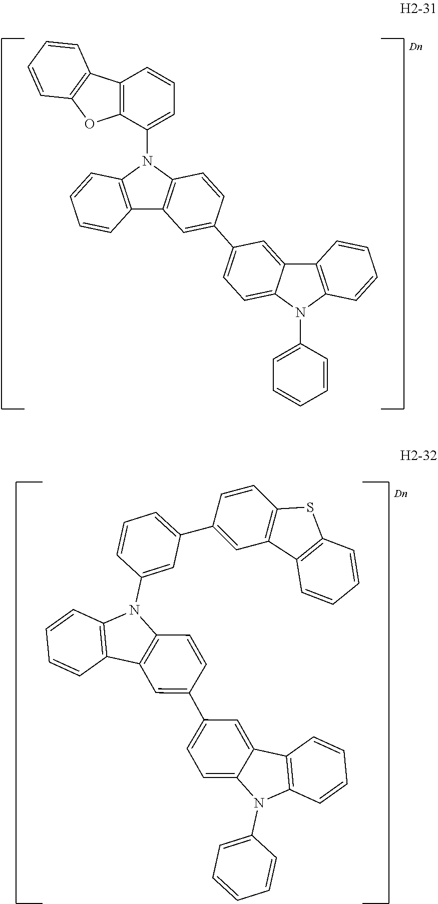 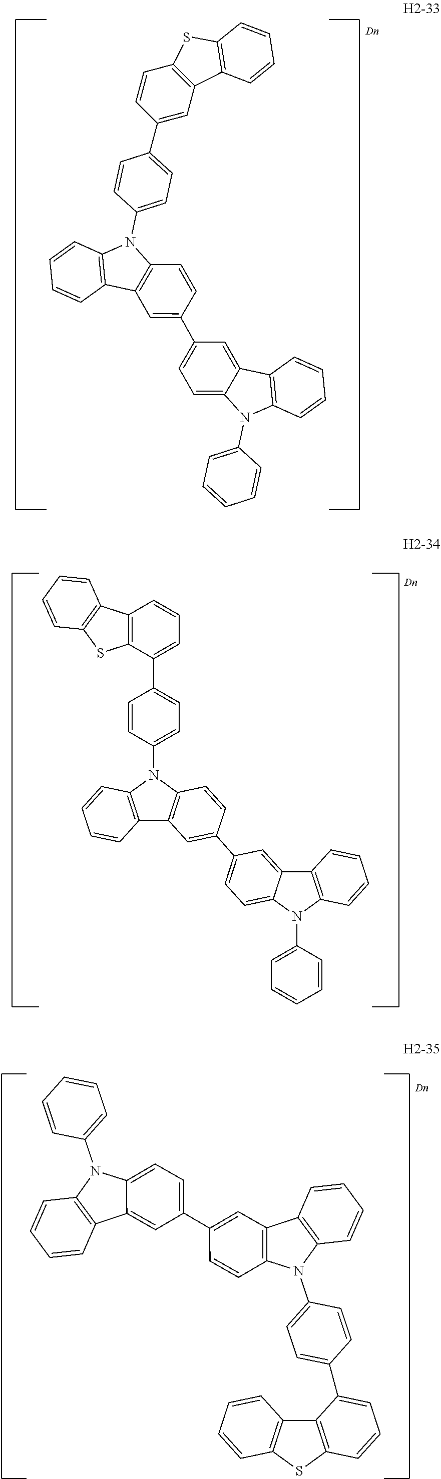 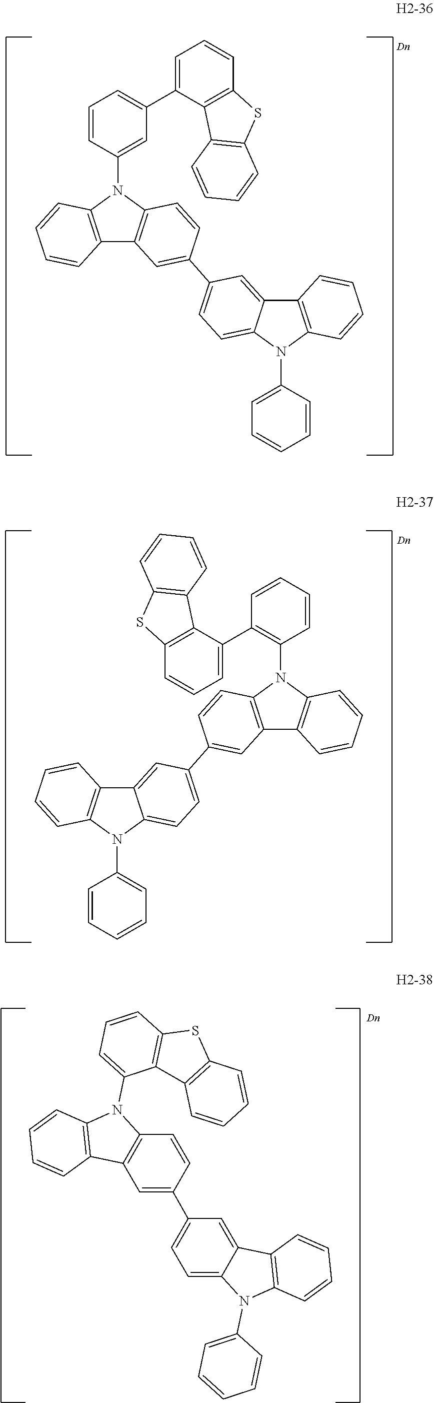 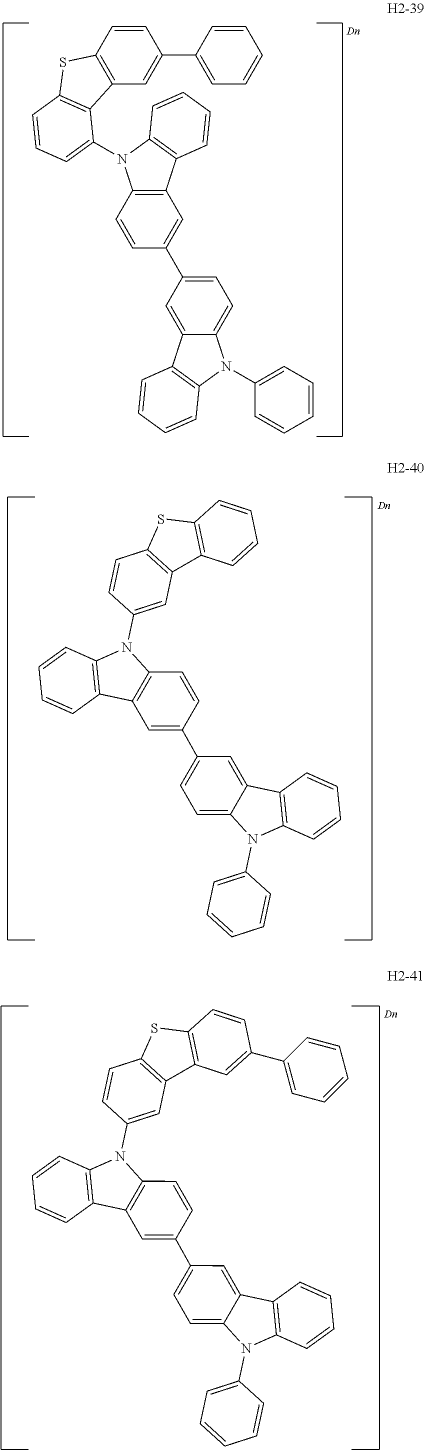 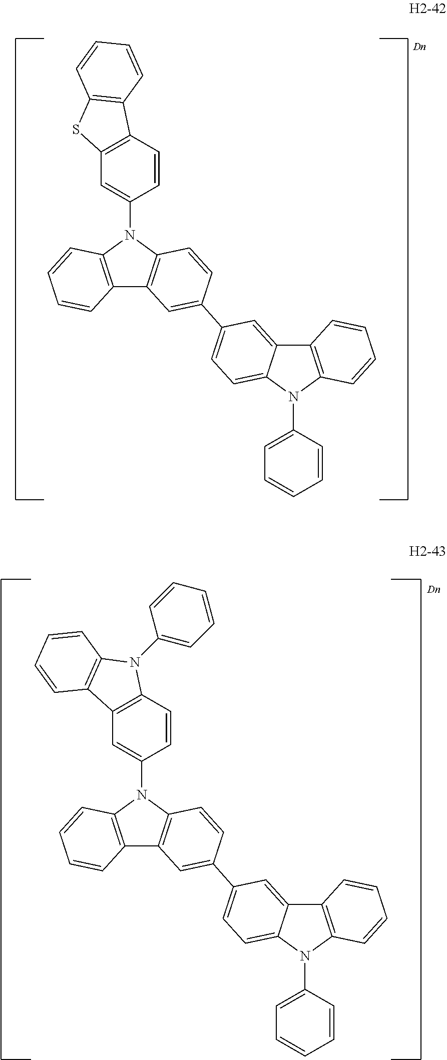 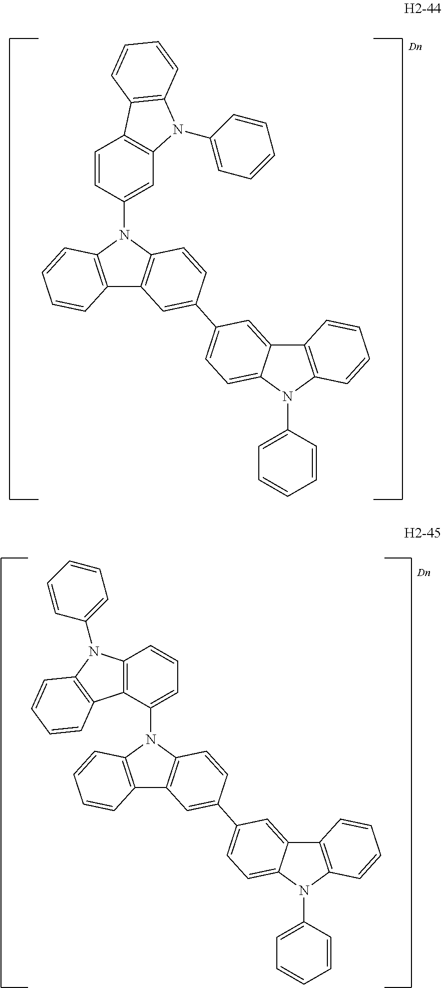 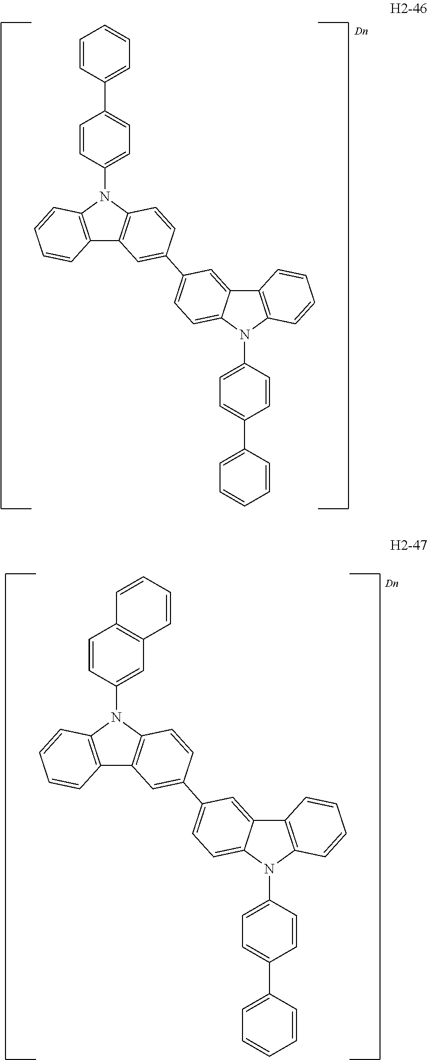 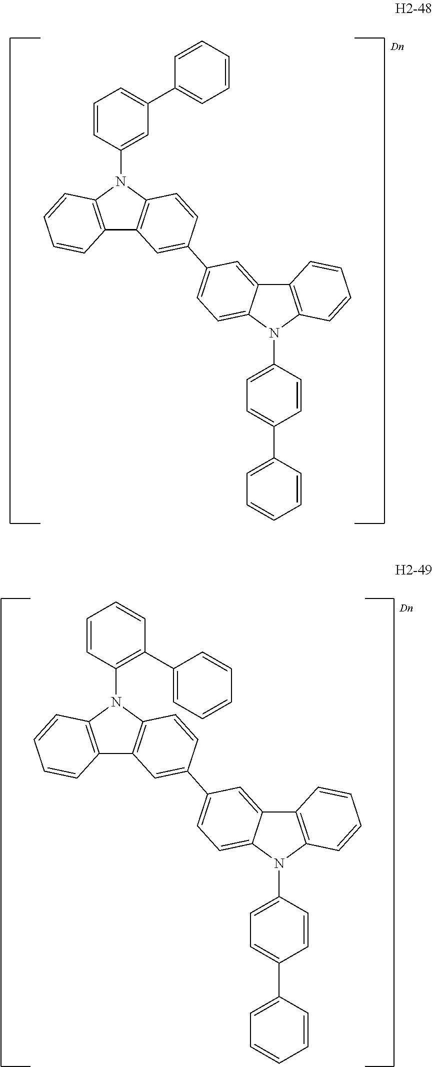 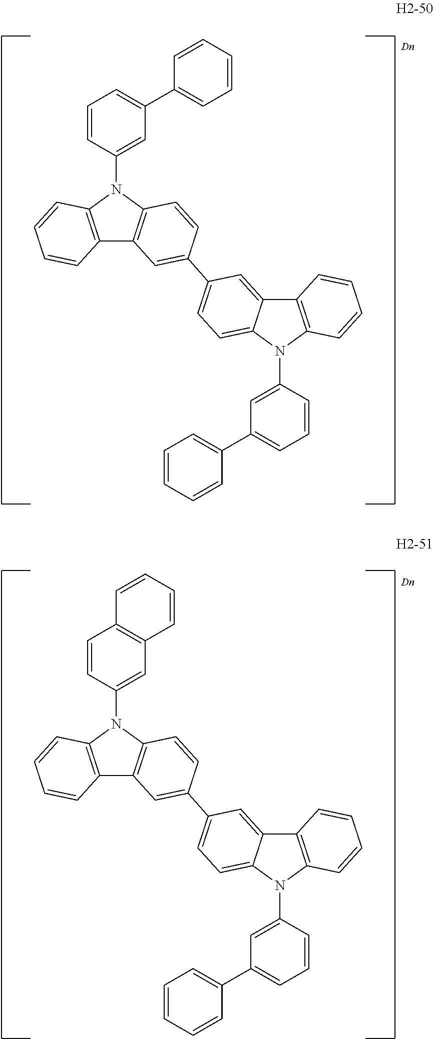 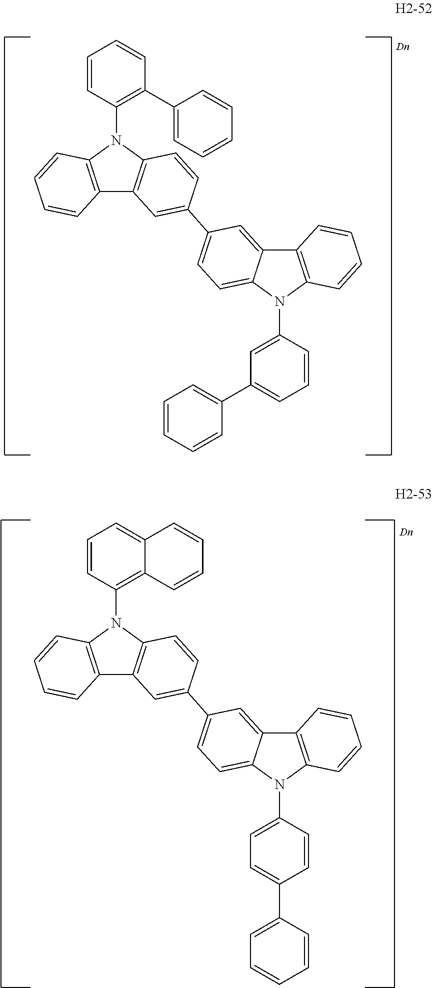 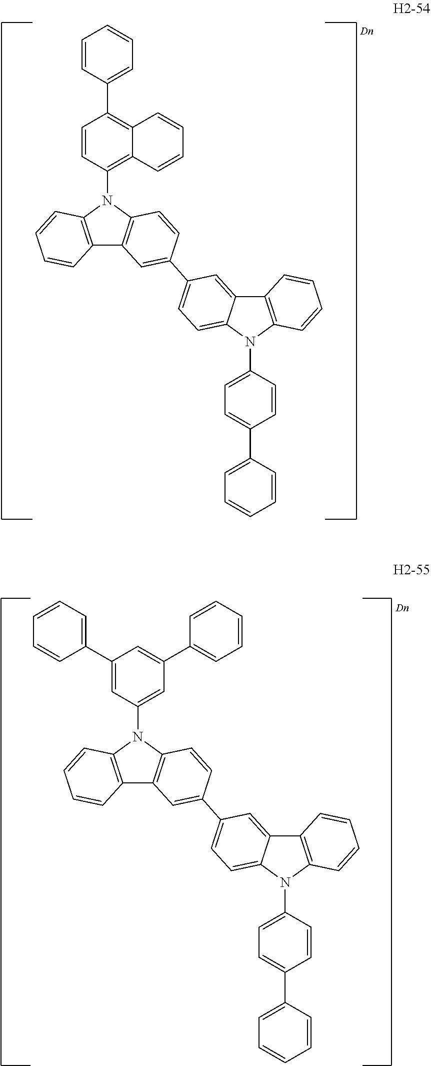 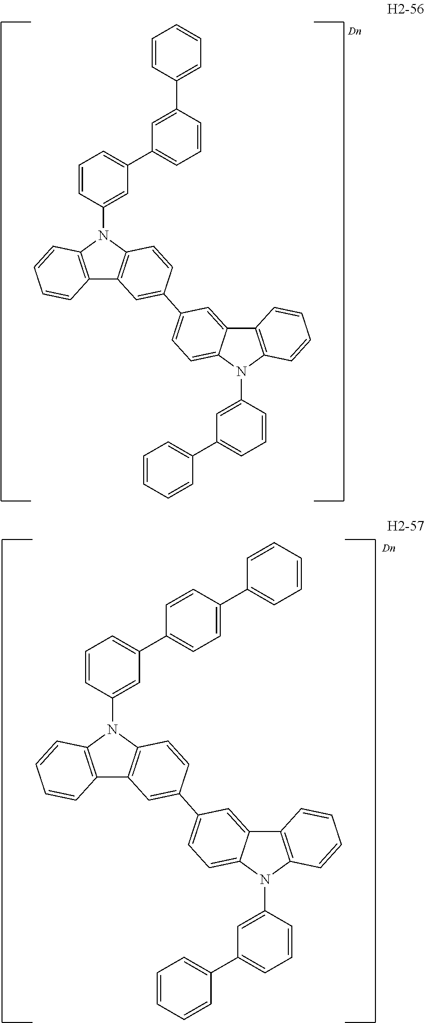 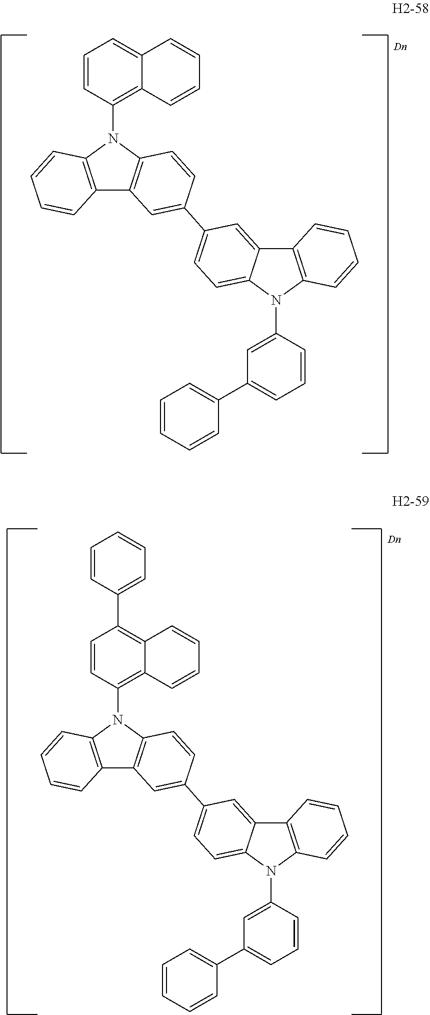 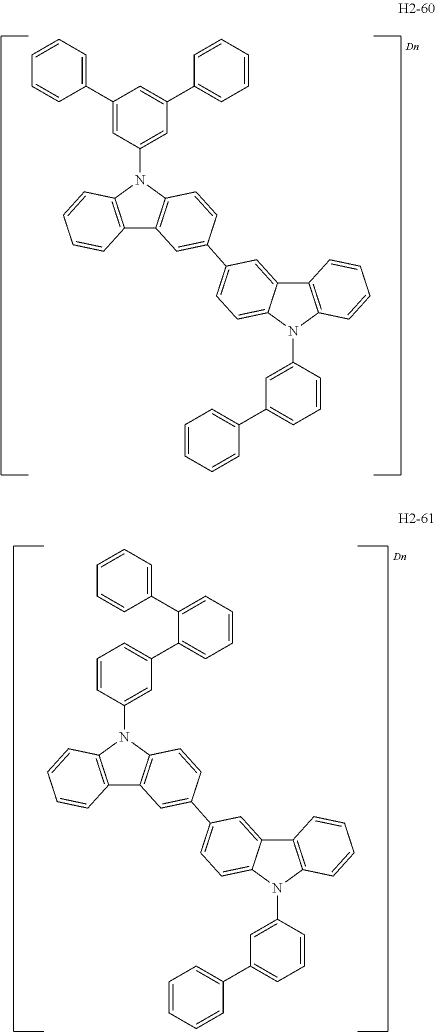 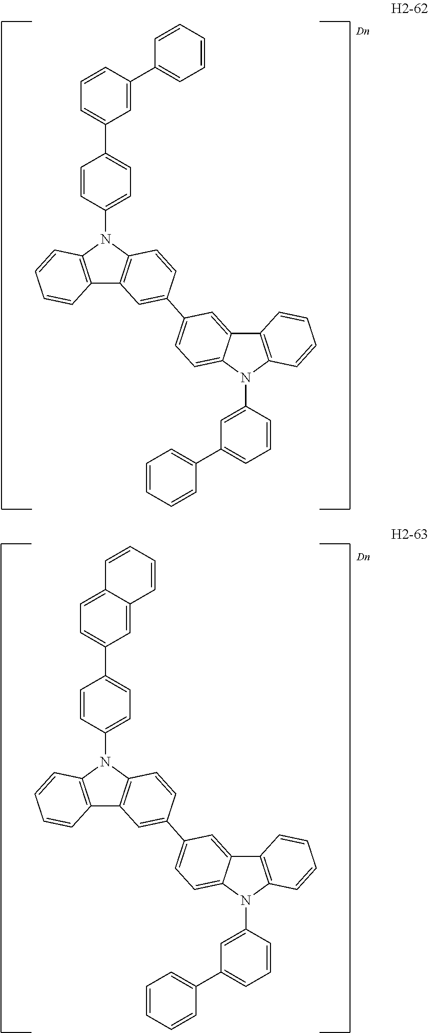 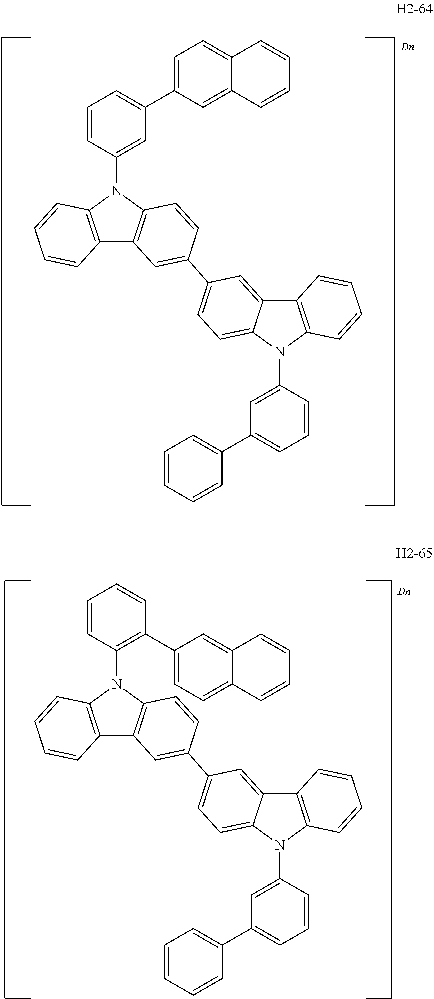 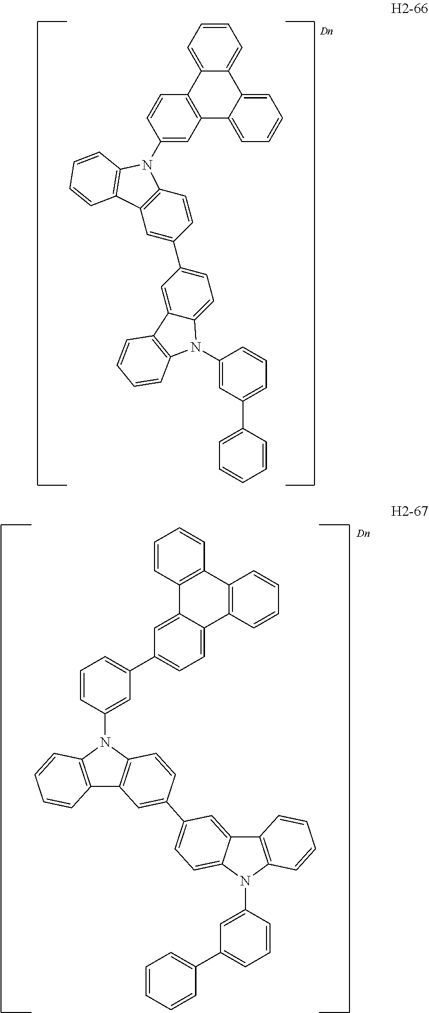 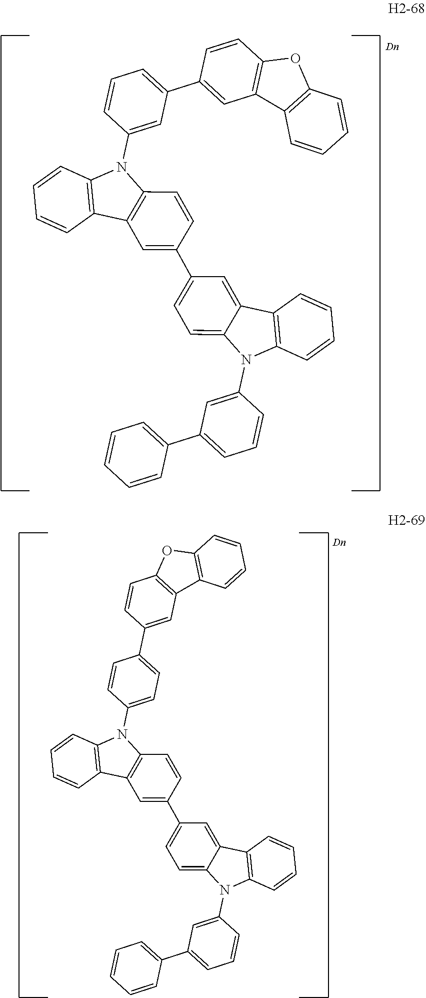 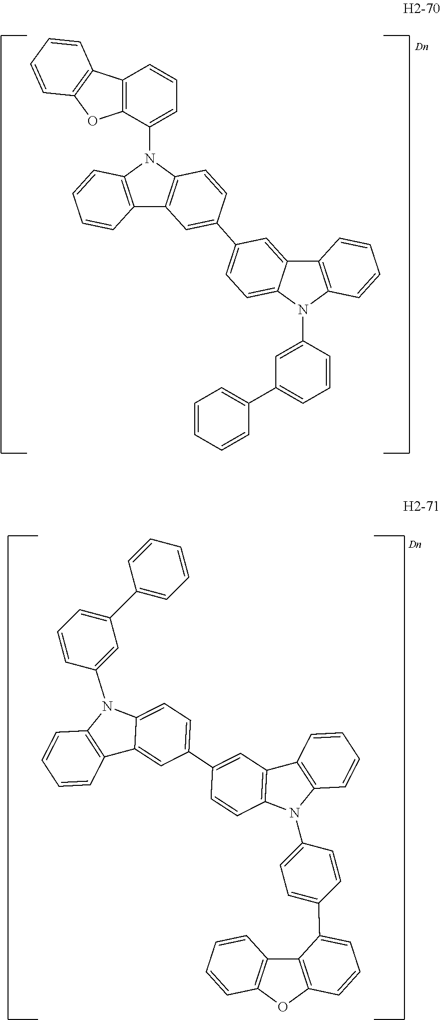 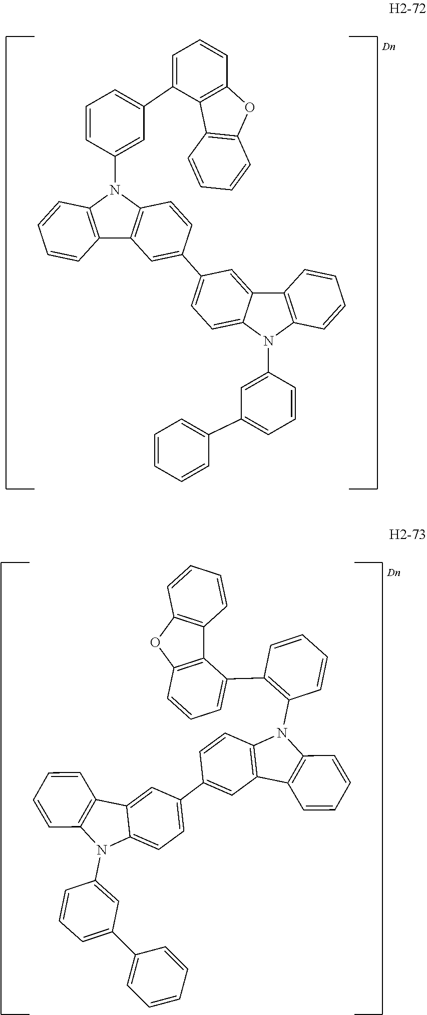 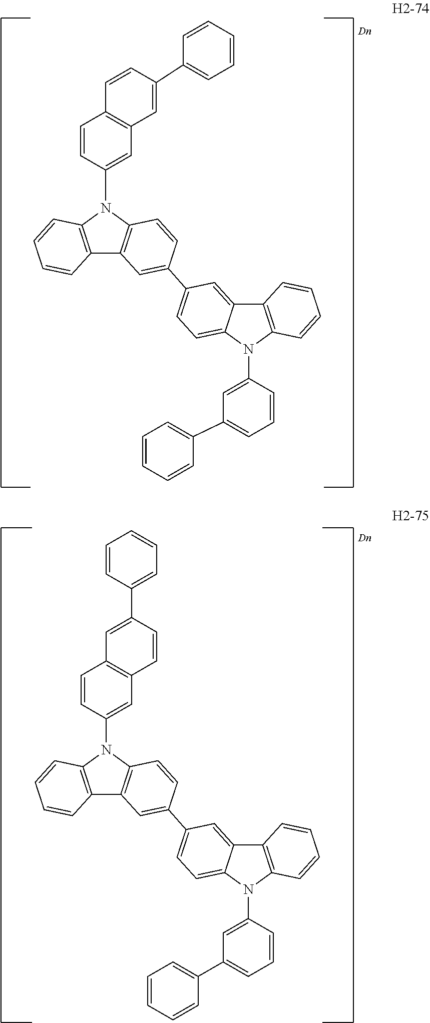 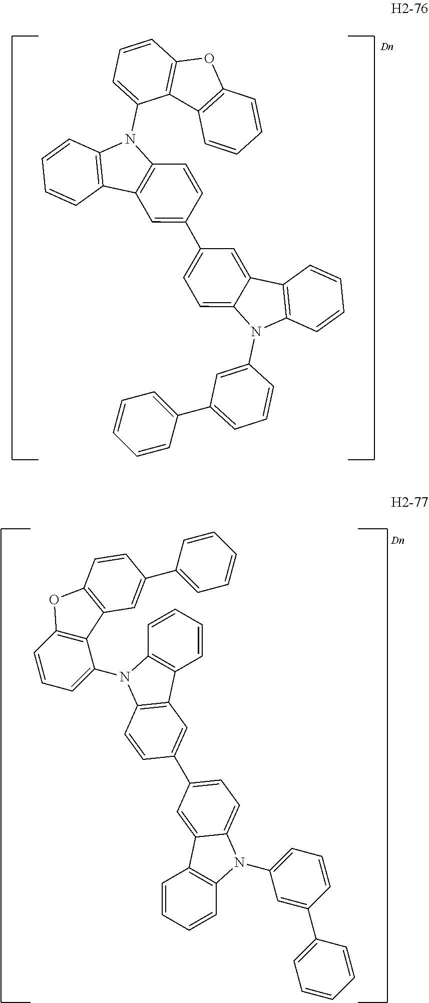 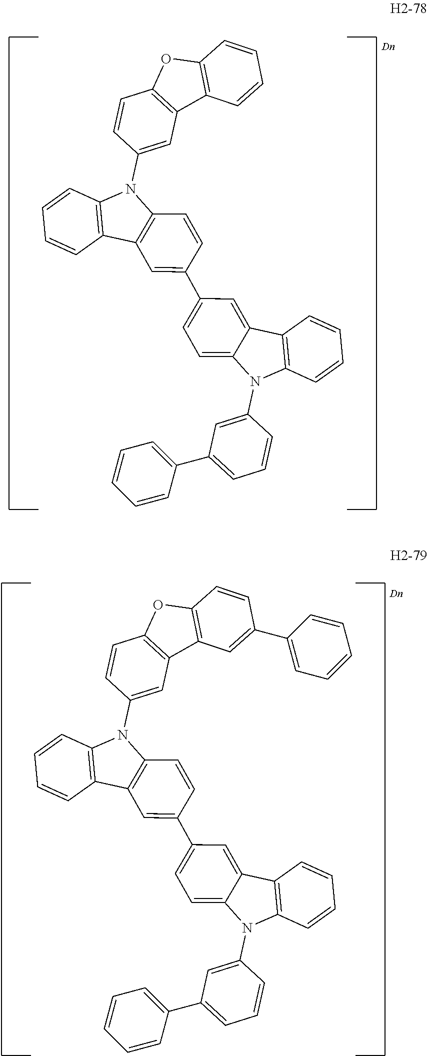 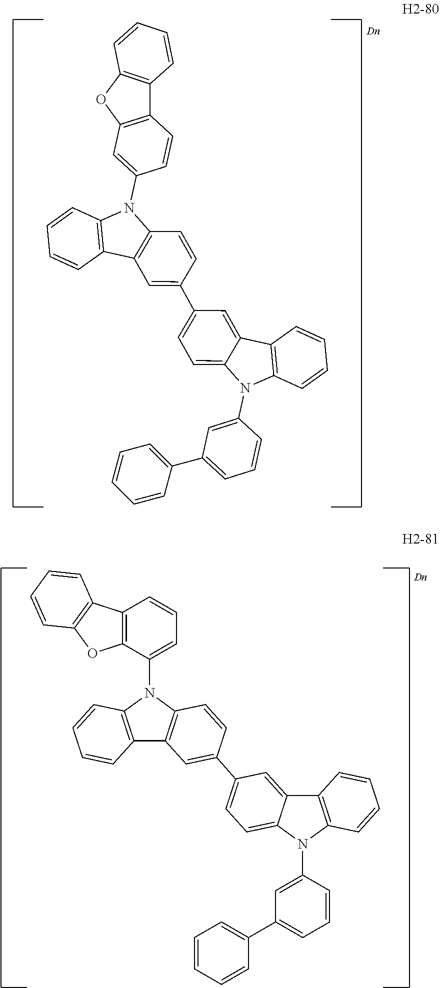 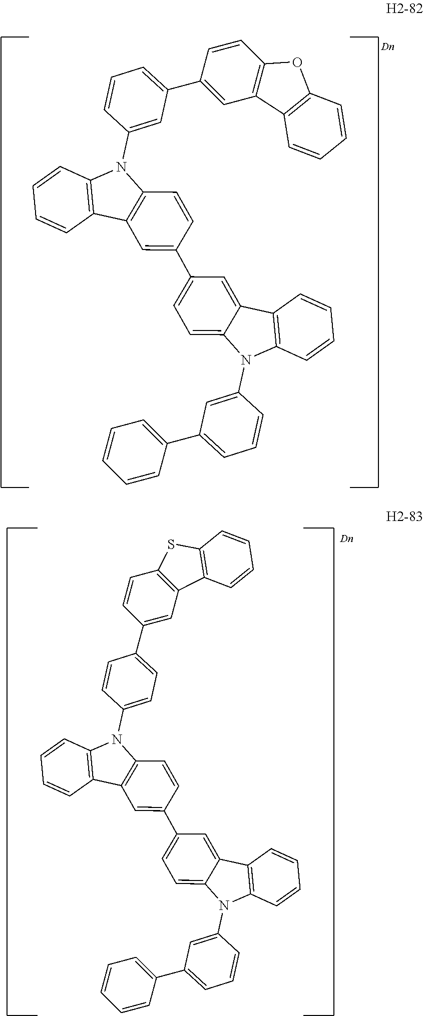 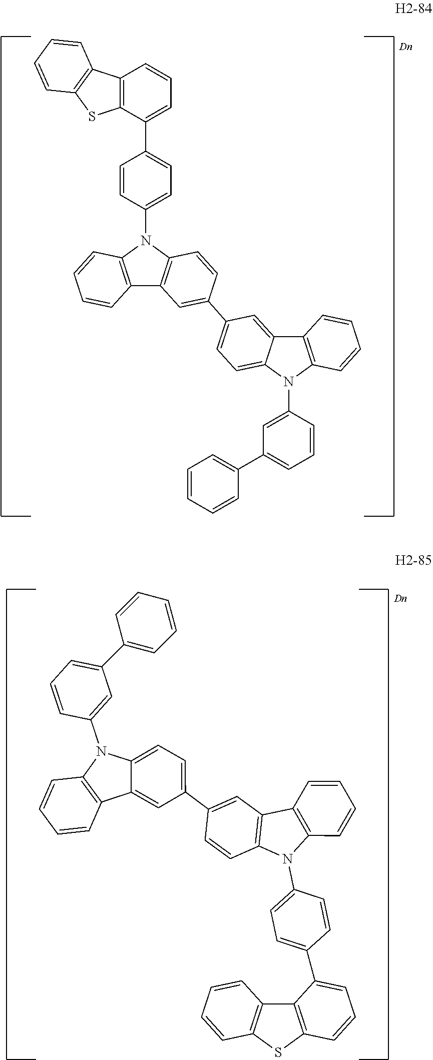 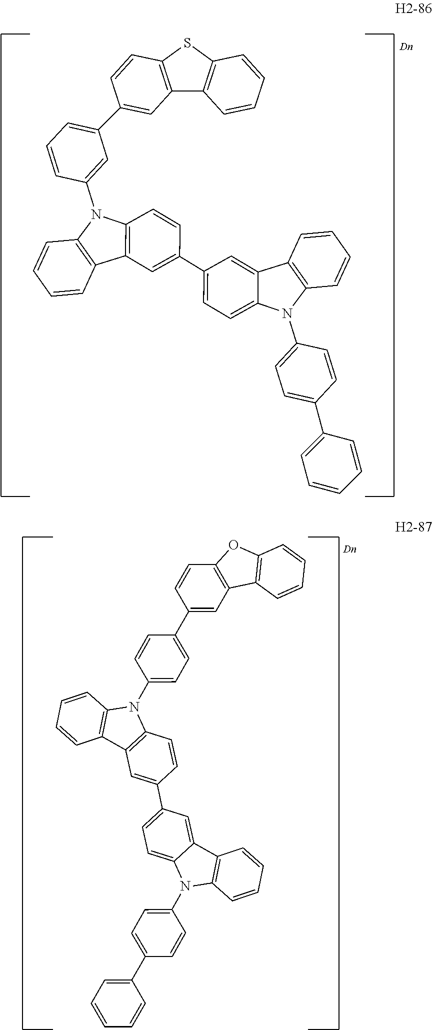 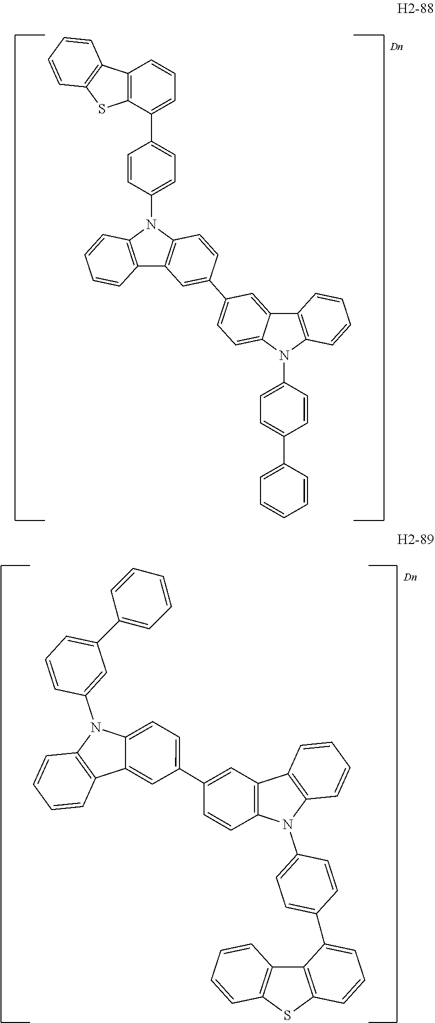 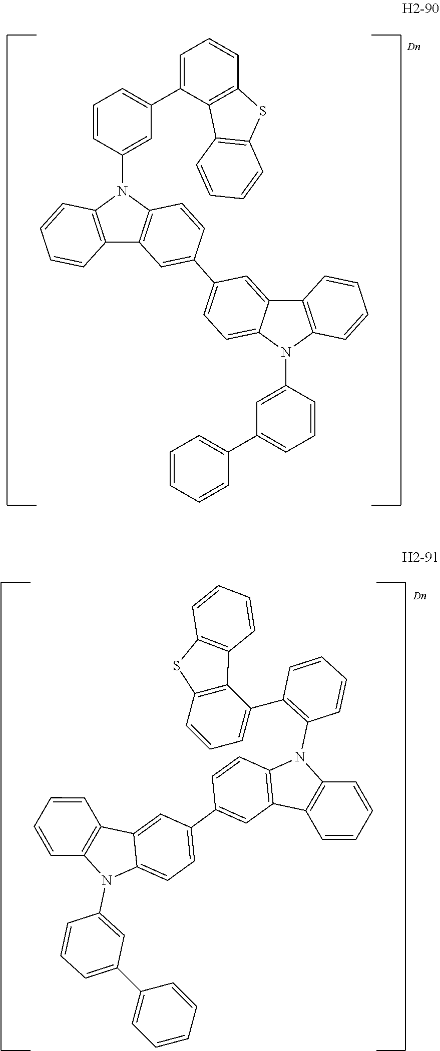 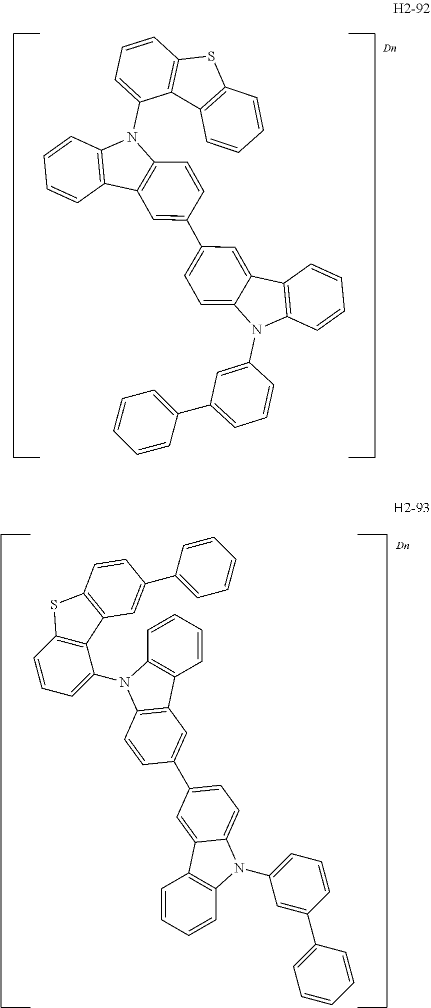 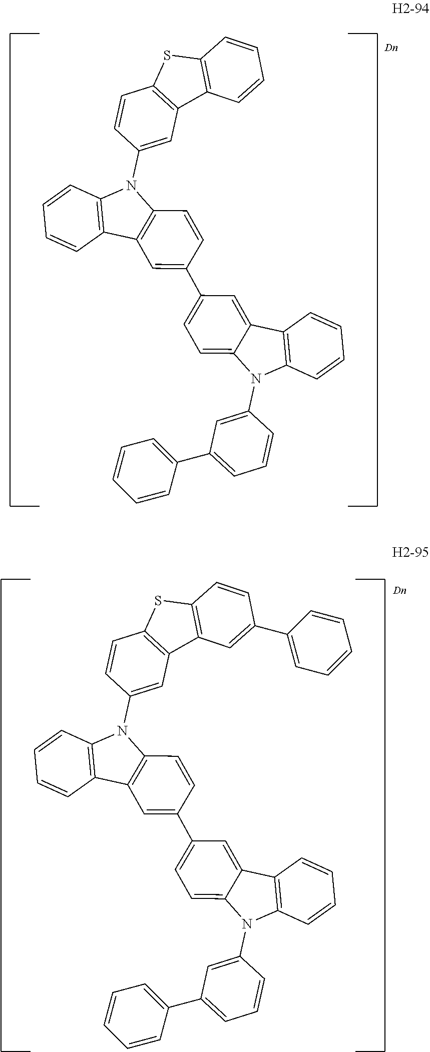 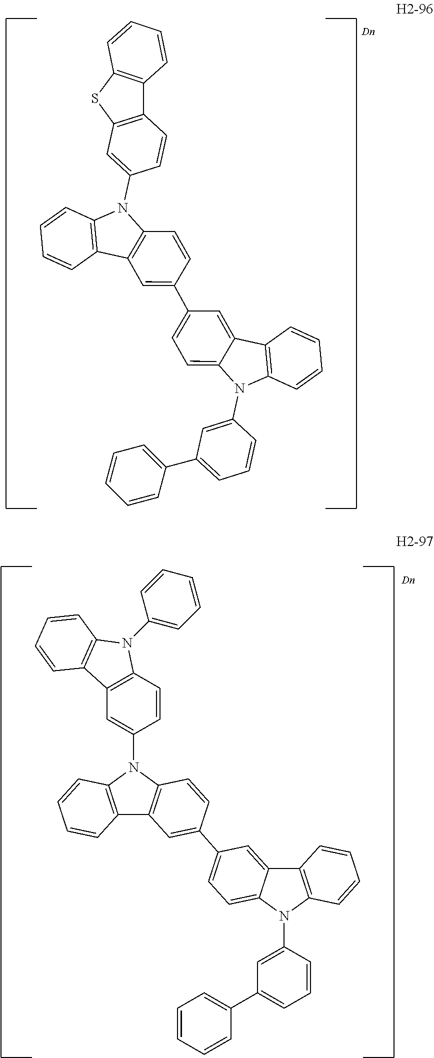 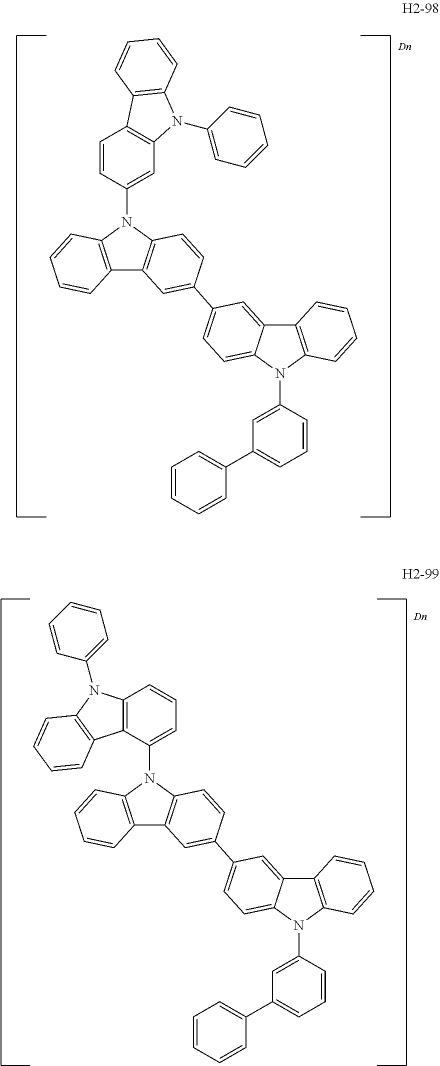 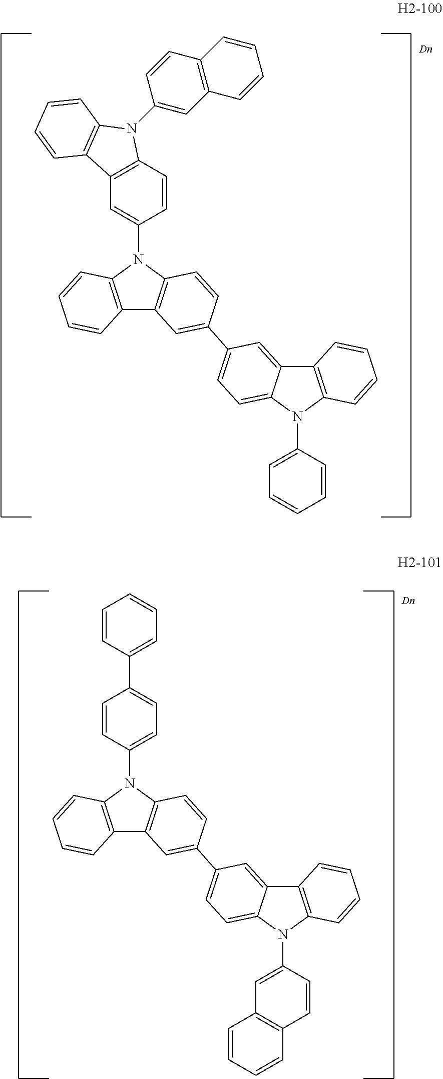 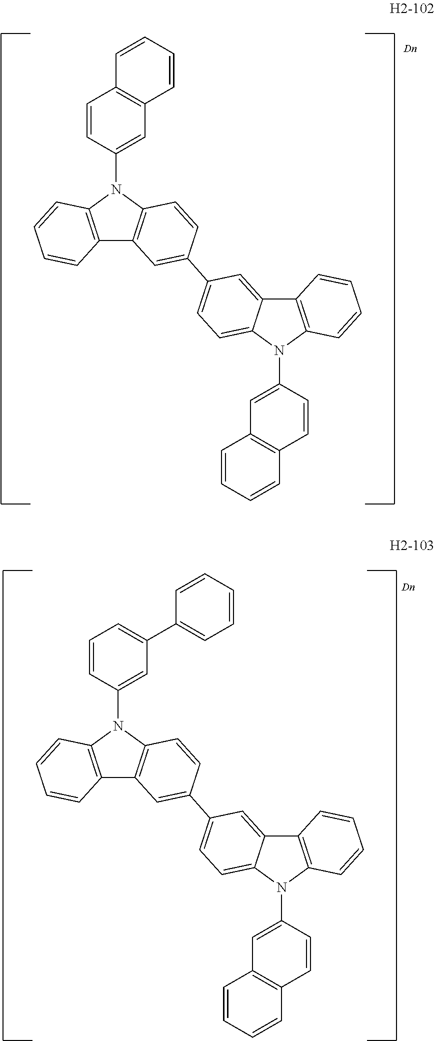 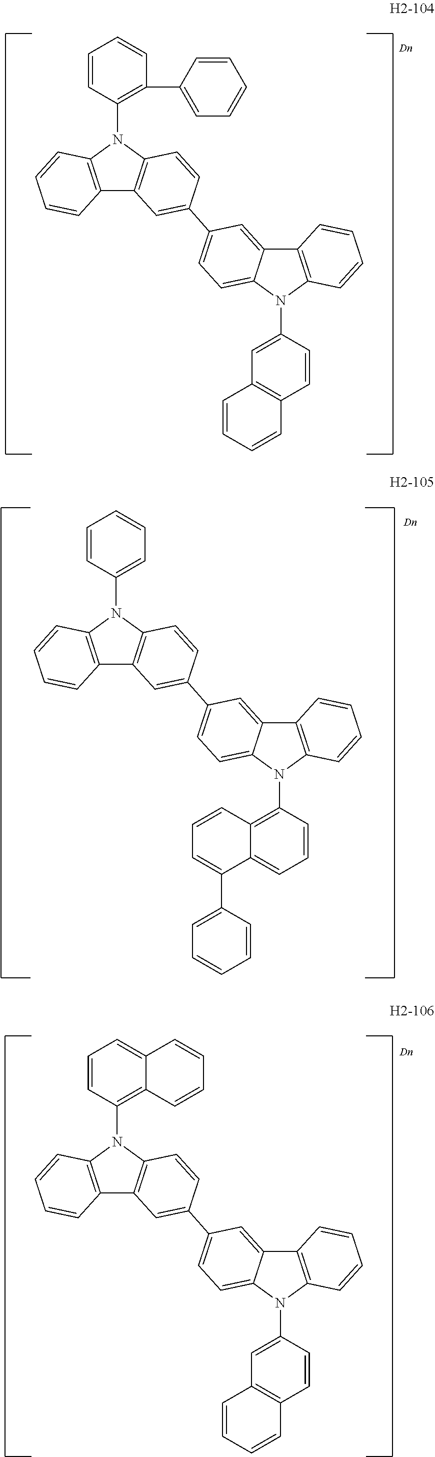 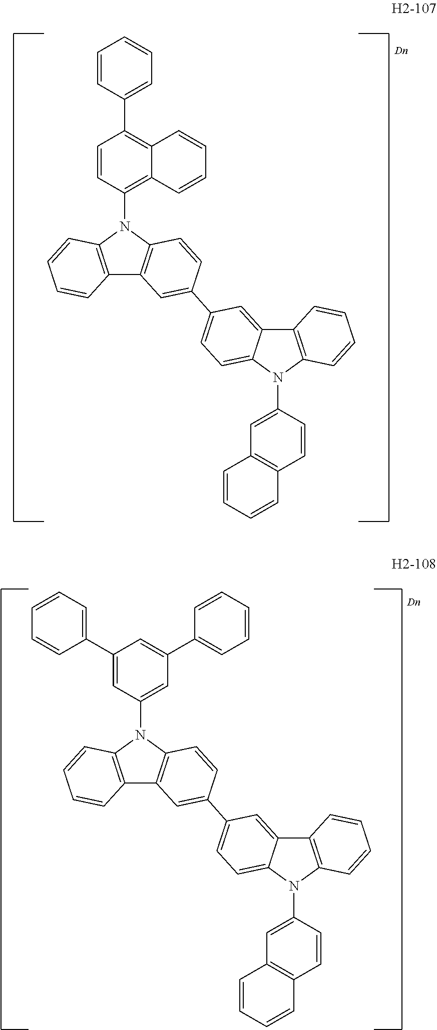 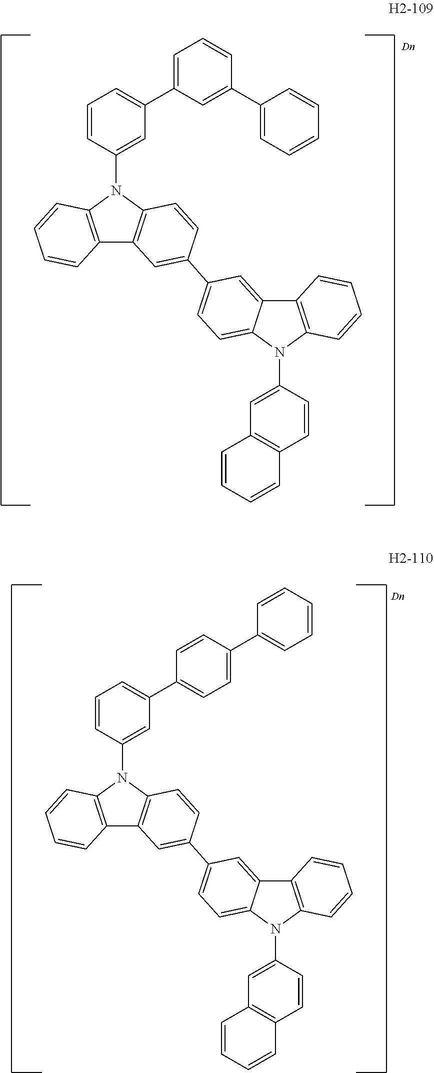 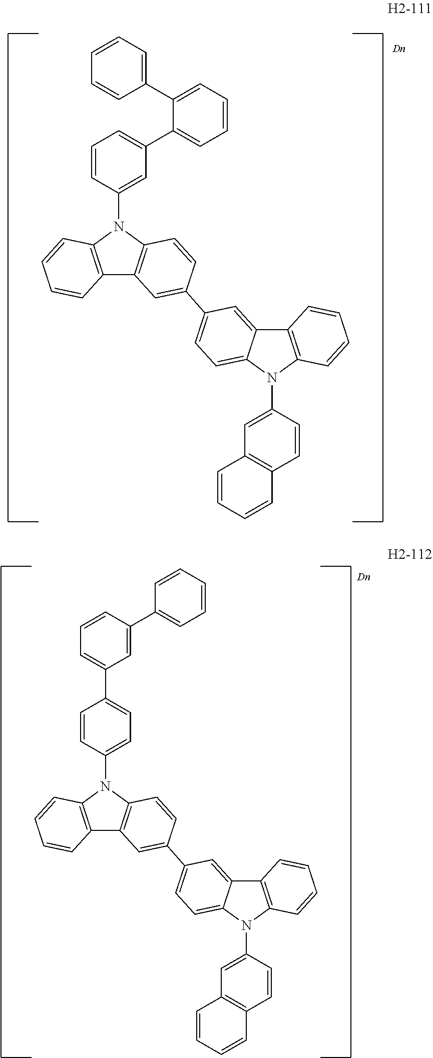 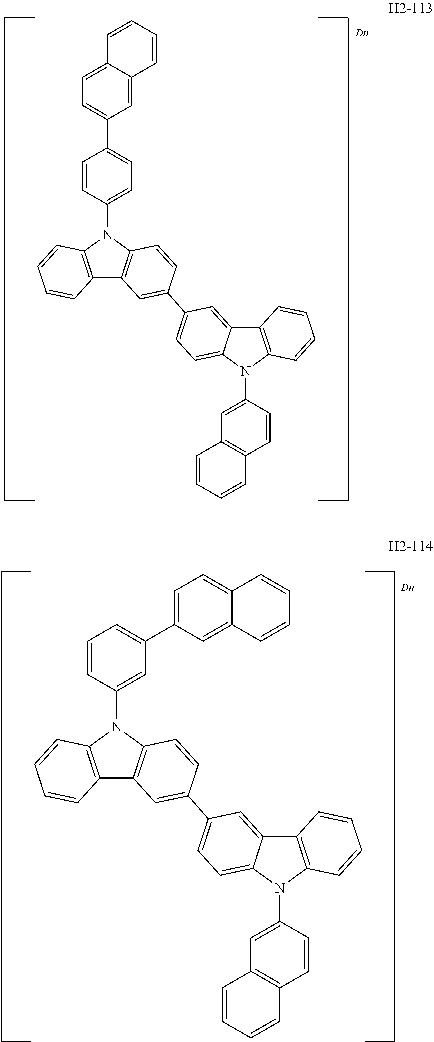 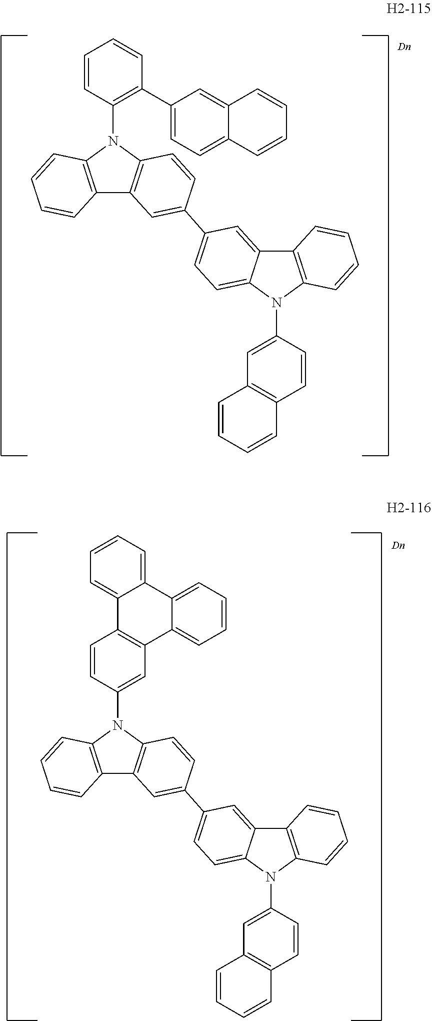 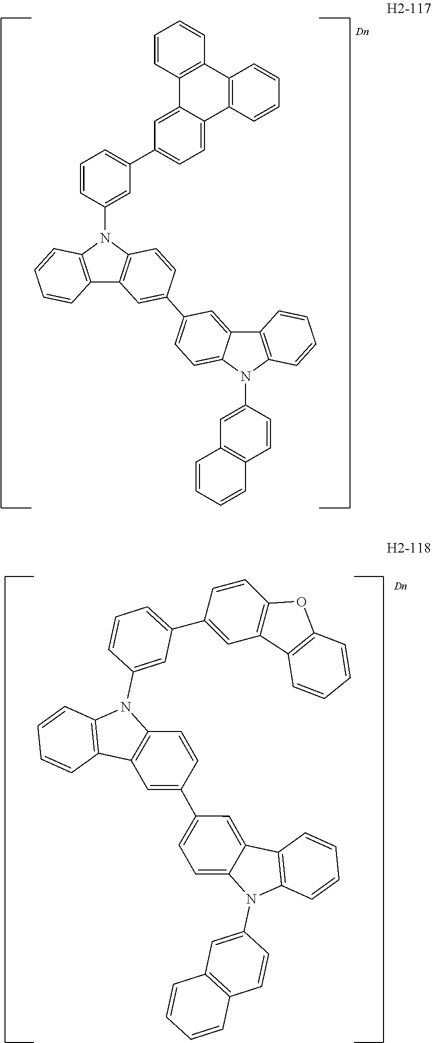 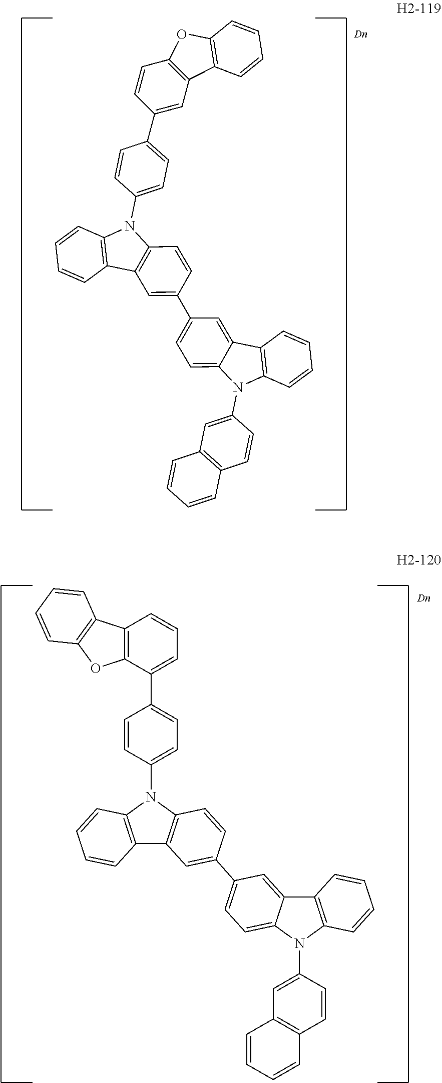 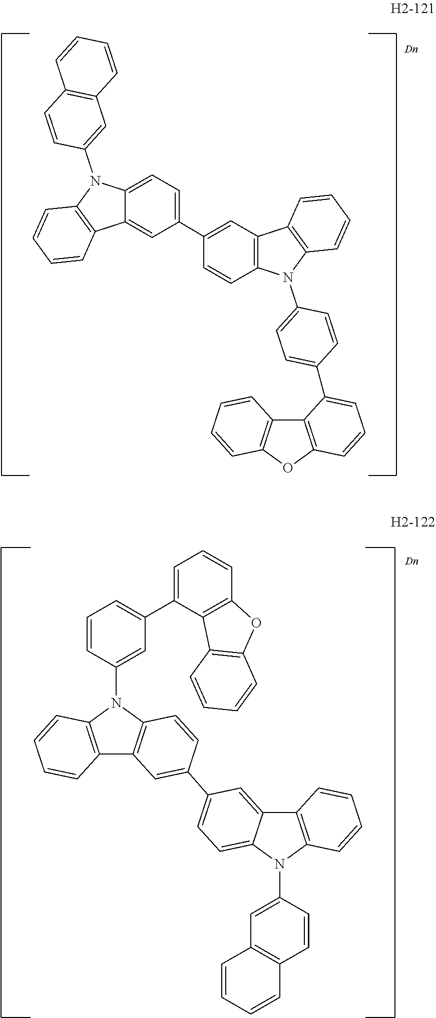 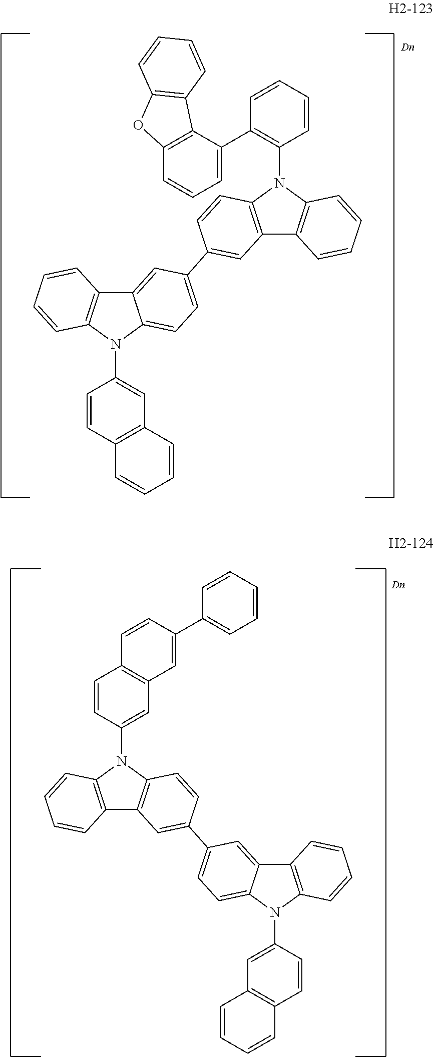 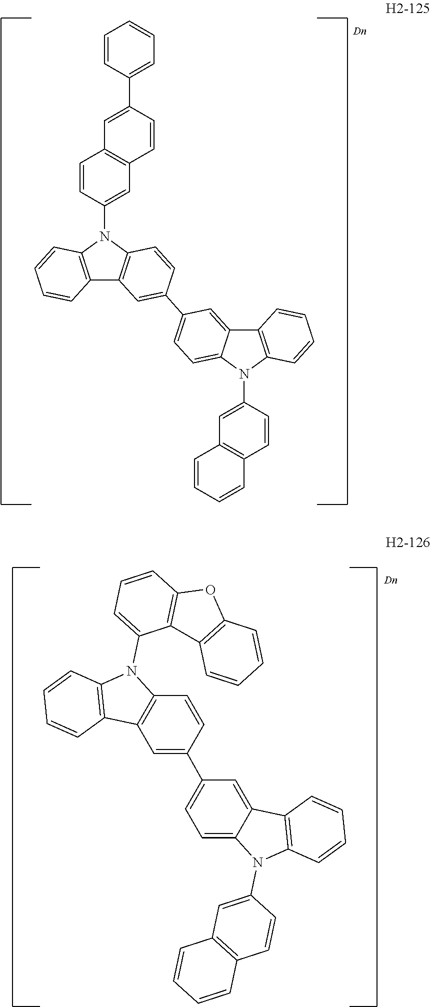 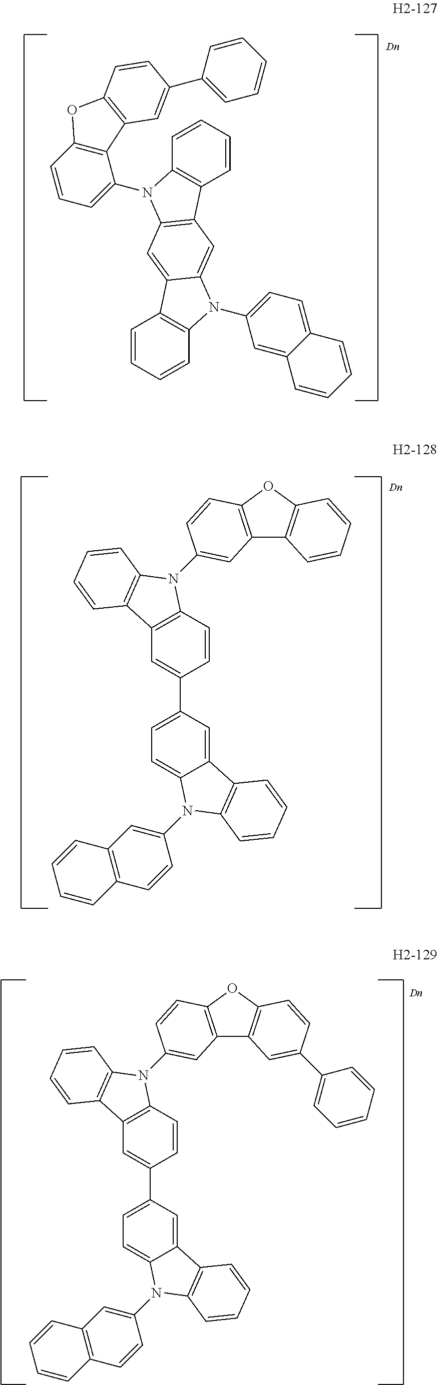 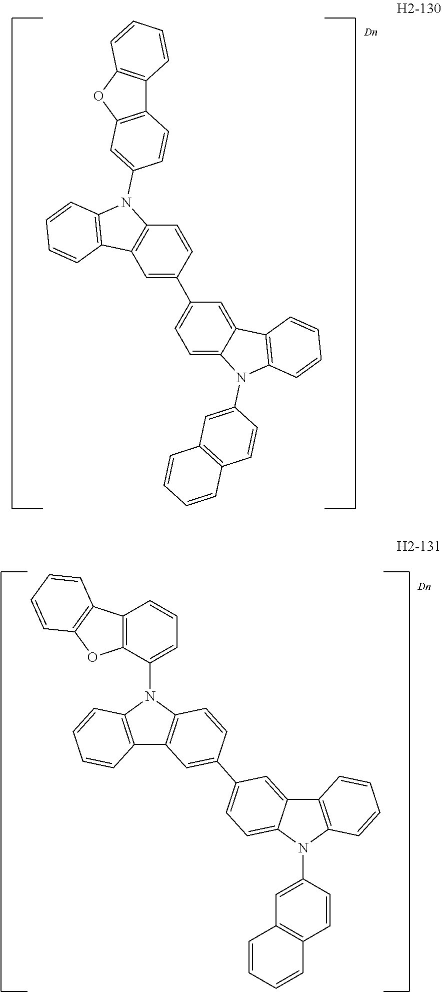 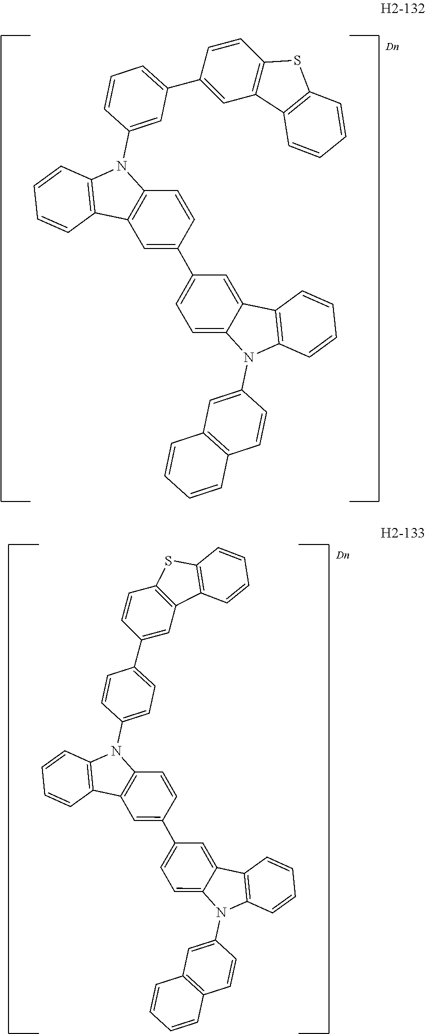 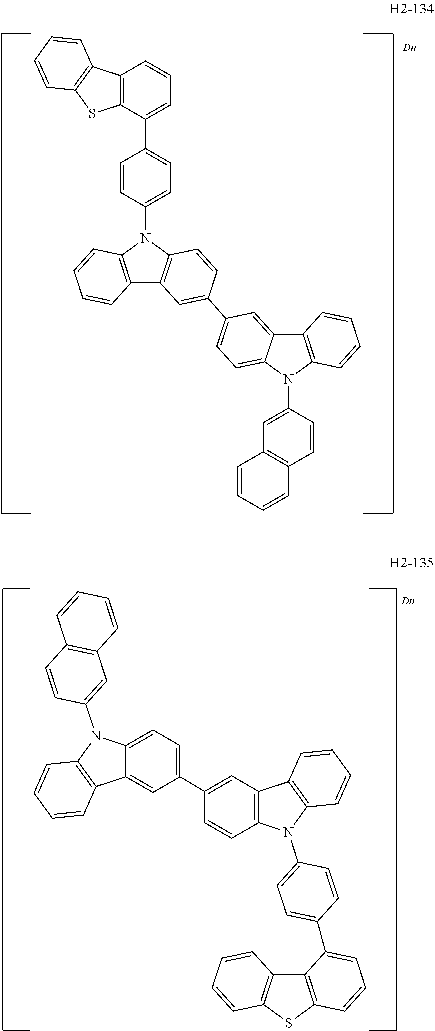 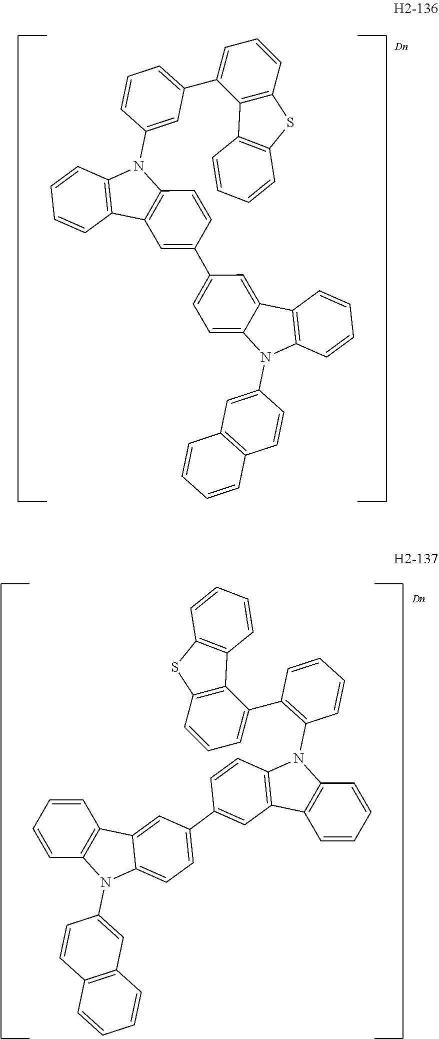 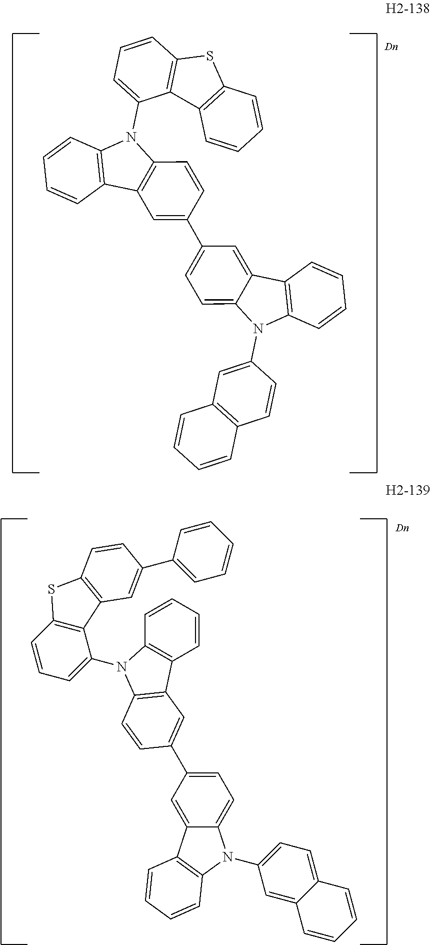 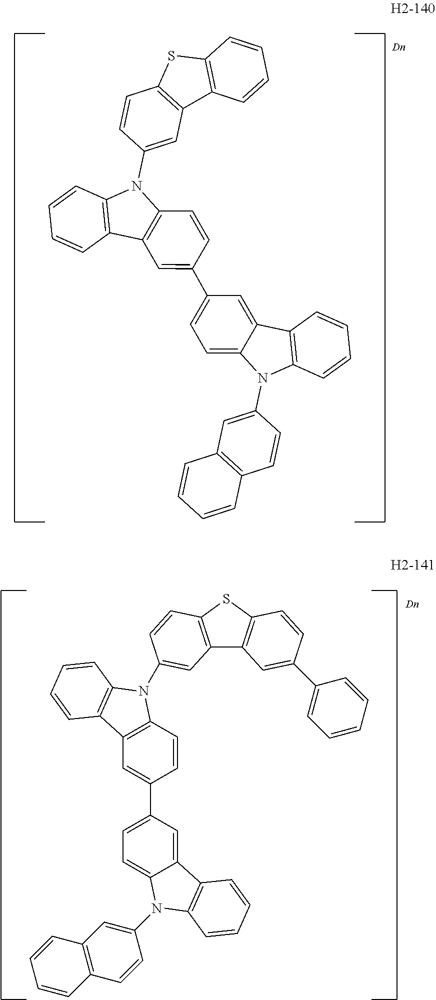 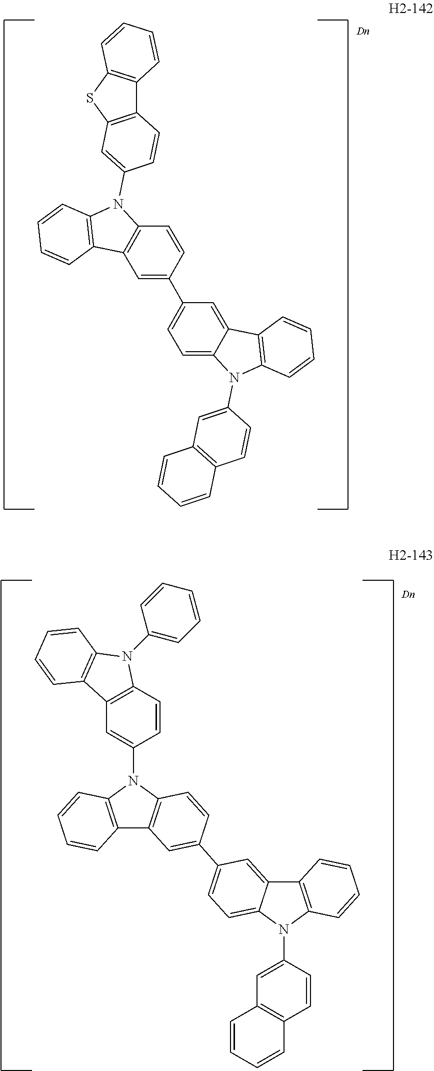 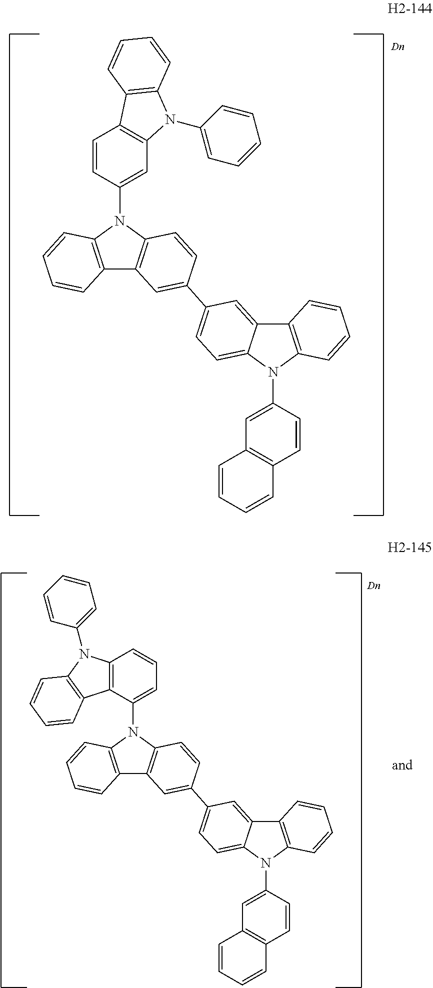 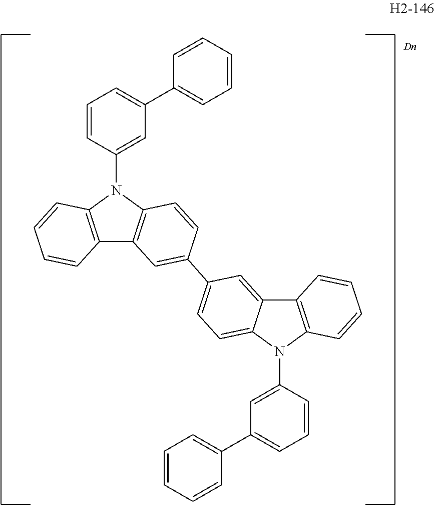
in the compounds above, Dn represents that n number of hydrogens are replaced with deuterium, and n is an integer of 1 to 50.

9. The plurality of host materials according to claim 1, wherein the substituent(s) of the substituted alkyl, the substituted aryl, the substituted arylene, the substituted heteroaryl, the substituted heteroarylene, the substituted dibenzofuranyl, the substituted dibenzothiophenyl, and the substituted carbazolyl, each independently, are at least one selected from the group consisting of deuterium; a halogen; a cyano; a carboxyl; a nitro; a hydroxyl; a (C1-C30)alkyl unsubstituted or substituted with deuterium; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium, a (C1-C30)alkyl(s), a (C6-C30)aryl(s), a (3- to 50-membered)heteroaryl(s), and a di(C6-C30)arylamino(s); a (C6-C30)aryl unsubstituted or substituted with at least one of deuterium, a cyano(s), a (C1-C30)alkyl(s), a (3- to 30-membered)heteroaryl(s), a mono- or di-(C6-C30)arylamino(s), and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

10. An organic electroluminescent device comprising an anode, a cathode, and at least one light-emitting layer between the anode and the cathode, wherein at least one of the light-emitting layers comprises the plurality of host materials according to claim 1.

11. An organic electroluminescent compound represented by the following formula 1-A: 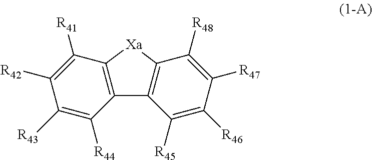
in formula 1-A,
Xₐ represents O or S; and
R₄₁ to R₄₈, each independently, represent hydrogen, deuterium, or a (C6-C18)aryl unsubstituted or substituted with at least one of deuterium, a (C1-C6)alkyl(s) and a (C6-C18)aryl(s), or are represented by the following formula 1-a, with the proviso that at least one of R₄₁ to R₄₈ is represented by the following formula 1-a: 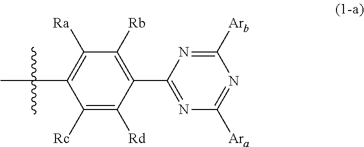
in formula 1-a,
Ra to Rd, each independently, represent hydrogen, deuterium, or a (C6-C18)aryl unsubstituted or substituted with at least one of deuterium, a (C1-C6)alkyl(s) and a (C6-C18)aryl(s); and
Arₐ and Ar_{b}, each independently, represent a phenyl unsubstituted or substituted with deuterium, a naphthyl unsubstituted or substituted with deuterium, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Arₐ and Arₜ comprises a substituted or unsubstituted carbazolyl.

12. The organic electroluminescent compound according to claim 11, wherein at least one of Arₐ and Ar_{b} in formula 1-a is represented by the following formula 1-b1 or 1-b2: 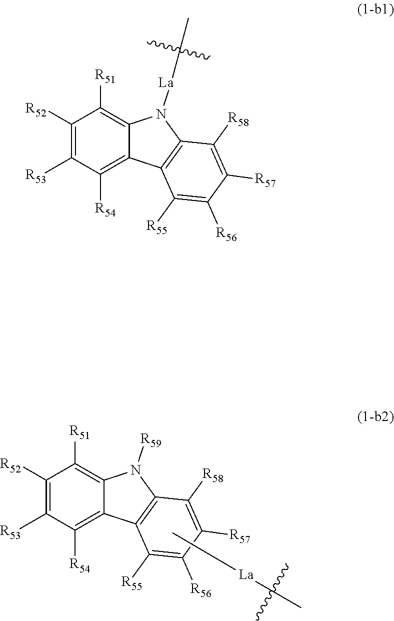
in formulas 1-bi and 1-b2,
La represents a single bond, a phenylene unsubstituted or substituted with deuterium, a naphthylene unsubstituted or substituted with deuterium, a biphenylene unsubstituted or substituted with deuterium, or a terphenylene unsubstituted or substituted with deuterium; and
R₅₁ to R₅₉, each independently, represent hydrogen, deuterium, a phenyl unsubstituted or substituted with deuterium, a naphthyl unsubstituted or substituted with deuterium, a biphenyl unsubstituted or substituted with deuterium, a terphenyl unsubstituted or substituted with deuterium, or a combination thereof.

13. The organic electroluminescent compound according to claim 11, wherein the compound represented by formula 1-A is selected from the group consisting of the following compounds:

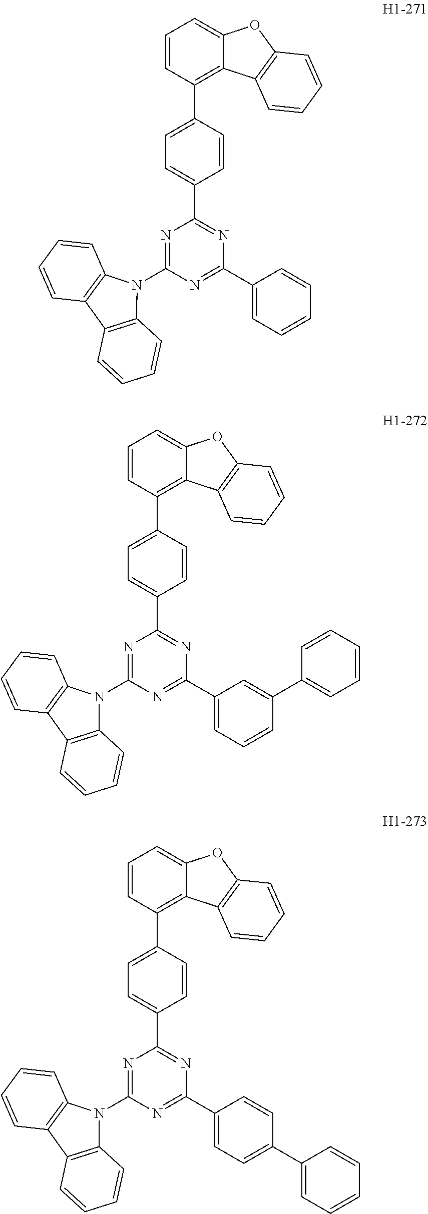 [SMILES: c1ccc(-c2nc(-c3ccc(-c4cccc5oc6ccccc6c45)cc3)nc(-n3c4ccccc4c4ccccc43)n2)cc1 ; c1ccc(-c2cccc(-c3nc(-c4ccc(-c5cccc6oc7ccccc7c56)cc4)nc(-n4c5ccccc5c5ccccc54)n3)c2)cc1 ; c1ccc(-c2ccc(-c3nc(-c4ccc(-c5cccc6oc7ccccc7c56)cc4)nc(-n4c5ccccc5c5ccccc54)n3)cc2)cc1]

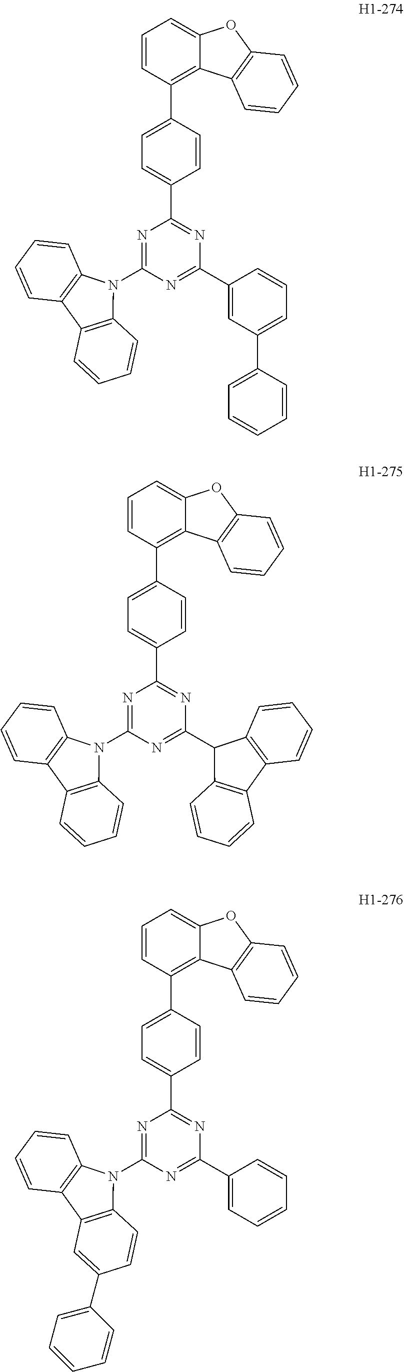 [SMILES: c1ccc(-c2cccc(-c3nc(-c4ccc(-c5cccc6oc7ccccc7c56)cc4)nc(-n4c5ccccc5c5ccccc54)n3)c2)cc1 ; c1ccc2c(c1)-c1ccccc1C2c1nc(-c2ccc(-c3cccc4oc5ccccc5c34)cc2)nc(-n2c3ccccc3c3ccccc32)n1 ; c1ccc(-c2ccc3c(c2)c2ccccc2n3-c2nc(-c3ccccc3)nc(-c3ccc(-c4cccc5oc6ccccc6c45)cc3)n2)cc1]

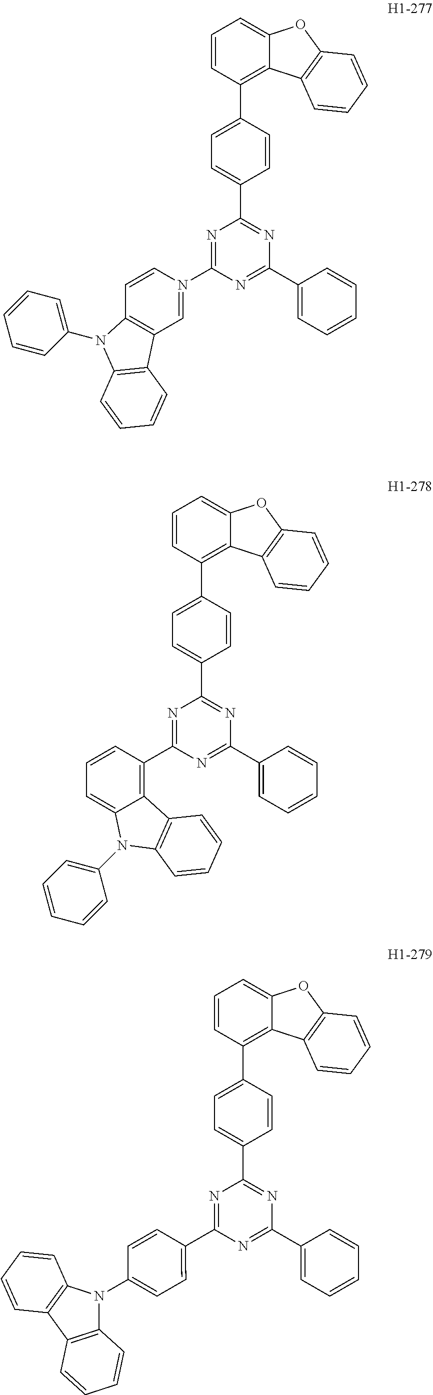

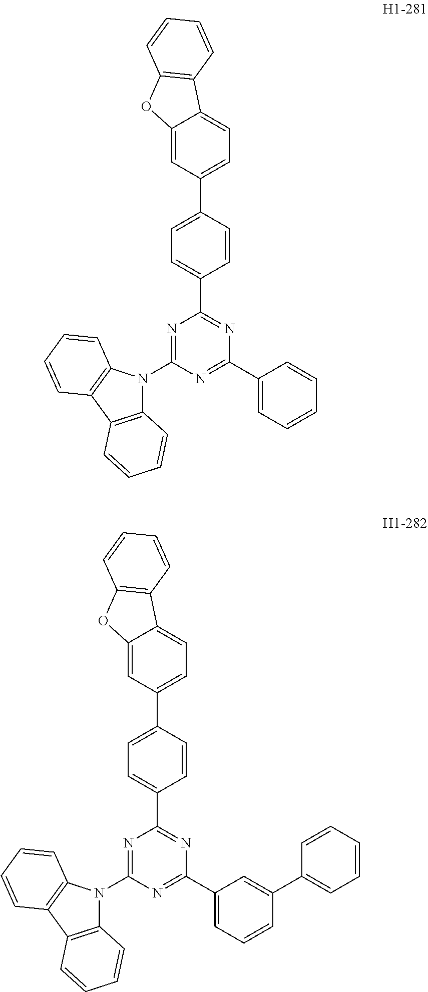 [SMILES: c1ccc(-c2nc(-c3ccc(-c4ccc5c(c4)oc4ccccc45)cc3)nc(-n3c4ccccc4c4ccccc43)n2)cc1 ; c1ccc(-c2cccc(-c3nc(-c4ccc(-c5ccc6c(c5)oc5ccccc56)cc4)nc(-n4c5ccccc5c5ccccc54)n3)c2)cc1]

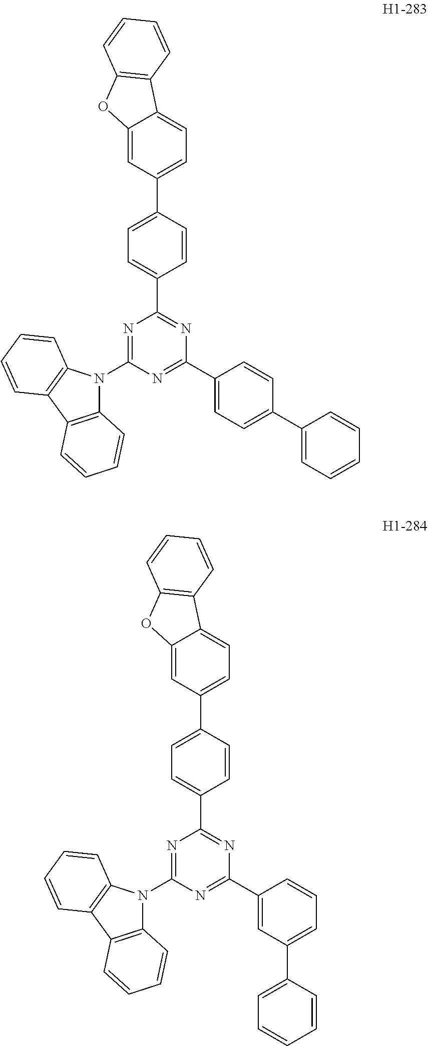 [SMILES: c1ccc(-c2ccc(-c3nc(-c4ccc(-c5ccc6c(c5)oc5ccccc56)cc4)nc(-n4c5ccccc5c5ccccc54)n3)cc2)cc1 ; c1ccc(-c2cccc(-c3nc(-c4ccc(-c5ccc6c(c5)oc5ccccc56)cc4)nc(-n4c5ccccc5c5ccccc54)n3)c2)cc1]

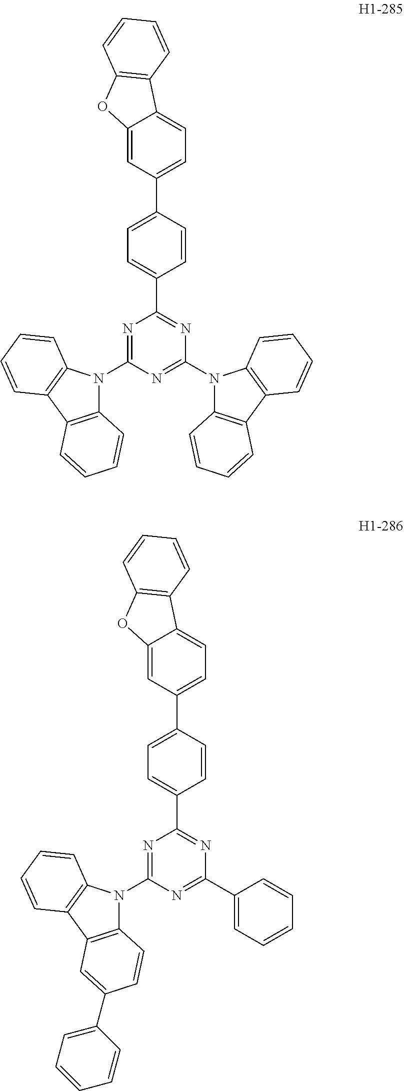 [SMILES: c1ccc2c(c1)oc1cc(-c3ccc(-c4nc(-n5c6ccccc6c6ccccc65)nc(-n5c6ccccc6c6ccccc65)n4)cc3)ccc12 ; c1ccc(-c2ccc3c(c2)c2ccccc2n3-c2nc(-c3ccccc3)nc(-c3ccc(-c4ccc5c(c4)oc4ccccc45)cc3)n2)cc1]

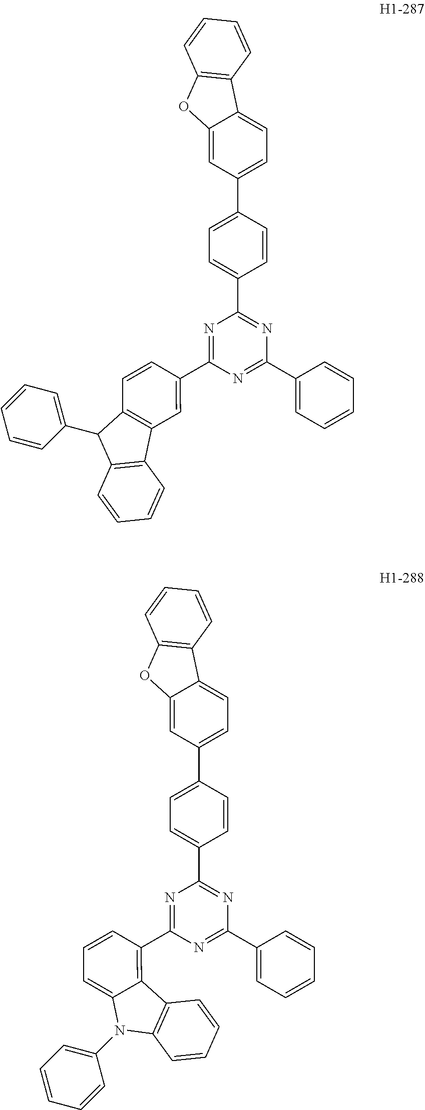 [SMILES: c1ccc(-c2nc(-c3ccc(-c4ccc5c(c4)oc4ccccc45)cc3)nc(-c3ccc4c(c3)-c3ccccc3C4c3ccccc3)n2)cc1 ; c1ccc(-c2nc(-c3ccc(-c4ccc5c(c4)oc4ccccc45)cc3)nc(-c3cccc4c3c3ccccc3n4-c3ccccc3)n2)cc1]

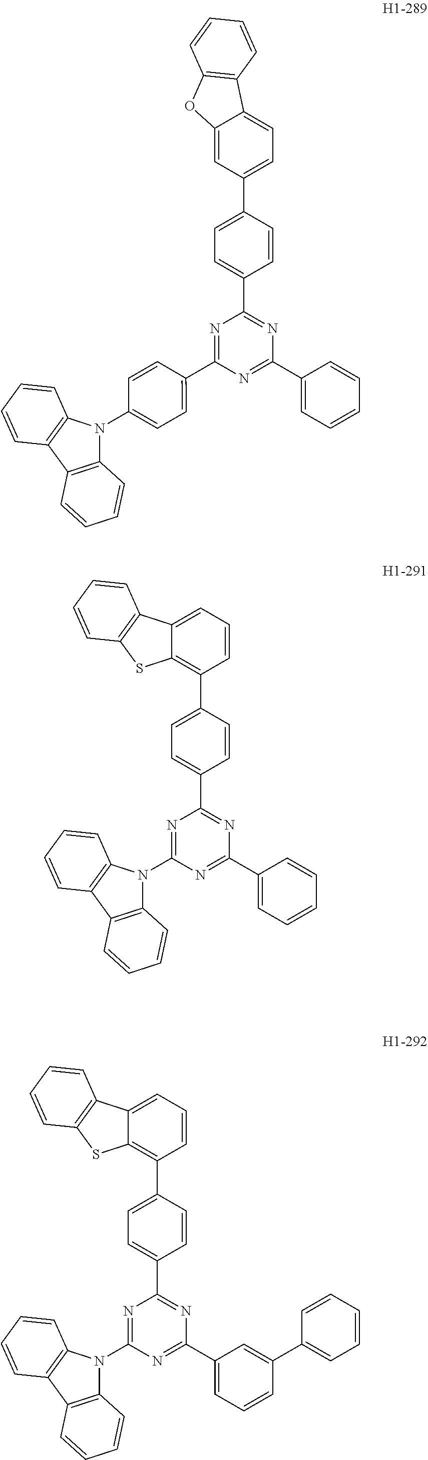 [SMILES: c1ccc(-c2nc(-c3ccc(-c4ccc5c(c4)oc4ccccc45)cc3)nc(-c3ccc(-n4c5ccccc5c5ccccc54)cc3)n2)cc1 ; c1ccc(-c2nc(-c3ccc(-c4cccc5c4sc4ccccc45)cc3)nc(-n3c4ccccc4c4ccccc43)n2)cc1 ; c1ccc(-c2cccc(-c3nc(-c4ccc(-c5cccc6c5sc5ccccc56)cc4)nc(-n4c5ccccc5c5ccccc54)n3)c2)cc1]

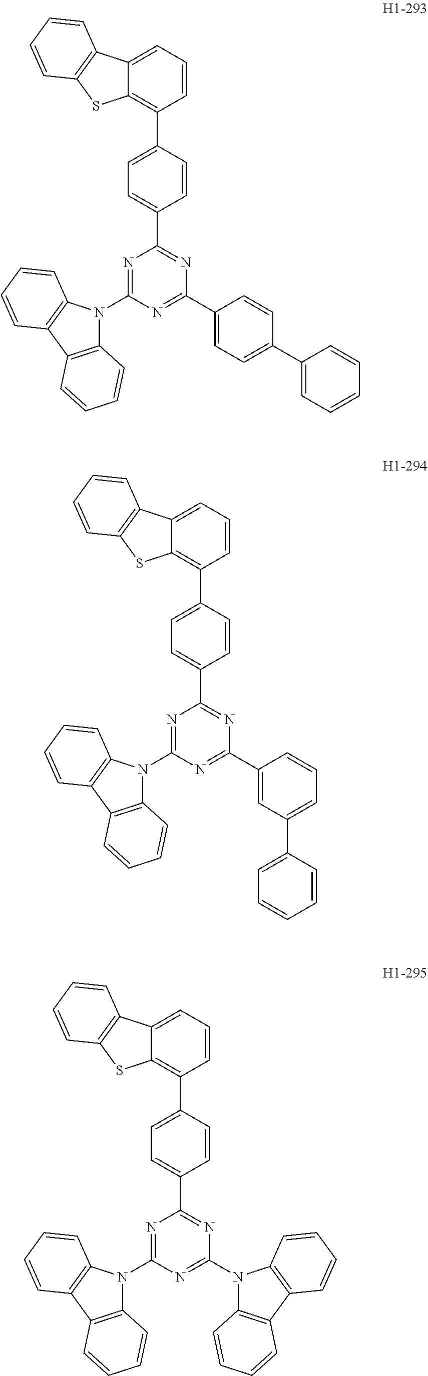 [SMILES: c1ccc(-c2ccc(-c3nc(-c4ccc(-c5cccc6c5sc5ccccc56)cc4)nc(-n4c5ccccc5c5ccccc54)n3)cc2)cc1 ; c1ccc(-c2cccc(-c3nc(-c4ccc(-c5cccc6c5sc5ccccc56)cc4)nc(-n4c5ccccc5c5ccccc54)n3)c2)cc1 ; c1ccc2c(c1)sc1c(-c3ccc(-c4nc(-n5c6ccccc6c6ccccc65)nc(-n5c6ccccc6c6ccccc65)n4)cc3)cccc12]

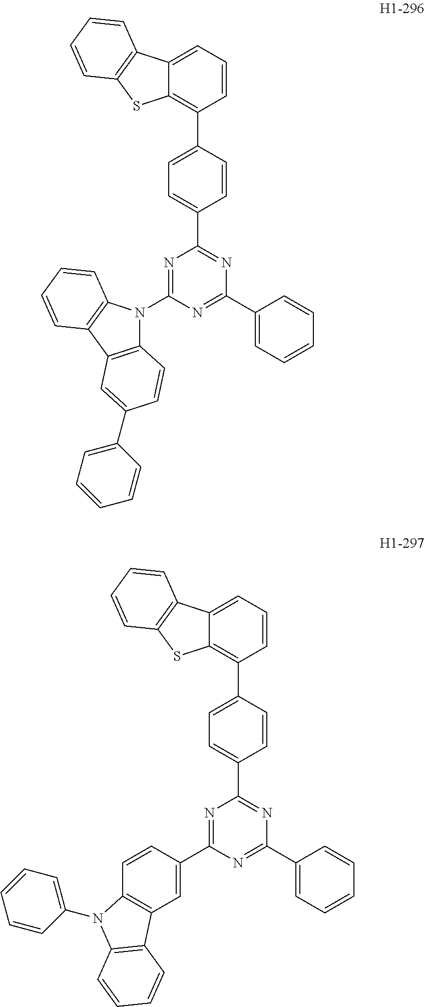 [SMILES: c1ccc(-c2ccc3c(c2)c2ccccc2n3-c2nc(-c3ccccc3)nc(-c3ccc(-c4cccc5c4sc4ccccc45)cc3)n2)cc1 ; c1ccc(-c2nc(-c3ccc(-c4cccc5c4sc4ccccc45)cc3)nc(-c3ccc4c(c3)c3ccccc3n4-c3ccccc3)n2)cc1]

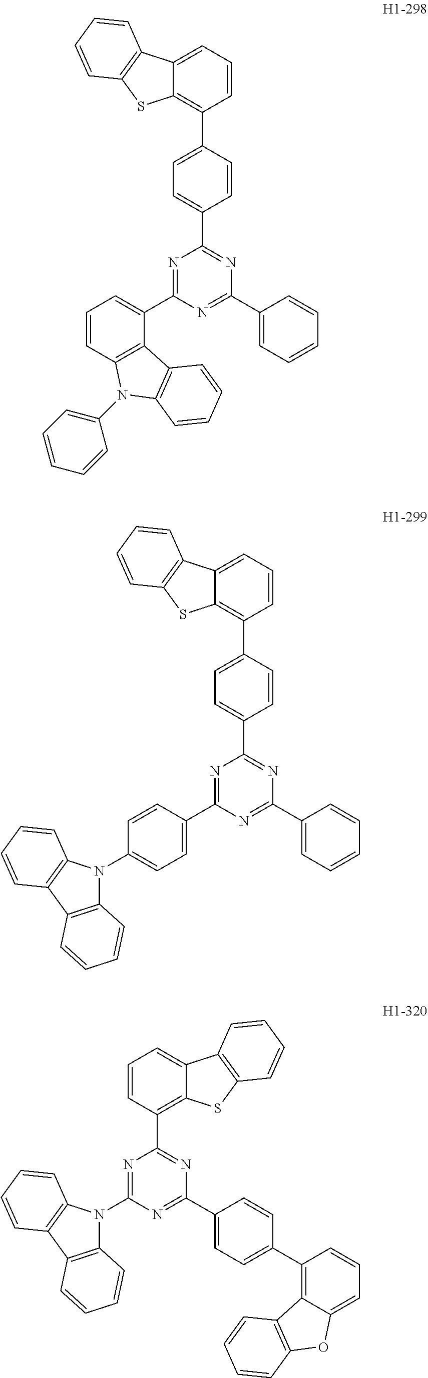 [SMILES: c1ccc(-c2nc(-c3ccc(-c4cccc5c4sc4ccccc45)cc3)nc(-c3cccc4c3c3ccccc3n4-c3ccccc3)n2)cc1 ; c1ccc(-c2nc(-c3ccc(-c4cccc5c4sc4ccccc45)cc3)nc(-c3ccc(-n4c5ccccc5c5ccccc54)cc3)n2)cc1 ; c1ccc2c(c1)oc1cccc(-c3ccc(-c4nc(-c5cccc6c5sc5ccccc56)nc(-n5c6ccccc6c6ccccc65)n4)cc3)c12]

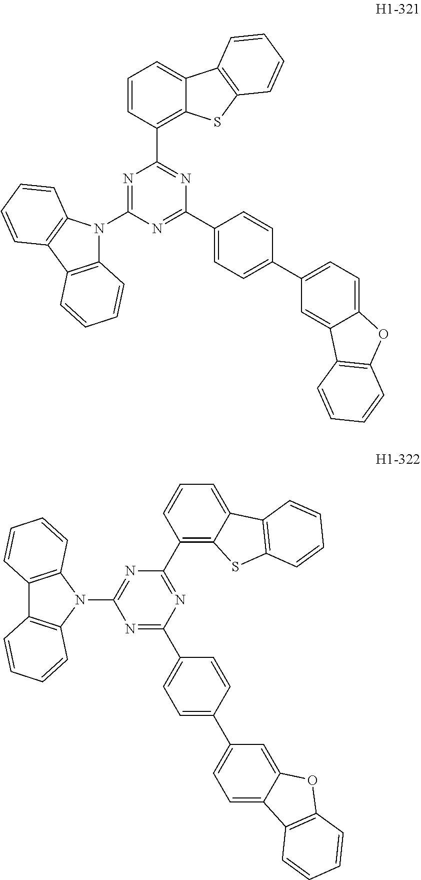 [SMILES: c1ccc2c(c1)oc1ccc(-c3ccc(-c4nc(-c5cccc6c5sc5ccccc56)nc(-n5c6ccccc6c6ccccc65)n4)cc3)cc12 ; c1ccc2c(c1)oc1cc(-c3ccc(-c4nc(-c5cccc6c5sc5ccccc56)nc(-n5c6ccccc6c6ccccc65)n4)cc3)ccc12]

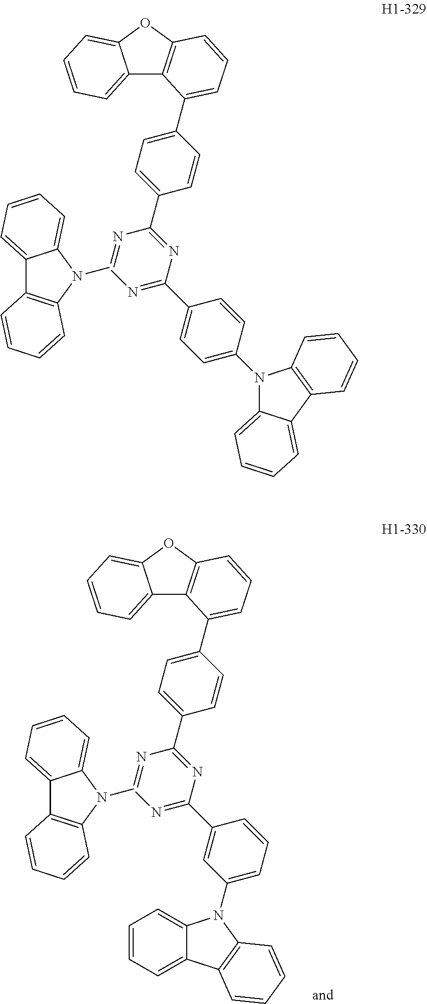

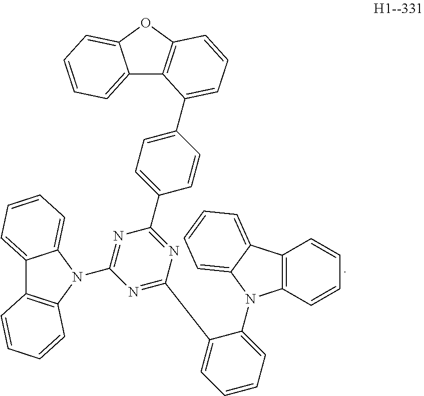 [SMILES: c1ccc(-n2c3ccccc3c3ccccc32)c(-c2nc(-c3ccc(-c4cccc5oc6ccccc6c45)cc3)nc(-n3c4ccccc4c4ccccc43)n2)c1]

14. An organic electroluminescent material comprising the organic electroluminescent compound according to claim 11.

15. An organic electroluminescent device comprising the organic electroluminescent compound according to claim 11.

16. An organic electroluminescent compound represented by the following formula 1-B: 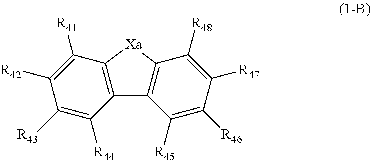
in formula 1-B,
Xₐ represent O or S;
R₄₁ to R₄₈, each independently, represent hydrogen; deuterium; a (C6-C18)aryl unsubstituted or substituted with at least one of deuterium, a (C1-C6)alkyl(s) and a (C6-C18)aryl(s); or -L_{b}-HAr_{b}, with the proviso that at least one of R₄₁ to R₄₈ represents -L_{b}-HAr_{b};
L_{b} represents a naphthylene unsubstituted or substituted with deuterium; and
HAr_{b} is represented by the following formula 1-b: 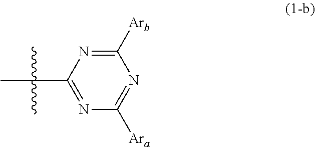
in formula 1-b,
Arₐ and Ar_{b}, each independently, represent a phenyl unsubstituted or substituted with deuterium, a naphthyl unsubstituted or substituted with deuterium, a biphenyl unsubstituted or substituted with deuterium, a terphenyl unsubstituted or substituted with deuterium, a carbazolyl unsubstituted or substituted with deuterium, a dibenzofuranyl unsubstituted or substituted with deuterium, a dibenzothiophenyl unsubstituted or substituted with deuterium, or a combination thereof, with the proviso that at least one of Arₐ and Ar_{b} comprises a carbazolyl unsubstituted or substituted with at least one of deuterium and a (C6-C18)aryl(s), a dibenzofuranyl unsubstituted or substituted with deuterium, or a dibenzothiophenyl unsubstituted or substituted with deuterium.

17. The organic electroluminescent compound according to claim 16, wherein the compound represented by formula 1-B is selected from the group consisting of the following compounds:

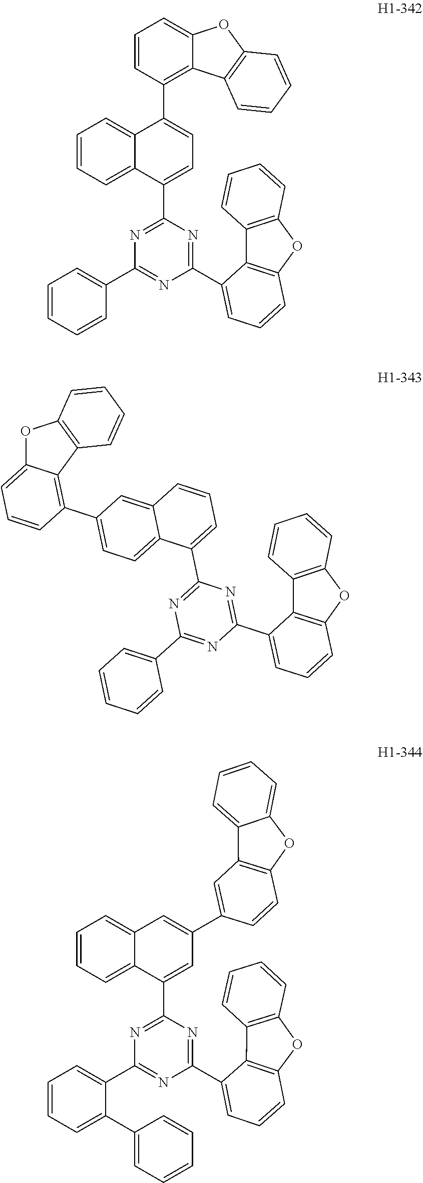 [SMILES: c1ccc(-c2nc(-c3ccc(-c4cccc5oc6ccccc6c45)c4ccccc34)nc(-c3cccc4oc5ccccc5c34)n2)cc1 ; c1ccc(-c2nc(-c3cccc4cc(-c5cccc6oc7ccccc7c56)ccc34)nc(-c3cccc4oc5ccccc5c34)n2)cc1 ; c1ccc(-c2ccccc2-c2nc(-c3cc(-c4ccc5oc6ccccc6c5c4)cc4ccccc34)nc(-c3cccc4oc5ccccc5c34)n2)cc1]

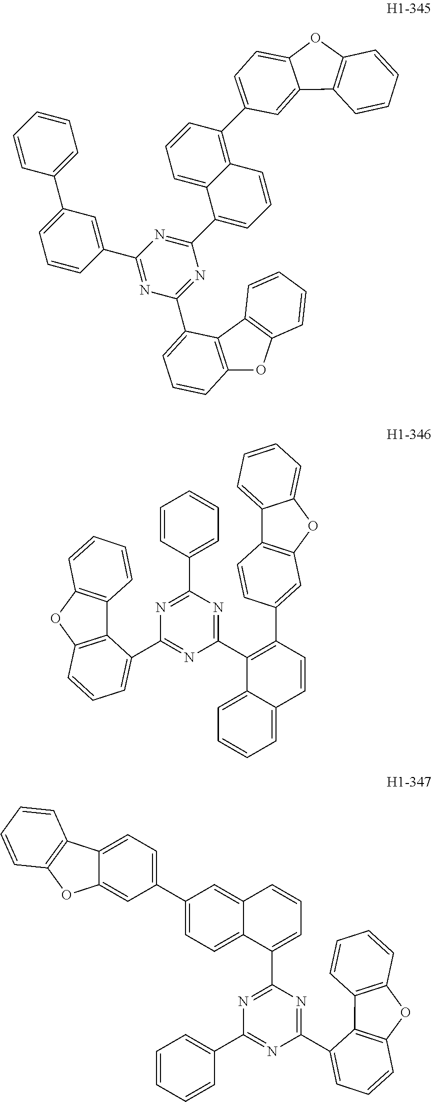 [SMILES: c1ccc(-c2cccc(-c3nc(-c4cccc5c(-c6ccc7oc8ccccc8c7c6)cccc45)nc(-c4cccc5oc6ccccc6c45)n3)c2)cc1 ; c1ccc(-c2nc(-c3c(-c4ccc5c(c4)oc4ccccc45)ccc4ccccc34)nc(-c3cccc4oc5ccccc5c34)n2)cc1 ; c1ccc(-c2nc(-c3cccc4cc(-c5ccc6c(c5)oc5ccccc56)ccc34)nc(-c3cccc4oc5ccccc5c34)n2)cc1]

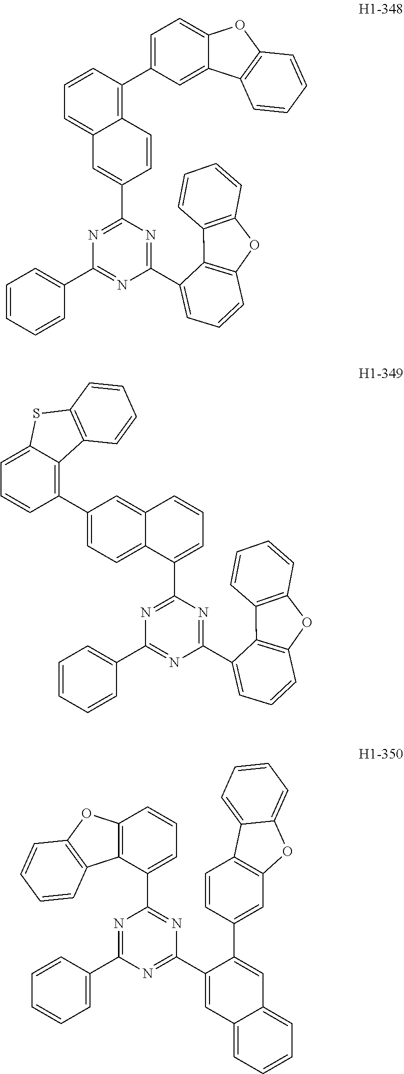 [SMILES: c1ccc(-c2nc(-c3ccc4c(-c5ccc6oc7ccccc7c6c5)cccc4c3)nc(-c3cccc4oc5ccccc5c34)n2)cc1 ; c1ccc(-c2nc(-c3cccc4cc(-c5cccc6sc7ccccc7c56)ccc34)nc(-c3cccc4oc5ccccc5c34)n2)cc1 ; c1ccc(-c2nc(-c3cc4ccccc4cc3-c3ccc4c(c3)oc3ccccc34)nc(-c3cccc4oc5ccccc5c34)n2)cc1]

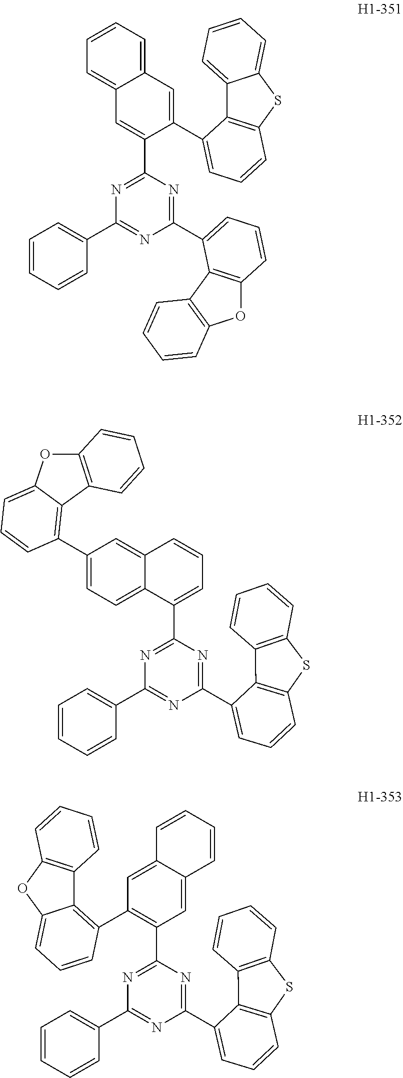 [SMILES: c1ccc(-c2nc(-c3cc4ccccc4cc3-c3cccc4sc5ccccc5c34)nc(-c3cccc4oc5ccccc5c34)n2)cc1 ; c1ccc(-c2nc(-c3cccc4cc(-c5cccc6oc7ccccc7c56)ccc34)nc(-c3cccc4sc5ccccc5c34)n2)cc1 ; c1ccc(-c2nc(-c3cc4ccccc4cc3-c3cccc4oc5ccccc5c34)nc(-c3cccc4sc5ccccc5c34)n2)cc1]

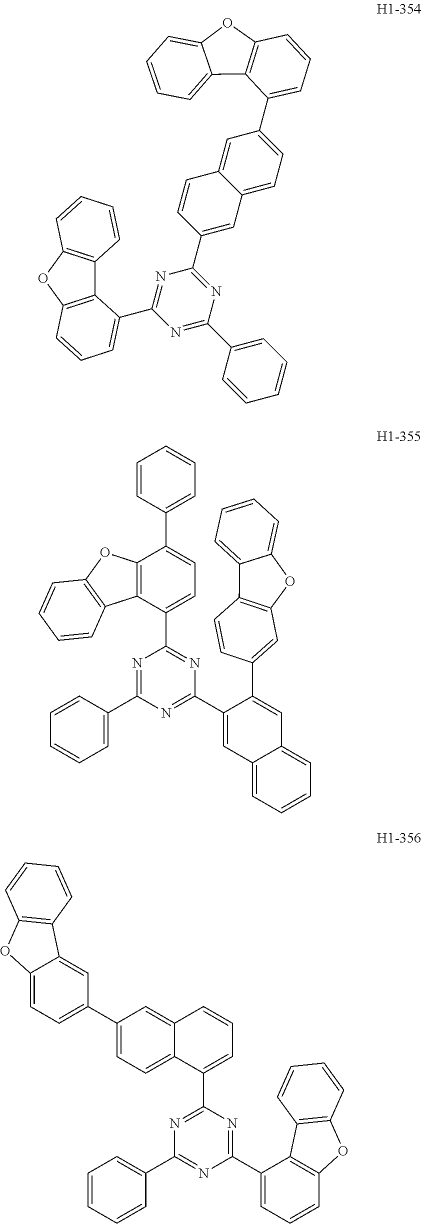 [SMILES: c1ccc(-c2nc(-c3ccc4cc(-c5cccc6oc7ccccc7c56)ccc4c3)nc(-c3cccc4oc5ccccc5c34)n2)cc1 ; c1ccc(-c2nc(-c3cc4ccccc4cc3-c3ccc4c(c3)oc3ccccc34)nc(-c3ccc(-c4ccccc4)c4oc5ccccc5c34)n2)cc1 ; c1ccc(-c2nc(-c3cccc4cc(-c5ccc6oc7ccccc7c6c5)ccc34)nc(-c3cccc4oc5ccccc5c34)n2)cc1]

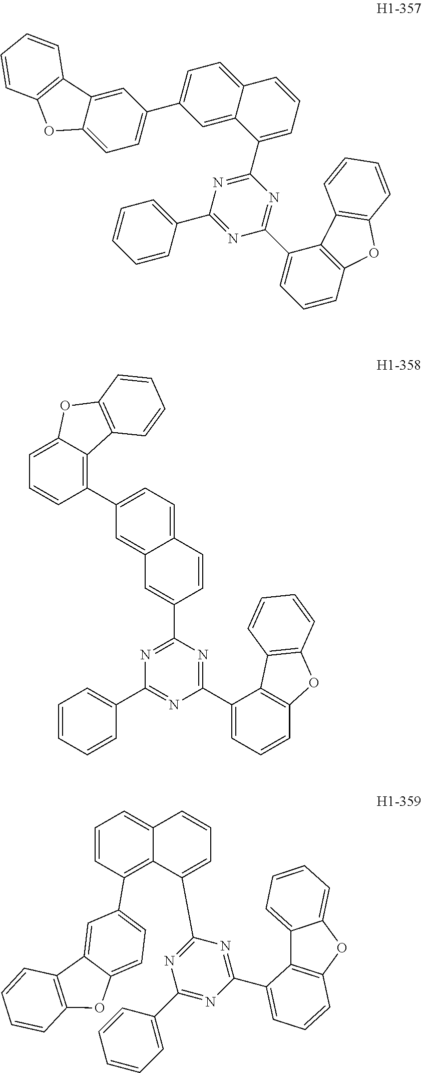 [SMILES: c1ccc(-c2nc(-c3cccc4ccc(-c5ccc6oc7ccccc7c6c5)cc34)nc(-c3cccc4oc5ccccc5c34)n2)cc1 ; c1ccc(-c2nc(-c3ccc4ccc(-c5cccc6oc7ccccc7c56)cc4c3)nc(-c3cccc4oc5ccccc5c34)n2)cc1 ; c1ccc(-c2nc(-c3cccc4cccc(-c5ccc6oc7ccccc7c6c5)c34)nc(-c3cccc4oc5ccccc5c34)n2)cc1]

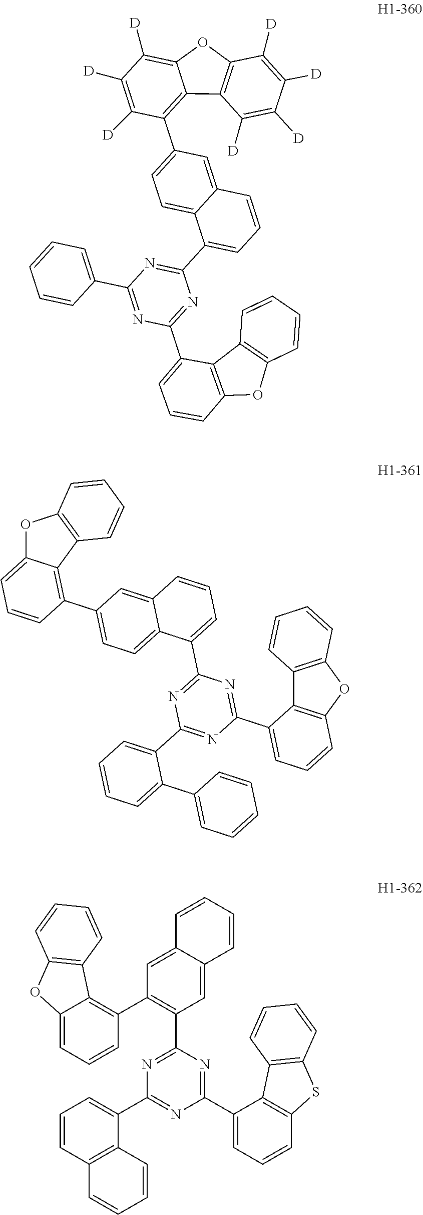 [SMILES: [2H]c1c([2H])c([2H])c2c(oc3c([2H])c([2H])c([2H])c(-c4ccc5c(-c6nc(-c7ccccc7)nc(-c7cccc8oc9ccccc9c78)n6)cccc5c4)c32)c1[2H] ; c1ccc(-c2ccccc2-c2nc(-c3cccc4cc(-c5cccc6oc7ccccc7c56)ccc34)nc(-c3cccc4oc5ccccc5c34)n2)cc1 ; c1ccc2cc(-c3cccc4oc5ccccc5c34)c(-c3nc(-c4cccc5ccccc45)nc(-c4cccc5sc6ccccc6c45)n3)cc2c1]

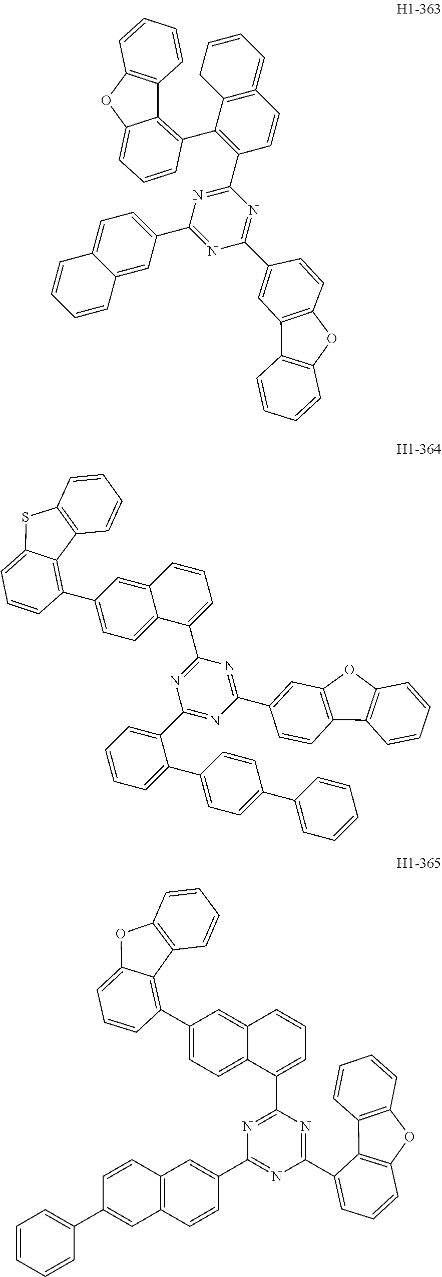

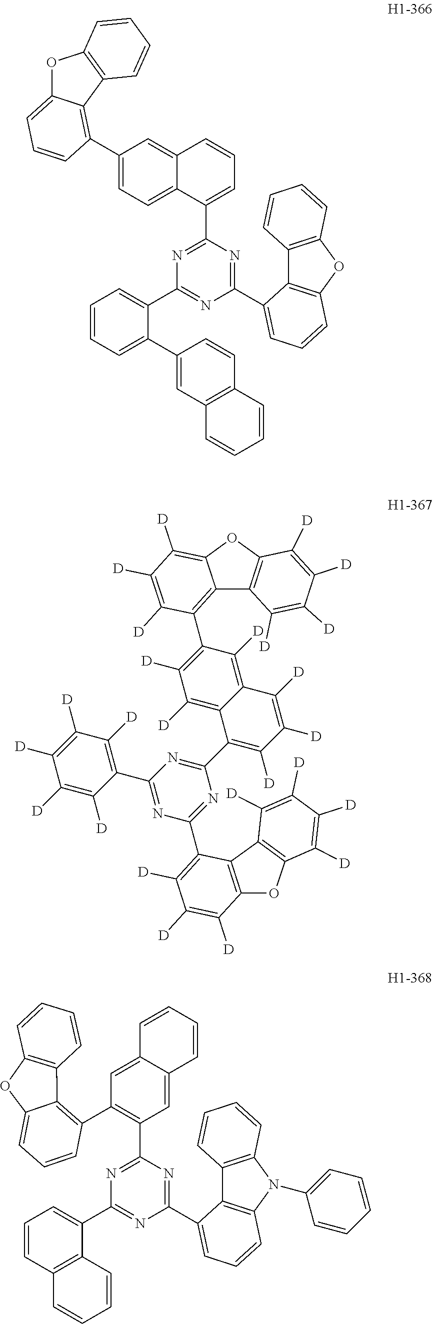 [SMILES: c1ccc(-c2nc(-c3cccc4cc(-c5cccc6oc7ccccc7c56)ccc34)nc(-c3cccc4oc5ccccc5c34)n2)c(-c2ccc3ccccc3c2)c1 ; [2H]c1c([2H])c([2H])c(-c2nc(-c3c([2H])c([2H])c([2H])c4c([2H])c(-c5c([2H])c([2H])c([2H])c6oc7c([2H])c([2H])c([2H])c([2H])c7c56)c([2H])c([2H])c34)nc(-c3c([2H])c([2H])c([2H])c4oc5c([2H])c([2H])c([2H])c([2H])c5c34)n2)c([2H])c1[2H] ; c1ccc(-n2c3ccccc3c3c(-c4nc(-c5cc6ccccc6cc5-c5cccc6oc7ccccc7c56)nc(-c5cccc6ccccc56)n4)cccc32)cc1]

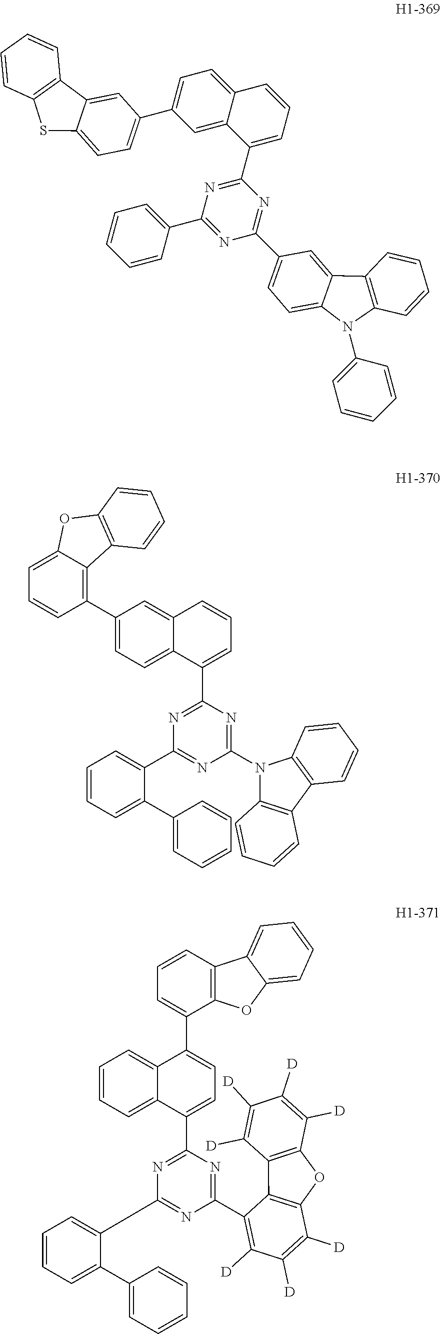 [SMILES: c1ccc(-c2nc(-c3ccc4c(c3)c3ccccc3n4-c3ccccc3)nc(-c3cccc4ccc(-c5ccc6sc7ccccc7c6c5)cc34)n2)cc1 ; c1ccc(-c2ccccc2-c2nc(-c3cccc4cc(-c5cccc6oc7ccccc7c56)ccc34)nc(-n3c4ccccc4c4ccccc43)n2)cc1 ; [2H]c1c([2H])c([2H])c2c(oc3c([2H])c([2H])c([2H])c(-c4nc(-c5ccccc5-c5ccccc5)nc(-c5ccc(-c6cccc7c6oc6ccccc67)c6ccccc56)n4)c32)c1[2H]]

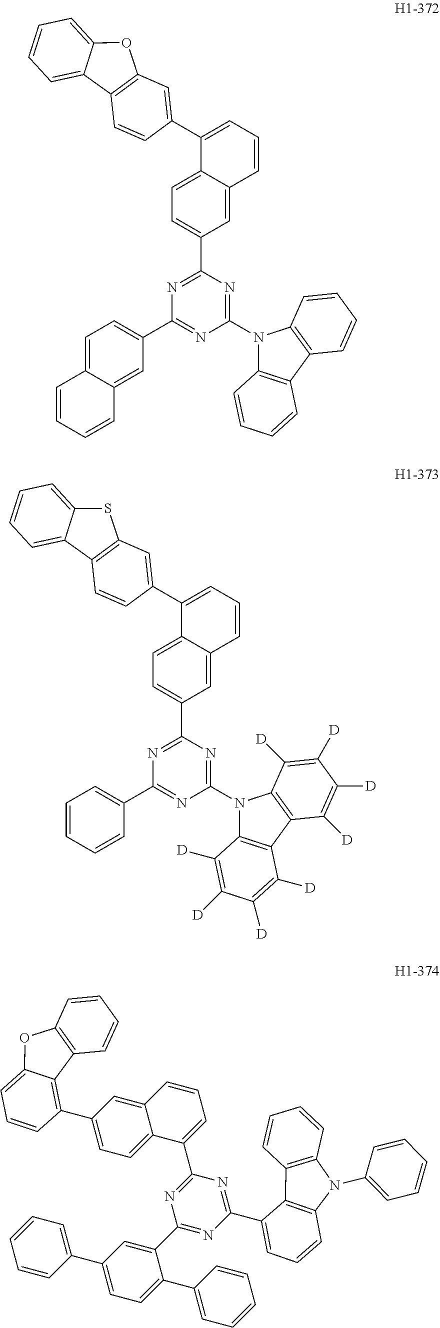 [SMILES: c1ccc2cc(-c3nc(-c4ccc5c(-c6ccc7c(c6)oc6ccccc67)cccc5c4)nc(-n4c5ccccc5c5ccccc54)n3)ccc2c1 ; [2H]c1c([2H])c([2H])c2c(c1[2H])c1c([2H])c([2H])c([2H])c([2H])c1n2-c1nc(-c2ccccc2)nc(-c2ccc3c(-c4ccc5c(c4)sc4ccccc45)cccc3c2)n1 ; c1ccc(-c2ccc(-c3ccccc3)c(-c3nc(-c4cccc5cc(-c6cccc7oc8ccccc8c67)ccc45)nc(-c4cccc5c4c4ccccc4n5-c4ccccc4)n3)c2)cc1]

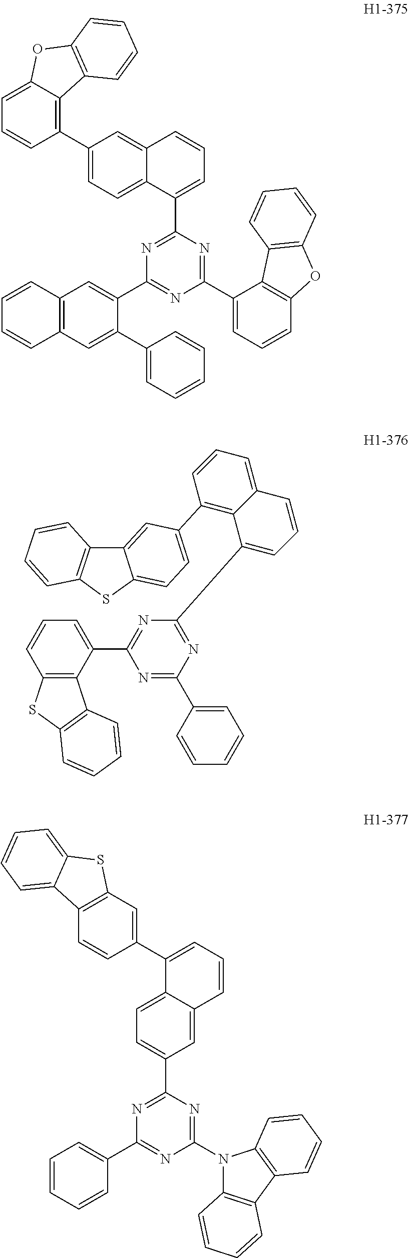 [SMILES: c1ccc(-c2cc3ccccc3cc2-c2nc(-c3cccc4cc(-c5cccc6oc7ccccc7c56)ccc34)nc(-c3cccc4oc5ccccc5c34)n2)cc1 ; c1ccc(-c2nc(-c3cccc4cccc(-c5ccc6sc7ccccc7c6c5)c34)nc(-c3cccc4sc5ccccc5c34)n2)cc1 ; c1ccc(-c2nc(-c3ccc4c(-c5ccc6c(c5)sc5ccccc56)cccc4c3)nc(-n3c4ccccc4c4ccccc43)n2)cc1]

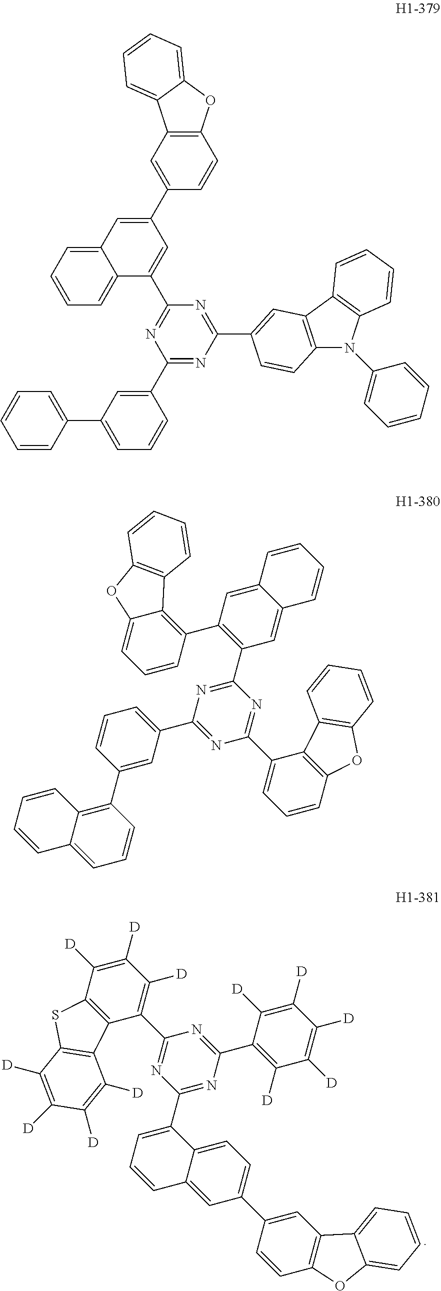 [SMILES: c1ccc(-c2cccc(-c3nc(-c4ccc5c(c4)c4ccccc4n5-c4ccccc4)nc(-c4cc(-c5ccc6oc7ccccc7c6c5)cc5ccccc45)n3)c2)cc1 ; c1cc(-c2nc(-c3cc4ccccc4cc3-c3cccc4oc5ccccc5c34)nc(-c3cccc4oc5ccccc5c34)n2)cc(-c2cccc3ccccc23)c1 ; [2H]c1c([2H])c([2H])c(-c2nc(-c3cccc4cc(-c5ccc6oc7ccccc7c6c5)ccc34)nc(-c3c([2H])c([2H])c([2H])c4sc5c([2H])c([2H])c([2H])c([2H])c5c34)n2)c([2H])c1[2H]]

18. An organic electroluminescent material comprising the organic electroluminescent compound according to claim 16.

19. An organic electroluminescent device comprising the organic electroluminescent compound according to claim 16.
